(19) United States
(12) Patent Application Publication
(10) Pub. No.: US 2014/0163012 A1
(43) Pub. Date: Jun. 12, 2014

(54) PYRIDO-/AZEPINO-BENZOFURAN AND PYRIDO-/AZEPINO-BENZOTHIOPHENE MCH-1 ANTAGONISTS, METHODS OF MAKING, AND USE THEREOF

(71) Applicant: Albany Molecular Research, Inc., Albany, NY (US)

(72) Inventors: Matthew D. SURMAN, Albany, NY (US); Emily E. FREEMAN, Guilderland, NY (US); Peter R. GUZZO, Niskayuna, NY (US); Alan J. HENDERSON, Fishers, IN (US); Mark HADDEN, Albany, NY (US)

(73) Assignee: Albany Molecular Research, Inc., Albany, NY (US)

(21) Appl. No.: 14/068,470

(22) Filed: Oct. 31, 2013

Related U.S. Application Data
(60) Provisional application No. 61/735,609, filed on Dec. 11, 2012.

(51) Int. Cl. C07D 491/048; C07D 491/147; A61K 31/444; A61K 31/513; A61K 31/501; A61K 31/496; A61K 45/06; C07D 495/04; A61K 31/55

(52) U.S. Cl. CPC C07D 491/048; C07D 495/04; C07D 491/147; A61K 31/444; A61K 31/55; A61K 31/501; A61K 31/496; A61K 45/06; A61K 31/513
USPC 514/215; 546/89; 546/80; 540/586; 544/238; 544/361; 546/62; 546/63; 514/291; 514/252.04; 514/253.03; 514/286; 514/285

(57) ABSTRACT

Novel MCH-1 receptor antagonists are disclosed. These compounds are used in the treatment of various disorders, including obesity, anxiety, depression, non-alcoholic fatty liver disease, and psychiatric disorders. Methods of making these compounds are also described.

This application claims the benefit of U.S. Provisional Patent Application Ser. No. 61/735,609, filed Dec. 11, 2012, which is hereby incorporated by reference in its entirety.

### FIELD OF THE INVENTION

This technology relates to substituted pyrido-/azepino-benzofurans and pyrido-/azepino-benzothiophenes, which are melanin-concentrating hormone (MCH-1) receptor antagonists, pharmaceutical compositions including these compounds, and methods of preparation and use thereof. The compounds are useful in the treatment of obesity, anxiety, depression, non-alcoholic fatty liver disease, and psychiatric disorders.

### BACKGROUND OF THE INVENTION

Obesity and the multitude of co-morbidities associated with obesity such as diabetes, dyslipidemia, coronary heart disease, and certain cancers are a major concern for public health. The currently available pharmaceutical therapies for the treatment of obesity have limited efficacy and side effects that limit their use. Thus, there is a significant medical need for better pharmacotherapy for obesity.

Obesity has associated with it, economic and social costs. Obese people, an increasing proportion of most western societies, are regarded as having out of control feeding habits often associated with low self-esteem. Moreover, obese persons are more likely to have medical problems associated with or exacerbated by the excess body weight. Examples of medical conditions caused, exacerbated, or triggered by excessive weight include bone fractures, pains in the knee joints, arthritis, increased risk of hypertension, artherosclerosis, stroke, and diabetes.

Melanin-concentrating hormone (MCH) has been identified as an orexigenic peptide that exerts an effect on food intake and body weight regulation. MCH is a cyclic 19 amino acid neuropeptide expressed in the zona incerta and lateral hypothalamus in response to both energy restriction and leptin deficiency. MCH is known to stimulate feeding when injected into the lateral ventricle of rats and the mRNA for MCH is upregulated in the hypothalamus of genetically obese mice (ob/ob) and in fasted control and ob/ob animals. In addition, animals treated with MCH show increases in glucose, insulin and leptin levels, mimicking human metabolic syndrome (Gomori, “Chronic Infusion of MCH Causes Obesity in Mice,” Am. J. Physiol. Endocrinol. Metab., 284:E583 (2002)). Mice lacking MCH are hypophagic and lean with increased metabolic rate, whereas animals over-expressing MCH gain excess weight on both standard and high fat diets. MCH is thought to have effects on other nervous system functions as well (Rocksz, “Biological Examination of Melanin Concentrating Hormone 1: Multi-tasking from the Hypothalamus,” Drug News Perspect., 19(5):273 (2006)). An orphan G-protein coupled receptor (GPCR) was recently identified as a receptor for MCH. Disruption of the binding between MCH and the MCH receptor, i.e. MCH antagonism, may thus be used to counteract the effects of MCH (McBriar, “Recent Advances in the Discovery of Melanin-Concentrating Hormone Receptor Antagonists,” Curr. Opin. Drug Disc. & Dev., 9(4):496 (2006)).

The current preferred treatment for obesity as well as Type II non-insulin dependent diabetes is diet and exercise with a view toward weight reduction and improved insulin sensitivity for diabetics. Patient compliance, however, is usually poor. The problem is compounded by the fact that there is currently only a single medication approved for the long term treatment of obesity (orlistat (XENICAL™)).

This technology is directed to overcoming these and other deficiencies in the art.

### SUMMARY OF THE INVENTION

This technology relates to a compound of formula (I):

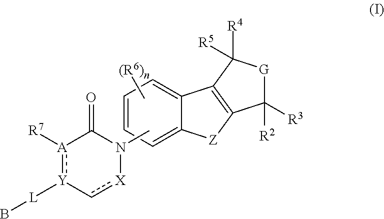

wherein
R²-R⁵ and R⁹-R¹² are each, independently, selected from the group consisting of H, halogen, —OR¹³, —NR¹³R¹⁴, —NR¹³C(O)R¹⁴, —NR¹³C(O)₂R¹⁴, —NR¹⁵C(O)NR¹⁴R¹⁵, —S(O)_{q}R¹⁴, —CN, —C(O)R¹⁴, —C(O)NR¹³R¹⁴, C₁-C₆ alkyl, C₂-C₆ alkenyl, C₂-C₆ alkynyl, C₃-C₆ cycloalkyl, C₄-C₇ cycloalkylalkyl, heterocyclyl, aryl, and heteroaryl, wherein each of C₁-C₆ alkyl, C₂-C₆ alkenyl, C₂-C₆ alkynyl, C₃-C₆ cycloalkyl, C₄-C₇ cycloalkylalkyl, heterocyclyl, aryl, and heteroaryl is optionally substituted with from 1 to 3 substituents independently selected at each occurrence thereof from C₁-C₃ alkyl, halogen, —CN, —OR¹⁶, —NR¹⁶R¹⁷, and phenyl which is optionally substituted 1-3 times with halogen, C₁-C₄ alkyl, C₁-C₄ haloalkyl, C₁-C₄ alkoxy, —CN, —OR¹⁶, or —NR¹⁶R¹⁷; or R² and R³ or R⁴ and R⁵ can combine to form an oxo, thio, imine, cycloalkyl, or heterocycle group containing from 1 to 5 heteroatoms selected from the group consisting of oxygen, nitrogen, and sulfur; or any one of R², R³, R⁴, or R⁵ can combine with any one of R⁹, R¹⁰, R¹¹, or R¹² to form —(CH₂)ᵣ—;
R⁶ is independently selected at each location from the group consisting of H, halogen, —OR¹³, —NR¹³C(O)R¹⁴, —NR¹³C(O)₂R¹⁴, —NR¹⁵C(O)NR¹⁴R¹⁵, —S(O)_{q}R¹⁴, —CN, —C(O)R¹⁴, —C(O)NR¹³R¹⁴, C₁-C₆ alkyl, C₂-C₆ alkenyl, C₂-C₆ alkynyl, C₃-C₆ cycloalkyl, C₄-C₇ cycloalkylalkyl, heterocyclyl, aryl, and heteroaryl, wherein each of C₁-C₆ alkyl, C₂-C₆ alkenyl, C₂-C₆ alkynyl, C₃-C₆ cycloalkyl, C₄-C₇ cycloalkylalkyl, heterocyclyl, aryl, and heteroaryl is optionally substituted with from 1 to 3 substituents independently selected at each occurrence thereof from C₁-C₃ alkyl, halogen, —CN, —OR⁶, —NR¹⁶R¹⁷, and phenyl which is optionally substituted 1-3 times with halogen, C₁-C₄ alkyl, C₁-C₄ haloalkyl, C₁-C₄ alkoxy, —CN, —OR¹⁶, or NR¹⁶R¹⁷;
R⁷ is optionally present and, if present, is selected from the group consisting of H, halogen, —OR¹³, —NR¹³R¹⁴, —NR¹³C(O)R¹⁴, —NR¹³C(O)₂R¹⁴, —NR¹⁵C(O)NR¹⁴R¹⁵, —S(O)_{q}R¹⁴, —CN, —C(O)R¹⁴, —C(O)NR¹³R¹⁴, C₁-C₆ alkyl, C₂-C₆ alkenyl, C₂-C₆ alkynyl, C₃-C₆ cycloalkyl, C₄-C₇ cycloalkylalkyl, heterocyclyl, aryl, and heteroaryl, wherein each of C₁-C₆ alkyl, C₂-C₆ alkenyl, C₂-C₆ alkynyl, C₃-C₆ cycloalkyl, C₄-C₇ cycloalkylalkyl, heterocyclyl, aryl, and heteroaryl is optionally substituted with from 1 to 3 substituents independently selected at each occurrence thereof from C₁-C₃ alkyl, halogen, —CN, —OR¹⁶, —NR¹⁶R¹⁷, and phenyl which is optionally substituted 1-3 times with halogen, C₁-C₄ alkyl, C₁-C₄ haloalkyl, C₁-C₄ alkoxy, —CN, —OR¹⁶, or —NR¹⁶R¹⁷;
R⁸ is selected from the group consisting of H, —S(O)_{q}R¹⁴, —C(O)R¹⁴, —C(O)NR¹³R¹⁴, C₁-C₆ alkyl, C₂-C₆ alkenyl, C₂-C₆ alkynyl, C₃-C₆ cycloalkyl, C₄-C₇ cycloalkylalkyl, heterocyclyl, and heteroaryl, wherein each of C₁-C₆ alkyl, C₂-C₆ alkenyl, C₂-C₆ alkynyl, C₃-C₆ cycloalkyl, C₄-C₇ cycloalkylalkyl, heterocyclyl, and heteroaryl is optionally substituted with from 1 to 3 substituents independently selected at each occurrence thereof from C₁-C₃ alkyl, halogen, —CN, —OR¹⁶, —NR¹⁶R¹⁷, and phenyl which is optionally substituted 1-3 times with halogen, C₁-C₄ alkyl, C₁-C₄ haloalkyl, C₁-C₄ alkoxy, —CN, —OR¹⁶, or NR¹⁶R¹⁷; or
R⁸ and one of R², R³, R⁴, and R⁵ can combine to form a 3- to 7-membered heterocycle, wherein the 3- to 7-membered heterocycle includes from 1 to 2 heteroatoms selected from the group consisting of N, O, and S and is optionally substituted with from 1 to 10 substituents independently selected at each occurrence thereof from H, halogen, —OR¹³, —NR¹³R¹⁴, —NR¹³C(O)R¹⁴, —NR¹³C(O)₂R¹⁴, —NR¹⁵C(O)NR¹⁴R¹⁵, —S(O)_{q}R¹⁴, —CN, —C(O)R¹⁴, —C(O)NR¹³R¹⁴, C₁-C₆ alkyl, C₂-C₆ alkenyl, C₂-C₆ alkynyl, C₃-C₆ cycloalkyl, C₄-C₇ cycloalkylalkyl, heterocyclyl, aryl, and heteroaryl, wherein each of C₁-C₆ alkyl, C₂-C₆ alkenyl, C₂-C₆ alkynyl, C₃-C₆ cycloalkyl, C₄-C₇ cycloalkylalkyl, heterocyclyl, aryl, and heteroaryl is optionally substituted with from 1 to 3 substituents independently selected at each occurrence thereof from C₁-C₃ alkyl, halogen, —CN, —OR¹⁶, —NR¹⁶R¹⁷, and phenyl which is optionally substituted 1-3 times with halogen, C₁-C₄ alkyl, C₁-C₄ haloalkyl, C₁-C₄ alkoxy, —CN, —OR¹⁶, or —NR¹⁶R¹⁷;
R¹³ is H, C₁-C₄ alkyl, C₁-C₄ haloalkyl, C₁-C₄ alkoxyalkyl, C₃-C₆ cycloalkyl, C₄-C₇ cycloalkylalkyl, —C(O)R¹⁵, phenyl, or benzyl, wherein phenyl or benzyl is optionally substituted 1 to 3 times with halogen, cyano, C₁-C₄ alkyl, C₁-C₄ haloalkyl, or C₁-C₄ alkoxy;
R¹⁴ is H, C₁-C₄ alkyl, C₁-C₄ haloalkyl, C₁-C₄ alkoxyalkyl, C₃-C₆ cycloalkyl, C₄-C₇ cycloalkylalkyl, phenyl, or benzyl, wherein phenyl or benzyl is optionally substituted 1 to 3 times with halogen, cyano, C₁-C₄ alkyl, C₁-C₄ haloalkyl, or C₁-C₄ alkoxy;
R¹⁵ is C₁-C₄ alkyl, C₁-C₄ haloalkyl, or phenyl;
R¹⁶ and R¹⁷ are each independently H, C₁-C₄ alkyl, C₁-C₄ haloalkyl, C₁-C₄ alkoxyalkyl, C₃-C₆ cycloalkyl, C₄-C₇ cycloalkylalkyl, —C(O)R¹⁵, phenyl, or benzyl, wherein phenyl or benzyl is optionally substituted from 1 to 3 times with a substituent selected independently at each occurrence thereof from the group consisting of halogen, cyano, C₁-C₄ alkyl, C₁-C₄ haloalkyl, and C₁-C₄ alkoxy;
R¹⁸ is selected from the group consisting of H, halogen, —OR¹³, —NR¹³R¹⁴—NR¹³C(O)R¹⁴, —NR¹³C(O)₂R¹⁴, —NR¹⁴C(O)NR¹⁴R¹⁵, —S(O)_{q}R¹⁴, —CN, —C(O)R¹⁴, —C(O)NR¹³R¹⁴, C₁-C₆ alkyl, C₂-C₆ alkenyl, C₂-C₆ alkynyl, C₃-C₆ cycloalkyl, C₄-C₇ cycloalkylalkyl, heterocyclyl, aryl, and heteroaryl, wherein each of C₁-C₆ alkyl, C₂-C₆ alkenyl, C₂-C₆ alkynyl, C₃-C₆ cycloalkyl, C₄-C₇ cycloalkylalkyl, heterocyclyl, aryl, and heteroaryl is optionally substituted with from 1 to 3 substituents independently selected at each occurrence thereof from C₁-C₃ alkyl, halogen, —CN, —OR¹⁶, —NR¹⁶R¹⁷, and phenyl which is optionally substituted 1-3 times with halogen, C₁-C₄ alkyl, C₁-C₄ haloalkyl, C₁-C₄ alkoxy, —CN, —OR⁸, or —NR⁸R⁹;
G is —NR⁸—CR⁹R¹⁰—, —CR⁹R¹⁰—NR⁸—, —NR⁸—CR⁹R¹⁰—CR¹¹R¹²—, —CR⁹R¹⁰—NR⁸—CR¹¹R¹²—, or —CR⁹R¹⁰—CR¹¹R¹²—NR⁸—;

### X is CR¹⁸, C(R¹⁸)₂, N, or NR¹⁸;

### Y is CR¹⁸, C, or N;

### Z is O or S;

L is —(CH₂)ₚ—O—, —(CH₂)ₚ—, —CH═CH—, or a bond;

### A is C, CH, or N;

B is aryl, heteroaryl, heterocyclyl, or cycloalkyl, wherein each of the aryl, heteroaryl, heterocyclyl, or cycloalkyl is optionally substituted with from 1 to 3 substituents selected from the group consisting of H, alkoxy, —S-alkyl, optionally substituted C₁-C₆ alkyl, halogen, —CF₃, —OCF₃, and —CN;
n is 0, 1, 2, or 3;
p is from 1 to 4;
q is 0, 1, or 2;
r is from 1 to 4; and [symbol]
represents an optional double bond,
or an oxide thereof, a pharmaceutically acceptable salt thereof, a solvate thereof, or prodrug thereof.

Additional aspects include pharmaceutical compositions comprising a compound as described above and a pharmaceutically acceptable carrier and, optionally, one or more additional additive agent(s) as discussed below.

This technology also relates to a method of treating a disease or condition which is susceptible to treatment with a MCH-1 receptor antagonist. This method involves selecting a patient with a disease or condition which is susceptible to treatment with a MCH-1 antagonist and administering to the patient a therapeutically effective amount of a compound of formula I or a pharmaceutically acceptable salt thereof.

This technology also relates to a method of treating obesity in a subject in need of weight loss. This method involves selecting a patient in need of weight loss and administering to the patient a therapeutically effective amount of a compound of formula I or a pharmaceutically acceptable salt thereof.

Yet another aspect of this technology relates to a method of treating obesity in a subject who has experienced weight loss. This method involves selecting a patient who has experienced weight loss and administering to the patient a therapeutically effective amount of a compound of formula I or a pharmaceutically acceptable salt thereof.

A further aspect of this technology relates to a method of treating anxiety. This method involves selecting a patient with anxiety and administering to the patient a therapeutically effective amount of a compound of formula I or a pharmaceutically acceptable salt thereof.

This technology also relates to a method of treating depression. This method involves selecting a patient with depression and administering to the patient a therapeutically effective amount of a compound of formula I or a pharmaceutically acceptable salt thereof.

Another aspect of this technology relates to a method of treating non-alcoholic fatty liver disease. This method involves selecting a patient who has non-alcoholic fatty liver disease and administering to the patient a therapeutically effective amount of a compound of formula I or a pharmaceutically acceptable salt thereof.

A further aspect of this technology relates to a process for preparation of a product compound of formula I which includes treating a first intermediate compound of formula II:

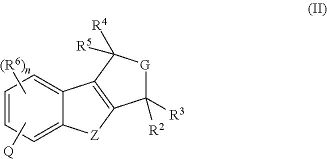

wherein Q is a halogen, under conditions effective to form the product compound.

It has now been found that compounds of formula I are MCH-1 receptor antagonists. This technology provides compounds that bind to the MCH-1 receptor with high affinity. The compounds provided by formula I are useful for the treatment of obesity, anxiety, depression, psychiatric disorders, and other disorders described herein. In particular, it is contemplated that the compounds described herein will be effective in treating obesity, including weight loss and maintenance of weight loss in patients who have been diagnosed with obesity by the one or more of the following measurements: an increased body mass index, increased waist circumference (an indicator of intra-abdominal fat), Dual Energy X-Ray Absorptiometry (DEXA), and trucal (android) fat mass. It is further contemplated that the compounds described herein will be effective in inducing improvements in certain factors measured in these tests.

### DETAILED DESCRIPTION OF THE INVENTION

This technology relates to a compound of formula (I)

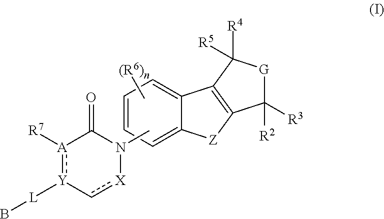

wherein
R²-R⁵ and R⁹-R¹² are each, independently, selected from the group consisting of H, halogen, —OR¹³, —NR¹³R¹⁴, —NR¹³C(O)R¹⁴, —NR¹³C(O)₂R¹⁴, —NR¹⁵C(O)NR¹⁴R¹⁵, —S(O)_{q}R¹⁴, —CN, —C(O)R¹⁴, —C(O)NR¹³R¹⁴, C₁-C₆ alkyl, C₂-C₆ alkenyl, C₂-C₆ alkynyl, C₃-C₆ cycloalkyl, C₄-C₇ cycloalkylalkyl, heterocyclyl, aryl, and heteroaryl, wherein each of C₁-C₆ alkyl, C₂-C₆ alkenyl, C₂-C₆ alkynyl, C₃-C₆ cycloalkyl, C₄-C₇ cycloalkylalkyl, heterocyclyl, aryl, and heteroaryl is optionally substituted with from 1 to 3 substituents independently selected at each occurrence thereof from C₁-C₃ alkyl, halogen, —CN, —OR¹⁶, —NR¹⁶R¹⁷, and phenyl which is optionally substituted 1-3 times with halogen, C₁-C₄ alkyl, C₁-C₄ haloalkyl, C₁-C₄ alkoxy, —CN, —OR¹⁶, or —NR¹⁶R¹⁷; or R² and R³ or R⁴ and R⁵ can combine to form an oxo, thio, imine, cycloalkyl, or heterocycle group containing from 1 to 5 heteroatoms selected from the group consisting of oxygen, nitrogen, and sulfur; or any one of R², R³, R⁴, or R⁵ can combine with any one of R⁹, R¹⁰, R¹¹, or R¹² to form —(CH₂)ᵣ—;
R⁶ is independently selected at each location from the group consisting of H, halogen, —OR¹³, —NR¹³C(O)R¹⁴, —NR¹³C(O)₂R¹⁴, —NR¹⁵C(O)NR¹⁴R¹⁵, —S(O)_{q}R¹⁴, —CN, —C(O)R¹⁴, —C(O)NR¹³R¹⁴, C₁-C₆ alkyl, C₂-C₆ alkenyl, C₂-C₆ alkynyl, C₃-C₆ cycloalkyl, C₄-C₇ cycloalkylalkyl, heterocyclyl, aryl, and heteroaryl, wherein each of C₁-C₆ alkyl, C₂-C₆ alkenyl, C₂-C₆ alkynyl, C₃-C₆ cycloalkyl, C₄-C₇ cycloalkylalkyl, heterocyclyl, aryl, and heteroaryl is optionally substituted with from 1 to 3 substituents independently selected at each occurrence thereof from C₁-C₃ alkyl, halogen, —CN, —OR¹⁶, —NR¹⁶R¹⁷, and phenyl which is optionally substituted 1-3 times with halogen, C₁-C₄ alkyl, C₁-C₄ haloalkyl, C₁-C₄ alkoxy, —CN, —OR¹⁶, or —NR¹⁶R¹⁷;
R⁷ is optionally present and, if present, is selected from the group consisting of H, halogen, —OR¹³, —NR¹³R¹⁴, —NR¹³C(O)R¹⁴, —NR¹³C(O)₂R¹⁴, —NR¹⁵C(O)NR¹⁴R¹⁵, —S(O)_{q}R¹⁴, —CN, —C(O)R¹⁴, —C(O)NR¹³R¹⁴, C₁-C₆ alkyl, C₂-C₆ alkenyl, C₂-C₆ alkynyl, C₃-C₆ cycloalkyl, C₄-C₇ cycloalkylalkyl, heterocyclyl, aryl, and heteroaryl, wherein each of C₁-C₆ alkyl, C₂-C₆ alkenyl, C₂-C₆ alkynyl, C₃-C₆ cycloalkyl, C₄-C₇ cycloalkylalkyl, heterocyclyl, aryl, and heteroaryl is optionally substituted with from 1 to 3 substituents independently selected at each occurrence thereof from C₁-C₃ alkyl, halogen, —CN, —OR¹⁶, —NR¹⁶R¹⁷, and phenyl which is optionally substituted 1-3 times with halogen, C₁-C₄ alkyl, C₁-C₄ haloalkyl, C₁-C₄ alkoxy, —CN, —OR¹⁶, or —NR¹⁶R¹⁷;
R⁸ is selected from the group consisting of H, —S(O)_{q}R¹⁴, —C(O)R¹⁴, —C(O)NR¹³R¹⁴C₁-C₆ alkyl, C₂-C₆ alkenyl, C₂-C₆ alkynyl, C₃-C₆ cycloalkyl, C₄-C₇ cycloalkylalkyl, heterocyclyl, and heteroaryl, wherein each of C₁-C₆ alkyl, C₂-C₆ alkenyl, C₂-C₆ alkynyl, C₃-C₆ cycloalkyl, C₄-C₇ cycloalkylalkyl, heterocyclyl, and heteroaryl is optionally substituted with from 1 to 3 substituents independently selected at each occurrence thereof from C₁-C₃ alkyl, halogen, —CN, —OR¹⁶, —NR¹⁶R¹⁷, and phenyl which is optionally substituted 1-3 times with halogen, C₁-C₄ alkyl, C₁-C₄ haloalkyl, C₁-C₄ alkoxy, —CN, —OR¹⁶, or NR¹⁶R¹⁷; or
R⁸ and one of R², R³, R⁴, and R⁵ can combine to form a 3- to 7-membered heterocycle, wherein the 3- to 7-membered heterocycle includes from 1 to 2 heteroatoms selected from the group consisting of N, O, and S and is optionally substituted with from 1 to 10 substituents independently selected at each occurrence thereof from H, halogen, —OR¹³, —NR¹³R¹⁴, —NR¹³C(O)R¹⁴, —NR¹³C(O)₂R¹⁴, —NR¹⁵C(O)NR¹⁴R¹⁵, —S(O)_{q}R¹⁴, —CN, —C(O)R¹⁴, —C(O)NR¹³R¹⁴, C₁-C₆ alkyl, C₂-C₆ alkenyl, C₂-C₆ alkynyl, C₃-C₆ cycloalkyl, C₄-C₇ cycloalkylalkyl, heterocyclyl, aryl, and heteroaryl, wherein each of C₁-C₆ alkyl, C₂-C₆ alkenyl, C₂-C₆ alkynyl, C₃-C₆ cycloalkyl, C₄-C₇ cycloalkylalkyl, heterocyclyl, aryl, and heteroaryl is optionally substituted with from 1 to 3 substituents independently selected at each occurrence thereof from C₁-C₃ alkyl, halogen, —CN, —OR¹⁶, —NR¹⁶R¹⁷, and phenyl which is optionally substituted 1-3 times with halogen, C₁-C₄ alkyl, C₁-C₄ haloalkyl, C₁-C₄ alkoxy, —CN, —OR¹⁶, or —NR¹⁶R¹⁷;
R¹³ is H, C₁-C₄ alkyl, C₁-C₄ haloalkyl, C₁-C₄ alkoxyalkyl, C₃-C₆ cycloalkyl, C₄-C₇ cycloalkylalkyl, —C(O)R¹⁵, phenyl, or benzyl, wherein phenyl or benzyl is optionally substituted 1 to 3 times with halogen, cyano, C₁-C₄ alkyl, C₁-C₄ haloalkyl, or C₁-C₄ alkoxy;
R¹⁴ is H, C₁-C₄ alkyl, C₁-C₄ haloalkyl, C₁-C₄ alkoxyalkyl, C₃-C₆ cycloalkyl, C₄-C₇ cycloalkylalkyl, phenyl, or benzyl, wherein phenyl or benzyl is optionally substituted 1 to 3 times with halogen, cyano, C₁-C₄ alkyl, C₁-C₄ haloalkyl, or C₁-C₄ alkoxy;
R¹⁵ is C₁-C₄ alkyl, C₁-C₄ haloalkyl, or phenyl;
R¹⁶ and R¹⁷ are each independently H, C₁-C₄ alkyl, C₁-C₄ haloalkyl, C₁-C₄ alkoxyalkyl, C₃-C₆ cycloalkyl, C₄-C₇ cycloalkylalkyl, —C(O)R¹⁵, phenyl, or benzyl, wherein phenyl or benzyl is optionally substituted from 1 to 3 times with a substituent selected independently at each occurrence thereof from the group consisting of halogen, cyano, C₁-C₄ alkyl, C₁-C₄ haloalkyl, and C₁-C₄ alkoxy;
R¹⁸ is selected from the group consisting of H, halogen, —OR¹³, —NR¹³R¹⁴—NR¹³C(O)R¹⁴, —NR¹³C(O)₂R¹⁴, —NR¹⁴C(O)NR¹⁴R¹⁵, —S(O)_{q}R¹⁴, —CN, —C(O)R¹⁴, —C(O)NR¹³R¹⁴, C₁-C₆ alkyl, C₂-C₆ alkenyl, C₂-C₆ alkynyl, C₃-C₆ cycloalkyl, C₄-C₇ cycloalkylalkyl, heterocyclyl, aryl, and heteroaryl, wherein each of C₁-C₆ alkyl, C₂-C₆ alkenyl, C₂-C₆ alkynyl, C₃-C₆ cycloalkyl, C₄-C₇ cycloalkylalkyl, heterocyclyl, aryl, and heteroaryl is optionally substituted with from 1 to 3 substituents independently selected at each occurrence thereof from C₁-C₃ alkyl, halogen, —CN, —OR¹⁶, —NR¹⁶R¹⁷, and phenyl which is optionally substituted 1-3 times with halogen, C₁-C₄ alkyl, C₁-C₄ haloalkyl, C₁-C₄ alkoxy, —CN, —OR⁸, or —NR⁸R⁹;
G is —NR⁸—CR⁹R¹⁰—, —CR⁹R¹⁰—NR⁸—, —NR⁸—CR⁹R¹⁰—CR¹¹R¹²—, —CR⁹R¹⁰—NR⁸—CR¹¹R¹²—, or —CR⁹R¹⁰—CR¹¹R¹²—NR⁸—;

### X is CR¹⁸, C(R¹⁸)₂, N, or NR¹⁸;

### Y is CR¹⁸, C, or N;

### Z is O or S;

L is —(CH₂)ₚ—O—, —(CH₂)ₚ—, —CH═CH—, or a bond;

### A is C, CH, or N;

B is aryl, heteroaryl, heterocyclyl, or cycloalkyl, wherein each of the aryl, heteroaryl, heterocyclyl, or cycloalkyl is optionally substituted with from 1 to 3 substituents selected from the group consisting of H, alkoxy, —S-alkyl, optionally substituted C₁-C₆ alkyl, halogen, —CF₃, —OCF₃, and —CN;
n is 0, 1, 2, or 3;
p is from 1 to 4;
q is 0, 1, or 2;
r is from 1 to 4; and [symbol]
represents an optional double bond,
or an oxide thereof, a pharmaceutically acceptable salt thereof, a solvate thereof, or prodrug thereof.

As used above, and throughout the description herein, the following terms, unless otherwise indicated, shall be understood to have the following meanings. If not defined otherwise herein, all technical and scientific terms used herein have the same meaning as is commonly understood by one of ordinary skill in the art to which this technology belongs. In the event that there is a plurality of definitions for a term herein, those in this section prevail unless stated otherwise.

The term “alkyl” means an aliphatic hydrocarbon group which may be straight or branched. When not otherwise restricted, the term refers to an alkyl of 20 or fewer carbons. Lower alkyl refers to alkyl groups having about 1 to about 6 carbon atoms in the chain. Branched means that one or more lower alkyl groups such as methyl, ethyl or propyl are attached to a linear alkyl chain. Exemplary alkyl groups include methyl, ethyl, n-propyl, i-propyl, n-butyl, t-butyl, n-pentyl, 3-pentyl, and the like.

The term “alkenyl” means an aliphatic hydrocarbon group containing a carbon-carbon double bond and which may be straight or branched having about 2 to about 6 carbon atoms in the chain. Particular alkenyl groups have 2 to about 4 carbon atoms in the chain. Branched means that one or more lower alkyl groups such as methyl, ethyl, or propyl are attached to a linear alkenyl chain. Exemplary alkenyl groups include ethenyl, propenyl, n-butenyl, and i-butenyl. The term “alkenyl” may also refer to a hydrocarbon chain having 2 to 6 carbons containing at least one double bond and at least one triple bond.

The term “alkynyl” means an aliphatic hydrocarbon group containing a carbon-carbon triple bond and which may be straight or branched having about 2 to about 6 carbon atoms in the chain. Particular alkynyl groups have 2 to about 4 carbon atoms in the chain. Branched means that one or more lower alkyl groups such as methyl, ethyl, or propyl are attached to a linear alkynyl chain. Exemplary alkynyl groups include ethynyl, propynyl, n-butynyl, 2-butynyl, 3-methylbutynyl, and n-pentynyl.

The term “aryl” means an aromatic monocyclic or multi-cyclic (polycyclic) ring system of 6 to about 19 carbon atoms, or of 6 to about 10 carbon atoms, and includes arylalkyl groups. The ring system of the aryl group may be optionally substituted. Representative aryl groups include, but are not limited to, groups such as phenyl, naphthyl, azulenyl, phenanthrenyl, anthracenyl, fluorenyl, pyrenyl, triphenylenyl, chrysenyl, and naphthacenyl.

The term “arylalkyl” means an alkyl residue attached to an aryl ring. Examples are benzyl, phenethyl, and the like.

The term “alkoxy” means groups of from 1 to 8 carbon atoms of a straight, branched, or cyclic configuration and combinations thereof attached to the parent structure through an oxygen. Examples include methoxy, ethoxy, propoxy, isopropoxy, cyclopropyloxy, cyclohexyloxy, and the like. Lower-alkoxy refers to groups containing one to four carbons. For the purposes of the present patent application, alkoxy also includes methylenedioxy and ethylenedioxy in which each oxygen atom is bonded to the atom, chain, or ring from which the methylenedioxy or ethylenedioxy group is pendant so as to form a ring. Thus, for example, phenyl substituted by alkoxy may be, for example,

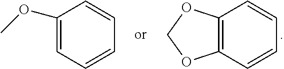

The term “compound,” “product compound,” and equivalent expressions, are meant to embrace compounds of general formula I as hereinbefore described. Also contemplated are the prodrugs, the pharmaceutically acceptable salts, the oxides, the solvates, e.g. hydrates, and inclusion complexes of that compound, where the context so permits, as well as any stereoisomeric form, or a mixture of any such forms of that compound in any ratio. Inclusion complexes are described in Remington, The Science and Practice of Pharmacy, 19th Ed. 1:176-177 (1995), which is hereby incorporated by reference in its entirety. The most commonly employed inclusion complexes are those with cyclodextrins, and all cyclodextrin complexes, natural and synthetic, are specifically encompassed within the claims. In accordance with some embodiments, a compound as described herein, including in the contexts of pharmaceutical compositions, methods of treatment, and compounds per se, is provided as the salt form. Similarly, reference to intermediates, whether or not they themselves are claimed, is meant to embrace their salts, and solvates, where the context so permits. For the sake of clarity, particular instances when the context so permits are sometimes indicated in the text, but these instances are purely illustrative and it is not intended to exclude other instances when the context so permits.

The term “cycloalkyl” means a non-aromatic, saturated or unsaturated, mono- or multi-cyclic ring system of about 3 to about 7 carbon atoms, or of about 5 to about 7 carbon atoms, and which may include at least one double bond. Exemplary cycloalkyl groups include, without limitation, cyclopropyl, cyclobutyl, cyclopentyl, cyclohexyl, cycloheptyl, cyclopropenyl, cyclobutenyl, cyclopentenyl, cyclophenyl, anti-bicyclopropane, and syn-tricyclopropane.

The term “cycloalkylalkyl” means a cycloalkyl-alkyl-group in which the cycloalkyl and alkyl are as defined herein. Exemplary cycloalkylalkyl groups include cyclopropylmethyl and cyclopentylmethyl. The alkyl radical and the cycloalkyl radical may be optionally substituted as defined herein.

The term “haloalkyl” means both branched and straight-chain alkyl substituted with one or more halogen, wherein the alkyl group is as herein described.

The term “halogen” means fluorine, chlorine, bromine, or iodine.

The term “heteroaryl” means an aromatic monocyclic or multi-cyclic ring system of about 5 to about 19 ring atoms, or about 5 to about 10 ring atoms, in which one or more of the atoms in the ring system is/are element(s) other than carbon, for example, nitrogen, oxygen, or sulfur. In the case of multi-cyclic ring system, only one of the rings needs to be aromatic for the ring system to be defined as “heteroaryl”. Particular heteroaryls contain about 5 to 6 ring atoms. The prefix aza, oxa, thia, or thio before heteroaryl means that at least a nitrogen, oxygen, or sulfur atom, respectively, is present as a ring atom. A nitrogen, carbon, or sulfur atom in the heteroaryl ring may be optionally oxidized; the nitrogen may optionally be quaternized. Representative heteroaryls include pyridyl, 2-oxo-pyridinyl, pyrimidinyl, pyridazinyl, pyrazinyl, triazinyl, furanyl, pyrrolyl, thiophenyl, pyrazolyl, imidazolyl, oxazolyl, isoxazolyl, thiazolyl, isothiazolyl, triazolyl, oxadiazolyl, thiadiazolyl, tetrazolyl, indolyl, isoindolyl, benzofuranyl, benzothiophenyl, indolinyl, 2-oxoindolinyl, dihydrobenzofuranyl, dihydrobenzothiophenyl, indazolyl, benzimidazolyl, benzooxazolyl, benzothiazolyl, benzoisoxazolyl, benzoisothiazolyl, benzotriazolyl, benzo[1,3]dioxolyl, quinolinyl, isoquinolinyl, quinazolinyl, cinnolinyl, pthalazinyl, quinoxalinyl, 2,3-dihydro-benzo[1,4]dioxinyl, benzo[1,2,3]triazinyl, benzo[1,2,4]triazinyl, 4H-chromenyl, indolizinyl, quinolizinyl, 6aH-thieno[2,3-d]imidazolyl, 1H-pyrrolo[2,3-b]pyridinyl, imidazo[1,2-a]pyridinyl, pyrazolo[1,5-a]pyridinyl, [1,2,4]triazolo[4,3-a]pyridinyl, [1,2,4]triazolo[1,5-a]pyridinyl, thieno[2,3-b]furanyl, thieno[2,3-b]pyridinyl, thieno[3,2-b]pyridinyl, furo[2,3-b]pyridinyl, furo[3,2-b]pyridinyl, thieno[3,2-d]pyrimidinyl, furo[3,2-d]pyrimidinyl, thieno[2,3-b]pyrazinyl, imidazo[1,2-a]pyrazinyl, 5,6,7,8-tetrahydroimidazo[1,2-a]pyrazinyl, 6,7-dihydro-4H-pyrazolo[5,1-c][1,4]oxazinyl, 2-oxo-2,3-dihydrobenzo[d]oxazolyl, 3,3-dimethyl-2-oxoindolinyl, 2-oxo-2,3-dihydro-1H-pyrrolo[2,3-b]pyridinyl, benzo[c][1,2,5]oxadiazolyl, benzo[c][1,2,5]thiadiazolyl, 3,4-dihydro-2H-benzo[b][1,4]oxazinyl, 5,6,7,8-tetrahydro-[1,2,4]triazolo[4,3-a]pyrazinyl, [1,2,4]triazolo[4,3-a]pyrazinyl, 3-oxo-[1,2,4]triazolo[4,3-a]pyridin-2(3H)-yl, and the like.

As used herein, “heterocyclyl” or “heterocycle” refers to a stable 3- to 18-membered ring (radical) which consists of carbon atoms and from one to five heteroatoms selected from the group consisting of nitrogen, oxygen and sulfur. For purposes of this application, the heterocycle may be a monocyclic, or a polycyclic ring system, which may include fused, bridged, or spiro ring systems; and the nitrogen, carbon, or sulfur atoms in the heterocycle may be optionally oxidized; the nitrogen atom may be optionally quaternized; and the ring may be partially or fully saturated. Examples of such heterocycles include, without limitation, azepinyl, azocanyl, pyranyl dioxanyl, dithianyl, 1,3-dioxolanyl, tetrahydrofuryl, dihydropyrrolidinyl, decahydroisoquinolyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, 2-oxoazepinyl, oxazolidinyl, oxiranyl, piperidinyl, piperazinyl, 4-piperidonyl, pyrrolidinyl, pyrazolidinyl, thiazolidinyl, tetrahydropyranyl, thiamorpholinyl, thiamorpholinyl sulfoxide, and thiamorpholinyl sulfone. Further heterocycles and heteroaryls are described in Katritzky et al., eds., Comprehensive Heterocyclic Chemistry: The Structure, Reactions, Synthesis and Use of Heterocyclic Compounds, Vol. 1-8, Pergamon Press, N.Y. (1984), which is hereby incorporated by reference in its entirety.

The term “method of treating” means amelioration or relief from the symptoms and/or effects associated with the disorders described herein. As used herein, reference to “treatment” of a patient is intended to include prophylaxis.

The term “monocyclic” used herein indicates a molecular structure having one ring.

The term “optionally substituted” is used to indicate that a group may have a substituent at each substitutable atom of the group (including more than one substituent on a single atom), provided that the designated atom's normal valency is not exceeded and the identity of each substituent is independent of the others. Up to three H atoms in each residue are replaced with alkyl, halogen, haloalkyl, hydroxy, loweralkoxy, carboxy, carboalkoxy (also referred to as alkoxycarbonyl), carboxamido (also referred to as alkylaminocarbonyl), cyano, carbonyl, nitro, amino, alkylamino, dialkylamino, mercapto, alkylthio, sulfoxide, sulfone, acylamino, amidino, phenyl, benzyl, heteroaryl, phenoxy, benzyloxy, or heteroaryloxy. “Unsubstituted” atoms bear all of the hydrogen atoms dictated by their valency. When a substituent is keto (i.e., =0), then two hydrogens on the atom are replaced. Combinations of substituents and/or variables are permissible only if such combinations result in stable compounds; by “stable compound” or “stable structure” is meant a compound that is sufficiently robust to survive isolation to a useful degree of purity from a reaction mixture, and formulation into an efficacious therapeutic agent.

The term “pharmaceutical composition” means a composition comprising a compound of formula I and at least one component comprising pharmaceutically acceptable carriers, diluents, adjuvants, excipients, or vehicles, such as preserving agents, fillers, disintegrating agents, wetting agents, emulsifying agents, suspending agents, sweetening agents, flavoring agents, perfuming agents, antibacterial agents, antifungal agents, lubricating agents and dispensing agents, depending on the nature of the mode of administration and dosage forms. As used herein, the term “pharmaceutically acceptable carrier” is used to mean any carrier, diluent, adjuvant, excipient, or vehicle, as described herein. Examples of suspending agents include ethoxylated isostearyl alcohols, polyoxyethylene sorbitol and sorbitan esters, microcrystalline cellulose, aluminum metahydroxide, bentonite, agar-agar and tragacanth, or mixtures of these substances. Prevention of the action of microorganisms can be ensured by various antibacterial and antifungal agents, for example, parabens, chlorobutanol, phenol, sorbic acid, and the like. It may also be desirable to include isotonic agents, for example sugars, sodium chloride, and the like. Prolonged absorption of the injectable pharmaceutical form can be brought about by the use of agents delaying absorption, for example, aluminum monosterate and gelatin. Examples of suitable carriers, diluents, solvents, or vehicles include water, ethanol, polyols, suitable mixtures thereof, vegetable oils (such as olive oil), and injectable organic esters such as ethyl oleate. Examples of excipients include lactose, milk sugar, sodium citrate, calcium carbonate, and dicalcium phosphate. Examples of disintegrating agents include starch, alginic acids, and certain complex silicates. Examples of lubricants include magnesium stearate, sodium lauryl sulphate, talc, as well as high molecular weight polyethylene glycols.

The term “pharmaceutically acceptable” means it is, within the scope of sound medical judgment, suitable for use in contact with the cells of humans and lower animals without undue toxicity, irritation, allergic response and the like, and are commensurate with a reasonable benefit/risk ratio.

The term “pharmaceutically acceptable dosage forms” means dosage forms of the compounds described herein, and includes, for example, tablets, dragees, powders, elixirs, syrups, liquid preparations, including suspensions, sprays, inhalants tablets, lozenges, emulsions, solutions, granules, capsules, and suppositories, as well as liquid preparations for injections, including liposome preparations. Techniques and formulations generally may be found in Remington's Pharmaceutical Sciences, Mack Publishing Co., Easton, Pa., latest edition, which is hereby incorporated by reference in its entirety.

The term “pharmaceutically acceptable prodrugs” as used herein means those prodrugs of the compounds useful as described herein which are, within the scope of sound medical judgment, suitable for use in contact with the tissues of humans and lower animals with undue toxicity, irritation, allergic response, and the like, commensurate with a reasonable benefit/risk ratio, and effective for their intended use, as well as the zwitterionic forms, where possible. The term “prodrug” means compounds that are rapidly transformed in vivo to yield the parent compound of the above formula, for example by hydrolysis in blood. Commonly, the conversion of prodrug to drug occurs by enzymatic processes in the liver or blood of the mammal. Many of the compounds described herein may be chemically modified without absorption into the systemic circulation, and in those cases, activation in vivo may come about by chemical action (as in the acid-catalyzed cleavage in the stomach) or through the intermediacy of enzymes and microflora in the gastrointestinal GI tract. Functional groups which may be rapidly transformed, by metabolic cleavage, in vivo form a class of groups reactive with the carboxyl group of the compounds described herein. They include, but are not limited to, such groups as alkanoyl (such as acetyl, propionyl, butyryl, and the like), unsubstituted and substituted aroyl (such as benzoyl and substituted benzoyl), alkoxycarbonyl (such as ethoxycarbonyl), trialkylsilyl (such as trimethyl- and triethysilyl), monoesters formed with dicarboxylic acids (such as succinyl), and the like. Because of the ease with which the metabolically cleavable groups of the compounds useful as described herein are cleaved in vivo, the compounds bearing such groups act as pro-drugs. The compounds bearing the metabolically cleavable groups have the advantage that they may exhibit improved bioavailability as a result of enhanced solubility and/or rate of absorption conferred upon the parent compound by virtue of the presence of the metabolically cleavable group. A thorough discussion of prodrugs is provided in the following: Design of Prodrugs, H. Bundgaard, ed., Elsevier (1985); Methods in Enzymology, K. Widder et al, Ed., Academic Press, 42, p. 309-396 (1985); A Textbook of Drug Design and Development, Krogsgaard-Larsen and H. Bundgaard, ed., Chapter 5; “Design and Applications of Prodrugs,” p. 113-191 (1991); Advanced Drug Delivery Reviews, H. Bundgaard, 8, p. 1-38 (1992); Journal of Pharmaceutical Sciences, 77:285 (1988); Nakeya et al, Chem. Pharm. Bull., 32:692 (1984); Higuchi et al., “Pro-drugs as Novel Delivery Systems,” Vol. 14 of the A.C.S. Symposium Series, and Bioreversible Carriers in Drug Design, Edward B. Roche, ed., American Pharmaceutical Association and Pergamon Press (1987), which are incorporated herein by reference in their entirety. Examples of prodrugs include, but are not limited to, acetate, formate, and benzoate derivatives of alcohol and amine functional groups in the compounds described herein.

The term “pharmaceutically acceptable salt” refers to salts prepared from pharmaceutically acceptable non-toxic acids or bases including inorganic acids and bases and organic acids and bases. Suitable pharmaceutically acceptable acid addition salts for the compounds described herein include acetic, benzenesulfonic (besylate), benzoic, camphorsulfonic, citric, ethenesulfonic, fumaric, gluconic, glutamic, hydrobromic, hydrochloric, isethionic, lactic, maleic, malic, mandelic, methanesulfonic, mucic, nitric, pamoic, pantothenic, phosphoric, succinic, sulfuric, tartaric acid, p-toluenesulfonic, and the like. When the compounds contain an acidic side chain, suitable pharmaceutically acceptable base addition salts for the compounds described herein include metallic salts made from aluminum, calcium, lithium, magnesium, potassium, sodium and zinc or organic salts made from lysine, N,N′-dibenzylethylenediamine, chloroprocaine, choline, diethanolamine, ethylenediamine, meglumine (N-methylglucamine), and procaine. Pharmaceutically acceptable salts include, but are not limited to, amine salts, such as but not limited to N,N′dibenzylethylenediamine, chloroprocaine, choline, ammonia, diethanolamine and other hydroxyalkylamines, ethylenediamine, N-methylglucamine, procaine, N-benzylphenethylamine, 1-para-chlorobenzyl-2-pyrrolidin-1′-ylmethyl-benzimidazole, diethylamine and other alkylamines, piperazine, and tris(hydroxymethyl)aminomethane; alkali metal salts, such as but not limited to lithium, potassium, and sodium; alkali earth metal salts, such as but not limited to barium, calcium, and magnesium; transition metal salts, such as but not limited to zinc; and other metal salts, such as but not limited to sodium hydrogen phosphate and disodium phosphate; and also including, but not limited to, salts of mineral acids, such as but not limited to hydrochlorides and sulfates; and salts of organic acids, such as but not limited to acetates, lactates, malates, tartrates, citrates, ascorbates, succinates, butyrates, valerates and fumarates. Pharmaceutically acceptable esters include, but are not limited to, alkyl, alkenyl, alkynyl, aryl, heteroaryl, cycloalkyl and heterocyclyl esters of acidic groups, including, but not limited to, carboxylic acids, phosphoric acids, phosphinic acids, sulfonic acids, sulfinic acids, and boronic acids. Pharmaceutical acceptable enol ethers include, but are not limited to, derivatives of formula C═C(OR) where R is hydrogen, alkyl, alkenyl, alkynyl, aryl, heteroaryl, cycloalkyl, or heterocyclyl. Pharmaceutically acceptable enol esters include, but are not limited to, derivatives of formula C═C(OC(O) R) where R is hydrogen, alkyl, alkenyl, alkynyl, aryl, heteroaryl, cycloalkyl, or heterocyclyl. Pharmaceutical acceptable solvates and hydrates are complexes of a compound with one or more solvent or water molecules, or 1 to about 100, or 1 to about 10, or one to about 2, 3 or 4, solvent or water molecules.

The term “polycyclic” or “multi-cyclic” used herein indicates a molecular structure having two or more rings, including, but not limited to, fused, bridged, or spiro rings.

Terminology related to “protecting”, “deprotecting,” and “protected” functionalities occurs throughout this application. Such terminology is well understood by persons of skill in the art and is used in the context of processes which involve sequential treatment with a series of reagents. In that context, a protecting group refers to a group which is used to mask a functionality during a process step in which it would otherwise react, but in which reaction is undesirable. The protecting group prevents reaction at that step, but may be subsequently removed to expose the original functionality. The removal or “deprotection” occurs after the completion of the reaction or reactions in which the functionality would interfere. Thus, when a sequence of reagents is specified, as it is in the processes described herein, the person of ordinary skill can readily envision those groups that would be suitable as “protecting groups.” Suitable groups for that purpose are discussed in standard textbooks in the field of chemistry, such as Greene, Protective Groups in Organic Synthesis, John Wiley & Sons, New York (1991), which is hereby incorporated by reference in its entirety.

The term “solvate” refers to a compound of formula I in the solid state, wherein molecules of a suitable solvent are incorporated in the crystal lattice. A suitable solvent for therapeutic administration is physiologically tolerable at the dosage administered. Examples of suitable solvents for therapeutic administration are ethanol and water. When water is the solvent, the solvate is referred to as a hydrate. In general, solvates are formed by dissolving the compound in the appropriate solvent and isolating the solvate by cooling or using an antisolvent. The solvate is typically dried or azeotroped under ambient conditions.

The term “therapeutically effective amount” is meant to describe an amount of compound described herein effective in producing the desired therapeutic effect. Such amounts generally vary according to a number of factors well within the purview of ordinarily skilled artisans given the description provided herein to determine and account for. These include, without limitation: the particular subject, as well as its age, weight, height, general physical condition, and medical history, the particular compound used, as well as the carrier in which it is formulated and the route of administration selected for it; and, the nature and severity of the condition being treated.

Compounds described herein may contain one or more asymmetric centers and may thus give rise to enantiomers, diastereomers, and other stereoisomeric forms. Each chiral center may be defined, in terms of absolute stereochemistry, as (R)- or (S)-. This technology is meant to include all such possible isomers, as well as mixtures thereof, including racemic and optically pure forms. Optically active (R)- and (S)-, (−)- and (+)-, or (D)- and (L)-isomers may be prepared using chiral synthons or chiral reagents, or resolved using conventional techniques. When the compounds described herein contain olefinic double bonds or other centers of geometric asymmetry, and unless specified otherwise, it is intended that the compounds include both E and Z geometric isomers. Likewise, all tautomeric forms are also intended to be included.

This technology also envisions the “quaternization” of any basic nitrogen-containing groups of the compounds disclosed herein. The basic nitrogen can be quaternized with any agents known to those of ordinary skill in the art including, for example, lower alkyl halides, such as methyl, ethyl, propyl and butyl chloride, bromides and iodides; dialkyl sulfates including dimethyl, diethyl, dibutyl and diamyl sulfates; long chain halides such as decyl, lauryl, myristyl and stearyl chlorides, bromides and iodides; and aralkyl halides including benzyl and phenethyl bromides. Water or oil-soluble or dispersible products may be obtained by such quaternization.

In the characterization of some of the substituents, it is recited that certain substituents may combine to form rings. Unless stated otherwise, it is intended that such rings may exhibit various degrees of unsaturation (from fully saturated to fully unsaturated), may include heteroatoms and may be substituted with lower alkyl or alkoxy.

In accordance with one embodiment, R²-R⁵ are each independently selected from the group consisting of H and optionally substituted C₁-C₆ alkyl.

In accordance with one embodiment, R² and R³ or R⁴ and R⁵ combine to form an oxo group (carbonyl).

In accordance with one embodiment, R⁶ is H, halogen, or optionally substituted C₁-C₆ alkyl. In one particular embodiment, R⁶ is H.

In accordance with one embodiment, R⁷ is H, halogen, or optionally substituted C₁-C₆ alkyl.

In accordance with one embodiment, R⁸ is H. In accordance with another embodiment, R⁸ is optionally substituted C₁-C₆ alkyl, for example, methyl, ethyl, and 2-isopropyl. In accordance with yet another embodiment, R⁸ is —C(O)R¹⁴

In accordance with one embodiment, R⁸ and one of R², R³, R⁴, and R⁵ combine to form an optionally substituted 3- to 7-membered heterocycle, wherein the 3- to 7-membered heterocycle includes from 1 to 2 heteroatoms selected from the group consisting of N, O, and S. In one particular embodiment, R⁸ and one of R², R³, R⁴, and R⁵ combine to form an optionally substituted fused 5-membered heterocycle.

In accordance with one embodiment, any one of R², R³, R⁴, or R⁵ can combine with any one of R⁹, R¹⁰, R¹¹, or R¹² to form —(CH₂)ᵣ— and r is from 1 to 4.

In accordance with one embodiment, X is N, CH, or CH₂.

In accordance with one embodiment, Y is N or C.

In accordance with one embodiment, L is a bond. In accordance with another embodiment, L is —CH₂—O—. In accordance with a further embodiment, L is —(CH₂)₂—.

In accordance with one embodiment, B is aryl. In one particular embodiment, B is phenyl. In accordance with another embodiment, B is heteroaryl. In one particular embodiment, B is pyridinyl, for example pyridin-2-yl or pyridin-3-yl. Other examples of heteroaryls include, for example, pyridazinyl (e.g., pyridazin-3-yl) and pyrimidinyl (e.g., pyrimidin-5-yl).

As described herein, B may be optionally substituted. In one embodiment, B is unsubstituted. In another embodiment, B is substituted with one substituent selected from trifluoromethyl, —OCF₃, chloro, fluoro, and methyl. In accordance with one embodiment, B is selected from the group consisting of phenyl, 4-(trifluoromethyl)phenyl, pyridin-2-yl, 5-(trifluoromethyl)pyridin-2-yl, 5-fluoro-pyridin-2-yl, 6-methylpyridin-3-yl, 6-(trifluoromethyl)pyridin-2-yl, 6-(trifluoromethyl)pyridin-3-yl, 5-chloro-pyridin-2-yl, and 4-chloro-phenyl. In accordance with another embodiment, B is selected from the group consisting of 6-(trifluoromethyl)pyridazin-3-yl, 2-fluoro-4-methoxyphenyl, 2-chloro-4-methoxyphenyl, and 2-methyl-4-(trifluoromethoxy)phenyl.

Within these embodiments, the selection of a particular substituent at any one of R¹-R¹⁸, X, Y, Z, L, A, and B does not affect the selection of a substituent at any of the others of R¹-R¹⁸, X, Y, Z, L, A, and B. That is, compounds provided herein have any of the substituents at any of the positions.

In accordance with one embodiment, the compound has the structure:

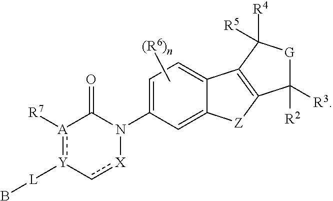

In accordance with one embodiment of the present invention, the compound has the structure

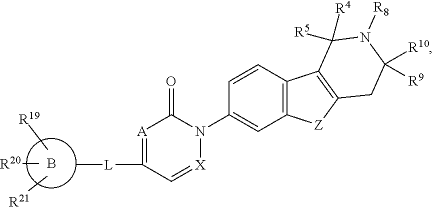

wherein R¹⁹, R²⁰, and R²¹ are individually selected from the group consisting of H, alkoxy, S-alkyl, optionally substituted C₁-C₆ alkyl, halogen, —CF₃ and —CN.

In accordance with another embodiment of the present invention, the compound has the structure

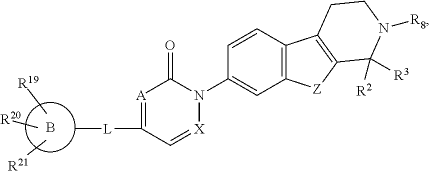

wherein R¹⁹, R²⁰, and R²¹ are individually selected from the group consisting of H, alkoxy, S-alkyl, optionally substituted C₁-C₆ alkyl, halogen, —CF₃ and —CN.

In accordance with another embodiment of the present invention, the compound has the structure

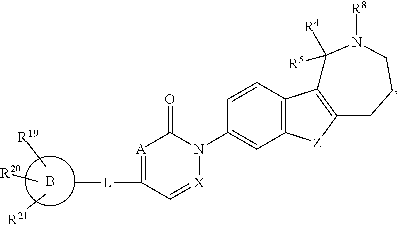

wherein R¹⁹, R²⁰, and R²¹ are individually selected from the group consisting of H, alkoxy, S-alkyl, optionally substituted C₁-C₆ alkyl, halogen, —CF₃ and —CN.

In accordance with another embodiment of the present invention, the compound has the structure

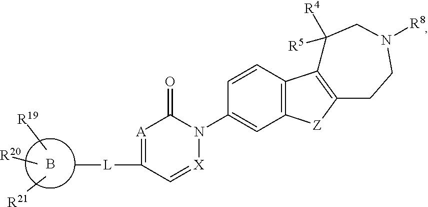

wherein R¹⁹, R²⁰, and R²¹ are individually selected from the group consisting of H, alkoxy, S-alkyl, optionally substituted C₁-C₆ alkyl, halogen, —CF₃ and —CN.

In accordance with a further embodiment of the present invention, the compound has the structure

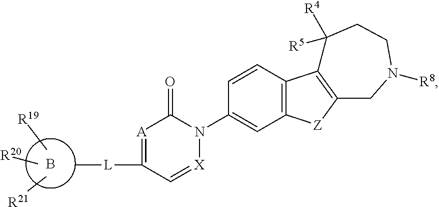

wherein R¹⁹, R²⁰, and R²¹ are individually selected from the group consisting of H, alkoxy, S-alkyl, optionally substituted C₁-C₆ alkyl, halogen, —CF₃ and —CN.

In accordance with one embodiment, the compound is selected from

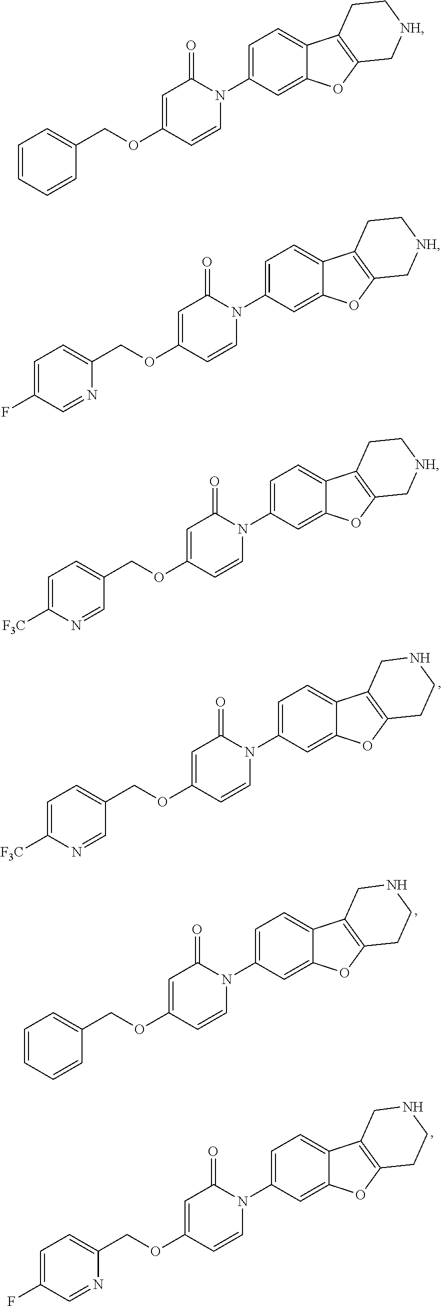 [SMILES: O=c1cc(OCc2ccccc2)ccn1-c1ccc2c3c(oc2c1)CNCC3 ; O=c1cc(OCc2ccc(F)cn2)ccn1-c1ccc2c3c(oc2c1)CNCC3 ; O=c1cc(OCc2ccc(C(F)(F)F)nc2)ccn1-c1ccc2c3c(oc2c1)CNCC3 ; O=c1cc(OCc2ccc(C(F)(F)F)nc2)ccn1-c1ccc2c3c(oc2c1)CCNC3 ; O=c1cc(OCc2ccccc2)ccn1-c1ccc2c3c(oc2c1)CCNC3 ; O=c1cc(OCc2ccc(F)cn2)ccn1-c1ccc2c3c(oc2c1)CCNC3]

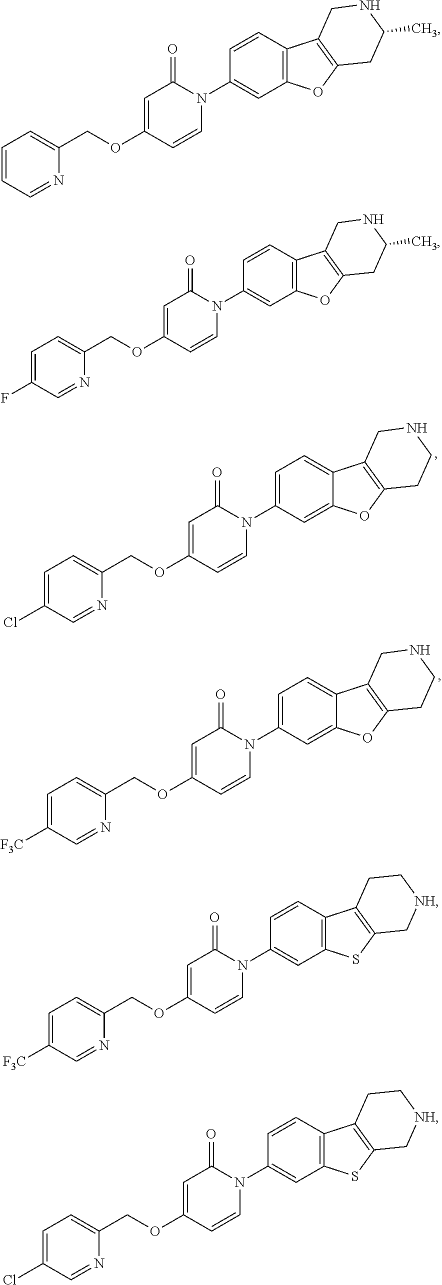 [SMILES: C[C@@H]1Cc2oc3cc(-n4ccc(OCc5ccccn5)cc4=O)ccc3c2CN1 ; C[C@@H]1Cc2oc3cc(-n4ccc(OCc5ccc(F)cn5)cc4=O)ccc3c2CN1 ; O=c1cc(OCc2ccc(Cl)cn2)ccn1-c1ccc2c3c(oc2c1)CCNC3 ; O=c1cc(OCc2ccc(C(F)(F)F)cn2)ccn1-c1ccc2c3c(oc2c1)CCNC3 ; O=c1cc(OCc2ccc(C(F)(F)F)cn2)ccn1-c1ccc2c3c(sc2c1)CNCC3 ; O=c1cc(OCc2ccc(Cl)cn2)ccn1-c1ccc2c3c(sc2c1)CNCC3]

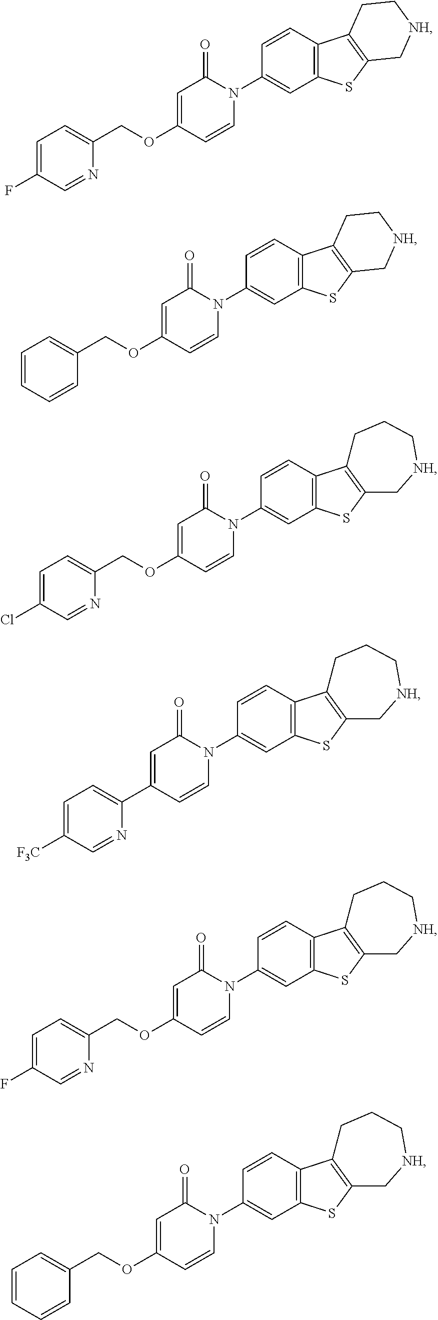 [SMILES: O=c1cc(OCc2ccc(F)cn2)ccn1-c1ccc2c3c(sc2c1)CNCC3 ; O=c1cc(OCc2ccccc2)ccn1-c1ccc2c3c(sc2c1)CNCC3 ; O=c1cc(OCc2ccc(Cl)cn2)ccn1-c1ccc2c3c(sc2c1)CNCCC3 ; O=c1cc(-c2ccc(C(F)(F)F)cn2)ccn1-c1ccc2c3c(sc2c1)CNCCC3 ; O=c1cc(OCc2ccc(F)cn2)ccn1-c1ccc2c3c(sc2c1)CNCCC3 ; O=c1cc(OCc2ccccc2)ccn1-c1ccc2c3c(sc2c1)CNCCC3]

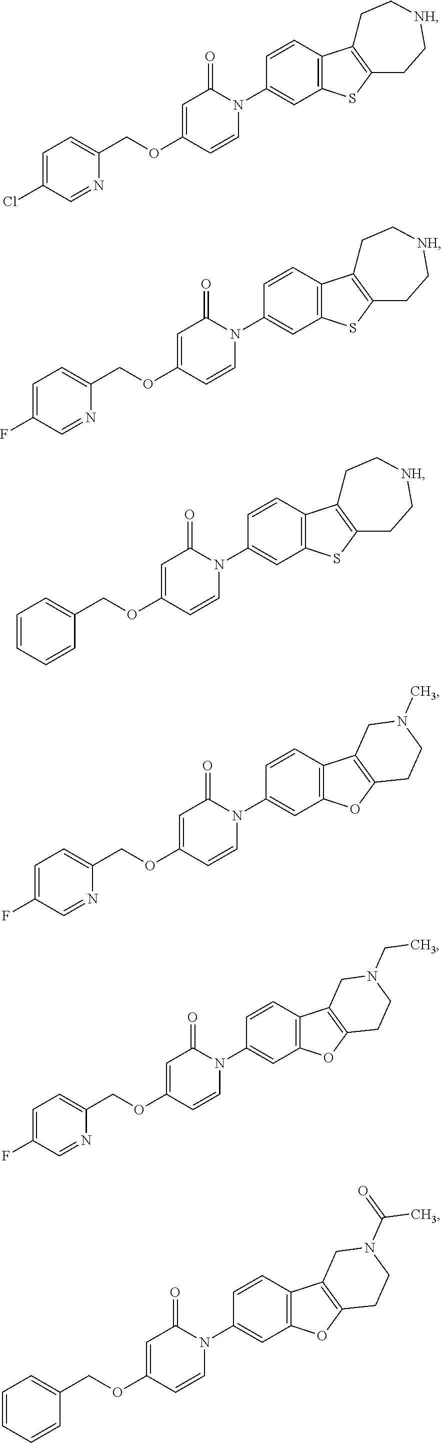 [SMILES: O=c1cc(OCc2ccc(Cl)cn2)ccn1-c1ccc2c3c(sc2c1)CCNCC3 ; O=c1cc(OCc2ccc(F)cn2)ccn1-c1ccc2c3c(sc2c1)CCNCC3 ; O=c1cc(OCc2ccccc2)ccn1-c1ccc2c3c(sc2c1)CCNCC3 ; CN1CCc2oc3cc(-n4ccc(OCc5ccc(F)cn5)cc4=O)ccc3c2C1 ; CCN1CCc2oc3cc(-n4ccc(OCc5ccc(F)cn5)cc4=O)ccc3c2C1 ; CC(=O)N1CCc2oc3cc(-n4ccc(OCc5ccccc5)cc4=O)ccc3c2C1]

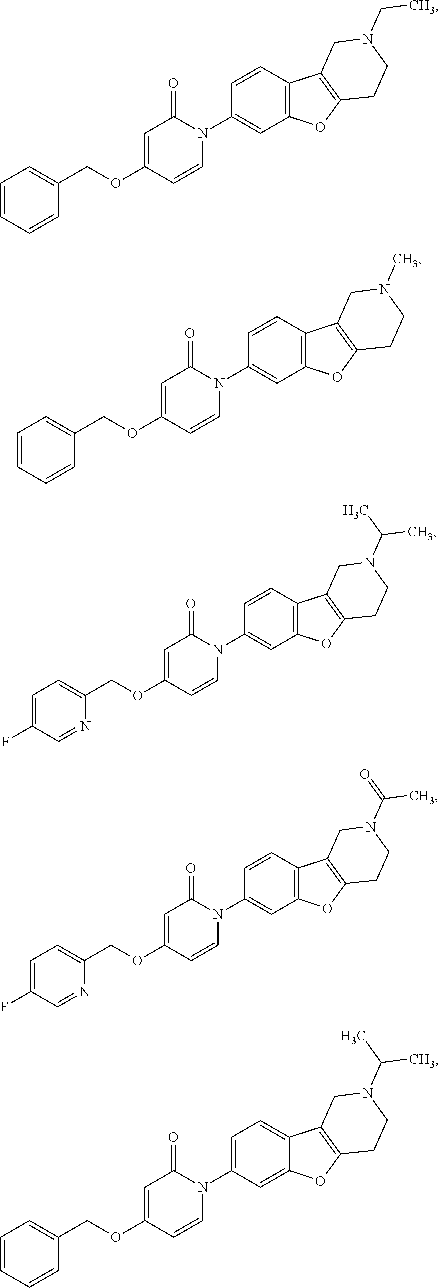 [SMILES: CCN1CCc2oc3cc(-n4ccc(OCc5ccccc5)cc4=O)ccc3c2C1 ; CN1CCc2oc3cc(-n4ccc(OCc5ccccc5)cc4=O)ccc3c2C1 ; CC(C)N1CCc2oc3cc(-n4ccc(OCc5ccc(F)cn5)cc4=O)ccc3c2C1 ; CC(=O)N1CCc2oc3cc(-n4ccc(OCc5ccc(F)cn5)cc4=O)ccc3c2C1 ; CC(C)N1CCc2oc3cc(-n4ccc(OCc5ccccc5)cc4=O)ccc3c2C1]

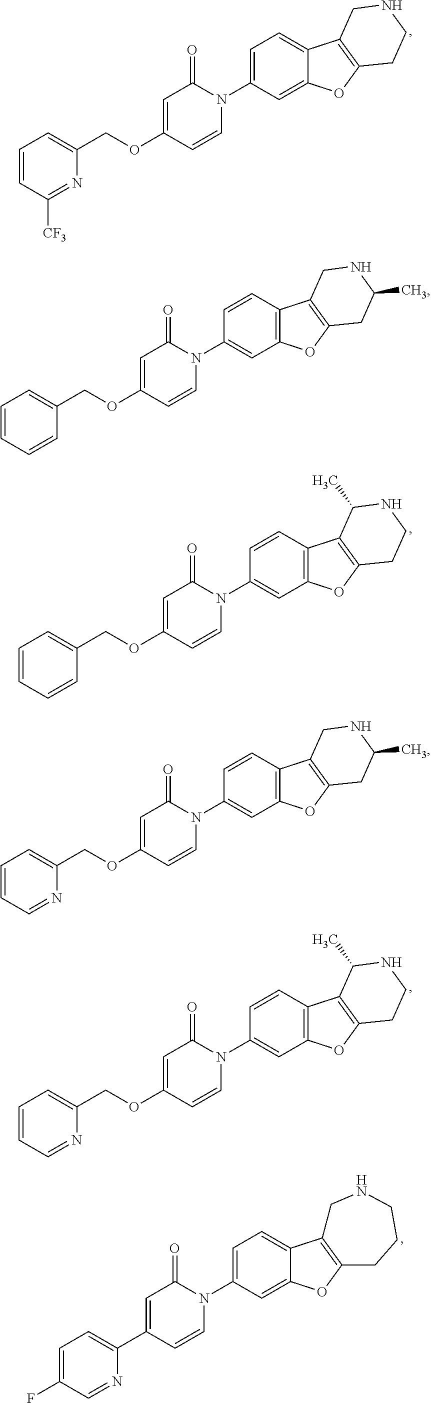 [SMILES: O=c1cc(OCc2cccc(C(F)(F)F)n2)ccn1-c1ccc2c3c(oc2c1)CCNC3 ; C[C@H]1Cc2oc3cc(-n4ccc(OCc5ccccc5)cc4=O)ccc3c2CN1 ; C[C@@H]1NCCc2oc3cc(-n4ccc(OCc5ccccc5)cc4=O)ccc3c21 ; C[C@H]1Cc2oc3cc(-n4ccc(OCc5ccccn5)cc4=O)ccc3c2CN1 ; C[C@@H]1NCCc2oc3cc(-n4ccc(OCc5ccccn5)cc4=O)ccc3c21 ; O=c1cc(-c2ccc(F)cn2)ccn1-c1ccc2c3c(oc2c1)CCCNC3]

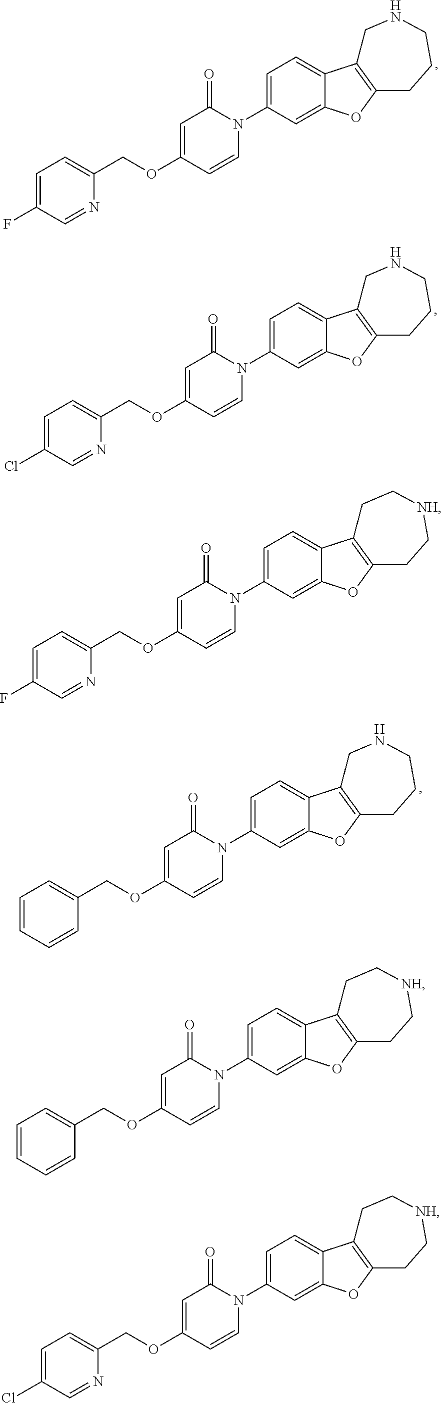 [SMILES: O=c1cc(OCc2ccc(F)cn2)ccn1-c1ccc2c3c(oc2c1)CCCNC3 ; O=c1cc(OCc2ccc(Cl)cn2)ccn1-c1ccc2c3c(oc2c1)CCCNC3 ; O=c1cc(OCc2ccc(F)cn2)ccn1-c1ccc2c3c(oc2c1)CCNCC3 ; O=c1cc(OCc2ccccc2)ccn1-c1ccc2c3c(oc2c1)CCCNC3 ; O=c1cc(OCc2ccccc2)ccn1-c1ccc2c3c(oc2c1)CCNCC3 ; O=c1cc(OCc2ccc(Cl)cn2)ccn1-c1ccc2c3c(oc2c1)CCNCC3]

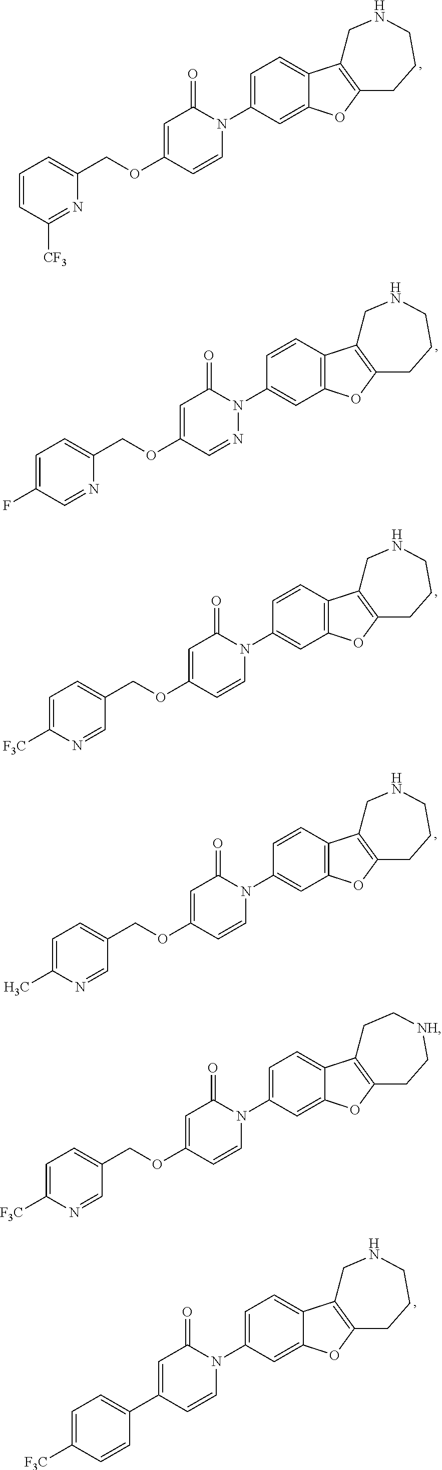 [SMILES: O=c1cc(OCc2cccc(C(F)(F)F)n2)ccn1-c1ccc2c3c(oc2c1)CCCNC3 ; O=c1cc(OCc2ccc(F)cn2)cnn1-c1ccc2c3c(oc2c1)CCCNC3 ; O=c1cc(OCc2ccc(C(F)(F)F)nc2)ccn1-c1ccc2c3c(oc2c1)CCCNC3 ; Cc1ccc(COc2ccn(-c3ccc4c5c(oc4c3)CCCNC5)c(=O)c2)cn1 ; O=c1cc(OCc2ccc(C(F)(F)F)nc2)ccn1-c1ccc2c3c(oc2c1)CCNCC3 ; O=c1cc(-c2ccc(C(F)(F)F)cc2)ccn1-c1ccc2c3c(oc2c1)CCCNC3]

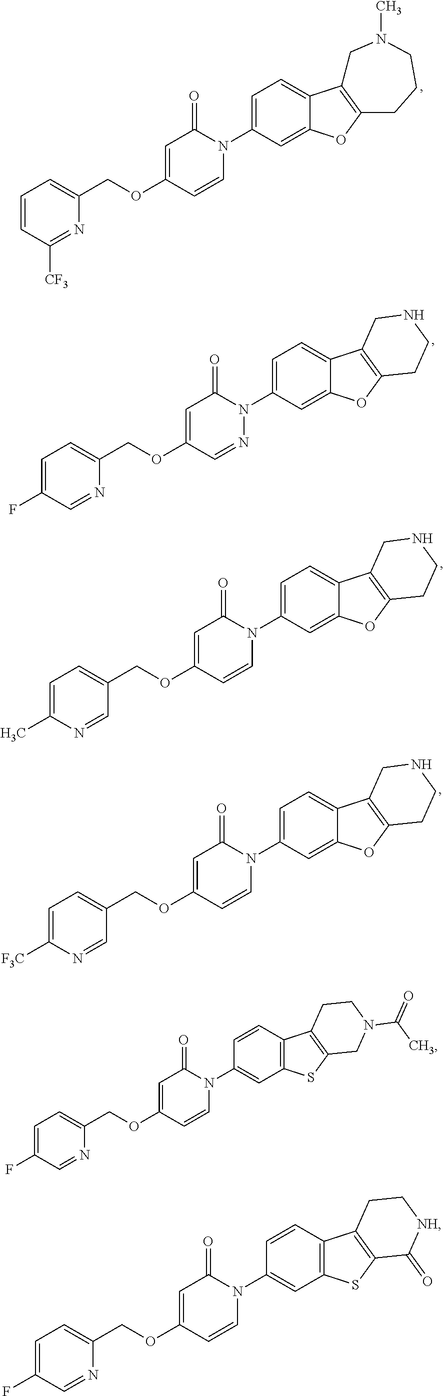 [SMILES: CN1CCCc2oc3cc(-n4ccc(OCc5cccc(C(F)(F)F)n5)cc4=O)ccc3c2C1 ; O=c1cc(OCc2ccc(F)cn2)cnn1-c1ccc2c3c(oc2c1)CCNC3 ; Cc1ccc(COc2ccn(-c3ccc4c5c(oc4c3)CCNC5)c(=O)c2)cn1 ; O=c1cc(OCc2ccc(C(F)(F)F)nc2)ccn1-c1ccc2c3c(oc2c1)CCNC3 ; CC(=O)N1CCc2c(sc3cc(-n4ccc(OCc5ccc(F)cn5)cc4=O)ccc23)C1 ; O=C1NCCc2c1sc1cc(-n3ccc(OCc4ccc(F)cn4)cc3=O)ccc21]

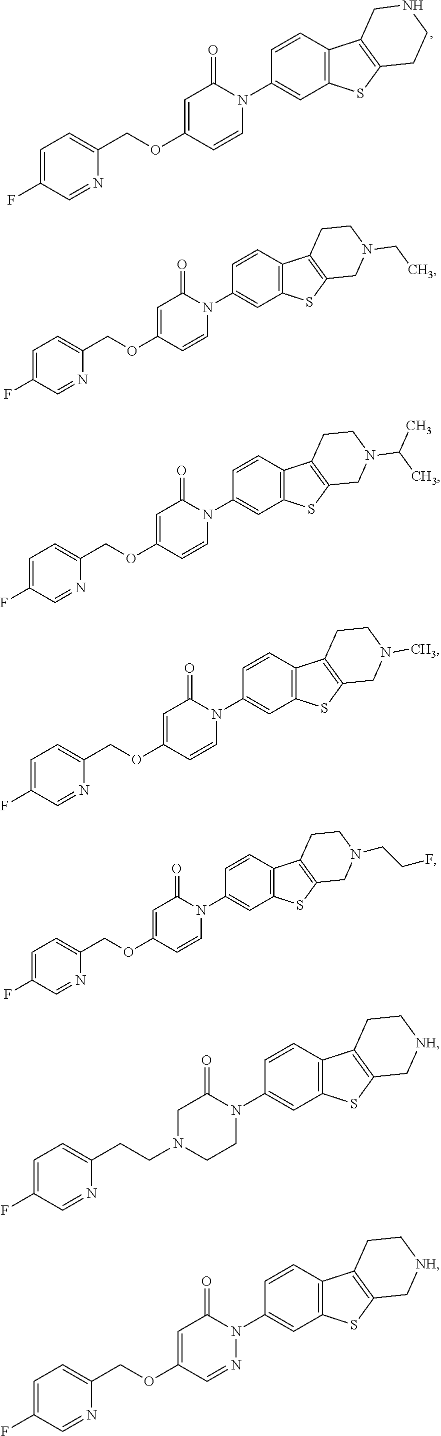

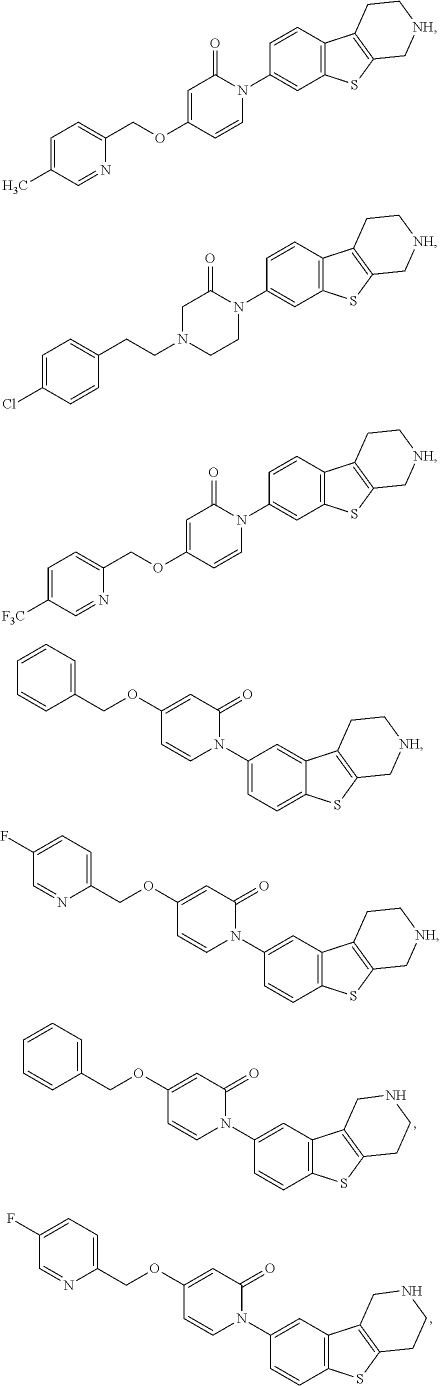

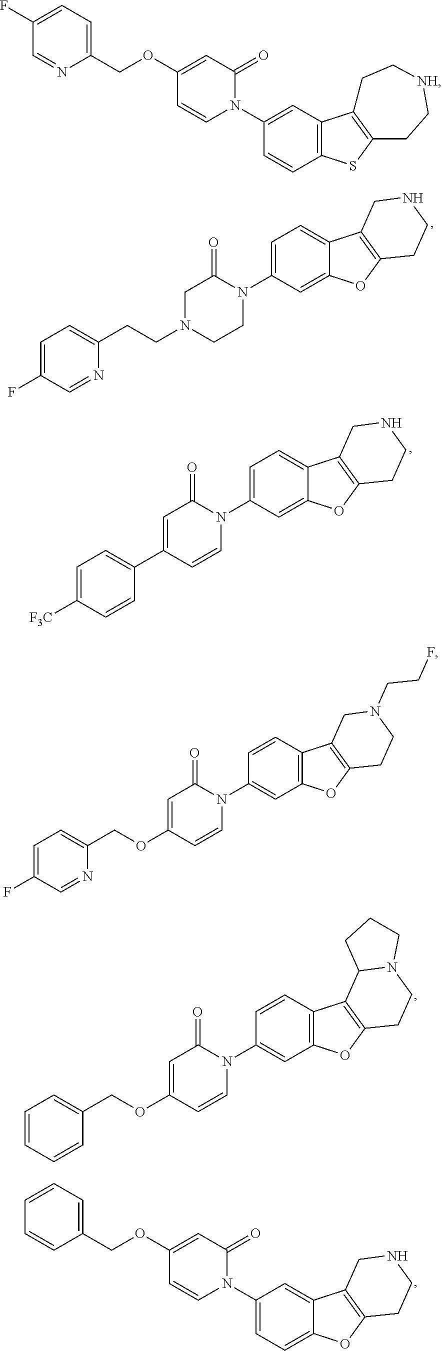

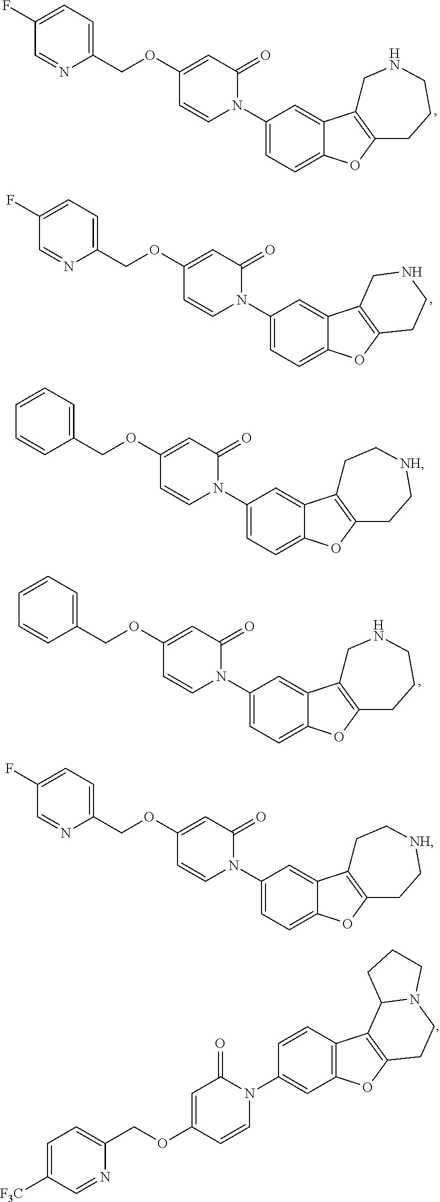 [SMILES: O=c1cc(OCc2ccc(F)cn2)ccn1-c1ccc2oc3c(c2c1)CNCCC3 ; O=c1cc(OCc2ccc(F)cn2)ccn1-c1ccc2oc3c(c2c1)CNCC3 ; O=c1cc(OCc2ccccc2)ccn1-c1ccc2oc3c(c2c1)CCNCC3 ; O=c1cc(OCc2ccccc2)ccn1-c1ccc2oc3c(c2c1)CNCCC3 ; O=c1cc(OCc2ccc(F)cn2)ccn1-c1ccc2oc3c(c2c1)CCNCC3 ; O=c1cc(OCc2ccc(C(F)(F)F)cn2)ccn1-c1ccc2c3c(oc2c1)CCN1CCCC31]

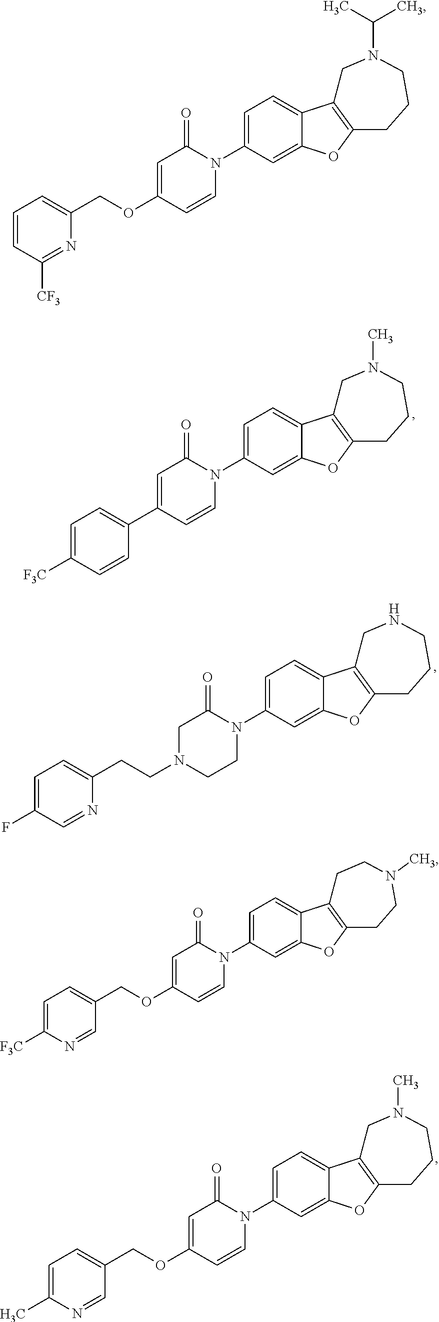 [SMILES: CC(C)N1CCCc2oc3cc(-n4ccc(OCc5cccc(C(F)(F)F)n5)cc4=O)ccc3c2C1 ; CN1CCCc2oc3cc(-n4ccc(-c5ccc(C(F)(F)F)cc5)cc4=O)ccc3c2C1 ; O=C1CN(CCc2ccc(F)cn2)CCN1c1ccc2c3c(oc2c1)CCCNC3 ; CN1CCc2oc3cc(-n4ccc(OCc5ccc(C(F)(F)F)nc5)cc4=O)ccc3c2CC1 ; Cc1ccc(COc2ccn(-c3ccc4c5c(oc4c3)CCCN(C)C5)c(=O)c2)cn1]

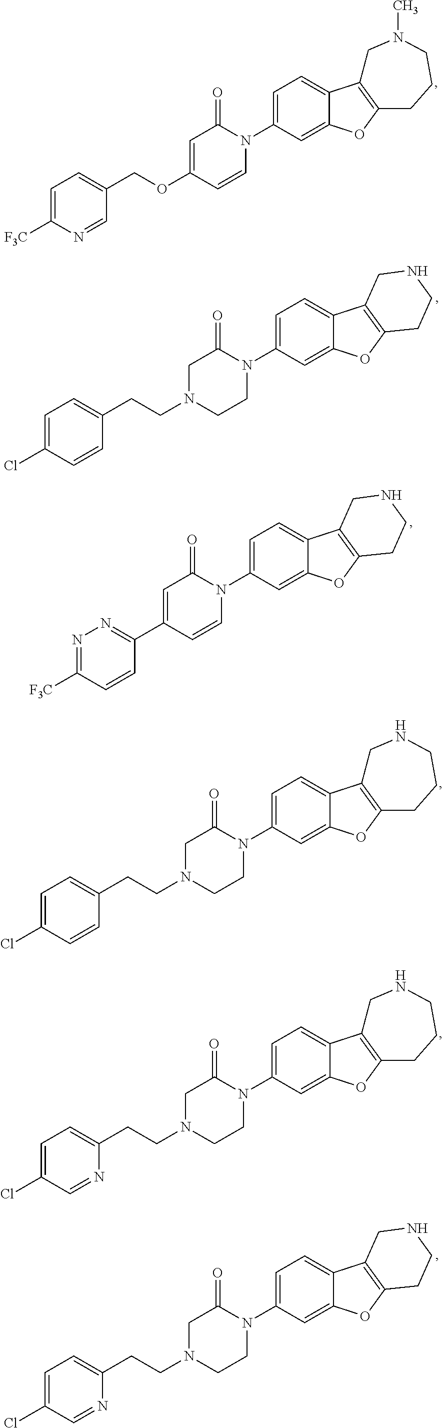 [SMILES: CN1CCCc2oc3cc(-n4ccc(OCc5ccc(C(F)(F)F)nc5)cc4=O)ccc3c2C1 ; O=C1CN(CCc2ccc(Cl)cc2)CCN1c1ccc2c3c(oc2c1)CCNC3 ; O=c1cc(-c2ccc(C(F)(F)F)nn2)ccn1-c1ccc2c3c(oc2c1)CCNC3 ; O=C1CN(CCc2ccc(Cl)cc2)CCN1c1ccc2c3c(oc2c1)CCCNC3 ; O=C1CN(CCc2ccc(Cl)cn2)CCN1c1ccc2c3c(oc2c1)CCCNC3 ; O=C1CN(CCc2ccc(Cl)cn2)CCN1c1ccc2c3c(oc2c1)CCNC3]

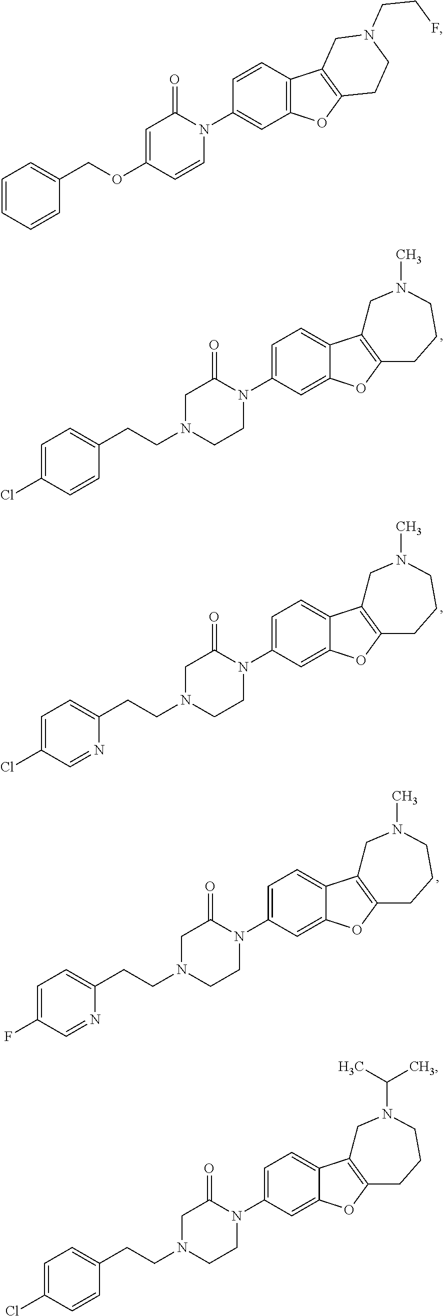 [SMILES: O=c1cc(OCc2ccccc2)ccn1-c1ccc2c3c(oc2c1)CCN(CCF)C3 ; CN1CCCc2oc3cc(N4CCN(CCc5ccc(Cl)cc5)CC4=O)ccc3c2C1 ; CN1CCCc2oc3cc(N4CCN(CCc5ccc(Cl)cn5)CC4=O)ccc3c2C1 ; CN1CCCc2oc3cc(N4CCN(CCc5ccc(F)cn5)CC4=O)ccc3c2C1 ; CC(C)N1CCCc2oc3cc(N4CCN(CCc5ccc(Cl)cc5)CC4=O)ccc3c2C1]

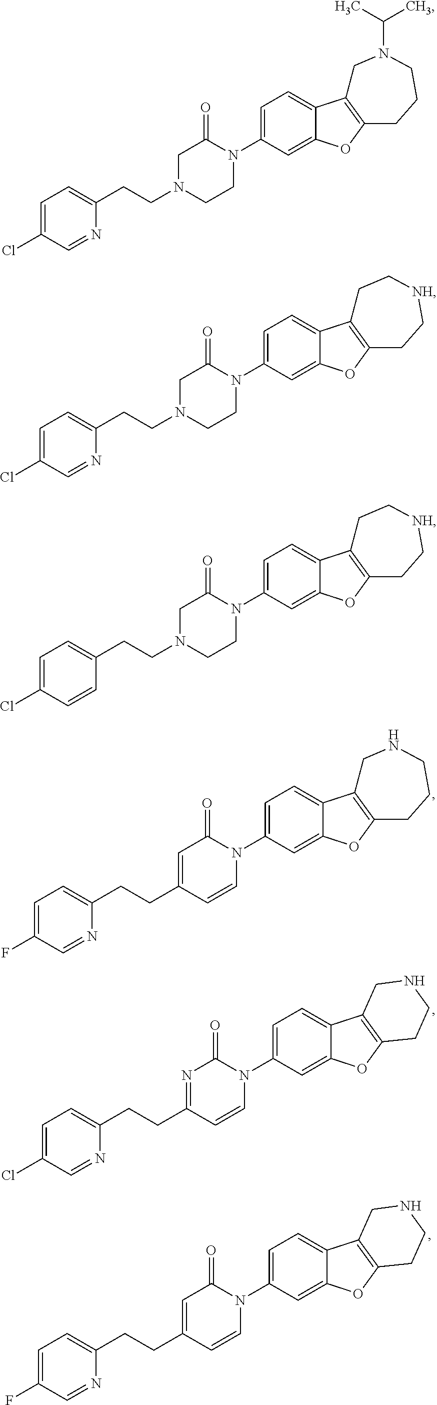 [SMILES: CC(C)N1CCCc2oc3cc(N4CCN(CCc5ccc(Cl)cn5)CC4=O)ccc3c2C1 ; O=C1CN(CCc2ccc(Cl)cn2)CCN1c1ccc2c3c(oc2c1)CCNCC3 ; O=C1CN(CCc2ccc(Cl)cc2)CCN1c1ccc2c3c(oc2c1)CCNCC3 ; O=c1cc(CCc2ccc(F)cn2)ccn1-c1ccc2c3c(oc2c1)CCCNC3 ; O=c1nc(CCc2ccc(Cl)cn2)ccn1-c1ccc2c3c(oc2c1)CCNC3 ; O=c1cc(CCc2ccc(F)cn2)ccn1-c1ccc2c3c(oc2c1)CCNC3]

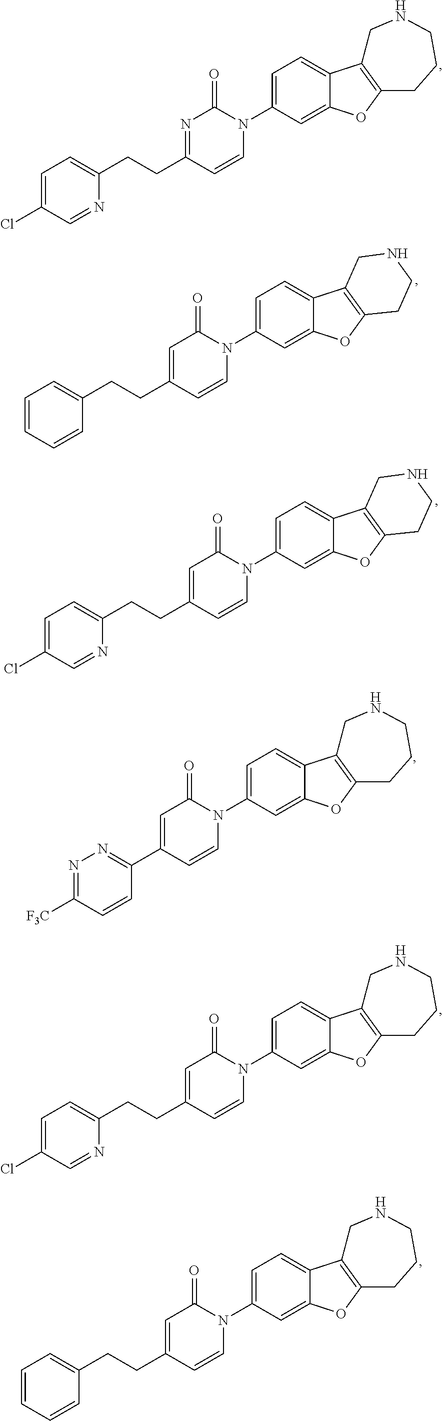 [SMILES: O=c1nc(CCc2ccc(Cl)cn2)ccn1-c1ccc2c3c(oc2c1)CCCNC3 ; O=c1cc(CCc2ccccc2)ccn1-c1ccc2c3c(oc2c1)CCNC3 ; O=c1cc(CCc2ccc(Cl)cn2)ccn1-c1ccc2c3c(oc2c1)CCNC3 ; O=c1cc(-c2ccc(C(F)(F)F)nn2)ccn1-c1ccc2c3c(oc2c1)CCCNC3 ; O=c1cc(CCc2ccc(Cl)cn2)ccn1-c1ccc2c3c(oc2c1)CCCNC3 ; O=c1cc(CCc2ccccc2)ccn1-c1ccc2c3c(oc2c1)CCCNC3]

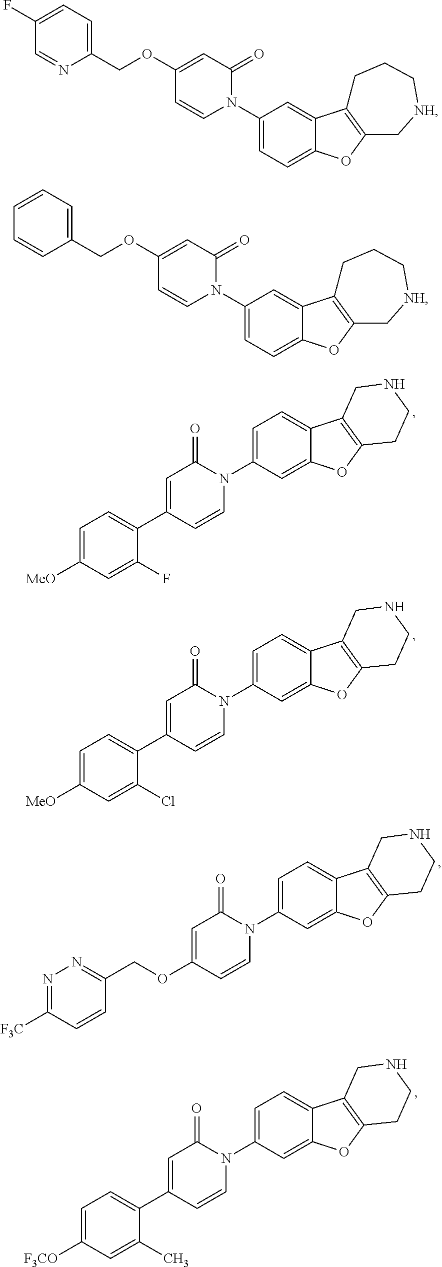

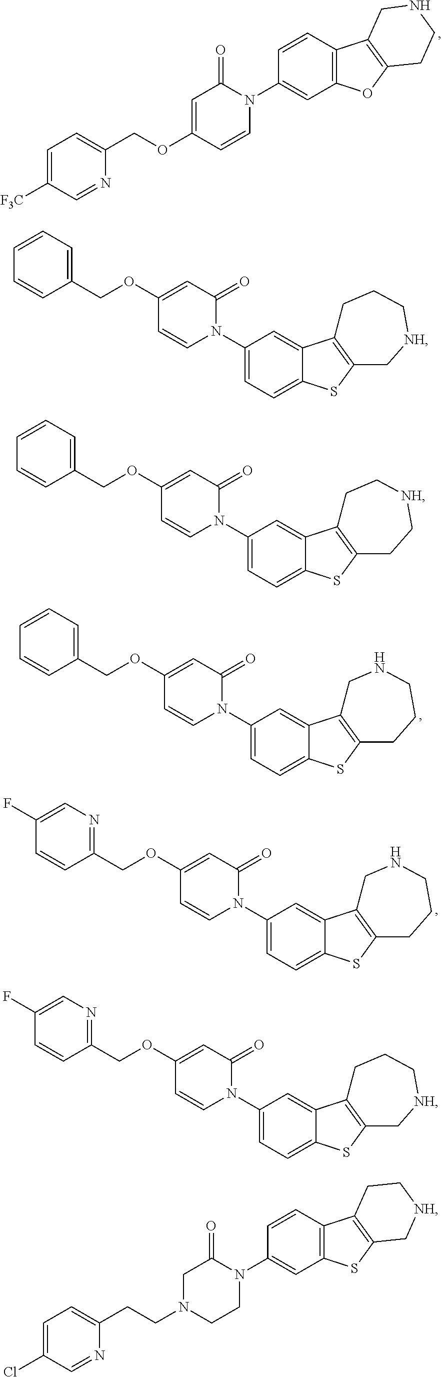

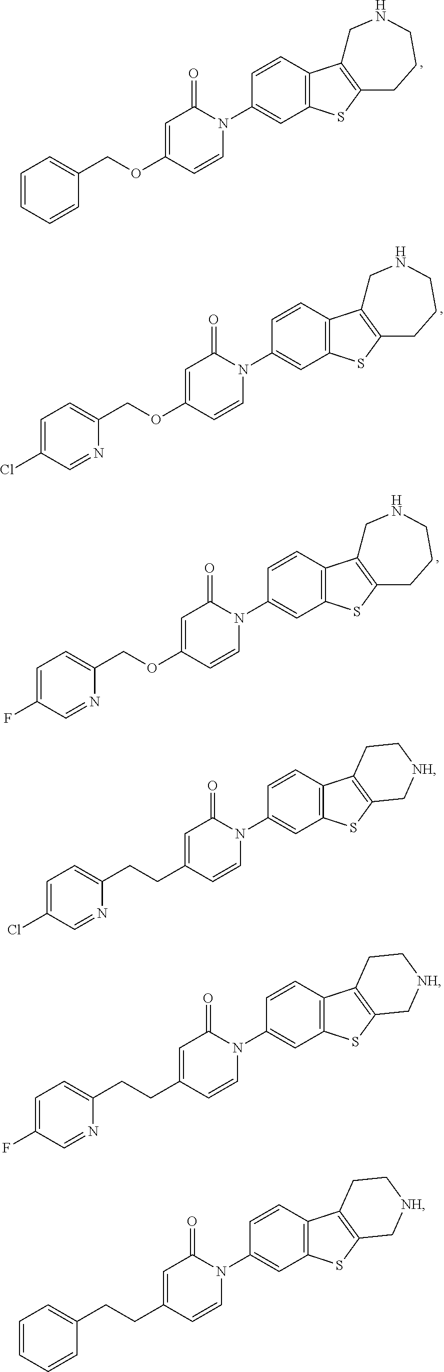 [SMILES: O=c1cc(OCc2ccccc2)ccn1-c1ccc2c3c(sc2c1)CCCNC3 ; O=c1cc(OCc2ccc(Cl)cn2)ccn1-c1ccc2c3c(sc2c1)CCCNC3 ; O=c1cc(OCc2ccc(F)cn2)ccn1-c1ccc2c3c(sc2c1)CCCNC3 ; O=c1cc(CCc2ccc(Cl)cn2)ccn1-c1ccc2c3c(sc2c1)CNCC3 ; O=c1cc(CCc2ccc(F)cn2)ccn1-c1ccc2c3c(sc2c1)CNCC3 ; O=c1cc(CCc2ccccc2)ccn1-c1ccc2c3c(sc2c1)CNCC3]

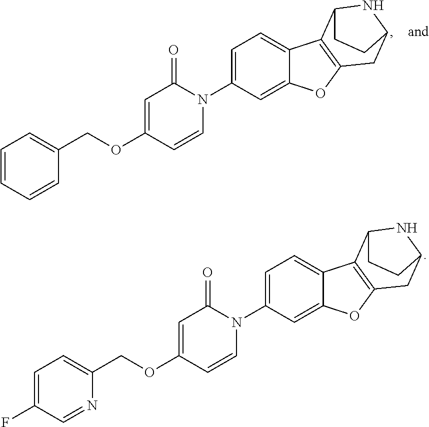 [SMILES: O=c1cc(OCc2ccccc2)ccn1-c1ccc2c3c(oc2c1)CC1CCC3N1 ; O=c1cc(OCc2ccc(F)cn2)ccn1-c1ccc2c3c(oc2c1)CC1CCC3N1]

In accordance with another embodiment, the compound is selected from

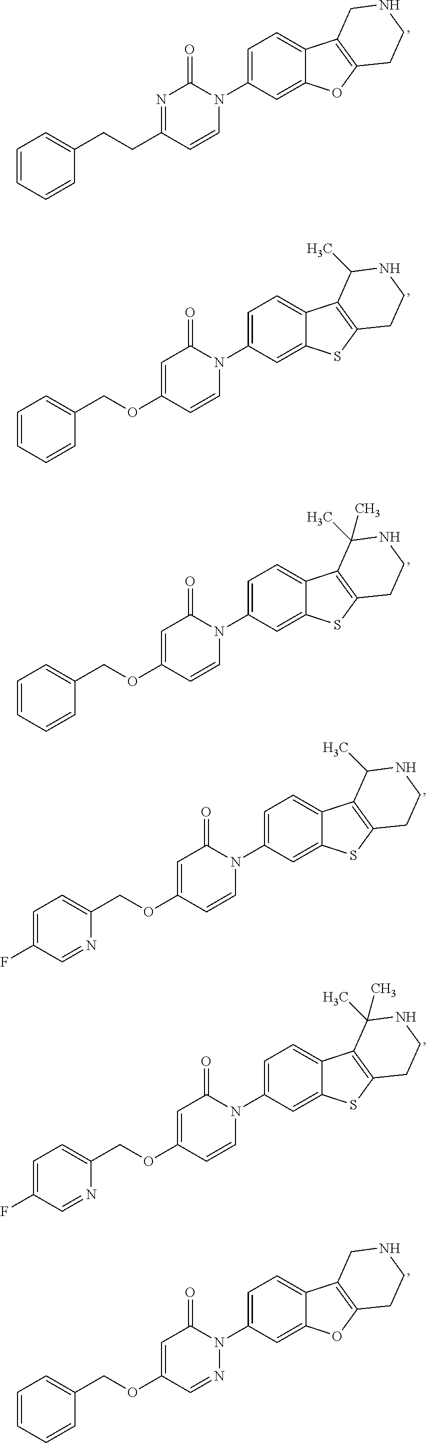 [SMILES: O=c1nc(CCc2ccccc2)ccn1-c1ccc2c3c(oc2c1)CCNC3 ; CC1NCCc2sc3cc(-n4ccc(OCc5ccccc5)cc4=O)ccc3c21 ; CC1(C)NCCc2sc3cc(-n4ccc(OCc5ccccc5)cc4=O)ccc3c21 ; CC1NCCc2sc3cc(-n4ccc(OCc5ccc(F)cn5)cc4=O)ccc3c21 ; CC1(C)NCCc2sc3cc(-n4ccc(OCc5ccc(F)cn5)cc4=O)ccc3c21 ; O=c1cc(OCc2ccccc2)cnn1-c1ccc2c3c(oc2c1)CCNC3]

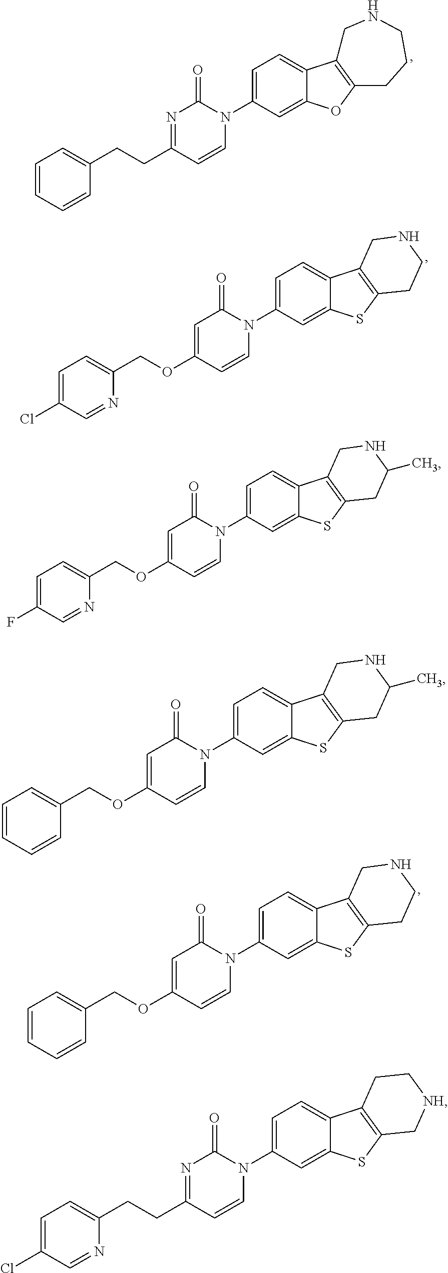 [SMILES: O=c1nc(CCc2ccccc2)ccn1-c1ccc2c3c(oc2c1)CCCNC3 ; O=c1cc(OCc2ccc(Cl)cn2)ccn1-c1ccc2c3c(sc2c1)CCNC3 ; CC1Cc2sc3cc(-n4ccc(OCc5ccc(F)cn5)cc4=O)ccc3c2CN1 ; CC1Cc2sc3cc(-n4ccc(OCc5ccccc5)cc4=O)ccc3c2CN1 ; O=c1cc(OCc2ccccc2)ccn1-c1ccc2c3c(sc2c1)CCNC3 ; O=c1nc(CCc2ccc(Cl)cn2)ccn1-c1ccc2c3c(sc2c1)CNCC3]

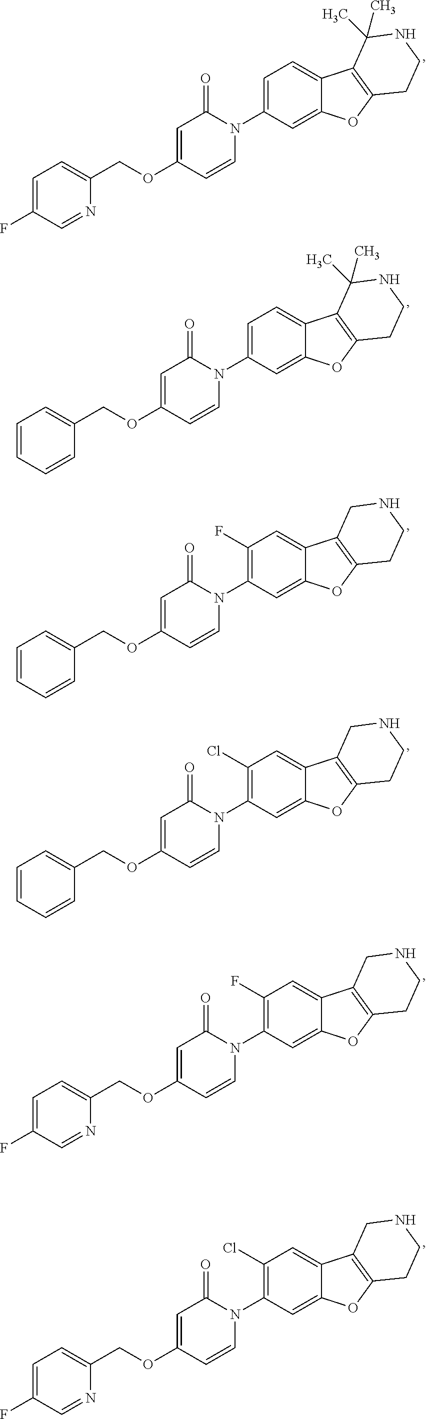 [SMILES: CC1(C)NCCc2oc3cc(-n4ccc(OCc5ccc(F)cn5)cc4=O)ccc3c21 ; CC1(C)NCCc2oc3cc(-n4ccc(OCc5ccccc5)cc4=O)ccc3c21 ; O=c1cc(OCc2ccccc2)ccn1-c1cc2oc3c(c2cc1F)CNCC3 ; O=c1cc(OCc2ccccc2)ccn1-c1cc2oc3c(c2cc1Cl)CNCC3 ; O=c1cc(OCc2ccc(F)cn2)ccn1-c1cc2oc3c(c2cc1F)CNCC3 ; O=c1cc(OCc2ccc(F)cn2)ccn1-c1cc2oc3c(c2cc1Cl)CNCC3]

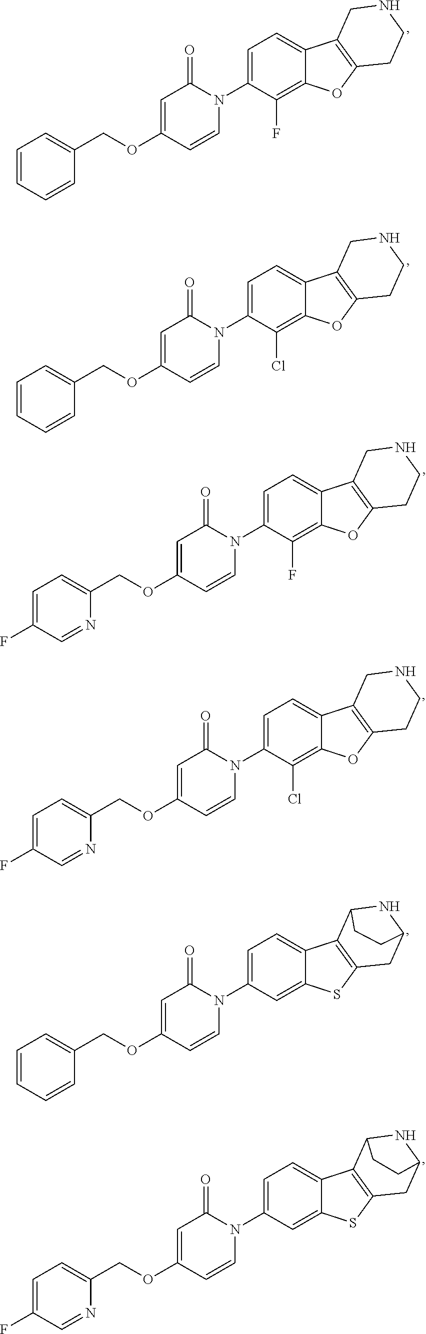 [SMILES: O=c1cc(OCc2ccccc2)ccn1-c1ccc2c3c(oc2c1F)CCNC3 ; O=c1cc(OCc2ccccc2)ccn1-c1ccc2c3c(oc2c1Cl)CCNC3 ; O=c1cc(OCc2ccc(F)cn2)ccn1-c1ccc2c3c(oc2c1F)CCNC3 ; O=c1cc(OCc2ccc(F)cn2)ccn1-c1ccc2c3c(oc2c1Cl)CCNC3 ; O=c1cc(OCc2ccccc2)ccn1-c1ccc2c3c(sc2c1)CC1CCC3N1 ; O=c1cc(OCc2ccc(F)cn2)ccn1-c1ccc2c3c(sc2c1)CC1CCC3N1]

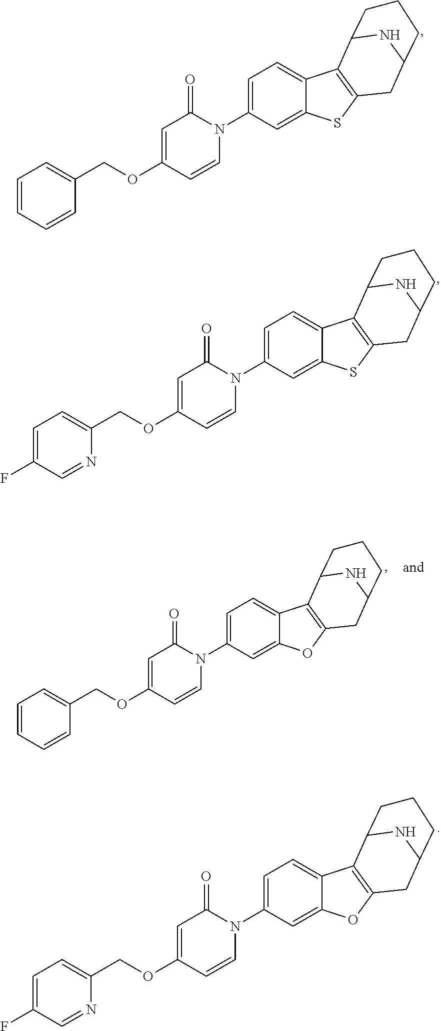 [SMILES: O=c1cc(OCc2ccccc2)ccn1-c1ccc2c3c(sc2c1)CC1CCCC3N1 ; O=c1cc(OCc2ccc(F)cn2)ccn1-c1ccc2c3c(sc2c1)CC1CCCC3N1 ; O=c1cc(OCc2ccccc2)ccn1-c1ccc2c3c(oc2c1)CC1CCCC3N1 ; O=c1cc(OCc2ccc(F)cn2)ccn1-c1ccc2c3c(oc2c1)CC1CCCC3N1]

Tables 1-2, infra, list compounds representative of embodiments of this technology.

One embodiment relates to pharmaceutically acceptable salts, or non-salt forms, of any of the compounds of formula I described herein. In one embodiment, the salt is a HCl salt.

Single enantiomers, any mixture of enantiomers, including racemic mixtures, or diastereomers (both separated and as any mixtures) of the compounds described herein are also included as embodiments of this technology.

The scope of this technology also encompasses active metabolites of the present compounds.

This technology also includes compounds of formula I, wherein one or more of the atoms, e.g., C or H, are replaced by the corresponding radioactive isotopes of that atom (e.g., C replaced by ¹⁴C and H replaced by ³H), or a stable isotope of that atom (e.g., C replaced by ¹³C or H replaced by ²H). Radioisotopes of hydrogen, carbon, phosphorous, fluorine, iodine and chlorine include ³H, ¹⁴C, ³⁵S, ¹⁸F, ³²P, ³³P, ¹²⁵I, and ³⁶Cl, respectively. Compounds that contain those radioisotopes and/or other radioisotopes of other atoms are within the scope of this technology. Radiolabeled compounds described herein and prodrugs thereof can generally be prepared by methods well known to those skilled in the art. Conveniently, such radiolabeled compounds can be prepared by carrying out the procedures disclosed in the Examples and Schemes by substituting a readily available radiolabeled reagent for a non-radiolabeled reagent. Such compounds have a variety of potential uses, e.g., as standards and reagents in determining the ability of a potential pharmaceutical to bind to neurotransmitter proteins. In addition, in the case of stable isotopes, such compounds may have the potential to favorably modify the biological properties, e.g., pharmacological and/or pharmacokinetic properties, of compounds of formula I. The details concerning selection of suitable sites for incorporating radioactive isotopes into the compounds are known to those skilled in the art.

Compounds as described herein are useful as MCH-1 receptor antagonists. It may be found upon examination that compounds that are not presently excluded from the claims are not patentable to the inventors in this application. In that case, the exclusion of species and genera in applicants' claims are to be considered artifacts of patent prosecution and not reflective of the inventors' concept or description of their invention. The invention, in a compound aspect, is all compounds of formula I as described herein, except those that are in the public's possession.

While it may be possible for compounds of formula I to be administered as the raw chemical, it will often be desirable to present them as part of a pharmaceutical composition. Accordingly, another aspect of this technology is a pharmaceutical composition containing a therapeutically effective amount of a compound of formula I, or a pharmaceutically acceptable salt or solvate thereof, and a pharmaceutically acceptable carrier. The carrier must be “acceptable” in the sense of being compatible with the other ingredients of the formulation and not deleterious to the recipient thereof. Furthermore, when reference is made in an independent claim to a compound or a pharmaceutically acceptable salt thereof, it will be understood that claims which depend from that independent claim which refer to such a compound also include pharmaceutically acceptable salts of the compound, even if explicit reference is not made to the salts.

Solid carriers suitable for use in the compositions described herein include one or more substances which may also act as flavoring agents, lubricants, solubilizers, suspending agents, fillers, glidants, compression aides, binders, tablet-disintegrating agents, or encapsulating materials. In powders, the carrier may be a finely divided solid which is in admixture with a finely divided compound of formula I. In tablets, the formula I compound may be mixed with a carrier having the necessary compression properties in suitable proportions and compacted in the shape and size desired. Said powders and tablets may contain up to 99% by weight of the formula I compound. Solid carriers suitable for use in the compositions described herein include calcium phosphate, magnesium stearate, talc, sugars, lactose, dextrin, starch, gelatin, cellulose, methyl cellulose, sodium carboxymethyl cellulose, polyvinylpyrrolidine, low melting waxes, and ion exchange resins.

Any pharmaceutically acceptable liquid carrier suitable for preparing solutions, suspensions, emulsions, syrups and elixirs may be employed in the compositions described herein. Compounds of formula I may be dissolved or suspended in a pharmaceutically acceptable liquid carrier such as water, an organic solvent, or a pharmaceutically acceptable oil or fat, or a mixture thereof. Said liquid composition may contain other suitable pharmaceutical additives such as solubilizers, emulsifiers, buffers, preservatives, sweeteners, flavoring agents, suspending agents, thickening agents, coloring agents, viscosity regulators, stabilizers, osmo-regulators, or the like. Examples of liquid carriers suitable for oral and parenteral administration include water (particularly containing additives as above, e.g., cellulose derivatives, such as sodium carboxymethyl cellulose solution), alcohols (including monohydric alcohols and polyhydric alcohols, e.g., glycols) or their derivatives, or oils (e.g., fractionated coconut oil and arachis oil). For parenteral administration the carrier may also be an oily ester such as ethyl oleate or isopropyl myristate.

In one embodiment, the pharmaceutical composition further comprises one or more other therapeutic adjuncts, e.g., other compounds effective in the treatment of obesity, anxiety, depression, or non-alcoholic fatty liver disease, that are known to persons of skill in the art. Such other therapeutic adjuncts are described below.

Another aspect of this technology relates to a method of treating a disease or condition which is susceptible to treatment with an MCH-1 receptor antagonist. This method involves selecting a patient with a disease or condition which is susceptible to treatment with an MCH-1 receptor antagonist and administering to the patient a therapeutically effective amount of a compound of formula I or a pharmaceutically acceptable salt thereof.

Diseases or conditions which are susceptible to treatment with an MCH-1 receptor antagonist include, but are not limited to, obesity, general anxiety disorders, inflammatory bowel disease, social phobias, vertigo, obsessive-compulsive disorders, panic disorders, post-traumatic stress disorders, Parkinson's Disease Psychosis, schizophrenia, cognitive decline and defects in schizophrenia, Parkinson's Disease, Huntington's Chorea, presenile dementias, Alzheimer's Disease, psychological disorders, depression, substance abuse disorders, dementia associated with neurodegenerative disease, cognition deficits, and epilepsy (see PCT Publication No. WO 2007/010275, which is hereby incorporated by reference in its entirety).

As described above, the compounds described herein are useful as MCH-1 antagonists. As used herein, the term “antagonist” refers to a compound which binds to, and decreases the activity of, a receptor in the presence of an agonist.

As used herein, treatment means any manner in which one or more of the symptoms of a disease or disorder are ameliorated or otherwise beneficially altered. Treatment also encompasses any pharmaceutical use of the compositions herein, such as use for treating diseases or disorders in which MCH-1 receptor activity is implicated.

In another embodiment, the above method further involves administering a therapeutically effective amount of one or more therapeutic adjuncts. Suitable therapeutic adjuncts include, but are not limited to, anti-obesity and/or anorectic agents, anti-anxiety agents, anti-depression agents, and anti-non-alcoholic fatty liver disease agents.

Suitable anti-obesity and/or anorectic adjuncts include, but are not limited to, phenylpropanolamine, ephedrine, pseudoephedrine, phentermine, a cholecystokinin-A (hereinafter referred to as CCK-A) agonist, a monoamine reuptake inhibitor (such as sibutramine), a sympathomimetic agent, a serotonergic agent (such as dexfenfluramine or fenfluramine), a dopamine agonist (such as bromocriptine), a melanocyte-stimulating hormone receptor agonist or mimetic, a melanocyte-stimulating hormone analog, a cannabinoid receptor antagonist or inverse agonist, a melanin concentrating hormone receptor antagonist, a serotonin 5-HT₆ receptor antagonist, a serotonin 5-HT_{2C} receptor agonist, the OB protein (hereinafter referred to as “leptin”), a leptin analog, a leptin receptor agonist, the amylin peptide, an amylin analog, an amylin receptor agonist, a neuropeptide Y receptor modulator, a galanin antagonist, or a GI lipase inhibitor or decreaser (such as orlistat). Other anorectic agents include bombesin agonists, dehydroepiandrosterone or analogs thereof, glucocorticoid receptor agonists and antagonists, orexin receptor antagonists, urocortin binding protein antagonists, agonists of the glucagon-like peptide-1 receptor such as Exendin and ciliary neurotrophic factors such as Axokine.

Suitable anti-anxiety adjuncts include, but are not limited to, an allosteric modulator of the GABA_{A} receptor (such as diazepam, lorazepam, or alprazolam), a serotonin 5-HT_{1A} receptor partial agonist (such as buspirone), a selective serotonin reuptake inhibitor (SSRI, such as citalopram, escitalopram, fluoxetine, paroxetine, or sertraline), a serotonin-norepinephrine reuptake inhibitor (SNRI, such as duloxetine or venlafaxine), a monoamine neurotransmitter reuptake inhibitor of the tricyclic antidepressant (TCA) class (such as amitriptyline, desipramine, or imipramine), a combined serotonin reuptake inhibitor and 5-HT_{2C} antagonist (such as trazodone), and an H₁ receptor antagonist (such as hydroxyzine).

Suitable anti-depression adjuncts include, but are not limited to, a serotonin 5-HT_{1A} receptor partial agonist (such as buspirone), a selective serotonin reuptake inhibitor (SSRI, such as citalopram, escitalopram, fluoxetine, paroxetine, or sertraline), a serotonin-norepinephrine reuptake inhibitor (SNRI, such as duloxetine or venlafaxine), a monoamine neurotransmitter reuptake inhibitor of the tricyclic antidepressant (TCA) class (such as amitriptyline, desipramine, or imipramine), a combined serotonin reuptake inhibitor and 5-HT_{2C} antagonist (such as trazodone), a noradrenergic and specific serotonergic antidepressant (NaSSA, such as mianserin or mirtazapine), a norepinephrine reuptake inhibitor (NRI, such as atomoxetine or Mazindol), a norepinephrine-dopamine reuptake inhibitor (NDRI, such as bupropion), and a monoamine oxidase inhibitor (MAOI, such as isocarboxazid or moclobemide).

Suitable anti-non-alcoholic fatty liver disease adjuncts include, but are not limited to, an AMP-activated protein kinase (AMPK) agonist (such as metformin), a peroxisome proliferator-activated receptor (PPAR) gamma activator (such as rosiglitazone, pioglitazone, or troglitazone), a HMG-CoA reductase inhibitor (such as atorvastatin or simvastatin), and a PDE4 inhibitor (such as pentoxifylline).

In one embodiment, the patient is a mammal. The term “mammal” is used in its dictionary sense. The term “mammal” includes, for example, mice, hamsters, rats, cows, sheep, pigs, goats, and horses, monkeys, dogs (e.g., Canis familiaris), cats, rabbits, guinea pigs, and primates, including humans.

This technology also relates to a method of treating obesity in a subject in need of weight loss. This method involves selecting a patient in need of weight loss and administering to the patient a therapeutically effective amount of a compound of formula I or a pharmaceutically acceptable salt thereof.

This method further involves administering an anti-obesity adjunct, as described above.

Yet another aspect relates to a method of treating obesity in a subject who has experienced weight loss. This method involves selecting a patient who has experienced weight loss and administering to the patient a therapeutically effective amount of a compound of formula I or a pharmaceutically acceptable salt thereof.

A further aspect relates to a method of treating anxiety. This method involves selecting a patient with anxiety and administering to the patient a therapeutically effective amount of a compound of formula I or a pharmaceutically acceptable salt thereof.

This method further involves administering an anti-anxiety adjunct, as described above.

This technology also relates to a method of treating depression. This method involves selecting a patient with depression and administering to the patient a therapeutically effective amount of a compound of formula I or a pharmaceutically acceptable salt thereof.

This method further involves administering an anti-depression adjunct, as described above.

Another aspect relates to a method of treating non-alcoholic fatty liver disease. This method involves selecting a patient who has non-alcoholic fatty liver disease and administering to the patient a therapeutically effective amount of a compound of formula I or a pharmaceutically acceptable salt thereof.

This method further involves administering an anti-non-alcoholic fatty liver disease adjunct, as described above.

This technology also relates to a method of treating inflammatory bowel disease. This method involves selecting a patient with inflammatory bowel disease and administering to the patient a therapeutically effective amount of a compound of formula I of a pharmaceutically acceptable salt thereof.

It is appreciated that certain features described herein, which are, for clarity, described in the context of separate embodiments, may also be provided in combination in a single embodiment. Conversely, various features described herein which are, for brevity, described in the context of a single embodiment, may also be provided separately or in any suitable subcombination.

This technology also relates to a process for preparation of a product compound of formula I:

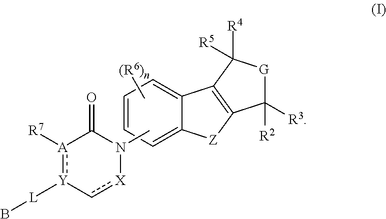

This process involves treating a first intermediate of formula II:

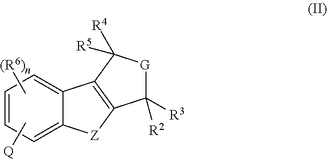

wherein Q is a halogen, under conditions effective to form the product compound of formula I, wherein R¹-R¹⁸, G, X, Y, Z, L, A, and B are as defined above.

In one embodiment, treating involves reacting the first intermediate with a second intermediate having the structure:

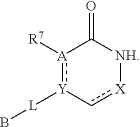

In another embodiment, the process further comprises treating a third intermediate of formula (IV):

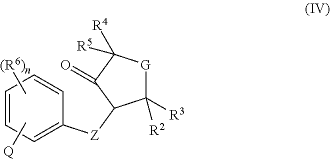

under conditions effective to form the first intermediate compound.

In a further embodiment, the process further comprises reacting

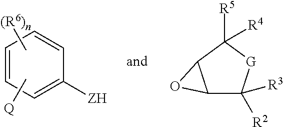

under conditions effective to form a fourth intermediate of formula (V):

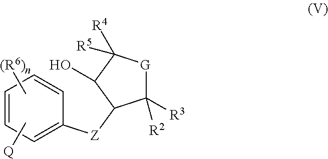

and treating the fourth intermediate compound under conditions effective to form the third intermediate compound.

Compounds useful according to this technology may be prepared by the application or adaptation of known methods, by which is meant methods used heretofore or described in the literature, for example, those described by Larock, Comprehensive Organic Transformations, Wiley-VCH publishers, New York (1989), which is hereby incorporated by reference in its entirety.

A compound of formula I including a group containing one or more nitrogen ring atoms, may be converted to the corresponding compound wherein one or more nitrogen ring atom of the group is oxidized to an N-oxide, in one embodiment by reacting with a peracid, for example peracetic acid in acetic acid or m-chloroperoxybenzoic acid in an inert solvent such as dichloromethane, at a temperature from about room temperature to reflux, or at elevated temperature.

In the reactions described hereinafter and in the claims, it may be necessary to protect reactive functional groups, for example hydroxy, amino, imino, thio, or carboxy groups, where these are desired in the final product, to avoid their unwanted participation in the reactions. Conventional protecting groups may be used in accordance with standard practice and as described above.

The novel MCH-1 antagonists of formula I described herein can be prepared by the methods illustrated in the general reaction schemes as, for example, described below, or by modifications thereof, using readily available starting materials, reagents, and conventional synthesis procedures. In these reactions, it is also possible to make use of variants that are known in the art but are not mentioned here. Although the syntheses depicted herein may result in the preparation of enantiomers having a particular stereochemistry, included within the scope described herein are compounds of formula I in any stereoisomeric form, and preparation of compounds of formula I in stereoisomeric forms other than those depicted herein would be obvious to one of ordinary skill in the chemical arts based on the procedures presented herein.

### Synthetic Methods

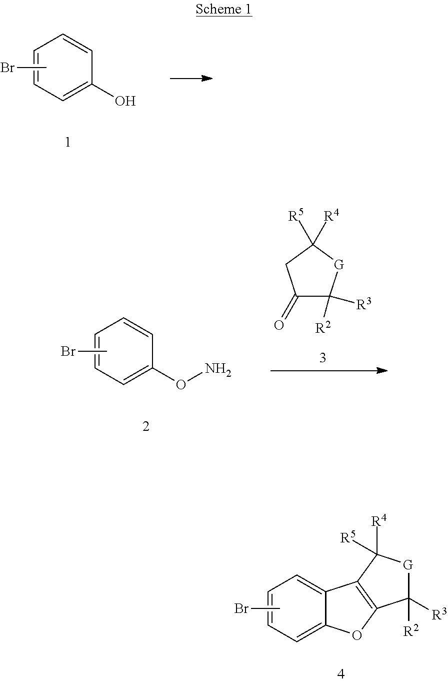

Compounds of formula 2 can be prepared through the treatment of compounds of formula 1 with hydroxylamine-O-sulfonic acid under heated basic conditions. Compounds of formula 2 (or a salt thereof) can be treated with compounds of formula 3 (wherein G is —NR⁸—CR⁹R¹⁰—, —CR⁹R¹⁰—NR⁸—, —NR⁸—CR⁹R¹⁰—CR¹¹R¹²—, —CR⁹R¹⁰—NR⁸—CR¹¹R¹²—, or —CR⁹R¹⁰—CR¹¹R¹²—NR⁸—; R²-R⁵ and R⁹-R¹² are each, independently, H or alkyl; R⁸ is H, alkyl, or a protecting group such as tert-butoxycarbonyl or benzyloxycarbonyl; and, in the case where G is —NR⁸—CR⁹R¹⁰—, R⁴ and R⁹ can be combined to form a C₁-C₄ alkyl bridge) under heated acidic conditions to give compounds of formula 4. In the case where R⁸ is H, optional alkylation or protection of compound 4 can provide compounds of formula 4 wherein R⁸ is alkyl or a protecting group such as tert-butoxycarbonyl or benzyloxycarbonyl.

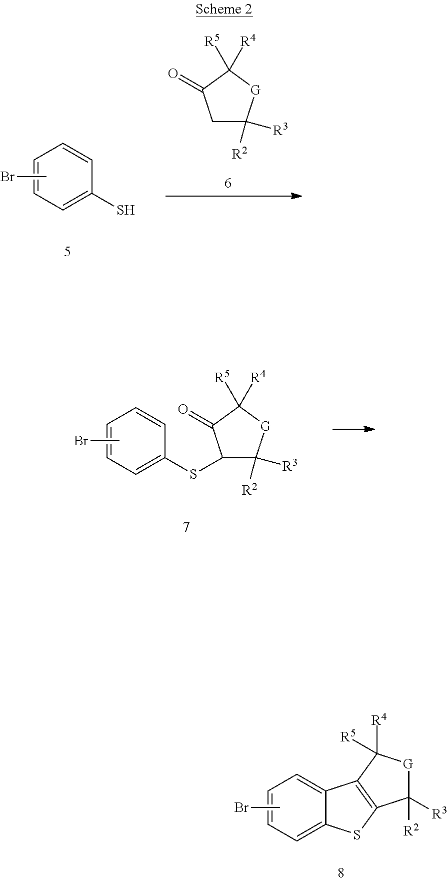

Compounds of formula 6 (wherein G is —NR⁸—CR⁹R¹⁰—, —CR⁹R¹⁰—NR⁸—, —NR⁸—CR⁹R¹⁰—CR¹¹R¹²—, —CR⁹R¹⁰—NR⁸—CR¹¹R¹²—, or —CR⁹R¹⁰—CR¹¹R¹²—NR⁸—; R²-R⁵ and R⁹-R¹² are each, independently, H or alkyl; R⁸ is a protecting group such as tert-butoxycarbonyl or benzyloxycarbonyl; and, in the case where G is —NR⁸—CR⁹R¹⁰—, R⁴ and R⁹ can be combined to form a C₁-C₄ alkyl bridge) can be treated with a halogenating agent such as N-chlorosuccinimide in the presence of thiol 5 to give compounds of formula 7. Compounds of formula 7 can be treated under heated acidic conditions to provide compounds of formula 8 (wherein G is —NR⁸—CR⁹R¹⁰—, —CR⁹R¹⁰—NR⁸—, —NR⁸—CR⁹R¹⁰—CR¹¹R¹²—, —CR⁹R¹⁰—NR⁸—CR¹¹R¹²—, or —CR⁹R¹⁰—CR¹¹R¹²—NR⁸—; R²-R⁵ and R⁹-R¹² are each, independently, H or alkyl; R⁸ is H or a protecting group such as tert-butoxycarbonyl or benzyloxycarbonyl; and, in the case where G is —NR⁸—CR⁹R¹⁰—, R⁴ and R⁹ can be combined to form a C₁-C₄ alkyl bridge). In the case where R⁸ is H, optional alkylation or protection of compound 8 can provide compounds of formula 8 wherein R⁸ is alkyl or a protecting group such as tert-butoxycarbonyl or benzyloxycarbonyl.

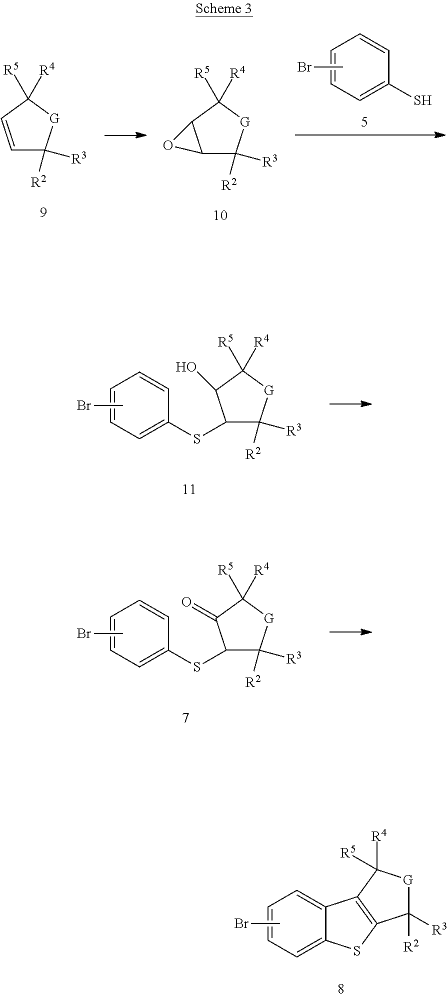

Alternatively, compounds of formula 9 (wherein G is —NR⁸—CR⁹R¹⁰—, —CR⁹R¹⁰—NR⁸—, —NR⁸—CR⁹R¹⁰—CR¹¹R¹²—, —CR⁹R¹⁰—NR⁸—CR¹¹R¹²—, or —CR⁹R¹⁰—CR¹¹R¹²—NR⁸—; R²-R⁵ and R⁹-R² are each, independently, H or alkyl; R⁸ is a protecting group such as tert-butoxycarbonyl or benzyloxycarbonyl; and, in the case where G is —NR⁸—CR⁹R¹⁰—, R⁴ and R⁹ can be combined to form a C₁-C₄ alkyl bridge) can be treated with an oxidizing agent such as m-chloroperoxybenzoic acid to provide compounds of formula 10. Treatment of compounds of formula 10 with thiol 5 under heated basic conditions or Lewis acidic conditions can provide compounds of formula 11. Compounds of formula 11 can be treated with an oxidizing agent such as Dess-Martin periodinane to provide compounds of formula 7. Treatment of compounds of formula 7 under heated acidic conditions can provide compounds of formula 8 (wherein G is —NR⁸—CR⁹R¹⁰—, —CR⁹R¹⁰—NR⁸—, —NR⁸—CR⁹R¹⁰—CR¹¹R¹²—, —CR⁹R¹⁰—NR⁸—CR¹¹R¹²—, or —CR⁹R¹⁰—CR¹¹R¹²—NR⁸—; R²-R⁵ and R⁹-R¹² are each, independently, H or alkyl; R⁸ is H or a protecting group such as tert-butoxycarbonyl or benzyloxycarbonyl; and, in the case where G is —NR⁸—CR⁹R¹⁰—, R⁴ and R⁹ can be combined to form a C₁-C₄ alkyl bridge). In the case where R⁸ is H, optional alkylation or protection of compound 8 can provide compounds of formula 8 wherein R⁸ is alkyl or a protecting group such as tert-butoxycarbonyl or benzyloxycarbonyl.

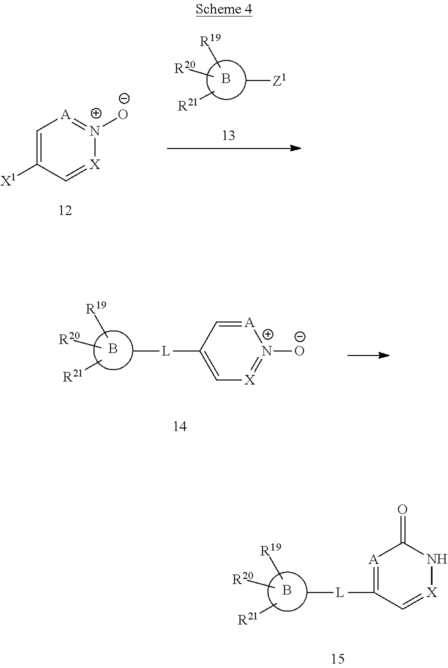

Compounds of formula 15 (wherein B is aryl, heteroaryl, heterocyclyl, or cycloalkyl; R¹⁹, R²⁰, and R²¹ are each independently selected from H, alkoxy, S-alkyl, alkyl, halo, —CF₃, and —CN; A is CH; X is CH; and L is —CH₂—O— or a bond) can be prepared by treating compounds of formula 12 (wherein X¹ is chlorine, bromine or iodine; A is CH; and X is CH) with compounds of formula 13 (wherein B is aryl, heteroaryl, heterocyclyl, or cycloalkyl; R¹⁹, R²⁰, and R²¹ are each independently selected from H, —O-alkyl, alkyl, halo, —CF₃, and —CN; Z¹ is B(OH)₂, B(OR²²)₂, SnR²²₃ or the like and R²² is alkyl), a catalyst such as palladium(0), and a base such as potassium carbonate to give compounds of formula 14, wherein L is a direct bond. Alternatively, in the case where Z¹ is —CH₂—OH and B is aryl, heteroaryl, heterocyclyl, or cycloalkyl, compounds of formula 13 can be treated with a base such as sodium hydride and compounds of formula 12 under heated conditions to give compounds of formula 14, wherein L is —CH₂—O—. In turn, compounds of formula 14 can be treated with acetic anhydride under heated conditions followed by methanol and water or methanol and sodium hydroxide under ambient to heated conditions to provide compounds of formula 15, wherein L is —CH₂O— or a direct bond.

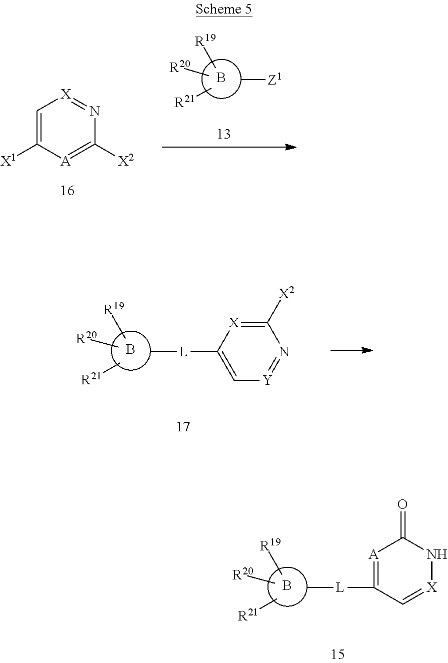

Alternatively, compounds of formula 15 (wherein B is aryl, heteroaryl, heterocyclyl, or cycloalkyl; R¹⁹, R²⁰, and R²¹ are each independently selected from H, alkoxy, —S-alkyl, alkyl, halo, —CF₃, and —CN; A is CH or N; X is CH; and L is —CH₂—CH₂—, or a bond) can be prepared by treating compounds of formula 16 (wherein X¹ is chlorine, bromine or iodine; X² is —O—CH₃ or chlorine; A is CH or N; and X is CH) with compounds of formula 13 (wherein Z¹ is —CH═CH—B(OR²²)₂, B(OH)₂, B(OR²²)₂, SnR²²₃ or the like and R²² is alkyl), a catalyst such as palladium(0), and a base such as potassium carbonate to give compounds of formula 17, wherein L is —CH═CH— or a direct bond, in accordance with Z¹. In the case where L is —CH═CH—, compounds of formula 17 can be treated with palladium on carbon under an atmosphere of hydrogen to give compounds of formula 17, wherein L is —CH₂CH₂—. Alternatively, in the case where Z¹ is —CH₂—OH, compounds of formula 16 can be treated with compounds of formula 13, a catalyst such as copper iodide, a ligand such as 3,4,7,8-tetramethylphenanthroline and a base such as cesium carbonate under heated conditions to give compounds of formula 17, wherein L is —CH₂—O—. In turn, when L is —CH₂—CH₂—, —CH₂—O— or a direct bond, compounds of formula 17 can be heated under acid conditions to provide compounds of formula 15, wherein L is —CH₂—CH₂—, —CH₂—O— or a direct bond, respectively.

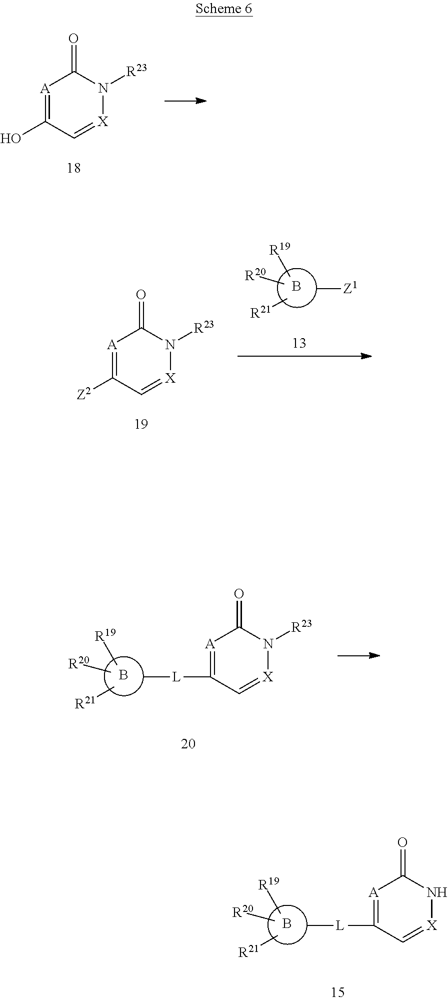

Alternatively, compounds of formula 15 (wherein B is aryl, heteroaryl, heterocyclyl, or cycloalkyl; R¹⁹, R²⁰, and R²¹ are each independently selected from H, alkoxy, S-alkyl, alkyl, halo, —CF₃, and —CN; A is CH; X is N; and L is —CH₂—O— or a bond) can be prepared from compounds of formula 18 (wherein A is CH; X is N; and R²³ is a protecting group such as tetrahydropyran-2-yl). The hydroxyl group on compound 18 can be converted to an appropriate activating group to give compounds of formula 19. In the case where Z² is triflate, compounds of formula 18 can be treated with trifluoromethylsulfonic anhydride or N-phenyl trifluoromethanesulfonamide and a base such as triethylamine, pyridine or lithium bis(trimethylsilyl)amide under cooled conditions to give compounds of formula 19. Treatment of compounds of formula 19 with compounds of formula 13 (wherein B is aryl, heteroaryl, heterocyclyl, or cycloalkyl; R¹⁹, R²⁰, and R²¹ are each independently selected from H, alkoxy, S-alkyl, alkyl, halo, —CF₃, and —CN; Z¹ is B(OH)₂, B(OR²²)₂, SnR²²₃ or the like, and R²² is alkyl), a catalyst such as palladium(0), and a base such as potassium carbonate under heated conditions can provide compounds of formula 20, wherein L is a direct bond. Alternatively, in the case where Z¹ is —CH₂—Br, compounds of formula 13 can be treated with compounds of formula 18 and a base such as potassium carbonate to give compounds of formula 20, wherein L is —CH₂—O—. Removal of the protecting group R²³ on compound 20 can provide compounds of formula 15, wherein L is —CH₂—O— or a bond.

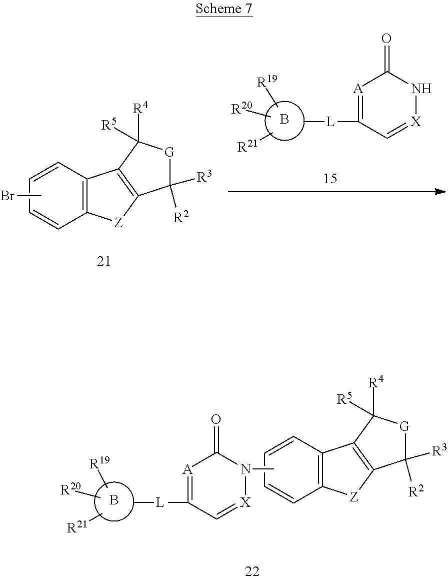

Compounds of formula 22 (wherein B is aryl, heteroaryl, heterocyclyl, or cycloalkyl; R¹⁹, R²⁰, and R²¹ are each independently selected from H, alkoxy, —S-alkyl, alkyl, halo, —CF₃, and —CN; L is —CH₂—CH₂—, —CH₂—O— or a bond; A is CH or N; X is CH or N; Z is O or S; G is —NR⁸—CR⁹R¹⁰—, —CR⁹R¹⁰—NR⁸—, —NR⁸—CR⁹R¹⁰—CR¹¹R¹²—, —CR⁹R¹⁰—NR⁸—CR¹¹R¹²—, or —CR⁹R¹⁰—CR¹¹R¹²—NR⁸—; R²-R⁵ and R⁹-R¹² are each, independently, H or alkyl; R⁸ is H, alkyl, or a protecting group such as tert-butoxycarbonyl or benzyloxycarbonyl; and, in the case where G is —NR⁸—CR⁹R¹⁰—, R⁴ and R⁹ can be combined to form a C₁-C₄ alkyl bridge) can be prepared by treating compounds of formula 21 (wherein Z is O or S; G is —NR⁸—CR⁹R¹⁰—, —CR⁹R¹⁰—NR⁸—, —NR⁸—CR⁹R¹⁰—CR¹¹R¹²—, —CR⁹R¹⁰—NR⁸—CR¹¹R¹²—, or —CR⁹R¹⁰—CR¹¹R¹²—NR⁸—; R²-R⁵ and R⁹-R¹² are each, independently, H or alkyl; R⁸ is H, alkyl, or a protecting group such as tert-butoxycarbonyl or benzyloxycarbonyl; and, in the case where G is —NR⁸—CR⁹R¹⁰—, R⁴ and R⁹ can be combined to form a C₁-C₄ alkyl bridge) under heated conditions with a catalyst such as copper iodide, a ligand such as trans-1,2-diaminocyclohexane, trans-N,N′-dimethylcyclohexane-1,2-diamine, or 8-hydroxyquinoline, a base such as potassium carbonate, cesium carbonate or potassium phosphate and compounds of formula 15 (wherein B is aryl, heteroaryl, heterocyclyl, or cycloalkyl; R¹⁹, R²⁰, and R²¹ are each independently selected from H, alkoxy, —S-alkyl, alkyl, halo, —CF₃, and —CN; L is —CH₂—CH₂—, —CH₂—O— or a bond; A is CH or N; and X is CH or N). In the case where R⁸ is a protecting group, the protecting group can be removed to give compounds of formula 22 wherein R⁸ is H. In the case where R⁸ is H, reductive amination or alkylation can provide compounds of formula 22, wherein R⁸ is an alkyl group.

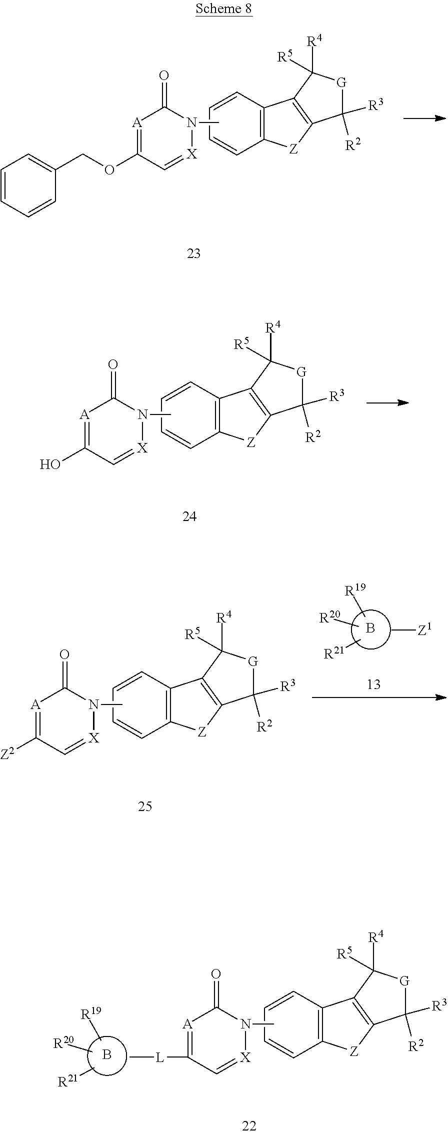

Compounds of formula 23 (wherein A is CH or N; X is CH or N; Z is O or S; G is —NR⁸—CR⁹R¹⁰—, —CR⁹R¹⁰—NR⁸—, —NR⁸—CR⁹R¹⁰—CR¹¹R¹²—, —CR⁹R¹⁰—NR⁸—CR¹¹R¹²—, or —CR⁹R¹⁰—CR¹¹R¹²—NR⁸—; R²-R⁵ and R⁹-R¹² are each, independently, H or alkyl; R⁸ is H, alkyl, or a protecting group such as tert-butoxycarbonyl or benzyloxycarbonyl; and, in the case where G is —NR⁸—CR⁹R¹⁰—, R⁴ and R⁹ can be combined to form a C₁-C₄ alkyl bridge) can be treated with hydrogen and a catalyst such as palladium on carbon to provide compounds of formula 24. The hydroxyl group on compounds of formula 24 can be converted to an appropriate activating group to give compounds of formula 25. In the case where Z² is triflate, compounds of formula 24 can be treated with trifluoromethylsulfonic anhydride or N-phenyl trifluoromethanesulfonamide and a base such as pyridine or lithium bis(trimethylsilyl)amide under cooled conditions to give compounds of formula 25. Treatment of compounds of formula 25 with compounds of formula 13 (wherein B is aryl, heteroaryl, heterocyclyl, or cycloalkyl; R¹⁹, R²⁰, R²¹ are each independently selected from H, alkoxy, —S-alkyl, alkyl, halo, —CF₃, and —CN; Z¹ is —CH═CH—B(OR²²)₂, B(OH)₂, B(OR²²)₂, SnR²²₃ or the like and R²² is alkyl), a catalyst such as palladium(0), and a base such as potassium carbonate under heated conditions can provide compounds of formula 22, wherein L is —CH═CH— or a direct bond. In the case where L is —CH═CH—, compounds of formula 22 can be treated with palladium on carbon under an atmosphere of hydrogen to give compounds of formula 22, where L is —CH₂CH₂—.

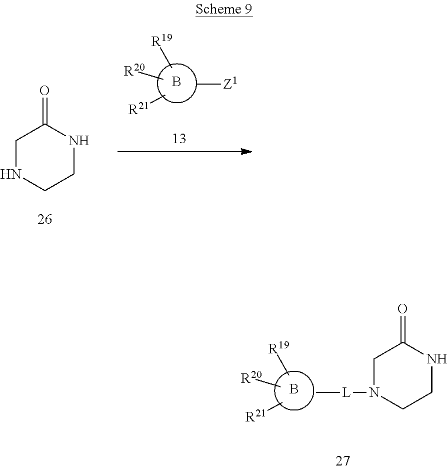

Compounds of formula 27 (wherein B is aryl, heteroaryl, heterocyclyl, or cycloalkyl; R¹⁹, R²⁰, and R²¹ are each independently selected from H, alkoxy, —S-alkyl, alkyl, halo, —CF₃, and —CN; and L is —CH₂—CH₂—) can be prepared by treating piperazin-2-one 26 with compounds of formula 13 (wherein Z¹ is —CH₂—CH₂—X¹; and X¹ is a leaving group such as chlorine, bromine, iodine or the like) and a base such as di-isopropylamine to give compounds of formula 27, wherein L is —CH₂—CH₂—.

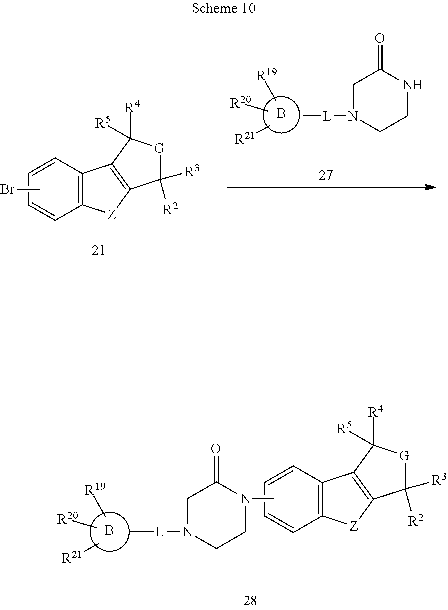

Compounds of formula 28 (wherein B is aryl, heteroaryl, heterocyclyl, or cycloalkyl; R¹⁹, R²⁰, and R²¹ are each independently selected from H, alkoxy, —S-alkyl, alkyl, halo, —CF₃, and —CN; L is —CH₂—CH₂—; Z is O or S; G is —NR⁸—CR⁹R¹⁰—, —CR⁹R¹⁰—NR⁸—, —NR⁸—CR⁹R¹⁰—CR¹¹R¹²—, —CR⁹R¹⁰—NR⁸—CR¹¹R¹²—, or —CR⁹R¹⁰—CR¹¹R¹²—NR⁸—; R²-R⁵ and R⁹-R¹² are each, independently, H or alkyl; R⁸ is H, alkyl, or a protecting group such as tert-butoxycarbonyl or benzyloxycarbonyl; and, in the case where G is —NR⁸—CR⁹R¹⁰—, R⁴ and R⁹ can be combined to form a C₁-C₄ alkyl bridge) can be prepared by treating compounds of formula 21 (wherein Z is O or S; G is —NR⁸—CR⁹R¹⁰—, —CR⁹R¹⁰—NR⁸—, —NR⁸—CR⁹R¹⁰—CR¹¹R¹²—, —CR⁹R¹⁰—NR⁸—CR¹¹R¹²—, or —CR⁹R¹⁰—CR¹¹R¹²—NR⁸—; R²-R⁵ and R⁹-R¹² are each, independently, H or alkyl; R⁸ is H, alkyl, or a protecting group such as tert-butoxycarbonyl or benzyloxycarbonyl; and, in the case where G is —NR⁸—CR⁹R¹⁰—, R⁴ and R⁹ can be combined to form a C₁-C₄ alkyl bridge) under heated conditions with a catalyst such as copper iodide, a ligand such as trans-1,2-bis(methylamino)cyclohexane or 8-hydroxyquinoline, a base such as potassium carbonate, cesium carbonate or potassium phosphate and compounds of formula 27 (wherein B is aryl, heteroaryl, heterocyclyl, or cycloalkyl; R¹⁹, R²⁰, and R²¹ are each independently selected from H, alkoxy, —S-alkyl, alkyl, halo, —CF₃, and —CN; and L is —CH₂—CH₂—). In the case where R⁸ is a protecting group, the protecting group can be removed to give compounds of formula 28 wherein R⁸ is H. In the case where R⁸ is H, reductive amination or alkylation can provide compounds of formula 28, wherein R⁸ is an alkyl group. Additionally, in the case where R⁸ is a protecting group, the protecting group can be removed to give compounds of formula 28 wherein R⁸ is H.

This technology provides compositions containing the compounds described herein, including, in particular, pharmaceutical compositions comprising therapeutically effective amounts of the compounds and pharmaceutically acceptable carriers.

It is a further object of this technology to provide kits having a plurality of active ingredients (with or without carrier) which, together, may be effectively utilized for carrying out the novel combination therapies described herein.

It is another object of this technology to provide a novel pharmaceutical composition which is effective, in and of itself, for utilization in a beneficial combination therapy because it includes a plurality of active ingredients which may be utilized as described herein.

This technology also provides kits or single packages combining one or more active ingredients useful in treating the disease. A kit may provide (alone or in combination with a pharmaceutically acceptable diluent or carrier) the compounds of formula I and an additional active ingredient (alone or in combination with diluent or carrier), as described above.

The products described herein may be presented in forms permitting administration by the most suitable route and this technology also relates to pharmaceutical compositions containing at least one product as described herein which are suitable for use in human or veterinary medicine. These compositions may be prepared according to the customary methods, using one or more pharmaceutically acceptable adjuvants or excipients. The adjuvants comprise, inter alia, diluents, sterile aqueous media, and the various non-toxic organic solvents. The compositions may be presented in the form of tablets, pills, granules, powders, aqueous solutions or suspensions, injectable solutions, elixirs or syrups, and can contain one or more agents chosen from the group comprising sweeteners, flavorings, colorings, or stabilizers in order to obtain pharmaceutically acceptable preparations.

The formulations of compounds of formula I include those suitable for oral, parenteral (including subcutaneous, intradermal, intramuscular, intraperitoneal, intravenous, and intraarticular), rectal, colonic, and topical (including dermal, buccal, nasal, sublingual, and intraocular) administration. The most suitable route may depend upon the condition and disorder of the recipient. The formulations may conveniently be presented in unit dosage form and may be prepared by any of the methods well known in the art of pharmacy. Such methods include the step of bringing into association a compound of formula I or a pharmaceutically acceptable salt or solvate thereof (“active ingredient”) with the carrier, which constitutes one or more accessory ingredients. In general, the formulations are prepared by uniformly and intimately bringing into association the active ingredient with liquid carriers or finely divided solid carriers or both and then, if necessary, shaping the product into the desired formulation.

Formulations suitable for oral administration may be presented as discrete units such as capsules, cachets, or tablets each containing a predetermined amount of the active ingredient; as a powder or granules; as a solution or a suspension in an aqueous liquid or a non-aqueous liquid; or as an oil-in-water liquid emulsion or a water-in-oil liquid emulsion. The active ingredient may also be presented as a bolus, electuary, or paste.

A tablet may be made by compression or molding, optionally with one or more accessory ingredients. Compressed tablets may be prepared by compressing in a suitable machine the active ingredient in a free-flowing form such as a powder or granules, optionally mixed with a binder, lubricant, inert diluent, lubricating, surface active, or dispersing agent. Molded tablets may be made by molding in a suitable machine a mixture of the powdered compound moistened with an inert liquid diluent. The tablets may optionally be coated or scored and may be formulated so as to provide sustained, delayed, or controlled release of the active ingredient therein.

The pharmaceutical compositions may include a “pharmaceutically acceptable inert carrier”, and this expression is intended to include one or more inert excipients, which include starches, polyols, granulating agents, microcrystalline cellulose, diluents, lubricants, binders, disintegrating agents, and the like. If desired, tablet dosages of the disclosed compositions may be coated by standard aqueous or nonaqueous techniques, “Pharmaceutically acceptable carrier” also encompasses controlled release means.

Pharmaceutical compositions may also optionally include other therapeutic ingredients, anti-caking agents, preservatives, sweetening agents, colorants, flavors, desiccants, plasticizers, dyes, and the like. Any such optional ingredient must be compatible with the compound of formula I to insure the stability of the formulation. The composition may contain other additives as needed, including for example lactose, glucose, fructose, galactose, trehalose, sucrose, maltose, raffinose, maltitol, melezitose, stachyose, lactitol, palatinite, starch, xylitol, mannitol, myoinositol, and the like, and hydrates thereof, and amino acids, for example alanine, glycine and betaine, and peptides and proteins, for example albumen.

Examples of excipients for use as the pharmaceutically acceptable carriers and the pharmaceutically acceptable inert carriers and the aforementioned additional ingredients include, but are not limited to binders, fillers, disintegrants, lubricants, anti-microbial agents, and coating agents.

The dose range for adult humans is generally from 0.001 mg to 10 g/day orally. Tablets or other forms of presentation provided in discrete units may conveniently contain an amount of compound of formula I which is effective at such dosage or as a multiple of the same, for instance, units containing 5 mg to 500 mg, usually around 10 mg to 200 mg. The precise amount of compound administered to a patient will be the responsibility of the attendant physician. It will be understood, however, that the specific dose level for any particular patient will depend upon a variety of factors including the activity of the specific compound employed, the age, body weight, general health, sex, diet time of administration, route of administration, rate of excretion, drug combination and the severity of the particular disease undergoing therapy.

The amount of active ingredient that may be combined with the carrier materials to produce a single dosage form will vary depending upon the host treated and the particular mode of administration. For example, a formulation intended for the oral administration of humans may vary from about 5 to about 95% of the total composition.

A dosage unit (e.g. an oral dosage unit) can include from, for example, 0.01 to 0.1 mg, 1 to 30 mg, 1 to 40 mg, 1 to 100 mg, 1 to 300 mg, 1 to 500 mg, 2 to 500 mg, 3 to 100 mg, 5 to 20 mg, 5 to 100 mg (e.g. 0.01 mg, 1 mg, 2 mg, 3 mg, 4 mg, 5 mg, 6 mg, 7 mg, 8 mg, 9 mg, 10 mg, 11 mg, 12 mg, 13 mg, 14 mg, 15 mg, 16 mg, 17 mg, 18 mg, 19 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 150 mg, 200 mg, 250 mg, 300 mg, 350 mg, 400 mg, 450 mg, 500 mg) of a compound described herein.

The products described herein may be administered as frequently as necessary in order to obtain the desired therapeutic effect. Some patients may respond rapidly to a higher or lower dose and may find much weaker maintenance doses adequate. For other patients, it may be necessary to have long-term treatments at the rate of 1 to 4 doses per day, in accordance with the physiological requirements of each particular patient. Generally, the active product may be administered orally 1 to 4 times per day. It goes without saying that, for other patients, it will be necessary to prescribe not more than one or two doses per day.

For additional information about pharmaceutical compositions and their formulation, see, for example, Remington, The Science and Practice of Pharmacy, 20^{th} Edition (2000), which is hereby incorporated by reference in its entirety.

The compounds of formula 1 can be administered, e.g., by intravenous injection, intramuscular injection, subcutaneous injection, intraperitoneal injection, topical, sublingual, intraarticular (in the joints), intradermal, buccal, ophthalmic (including intraocular), intranasally (including using a cannula), or by other routes. The compounds of formula I can be administered orally, e.g., as a tablet or cachet containing a predetermined amount of the active ingredient, gel, pellet, paste, syrup, bolus, electuary, slurry, capsule, powder, granules, as a solution or a suspension in an aqueous liquid or a non-aqueous liquid, as an oil-in-water liquid emulsion or a water-in-oil liquid emulsion, via a micellar formulation (see, e.g. PCT Publication No. WO 97/11682, which is hereby incorporated by reference in its entirety) via a liposomal formulation (see, e.g., European Patent EP 736299 and PCT Publication Nos. WO 99/59550 and WO 97/13500, which are hereby incorporated by reference in their entirety), via formulations described in PCT Publication No. WO 03/094886, which is hereby incorporated by reference in its entirety, or in some other form. The compounds of formula I can also be administered transdermally (i.e. via reservoir-type or matrix-type patches, microneedles, thermal poration, hypodermic needles, iontophoresis, electroporation, ultrasound or other forms of sonophoresis, jet injection, or a combination of any of the preceding methods (Prausnitz et al., Nature Reviews Drug Discovery 3:115 (2004), which is hereby incorporated by reference in its entirety)). The compounds can be administered locally, for example, at the site of injury to an injured blood vessel. The compounds can be coated on a stent. The compounds can be administered using high-velocity transdermal particle injection techniques using the hydrogel particle formulation described in U.S. Patent Publication No. 20020061336, which is hereby incorporated by reference in its entirety. Additional particle formulations are described in PCT Publication Nos. WO 00/45792, WO 00/53160, and WO 02/19989, which are hereby incorporated by reference in their entirety. An example of a transdermal formulation containing plaster and the absorption promoter dimethylisosorbide can be found in PCT Publication No. WO 89/04179, which is hereby incorporated by reference in its entirety. PCT Publication No. WO 96/11705, which is hereby incorporated by reference in its entirety, provides formulations suitable for transdermal administration.

The compounds can be administered in the form a suppository or by other vaginal or rectal means. The compounds can be administered in a transmembrane formulation as described in PCT Publication No. WO 90/07923, which is hereby incorporated by reference in its entirety. The compounds can be administered non-invasively via the dehydrated particles described in U.S. Pat. No. 6,485,706, which is hereby incorporated by reference in its entirety. The compound can be administered in an enteric-coated drug formulation as described in PCT Publication No. WO 02/49621, which is hereby incorporated by reference in its entirety. The compounds can be administered intranasaly using the formulation described in U.S. Pat. No. 5,179,079, which is hereby incorporated by reference in its entirety. Formulations suitable for parenteral injection are described in PCT Publication No. WO 00/62759, which is hereby incorporated by reference in its entirety. The compounds can be administered using the casein formulation described in U.S. Patent Publication No. 20030206939 and PCT Publication No. WO 00/06108, which are hereby incorporated by reference in their entirety. The compounds can be administered using the particulate formulations described in U.S. Patent Application Publication No. 20020034536, which is hereby incorporated by reference in its entirety.

The compounds, alone or in combination with other suitable components, can be administered by pulmonary route utilizing several techniques including but not limited to intratracheal instillation (delivery of solution into the lungs by syringe), intratracheal delivery of liposomes, insufflation (administration of powder formulation by syringe or any other similar device into the lungs) and aerosol inhalation. Aerosols (e.g., jet or ultrasonic nebulizers, metered-dose inhalers (MDIs), and dry-Powder inhalers (DPIs)) can also be used in intranasal applications. Aerosol formulations are stable dispersions or suspensions of solid material and liquid droplets in a gaseous medium and can be placed into pressurized acceptable propellants, such as hydrofluoroalkanes (HFAs, i.e. HFA-134a and HFA-227, or a mixture thereof), dichlorodifluoromethane (or other chlorofluorocarbon propellants such as a mixture of Propellants 11, 12, and/or 114), propane, nitrogen, and the like. Pulmonary formulations may include permeation enhancers such as fatty acids, and saccharides, chelating agents, enzyme inhibitors (e.g., protease inhibitors), adjuvants (e.g., glycocholate, surfactin, span 85, and nafamostat), preservatives (e.g., benzalkonium chloride or chlorobutanol), and ethanol (normally up to 5% but possibly up to 20%, by weight). Ethanol is commonly included in aerosol compositions as it can improve the function of the metering valve and in some cases also improve the stability of the dispersion.

Pulmonary formulations may also include surfactants which include but are not limited to bile salts and those described in U.S. Pat. No. 6,524,557 and references therein, which is hereby incorporated by reference in its entirety. The surfactants described in U.S. Pat. No. 6,524,557, which is hereby incorporated by reference in its entirety, e.g., a C₈-C₁₆ fatty acid salt, a bile salt, a phospholipid, or alkyl saccharide are advantageous in that some of them also reportedly enhance absorption of the compound in the formulation.

Also suitable are dry powder formulations comprising a therapeutically effective amount of active compound blended with an appropriate carrier and adapted for use in connection with a dry-powder inhaler. Absorption enhancers that can be added to dry powder formulations described herein include those described in U.S. Pat. No. 6,632,456, which is hereby incorporated by reference in its entirety. PCT Publication No. WO 02/080884, which is hereby incorporated by reference in its entirety, describes new methods for the surface modification of powders. Aerosol formulations may include U.S. Pat. No. 5,230,884, U.S. Pat. No. 5,292,499, PCT Publication No. WO 017/8694, PCT Publication No. WO 01/78696, U.S. Patent Application Publication No. 2003019437, U.S. Patent Application Publication No. 20030165436, and PCT Publication No. WO 96/40089 (which includes vegetable oil), which are hereby incorporated by reference in their entirety. Sustained release formulations suitable for inhalation are described in U.S. Patent Application Publication Nos. 20010036481A1, 20030232019A1, and 20040018243A1 as well as in PCT Publication Nos. WO 01/13891, WO 02/067902, WO 03/072080, and WO 03/079885, which are hereby incorporated by reference in their entirety.

Pulmonary formulations containing microparticles are described in PCT Publication No. WO 03/015750, U.S. Patent Application Publication No. 20030008013, and PCT Publication No. WO 00/00176, which are hereby incorporated by reference in their entirety. Pulmonary formulations containing stable glassy state powder are described in U.S. Patent Application Publication No. 20020141945 and U.S. Pat. No. 6,309,671, which are hereby incorporated by reference in their entirety. Other aerosol formulations are described in EP 1338272A1, PCT Publication No. WO 90/09781, U.S. Pat. No. 5,348,730, U.S. Pat. No. 6,436,367, PCT Publication No. WO 91/04011, and U.S. Pat. No. 6,294,153, which are hereby incorporated by reference in their entirety, and U.S. Pat. No. 6,290,987, which is hereby incorporated by reference in its entirety, describes a liposomal based formulation that can be administered via aerosol or other means.

Powder formulations for inhalation are described in U.S. Patent Application Publication No. 20030053960 and PCT Publication No. WO 01/60341, which are hereby incorporated by reference in their entirety. The compounds can be administered intranasally as described in U.S. Patent Application Publication No. 20010038824, which is hereby incorporated by reference in its entirety.

Solutions of medicament in buffered saline and similar vehicles are commonly employed to generate an aerosol in a nebulizer. Simple nebulizers operate on Bernoulli's principle and employ a stream of air or oxygen to generate the spray particles. More complex nebulizers employ ultrasound to create the spray particles. Both types are well known in the art and are described in standard textbooks of pharmacy such as Sprowls' American Pharmacy and Remington's The Science and Practice of Pharmacy, which are hereby incorporated by reference in their entirety.

Other devices for generating aerosols employ compressed gases, usually hydrofluorocarbons and chlorofluorocarbons, which are mixed with the medicament and any necessary excipients in a pressurized container, these devices are likewise described in standard textbooks such as Sprowls and Remington, which are hereby incorporated by reference in their entirety.

Compounds of formula I can be incorporated into a liposome to improve half-life. Compounds of formula I can also be conjugated to polyethylene glycol (PEG) chains. Methods for pegylation and additional formulations containing PEG-conjugates (i.e. PEG-based hydrogels, PEG modified liposomes) can be found in Harris et al., Nature Reviews Drug Discovery, 2:214-221 (2003) and the references therein, which are hereby incorporated by reference in their entirety. Compounds of formula I can also be administered via a nanocochleate or cochleate delivery vehicle (BioDelivery Sciences International, Raleigh, N.C.). Compounds of formula I can also be delivered using nanoemulsion formulations.

### EXAMPLES

The Examples set forth below are for illustrative purposes only and are not intended to limit, in any way, the scope of the present invention.

### Example 1

### Analytical Methods and Materials

Unless otherwise noted, reagents and solvents were used as received from commercial suppliers. Proton nuclear magnetic resonance (NMR) spectra were obtained on Bruker spectrometers at 300, 400 or 500 MHz. Spectra are given in ppm (δ) and coupling constants, J, are reported in Hertz. Tetramethylsilane (TMS) was used as an internal standard. Mass spectra were collected using either a Finnigan LCQ Duo LCMS ion trap electrospray ionization (ESI) or a mass Varian 1200L single quadrapole mass spectrometer (ESI). High performance liquid chromatograph (HPLC) analyses were obtained using a Luna C18(2) column (250×4.6 mm, Phenomenex) or a Zorbax Bonus-RP column (150×4.6 mm, 3.5 um, Agilent) with UV detection at 254 nm or 223 nm using a standard solvent gradient program (Method A, Method B or Method C).

### Method A:

| Time | Flow | | |
|---|---|---|---|
| (min) | (mL/min) | % A | % B |
| 0.0 | 1.0 | 98 | 2 |
| 20 | 1.0 | 2 | 98 |
| 25 | 1.0 | 2 | 98 |
| 27 | 1.0 | 98 | 2 |

A = Water with 0.025% Trifluoroacetic Acid
B = Acetonitrile with 0.025% Trifluoroacetic Acid

### Method B:

| Time | Flow | | |
|---|---|---|---|
| (min) | (mL/min) | % A | % B |
| 0.0 | 1.2 | 98 | 2 |
| 6 | 1.2 | 79 | 21 |
| 12 | 1.2 | 76 | 24 |
| 22 | 1.2 | 0 | 100 |

A = Water with 0.1% Trifluoroacetic Acid
B = Acetonitrile with 0.1% Trifluoroacetic Acid

### Method C:

| Time | Flow | | |
|---|---|---|---|
| (min) | (mL/min) | % A | % B |
| 0.0 | 1.0 | 90 | 10 |
| 20 | 1.0 | 10 | 90 |
| 30 | 1.0 | 10 | 90 |
| 35 | 1.0 | 90 | 10 |

A = Water with 0.05% Trifluoroacetic Acid
B = Acetonitrile with 0.05% Trifluoroacetic Acid

### Example 2

### Preparation of 4-(Benzyloxy)-1-(1,2,3,4-tetrahydrobenzofuro[2,3-c]pyridin-7-yl)pyridin-2(1H)-one hydrochloride

### a) O-(3-Bromophenyl)hydroxylamine hydrochloride

CAS Registry Number 937716-47-7

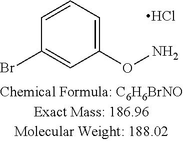

Potassium hydroxide (7.70 g, 137 mmol) was added to a biphasic solution of 3-bromophenol (19.0 g, 110 mmol) in toluene (27.0 mL), ⁱPrOH (16.4 mL) and H₂O (2.67 mL). The resulting suspension was heated at reflux for 1.5 h. A solution of hydroxylamine-O-sulfonic acid (3.10 g, 27.5 mmol) in H₂O (16.4 mL) was added dropwise over 20 min to the refluxing solution. The solution was allowed to stir for an additional 15 min. The solution was placed in an ice bath for 5 min. The solution was diluted with 10% NaOH (aq) (100 mL) and with CH₂Cl₂ (200 mL). The resulting layers were separated, and the aqueous phase was extracted with CH₂Cl₂ (2×75 mL). The combined organic extracts were washed with 10% NaOH (aq) (7×100 mL), dried over Na₂SO₄, filtered and partially concentrated under reduced pressure. The obtained solution was diluted with MeOH (18 mL), and 2 N HCl in Et₂O (18 mL) was added. The resulting solution was stirred at ambient temperature for 1 h. The solution was concentrated to provide the title compound (1.94 g, 31%) as red-brown solid: ¹H NMR (300 MHz, DMSO-d₆) δ 9.95 (br s, 3H), 7.40 (d, J=1.8 Hz, 1H), 7.31-7.23 (m, 1H), 7.20-7.09 (m, 2H); ESI MS m/z 188 [M+H]⁺.

### b) tert-Butyl 7-bromo-3,4-dihydrobenzofuro[2,3-c]pyridine-2(1H)-carboxylate

CAS Registry Number 201809-83-8

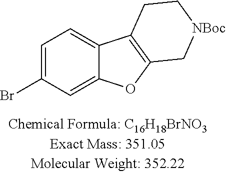 [SMILES: CC(C)(C)OC(=O)N1CCc2c(oc3cc(Br)ccc23)C1]

To a chilled solution of O-(3-bromophenyl)hydroxylamine hydrochloride (1.9 g, 8.7 mmol) in AcOH (8.0 mL) was added concentrated sulfuric acid (0.80 mL) followed by a slow addition of tert-butyl 3-oxopiperidine-1-carboxylate (2.1 g, 10 mmol). The resulting solution was stirred at ambient temperature for 5 min then heated to reflux and held at reflux for 3 h. The resulting solution was cooled in an ice bath and basified with 6 N NaOH. The resulting solution was extracted with CH₂Cl₂ (4×75 mL). The combined organic extracts were washed with brine, dried over Na₂SO₄, and concentrated under reduced pressure. The resulting crude material was diluted with 6:5 H₂O/iPrOH (31 mL) and treated with K₂CO₃ (1.4 g, 10 mmol) followed by Boc₂O (2.3 g, 10 mmol). The resulting solution was stirred at ambient temperature for 18 h, then diluted with H₂O (50 mL) and CH₂Cl₂ (100 mL). The resulting layers were separated, and the aqueous phase was extracted with CH₂Cl₂ (3×50 mL). The combined organic extracts were concentrated under reduced pressure. Flash chromatography (120 g ISCO column, hexanes/EtOAc, 95:5 for 2 min, increased to 50:50 over 40 min and held for 10 min) gave the title compound (0.47 g, 15%) as an off-white solid: ¹H NMR (500 MHz, DMSO-d₆) δ 7.85 (d, J=1.0 Hz, 1H), 7.50 (d, J=8.5 Hz, 1H), 7.42 (dd, J=8.5, 1.0 Hz, 1H), 4.54 (s, 2H), 3.66 (t, J=5.5 Hz, 2H), 2.67 (t, J=5.5 Hz, 2H), 1.43 (s, 9H).

### c) tert-Butyl 7-(4-(benzyloxy)-2-oxopyridin-1(2H)-yl)-3,4-dihydrobenzofuro[2,3-c]pyridine-2(1H)-carboxylate

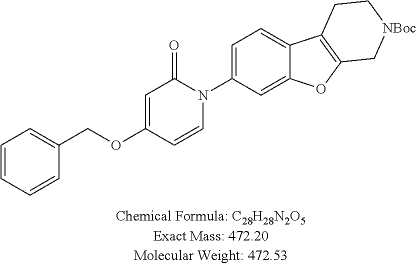 [SMILES: CC(C)(C)OC(=O)N1CCc2c(oc3cc(-n4ccc(OCc5ccccc5)cc4=O)ccc23)C1]

tert-Butyl 7-bromo-3,4-dihydrobenzofuro[2,3-c]pyridine-2(1H)-carboxylate (0.15 g, 0.43 mmol), 4-benzyloxy pyridinone (0.10 g, 0.51 mmol), and Cs₂CO₃ (0.18 g, 0.55 mmol) were suspended in toluene (6 mL), and N₂ was bubbled through the suspension for 30 min. The suspension was treated with (1S,2S)N,N′-bismethyl-1,2-cyclohexane-diamine (0.10 mL, 0.63 mmol) and bubbled with N₂ for 5 min. Copper iodide (0.12 g, 0.63 mmol) was added, and the resulting suspension was heated at reflux under N₂ for 18 h. The mixture was cooled to ambient temperature, diluted with 90:9:1 CH₂Cl₂/MeOH/NH₄OH (10 mL) and stirred for 20 min. A solution of 2:1 brine/NH₄OH (20 mL) was added followed by CH₂Cl₂ (30 mL), and the resulting layers were separated. The aqueous phase was extracted with CH₂Cl₂ (2×30 mL). The combined organic extracts were washed with 2:1 brine/NH₄OH (4×30 mL) and concentrated to dryness under reduced pressure. Flash chromatography (12 g ISCO column, CH₂Cl₂/(90:9:1 CH₂Cl₂/MeOH/NH₄OH), 100:0 for 3 min, increased to 0:100 over 30 min and held for 3 min) gave the title compound (0.18 g, 88%) as a light pink solid: ¹H NMR (500 MHz, DMSO-d₆) δ 7.62-7.58 (m, 3H), 7.48-7.40 (m, 4H), 7.39-7.34 (m, 1H), 7.22 (d, J=8.5 Hz, 1H), 6.11 (dd, J=7.5, 2.5 Hz, 1H), 5.98 (d, J=2.5 Hz, 1H), 5.14 (s, 2H), 4.58 (s, 2H), 3.68 (t, J=5.5 Hz, 2H) 2.76-2.69 (m, 2H), 1.44 (s, 9H); ESI MS m/z 473 [M+H]⁺.

### d) 4-(Benzyloxy)-1-(1,2,3,4-tetrahydrobenzofuro[2,3-c]pyridin-7-yl)pyridin-2(1H)-one

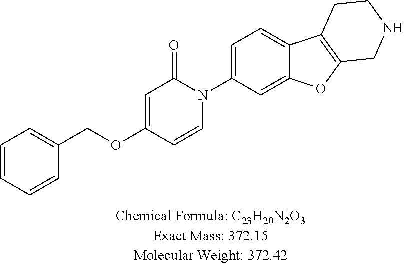 [SMILES: O=c1cc(OCc2ccccc2)ccn1-c1ccc2c3c(oc2c1)CNCC3]

A solution of tert-butyl 7-(4-(benzyloxy)-2-oxopyridin-1(2H)-yl)-3,4-dihydrobenzofuro[2,3-c]pyridine-2(1H)-carboxylate (0.17 g, 0.37 mmol) in MeOH (2.5 mL) was treated with 2 N HCl in Et₂O (0.45 mL), and the resulting solution was stirred at ambient temperature for 18 h. Additional 2 N HCl in Et₂O (0.60 mL) was added, and the resulting solution was stirred at ambient temperature for 3 h. The reaction was concentrated and suspended in CH₂Cl₂ (20 mL). The resulting suspension was treated with saturated NaHCO₃ solution (30 mL), and the resulting layers were separated. The aqueous phase was extracted with CH₂Cl₂ (2×30 mL), and the combined organic extracts were concentrated to dryness under reduced pressure. Flash chromatography (12 g ISCO column, CH₂Cl₂/(90:9:1 CH₂Cl₂/MeOH/NH₄OH), 100:0 for 2 min, increased to 0:100 over 25 min and held for 25 min) provided the title compound (97 mg, 71%) as a white solid: ¹H NMR (500 MHz, DMSO-d₆) δ 7.60-7.54 (m, 3H), 7.48-7.40 (m, 4H), 7.39-7.37 (m, 1H), 7.17 (dd, J=8.0, 1.5 Hz, 1H), 6.10 (dd, J=7.5, 2.5 Hz, 1H), 5.98 (d, J=2.5 Hz, 1H), 5.14 (s, 2H), 3.85 (s, 2H), 2.96 (t, J=5.5 Hz, 2H), 2.64-2.58 (m, 2H); ESI MS m/z 373 [M+H]⁺.

### e) 4-(Benzyloxy)-1-(1,2,3,4-tetrahydrobenzofuro[2,3-c]pyridin-7-yl)pyridin-2(1H)-one hydrochloride

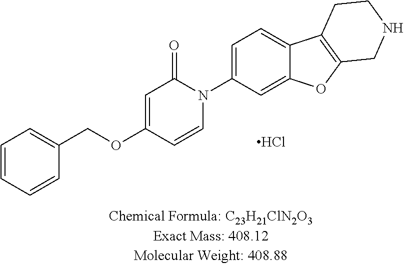 [SMILES: O=c1cc(OCc2ccccc2)ccn1-c1ccc2c3c(oc2c1)CNCC3 ; Cl]

A solution of 4-(benzyloxy)-1-(1,2,3,4-tetrahydrobenzofuro[2,3-c]pyridin-7-yl)pyridin-2(1H)-one (97 mg, 0.26 mmol) in MeOH (2.0 mL) was treated with 2 N HCl in Et₂O (0.13 mL, 0.26 mmol), and the resulting suspension was stirred at ambient temperature for 30 min. The suspension was concentrated to provide the title compound (98 mg, 93%) as an off-white solid: 285-293° C. decomp.; ¹H NMR (500 MHz, DMSO-d₆) δ 9.83 (br s, 2H), 7.70-7.68 (m, 2H), 7.60 (d, J=6.5 Hz, 1H), 7.48-7.40 (m, 4H), 7.39-7.36 (m, 1H), 7.30-7.27 (m, 1H), 6.12 (dd, J=7.5, 2.5 Hz, 1H), 5.99 (d, J=2.5 Hz, 1H), 5.15 (s, 2H), 4.43 (s, 2H), 3.46 (t, J=5.5 Hz, 2H), 2.96 (t, J=5.5 Hz, 2H); ESI MS m/z 373 [M+H]⁺; HPLC (Method A)>99% (AUC), t_{R}=13.50 min.

### Example 3

### Preparation of 4-((5-Fluoropyridin-2-yl)methoxy)-1-(1,2,3,4-tetrahydrobenzofuro[2,3-c]pyridin-7-yl)pyridin-2(1H)-one hydrochloride

### a) 4-((5-Fluoropyridin-2-yl)methoxy)pyridin-2(1H)-one

CAS Registry Number 924311-90-0

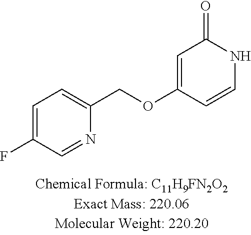 [SMILES: O=c1cc(OCc2ccc(F)cn2)cc[nH]1]

This compound was prepared in accordance with the procedure described in PCT Publication No. WO 2009/089482 to Guzzo et al., which is hereby incorporated by reference in its entirety.

### b) tert-Butyl 7-(4-((5-fluoropyridin-2-yl)methoxy)-2-oxopyridin-1(2H)-yl)-3,4-dihydrobenzofuro[2,3-c]pyridine-2(1H)-carboxylate

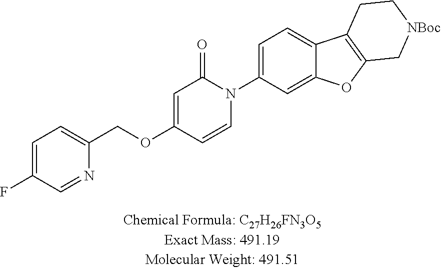 [SMILES: CC(C)(C)OC(=O)N1CCc2c(oc3cc(-n4ccc(OCc5ccc(F)cn5)cc4=O)ccc23)C1]

tert-Butyl 7-bromo-3,4-dihydrobenzofuro[2,3-c]pyridine-2(1H)-carboxylate (0.15 g, 0.43 mmol), 4-((5-fluoropyridin-2-yl)methoxy)pyridin-2(1H)-one (0.11 g, 0.51 mmol), and Cs₂CO₃ (0.18 g, 0.55 mmol) were suspended in toluene (6 mL), and N₂ was bubbled through the suspension for 15 min. The suspension was treated with (1S,2S)N,N′-bismethyl-1,2-cyclohexane-diamine (0.10 mL, 0.63 mmol) and bubbled with N₂ for 5 min. Copper iodide (0.12 g, 0.63 mmol) was added, and the resulting suspension was heated at reflux under N₂ for 18 h. The mixture was cooled to ambient temperature and diluted with a solution of 2:1 brine/NH₄OH (30 mL), and the resulting suspension was stirred for 30 min. The suspension was diluted with CH₂Cl₂ (30 mL), and the resulting biphasic solution was stirred for 15 min. The layers were separated, and the aqueous phase was extracted with CH₂Cl₂ (2×30 mL). The combined organic extracts were washed with 2:1 brine/NH₄OH (4×25 mL), dried over Na₂SO₄, filtered and concentrated to dryness under reduced pressure. Flash chromatography (12 g ISCO column, CH₂Cl₂/(90:9:1 CH₂Cl₂/MeOH/NH₄OH), 100:0 for 3 min, increased to 0:100 over 30 min and held for 3 min) gave the title compound (0.16 g, 78%) as an off-white foam: ¹H NMR (500 MHz, DMSO-d₆) δ 8.61 (d, J=3.0 Hz, 1H), 7.83-7.79 (m, 1H), 7.67-7.64 (m, 1H), 7.62-7.58 (m, 3H), 7.22 (dd, J=8.5, 1.5 Hz, 1H), 6.14 (dd, J=7.5, 2.5 Hz, 1H), 5.98 (d, J=3.0 Hz, 1H), 5.21 (s, 2H), 4.58 (s, 2H), 3.68 (t, J=5.5 Hz, 2H), 2.73-2.69 (m, 2H), 1.44 (s, 9H); ESI MS m/z 492 [M+H]⁺.

### c) 4-((5-Fluoropyridin-2-yl)methoxy)-1-(1,2,3,4-tetrahydrobenzofuro[2,3-c]pyridin-7-yl)pyridin-2(1H)-one

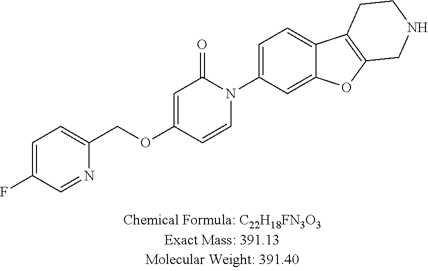 [SMILES: O=c1cc(OCc2ccc(F)cn2)ccn1-c1ccc2c3c(oc2c1)CNCC3]

A solution of tert-butyl 7-(4-((5-fluoropyridin-2-yl)methoxy)-2-oxopyridin-1(2H)-yl)-3,4-dihydrobenzofuro[2,3-c]pyridine-2(1H)-carboxylate (0.16 g, 0.33 mmol) in MeOH (2.0 mL) was treated with 2 N HCl in Et₂O (1.2 mL), and the resulting solution was stirred at ambient temperature for 18 h. The solution was diluted with CH₂Cl₂ (30 mL). The resulting solution was treated with saturated NaHCO₃ solution until the solution was basic. The resulting layers were separated, and the aqueous phase was extracted with CH₂Cl₂ (3×30 mL). The combined organic extracts were washed with 1:1 brine/saturated NaHCO₃ solution (30 mL) and concentrated to dryness under reduced pressure. Flash chromatography (12 g ISCO column, (CH₂Cl₂/(90:9:1 CH₂Cl₂/MeOH/NH₄OH), 100:0 for 1 min, increased to 0:100 over 25 min and held for 25 min) provided the title compound (0.12 g, 91%) as a white powder: ¹H NMR (500 MHz, DMSO-d₆) δ 8.61 (d, J=2.5 Hz, 1H), 7.83-7.80 (m, 1H), 7.67-7.64 (m, 1H), 7.62-7.59 (m, 1H), 7.57-7.54 (m, 2H), 7.17 (dd, J=8.5, 2.0 Hz, 1H), 6.13 (dd, J=7.5, 2.5 Hz, 1H), 5.98 (d, J=3.0 Hz, 1H), 5.21 (s, 2H), 3.85 (s, 2H), 2.96 (t, J=5.5 Hz, 2H), 2.61 (t, J=5.5 Hz, 2H); ESI MS m/z 392 [M+H]⁺; HPLC (Method A)>99% (AUC), t_{R}=12.0 min.

### d) 4-((5-Fluoropyridin-2-yl)methoxy)-1-(1,2,3,4-tetrahydrobenzofuro[2,3-c]pyridin-7-yl)pyridin-2(1H)-one hydrochloride

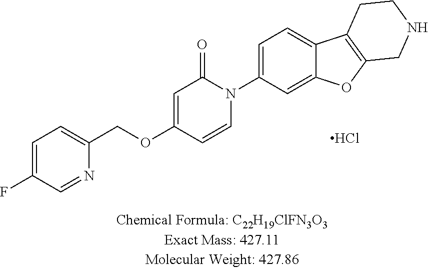 [SMILES: O=c1cc(OCc2ccc(F)cn2)ccn1-c1ccc2c3c(oc2c1)CNCC3 ; Cl]

A solution of 4-((5-fluoropyridin-2-yl)methoxy)-1-(1,2,3,4-tetrahydrobenzofuro[2,3-c]pyridin-7-yl)pyridin-2(1H)-one (0.12 g, 0.30 mmol) in MeOH (2.0 mL) was treated with 2 N HCl in Et₂O (0.14 mL, 0.30 mmol), and the resulting suspension was stirred at ambient temperature for 18 h. The suspension was concentrated to provide the title compound (0.12 g, 92%) as a white solid: 268-280° C. decomp.; ¹H NMR (500 MHz, DMSO-d₆) δ 9.58 (br s, 2H), 8.62 (d, J=2.5 Hz, 1H), 7.85-7.80 (m, 1H), 7.71-7.68 (m, 2H), 7.67-7.64 (m, 1H), 7.63-7.60 (m, 1H), 7.28 (dd, J=8.5, 2.0 Hz, 1H), 6.16 (dd, J=7.5, 2.5 Hz, 1H), 5.99 (d, J=2.5 Hz, 1H), 5.22 (s, 2H), 4.44 (s, 2H), 3.47 (t, J=6.0 Hz, 2H), 2.96 (t, J=5.5 Hz, 2H); ESI MS m/z 392 [M+H]⁺; HPLC (Method A)>99% (AUC), t_{R}=12.0 min.

### Example 4

### Preparation of 1′-(1,2,3,4-Tetrahydrobenzofuro[2,3-c]pyridin-7-yl)-6-(trifluoromethyl)-[3,4′-bipyridin]-2′(1′H)-one hydrochloride

### a) 4-(6-(Trifluoromethyl)pyridin-3-yl)pyridin-2(1H)-one

CAS Registry Number 1173155-81-1

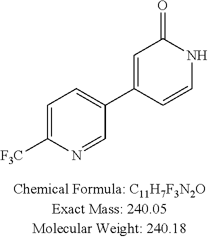 [SMILES: O=c1cc(-c2ccc(C(F)(F)F)nc2)cc[nH]1]

This compound was prepared in accordance with the procedure described in PCT Publication No. WO 2009/089482 to Guzzo et al., which is hereby incorporated by reference in its entirety.

### b) tert-Butyl 7-(2′-oxo-6-(trifluoromethyl)-[3,4′-bipyridin]-1′(2′H)-yl)-3,4-dihydrobenzofuro[2,3-c]pyridine-2(1H)-carboxylate

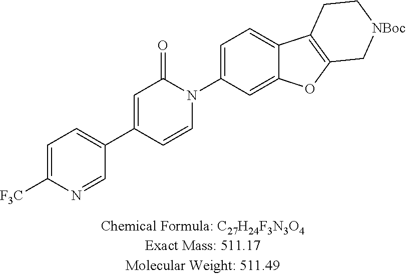 [SMILES: CC(C)(C)OC(=O)N1CCc2c(oc3cc(-n4ccc(-c5ccc(C(F)(F)F)nc5)cc4=O)ccc23)C1]

tert-Butyl 7-bromo-3,4-dihydrobenzofuro[2,3-c]pyridine-2(1H)-carboxylate (0.10 g, 0.28 mmol), 4-(6-(trifluoromethyl)pyridin-3-yl)pyridin-2(1H)-one (82 mg, 0.34 mmol), and Cs₂CO₃ (0.12 g, 0.37 mmol) were suspended in toluene (4 mL), and N₂ was bubbled through the suspension for 15 min. The suspension was treated with (1S,2S)N,N′-bismethyl-1,2-cyclohexane-diamine (67 μL, 0.43 mmol) and bubbled with N₂ for 5 min. Copper iodide (81 mg, 0.43 mmol) was added, and the resulting suspension was heated at reflux under N₂ for 18 h. The mixture was cooled to ambient temperature and diluted with a solution of 1:1 brine/NH₄OH (30 mL) followed by CH₂Cl₂ (75 mL). The layers were separated, and the aqueous phase was extracted with CH₂Cl₂ (3×30 mL). The combined organic extracts were washed with 1:1 brine/NH₄OH (3×40 mL) and concentrated to dryness under reduced pressure. Flash chromatography (12 g ISCO Gold column, CH₂Cl₂/(90:9:1 CH₂Cl₂/MeOH/NH₄OH), 100:0 for 2 min, increased to 50:50 over 30 min and held for 10 min) gave the title compound (0.12 g, 79%) as yellow solid: ¹H NMR (500 MHz, DMSO-d₆) δ 9.19 (d, J=2.0 Hz, 1H), 8.49 (dd, J=8.0, 2.0 Hz, 1H), 8.05 (d, J=8.0 Hz, 1H), 7.88 (d, J=7.0 Hz, 1H), 7.74 (d, J=2.0 Hz, 1H), 7.66 (d, J=8.0 Hz, 1H), 7.33 (dd, J=8.0, 1.5 Hz, 1H), 7.02 (d, J=2.0 Hz, 1H), 6.81 (dd, J=7.0, 2.0 Hz, 1H), 4.60 (s, 2H), 3.70 (t, J=5.5 Hz, 2H), 2.75-2.72 (m, 2H), 1.45 (s, 9H); ESI MS m/z 512 [M+H]⁺.

### c) 1′-(1,2,3,4-Tetrahydrobenzofuro[2,3-c]pyridin-7-yl)-6-(trifluoromethyl)-[3,4′-bipyridin]-2′(1′H)-one

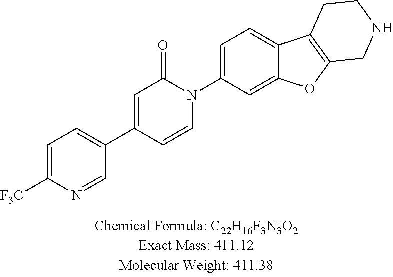 [SMILES: O=c1cc(-c2ccc(C(F)(F)F)nc2)ccn1-c1ccc2c3c(oc2c1)CNCC3]

A solution of tert-butyl 7-(2′-oxo-6-(trifluoromethyl)-[3,4′-bipyridin]-1′(2′H)-yl)-3,4-dihydrobenzofuro[2,3-c]pyridine-2(1H)-carboxylate (0.12 g, 0.22 mmol) in MeOH (2.0 mL) was treated with 2 N HCl in Et₂O (0.27 mL), and the resulting solution was stirred at ambient temperature for 18 h. Additional 2 N HCl in Et₂O (0.50 mL) was added, and the resulting solution was stirred at ambient temperature for 3 h. Additional 2 N HCl in Et₂O (0.50 mL) was added, and the resulting solution was stirred and ambient temperature for 18 h. The reaction was concentrated and suspended in CH₂Cl₂ (30 mL). The resulting solution was treated with saturated NaHCO₃ solution (20 mL), and the resulting layers were separated. The aqueous phase was extracted with CH₂Cl₂ (4×30 mL), and the combined organic extracts were concentrated to dryness under reduced pressure. Flash chromatography (12 g ISCO column, CH₂Cl₂/(90:9:1 CH₂Cl₂/MeOH/NH₄OH), 100:0 for 1 min, increased to 0:100 over 30 min) gave the title compound (69 mg, 74%) as a yellow powder: ¹H NMR (500 MHz, DMSO-d₆) δ 9.19 (d, J=2.0 Hz, 1H), 8.49 (dd, J=8.0, 2.0 Hz, 1H), 8.05 (d, J=8.0 Hz, 1H), 7.87 (d, J=7.0 Hz, 1H), 7.68 (d, J=1.5 Hz, 1H), 7.61 (d, J=8.5 Hz, 1H), 7.29 (dd, J=8.0, 1.5 Hz, 1H), 7.02 (d, J=2.0 Hz, 1H), 6.81 (dd, J=7.0, 2.0 Hz, 1H), 3.88 (s, 2H), 2.99 (t, J=5.5 Hz, 2H), 2.67-2.62 (m, 2H); HPLC (Method A)>99% (AUC), t_{R}=13.1 min.

### d) 1′-(1,2,3,4-Tetrahydrobenzofuro[2,3-c]pyridin-7-yl)-6-(trifluoromethyl)-[3,4′-bipyridin]-2′(1′H)-one hydrochloride

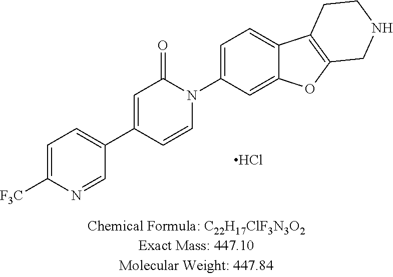 [SMILES: O=c1cc(-c2ccc(C(F)(F)F)nc2)ccn1-c1ccc2c3c(oc2c1)CNCC3 ; Cl]

A solution of 1′-(1,2,3,4-tetrahydrobenzofuro[2,3-c]pyridin-7-yl)-6-(trifluoromethyl)-[3,4′-bipyridin]-2′(1′H)-one (67 mg, 0.16 mmol) in MeOH (2.0 mL) was treated with 2 N HCl in Et₂O (81 μL, 0.16 mmol), and the resulting suspension was stirred at ambient temperature for 30 min. The suspension was concentrated to provide the title compound (72 mg, quant.) as a light yellow solid: 306-309° C. decomp.; ¹H NMR (500 MHz, DMSO-d₆) δ 9.81 (br s, 2H), 9.20 (d, J=2.0 Hz, 1H), 8.50 (dd, J=8.5, 2.0 Hz, 1H), 8.05 (d, J=8.0 Hz, 1H), 7.89 (d, J=7.0 Hz, 1H), 7.82 (d, J=1.5 Hz, 1H), 7.75 (d, J=8.5 Hz, 1H), 7.39 (dd, J=8.0, 2.0 Hz, 1H), 7.03 (d, J=2.0 Hz, 1H), 6.83 (dd, J=7.5, 2.5 Hz, 1H), 4.45 (s, 2H), 3.48 (t, J=6.0 Hz, 2H), 3.02-2.97 (m, 2H); ESI MS m/z 412 [M+H]⁺; HPLC (Method A) 99.0% (AUC), t_{R}=13.1 min.

### Example 5

### Preparation of 1′-(1,2,3,4-Tetrahydrobenzofuro[3,2-c]pyridin-7-yl)-6-(trifluoromethyl)-[3,4′-bipyridin]-2′(1′H)-one hydrochloride

### a) tert-Butyl 7-(2′-oxo-6-(trifluoromethyl)-[3,4′-bipyridin]-1′(2′H)-yl)-3,4-dihydrobenzofuro[3,2-c]pyridine-2(1H)-carboxylate

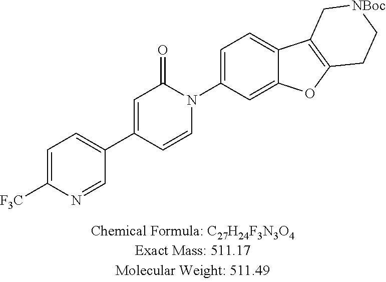 [SMILES: CC(C)(C)OC(=O)N1CCc2oc3cc(-n4ccc(-c5ccc(C(F)(F)F)nc5)cc4=O)ccc3c2C1]

tert-Butyl 7-bromo-3,4-dihydrobenzofuro[3,2-c]pyridine-2(1H)-carboxylate (0.10 g, 0.28 mmol, prepared according to Example 10, step b), 4-(6-(trifluoromethyl)pyridin-3-yl)pyridin-2(1H)-one (82 mg, 0.34 mmol), and Cs₂CO₃ (0.12 g, 0.37 mmol) were suspended in toluene (4.0 mL), and N₂ was bubbled through the suspension for 15 min. The suspension was treated with (1S,2S)N,N′-bismethyl-1,2-cyclohexane-diamine (67 μL, 0.43 mmol) and bubbled with N₂ for 5 min. Copper iodide (81 mg, 0.43 mmol) was added, and the resulting suspension was heated at reflux under N₂ for 18 h. The mixture was cooled to ambient temperature and diluted with a solution of 1:1 brine/NH₄OH (30 mL) followed by CH₂Cl₂ (75 mL). The layers were separated, and the aqueous phase was extracted with CH₂Cl₂ (3×50 mL). The combined organic extracts were washed with 1:1 brine/NH₄OH (4×30 mL) and concentrated to dryness under reduced pressure. Flash chromatography (12 g ISCO Gold column, CH₂Cl₂/(90:9:1 CH₂Cl₂/MeOH/NH₄OH), 100:0 for 2 min, increased to 50:50 over 30 min and held for 10 min) gave the title compound (84 mg, 57%) as yellow solid: ¹H NMR (500 MHz, DMSO-d₆) δ 9.19 (d, J=2.0 Hz, 1H), 8.49 (dd, J=8.0, 2.0 Hz, 1H), 8.05 (d, J=8.0 Hz, 1H), 7.88 (d, J=7.5 Hz, 1H), 7.73 (d, J=2.0 Hz, 1H), 7.70 (d, J=8.5 Hz, 1H), 7.31 (dd, J=8.5, 2.0 Hz, 1H), 7.02 (d, J=2.0 Hz, 1H), 6.81 (dd, J=7.0, 2.0 Hz, 1H), 4.56 (s, 2H), 3.77 (t, J=5.5 Hz, 2H), 2.87 (t, J=5.5 Hz, 2H), 1.45 (s, 9H); ESI MS m/z 512 [M+H]⁺.

### b) 1′-(1,2,3,4-Tetrahydrobenzofuro[3,2-c]pyridin-7-yl)-6-(trifluoromethyl)-[3,4′-bipyridin]-2′(1′H)-one

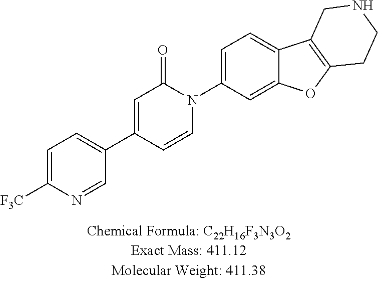 [SMILES: O=c1cc(-c2ccc(C(F)(F)F)nc2)ccn1-c1ccc2c3c(oc2c1)CCNC3]

A solution of tert-butyl 7-(2′-oxo-6-(trifluoromethyl)-[3,4′-bipyridin]-1′(2′H)-yl)-3,4-dihydrobenzofuro[3,2-c]pyridine-2(1H)-carboxylate (80 mg, 0.16 mmol) in MeOH (1.5 mL) was treated with 2 N HCl in Et₂O (0.18 mL), and the resulting solution was stirred at ambient temperature for 18 h. Additional 2 N HCl in Et₂O (0.50 mL) was added, and the resulting solution was stirred at ambient temperature for 3 h. Additional 2 N HCl in Et₂O (0.50 mL) was added, and the resulting solution was stirred at ambient temperature for 18 h. The reaction was concentrated and suspended in CH₂Cl₂ (30 mL). The resulting solution was treated with saturated NaHCO₃ solution (20 mL), and the resulting layers were separated. The aqueous phase was extracted with CH₂Cl₂ (4×30 mL), and the combined organic extracts were concentrated to dryness under reduced pressure to provide the title compound (48 mg, 75%) as a yellow solid: ¹H NMR (500 MHz, DMSO-d₆) δ 9.19 (d, J=2.0 Hz, 1H), 8.49 (dd, J=8.5, 2.0 Hz, 1H), 8.05 (d, J=8.5 Hz, 1H), 7.87 (d, J=7.0 Hz, 1H), 7.67 (d, J=1.5 Hz, 1H), 7.58 (d, J=8.5 Hz, 1H), 7.27 (dd, J=8.0, 2.0 Hz, 1H), 7.01 (d, J=2.0 Hz, 1H), 6.80 (dd, J=7.5, 2.0 Hz, 1H), 3.84 (s, 2H), 3.07 (t, J=5.5 Hz, 2H), 2.76-2.72 (m, 2H); HPLC (Method A)>99% (AUC), t_{R}=13.1 min.

### c) 1′-(1,2,3,4-Tetrahydrobenzofuro[3,2-c]pyridin-7-yl)-6-(trifluoromethyl)-[3,4′-bipyridin]-2′(1′H)-one hydrochloride

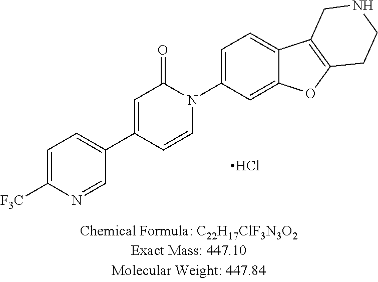 [SMILES: O=c1cc(-c2ccc(C(F)(F)F)nc2)ccn1-c1ccc2c3c(oc2c1)CCNC3 ; Cl]

A solution of 1′-(1,2,3,4-tetrahydrobenzofuro[3,2-c]pyridin-7-yl)-6-(trifluoromethyl)-[3,4′-bipyridin]-2′(1′H)-one (45 mg, 0.11 mmol) in MeOH (2.0 mL) was treated with 2 N HCl in Et₂O (54 μL, 0.11 mmol), and the resulting suspension was stirred at ambient temperature for 30 min. The suspension was concentrated to provide the title compound (61 mg, quant.) as a white solid: 300-315° C. decomp.; ¹H NMR (500 MHz, DMSO-d₆) δ 9.62 (br s, 2H), 9.20 (d, J=2.0 Hz, 1H), 8.51 (dd, J=8.0, 2.0 Hz, 1H), 8.05 (d, J=8.0 Hz, 1H), 7.88 (d, J=7.5 Hz, 1H), 7.80 (d, J=2.0 Hz, 1H), 7.74 (d, J=8.5 Hz, 1H), 7.38 (dd, J=8.5, 2.0 Hz, 1H), 7.03 (d, J=2.0 Hz, 1H), 6.83 (dd, J=7.5, 2.0 Hz, 1H), 4.37 (s, 2H), 3.56 (t, J=6.0 Hz, 2H), 3.16-3.12 (m, 2H); ESI MS m/z 412 [M+H]⁺; HPLC (Method A)>99% (AUC), t_{R}=13.2 min.

### Example 6

### Preparation of 4-(Benzyloxy)-1-(1,2,3,4-tetrahydrobenzofuro[3,2-c]pyridin-7-yl)pyridin-2(1H)-one hydrochloride

### a) tert-Butyl 7-(4-(benzyloxy)-2-oxopyridin-1(2H)-yl)-3,4-dihydrobenzofuro[3,2-c]pyridine-2(1H)-carboxylate

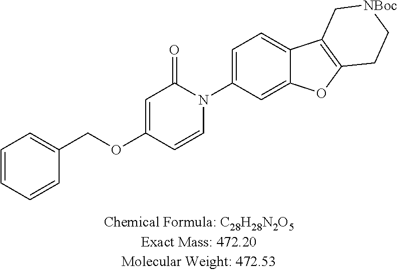 [SMILES: CC(C)(C)OC(=O)N1CCc2oc3cc(-n4ccc(OCc5ccccc5)cc4=O)ccc3c2C1]

tert-Butyl 7-bromo-3,4-dihydrobenzofuro[3,2-c]pyridine-2(1H)-carboxylate (0.15 g, 0.42 mmol), 4-benzyloxy pyridinone (0.10 g, 0.51 mmol), and Cs₂CO₃ (0.18 g, 0.55 mmol) were suspended in toluene (15 mL), and N₂ was bubbled through the suspension for 20 min. The suspension was treated with (1S,2S)N,N′-bismethyl-1,2-cyclohexane-diamine (0.10 mL, 0.63 mmol) and bubbled with N₂ for 5 min. Copper iodide (0.12 g, 0.63 mmol) was added, and the resulting suspension was heated at reflux under N₂ for 18 h. The mixture was cooled, diluted with 90:9:1 CH₂Cl₂/MeOH/NH₄OH (15 mL) and stirred for 30 min. A solution of 2:1 brine/NH₄OH (30 mL) was added, and the solution was extracted with CH₂Cl₂ (3×50 mL). The combined organic extracts were washed with 2:1 brine/NH₄OH (3×50 mL), dried over Na₂SO₄, filtered and concentrated to dryness under reduced pressure. Flash chromatography (12 g ISCO column, CH₂Cl₂/(90:9:1 CH₂Cl₂/MeOH/NH₄OH), 100:0 for 3 min, increased to 50:50 over 25 min and held for 5 min; increased to 0:100 over 5 min and held for 3 min) gave the title compound (0.14 g, 68%) as a yellow film: ¹H NMR (500 MHz, DMSO-d₆) δ 7.64 (d, J=8.0 Hz, 1H), 7.61-7.58 (m, 2H), 7.48-7.41 (m, 4H), 7.38-7.37 (m, 1H), 7.20 (dd, J=8.0, 2.0 Hz, 1H), 6.11 (dd, J=7.5, 3.0 Hz, 1H), 5.98 (d, J=2.5 Hz, 1H), 5.15 (s, 2H), 4.53 (s, 2H), 3.76 (t, J=5.5 Hz, 2H) 2.85 (t, J=5.5 Hz, 2H), 1.44 (s, 9H); ESI MS m/z 473 [M+H]⁺.

### b) 4-(Benzyloxy)-1-(1,2,3,4-tetrahydrobenzofuro[3,2-c]pyridin-7-yl)pyridin-2(1H)-one

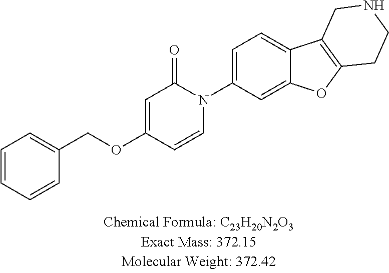 [SMILES: O=c1cc(OCc2ccccc2)ccn1-c1ccc2c3c(oc2c1)CCNC3]

A solution of tert-butyl 7-(4-(benzyloxy)-2-oxopyridin-1(2H)-yl)-3,4-dihydrobenzofuro[3,2-c]pyridine-2(1H)-carboxylate (0.14 g, 0.29 mmol) in MeOH (1.5 mL) was treated with 2 N HCl in Et₂O (0.9 mL), and the resulting solution was stirred at ambient temperature for 18 h. The reaction was concentrated and suspended in CH₂Cl₂ (20 mL). The resulting solution was treated with saturated NaHCO₃ solution (20 mL), and the resulting layers were separated. The aqueous phase was extracted with CH₂Cl₂ (3×50 mL), and the combined organic extracts were concentrated to dryness under reduced pressure. Flash chromatography (12 g ISCO column, CH₂Cl₂/(90:9:1 CH₂Cl₂/MeOH/NH₄OH), 100:0 for 1 min, increased to 0:100 over 25 min and held for 10 min) provided the title compound (55 mg, 51%) as an off-white solid: ¹H NMR (500 MHz, DMSO-d₆) δ 7.58 (d, J=7.5 Hz, 1H), 7.55 (d, J=1.5 Hz, 1H), 7.52 (d, J=8.0 Hz, 1H), 7.47-7.40 (m, 4H), 7.38-7.35 (m, 1H), 7.16 (d, J=8.5 Hz, 1H), 6.10 (dd, J=8.0, 3.0 Hz, 1H), 5.97 (d, J=2.5 Hz, 1H), 5.14 (s, 2H), 3.82 (s, 2H), 3.05 (t, J=5.5 Hz, 2H), 2.72 (t, J=5.5 Hz, 2H); ESI MS m/z 373 [M+H]⁺.

### c) 4-(Benzyloxy)-1-(1,2,3,4-tetrahydrobenzofuro[3,2-c]pyridin-7-yl)pyridin-2(1H)-one hydrochloride

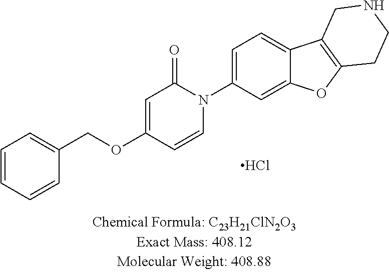 [SMILES: O=c1cc(OCc2ccccc2)ccn1-c1ccc2c3c(oc2c1)CCNC3 ; Cl]

A solution of 4-(benzyloxy)-1-(1,2,3,4-tetrahydrobenzofuro[3,2-c]pyridin-7-yl)pyridin-2(1H)-one (50 mg, 0.13 mmol) in MeOH (2.0 mL) was treated with 2 N HCl in Et₂O (67 μL, 0.13 mmol), and the resulting suspension was stirred at ambient temperature for 45 min. The suspension was concentrated to provide the title compound (54 mg, 99%) as an off-white powder: 291-294° C.; ¹H NMR (500 MHz, DMSO-d₆) δ 9.47 (br s, 2H), 7.70-7.67 (m, 2H), 7.59 (d, J=7.5 Hz, 1H), 7.48-7.47 (m, 2H), 7.46-7.41 (m, 2H), 7.39-7.35 (m, 1H), 7.27 (dd, J=8.0, 2.0 Hz, 1H), 6.12 (dd, J=7.5, 2.5 Hz, 1H), 5.99 (d, J=3.0 Hz, 1H), 5.15 (s, 2H), 4.35 (s, 2H), 3.59-3.52 (m, 2H), 3.11 (t, J=5.5 Hz, 2H); ESI MS m/z 373 [M+H]⁺; HPLC (Method A)>99% (AUC), t_{R}=13.5 min.

### Example 7

### Preparation of 4-((5-Fluoropyridin-2-yl)methoxy)-1-(1,2,3,4-tetrahydrobenzofuro[3,2-c]pyridin-7-yl)pyridin-2(1H)-one hydrochloride

### a) tert-Butyl 7-(4-((5-fluoropyridin-2-yl)methoxy)-2-oxopyridin-1(2H)-yl)-3,4-dihydrobenzofuro[3,2-c]pyridine-2(1H)-carboxylate

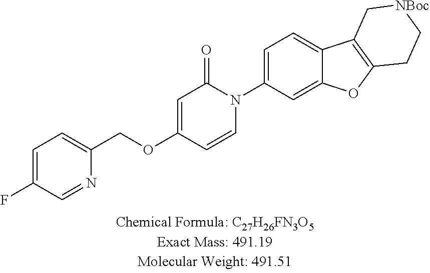 [SMILES: CC(C)(C)OC(=O)N1CCc2oc3cc(-n4ccc(OCc5ccc(F)cn5)cc4=O)ccc3c2C1]

tert-Butyl 7-bromo-3,4-dihydrobenzofuro[3,2-c]pyridine-2(1H)-carboxylate (0.15 g, 0.42 mmol), 4-((5-fluoropyridin-2-yl)methoxy)pyridin-2(1H)-one (0.11 g, 0.51 mmol), and Cs₂CO₃ (0.18 g, 0.55 mmol) were suspended in toluene (15 mL), and N₂ was bubbled through the suspension for 15 min. The suspension was treated with (1S,2S)N,N′-bismethyl-1,2-cyclohexane-diamine (0.10 mL, 0.63 mmol) and bubbled with N₂ for 5 min. Copper iodide (0.12 g, 0.63 mmol) was added, and the resulting suspension was heated at reflux under N₂ for 18 h. The mixture was cooled, diluted with 90:9:1 CH₂Cl₂/MeOH/NH₄OH (20 mL) and stirred for 1 h. A solution of 2:1 brine/NH₄OH (30 mL) was added, and the solution was stirred for 30 min. The solution was diluted with CH₂Cl₂ (100 mL), and the resulting layers were separated. The aqueous phase was extracted with CH₂Cl₂ (3×75 mL), and the combined organic extracts were washed with 2:1 brine/NH₄OH (3×100 mL) and concentrated to dryness under reduced pressure. Flash chromatography (12 g ISCO column, CH₂Cl₂/(90:9:1 CH₂Cl₂/MeOH/NH₄OH), 100:0 for 3 min, increased to 0:100 over 30 min and held for 5 min) gave the title compound (0.12 g, 58%) as an off-white foam: ¹H NMR (500 MHz, DMSO-d₆) δ 8.61 (d, J=3.0 Hz, 1H), 7.84-7.80 (m, 1H), 7.67-7.59 (m, 4H), 7.20 (dd, J=8.5, 2.0 Hz, 1H), 6.14 (dd, J=8.0, 2.5 Hz, 1H), 5.98 (d, J=2.5 Hz, 1H), 5.21 (s, 2H), 4.53 (s, 2H), 3.76 (t, J=5.5 Hz, 2H), 2.88-2.82 (m, 2H), 1.44 (s, 9H); ESI MS m/z 492 [M+H]⁺.

### b) 4-((5-Fluoropyridin-2-yl)methoxy)-1-(1,2,3,4-tetrahydrobenzofuro[3,2-c]pyridin-7-yl)pyridin-2(1H)-one

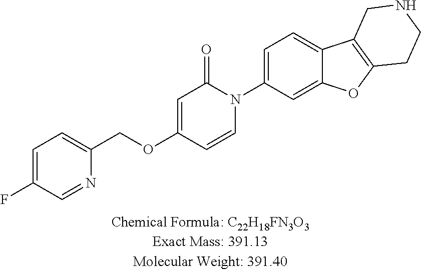 [SMILES: O=c1cc(OCc2ccc(F)cn2)ccn1-c1ccc2c3c(oc2c1)CCNC3]

A solution of tert-butyl 7-(4-((5-fluoropyridin-2-yl)methoxy)-2-oxopyridin-1(2H)-yl)-3,4-dihydrobenzofuro[3,2-c]pyridine-2(1H)-carboxylate (0.12 g, 0.24 mmol) in MeOH (1.5 mL) was treated with 2 N HCl in Et₂O (0.9 mL), and the resulting solution was stirred at ambient temperature for 18 h. The solution was diluted with CH₂Cl₂ (30 mL). The resulting solution was treated with saturated NaHCO₃ solution until the solution was basic. The resulting layers were separated, and the aqueous phase was extracted with CH₂Cl₂ (3×30 mL). The combined organic extracts were dried over Na₂SO₄, filtered and concentrated to dryness under reduced pressure. Flash chromatography (12 g ISCO column, CH₂Cl₂/(90:9:1 CH₂Cl₂/MeOH/NH₄OH), 100:0 for 1 min, increased to 0:100 over 25 min and held for 5 min) provided the title compound (78 mg, 82%) as an off-white foam: ¹H NMR (300 MHz, DMSO-d₆) δ 8.62 (d, J=3.0 Hz, 1H), 7.86-7.79 (m, 1H), 7.68-7.59 (m, 2H), 7.56-7.51 (m, 2H), 7.16 (dd, J=8.1, 1.8 Hz, 1H), 6.13 (dd, J=7.5, 2.7 Hz, 1H), 5.98 (d, J=2.7 Hz, 1H), 5.21 (s, 2H), 3.82 (s, 2H), 3.06 (t, J=5.7 Hz, 2H), 2.75-2.67 (m, 2H); ESI MS m/z 392 [M+H]⁺.

### c) 4-((5-Fluoropyridin-2-yl)methoxy)-1-(1,2,3,4-tetrahydrobenzofuro[3,2-c]pyridin-7-yl)pyridin-2(1H)-one hydrochloride

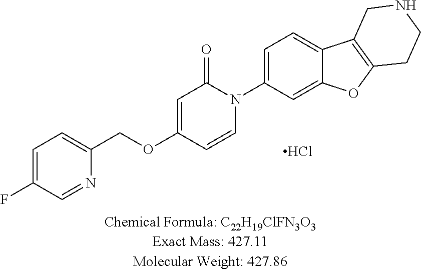 [SMILES: O=c1cc(OCc2ccc(F)cn2)ccn1-c1ccc2c3c(oc2c1)CCNC3 ; Cl]

A solution of 4-((5-fluoropyridin-2-yl)methoxy)-1-(1,2,3,4-tetrahydrobenzofuro[3,2-c]pyridin-7-yl)pyridin-2(1H)-one (77 mg, 0.20 mmol) in MeOH (2.0 mL) was treated with 2 N HCl in Et₂O (98 μL, 0.20 mmol), and the resulting suspension was stirred at ambient temperature for 3 h. The suspension was concentrated to provide the title compound (76 mg, 91%) as a white powder: ¹H NMR (500 MHz, DMSO-d₆) δ 8.61 (d, J=2.5 Hz, 1H), 7.84-7.80 (m, 1H), 7.66-7.64 (m, 1H), 7.61-7.54 (m, 3H), 7.19 (dd, J=8.0, 2.0 Hz, 1H), 6.14 (dd, J=7.5, 2.5 Hz, 1H), 5.98 (d, J=2.5 Hz, 1H), 5.21 (s, 2H), 3.97 (s, 2H), 3.18 (t, J=5.5 Hz, 2H), 2.81 (t, J=5.5 Hz, 2H); ESI MS m/z 392 [M+H]⁺; HPLC (Method A)>99% (AUC), t_{R}=12.0 min.

### Example 8

### Preparation of (R)-1-(3-Methyl-1,2,3,4-tetrahydrobenzofuro[3,2-c]pyridin-7-yl)-4-(pyridin-2-ylmethoxy)pyridin-2(1H)-one hydrochloride

### a) 4-(Pyridin-2-ylmethoxy)pyridin-2(1H)-one

CAS Registry Number 1008518-20-4

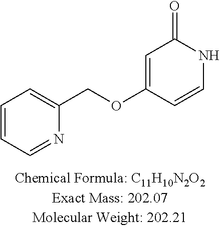 [SMILES: O=c1cc(OCc2ccccn2)cc[nH]1]

This compound was prepared in accordance with the procedure described in PCT Publication No. WO 2009/089482 to Guzzo et al., which is hereby incorporated by reference in its entirety.

### b) (R)-7-Bromo-3-methyl-1,2,3,4-tetrahydrobenzofuro[3,2-c]pyridine

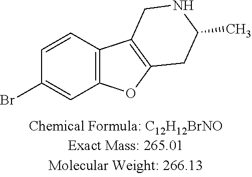

To a mixture of O-(3-bromophenyl)hydroxylamine hydrochloride (1.01 g, 4.49 mmol) and (R)-2-methylpiperidin-4-one acetic acid salt (1.93 g, 6.58 mmol) in acetic acid (10 mL) was added sulfuric acid (0.5 mL). This mixture was heated at 100-110° C. for 1.5 h. The reaction mixture was allowed to cool, quenched with saturated NaHCO₃ solution, and extracted with CH₂Cl₂ (250 mL). The extracts were washed with brine (250 mL), dried over Na₂SO₄, and concentrated under reduced pressure to afford the title compound (1.22 g, crude) as a light brown solid and as a mixture of regiosiomers. This material was used in the next step without further purification: ¹H NMR (500 MHz, CDCl₃) δ 7.59 (m, 1.3H), 7.32 (m, 3H), 7.22 (d, J=8.2 Hz, 1H), 7.06 (m, 1.6H), 4.18 (m, 1H), 3.99 (m, 2H), 3.38 (m, 1H), 3.16 (m, 2.6H), 2.79 (m, 2H), 2.70 (m, 1H), 2.47 (m, 1.5H), 1.59 (d, J=6.6 Hz, 1H), 1.49 (d, J=6.8 Hz, 1.8H), 1.33 (d, J=6.4 Hz, 4.2H).

### c) (R)-1-(3-Methyl-1,2,3,4-tetrahydrobenzofuro[3,2-c]pyridin-7-yl)-4-(pyridin-2-ylmethoxy)pyridin-2(1H)-one

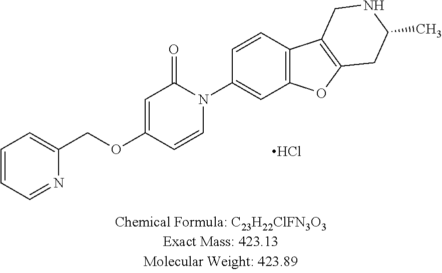 [SMILES: C[C@@H]1Cc2oc3cc(-n4ccc(OCc5ccccn5)cc4=O)ccc3c2CN1 ; Cl]

A mixture of (R)-7-bromo-3-methyl-1,2,3,4-tetrahydrobenzofuro[3,2-c]pyridine (340 mg, 1.28 mmol), 4-(pyridin-2-ylmethoxy)pyridin-2(1H)-one (252 mg, 1.28 mmol), CuI (478 mg, 2.56 mmol), (1S,2S)-N,N′-dimethylcyclohexane-1,2-diamine (357 mg, 2.56 mmol) and Cs₂CO₃ (490 mg, 1.50 mmol) in toluene (7 mL) was degassed for 15 min. The mixture was heated at 105° C. for 24 h with stirring. The reaction mixture was allowed to cool and diluted with a 95:5 (9:1) CH₂Cl₂/(MeOH/NH₄OH) mixture of solvents. The organic layer was washed with NH₄OH and brine (200 mL). The resulting solution was dried over Na₂SO₄, and concentrated under reduced pressure. The residue was purified by flash chromatography on an ISCO 24 g gold column using CH₂Cl₂/CMA[CH₂Cl₂(80%)/MeOH(18%)/NH₄OH(2%)] (0-25% CMA) as eluent. Further purification through column chromatography as well as trituration with ethyl acetate and a 1:1 mixture of CH₂Cl₂/hexanes afforded the title compound as a free base. The free base was converted to the HCl salt using 2 N HCl in Et₂O (0.10 mL, 0.2 mmol). The resulting suspension was concentrated and lyophilized from water and CH₃CN to afford the title compound (67 mg, 24%) as an off-white solid: ¹H NMR (500 MHz, DMSO-d₆) δ 9.66 (br s, 1H), 9.47 (br s, 1H), 8.62 (s, 1H), 7.91 (t, J=7.7 Hz, 1H), 7.68 (m, 2H), 7.61 (d, J=7.6 Hz, 1H), 7.58 (d, J=7.8 Hz, 1H), 7.42 (m, 1H), 7.27 (d, J=8.25 Hz, 1H), 6.17 (d, J=7.6 Hz, 1H), 5.97 (s, 1H), 5.23 (s, 2H), 4.34 (m, 2H), 3.78 (m, 1H), 3.22 (dd, J=17.3, 12.8 Hz, 1H), 2.90 (dd, J=17.3, 7.9 Hz, 1H), 1.46 (d, J=6.5 Hz, 3H); ESI MS m/z 388 [M+H]⁺.

### Example 9

### Preparation of (R)-4-((5-Fluoropyridin-2-yl)methoxy)-1-(3-methyl-1,2,3,4-tetrahydrobenzofuro[3,2-c]pyridin-7-yl)pyridin-2(1H)-one

### a) (R)-tert-Butyl 7-bromo-3-methyl-3,4-dihydrobenzofuro[3,2-c]pyridine-2(1H)-carboxylate

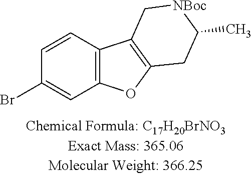 [SMILES: C[C@@H]1Cc2oc3cc(Br)ccc3c2CN1C(=O)OC(C)(C)C]

A solution of (R)-7-bromo-3-methyl-1,2,3,4-tetrahydrobenzofuro[3,2-c]pyridine (880 mg, 3.32 mmol, mixture of regioisomers) in i-PrOH (10 mL) was treated with K₂CO₃ (550 mg, 3.98 mmol) in water (5 mL) and stirred for 5 min. (Boc)₂O (869 mg, 3.98 mmol) was added, and the resulting suspension was stirred at 25° C. overnight. The suspension was diluted with CH₂Cl₂ washed with saturated NaHCO₃ and brine, dried over Na₂SO₄, and concentrated to dryness to afford the title compound (1.41 g, crude) as a light brown semi-solid and as a mixture of regioisomers. This material was used in the next step without further purification: ¹H NMR (500 MHz, CDCl₃) δ 7.59 (m, 1H), 7.34 (m, 2H), 7.28 (m, 1H), 7.08 (m, 1H), 4.92 (m, 1.7H), 4.33 (m, 0.56H), 4.08 (m, 0.77H), 3.11 (m, 1.2), 2.89 (m, 0.6H), 2.68 (m, 0.5H), 2.55 (d, J=16.7 Hz, 1H), 1.52 (s, 9H), 1.45 (d, J=6.6 Hz, 2.4H), 1.19 (d, J=7.04 Hz, 3H).

### b) (R)-4-((5-Fluoropyridin-2-yl)methoxy)-1-(3-methyl-1,2,3,4-tetrahydrobenzofuro[3,2-c]pyridin-7-yl)pyridin-2(1H)-one

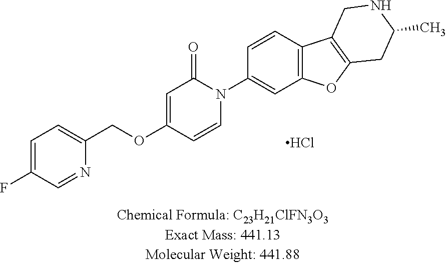 [SMILES: C[C@@H]1Cc2oc3cc(-n4ccc(OCc5ccc(F)cn5)cc4=O)ccc3c2CN1 ; Cl]

A mixture of (R)-tert-butyl 7-bromo-3-methyl-3,4-dihydrobenzofuro[3,2-c]pyridine-2(1H)-carboxylate (404 mg, 1.10 mmol, mixture of regioisomers), 4-[(5-fluoropyridin-2-yl)methoxy]pyridin-2(1H)-one (243 mg, 1.10 mmol), CuI (478 mg, 2.56 mmol), (1S,2S)-N,N′-dimethylcyclohexane-1,2-diamine (357 mg, 2.56 mmol) and Cs₂CO₃ (490 mg, 1.50 mmol) in toluene (7 mL) was degassed for 15 min. The mixture was heated at 105° C. for 24 h with stirring. The reaction mixture was allowed to cool and diluted with a 95:5 (9:1) CH₂Cl₂/(MeOH/NH₄OH) mixture of solvents. The organic layer was washed with NH₄OH and brine (200 mL), dried over Na₂SO₄, and concentrated under reduced pressure. The residue was purified by flash chromatography on an ISCO 24 g gold column using CH₂Cl₂/CMA[CH₂Cl₂(80%)/MeOH (18%)/NH₄OH(2%)](0-20% CMA) as eluent to afford (R)-tert-butyl 7-(4-((5-fluoropyridin-2-yl)methoxy)-2-oxopyridin-1(2H)-yl)-3-methyl-3,4-dihydrobenzofuro[3,2-c]pyridine-2(1H)-carboxylate (316 mg) as a mixture of regiosiomers. The Boc group was removed using 2N HCl in CH₂Cl₂ and worked up with saturated NaHCO₃. The crude material was triturated with ethyl acetate and crystallized from CH₂Cl₂/hexanes (1:1) to afford the title compound as a free base. The free base was converted to the HCl salt using 2 N HCl in Et₂O (0.05 mL, 0.1 mmol). The resulting suspension was concentrated and lyophilized from water and CH₃CN to afford the title compound (47 mg, 10%) as a white solid: ¹H NMR (500 MHz, DMSO-d₆) δ 9.33 (br s, 2H), 8.62 (d, J=2.95 Hz, 1H), 7.82 (t, J=8.7 Hz, 1H), 7.66 (m, 4H), 7.27 (d, J=8.3 Hz, 1H), 6.16 (dd, J=7.6, 4.9 Hz, 1H), 5.99 (s, 1H), 5.21 (s, 2H), 4.41 (m, 2H), 3.78 (m, 1H), 3.22 (dd, J=17.2, 4.6 Hz, 1H), 2.88 (dd, J=17.3, 7.85 Hz, 1H), 1.44 (d, J=6.5 Hz, 3H); ESI MS m/z 406 [M+H]⁺.

### Example 10

### Preparation of 4-((5-Chloropyridin-2-yl)methoxy)-1-(1,2,3,4-tetrahydrobenzofuro[3,2-c]pyridin-7-yl)pyridin-2(1H)-one hydrochloride

### a) 4-((5-Chloropyridin-2-yl)methoxy)pyridin-2(1H)-one

CAS Registry Number 924311-89-7

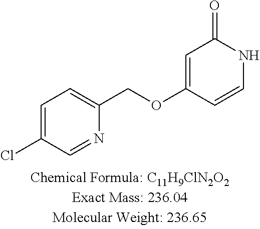 [SMILES: O=c1cc(OCc2ccc(Cl)cn2)cc[nH]1]

This compound was prepared in accordance with the procedure described in PCT Publication No. WO 2009/089482 to Guzzo et al., which is hereby incorporated by reference in its entirety.

### b) tert-Butyl 7-bromo-3,4-dihydrobenzofuro[3,2-c]pyridine-2(1H)-carboxylate

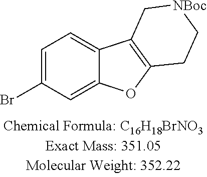 [SMILES: CC(C)(C)OC(=O)N1CCc2oc3cc(Br)ccc3c2C1]

A chilled solution of O-(3-bromophenyl)hydroxylamine hydrochloride (1.12 g, 5 mmol) in AcOH (4.6 mL) was treated with concentrated sulfuric acid (0.46 mL) followed by tert-butyl 4-oxopiperidine-1-carboxylate (1.20 g, 6 mmol) in four portions. The resulting solution was stirred at ambient temperature for 5 min then heated to reflux and held at reflux for 3 h. The resulting solution was cooled in an ice bath and basified with 6 N NaOH. Brine (50 mL) was added, and the resulting mixture was extracted with CH₂Cl₂ (5×60 mL). The combined organic extracts were further washed with brine (100 mL), dried over Na₂SO₄, and concentrated under reduced pressure. The resulting crude solid (1.43 g) was diluted with H₂O (10.3 mL) and iPrOH (7.8 mL). This solution was treated with K₂CO₃ (829 mg, 6 mmol) followed by Boc₂O (1.31 g, 6 mmol), and the resulting solution was stirred at ambient temperature for 18 h. The reaction mixture was diluted with brine (100 mL) and CH₂Cl₂ (100 mL). The resulting layers were separated, and the aqueous phase was extracted with CH₂Cl₂ (3×75 mL). The combined organic extracts were dried over Na₂SO₄ and concentrated under reduced pressure. Purification by flash chromatography (silica gel, (hexanes/EtOAc), 100:0 to 90:10) afforded the title compound (610 mg, 35%) as a white solid: ¹H NMR (500 MHz, CDCl₃) δ 7.59 (s, 1H), 7.35-7.33 (m, 1H), 7.27-7.26 (m, 1H), 4.53 (s, 2H), 3.82 (s, 2H), 2.83 (s, 2H), 1.50 (s, 9H).

### c) tert-Butyl 7-(4-((5-chloropyridin-2-yl)methoxy)-2-oxopyridin-1(2H)-yl)-3,4-dihydrobenzofuro[3,2-c]pyridine-2(1H)-carboxylate

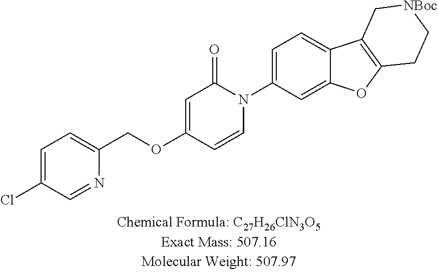 [SMILES: CC(C)(C)OC(=O)N1CCc2oc3cc(-n4ccc(OCc5ccc(Cl)cn5)cc4=O)ccc3c2C1]

A mixture of 4-((5-chloropyridin-2-yl)methoxy)pyridin-2(1H)-one (121 mg, 0.512 mmol), tert-butyl 7-bromo-3,4-dihydrobenzofuro[3,2-c]pyridine-2(1H)-carboxylate (150 mg, 0.43 mmol) and Cs₂CO₃ (180 mg, 0.55 mmol) in toluene (6 mL) in a sealed tube was degassed with a nitrogen stream for 10 min. Trans-N,N′-dimethylcyclohexane-1,2-diamine (91 mg, 0.64 mmol) and CuI (121 mg, 0.635 mmol) were added, and the mixture was degassed for another 2 min. The tube was sealed, and the mixture was heated at 110° C. for 12 h. The mixture was cooled, diluted with 90:9:1 CH₂Cl₂/MeOH/NH₄OH (15 mL) and stirred for 1 h. Then 2:1 brine/NH₄OH (100 mL) was added, and the aqueous phase was extracted with dichloromethane (4×75 mL). The combined organic extracts were washed with 2:1 brine/NH₄OH (3×75 mL), dried over Na₂SO₄, and concentrated under reduced pressure. Purification by flash chromatography (silica gel, (CH₂Cl₂/(90:9:1 CH₂Cl₂/MeOH/NH₄OH), 100:0 to 60:40) afforded the title compound (141 mg, 65%) as white solid: ¹H NMR (500 MHz, DMSO-d₆) δ 8.68-8.66 (m, 1H), 8.02 (dd, J=8.5, 2.5 Hz, 1H), 7.65-7.60 (m, 4H), 7.21 (dd, J=8.5, 2.0 Hz, 1H), 6.15 (dd, J=7.5, 3.0 Hz, 1H), 5.97 (d, J=2.5 Hz, 1H), 5.23 (s, 2H), 4.54 (s, 2H), 3.76 (t, J=5.5 Hz, 2H), 2.87-2.85 (m, 2H), 1.45 (s, 9H).

### d) 4-((5-Chloropyridin-2-yl)methoxy)-1-(1,2,3,4-tetrahydrobenzofuro[3,2-c]pyridin-7-yl)pyridin-2(1H)-one

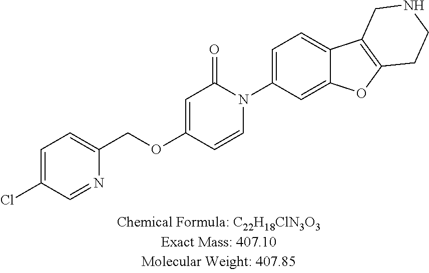 [SMILES: O=c1cc(OCc2ccc(Cl)cn2)ccn1-c1ccc2c3c(oc2c1)CCNC3]

A solution of tert-butyl 7-(4-((5-chloropyridin-2-yl)methoxy)-2-oxopyridin-1(2H)-yl)-3,4-dihydrobenzofuro[3,2-c]pyridine-2(1H)-carboxylate (135 mg, 0.266 mmol) in MeOH (3.0 mL) was treated with 2 N HCl in Et₂O (0.4 mL) and stirred at ambient temperature for 12 h. Additional 2 N HCl in Et₂O (0.2 mL) was added, and the resulting solution was stirred for another 4 h. Saturated aqueous NaHCO₃ (50 mL) was added, and the mixture was extracted with dichloromethane (8×50 mL). The combined organic extracts were dried over Na₂SO₄ and concentrated under reduced pressure. Purification by flash chromatography (silica gel, (CH₂Cl₂/(90:9:1 CH₂Cl₂/MeOH/NH₄OH), 100:0 to 40:60) afforded the title compound (98 mg, 90%) as a white solid: ¹H NMR (500 MHz, DMSO-d₆) δ 8.67-8.66 (m, 1H), 8.02 (dd, J=8.5, 2.0 Hz, 1H), 7.61-7.52 (m, 4H), 7.16 (dd, J=8.5, 2.0 Hz, 1H), 6.14 (dd, J=7.5, 2.5 Hz, 1H), 5.96 (d, J=2.5 Hz, 1H), 5.23 (s, 2H), 3.84 (s, 2H), 3.17 (d, J=5.0 Hz, 1H), 3.08 (t, J=6.0 Hz, 2H), 2.74-2.72 (m, 2H).

### e) 4-((5-Chloropyridin-2-yl)methoxy)-1-(1,2,3,4-tetrahydrobenzofuro[3,2-c]pyridin-7-yl)pyridin-2(1H)-one hydrochloride

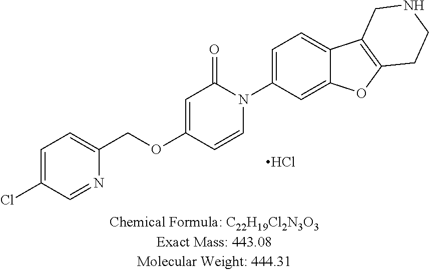 [SMILES: O=c1cc(OCc2ccc(Cl)cn2)ccn1-c1ccc2c3c(oc2c1)CCNC3 ; Cl]

4-((5-Chloropyridin-2-yl)methoxy)-1-(1,2,3,4-tetrahydrobenzofuro[3,2-c]pyridin-7-yl)pyridin-2(1H)-one (95 mg, 0.23 mmol) was suspended in MeOH (3 mL) and treated with 2 N HCl in Et₂O (0.12 mL, 0.23 mmol). The suspension was stirred at ambient temperature for 2 h and concentrated. The residue was lyophilized from acetonitrile-water to provide the title compound (101 mg, 98%) as a white solid: ¹H NMR (500 MHz, DMSO-d₆) δ 9.50 (s, 2H), 8.67 (d, J=2.5 Hz, 1H), 8.03 (dd, J=8.5, 2.5 Hz, 1H), 7.70-7.68 (m, 2H), 7.61 (t, J=7.5 Hz, 2H), 7.27 (dd, J=8.5, 2.0 Hz, 1H), 6.17 (dd, J=7.5, 2.5 Hz, 1H), 5.97 (d, J=3.0 Hz, 1H), 5.24 (s, 2H), 4.36 (s, 2H), 3.55 (d, J=4.0 Hz, 2H), 3.11 (t, J=6.0 Hz, 2H); ESI MS m/z 408 [M+H]⁺; HPLC (Method A)>99% (AUC), t_{R}=12.9 min.

### Example 11

### Preparation of 1′-(1,2,3,4-Tetrahydrobenzofuro[3,2-c]pyridin-7-yl)-5-(trifluoromethyl)-[2,4′-bipyridin]-2′(1′H)-one hydrochloride

### a) 4-(5-(Trifluoromethyl)pyridin-2-yl)pyridin-2(1H)-one

CAS Registry Number 1108184-25-3

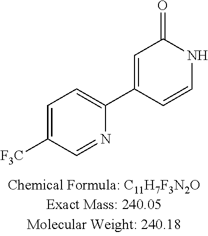 [SMILES: O=c1cc(-c2ccc(C(F)(F)F)cn2)cc[nH]1]

This compound was prepared in accordance with the procedure described in PCT Publication No. WO 2009/089482 to Guzzo et al., which is hereby incorporated by reference in its entirety.

### b) tert-Butyl 7-(2′-oxo-5-(trifluoromethyl)-[2,4′-bipyridin]-1′(2′H)-yl)-3,4-dihydrobenzofuro[3,2-c]pyridine-2(1H)-carboxylate

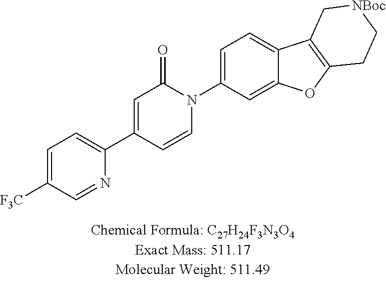 [SMILES: CC(C)(C)OC(=O)N1CCc2oc3cc(-n4ccc(-c5ccc(C(F)(F)F)cn5)cc4=O)ccc3c2C1]

A mixture of 4-(5-(Trifluoromethyl)pyridin-2-yl)pyridin-2(1H)-one (164 mg, 0.683 mmol), tert-butyl 7-bromo-3,4-dihydrobenzofuro[3,2-c]pyridine-2(1H)-carboxylate (200 mg, 0.57 mmol) and Cs₂CO₃ (240 mg, 0.74 mmol) in toluene (7 mL) in a sealed tube was degassed with a nitrogen stream for 10 min. Trans-N,N′-dimethylcyclohexane-1,2-diamine (121 mg, 0.851 mmol) and CuI (162 mg, 0.851 mmol) were added, and the mixture was degassed for another 2 min. The tube was sealed, and the mixture was heated at 110° C. for 12 h. The mixture was cooled, diluted with 90:9:1 CH₂Cl₂/MeOH/NH₄OH (15 mL) and stirred for 1 h. Then 2:1 brine/NH₄OH (100 mL) was added, and the aqueous phase was extracted with dichloromethane (4×75 mL). The combined organic extracts were washed with 2:1 brine/NH₄OH (3×75 mL), dried over Na₂SO₄, and concentrated under reduced pressure. Purification by flash chromatography (silica gel, (CH₂Cl₂/(90:9:1 CH₂Cl₂/MeOH/NH₄OH), 100:0 to 75:25) afforded the title compound (218 mg, 75%) as white solid: ¹H NMR (300 MHz, DMSO-d₆) δ 9.16-9.14 (m, 1H), 8.42-8.34 (m, 2H), 7.87 (d, J=7.2 Hz, 1H), 7.75-7.69 (m, 2H), 7.35-7.30 (m, 2H), 7.08 (dd, J=7.2, 1.8 Hz, 1H), 4.56 (s, 2H), 3.78 (t, J=5.7 Hz, 2H), 2.90-2.86 (m, 2H), 1.46 (s, 9H).

### c) 1′-(1,2,3,4-Tetrahydrobenzofuro[3,2-c]pyridin-7-yl)-5-(trifluoromethyl)-[2,4′-bipyridin]-2′(1′H)-one

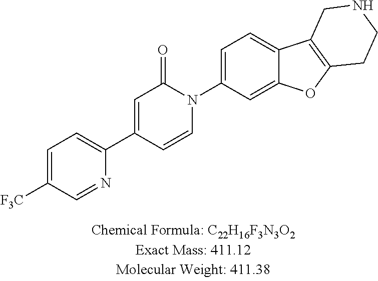 [SMILES: O=c1cc(-c2ccc(C(F)(F)F)cn2)ccn1-c1ccc2c3c(oc2c1)CCNC3]

A solution of tert-butyl 7-(2′-oxo-5-(trifluoromethyl)-[2,4′-bipyridin]-1′(2′H)-yl)-3,4-dihydrobenzofuro[3,2-c]pyridine-2(1H)-carboxylate (210 mg, 0.41 mmol) in MeOH (5.0 mL) was treated with 2 N HCl in Et₂O (0.6 mL) and stirred at ambient temperature for 12 h. Additional 2 N HCl in Et₂O (0.6 mL) was added, and the resulting solution was stirred for another 12 h. Saturated aqueous NaHCO₃ (50 mL) was added, and the mixture was extracted with dichloromethane (8×50 mL). The combined organic extracts were dried over Na₂SO₄ and concentrated under reduced pressure. Purification by flash chromatography (silica gel, (CH₂Cl₂/(90:9:1 CH₂Cl₂/MeOH/NH₄OH), 100:0 to 40:60) afforded the title compound (167 mg, 99%) as a light yellow solid: ¹H NMR (500 MHz, DMSO-d₆) δ 9.15-9.14 (m, 1H), 8.40-8.34 (m, 2H), 7.86 (d, J=7.0 Hz, 1H), 7.69 (d, J=2.0 Hz, 1H), 7.58 (d, J=8.0 Hz, 1H), 7.29-7.28 (m, 2H), 7.07 (dd, J=7.5, 2.5 Hz, 1H), 3.86-3.85 (m, 2H), 3.08 (t, J=5.5 Hz, 2H), 2.74 (t, J=5.5 Hz, 1H), 1.75 (s, 1H).

### d) 1′-(1,2,3,4-Tetrahydrobenzofuro[3,2-c]pyridin-7-yl)-5-(trifluoromethyl)-[2,4′-bipyridin]-2′(1′H)-one hydrochloride

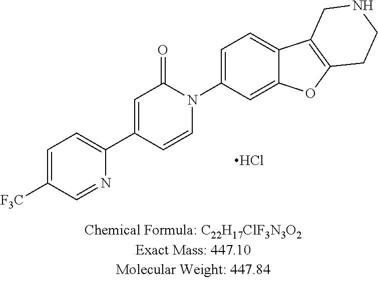 [SMILES: O=c1cc(-c2ccc(C(F)(F)F)cn2)ccn1-c1ccc2c3c(oc2c1)CCNC3 ; Cl]

1′-(1,2,3,4-Tetrahydrobenzofuro[3,2-c]pyridin-7-yl)-5-(trifluoromethyl)-[2,4′-bipyridin]-2′(1′H)-one (167 mg, 0.406 mmol) was suspended in MeOH (4 mL) and treated with 2 N HCl in Et₂O (0.2 mL, 0.4 mmol). The suspension was stirred at ambient temperature for 2 h. The resulting precipitate was filtered, washed with Et₂O and lyophilized from acetonitrile-water to provide the title compound (156 mg, 86%) as a white solid: ¹H NMR (500 MHz, DMSO-d₆) δ 9.54 (s, 2H), 9.15 (t, J=1.0 Hz, 1H), 8.39-8.36 (m, 2H), 7.87 (d, J=7.5 Hz, 1H), 7.82 (d, J=1.5 Hz, 1H), 7.75 (d, J=8.5 Hz, 1H), 7.39 (dd, J=8.5, 2.0 Hz, 1H), 7.30 (d, J=2.0 Hz, 1H), 7.09 (dd, J=7.5, 2.0 Hz, 1H), 4.38 (s, 2H), 3.58-3.56 (m, 2H), 3.15-3.13 (m, 2H); ESI MS m/z 412 [M+H]⁺; HPLC (Method A)>99% (AUC), t_{R}=13.6 min.

### Example 12

### Preparation of 1-(1,2,3,4-Tetrahydrobenzothieno[2,3-c]pyridin-7-yl)-4-(5-(trifluoromethyl)pyridin-2-yl)pyridin-2(1H)-one hydrochloride

### a) tert-Butyl 3-(3-bromophenylthio)-4-oxopiperidine-1-carboxylate

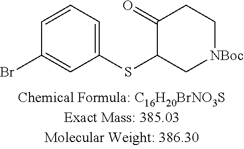 [SMILES: CC(C)(C)OC(=O)N1CCC(=O)C(Sc2cccc(Br)c2)C1]

3-Bromothiophenol (2.30 g, 12.1 mmol) and tert-butyl 4-oxopiperidine-1-carboxylate (2.00 g, 10.1 mmol) were dissolved in CH₂Cl₂ (50 mL) and cooled to 0° C. N-Chlorosuccinimide (1.47 g, 11.1 mmol) was added, and the mixture was stirred for 40 minutes and diluted with water (50 mL). The organic layer was removed, washed with sodium carbonate solution and concentrated. The residue was purified by flash chromatography (silica gel, hexanes/EtOAc, 100:0 to 80:20) to afford the title compound (1.20 g, 31%) as a white foam: ESI MS m/z 330 [M−t-Butyl+H]⁺.

### b) tert-Butyl 7-bromo-3,4-dihydrobenzothieno[2,3-c]pyridine-2(1H)-carboxylate

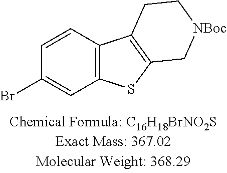 [SMILES: CC(C)(C)OC(=O)N1CCc2c(sc3cc(Br)ccc23)C1]

tert-Butyl 3-(3-bromophenylthio)-4-oxopiperidine-1-carboxylate (1.20 g, 3.11 mmol) was stirred in phosphoric acid (85%, 10 mL) at 130° C. for 3 h. Upon cooling to room temperature, the mixture was diluted with THF (50 mL) and adjusted to pH 10 by the addition of 50% sodium hydroxide solution. Boc₂O (1.01 g, 4.63 mmol) was added, and the mixture was stirred for 2 h and then diluted with CH₂Cl₂ (50 mL). The organic layer was removed, dried with Na₂SO₄, filtered and concentrated. The residue was purified by flash chromatography (silica gel, hexanes/EtOAc, 100:0 to 95:5) to afford the title compound (0.20 g, 18%) as a colorless oil: ¹H NMR (300 MHz, CDCl₃) δ 7.97 (s, 1H), 7.45 (s, 2H), 4.67 (s, 2H), 3.78 (t, J=6.0 Hz, 2H), 2.81 (t, J=6.0 Hz, 2H), 1.50 (s, 9H).

### c) tert-Butyl 7-(2-oxo-4-(5-(trifluoromethyl)pyridin-1(2H)-yl)-3,4-dihydrobenzothieno[2,3-c]pyridine-2(1H)-carboxylate

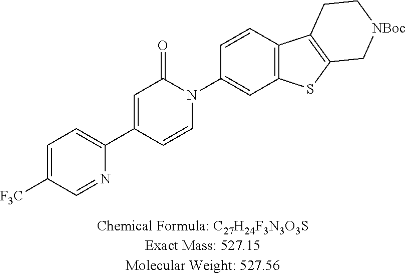 [SMILES: CC(C)(C)OC(=O)N1CCc2c(sc3cc(-n4ccc(-c5ccc(C(F)(F)F)cn5)cc4=O)ccc23)C1]

A mixture of 4-(5-(Trifluoromethyl)pyridin-2-yl)pyridin-2(1H)-one (71 mg, 0.30 mmol), tert-butyl 7-bromo-3,4-dihydrobenzothieno[2,3-c]pyridine-2(1H)-carboxylate (100 mg, 0.271 mmol), CuI (103 mg, 0.543 mmol), trans-N,N′-dimethylcyclohexane-1,2-diamine (77 mg, 0.54 mmol) and Cs₂CO₃ (132 mg, 0.408 mmol) in toluene (10 mL) was degassed with a nitrogen stream for 45 min. The suspension was put under N₂ and stirred at 105° C. for 16 h. The suspension was cooled, 90:9:1 CH₂Cl₂/MeOH/NH₄OH (50 mL) and 2:1 brine/NH₄OH (50 mL) were added, and the resulting mixture was stirred at 25° C. for 30 min. The organic layer was removed, dried over Na₂SO₄, and concentrated under reduced pressure. Purification by flash chromatography (silica gel, (CH₂Cl₂/(90:9:1 CH₂Cl₂/MeOH/NH₄OH), 100:0 to 95:5) afforded the title compound (52 mg, 36%) as a yellow oil: ESI MS m/z 528 [M+H]⁺.

### d) 1-(1,2,3,4-Tetrahydrobenzothieno[2,3-c]pyridin-7-yl)-4-(5-(trifluoromethyl)pyridin-2-yl)pyridin-2(1H)-one hydrochloride

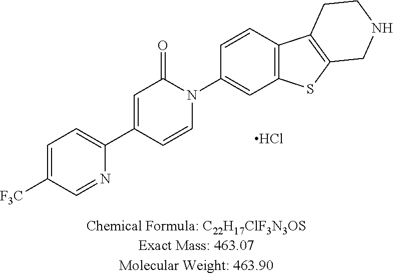 [SMILES: O=c1cc(-c2ccc(C(F)(F)F)cn2)ccn1-c1ccc2c3c(sc2c1)CNCC3 ; Cl]

tert-Butyl 7-(2-oxo-4-(5-(trifluoromethyl)pyridin-2-yl)pyridin-1(2H)-yl)-3,4-dihydrobenzothieno[2,3-c]pyridine-2(1H)-carboxylate (52 mg, 0.099 mmol) was stirred in MeOH (2 mL) and 2 N HCl in Et₂O (10 mL) was added. After stirring for 16 h, the mixture was concentrated and partitioned between saturated Na₂CO₃ solution (10 mL) and CH₂Cl₂ (20 mL). The organic layer was removed, dried over Na₂SO₄ and concentrated under reduced pressure. Purification by flash chromatography (silica gel, (CH₂Cl₂/(90:9:1 CH₂Cl₂/MeOH/NH₄OH), 100:0 to 90:10) afforded the free-base (29 mg, 69%) as a yellow solid. The solid was suspended in MeOH (2 mL), treated with 2 N HCl in Et₂O (0.068 mmol, 34 μL) and concentrated to provide the title compound (31 mg, 100%) as a yellow solid: ¹H NMR (500 MHz, CD₃OD) δ 9.05 (s, 1H), 8.29-8.27 (dd, J=8.4, 2.1 Hz, 1H), 8.22 (d, J=8.4 Hz, 1H), 8.07 (d, J=1.8 Hz, 1H), 7.91 (d, J=8.5 Hz, 1H), 7.83 (d, J=7.0 Hz, 1H), 7.55-7.53 (dd, J=8.5, 1.9 Hz, 1H), 7.39 (d, J=1.5 Hz, 1H), 7.27-7.25 (dd, J=7.2, 2.0 Hz, 1H), 4.59 (s, 2H), 3.69 (t, J=6.5 Hz, 2H), 3.23 (t, J=6.5 Hz, 2H); ESI MS m/z 428 [M+H]⁺; HPLC (Method B) 95.1% (AUC), t_{R}=13.2 min.

### Example 13

### Preparation of 4-((5-Chloropyridin-2-yl)methoxy)-1-(1,2,3,4-tetrahydrobenzothieno[2,3-c]pyridin-7-yl)pyridin-2(1H)-one hydrochloride

### a) tert-Butyl 7-(4-((5-chloropyridin-2-yl)methoxy)-2-oxopyridin-1(2H)-yl)-3,4-dihydrobenzothieno[2,3-c]pyridine-2(1H)-carboxylate

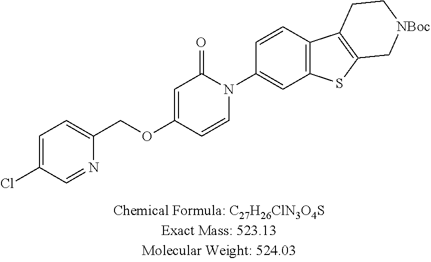 [SMILES: CC(C)(C)OC(=O)N1CCc2c(sc3cc(-n4ccc(OCc5ccc(Cl)cn5)cc4=O)ccc23)C1]

tert-Butyl 7-bromo-3,4-dihydrobenzothieno[2,3-c]pyridine-2(1H)-carboxylate (100 mg, 0.271 mmol) and 4-((5-chloropyridin-2-yl)methoxy)pyridin-2(1H)-one (70 mg, 0.30 mmol) were reacted according to Example 12 (step c) to provide the title compound (52 mg, 36%) as a yellow oil: ESI MS m/z 524 [M+H]⁺.

### b) 4-((5-Chloropyridin-2-yl)methoxy)-1-(1,2,3,4-tetrahydrobenzothieno[2,3-c]pyridin-7

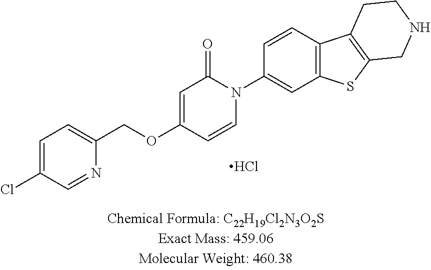 [SMILES: O=c1cc(OCc2ccc(Cl)cn2)ccn1-c1ccc2c3c(sc2c1)CNCC3 ; Cl]

tert-Butyl 7-(4-((5-chloropyridin-2-yl)methoxy)-2-oxopyridin-1(2H)-yl)-3,4-dihydrobenzothieno[2,3-c]pyridine-2(1H)-carboxylate (52 mg, 0.10 mmol) was deprotected and converted to the hydrochloride according to Example 12 (step d) to provide the title compound (32 mg, 70%) as a yellow solid: ¹H NMR (500 MHz, DMSO-d₆) δ 9.66 (s, 2H), 8.67 (d, J=2.4 Hz, 1H), 8.05 (d, J=1.8 Hz, 1H), 8.06-8.02 (dd, J=8.3, 2.5 Hz, 1H), 7.92 (d, J=8.4 Hz, 1H), 7.65 (d, J=8.0 Hz, 1H), 7.61 (d, J=8.0 Hz, 1H), 7.42-7.40 (dd, J=8.5, 1.9 Hz, 1H), 6.18-6.16 (dd, J=7.6, 2.7 Hz, 1H), 5.99 (d, J=2.7 Hz, 1H), 5.23 (s, 2H), 4.49 (s, 2H), 3.52-3.51 (m, 2H), 3.08 (t, J=6.2 Hz, 2H); ESI MS m/z 424 [M+H]⁺; HPLC (Method B) 98.5% (AUC), t_{R}=12.8 min.

### Example 14

### Preparation of 4-((5-Fluoropyridin-2-yl)methoxy)-1-(1,2,3,4-tetrahydrobenzothieno[2,3-c]pyridin-7-yl)pyridin-2(1H)-one hydrochloride

### a) tert-Butyl 7-(4-((5-fluoropyridin-2-yl)methoxy)-2-oxopyridin-1(2H)-yl)-3,4-dihydrobenzothieno[2,3-c]pyridine-2(1H)-carboxylate

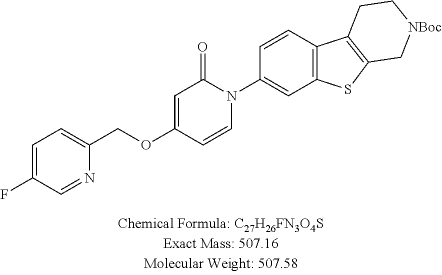 [SMILES: CC(C)(C)OC(=O)N1CCc2c(sc3cc(-n4ccc(OCc5ccc(F)cn5)cc4=O)ccc23)C1]

tert-Butyl 7-bromo-3,4-dihydrobenzothieno[2,3-c]pyridine-2(1H)-carboxylate (207 mg, 0.563 mmol) and 4-((5-fluoropyridin-2-yl)methoxy)pyridin-2(1H)-one (123 mg, 0.563 mmol) were reacted according to Example 12 (step c) to provide the title compound (200 mg, 72%) as a white foam: ESI MS m/z 508 [M+H]⁺.

### b) 4-((5-Fluoropyridin-2-yl)methoxy)-1-(1,2,3,4-tetrahydrobenzothieno[2,3-c]pyridin-7-yl)pyridin-2(1H)-one hydrochloride

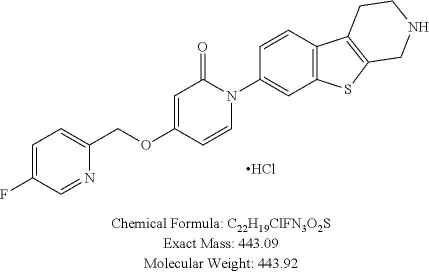 [SMILES: O=c1cc(OCc2ccc(F)cn2)ccn1-c1ccc2c3c(sc2c1)CNCC3 ; Cl]

tert-Butyl 7-(4-((5-fluoropyridin-2-yl)methoxy)-2-oxopyridin-1(2H)-yl)-3,4-dihydrobenzothieno[2,3-c]pyridine-2(1H)-carboxylate (200 mg, 0.394 mmol) was deprotected and converted to the hydrochloride according to Example 12 (step d) to provide the title compound (138 mg, 80%) as a white solid: ¹H NMR (500 MHz, DMSO-d₆) δ 9.84 (s, 2H), 8.62 (d, J=2.9 Hz, 1H), 8.05 (d, J=1.8 Hz, 1H), 7.84-7.80 (m, 2H), 7.67-7.63 (m, 2H), 7.42-7.39 (dd, J=8.5, 1.9 Hz, 1H), 6.17-6.15 (dd, J=7.6, 2.8 Hz, 1H), 6.01 (d, J=2.7 Hz, 1H), 5.22 (s, 2H), 4.50-4.46 (m, 2H), 3.50-3.47 (m, 2H), 3.08 (t, J=6.5 Hz, 2H); ESI MS m/z 408 [M+H]⁺; HPLC (Method B) 99.2% (AUC), t_{R}=9.4 min.

### Example 15

### Preparation of 4-(Benzyloxy)-1-(1,2,3,4-tetrahydrobenzothieno[2,3-c]pyridin-7-yl)pyridin-2(1H)-one

### a) tert-Butyl 7-(4-(benzyloxy)-2-oxopyridin-1(2H)-yl)-3,4-dihydrobenzothieno[2,3-c]pyridine-2(1H)-carboxylate

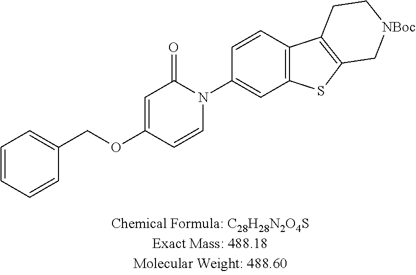 [SMILES: CC(C)(C)OC(=O)N1CCc2c(sc3cc(-n4ccc(OCc5ccccc5)cc4=O)ccc23)C1]

tert-Butyl 7-bromo-3,4-dihydrobenzothieno[2,3-c]pyridine-2(1H)-carboxylate (210 mg, 0.570 mmol) and 4-(benzyloxy)pyridin-2(1H)-one (114 mg, 0.570 mmol) were reacted according to Example 12 (step c) to provide the title compound (150 mg, 54%) as white solid: ESI MS m/z 489 [M+H]⁺.

### b) 4-(Benzyloxy)-1-(1,2,3,4-tetrahydrobenzothieno[2,3-c]pyridin-7-yl)pyridin-2(1H)-one

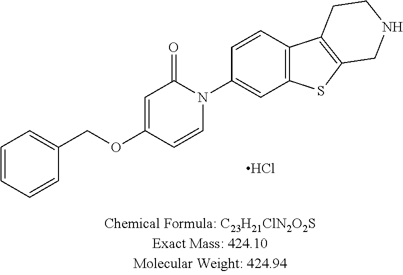 [SMILES: O=c1cc(OCc2ccccc2)ccn1-c1ccc2c3c(sc2c1)CNCC3 ; Cl]

tert-Butyl 7-(4-(benzyloxy)-2-oxopyridin-1(2H)-yl)-3,4-dihydrobenzothieno[2,3-c]pyridine-2(1H)-carboxylate (150 mg, 0.307 mmol) was deprotected and converted to the hydrochloride according to Example 12 (step d) to provide the title compound (130 mg, 100%) as a white solid: ¹H NMR (500 MHz, DMSO-d₆) δ 9.76 (s, 2H), 8.05 (d, J=1.7 Hz, 1H), 7.83 (d, J=8.4 Hz, 1H), 7.62 (d, J=7.6 Hz, 1H), 7.47-7.36 (m, 6H), 6.14-6.12 (dd, J=7.6, 2.6 Hz, 1H), 6.00 (d, J=2.6 Hz, 1H), 5.15 (s, 2H), 4.50-4.43 (m, 2H), 3.53-3.47 (m, 2H), 3.11-3.05 (m, 2H); ESI MS m/z 389 [M+H]⁺; HPLC (Method B)>99% (AUC), t_{R}=12.9 min.

### Example 16

### Preparation of 4-((5-Chloropyridin-2-yl)methoxy)-1-(2,3,4,5-tetrahydro-1H-benzothieno[2,3-c]azepin-8-yl)pyridin-2(1H)-one hydrochloride

### a) tert-Butyl 3-(3-bromophenylthio)-4-oxoazepane-1-carboxylate

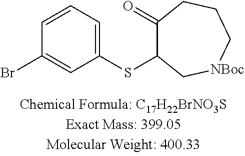 [SMILES: CC(C)(C)OC(=O)N1CCCC(=O)C(Sc2cccc(Br)c2)C1]

3-Bromothiophenol (3.51 g, 18.6 mmol) and tert-butyl 4-oxoazepane-1-carboxylate (3.60 g, 16.9 mmol) were dissolved in CH₂Cl₂ (50 mL) and cooled to 0° C. N-Chlorosuccinimide (1.47 g, 11.1 mmol) was added, and the mixture was stirred for 40 minutes and diluted with water (50 mL). The organic layer was removed, washed with sodium carbonate solution and concentrated. The residue was purified by flash chromatography (silica gel, hexanes/EtOAc, 100:0 to 80:20) and the least polar band, R^{f}0.26 (silica gel, hexanes/EtOAc, 9:1), isolated to afford the title compound (1.00 g, 15%) as a colorless oil: ESI MS m/z 344 [M−t-Butyl+H]⁺. The more polar band, R^{f} 0.20 (silica gel, hexanes/EtOAc, 9:1) isolated to afford tert-butyl 4-(3-bromophenylthio)-5-oxoazepane-1-carboxylate (750 mg, 11%) as a colorless oil: ESI MS m/z 344 [M−t-Butyl+H]⁺.

### b) tert-Butyl 8-bromo-4,5-dihydro-1H-benzothieno[2,3-c]azepine-2(3H)-carboxylate

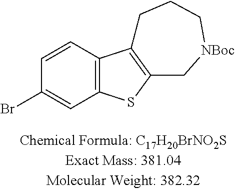 [SMILES: CC(C)(C)OC(=O)N1CCCc2c(sc3cc(Br)ccc23)C1]

tert-Butyl 3-(3-bromophenylthio)-4-oxoazepane-1-carboxylate (1.00 g, 2.50 mmol) was stirred in phosphoric acid (12%, 10 mL) at 130° C. for 3 h. Upon cooling to room temperature, the mixture was diluted with THF (50 mL) and adjusted to pH 10 by the addition of 50% sodium hydroxide solution. Boc₂O (0.54 g, 2.5 mmol) was added, and the mixture was stirred for 2 h and then diluted with CH₂Cl₂ (50 mL). The organic layer was removed, dried with Na₂SO₄, filtered and concentrated. The residue was purified by flash chromatography (silica gel, hexanes/EtOAc, 100:0 to 90:10) to afford the title compound (0.17 g, 18%) as a colorless oil: ¹H NMR (300 MHz, CDCl₃) δ 7.90 (s, 1H), 7.50-7.43 (m, 2H), 4.67-4.51 (br m, 2H), 3.79-3.72 (br m, 2H), 3.00-2.92 (m, 2H), 2.00-1.90 (br m, 2H), 1.40 (s, 9H).

### c) tert-Butyl 8-(4-((5-chloropyridin-2-yl)methoxy)-2-oxopyridin-1(2H)-yl)-4,5-dihydro-1H-benzothieno[2,3-c]azepine-2(3H)-carboxylate

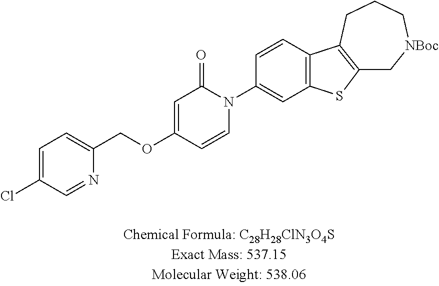 [SMILES: CC(C)(C)OC(=O)N1CCCc2c(sc3cc(-n4ccc(OCc5ccc(Cl)cn5)cc4=O)ccc23)C1]

tert-Butyl 8-bromo-4,5-dihydro-1H-benzothieno[2,3-c]azepine-2(3H)-carboxylate (85 mg, 0.22 mmol) and 4-((5-chloropyridin-2-yl)methoxy)pyridin-2(1H)-one (53 mg, 0.22 mmol) were reacted according to Example 12 (step c) to provide the title compound (90 mg, 76%) as a colorless oil: ESI MS m/z 538 [M+H]⁺.

### d) 4-((5-Chloropyridin-2-yl)methoxy)-1-(2,3,4,5-tetrahydro-1H-benzothieno[2,3-c]azepin-8-yl)pyridin-2(1H)-one hydrochloride

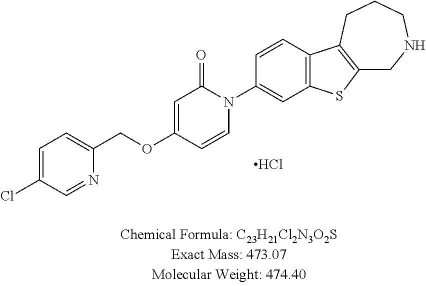 [SMILES: O=c1cc(OCc2ccc(Cl)cn2)ccn1-c1ccc2c3c(sc2c1)CNCCC3 ; Cl]

tert-Butyl 8-(4-((5-chloropyridin-2-yl)methoxy)-2-oxopyridin-1(2H)-yl)-4,5-dihydro-1H-benzothieno[2,3-c]azepine-2(3H)-carboxylate (90 mg, 0.17 mmol) was deprotected and converted to the hydrochloride according to Example 12 (step d) to provide the title compound (64 mg, 79%) as a white solid: ¹H NMR (500 MHz, DMSO-d₆) δ 9.46 (s, 2H), 8.68 (d, J=2.0 Hz, 1H), 8.05-8.01 (m, 2H), 7.92 (d, J=8.6 Hz, 1H), 7.65 (d, J=7.7 Hz, 1H), 7.61 (d, J=8.1 Hz, 1H), 7.42-7.39 (dd, J=8.6, 1.9 Hz, 1H), 6.19-6.16 (dd, J=7.6, 2.7 Hz, 1H), 5.99 (d, J=2.7 Hz, 1H), 5.24 (s, 2H), 4.61-4.52 (m, 2H), 3.54-3.42 (m, 2H), 3.18-3.08 (m, 2H), 2.05-1.95 (m, 2H); ESI MS m/z 438 [M+H]⁺; HPLC (Method B)>99% (AUC), t_{R}=11.5 min.

### Example 17

### Preparation of 1-(2,3,4,5-Tetrahydro-1H-benzothieno[2,3-c]azepin-8-yl)-4-(5-(trifluoromethyl)pyridin-2-yl)pyridin-2(1H)-one

### a) tert-Butyl 8-(2-oxo-4-(5-(trifluoromethyl)pyridin-2-yl)pyridin-1(2H)-yl)-4,5-dihydro-1H-benzothieno[2,3-c]azepine-2(3H)-carboxylate

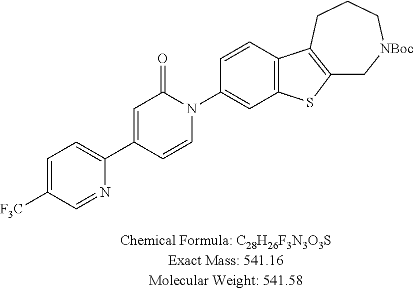 [SMILES: CC(C)(C)OC(=O)N1CCCc2c(sc3cc(-n4ccc(-c5ccc(C(F)(F)F)cn5)cc4=O)ccc23)C1]

tert-Butyl 8-bromo-4,5-dihydro-1H-benzothieno[2,3-c]azepine-2(3H)-carboxylate (85 mg, 0.22 mmol) and 4-(5-(trifluoromethyl)pyridin-2-yl)pyridin-2(1H)-one (52 mg, 0.22 mmol) were reacted according to Example 12 (step c) to provide the title compound (90 mg, 76%) as a brown oil: ESI MS m/z 542 [M+H]⁺.

### b) 1-(2,3,4,5-Tetrahydro-1H-benzothieno[2,3-c]azepin-8-yl)-4-(5-(trifluoromethyl)pyridin-2-ylpyridin-2(1H)-one hydrochloride

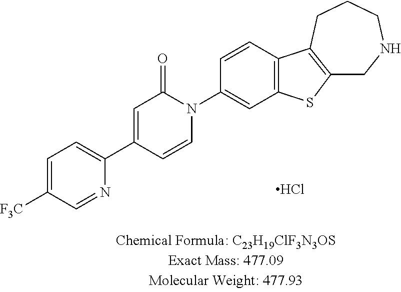 [SMILES: O=c1cc(-c2ccc(C(F)(F)F)cn2)ccn1-c1ccc2c3c(sc2c1)CNCCC3 ; Cl]

tert-Butyl 8-(2-oxo-4-(5-(trifluoromethyl)pyridin-2-yl)pyridin-1(2H)-yl)-4,5-dihydro-1H-benzothieno[2,3-c]azepine-2(3H)-carboxylate (90 mg, 0.16 mmol) was deprotected and converted to the hydrochloride according to Example 12 (step d) to provide the title compound (65 mg, 85%) as a yellow solid: ¹H NMR (500 MHz, DMSO-d₆) δ 9.35 (s, 2H), 9.15 (s, 1H), 8.45-8.31 (m, 2H), 8.17 (d, J=1.8 Hz, 1H), 7.97 (d, J=8.7 Hz, 1H), 7.90 (d, J=7.2 Hz, 1H), 7.54-7.51 (dd, J=8.6, 1.9 Hz, 1H), 7.32 (d, J=1.6 Hz, 1H), 7.12-7.09 (dd, J=7.2, 1.9 Hz, 1H), 4.61 (s, 2H), 3.56-3.45 (m, 2H), 3.22-3.20 (m, 2H), 2.08-1.92 (m, 2H); ESI MS m/z 442 [M+H]⁺; HPLC (Method B)>99% (AUC), t_{R}=14.2 min.

### Example 18

### Preparation of 4-((5-Fluoropyridin-2-yl)methoxy)-1-(2,3,4,5-tetrahydro-1H-benzothieno[2,3-c]azepin-8-yl)pyridin-2(1H)-one

### a) tert-Butyl 8-(4-((5-fluoropyridin-2-yl)methoxy)-2-oxopyridin-1(2H)-yl)-4,5-dihydro-1H-benzothieno[2,3-c]azepine-2(3H)-carboxylate

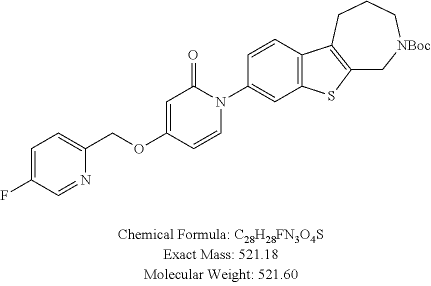 [SMILES: CC(C)(C)OC(=O)N1CCCc2c(sc3cc(-n4ccc(OCc5ccc(F)cn5)cc4=O)ccc23)C1]

tert-Butyl 8-bromo-4,5-dihydro-1H-benzothieno[2,3-c]azepine-2(3H)-carboxylate (70 mg, 0.18 mmol) and 4-((5-fluoropyridin-2-yl)methoxy)pyridin-2(1H)-one (40 mg, 0.18 mmol) were reacted according to Example 12 (step c) to provide the title compound (65 mg, 59%) as a white foam: ESI MS m/z 522 [M+H]⁺.

### b) 4-((5-Fluoropyridin-2-yl)methoxy)-1-(2,3,4,5-tetrahydro-1H-benzothieno[2,3-c]azepin-8-yl)pyridin-2(1H)-one hydrochloride

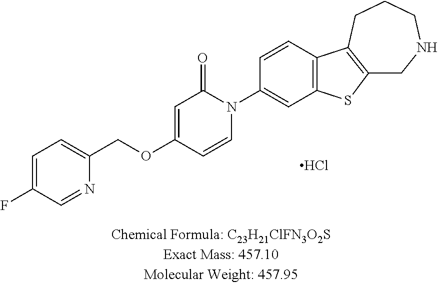 [SMILES: O=c1cc(OCc2ccc(F)cn2)ccn1-c1ccc2c3c(sc2c1)CNCCC3 ; Cl]

tert-Butyl 8-(4-((5-fluoropyridin-2-yl)methoxy)-2-oxopyridin-1(2H)-yl)-4,5-dihydro-1H-benzothieno[2,3-c]azepine-2(3H)-carboxylate (65 mg, 0.12 mmol) was deprotected and converted to the hydrochloride according to Example 12 (step d) to provide the title compound (47 mg, 85%) as a white solid: ¹H NMR (500 MHz, DMSO-d) δ 9.42 (s, 2H), 8.62 (d, J=2.9 Hz, 1H), 8.03 (d, J=1.9 Hz, 1H), 7.91 (d, J=8.6 Hz, 1H), 7.84-7.81 (td, J=8.7, 2.9 Hz, 1H), 7.67-7.63 (m, 2H), 7.41-7.39 (dd, J=8.6, 1.9 Hz, 1H), 6.17-6.15 (dd, J=7.6, 2.7 Hz, 1H), 6.00 (d, J=2.8 Hz, 1H), 5.22 (s, 2H), 4.60-4.56 (m, 2H), 3.51-3.45 (m, 2H), 3.16-3.11 (m, 2H), 2.03-1.97 (m, 2H); ESI MS m/z 422 [M+H]⁺; HPLC (Method B)>99% (AUC), t_{R}=9.9 min.

### Example 19

### Preparation of 4-(Benzyloxy)-1-(2,3,4,5-tetrahydro-1H-benzothieno[2,3-c]azepin-8-yl)pyridin-2(1H)-one

### a) tert-Butyl 8-(4-(benzyloxy)-2-oxopyridin-1(2H)-yl)-4,5-dihydro-1H-benzothieno[2,3-c]azepine-2(3H)-carboxylate

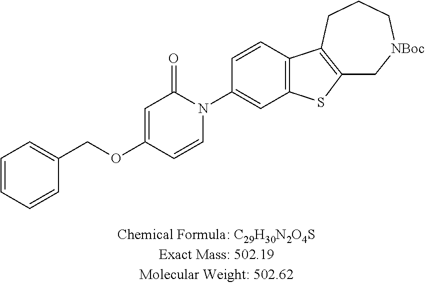 [SMILES: CC(C)(C)OC(=O)N1CCCc2c(sc3cc(-n4ccc(OCc5ccccc5)cc4=O)ccc23)C1]

tert-Butyl 8-bromo-4,5-dihydro-1H-benzothieno[2,3-c]azepine-2(3H)-carboxylate (65 mg, 0.17 mmol) and 4-(benzyloxy)pyridin-2(1H)-one (34 mg, 0.17 mmol) were reacted according to Example 12 (step c) to provide the title compound (85 mg, 100%) as a colorless oil: ESI MS m/z 503 [M+H]⁺.

### b) 4-(Benzyloxy)-1-(2,3,4,5-tetrahydro-1H-benzothieno[2,3-c]azepin-8-yl)pyridin-2(1H)-one hydrochloride

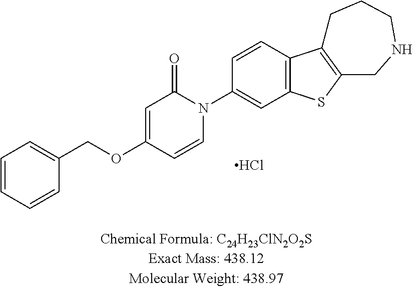 [SMILES: O=c1cc(OCc2ccccc2)ccn1-c1ccc2c3c(sc2c1)CNCCC3 ; Cl]

tert-Butyl 8-(4-(benzyloxy)-2-oxopyridin-1(2H)-yl)-4,5-dihydro-1H-benzothieno[2,3-c]azepine-2(3H)-carboxylate (85 mg, 0.17 mmol) was deprotected and converted to the hydrochloride according to Example 12 (step d) to provide the title compound (64 mg, 85%) as a white solid: ¹H NMR (500 MHz, DMSO-d₆) δ 9.31 (s, 2H), 8.03 (d, J=1.9 Hz, 1H), 7.91 (d, J=8.6 Hz, 1H), 7.62 (d, J=7.6 Hz, 1H), 7.48-7.36 (m, 6H), 6.14-6.12 (dd, J=7.6, 2.7 Hz, 1H), 6.00 (d, J=2.7 Hz, 1H), 5.15 (s, 2H), 4.60-4.56 (m, 2H), 3.51-3.46 (m, 2H), 3.14-3.12 (m, 2H), 2.00-1.97 (m, 2H); ESI MS m/z 403 [M+H]⁺; HPLC (Method B)>99% (AUC), t_{R}=14.0 min.

### Example 20

### Preparation of 4-((5-Chloropyridin-2-yl)methoxy)-1-(2,3,4,5-tetrahydro-1H-benzothieno[2,3-d]azepin-8-yl)pyridin-2(1H)-one

### a) tert-Butyl 8-bromo-4,5-dihydro-1H-benzothieno[2,3-d]azepine-3(2H)-carboxylate

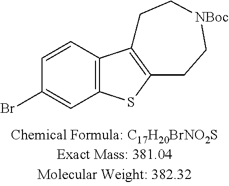 [SMILES: CC(C)(C)OC(=O)N1CCc2sc3cc(Br)ccc3c2CC1]

tert-Butyl 4-(3-bromophenylthio)-5-oxoazepane-1-carboxylate (700 mg, 1.75 mmol), prepared in example 16 (step a), was stirred in phosphoric acid (12%, 10 mL) at 130° C. for 3 h. Upon cooling to room temperature, the mixture was diluted with THF (50 mL) and adjusted to pH 10 by the addition of 50% sodium hydroxide solution. Boc₂O (409 mg, 1.88 mmol) was added, and the mixture was stirred for 2 h and then diluted with CH₂Cl₂ (50 mL). The organic layer was removed, dried with Na₂SO₄, filtered and concentrated. The residue was purified by flash chromatography (silica gel, hexanes/EtOAc, 100:0 to 90:10) to afford the title compound (57 mg, 8.5%) as a colorless oil: ¹H NMR (300 MHz, CDCl₃) δ 7.86 (s, 1H), 7.43 (s, 2H), 3.74-3.65 (m, 4H), 3.11-2.94 (m, 4H), 1.49 (s, 9H).

### b) tert-Butyl 8-(4-((5-chloropyridin-2-yl)methoxy)-2-oxopyridin-1(2H)-yl)-4,5-dihydro-1H-benzothieno[2,3-d]azepine-3(2H)-carboxylate

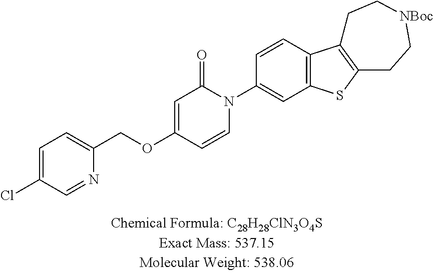 [SMILES: CC(C)(C)OC(=O)N1CCc2sc3cc(-n4ccc(OCc5ccc(Cl)cn5)cc4=O)ccc3c2CC1]

tert-Butyl 8-bromo-4,5-dihydro-1H-benzothieno[2,3-d]azepine-3(2H)-carboxylate (50 mg, 0.13 mmol) and 4-((5-chloropyridin-2-yl)methoxy)pyridin-2(1H)-one (30 mg, 0.13 mmol) were reacted according to Example 12 (step c) to provide the title compound (55 mg, 78%) as a colorless oil: ESI MS m/z 538 [M+H]⁺.

### c) 4-((5-Chloropyridin-2-yl)methoxy)-1-(2,3,4,5-tetrahydro-1H-benzothieno[2,3-d]azepin-8-yl)pyridin-2(1H)-one hydrochloride

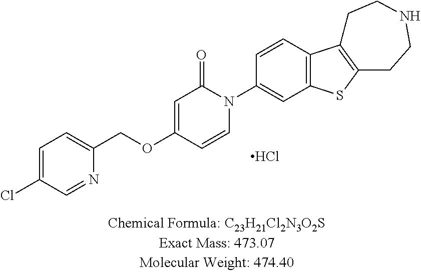 [SMILES: O=c1cc(OCc2ccc(Cl)cn2)ccn1-c1ccc2c3c(sc2c1)CCNCC3 ; Cl]

tert-Butyl 8-(4-((5-chloropyridin-2-yl)methoxy)-2-oxopyridin-1(2H)-yl)-4,5-dihydro-1H-benzothieno[2,3-d]azepine-3(2H)-carboxylate (55 mg, 0.10 mmol) was deprotected and converted to the hydrochloride according to Example 12 (step d) to provide the title compound (39 mg, 82%) as a white solid: ¹H NMR (500 MHz, DMSO-d₆) δ 9.50 (s, 2H), 8.68 (d, J=2.5 Hz, 1H), 8.05-8.01 (dd, J=8.4, 2.5 Hz, 1H), 7.97 (d, J=1.8 Hz, 1H), 7.84 (d, J=8.6 Hz, 1H), 7.66-7.58 (m, 2H), 7.39-7.36 (dd, J=8.6, 2.0 Hz, 1H), 6.18-6.15 (dd, J=7.6, 2.7 Hz, 1H), 5.98 (d, J=2.7 Hz, 1H), 5.23 (s, 2H), 3.43-3.24 (m, 8H); ESI MS m/z 438 [M+H]⁺; HPLC (Method B)>99% (AUC), t_{R}=12.0 min.

### Example 21

### Preparation of 4-((5-Fluoropyridin-2-yl)methoxy)-1-(2,3,4,5-tetrahydro-1H-benzothieno[2,3-d]azepin-8-yl)pyridin-2(1H)-one

### a) tert-Butyl 8-(4-((5-fluoropyridin-2-yl)methoxy)-2-oxopyridin-1(2H)-yl)-4,5-dihydro-1H-benzothieno[2,3-d]azepine-3(2H)-carboxylate

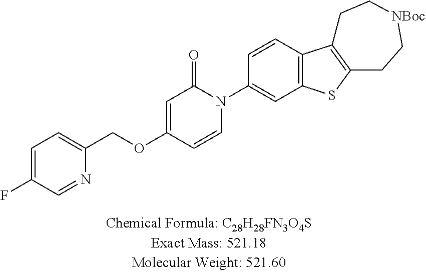 [SMILES: CC(C)(C)OC(=O)N1CCc2sc3cc(-n4ccc(OCc5ccc(F)cn5)cc4=O)ccc3c2CC1]

tert-Butyl 8-bromo-4,5-dihydro-1H-benzothieno[2,3-d]azepine-3(2H)-carboxylate (50 mg, 0.13 mmol) and 4-((5-fluoropyridin-2-yl)methoxy)pyridin-2(1H)-one (30 mg, 0.13 mmol) were reacted according to Example 12 (step c) to provide the title compound (47 mg, 70%) as a colorless oil: ESI MS m/z 522 [M+H]⁺.

### b) 4-((5-Fluoropyridin-2-yl)methoxy)-1-(2,3,4,5-tetrahydro-1H-benzothieno[2,3-d]azepin-8-yl)pyridin-2(1H)-one hydrochloride

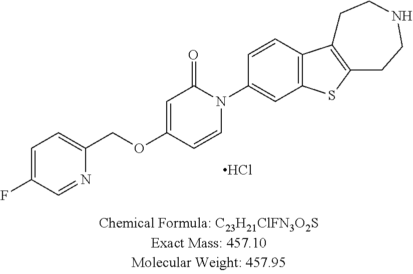 [SMILES: O=c1cc(OCc2ccc(F)cn2)ccn1-c1ccc2c3c(sc2c1)CCNCC3 ; Cl]

tert-Butyl 8-(4-((5-fluoropyridin-2-yl)methoxy)-2-oxopyridin-1(2H)-yl)-4,5-dihydro-1H-benzothieno[2,3-d]azepine-3(2H)-carboxylate (47 mg, 0.090 mmol) was deprotected and converted to the hydrochloride according to Example 12 (step d) to provide the title compound (38 mg, 39%) as a white solid: ¹H NMR (500 MHz, DMSO-d₆) δ 9.39 (s, 2H), 8.62 (d, J=2.9 Hz, 1H), 7.97 (d, J=1.9 Hz, 1H), 7.86-7.79 (m, 2H), 7.68-7.61 (m, 2H), 7.38-7.25 (dd, J=8.5, 1.9 Hz, 1H), 6.17-6.14 (dd, J=7.6, 2.7 Hz, 1H), 5.99 (d, J=2.6 Hz, 1H), 5.22 (s, 2H), 3.39-3.24 (m, 8H); ESI MS m/z 422 [M+H]⁺; HPLC (Method B)>99% (AUC), t_{R}=10.3 min.

### Example 22

### Preparation of 4-(Benzyloxy)-1-(2,3,4,5-tetrahydro-1H-benzothieno[2,3-d]azepin-8-yl)pyridin-2(1H)-one

### a) tert-Butyl 8-(4-(benzyloxy)-2-oxopyridin-1(2H)-yl)-4,5-dihydro-1H-benzothieno[2,3-d]azepine-3(2H)-carboxylate

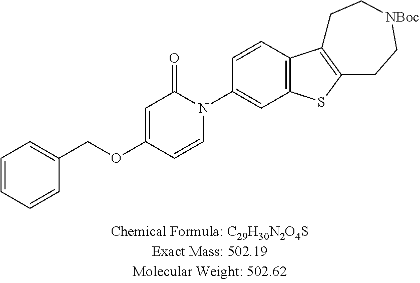 [SMILES: CC(C)(C)OC(=O)N1CCc2sc3cc(-n4ccc(OCc5ccccc5)cc4=O)ccc3c2CC1]

tert-Butyl 8-bromo-4,5-dihydro-1H-benzothieno[2,3-d]azepine-3(2H)-carboxylate (50 mg, 0.13 mmol) and 4-(benzyloxy)pyridin-2(1H)-one (26 mg, 0.13 mmol) were reacted according to Example 12 (step c) to provide the title compound (25 mg, 38%) as a colorless oil: ESI MS m/z 503 [M+H]⁺.

### b) 4-(Benzyloxy)-1-(2,3,4,5-tetrahydro-1H-benzothieno[2,3-d]azepin-8-yl)pyridin-2(1H)-one hydrochloride

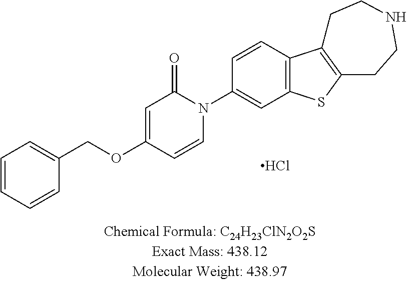 [SMILES: O=c1cc(OCc2ccccc2)ccn1-c1ccc2c3c(sc2c1)CCNCC3 ; Cl]

tert-Butyl 8-(4-(benzyloxy)-2-oxopyridin-1(2H)-yl)-4,5-dihydro-1H-benzothieno[2,3-d]azepine-3(2H)-carboxylate (25 mg, 0.050 mmol) was deprotected and converted to the hydrochloride according to Example 12 (step d) to provide the title compound (18 mg, 82%) as a white solid: ¹H NMR (500 MHz, DMSO-d₆) δ 9.43 (s, 2H), 7.97 (d, J=1.9 Hz, 1H), 7.82 (d, J=8.0 Hz, 1H), 7.61 (d, J=7.6 Hz, 1H), 7.50-7.33 (m, 6H), 6.14-6.11 (dd, J=7.6, 2.7 Hz, 1H), 5.99 (d, J=2.6 Hz, 1H), 5.15 (s, 2H), 3.40-3.22 (m, 8H); ESI MS m/z 403 [M+H]⁺; HPLC (Method B)>99% (AUC), t_{R}=14.8 min.

### Example 23

### Preparation of 4-((5-Fluoropyridin-2-yl)methoxy)-1-(2-methyl-1,2,3,4-tetrahydrobenzofuro[3,2-c]pyridin-7-yl)pyridin-2(1H)-one hydrochloride

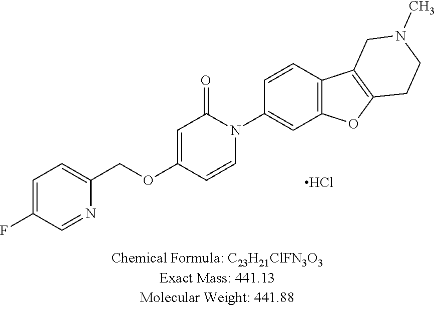 [SMILES: CN1CCc2oc3cc(-n4ccc(OCc5ccc(F)cn5)cc4=O)ccc3c2C1 ; Cl]

4-((5-Fluoropyridin-2-yl)methoxy)-1-(1,2,3,4-tetrahydrobenzofuro[3,2-c]pyridin-7-yl)pyridin-2(1H)-one (107 mg, 0.273 mmol) was reacted according to Example 58 to provide the title compound (44 mg, 36%) as an off-white solid: ¹H NMR (300 MHz, CD₃OD) δ 8.59 (dt, J=2.8, 0.8 Hz, 1H), 7.90-7.60 (m, 5H), 7.31 (dd, J=8.3, 1.9 Hz, 1H), 6.40 (dd, J=7.6, 2.7 Hz, 1H), 6.19 (d, J=2.7 Hz, 1H), 5.32 (s, 2H), 4.75 (d, J=14.9 Hz, 1H), 4.41 (d, J=15.0 Hz, 1H), 3.91-3.89 (m, 1H), 3.76-3.60 (m, 1H), 3.30-3.20 (m, 2H), 3.16 (s, 3H); ESI MS m/z 406 [M+H]⁺; HPLC (Method A)>98.8% (AUC), t_{R}=12.3 min.

### Example 24

### Preparation of 1-(2-Ethyl-1,2,3,4-tetrahydrobenzofuro[3,2-c]pyridin-7-yl)-4-((5-fluoropyridin-2-yl)methoxy)pyridin-2(1H)-one

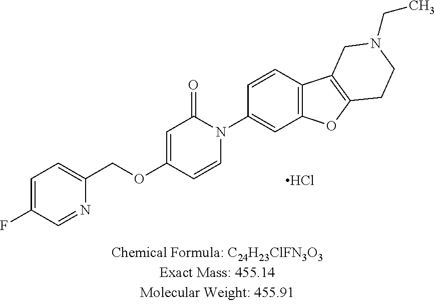 [SMILES: CCN1CCc2oc3cc(-n4ccc(OCc5ccc(F)cn5)cc4=O)ccc3c2C1 ; Cl]

4-((5-Fluoropyridin-2-yl)methoxy)-1-(1,2,3,4-tetrahydrobenzofuro[3,2-c]pyridin-7-yl)pyridin-2(1H)-one (44 mg, 0.11 mmol) was reacted according to Example 56 to provide the title compound (35 mg, 70%) as an off-white solid: ¹H NMR (300 MHz, CD₃OD) δ 8.65-8.57 (m, 1H), 7.92-7.60 (m, 5H), 7.36-7.26 (m, 1H), 6.42 (dd, J=7.6, 2.7 Hz, 1H), 6.20 (d, J=2.8 Hz, 1H), 5.33 (s, 2H), 4.88-4.72 (m, 1H), 4.39 (d, J=15.3 Hz, 1H), 4.02-3.92 (m, 1H), 3.64-3.62 (m, 1H), 3.56-3.43 (m, 2H), 3.30-3.24 (m, 2H), 1.56-1.50 (t, J=7.3 Hz, 3H); ESI MS m/z 420 [M+H]⁺; HPLC (Method A)>95.8% (AUC), t_{R}=12.5 min.

### Example 25

### Preparation of 1-(2-Acetyl-1,2,3,4-tetrahydrobenzofuro[3,2-c]pyridin-7-yl)-4-(benzyloxy)pyridin-2(1H)-one

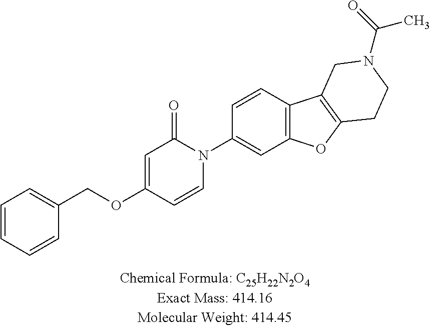 [SMILES: CC(=O)N1CCc2oc3cc(-n4ccc(OCc5ccccc5)cc4=O)ccc3c2C1]

4-(Benzyloxy)-1-(1,2,3,4-tetrahydrobenzofuro[3,2-c]pyridin-7-yl)pyridin-2(1H)-one (100 mg, 0.256 mmol) was reacted according to Example 53 to provide the title compound (87 mg, 100%) as an off-white solid and as a mixture of rotamers: ¹H NMR (300 MHz, CDCl₃) δ 7.50-7.34 (m, 6H), 7.29-7.16 (m, 3H), 6.09-6.05 (m, 2H), 5.05 (s, 2H), 4.74 (d, J=2.1 Hz, 1H), 4.60 (t, J=2.0 Hz, 1H), 4.02 (t, J=5.8 Hz, 1H), 3.84 (t, J=5.7 Hz, 1H), 3.01-2.83 (m, 2H), 2.24 (s, 1.5H), 2.23 (s, 1.5H); ESI MS m/z 415 [M+H]⁺; HPLC (Method A)>99% (AUC), t_{R}=18.3 min.

### Example 26

### Preparation of 4-(Benzyloxy)-1-(2-ethyl-1,2,3,4-tetrahydrobenzofuro[3,2-c]pyridin-7-yl)pyridin-2(1H)-one

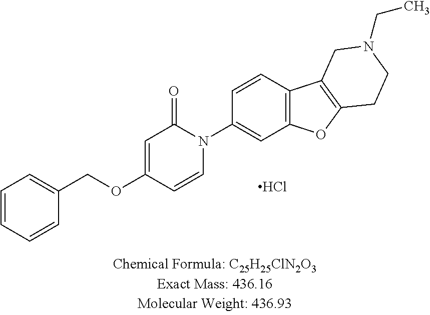 [SMILES: CCN1CCc2oc3cc(-n4ccc(OCc5ccccc5)cc4=O)ccc3c2C1 ; Cl]

4-(Benzyloxy)-1-(1,2,3,4-tetrahydrobenzofuro[3,2-c]pyridin-7-yl)pyridin-2(1H)-one (110 mg, 0.295 mmol) was reacted according to Example 56 to provide the title compound (111 mg, 86%) as an off-white solid: ¹H NMR (300 MHz, CD₃OD) δ 7.74-7.59 (m, 3H), 7.52-7.26 (m, 6H), 6.38 (dd, J=7.6, 2.7 Hz, 1H), 6.18 (d, J=2.7 Hz, 1H), 5.21 (s, 2H), 4.77 (d, J=15.0 Hz, 1H) 4.39 (d, J=15.0 Hz, 1H), 4.00-3.90 (m, 1H), 3.72-3.56 (m, 1H), 3.53-3.41 (m, 2H), 3.30-3.25 (m, 2H), 1.50 (t, J=7.3 Hz, 3H); ESI MS m/z 401 [M+H]⁺; HPLC (Method A) 98.1% (AUC), t_{R}=14.0 min.

### Example 27

### Preparation of 4-(Benzyloxy)-1-(2-methyl-1,2,3,4-tetrahydrobenzofuro[3,2-c]pyridin-7-yl)pyridin-2(1H)-one hydrochloride

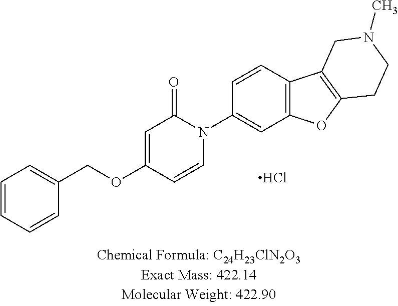 [SMILES: CN1CCc2oc3cc(-n4ccc(OCc5ccccc5)cc4=O)ccc3c2C1 ; Cl]

4-(Benzyloxy)-1-(1,2,3,4-tetrahydrobenzofuro[3,2-c]pyridin-7-yl)pyridin-2(1H)-one (100 mg, 0.269 mmol) was reacted according to Example 58 to provide the title compound (110 mg, 89%) as an off-white solid: ¹H NMR (300 MHz, CD₃OD) δ 7.73-7.62 (m, 3H), 7.52-7.27 (m, 6H), 6.44 (dd, J=7.6, 2.7 Hz, 1H), 6.23 (d, J=2.7 Hz, 1H), 5.23 (s, 2H), 4.75 (d, J=14.8 Hz, 1H), 4.48-4.35 (m, 1H), 3.97-3.88 (m, 1H), 3.76-3.60 (m, 1H), 3.30-3.25 (m, 2H), 3.16 (s, 3H); ESI MS m/z 387 [M+H]⁺; HPLC (Method B)>99% (AUC), t_{R}=13.7 min.

### Example 28

### Preparation of 4-((5-Fluoropyridin-2-yl)methoxy)-1-(2-isopropyl-1,2,3,4-tetrahydrobenzofuro[3,2-c]pyridin-7-yl)pyridin-2(1H-one hydrochloride

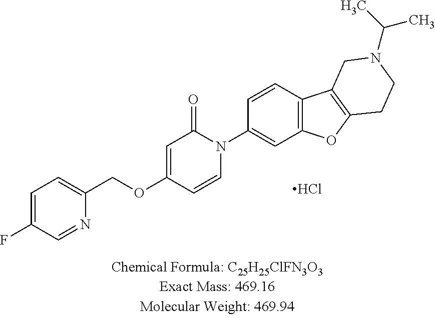 [SMILES: CC(C)N1CCc2oc3cc(-n4ccc(OCc5ccc(F)cn5)cc4=O)ccc3c2C1 ; Cl]

4-((5-Fluoropyridin-2-yl)methoxy)-1-(1,2,3,4-tetrahydrobenzofuro[3,2-c]pyridin-7-yl)pyridin-2(1H)-one (100 mg, 0.256 mmol) was reacted according to Example 57 to provide the title compound (94 mg, 78%) as a white foam: ¹H NMR (300 MHz, CD₃OD) δ 8.56 (dt, J=2.9, 0.8 Hz, 1H), 7.86-7.59 (m, 5H), 7.31 (dd, J=8.3, 1.8 Hz, 1H), 6.38 (dd, J=7.6, 2.7 Hz, 1H), 6.17 (d, J=2.7 Hz, 1H), 5.30 (s, 2H), 4.69-4.46 (m, 2H), 3.97-3.80 (m, 2H), 3.65 (td, J=11.5, 5.6 Hz, 1H), 3.30-3.20 (m, 2H), 1.55-1.50 (m, 6H); ESI MS m/z 434 [M+H]⁺; HPLC (Method A)>99% (AUC), t_{R}=12.9 min.

### Example 29

### Preparation of 1-(2-Acetyl-1,2,3,4-tetrahydrobenzofuro[3,2-c]pyridin-7-yl)-4-((5-fluoropyridin-2-yl)methoxy)pyridin-2(1H)-one

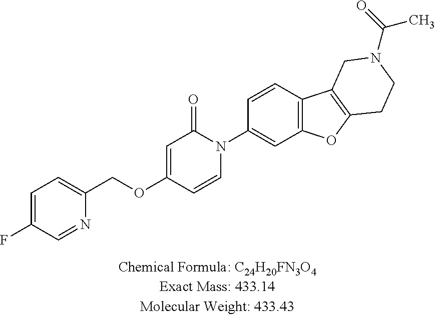 [SMILES: CC(=O)N1CCc2oc3cc(-n4ccc(OCc5ccc(F)cn5)cc4=O)ccc3c2C1]

4-((5-Fluoropyridin-2-yl)methoxy)-1-(1,2,3,4-tetrahydrobenzofuro[3,2-c]pyridin-7-yl)pyridin-2(1H)-one (100 mg, 0.256 mmol) was reacted according to Example 53 to provide the title compound (110 mg, 100%) as a white solid and as a mixture of rotamers: ¹H NMR (300 MHz, CDCl₃) δ 8.53-8.45 (m, 1H), 7.56-7.40 (m, 4H), 7.37-7.14 (m, 2H), 6.16-5.99 (m, 2H), 5.17 (2×s, 2H), 4.79-4.71 (m, 1.2H), 4.61 (t, J=2.0 Hz, 0.8H), 4.03 (t, J=5.8 Hz, 0.8H), 3.85 (t, J=5.7 Hz, 1.2H), 3.01-2.83 (m, 2H), 2.24 (m, 3H); ESI MS m/z 434 [M+H]⁺; HPLC (Method A)>99% (AUC), t_{R}=15.9 min.

### Example 30

### Preparation of 4-(Benzyloxy)-1-(2-isopropyl-1,2,3,4-tetrahydrobenzofuro[3,2-c]pyridin-7-yl)pyridin-2(1H)-one hydrochloride

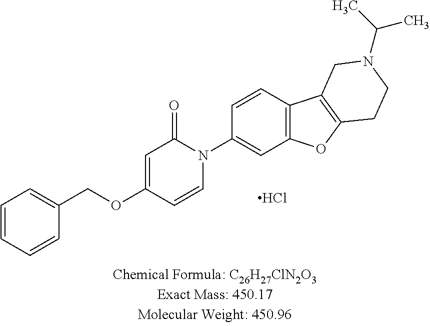 [SMILES: CC(C)N1CCc2oc3cc(-n4ccc(OCc5ccccc5)cc4=O)ccc3c2C1 ; Cl]

4-(Benzyloxy)-1-(1,2,3,4-tetrahydrobenzofuro[3,2-c]pyridin-7-yl)pyridin-2(1H)-one (114 mg, 0.306 mmol) was reacted according to Example 57 to provide the title compound (128 mg, 92%) as a white foam: ¹H NMR (300 MHz, CD₃OD) δ 7.77-7.62 (m, 3H), 7.53-7.28 (m, 6H), 6.47 (dd, J=7.5, 2.6 Hz, 1H), 6.25 (d, J=2.7 Hz, 1H), 5.24 (s, 2H), 4.63 (d, J=14.7 Hz, 1H), 4.52 (d, J=14.8 Hz, 1H), 3.96-3.81 (m, 2H), 3.70-3.60 (m, 1H) 3.30-3.24 (m, 2H), 1.52 (m, 6H); ESI MS m/z 415 [M+H]⁺; HPLC (Method A)>99% (AUC), t_{R}=14.3 min.

### Example 31

### Preparation of 1-(1,2,3,4-Tetrahydrobenzofuro[3,2-c]pyridin-7-yl)-4-((6-(trifluoromethyl)pyridin-2-yl)methoxy)pyridin-2(1H)-one hydrochloride

### a) 2-Chloro-4-((6-(trifluoromethyl)pyridin-2-yl)methoxy)pyridine

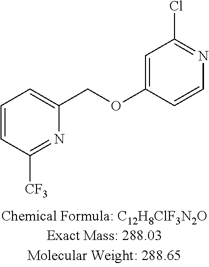 [SMILES: FC(F)(F)c1cccc(COc2ccnc(Cl)c2)n1]

6-(Trifluoromethyl)-2-pyridinemethanol (2.20 g, 12.8 mmol) was reacted according to Example 113 (step c) to provide the title compound (3.30 g, 89%) as a white foam: ESI MS m/z 289 [M+H]⁺.

### b) 4-((6-(Trifluoromethyl)pyridin-2-yl)methoxy)pyridin-2(1H)-one

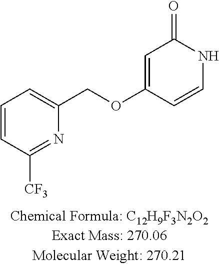 [SMILES: O=c1cc(OCc2cccc(C(F)(F)F)n2)cc[nH]1]

2-Chloro-4-((6-(trifluoromethyl)pyridin-2-yl)methoxy)pyridine (3.30 g, 11.4 mmol) was reacted according to Example 113 (step d) to provide the title compound (2.35 g, 76%) as a white solid: MS m/z 271 [M+H]⁺.

### c) tert-Butyl 7-(2-oxo-4-((6-(trifluoromethyl)pyridin-1(2H)-yl)-3,4-dihydrobenzofuro[3,2-c]pyridine-2(1H)-carboxylate

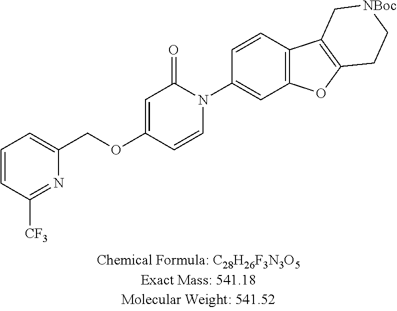 [SMILES: CC(C)(C)OC(=O)N1CCc2oc3cc(-n4ccc(OCc5cccc(C(F)(F)F)n5)cc4=O)ccc3c2C1]

tert-Butyl 7-bromo-3,4-dihydrobenzofuro[3,2-c]pyridine-2(1H)-carboxylate (100 mg, 0.284 mmol) and 4-((6-(trifluoromethyl)pyridin-2-yl)methoxy)pyridin-2(1H)-one (98 mg, 0.36 mmol) were reacted according to Example 12 (step c) to provide the title compound (50 mg, 32%) as a white foam: ESI MS m/z 542 [M+H]⁺.

### d) 1-(1,2,3,4-Tetrahydrobenzofuro[3,2-c]pyridin-7-yl)-4-((6-(trifluoromethyl)pyridin-2-yl)methoxy)pyridin-2(1H)-one hydrochloride

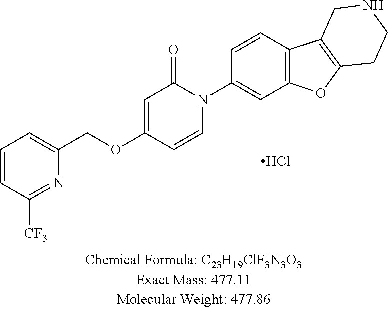 [SMILES: O=c1cc(OCc2cccc(C(F)(F)F)n2)ccn1-c1ccc2c3c(oc2c1)CCNC3 ; Cl]

tert-Butyl 7-(2-oxo-4-((6-(trifluoromethyl)pyridin-2-yl)methoxy)pyridin-1(2H)-yl)-3,4-dihydrobenzofuro[3,2-c]pyridine-2(1H)-carboxylate (50 mg, 0.092 mmol) was deprotected and converted to the hydrochloride according to Example 12 (step d) to provide the title compound (35 mg, 80%) as an off-white solid: ¹H NMR (300 MHz, CD₃OD) δ 8.15 (t, J=7.8 Hz, 1H), 8.00 (d, J=7.5 Hz, 1H), 7.90 (d, J=8.1 Hz, 1H), 7.83 (d, J=7.9 Hz, 1H), 7.79-7.68 (m, 2H), 7.39 (dd, J=8.3, 1.9 Hz, 1H), 6.85 (dd, J=7.5, 2.6 Hz, 1H), 6.55 (d, J=2.6 Hz, 1H), 5.50 (s, 2H), 4.50 (d, J=1.7 Hz, 2H), 3.72 (t, J=6.1 Hz, 2H), 3.31-3.18 (m, 2H); ESI MS m/z 442 [M+H]⁺; HPLC (Method B) 95.8% (AUC), t_{R}=13.9 min.

### Example 32

### Preparation of (S)-4-(Benzyloxy)-1-(3-methyl-1,2,3,4-tetrahydrobenzofuro[3,2-c]pyridin-7-yl)pyridin-2(1H)-one hydrochloride

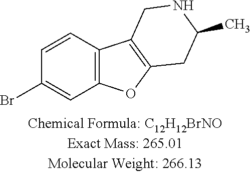

A mixture of O-(3-bromophenyl)hydroxylamine hydrochloride (1.04 g, 4.63 mmol) and (S)-2-methylpiperidin-4-one acetic acid salt (1.57 g, 4.77 mmol) in acetic acid (10 mL) was treated with sulfuric acid (0.5 mL). This mixture was heated at 90° C. for overnight. After cooling, the reaction mixture was quenched with saturated NaHCO₃ solution, extracted with CH₂Cl₂ (250 mL), washed with brine (250 mL), dried (Na₂SO₄) and concentrated to afford a light brown solid (1.3 g as crude material), which was used in the next step without further purification: ESI MS m/z 266 [M+H]⁺.

### b) (S)-4-(Benzyloxy)-1-(3-methyl-1,2,3,4-tetrahydrobenzofuro[3,2-c]pyridin-7-yl)pyridin-2(1H)-one hydrochloride

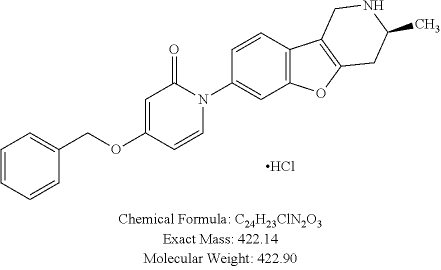 [SMILES: C[C@H]1Cc2oc3cc(-n4ccc(OCc5ccccc5)cc4=O)ccc3c2CN1 ; Cl]

A mixture of (S)-7-bromo-3-methyl-1,2,3,4-tetrahydrobenzofuro[3,2-c]pyridine (300 mg, 1.13 mmol), 4-(benzyloxy)pyridin-2(1H)-one (227 mg, 1.13 mmol), CuI (421 mg, 2.21 mmol), (1S,2S)-N,N′-dimethylcyclohexane-1,2-diamine (315 mg, 2.21 mmol) and Cs₂CO₃ (432 mg, 1.32 mmol) in toluene (7 mL) was degassed for 15 min. This mixture was heated at 105° C. for 24 h with stirring. After cooling, the reaction mixture was diluted with a 95:5 (9:1) CH₂Cl₂/(MeOH/NH₄OH) mixture of solvents. The organic layer was washed with NH₄OH and brine (200 mL), and the resulting solution was dried over Na₂SO₄ and concentrated under reduced pressure. The residue was purified by flash chromatography on an ISCO 40 g gold column using CH₂Cl₂/CMA [CH₂Cl₂ (80%)/MeOH (18%)/NH₄OH (2%)](0-20% CMA) as the eluent to afford the free base of the title compound with reasonable purity. Further purification through column chromatography using CH₂Cl₂/MeOH (85:15) as the eluent afforded pure material which was converted to the hydrochloride using 2 N HCl in Et₂O (0.050 mL, 0.1 mmol). The resulting suspension was concentrated and lyophilized using water and CH₃CN to afford the title compound (24.5 mg, 5%) as an off-white solid: ¹H NMR (500 MHz, DMSO-d₆) δ 9.57 (br s, 1H), 9.39 (br s, 1H), 7.68 (m, 2H), 7.59 (d, J=7.65 Hz, 1H), 7.42 (m, 5H), 7.26 (dd, J=8.2 Hz, 6.6 Hz, 1H), 6.12 (dd, J=7.6 Hz, 5.0 Hz, 1H), 5.98 (s, 1H), 5.15 (s, 2H), 4.42 (m, 2H), 3.79 (br s, 1H), 3.22 (dd, J=17.3 Hz, 13.2 Hz, 1H), 2.90 (dd, J=17.2 Hz, 7.9 Hz, 1H), 1.45 (d, J=6.5 Hz, 3H); ESI MS m/z 387 [M+H]⁺.

### Example 33

### Preparation of (S)-4-(Benzyloxy)-1-(1-methyl-1,2,3,4-tetrahydrobenzofuro[3,2-c]pyridin-7-yl)pyridin-2(1H)-one hydrochloride

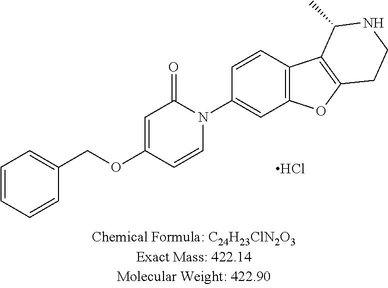 [SMILES: C[C@@H]1NCCc2oc3cc(-n4ccc(OCc5ccccc5)cc4=O)ccc3c21 ; Cl]

The free base of the title compound was obtained as a minor product in Example 32. The hydrochloride was prepared using 2 N HCl in Et₂O (0.025 mL, 0.05 mmol). The resulting suspension was concentrated and lyophilized using water and CH₃CN to afford the title compound (12.7 mg, 2.6%) as an off-white solid: ¹H NMR (500 MHz, DMSO-d₆) δ 9.65 (br s, 1H), 9.24 (br s, 1H), 7.74 (d, J=8.3 Hz, 1H), 7.68 (s, 1H), 7.59 (d, J=7.6 Hz, 1H), 7.43 (m, 5H), 7.26 (dd, J=8.3 Hz, 6.6 Hz, 1H), 6.12 (dd, J=7.6 Hz, 5.0 Hz, 1H), 5.98 (s, 1H), 5.15 (s, 2H), 4.80 (bs, 1H), 3.64 (m, 1H), 3.47 (m, 1H), 3.11 (m, 2H), 1.68 (d, J=6.7 Hz, 3H); ESI MS m/z 387 [M+H]⁺.

### Example 34

### Preparation of (S)-1-(3-Methyl-1,2,3,4-tetrahydrobenzofuro[3,2-c]pyridin-7-yl)-4-(pyridin-2-ylmethoxy)pyridin-2(1H)-one hydrochloride

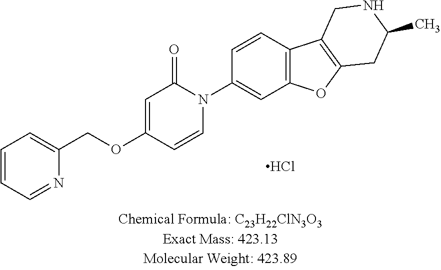 [SMILES: C[C@H]1Cc2oc3cc(-n4ccc(OCc5ccccn5)cc4=O)ccc3c2CN1 ; Cl]

A mixture of (S)-7-bromo-3-methyl-1,2,3,4-tetrahydrobenzofuro[3,2-c]pyridine (300 mg, 1.13 mmol), 4-(pyridin-2-ylmethoxy)pyridin-2(1H)-one (227 mg, 1.13 mmol), CuI (421 mg, 2.21 mmol), (1S,2S)-N,N′-dimethylcyclohexane-1,2-diamine (315 mg, 2.21 mmol) and Cs₂CO₃ (432 mg, 1.32 mmol) in toluene (7 mL) was degassed for 15 min. This mixture was heated at 105° C. for 24 h with stirring. After cooling, the reaction mixture was diluted with a 95:5 (9:1) CH₂Cl₂/(MeOH/NH₄OH) mixture of solvents. The organic layer was washed with NH₄OH and brine (200 mL), and the resulting solution was dried over Na₂SO₄ and concentrated under reduced pressure. The residue was purified by flash chromatography on an ISCO 40 g gold column using CH₂Cl₂/CMA [CH₂Cl₂ (80%)/MeOH (18%)/NH₄OH (2%)](0-45% CMA) as the eluent to afford the free base of the title compound with reasonable purity. Further purification through column chromatography using CH₂Cl₂(A)/[CH₂Cl₂/MeOH(14% MeOH)](B) (0-65% of gradient solvent B) as the eluent afforded pure material which was converted to the hydrochloride using 2 N HCl in Et₂O (0.050 mL, 0.1 mmol). The resulting suspension was concentrated and lyophilized using water and CH₃CN to afford the title compound (29 mg, 6%) as an off-white solid: ¹H NMR (500 MHz, DMSO-d₆) δ 9.62 (br s, 1H), 9.42 (br s, 1H), 8.63 (m, 1H), 7.92 (t, J=7.7 Hz, 1H), 7.69 (m, 2H), 7.62 (m, 2H), 7.43 (t, J=6.7 Hz, 1H), 7.26 (m, 1H), 6.17 (m, 1H), 5.98 (s, 1H), 5.24 (s, 2H), 4.38 (m, 2H), 3.79 (m, 1H), 3.22 (dd, J=17.4 Hz, 12.7 Hz, 1H), 2.90 (dd, J=17.4 Hz, 7.9 Hz, 1H), 1.40 (d, J=6.56 Hz, 3H); ESI MS m/z 388 [M+H]⁺.

### Example 35

### Preparation of (S)-1-(1-Methyl-1,2,3,4-tetrahydrobenzofuro[3,2-c]pyridin-7-yl)-4-(pyridin-2-ylmethoxy)pyridin-2(1H)-one hydrochloride

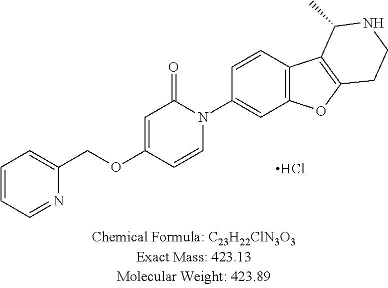 [SMILES: C[C@@H]1NCCc2oc3cc(-n4ccc(OCc5ccccn5)cc4=O)ccc3c21 ; Cl]

The free base of the title compound was obtained as a minor product in Example 34. The hydrochloride was prepared using 2 N HCl in Et₂O (0.025 mL, 0.05 mmol). The resulting suspension was concentrated and lyophilized using water and CH₃CN to afford the title compound (10.9 mg, 2.3%) as an off-white solid: ¹H NMR (500 MHz, DMSO-d₆) δ 9.67 (br s, 1H), 9.26 (br s, 1H), 8.63 (m, 1H), 7.91 (t, J=7.7 Hz, 1H), 7.69 (m, 2H), 7.62 (m, 2H), 7.42 (t, J=4.9 Hz, 1H), 7.26 (m, 1H), 6.17 (d, J=7.6 Hz, 1H), 5.98 (s, 1H), 5.23 (s, 2H), 4.79 (br s, 1H), 3.64 (m, 1H), 3.47 (m, 2H), 3.10 (m, 2H), 1.68 (d, J=6.81 Hz, 3H); ESI MS m/z 388 [M+H]⁺.

### Example 36

### Preparation of 1-(2,3,4,5-Tetrahydro-1H-benzofuro[3,2-c]azepin-8-yl)-4-(5-(trifluoromethyl)pyridin-2-yl)pyridin-2(1H)-one hydrochloride

### a) tert-Butyl 8-(2-oxo-4-(5-(trifluoromethyl)pyridin-2-yl)pyridin-1(2H)-yl)-4,5-dihydro-1H-benzofuro[3,2-c]azepine-2(3H)-carboxylate

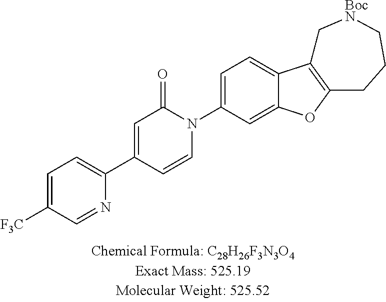 [SMILES: CC(C)(C)OC(=O)N1CCCc2oc3cc(-n4ccc(-c5ccc(C(F)(F)F)cn5)cc4=O)ccc3c2C1]

A mixture of 4-(5-(trifluoromethyl)pyridin-2-yl)pyridin-2(1H)-one (101 mg, 0.421 mmol), tert-butyl 8-bromo-4,5-dihydro-1H-benzofuro[3,2-c]azepine-2(3H)-carboxylate (128 mg, 0.349 mmol) and Cs₂CO₃ (148 mg, 0.454 mmol) in toluene (5 mL) in a sealed tube was degassed with a nitrogen stream for 10 min. Trans-N,N′-dimethylcyclohexane-1,2-diamine (75 mg, 0.53 mmol) and CuI (100 mg, 0.53 mmol) were added, and the mixture was degassed for another 2 min. The tube was sealed, and the mixture was heated at 110° C. for 12 h. The mixture was cooled, diluted with 90:9:1 CH₂Cl₂/MeOH/NH₄OH (15 mL) and stirred for 1 h. Then 2:1 brine/NH₄OH (100 mL) was added, and the aqueous phase was extracted with dichloromethane (4×75 mL). The combined organic extracts were washed with 2:1 brine/NH₄OH (3×75 mL), dried over Na₂SO₄ and concentrated under reduced pressure. Purification by flash chromatography (silica gel, CH₂Cl₂/(90:9:1 CH₂Cl₂/MeOH/NH₄OH), 100:0 to 25:75) afforded the title compound (62 mg, 34%) as an off-white solid: ESI MS m/z 526 [M+H]⁺.

### b) 1-(2,3,4,5-Tetrahydro-1H-benzofuro[3,2-c]azepin-8-yl)-4-(5-(trifluoromethyl)pyridin-2-yl)pyridin-2(1H)-one

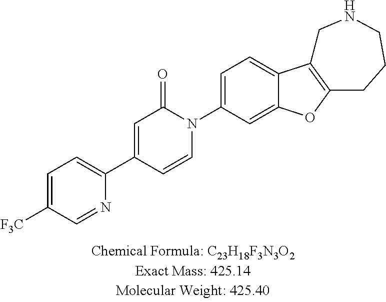 [SMILES: O=c1cc(-c2ccc(C(F)(F)F)cn2)ccn1-c1ccc2c3c(oc2c1)CCCNC3]

A solution of tert-butyl 8-(2-oxo-4-(5-(trifluoromethyl)pyridin-2-yl)pyridin-1(2H)-yl)-4,5-dihydro-1H-benzofuro[3,2-c]azepine-2(3H)-carboxylate (62 mg, 0.19 mmol) in MeOH (4 mL) was treated with 2 N HCl in Et₂O (1 mL), and the resulting solution was stirred at ambient temperature for 12 h. Additional 2 N HCl in Et₂O (0.2 mL) was added, and the resulting solution was stirred for another 4 h. Saturated aqueous NaHCO₃ (50 mL) was added, and the mixture was extracted with dichloromethane (8×50 mL). The combined organic extracts were dried over Na₂SO₄ and concentrated under reduced pressure. Purification by flash chromatography (silica gel, CH₂Cl₂/(90:9:1 CH₂Cl₂/MeOH/NH₄OH), 100:0 to 10:90) afforded the title compound (41 mg, 82%) as a light yellow solid: ESI MS m/z 426 [M+H]⁺.

### c) 1-(2,3,4,5-Tetrahydro-1H-benzofuro[3,2-c]azepin-8-yl)-4-(5-(trifluoromethyl)pyridin-2-yl)pyridin-2(1H)-one hydrochloride

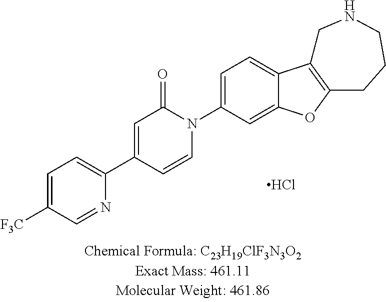 [SMILES: O=c1cc(-c2ccc(C(F)(F)F)cn2)ccn1-c1ccc2c3c(oc2c1)CCCNC3 ; Cl]

1-(2,3,4,5-Tetrahydro-1H-benzofuro[3,2-c]azepin-8-yl)-4-(5-(trifluoromethyl)pyridin-2-yl)pyridin-2(1H)-one (41 mg, 0.096 mmol) was suspended in MeOH (2 mL) and treated with 2 N HCl in Et₂O (49 μL, 0.098 mmol). The suspension was stirred at ambient temperature for 2 h, concentrated and lyophilized from acetonitrile-water to provide the title compound (48 mg, quant) as a white solid: ¹H NMR (500 MHz, DMSO-d₆) δ 9.28 (s, 2H), 9.15-9.14 (m, 1H), 8.41-8.34 (m, 2H), 7.86 (d, J=7.5 Hz, 1H), 7.82 (d, J=8.0 Hz, 1H), 7.76 (d, J=2.0 Hz, 1H), 7.39-7.37 (m, 1H), 7.29 (d, J=2.0 Hz, 1H), 7.10-7.08 (m, 1H), 4.43 (s, 2H), 3.50-3.48 (m, 2H), 3.13 (t, J=6.0 Hz, 2H), 2.12-2.08 (m, 2H); ESI MS m/z 426 [M+H]⁺; HPLC (Method B)>99% (AUC), t_{R}=13.8 min.

### Example 37

### Preparation of 4-((5-Fluoropyridin-2-yl)methoxy)-1-(2,3,4,5-tetrahydro-1H-benzofuro[3,2-c]azepin-8-yl)pyridin-2(1H)-one hydrochloride

### a) tert-Butyl 8-(4-((5-fluoropyridin-2-yl)methoxy)-2-oxopyridin-1(2H)-yl)-4,5-dihydro-1H-benzofuro[3,2-c]azepine-2(3H)-carboxylate

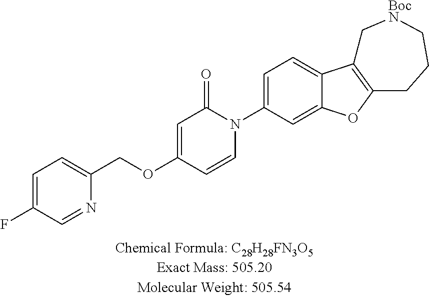 [SMILES: CC(C)(C)OC(=O)N1CCCc2oc3cc(-n4ccc(OCc5ccc(F)cn5)cc4=O)ccc3c2C1]

A mixture of 4-((5-fluoropyridin-2-yl)methoxy)pyridin-2(1H)-one (92 mg, 0.42 mmol), tert-butyl 8-bromo-4,5-dihydro-1H-benzofuro[3,2-c]azepine-2(3H)-carboxylate (128 mg, 0.349 mmol) and Cs₂CO₃ (148 mg, 0.454 mmol) in toluene (5 mL) in a sealed tube was degassed with a nitrogen stream for 10 min. Trans-N,N′-dimethylcyclohexane-1,2-diamine (75 mg, 0.53 mmol) and CuI (100 mg, 0.53 mmol) were added, and the mixture was degassed for another 2 min. The tube was sealed, and the mixture was heated at 110° C. for 12 h. The mixture was cooled, diluted with 90:9:1 CH₂Cl₂/MeOH/NH₄OH (15 mL) and stirred for 1 h. Then 2:1 brine/NH₄OH (100 mL) was added, and the aqueous phase was extracted with dichloromethane (4×75 mL). The combined organic extracts were washed with 2:1 brine/NH₄OH (3×75 mL), dried over Na₂SO₄ and concentrated under reduced pressure. Purification by flash chromatography (silica gel, CH₂Cl₂/(90:9:1 CH₂Cl₂/MeOH/NH₄OH), 100:0 to 25:75) afforded the title compound (77 mg, 44%) as a white solid: ESI MS m/z 506 [M+H]⁺.

### b) 4-((5-Fluoropyridin-2-yl)methoxy)-1-(2,3,4,5-tetrahydro-1H-benzofuro[3,2-c]azepin-8-yl)pyridin-2(1H)-one

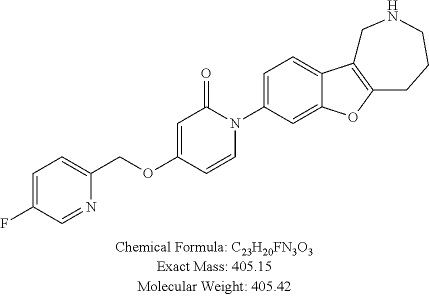 [SMILES: O=c1cc(OCc2ccc(F)cn2)ccn1-c1ccc2c3c(oc2c1)CCCNC3]

A solution of tert-butyl 8-(4-((5-fluoropyridin-2-yl)methoxy)-2-oxopyridin-1(2H)-yl)-4,5-dihydro-1H-benzofuro[3,2-c]azepine-2(3H)-carboxylate (77 mg, 0.15 mmol) in MeOH (4 mL) was treated with 2 N HCl in Et₂O (1 mL), and the resulting solution was stirred at ambient temperature for 12 h. Additional 2 N HCl in Et₂O (0.2 mL) was added, and the resulting solution was stirred for another 4 h. Saturated aqueous NaHCO₃ (50 mL) was added, and the mixture was extracted with dichloromethane (8×50 mL). The combined organic extracts were dried over Na₂SO₄ and concentrated under reduced pressure. Purification by flash chromatography (silica gel, CH₂Cl₂/(90:9:1 CH₂Cl₂/MeOH/NH₄OH), 100:0 to 10:90) afforded the title compound (59 mg, 96%) as a white solid: ESI MS m/z 406 [M+H]⁺.

### c) 4-((5-Fluoropyridin-2-yl)methoxy)-1-(2,3,4,5-tetrahydro-1H-benzofuro[3,2-c]azepin-8-yl)pyridin-2(1H)-one hydrochloride

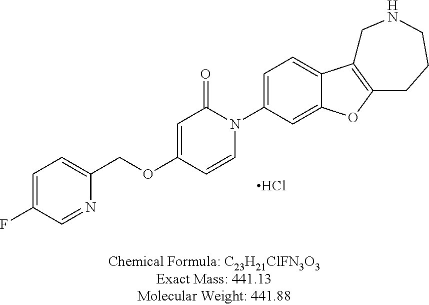 [SMILES: O=c1cc(OCc2ccc(F)cn2)ccn1-c1ccc2c3c(oc2c1)CCCNC3 ; Cl]

4-((5-Fluoropyridin-2-yl)methoxy)-1-(2,3,4,5-tetrahydro-1H-benzofuro[3,2-c]azepin-8-yl)pyridin-2(1H)-one (59 mg, 0.15 mmol) was suspended in MeOH (2 mL) and treated with 2 N HCl in Et₂O (73 μL, 0.15 mmol). The suspension was stirred at ambient temperature for 2 h, concentrated and lyophilized from acetonitrile-water to provide the title compound (62 mg, 96%) as a white solid: ¹H NMR (500 MHz, DMSO-d₆) δ 9.34 (s, 2H), 8.62 (d, J=2.5 Hz, 1H), 7.85-7.81 (m, 1H), 7.76 (d, J=8.0 Hz, 1H), 7.67-7.64 (m, 1H), 7.62-7.59 (m, 2H), 7.25 (d, J=8.0 Hz, 1H), 6.16-6.14 (m, 1H), 5.98 (d, J=8.0 Hz, 1H), 5.22 (s, 2H), 4.41 (s, 2H), 3.47 (s, 2H), 3.11 (t, J=6.0 Hz, 2H), 2.10-2.07 (m, 2H); ESI MS m/z 406 [M+H]⁺; HPLC (Method B)>99% (AUC), t_{R}=12.4 min.

### Example 38

### Preparation of 4-((5-Chloropyridin-2-yl)methoxy)-1-(2,3,4,5-tetrahydro-1H-benzofuro[3,2-c]azepin-8-yl)pyridin-2(1H)-one hydrochloride

### a) tert-Butyl 8-(4-((5-chloropyridin-2-yl)methoxy)-2-oxopyridin-1(2H)-yl)-4,5-dihydro-1H-benzofuro[3,2-c]azepine-2(3H)-carboxylate

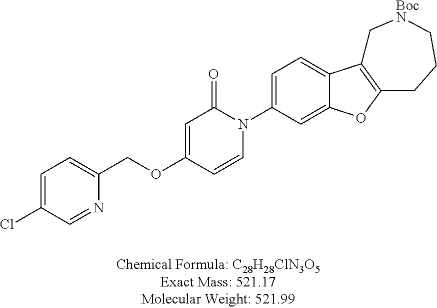 [SMILES: CC(C)(C)OC(=O)N1CCCc2oc3cc(-n4ccc(OCc5ccc(Cl)cn5)cc4=O)ccc3c2C1]

A mixture of 4-((5-Chloropyridin-2-yl)methoxy)pyridin-2(1H)-one (99 mg, 0.42 mmol), tert-butyl 8-bromo-4,5-dihydro-1H-benzofuro[3,2-c]azepine-2(3H)-carboxylate (128 mg, 0.349 mmol) and Cs₂CO₃ (148 mg, 0.454 mmol) in toluene (5 mL) in a sealed tube was degassed with a nitrogen stream for 10 min. Trans-N,N′-dimethylcyclohexane-1,2-diamine (75 mg, 0.53 mmol) and CuI (100 mg, 0.53 mmol) were added, and the mixture was degassed for another 2 min. The tube was sealed, and the mixture was heated at 110° C. for 12 h. The mixture was cooled, diluted with 90:9:1 CH₂Cl₂/MeOH/NH₄OH (15 mL) and stirred for 1 h. Then 2:1 brine/NH₄OH (100 mL) was added, and the aqueous phase was extracted with dichloromethane (4×75 mL). The combined organic extracts were washed with 2:1 brine/NH₄OH (3×75 mL), dried over Na₂SO₄ and concentrated under reduced pressure. Purification by flash chromatography (silica gel, CH₂Cl₂/(90:9:1 CH₂Cl₂/MeOH/NH₄OH), 100:0 to 25:75) afforded the title compound (58 mg, 32%) as an off-white solid: ESI MS m/z 522 [M+H]⁺.

### b) 4-((5-Chloropyridin-2-yl)methoxy)-1-(2,3,4,5-tetrahydro-1H-benzofuro[3,2-c]azepin-8-yl)pyridin-2(1H)-one

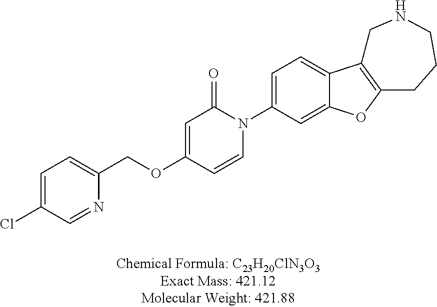 [SMILES: O=c1cc(OCc2ccc(Cl)cn2)ccn1-c1ccc2c3c(oc2c1)CCCNC3]

A solution of tert-butyl 8-(4-((5-chloropyridin-2-yl)methoxy)-2-oxopyridin-1(2H)-yl)-4,5-dihydro-1H-benzofuro[3,2-c]azepine-2(3H)-carboxylate (58 mg, 0.11 mmol) in MeOH (4 mL) was treated with 2 N HCl in Et₂O (1 mL), and the resulting solution was stirred at ambient temperature for 12 h. Additional 2 N HCl in Et₂O (0.2 mL) was added, and the resulting solution was stirred for another 4 h. Saturated aqueous NaHCO₃ (50 mL) was added, and the mixture was extracted with dichloromethane (8×50 mL). The combined organic extracts were dried over Na₂SO₄ and concentrated under reduced pressure. Purification by flash chromatography (silica gel, CH₂Cl₂/(90:9:1 CH₂Cl₂/MeOH/NH₄OH), 100:0 to 10:90) afforded the title compound (45 mg, 95%) as a white solid: ESI MS m/z 422 [M+H]⁺.

### c) 4-((5-Chloropyridin-2-yl)methoxy)-1-(2,3,4,5-tetrahydro-1H-benzofuro[3,2-c]azepin-8-yl)pyridin-2(1H)-one hydrochloride

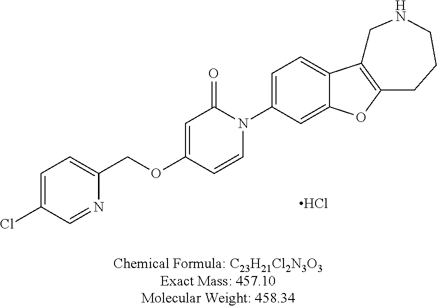 [SMILES: O=c1cc(OCc2ccc(Cl)cn2)ccn1-c1ccc2c3c(oc2c1)CCCNC3 ; Cl]

4-((5-Chloropyridin-2-yl)methoxy)-1-(2,3,4,5-tetrahydro-1H-benzofuro[3,2-c]azepin-8-yl)pyridin-2(1H)-one (45 mg, 0.11 mmol) was suspended in MeOH (2 mL) and treated with 2 N HCl in Et₂O (53 μL, 0.11 mmol). The suspension was stirred at ambient temperature for 2 h, concentrated and lyophilized from acetonitrile-water to provide the title compound (47 mg, 97%) as a white solid: ¹H NMR (500 MHz, DMSO-d₆) δ 9.23 (s, 2H), 8.68-8.67 (m, 1H), 8.04-8.02 (m, 1H), 7.75 (d, J=8.5 Hz, 1H), 7.63-7.60 (m, 3H), 7.26-7.24 (m, 1H), 6.18-6.16 (m, 1H), 5.97 (d, J=3.0 Hz, 1H), 5.24 (s, 2H), 4.41 (s, 2H), 3.49-3.47 (m, 2H), 3.11 (t, J=6.0 Hz, 2H). 2.11-2.07 (m, 2H); ESI MS m/z 422 [M+H]⁺; HPLC (Method B)>99% (AUC), t_{R}=13.0 min.

### Example 39

### Preparation of 4-((5-Fluoropyridin-2-yl)methoxy)-1-(2,3,4,5-tetrahydro-1H-benzofuro[2,3-d]azepin-8-yl)pyridin-2(1H)-one hydrochloride

### a) tert-Butyl 8-(4-((5-fluoropyridin-2-yl)methoxy)-2-oxopyridin-1(2H)-yl)-4,5-dihydro-1H-benzofuro[2,3-d]azepine-3(2H)-carboxylate

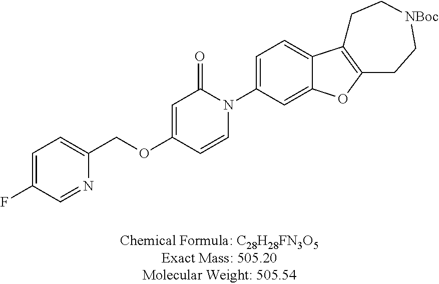 [SMILES: CC(C)(C)OC(=O)N1CCc2oc3cc(-n4ccc(OCc5ccc(F)cn5)cc4=O)ccc3c2CC1]

A mixture of 4-((5-fluoropyridin-2-yl)methoxy)pyridin-2(1H)-one (139 mg, 0.631 mmol), tert-butyl 8-bromo-4,5-dihydro-1H-benzofuro[2,3-d]azepine-3(2H)-carboxylate (192 mg, 0.524 mmol) and Cs₂CO₃ (222 mg, 0.681 mmol) in toluene (6 mL) in a sealed tube was degassed with a nitrogen stream for 10 min. Trans-N,N′-dimethylcyclohexane-1,2-diamine (149 mg, 1.05 mmol) and CuI (200 mg, 1.05 mmol) were added, and the mixture was degassed for another 2 min. The tube was sealed, and the mixture was heated at 110° C. for 12 h. The mixture was cooled, diluted with 90:9:1 CH₂Cl₂/MeOH/NH₄OH (15 mL) and stirred for 1 h. Then 2:1 brine/NH₄OH (100 mL) was added and the aqueous phase was extracted with dichloromethane (4×75 mL). The combined organic extracts were washed with 2:1 brine/NH₄OH (3×75 mL), dried over Na₂SO₄ and concentrated under reduced pressure. Purification by flash chromatography (silica gel, CH₂Cl₂/(90:9:1 CH₂Cl₂/MeOH/NH₄OH), 100:0 to 25:75) afforded the title compound (60 mg, 23%) as a white solid: ESI MS m/z 506 [M+H]⁺.

### b) 4-((5-Fluoropyridin-2-yl)methoxy)-1-(2,3,4,5-tetrahydro-1H-benzofuro[2,3-d]azepin-8-yl)pyridin-2(1H)-one

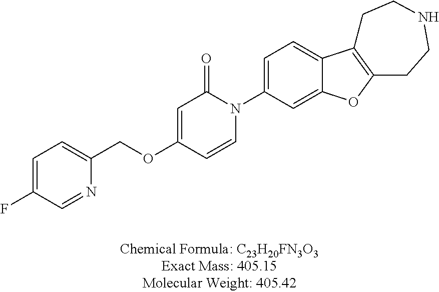 [SMILES: O=c1cc(OCc2ccc(F)cn2)ccn1-c1ccc2c3c(oc2c1)CCNCC3]

A solution of tert-butyl 8-(4-((5-fluoropyridin-2-yl)methoxy)-2-oxopyridin-1(2H)-yl)-4,5-dihydro-1H-benzofuro[2,3-d]azepine-3(2H)-carboxylate (60 mg, 0.12 mmol) in MeOH (3 mL) was treated with 2 N HCl in Et₂O (1.5 mL), and the resulting solution was stirred at ambient temperature for 12 h. Additional 2 N HCl in Et₂O (0.2 mL) was added, and the resulting solution was stirred for another 4 h. Saturated aqueous NaHCO₃ (50 mL) was added, and the mixture was extracted with dichloromethane (8×50 mL). The combined organic extracts were dried over Na₂SO₄ and concentrated under reduced pressure. Purification by flash chromatography (silica gel, CH₂Cl₂/(90:9:1 CH₂Cl₂/MeOH/NH₄OH), 100:0 to 10:90) afforded the title compound (29 mg, 60%) as a white solid: ESI MS m/z 406 [M+H]⁺.

### c) 4-((5-Fluoropyridin-2-yl)methoxy)-1-(2,3,4,5-tetrahydro-1H-benzofuro[2,3-d]azepin-8-yl)pyridin-2(1H)-one hydrochloride

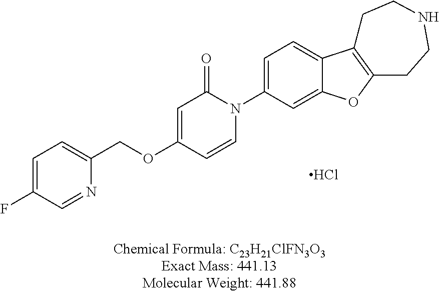 [SMILES: O=c1cc(OCc2ccc(F)cn2)ccn1-c1ccc2c3c(oc2c1)CCNCC3 ; Cl]

4-((5-Fluoropyridin-2-yl)methoxy)-1-(2,3,4,5-tetrahydro-1H-benzofuro[2,3-d]azepin-8-yl)pyridin-2(1H)-one (29 mg, 0.072 mmol) was suspended in MeOH (1.5 mL) and treated with 2 N HCl in Et₂O (33 μL, 0.066 mmol). The suspension was stirred at ambient temperature for 2 h, concentrated and lyophilized from acetonitrile-water to provide the title compound (31 mg, 99%) as a white solid: ¹H NMR (500 MHz, DMSO-d₆) δ 9.43 (s, 2H), 8.62 (d, J=3.0 Hz, 1H), 7.85-7.81 (m, 1H), 7.67-7.63 (m, 2H), 7.60-7.58 (m, 2H), 7.23-7.21 (m, 1H), 6.16-6.14 (m, 1H), 5.98 (d, J=2.5 Hz, 1H), 5.22 (s, 2H), 3.43-3.37 (m, 4H), 3.30-3.28 (m, 2H), 3.08 (t, J=5.5 Hz, 2H); ESI MS m/z 406 [M+H]⁺; HPLC (Method B)>99% (AUC), t_{R}=12.4 min.

### Example 40

### Preparation of 4-(Benzyloxy)-1-(2,3,4,5-tetrahydro-1H-benzofuro[3,2-c]azepin-8-yl)pyridin-2(1H)-one hydrochloride

### a) tert-Butyl 8-(4-(benzyloxy)-2-oxopyridin-1(2H)-yl)-4,5-dihydro-1H-benzofuro[3,2-c]azepine-2(3H)-carboxylate

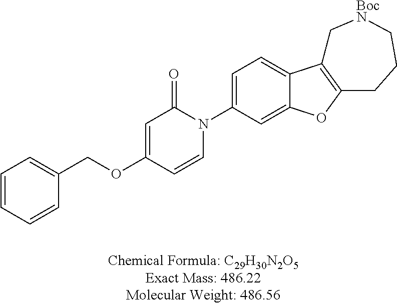 [SMILES: CC(C)(C)OC(=O)N1CCCc2oc3cc(-n4ccc(OCc5ccccc5)cc4=O)ccc3c2C1]

A mixture of 4-(benzyloxy)pyridin-2(1H)-one (85 mg, 0.42 mmol), tert-butyl 8-bromo-4,5-dihydro-1H-benzofuro[3,2-c]azepine-2(3H)-carboxylate (128 mg, 0.349 mmol) and Cs₂CO₃ (148 mg, 0.454 mmol) in toluene (6 mL) in a sealed tube was degassed with a nitrogen stream for 10 min. Trans-N,N′-dimethylcyclohexane-1,2-diamine (75 mg, 0.53 mmol) and CuI (100 mg, 0.53 mmol) were added, and the mixture was degassed for another 2 min. The tube was sealed, and the mixture was heated at 110° C. for 12 h. The mixture was cooled, diluted with 90:9:1 CH₂Cl₂/MeOH/NH₄OH (15 mL) and stirred for 1 h. Then 2:1 brine/NH₄OH (100 mL) was added, and the aqueous phase was extracted with dichloromethane (4×75 mL). The combined organic extracts were washed with 2:1 brine/NH₄OH (3×75 mL), dried over Na₂SO₄ and concentrated under reduced pressure. Purification by flash chromatography (silica gel, CH₂Cl₂/(90:9:1 CH₂Cl₂/MeOH/NH₄OH), 100:0 to 25:75) afforded the title compound (35 mg, 21%) as a white solid: ESI MS m/z 487 [M+H]⁺.

### b) 4-(Benzyloxy)-1-(2,3,4,5-tetrahydro-1H-benzofuro[3,2-c]azepin-8-yl)pyridin-2(1H)-one

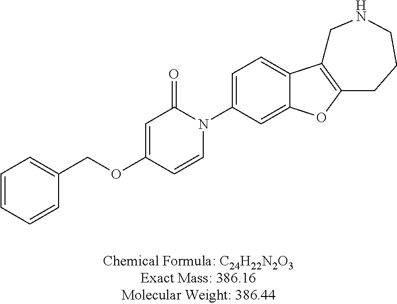 [SMILES: O=c1cc(OCc2ccccc2)ccn1-c1ccc2c3c(oc2c1)CCCNC3]

A solution of tert-butyl 8-(4-(benzyloxy)-2-oxopyridin-1(2H)-yl)-4,5-dihydro-1H-benzofuro[3,2-c]azepine-2(3H)-carboxylate (35 mg, 0.072 mmol) in MeOH (2 mL) was treated with 2 N HCl in Et₂O (1 mL), and the resulting solution was stirred at ambient temperature for 12 h. Additional 2 N HCl in Et₂O (0.2 mL) was added, and the resulting solution was stirred for another 4 h. Saturated aqueous NaHCO₃ (50 mL) was added, and the mixture was extracted with dichloromethane (8×50 mL). The combined organic extracts were dried over Na₂SO₄ and concentrated under reduced pressure. Purification by flash chromatography (silica gel, CH₂Cl₂/(90:9:1 CH₂Cl₂/MeOH/NH₄OH), 100:0 to 10:90) afforded the title compound (23 mg, 83%) as a white solid: ESI MS m/z 387 [M+H]⁺.

### c) 4-(Benzyloxy)-1-(2,3,4,5-tetrahydro-1H-benzofuro[3,2-c]azepin-8-yl)pyridin-2(1H)-one hydrochloride

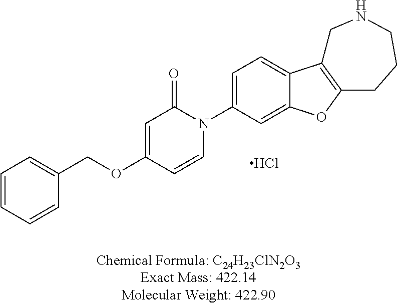 [SMILES: O=c1cc(OCc2ccccc2)ccn1-c1ccc2c3c(oc2c1)CCCNC3 ; Cl]

4-(Benzyloxy)-1-(2,3,4,5-tetrahydro-1H-benzofuro[3,2-c]azepin-8-yl)pyridin-2(1H)-one (23 mg, 0.060 mmol) was suspended in MeOH (1 mL) and treated with 2 N HCl in Et₂O (30 μL, 0.060 mmol). The suspension was stirred at ambient temperature for 2 h, concentrated and lyophilized from acetonitrile-water to provide the title compound (25 mg, quant) as a white solid: ¹H NMR (500 MHz, DMSO-d₆) δ 9.35 (s, 2H), 7.75 (d, J=8.5 Hz, 1H), 7.61 (d, J=2.0 Hz, 1H), 7.59 (d, J=7.5 Hz, 1H), 7.48-7.41 (m, 4H), 7.39-7.36 (m, 1H), 7.26 (dd, J=8.5, 2.0 Hz, 1H), 6.12 (dd. J=7.5, 2.5 Hz, 1H), 5.98 (d, J=2.5 Hz, 1H), 5.15 (s, 2H), 4.40 (s, 2H), 3.48-3.46 (m, 2H), 3.11 (t, J=5.5 Hz, 2H), 2.11-2.07 (m, 2H); ESI MS m/z 387 [M+H]⁺; HPLC (Method B)>99% (AUC), t_{R}=13.8 min.

### Example 41

### Preparation of 4-(Benzyloxy)-1-(2,3,4,5-tetrahydro-1H-benzofuro[2,3-d]azepin-8-yl)pyridin-2(1H)-one hydrochloride

### a) tert-Butyl 8-(4-(benzyloxy)-2-oxopyridin-1(2H)-yl)-4,5-dihydro-1H-benzofuro[2,3-d]azepine-3(2H)-carboxylate

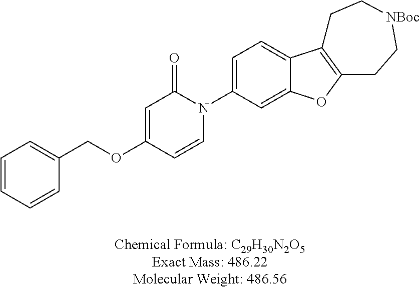 [SMILES: CC(C)(C)OC(=O)N1CCc2oc3cc(-n4ccc(OCc5ccccc5)cc4=O)ccc3c2CC1]

A mixture of 4-(benzyloxy)pyridin-2(1H)-one (204 mg, 1.02 mmol), tert-butyl 8-bromo-4,5-dihydro-1H-benzofuro[2,3-d]azepine-3(2H)-carboxylate (310 mg, 0.85 mmol) and Cs₂CO₃ (358 mg, 1.10 mmol) in toluene (7 mL) in a sealed tube was degassed with a nitrogen stream for 10 min. Trans-N,N′-dimethylcyclohexane-1,2-diamine (241 mg, 1.69 mmol) and CuI (322 mg, 1.69 mmol) were added, and the mixture was degassed for another 2 min. The tube was sealed, and the mixture was heated at 110° C. for 12 h. The mixture was cooled, diluted with 90:9:1 CH₂Cl₂/MeOH/NH₄OH (15 mL) and stirred for 1 h. Then 2:1 brine/NH₄OH (100 mL) was added, and the aqueous phase was extracted with dichloromethane (4×75 mL). The combined organic extracts were washed with 2:1 brine/NH₄OH (3×75 mL), dried over Na₂SO₄ and concentrated under reduced pressure. Purification by flash chromatography (silica gel, CH₂Cl₂/(90:9:1 CH₂Cl₂/MeOH/NH₄OH), 100:0 to 25:75) afforded the title compound (139 mg, 34%) as a light yellow solid: ESI MS m/z 487 [M+H]⁺.

### b) 4-(Benzyloxy)-1-(2,3,4,5-tetrahydro-1H-benzofuro[2,3-d]azepin-8-yl)pyridin-2(1H)-one

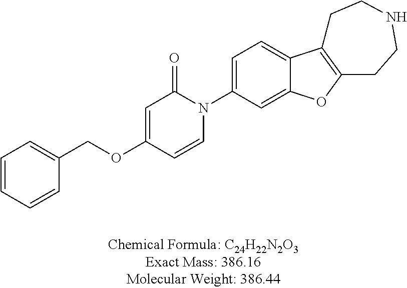 [SMILES: O=c1cc(OCc2ccccc2)ccn1-c1ccc2c3c(oc2c1)CCNCC3]

A solution of tert-butyl 8-(4-(benzyloxy)-2-oxopyridin-1(2H)-yl)-4,5-dihydro-1H-benzofuro[2,3-d]azepine-3(2H)-carboxylate (135 mg, 0.277 mmol) in MeOH (5 mL) was treated with 2 N HCl in Et₂O (2.5 mL), and the resulting solution was stirred at ambient temperature for 12 h. Additional 2 N HCl in Et₂O (0.2 mL) was added, and the resulting solution was stirred for another 4 h. Saturated aqueous NaHCO₃ (50 mL) was added, and the mixture was extracted with dichloromethane (8×50 mL). The combined organic extracts were dried over Na₂SO₄ and concentrated under reduced pressure. Purification by flash chromatography (silica gel, CH₂Cl₂/(90:9:1 CH₂Cl₂/MeOH/NH₄OH), 100:0 to 10:90) afforded the title compound (79 mg, 74%) as a white solid: ESI MS m/z 387 [M+H]⁺.

### c) 4-(Benzyloxy)-1-(2,3,4,5-tetrahydro-1H-benzofuro[2,3-d]azepin-8-yl)pyridin-2(1H)-one hydrochloride

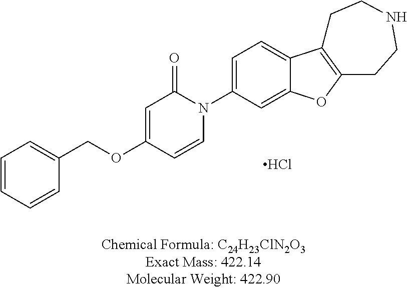 [SMILES: O=c1cc(OCc2ccccc2)ccn1-c1ccc2c3c(oc2c1)CCNCC3 ; Cl]

4-(Benzyloxy)-1-(2,3,4,5-tetrahydro-1H-benzofuro[2,3-d]azepin-8-yl)pyridin-2(1H)-one (76 mg, 0.20 mmol) was suspended in MeOH (3 mL) and treated with 2 N HCl in Et₂O (98 μL, 0.20 mmol). The suspension was stirred at ambient temperature for 2 h, concentrated and lyophilized from acetonitrile-water to provide the title compound (83 mg, quant) as a white solid: ¹H NMR (500 MHz, DMSO-d₆) δ 9.42 (s, 2H), 7.63 (d, J=8.0 Hz, 1H), 7.58-7.57 (m, 2H), 7.48-7.37 (m, 4H), 7.39-7.37 (m, 1H), 7.22 (dd, J=8.5, 2.0 Hz, 1H), 6.11 (dd, J=7.5, 2.5 Hz, 1H), 5.98 (d, J=2.5 Hz, 1H), 5.15 (s, 2H), 3.43-3.37 (m, 4H), 3.30-3.37 (m, 2H), 3.07 (t, J=5.5 Hz, 2H); ESI MS m/z 387 [M+H]⁺; HPLC (Method B)>99% (AUC), t_{R}=13.9 min.

### Example 42

### Preparation of 4-((5-Chloropyridin-2-yl)methoxy)-1-(2,3,4,5-tetrahydro-1H-benzofuro[2,3-d]azepin-8-yl)pyridin-2(1H)-one hydrochloride

### a) tert-Butyl 8-(4-((5-chloropyridin-2-yl)methoxy)-2-oxopyridin-1(2H)-yl)-4,5-dihydro-1H-benzofuro[2,3-d]azepine-3(2H)-carboxylate

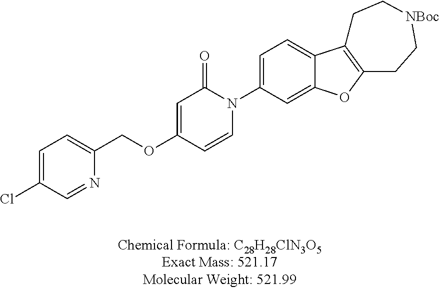 [SMILES: CC(C)(C)OC(=O)N1CCc2oc3cc(-n4ccc(OCc5ccc(Cl)cn5)cc4=O)ccc3c2CC1]

A mixture of 4-((5-chloropyridin-2-yl)methoxy)pyridin-2(1H)-one (240 mg, 1.02 mmol), tert-butyl 8-bromo-4,5-dihydro-1H-benzofuro[2,3-d]azepine-3(2H)-carboxylate (310 mg, 0.85 mmol) and Cs₂CO₃ (358 mg, 1.10 mmol) in toluene (7 mL) in a sealed tube was degassed with a nitrogen stream for 10 min. Trans-N,N′-dimethylcyclohexane-1,2-diamine (241 mg, 1.69 mmol), CuI (322 mg, 1.69 mmol) were added, and the mixture was degassed for another 2 min. The tube was sealed, and the mixture was heated at 110° C. for 12 h. The mixture was cooled, diluted with 90:9:1 CH₂Cl₂/MeOH/NH₄OH (15 mL) and stirred for 1 h. Then 2:1 brine/NH₄OH (100 mL) was added, and the aqueous phase was extracted with dichloromethane (4×75 mL). The combined organic extracts were washed with 2:1 brine/NH₄OH (3×75 mL), dried over Na₂SO₄ and concentrated under reduced pressure. Purification by flash chromatography (silica gel, CH₂Cl₂/(90:9:1 CH₂Cl₂/MeOH/NH₄OH), 100:0 to 25:75) afforded the title compound (123 mg, 28%) as a light yellow oil: ESI MS m/z 522 [M+H]⁺.

### b) 4-((5-Chloropyridin-2-yl)methoxy)-1-(2,3,4,5-tetrahydro-1H-benzofuro[2,3-d]azepin-8-yl)pyridin-2(1H)-one

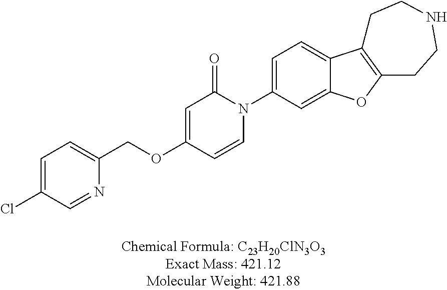 [SMILES: O=c1cc(OCc2ccc(Cl)cn2)ccn1-c1ccc2c3c(oc2c1)CCNCC3]

A solution of tert-butyl 8-(4-((5-chloropyridin-2-yl)methoxy)-2-oxopyridin-1(2H)-yl)-4,5-dihydro-1H-benzofuro[2,3-d]azepine-3(2H)-carboxylate (120 mg, 0.23 mmol) in MeOH (4 mL) was treated with 2 N HCl in Et₂O (2 mL), and the resulting solution was stirred at ambient temperature for 12 h. Additional 2 N HCl in Et₂O (0.2 mL) was added, and the resulting solution was stirred for another 4 h. Saturated aqueous NaHCO₃ (50 mL) was added, and the mixture was extracted with dichloromethane (8×50 mL). The combined organic extracts were dried over Na₂SO₄ and concentrated under reduced pressure. Purification by flash chromatography (silica gel, CH₂Cl₂/(90:9:1 CH₂Cl₂/MeOH/NH₄OH), 100:0 to 10:90) afforded the title compound (64 mg, 66%) as a white solid: ESI MS m/z 422 [M+H]⁺.

### c) 4-((5-Chloropyridin-2-yl)methoxy)-1-(2,3,4,5-tetrahydro-1H-benzofuro[2,3-d]azepin-8-yl)pyridin-2(1H)-one hydrochloride

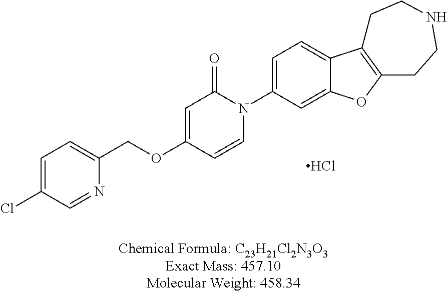 [SMILES: O=c1cc(OCc2ccc(Cl)cn2)ccn1-c1ccc2c3c(oc2c1)CCNCC3 ; Cl]

4-((5-Chloropyridin-2-yl)methoxy)-1-(2,3,4,5-tetrahydro-1H-benzofuro[2,3-d]azepin-8-yl)pyridin-2(1H)-one (61 mg, 0.14 mmol) was suspended in MeOH (2 mL) and treated with 2 N HCl in Et₂O (72 μL, 0.14 mmol). The suspension was stirred at ambient temperature for 2 h, concentrated and lyophilized from acetonitrile-water to provide the title compound (66 mg, quant) as a white solid: ¹H NMR (500 MHz, DMSO-d₆) δ 9.43 (s, 2H), 8.67 (d, J=2.5 Hz, 1H), 8.03 (dd, J=8.5, 2.5 Hz, 1H), 7.64-7.58 (m, 4H), 7.22 (dd, J=8.0, 2.0 Hz, 1H), 6.16 (dd, J=8.0, 2.0 Hz, 1H), 5.96 (d, J=3.0 Hz, 1H), 5.23 (s, 2H), 3.43-3.37 (m, 4H), 3.30-3.28 (m, 2H), 3.09-3.06 (m, 2H); ESI MS m/z 422 [M+H]⁺; HPLC (Method B)>99% (AUC), t_{R}=13.2 min.

### Example 43

### Preparation of 1-(2,3,4,5-Tetrahydro-1H-benzofuro[3,2-c]azepin-8-yl)-4-((6-(trifluoromethyl)pyridin-2-yl)methoxy)pyridin-2(1H)-one hydrochloride

### a) tert-Butyl 8-(2-oxo-4-((6-(trifluoromethyl)pyridin-2-yl)methoxy)pyridin-1(2H)-yl)-4,5-dihydro-1H-benzofuro[3,2-c]azepine-2(3H)-carboxylate

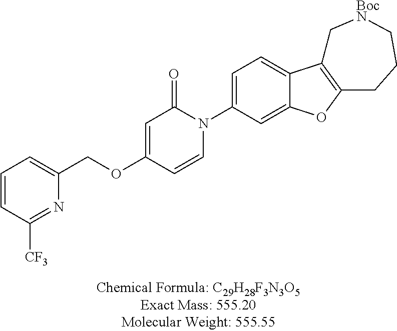 [SMILES: CC(C)(C)OC(=O)N1CCCc2oc3cc(-n4ccc(OCc5cccc(C(F)(F)F)n5)cc4=O)ccc3c2C1]

A mixture of 4-((6-(trifluoromethyl)pyridin-2-yl)methoxy)pyridin-2(1H)-one (170 mg, 0.63 mmol), tert-butyl 8-bromo-4,5-dihydro-1H-benzofuro[3,2-c]azepine-2(3H)-carboxylate (192 mg, 0.524 mmol) and Cs₂CO₃ (222 mg, 0.681 mmol) in toluene (6 mL) in a sealed tube was degassed with a nitrogen stream for 10 min. Trans-N,N′-dimethylcyclohexane-1,2-diamine (149 mg, 1.05 mmol) and CuI (200 mg, 1.05 mmol) were added, and the mixture was degassed for another 2 min. The tube was sealed, and the mixture was heated at 110° C. for 12 h. The mixture was cooled, diluted with 90:9:1 CH₂Cl₂/MeOH/NH₄OH (15 mL) and stirred for 1 h. Then 2:1 brine/NH₄OH (100 mL) was added, and the aqueous phase was extracted with dichloromethane (4×75 mL). The combined organic extracts were washed with 2:1 brine/NH₄OH (3×75 mL), dried over Na₂SO₄ and concentrated under reduced pressure. Purification by flash chromatography (silica gel, CH₂Cl₂/(90:9:1 CH₂Cl₂/MeOH/NH₄OH), 100:0 to 25:75) afforded the title compound (229 mg, 79%) as a white solid: ESI MS m/z 556 [M+H]⁺.

### b) 1-(2,3,4,5-Tetrahydro-1H-benzofuro[3,2-c]azepin-8-yl)-4-((6-(trifluoromethyl)pyridin-2-yl)methoxy)pyridin-2(1H)-one

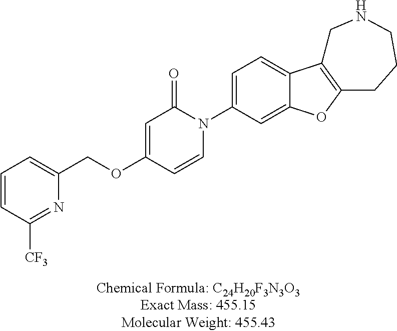 [SMILES: O=c1cc(OCc2cccc(C(F)(F)F)n2)ccn1-c1ccc2c3c(oc2c1)CCCNC3]

A solution of tert-butyl 8-(2-oxo-4-((6-(trifluoromethyl)pyridin-2-yl)methoxy)pyridin-1(2H)-yl)-4,5-dihydro-1H-benzofuro[3,2-c]azepine-2(3H)-carboxylate (225 mg, 0.405 mmol) in MeOH (6 mL) was treated with 2 N HCl in Et₂O (2.5 mL), and the resulting solution was stirred at ambient temperature for 12 h. Additional 2 N HCl in Et₂O (0.2 mL) was added, and the resulting solution was stirred for another 4 h. Saturated aqueous NaHCO₃ (50 mL) was added, and the mixture was extracted with dichloromethane (8×50 mL). The combined organic extracts were dried over Na₂SO₄ and concentrated under reduced pressure. Purification by flash chromatography (silica gel, CH₂Cl₂/(90:9:1 CH₂Cl₂/MeOH/NH₄OH), 100:0 to 10:90) afforded the title compound (161 mg, 87%) as a white solid: ESI MS m/z 456 [M+H]⁺.

### c) 1-(2,3,4,5-Tetrahydro-1H-benzofuro[3,2-c]azepin-8-yl)-4-((6-(trifluoromethyl)pyridin-2-yl)methoxy)pyridin-2(1H)-one hydrochloride

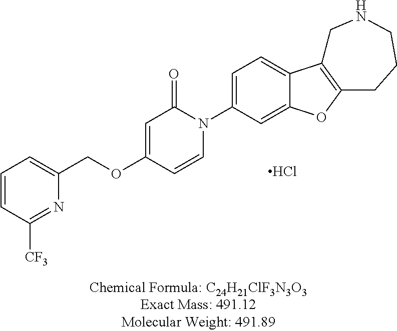 [SMILES: O=c1cc(OCc2cccc(C(F)(F)F)n2)ccn1-c1ccc2c3c(oc2c1)CCCNC3 ; Cl]

1-(2,3,4,5-Tetrahydro-1H-benzofuro[3,2-c]azepin-8-yl)-4-((6-(trifluoromethyl)pyridin-2-yl)methoxy)pyridin-2(1H)-one (158 mg, 0.347 mmol) was suspended in MeOH (4 mL) and treated with 2 N HCl in Et₂O (17 μL, 0.34 mmol). The suspension was stirred at ambient temperature for 2 h, concentrated and lyophilized from acetonitrile-water to provide the title compound (166 mg, 98%) as a white solid: ¹H NMR (500 MHz, DMSO-d₆) δ 9.37 (s, 2H), 8.21 (t, J=8.0 Hz, 1H), 7.94-7.88 (m, 2H), 7.76 (d, J=8.0 Hz, 1H), 7.64-7.62 (m, 2H), 7.25 (dd, J=8.0, 2.0 Hz, 1H), 6.20 (dd, J=7.5, 2.5 Hz, 1H), 6.00 (d, J=2.5 Hz, 1H), 5.33 (s, 2H), 4.41 (s, 2H), 3.49-3.45 (m, 2H), 3.11 (t, J=5.5 Hz, 2H), 2.12-1.96 (m, 2H); ESI MS m/z 456 [M+H]⁺; HPLC (Method B) 95.8% (AUC), t_{R}=13.9 min.

### Example 44

### Preparation of 5-((5-Fluoropyridin-2-yl)methoxy)-2-(2,3,4,5-tetrahydro-1H-benzofuro[3,2-c]azepin-8-yl)pyridazin-3(2H)-one hydrochloride

### a) tert-Butyl 8-(4-((5-fluoropyridin-2-yl)methoxy)-6-oxopyridazin-1(6H)-yl)-4,5-dihydro-1H-benzofuro[3,2-c]azepine-2(3H)-carboxylate

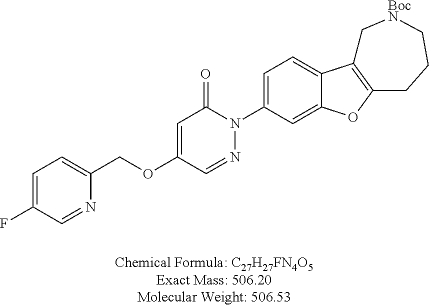 [SMILES: CC(C)(C)OC(=O)N1CCCc2oc3cc(-n4ncc(OCc5ccc(F)cn5)cc4=O)ccc3c2C1]

A mixture of 5-((5-fluoropyridin-2-yl)methoxy)pyridazin-3(2H)-one (93 mg, 0.42 mmol), tert-butyl 8-bromo-4,5-dihydro-1H-benzofuro[3,2-c]azepine-2(3H)-carboxylate (128 mg, 0.349 mmol) and Cs₂CO₃ (148 mg, 0.454 mmol) in toluene (5 mL) in a sealed tube was degassed with a nitrogen stream for 10 min. Trans-N,N′-dimethylcyclohexane-1,2-diamine (75 mg, 0.53 mmol) and CuI (100 mg, 0.53 mmol) were added, and the mixture was degassed for another 2 min. The tube was sealed, and the mixture was heated at 110° C. for 12 h. The mixture was cooled, diluted with 90:9:1 CH₂Cl₂/MeOH/NH₄OH (15 mL) and stirred for 1 h. Then 2:1 brine/NH₄OH (100 mL) was added, and the aqueous phase was extracted with dichloromethane (4×75 mL). The combined organic extracts were washed with 2:1 brine/NH₄OH (3×75 mL), dried over Na₂SO₄ and concentrated under reduced pressure. Purification by flash chromatography (silica gel, CH₂Cl₂/(90:9:1 CH₂Cl₂/MeOH/NH₄OH), 100:0 to 25:75) afforded the title compound (102 mg, 58%) as an off-white solid: ESI MS m/z 507 [M+H]⁺.

### b) 5-((5-Fluoropyridin-2-yl)methoxy)-2-(2,3,4,5-tetrahydro-1H-benzofuro[3,2-c]azepin-8-yl)pyridazin-3(2H)-one

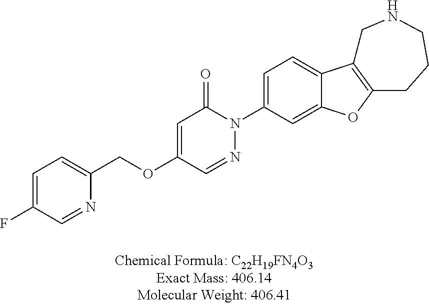 [SMILES: O=c1cc(OCc2ccc(F)cn2)cnn1-c1ccc2c3c(oc2c1)CCCNC3]

A solution of tert-butyl 8-(4-((5-fluoropyridin-2-yl)methoxy)-6-oxopyridazin-1(6H)-yl)-4,5-dihydro-1H-benzofuro[3,2-c]azepine-2(3H)-carboxylate (96 mg, 0.19 mmol) in MeOH (4 mL) was treated with 2 N HCl in Et₂O (2 mL), and the resulting solution was stirred at ambient temperature for 12 h. Additional 2 N HCl in Et₂O (0.2 mL) was added, and the resulting solution was stirred for another 4 h. Saturated aqueous NaHCO₃ (50 mL) was added, and the mixture was extracted with dichloromethane (8×50 mL). The combined organic extracts were dried over Na₂SO₄ and concentrated under reduced pressure. Purification by flash chromatography (silica gel, CH₂Cl₂/(90:9:1 CH₂Cl₂/MeOH/NH₄OH), 100:0 to 10:90) afforded the title compound (65 mg, 84%) as a white solid: ESI MS m/z 407 [M+H]⁺.

### c) 5-((5-Fluoropyridin-2-yl)methoxy)-2-(2,3,4,5-tetrahydro-1H-benzofuro[3,2-c]azepin-8-yl)pyridazin-3(2H)-one hydrochloride

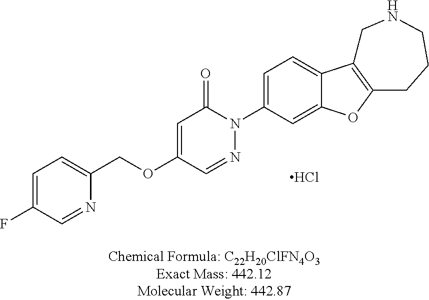 [SMILES: O=c1cc(OCc2ccc(F)cn2)cnn1-c1ccc2c3c(oc2c1)CCCNC3 ; Cl]

5-((5-Fluoropyridin-2-yl)methoxy)-2-(2,3,4,5-tetrahydro-1H-benzofuro[3,2-c]azepin-8-yl)pyridazin-3(2H)-one (60 mg, 0.15 mmol) was suspended in MeOH (3 mL) and treated with 2 N HCl in Et₂O (74 μL, 0.15 mmol). The suspension was stirred at ambient temperature for 2 h, concentrated and lyophilized from acetonitrile-water to provide the title compound (68 mg, quant) as a white solid: ¹H NMR (500 MHz, DMSO-d₆) δ 9.37 (s, 2H), 8.63 (d, J=3.0 Hz, 1H), 8.02 (d, J=3.0 Hz, 1H), 7.88-7.82 (m, 1H), 7.76 (d, J=8.5 Hz, 1H), 7.72-7.68 (m, 2H), 7.40 (dd, J=8.5, 2.0 Hz, 1H), 6.65 (d, J=2.5 Hz, 1H), 5.30 (s, 2H), 4.40 (s, 2H), 3.48-3.45 (m, 2H), 3.11 (t, J=6.0 Hz, 2H), 2.15-2.05 (m, 2H); ESI MS m/z 407 [M+H]⁺; HPLC (Method B)>99% (AUC), t_{R}=12.6 min.

### Example 45

### Preparation of 1-(2,3,4,5-Tetrahydro-1H-benzofuro[3,2-c]azepin-8-yl)-4-((6-(trifluoromethyl)pyridin-3-yl)methoxy)pyridin-2(1H)-one hydrochloride

### a) tert-Butyl 8-(2-oxo-4-((6-(trifluoromethyl)pyridin-3-yl)methoxy)pyridin-1(2H)-yl)-4,5-dihydro-1H-benzofuro[3,2-c]azepine-2(3H)-carboxylate

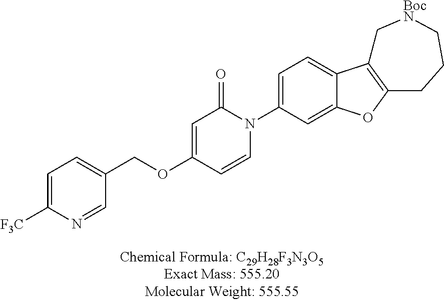 [SMILES: CC(C)(C)OC(=O)N1CCCc2oc3cc(-n4ccc(OCc5ccc(C(F)(F)F)nc5)cc4=O)ccc3c2C1]

A mixture of 4-((6-(trifluoromethyl)pyridin-3-yl)methoxy)pyridin-2(1H)-one (114 mg, 0.422 mmol), tert-butyl 8-bromo-4,5-dihydro-1H-benzofuro[3,2-c]azepine-2(3H)-carboxylate (128 mg, 0.349 mmol) and Cs₂CO₃ (148 mg, 0.454 mmol) in toluene (5 mL) in a sealed tube was degassed with a nitrogen stream for 10 min. Trans-N,N′-dimethylcyclohexane-1,2-diamine (75 mg, 0.53 mmol) and CuI (100 mg, 0.53 mmol) were added, and the mixture was degassed for another 2 min. The tube was sealed, and the mixture was heated at 110° C. for 12 h. The mixture was cooled, diluted with 90:9:1 CH₂Cl₂/MeOH/NH₄OH (15 mL) and stirred for 1 h. Then 2:1 brine/NH₄OH (100 mL) added, and the aqueous phase was extracted with dichloromethane (4×75 mL). The combined organic extracts were washed with 2:1 brine/NH₄OH (3×75 mL), dried over Na₂SO₄ and concentrated under reduced pressure. Purification by flash chromatography (silica gel, CH₂Cl₂/(90:9:1 CH₂Cl₂/MeOH/NH₄OH), 100:0 to 25:75) afforded the title compound (186 mg, 96%) as a white solid: ESI MS m/z 556 [M+H]⁺.

### b) 1-(2,3,4,5-Tetrahydro-1H-benzofuro[3,2-c]azepin-8-yl)-4-((6-(trifluoromethyl)pyridin-3-yl)methoxy)pyridin-2(1H)-one

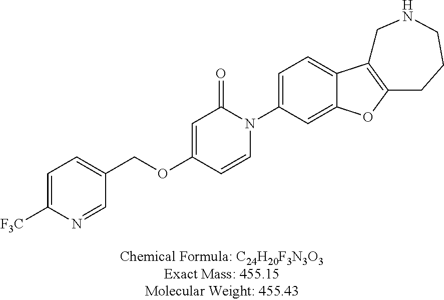 [SMILES: O=c1cc(OCc2ccc(C(F)(F)F)nc2)ccn1-c1ccc2c3c(oc2c1)CCCNC3]

A solution of tert-butyl 8-(2-oxo-4-((6-(trifluoromethyl)pyridin-3-yl)methoxy)pyridin-1(2H)-yl)-4,5-dihydro-1H-benzofuro[3,2-c]azepine-2(3H)-carboxylate (180 mg, 0.32 mmol) in MeOH (5 mL) was treated with 2 N HCl in Et₂O (2.5 mL), and the resulting solution was stirred at ambient temperature for 12 h. Additional 2 N HCl in Et₂O (0.2 mL) was added, and the resulting solution was stirred for another 4 h. Saturated aqueous NaHCO₃ (50 mL) was added, and the mixture was extracted with dichloromethane (8×50 mL). The combined organic extracts were dried over Na₂SO₄ and concentrated under reduced pressure. Purification by flash chromatography (silica gel, CH₂Cl₂/(90:9:1 CH₂Cl₂/MeOH/NH₄OH), 100:0 to 10:90) afforded the title compound (133 mg, 90%) as a white solid: ¹H NMR (500 MHz, DMSO-d₆) δ 8.89 (s, 1H), 8.18 (d, J=8.0 Hz, 1H), 7.99 (d, J=8.0 Hz, 1H), 7.61 (d, J=8.0 Hz, 1H), 7.54 (d, J=8.0 Hz, 1H), 7.49 (d, J=1.5 Hz, 1H), 7.15 (dd, J=8.0, 2.0 Hz, 1H), 6.14 (dd, J=8.0, 3.0 Hz, 1H), 6.02 (d, J=2.5 Hz, 1H), 5.34 (s, 2H), 3.87 (s, 2H), 3.03-2.97 (m, 4H), 1.83-1.80 (m, 2H); ESI MS m/z 456 [M+H]⁺.

### c) 1-(2,3,4,5-Tetrahydro-1H-benzofuro[3,2-c]azepin-8-yl)-4-((6-(trifluoromethyl)pyridin-3-yl)methoxy)pyridin-2(1H)-one hydrochloride

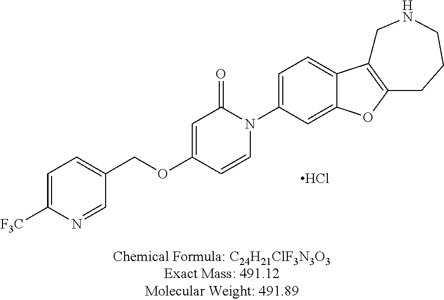 [SMILES: O=c1cc(OCc2ccc(C(F)(F)F)nc2)ccn1-c1ccc2c3c(oc2c1)CCCNC3 ; Cl]

1-(2,3,4,5-Tetrahydro-1H-benzofuro[3,2-c]azepin-8-yl)-4-((6-(trifluoromethyl)pyridin-3-yl)methoxy)pyridin-2(1H)-one (130 mg, 0.29 mmol) was suspended in MeOH (5 mL) and treated with 2 N HCl in Et₂O (143 μL, 0.286 mmol). The suspension was stirred at ambient temperature for 2 h, concentrated and lyophilized from acetonitrile-water to provide the title compound (136 mg, 97%) as a white solid: ¹H NMR (500 MHz, DMSO-d₆) δ 9.42 (s, 2H), 8.89 (s, 1H), 8.20-8.18 (m, 1H), 7.99 (d, J=8.5 Hz, 1H), 7.76 (d, J=8.0 Hz, 1H), 7.64-7.62 (m, 2H), 7.26-7.24 (m, 1H), 6.18-6.16 (m, 1H), 6.03 (d, J=2.5 Hz, 1H), 5.35 (s, 2H), 4.40 (s, 2H), 3.48-3.46 (m, 2H), 3.11 (t, J=5.5 Hz, 2H), 2.10-2.08 (m, 2H); ESI MS m/z 456 [M+H]⁺; HPLC (Method B)>99% (AUC), t_{R}=13.8 min.

### Example 46

### Preparation of 4-((6-Methylpyridin-3-yl)methoxy)-1-(2,3,4,5-tetrahydro-1H-benzofuro[3,2-c]azepin-8-yl)pyridin-2(1H)-one hydrochloride

### a) tert-Butyl 8-(4-((6-methylpyridin-3-yl)methoxy)-2-oxopyridin-1(2H)-yl)-4,5-dihydro-1H-benzofuro[3,2-c]azepine-2(3H)-carboxylate

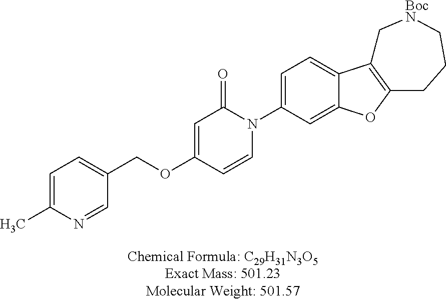 [SMILES: Cc1ccc(COc2ccn(-c3ccc4c5c(oc4c3)CCCN(C(=O)OC(C)(C)C)C5)c(=O)c2)cn1]

A mixture of 4-((6-methylpyridin-3-yl)methoxy)pyridin-2(1H)-one (91 mg, 0.42 mmol), tert-butyl 8-bromo-4,5-dihydro-1H-benzofuro[3,2-c]azepine-2(3H)-carboxylate (128 mg, 0.349 mmol) and Cs₂CO₃ (148 mg, 0.454 mmol) in toluene (5 mL) in a sealed tube was degassed with a nitrogen stream for 10 min. Trans-N,N′-dimethylcyclohexane-1,2-diamine (75 mg, 0.53 mmol) and CuI (100 mg, 0.53 mmol) were added, and the mixture was degassed for another 2 min. The tube was sealed, and the mixture was heated at 110° C. for 12 h. The mixture was cooled, diluted with 90:9:1 CH₂Cl₂/MeOH/NH₄OH (15 mL) and stirred for 1 h. Then 2:1 brine/NH₄OH (100 mL) was added, and the aqueous phase was extracted with dichloromethane (4×75 mL). The combined organic extracts were washed with 2:1 brine/NH₄OH (3×75 mL), dried over Na₂SO₄ and concentrated under reduced pressure. Purification by flash chromatography (silica gel, CH₂Cl₂/(90:9:1 CH₂Cl₂/MeOH/NH₄OH), 100:0 to 25:75) afforded the title compound (170 mg, 97%) as a white solid: ESI MS m/z 502 [M+H]⁺.

### b) 4-((6-Methylpyridin-3-yl)methoxy)-1-(2,3,4,5-tetrahydro-1H-benzofuro[3,2-c]azepin-8-yl)pyridin-2(1H)-one

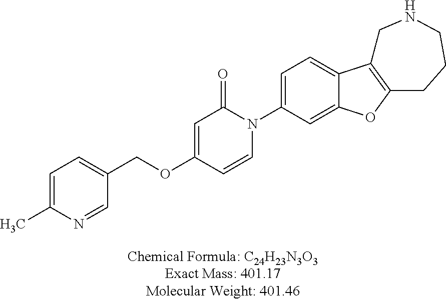 [SMILES: Cc1ccc(COc2ccn(-c3ccc4c5c(oc4c3)CCCNC5)c(=O)c2)cn1]

A solution of tert-butyl 8-(4-((6-methylpyridin-3-yl)methoxy)-2-oxopyridin-1(2H)-yl)-4,5-dihydro-1H-benzofuro[3,2-c]azepine-2(3H)-carboxylate (164 mg, 0.327 mmol) in MeOH (4 mL) was treated with 2 N HCl in Et₂O (2 mL), and the resulting solution was stirred at ambient temperature for 12 h. Saturated aqueous NaHCO₃ (40 mL) was added, and the mixture was extracted with dichloromethane (8×40 mL). The combined organic extracts were dried over Na₂SO₄ and concentrated under reduced pressure. Purification by flash chromatography (silica gel, CH₂Cl₂/(90:9:1 CH₂Cl₂/MeOH/NH₄OH), 100:0 to 10:90) afforded the title compound (110 mg, 84%) as a white solid: ESI MS m/z 402 [M+H]⁺.

### c) 4-((6-Methylpyridin-3-yl)methoxy)-1-(2,3,4,5-tetrahydro-1H-benzofuro[3,2-c]azepin-8-yl)pyridin-2(1H)-one hydrochloride

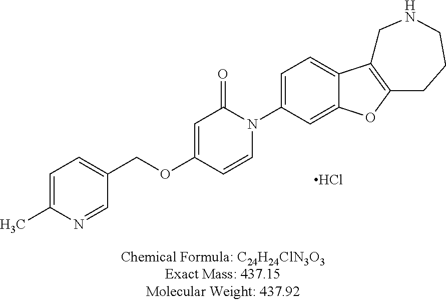 [SMILES: Cc1ccc(COc2ccn(-c3ccc4c5c(oc4c3)CCCNC5)c(=O)c2)cn1 ; Cl]

4-((6-Methylpyridin-3-yl)methoxy)-1-(2,3,4,5-tetrahydro-1H-benzofuro[3,2-c]azepin-8-yl)pyridin-2(1H)-one (107 mg, 0.267 mmol) was suspended in MeOH (4 mL) and treated with 2 N HCl in Et₂O (133 μL, 0.266 mmol). The suspension was stirred at ambient temperature for 1 h. The solvent was removed, and the residue was co-evaporated with acetonitrile and lyophilized from acetonitrile-water to provide the title compound (114 mg, 98%) as a white solid: ¹H NMR (500 MHz, DMSO-d₆) δ 9.33 (s, 2H), 8.63 (s, 1H), 7.94 (s, 1H), 7.76 (d, J=8.5 Hz, 1H), 7.62-7.59 (m, 2H), 7.46 (s, 1H), 7.25 (dd, J=8.5, 1.5 Hz, 1H), 6.13-6.11 (m, 1H), 6.02 (d, J=3.0 Hz, 1H), 5.20 (S, 2H), 4.41 (s, 2H), 3.47 (s, 2H), 3.11 (t, J=5.5 Hz, 2H), 2.55 (s, 3H), 2.09 (s, 2H); ESI MS m/z 402 [M+H]⁺; HPLC (Method B)>99% (AUC), t_{R}=10.0 min.

### Example 47

### Preparation of 1-(2,3,4,5-Tetrahydro-1H-benzofuro[2,3-d]azepin-8-yl)-4-((6-(trifluoromethyl)pyridin-3-yl)methoxy)pyridin-2(1H)-one hydrochloride

### a) tert-Butyl 8-(2-oxo-4-((6-(trifluoromethyl)pyridin-3-yl)methoxy)pyridin-1(2H)-yl)-4,5-dihydro-1H-benzofuro[2,3-d]azepine-3(2H)-carboxylate

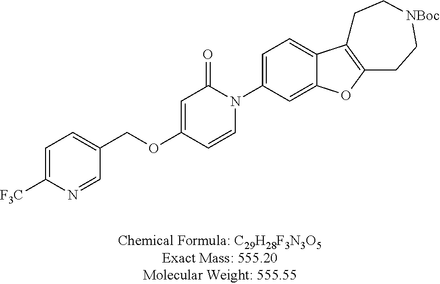 [SMILES: CC(C)(C)OC(=O)N1CCc2oc3cc(-n4ccc(OCc5ccc(C(F)(F)F)nc5)cc4=O)ccc3c2CC1]

A mixture of 4-((6-(trifluoromethyl)pyridin-3-yl)methoxy)pyridin-2(1H)-one (170 mg, 0.63 mmol), tert-butyl 8-bromo-4,5-dihydro-1H-benzofuro[2,3-d]azepine-3(2H)-carboxylate (192 mg, 0.524 mmol) and Cs₂CO₃ (222 mg, 0.681 mmol) in toluene (6 mL) in a sealed tube was degassed with a nitrogen stream for 10 min. Trans-N,N′-dimethylcyclohexane-1,2-diamine (112 mg, 0.787 mmol) and CuI (150 mg, 0.79 mmol) were added, and the mixture was degassed for another 2 min. The tube was sealed and the mixture was heated at 110° C. for 12 h. The mixture was cooled, diluted with 90:9:1 CH₂Cl₂/MeOH/NH₄OH (15 mL) and stirred for 1 h. Then 2:1 brine/NH₄OH (100 mL) was added, and the aqueous phase was extracted with dichloromethane (4×75 mL). The combined organic extracts were washed with 2:1 brine/NH₄OH (3×75 mL), dried over Na₂SO₄ and concentrated under reduced pressure. Purification by flash chromatography (silica gel, CH₂Cl₂/(90:9:1 CH₂Cl₂/MeOH/NH₄OH), 100:0 to 25:75) afforded the title compound (141 mg, 48%) as an off-white solid: ESI MS m/z 556 [M+H]⁺.

### b) 1-(2,3,4,5-Tetrahydro-1H-benzofuro[2,3-d]azepin-8-yl)-4-((6-(trifluoromethyl)pyridin-3-yl)methoxy)pyridin-2(1H)-one

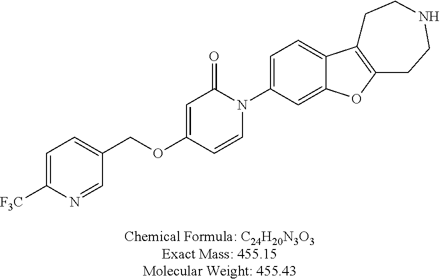 [SMILES: O=c1cc(OCc2ccc(C(F)(F)F)nc2)ccn1-c1ccc2c3c(oc2c1)CCNCC3]

A solution of tert-butyl 8-(2-oxo-4-((6-(trifluoromethyl)pyridin-3-yl)methoxy)pyridin-1(2H)-yl)-4,5-dihydro-1H-benzofuro[2,3-d]azepine-3(2H)-carboxylate (135 mg, 0.243 mmol) in MeOH (3 mL) was treated with 2 N HCl in Et₂O (2.5 mL), and the resulting solution was stirred at ambient temperature for 12 h. Saturated aqueous NaHCO₃ (40 mL) was added, and the mixture was extracted with dichloromethane (8×40 mL). The combined organic extracts were dried over Na₂SO₄ and concentrated under reduced pressure. Purification by flash chromatography (silica gel, CH₂Cl₂/(90:9:1 CH₂Cl₂/MeOH/NH₄OH), 100:0 to 10:90) afforded the title compound (86 mg, 78%) as a white solid: ESI MS m/z 456 [M+H]⁺.

### c) 1-(2,3,4,5-Tetrahydro-1H-benzofuro[2,3-d]azepin-8-yl)-4-((6-(trifluoromethyl)pyridin-3-yl)methoxy)pyridin-2(1H)-one hydrochloride

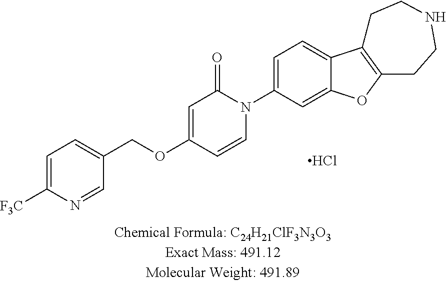 [SMILES: O=c1cc(OCc2ccc(C(F)(F)F)nc2)ccn1-c1ccc2c3c(oc2c1)CCNCC3 ; Cl]

1-(2,3,4,5-Tetrahydro-1H-benzofuro[2,3-d]azepin-8-yl)-4-((6-(trifluoromethyl)pyridin-3-yl)methoxy)pyridin-2(1H)-one (84 mg, 0.18 mmol) was suspended in MeOH (3 mL) and treated with 2 N HCl in Et₂O (92 μL, 0.18 mmol). The suspension was stirred at ambient temperature for 30 min. The solvent was removed, and the residue was co-evaporated with acetonitrile and lyophilized from acetonitrile-water to provide the title compound (93 mg, quant) as a white solid: ¹H NMR (500 MHz, DMSO-d₆) δ 9.41 (s, 2H), 8.89 (s, 1H), 8.20-8.18 (m, 1H), 7.99 (d, J=8.0 Hz, 1H), 7.65-7.59 (m, 3H), 7.24-7.22 (m, 1H), 6.17-6.15 (m, 1H), 6.03 (d, J=2.5 Hz, 1H), 5.35 (s, 2H), 3.40 (t, J=5.5 Hz, 4H), 3.30-3.28 (m, 2H), 3.08 (t, J=5.5 Hz, 2H); ESI MS m/z 456 [M+H]⁺; HPLC (Method B)>99% (AUC), t_{R}=13.9 min.

### Example 48

### Preparation of 1-(2,3,4,5-Tetrahydro-1H-benzofuro[3,2-c]azepin-8-yl)-4-(4-(trifluoromethyl)phenyl)pyridin-2(1H)-one hydrochloride

### a) tert-Butyl 8-(2-oxo-4-(4-(trifluoromethyl)phenyl)pyridin-1(2H)-yl)-4,5-dihydro-1H-benzofuro[3,2-c]azepine-2(3H)-carboxylate

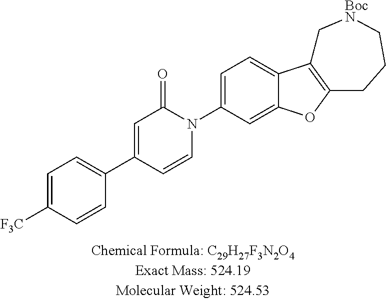 [SMILES: CC(C)(C)OC(=O)N1CCCc2oc3cc(-n4ccc(-c5ccc(C(F)(F)F)cc5)cc4=O)ccc3c2C1]

A mixture of 4-(4-(trifluoromethyl)phenyl)pyridin-2(1H)-one (100 mg, 0.42 mmol), tert-butyl 8-bromo-4,5-dihydro-1H-benzofuro[3,2-c]azepine-2(3H)-carboxylate (128 mg, 0.349 mmol) and Cs₂CO₃ (148 mg, 0.454 mmol) in toluene (5 mL) in a sealed tube was degassed with a nitrogen stream for 10 min. Trans-N,N′-dimethylcyclohexane-1,2-diamine (75 mg, 0.53 mmol) and CuI (100 mg, 0.53 mmol) were added, and the mixture was degassed for another 2 min. The tube was sealed, and the mixture was heated at 110° C. for 12 h. The mixture was cooled, diluted with 90:9:1 CH₂Cl₂/MeOH/NH₄OH (15 mL) and stirred for 1 h. Then 2:1 brine/NH₄OH (100 mL) was added, and the aqueous phase was extracted with dichloromethane (4×75 mL). The combined organic extracts were washed with 2:1 brine/NH₄OH (3×75 mL), dried over Na₂SO₄ and concentrated under reduced pressure. Purification by flash chromatography (silica gel, CH₂Cl₂/(90:9:1 CH₂Cl₂/MeOH/NH₄OH), 100:0 to 25:75) afforded the title compound (159 mg, 87%) as an off-white solid: ESI MS m/z 525 [M+H]⁺.

### b) 1-(2,3,4,5-Tetrahydro-1H-benzofuro[3,2-c]azepin-8-yl)-4-(4-(trifluoromethyl)phenyl)pyridin-2(1H)-one

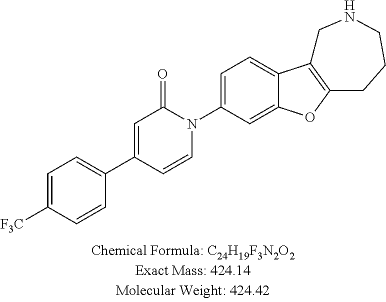 [SMILES: O=c1cc(-c2ccc(C(F)(F)F)cc2)ccn1-c1ccc2c3c(oc2c1)CCCNC3]

A solution of tert-butyl 8-(2-oxo-4-(4-(trifluoromethyl)phenyl)pyridin-1(2H)-yl)-4,5-dihydro-1H-benzofuro[3,2-c]azepine-2(3H)-carboxylate (155 mg, 0.296 mmol) in MeOH (4 mL) was treated with 2 N HCl in Et₂O (3 mL), and the resulting solution was stirred at ambient temperature for 12 h. Saturated aqueous NaHCO₃ (40 mL) was added, and the mixture was extracted with dichloromethane (8×40 mL). The combined organic extracts were dried over Na₂SO₄ and concentrated under reduced pressure. Purification by flash chromatography (silica gel, CH₂Cl₂/(90:9:1 CH₂Cl₂/MeOH/NH₄OH), 100:0 to 10:90) afforded the title compound (110 mg, 88%) as a white solid: ESI MS m/z 425 [M+H]⁺.

### c) 1-(2,3,4,5-Tetrahydro-1H-benzofuro[3,2-c]azepin-8-yl)-4-(4-(trifluoromethyl)phenyl)pyridin-2(1H)-one hydrochloride

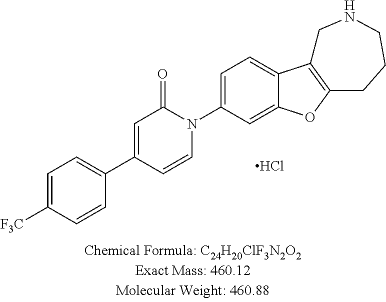 [SMILES: O=c1cc(-c2ccc(C(F)(F)F)cc2)ccn1-c1ccc2c3c(oc2c1)CCCNC3 ; Cl]

1-(2,3,4,5-Tetrahydro-1H-benzofuro[3,2-c]azepin-8-yl)-4-(4-(trifluoromethyl)phenyl)pyridin-2(1H)-one (108 mg, 0.254 mmol) was suspended in MeOH (4 mL) and treated with 2 N HCl in Et₂O (127 μL, 0.254 mmol). The suspension was stirred at ambient temperature for 30 min. The solvent was removed, and the residue was co-evaporated with acetonitrile and lyophilized from acetonitrile-water to provide the title compound (121 mg, quant) as a white solid: ¹H NMR (500 MHz, DMSO-d₆) δ 9.36 (s, 2H), 8.02-8.00 (m, 2H), 7.89-7.87 (m, 2H), 7.82 (t, J=8.0 Hz, 2H), 7.74 (d, J=1.5 Hz, 1H), 7.37-7.35 (m, 1H), 6.88 (d, J=2.0 Hz, 1H), 6.76-6.74 (m, 1H), 4.42 (s, 2H), 3.49-3.47 (m, 2H), 3.13 (t, J=6.0 Hz, 2H), 2.10 (s, 2H); ESI MS m/z 425 [M+H]⁺; HPLC (Method B)>99% (AUC), t_{R}=14.9 min.

### Example 49

### Preparation of 1-(2-Methyl-2,3,4,5-tetrahydro-1H-benzofuro[3,2-c]azepin-8-yl)-4-((6-(trifluoromethyl)pyridin-2-yl)methoxy)pyridin-2(1H)-one hydrochloride

### a) 1-(2-Methyl-2,3,4,5-tetrahydro-1H-benzofuro[3,2-c]azepin-8-yl)-4-((6-(trifluoromethyl)pyridin-2-yl)methoxy)pyridin-2(1H)-one

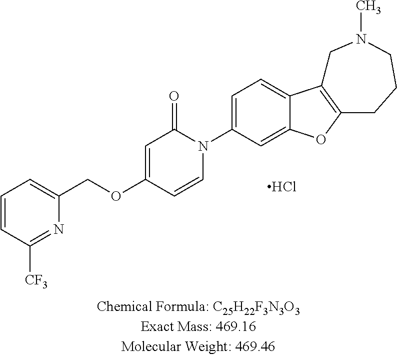 [SMILES: CN1CCCc2oc3cc(-n4ccc(OCc5cccc(C(F)(F)F)n5)cc4=O)ccc3c2C1 ; Cl]

A mixture of 1-(2,3,4,5-tetrahydro-1H-benzofuro[3,2-c]azepin-8-yl)-4-((6-(trifluoromethyl)pyridin-2-yl)methoxy)pyridin-2(1H)-one hydrochloride (49 mg, 0.10 mmol) in dichloromethane (0.6 mL) and methanol (0.6 mL) was treated with 37% aqueous formaldehyde solution (20 μL, 0.25 mmol) followed by sodium triacetoxyborohydride (64 mg, 0.30 mmol). After stirring at ambient temperature for 1 h, the mixture was diluted with saturated aqueous NaHCO₃ solution (50 mL) and extracted with dichloromethane (5×50 mL). The combined organic extracts were dried over Na₂SO₄ and concentrated under reduced pressure. Purification by flash chromatography (silica gel, CH₂Cl₂/(90:9:1 CH₂Cl₂/MeOH/NH₄OH), 100:0 to 25:75) afforded the title compound (43 mg, 92%) as a white solid: ESI MS m/z 470 [M+H]⁺.

### b) 1-(2-Methyl-2,3,4,5-tetrahydro-1H-benzofuro[3,2-c]azepin-8-yl)-4-((6-(trifluoromethyl)pyridin-2-yl)methoxy)pyridin-2(1H)-one hydrochloride

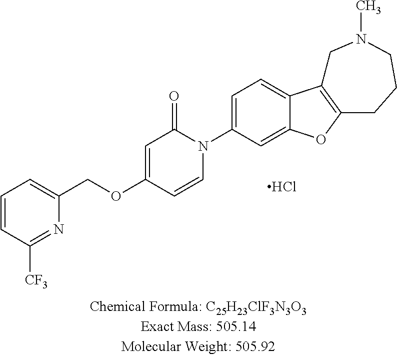 [SMILES: CN1CCCc2oc3cc(-n4ccc(OCc5cccc(C(F)(F)F)n5)cc4=O)ccc3c2C1 ; Cl]

1-(2-Methyl-2,3,4,5-tetrahydro-1H-benzofuro[3,2-c]azepin-8-yl)-4-((6-(trifluoromethyl)pyridin-2-yl)methoxy)pyridin-2(1H)-one (43 mg, 0.092 mmol) was suspended in MeOH (2 mL) and treated with 2 N HCl in Et₂O (46 μL, 0.092 mmol). The suspension was stirred at ambient temperature for 5 min. The solvent was removed, and the residue was co-evaporated with acetonitrile, triturated (acetonitrile) and lyophilized from acetonitrile-water to provide the title compound (26 mg, 57%) as a white solid: ¹H NMR (500 MHz, DMSO-d₆) δ 10.46 (s, 1H), 8.21 (t. J=8.0 Hz, 1H), 7.93 (d, J=8.0 Hz, 1H), 7.89 (d, J=8.0 Hz, 1H), 7.73 (d, J=8.0 Hz, 1H), 7.65-7.62 (m, 2H), 7.28 (dd, J=8.0, 2.0 Hz, 1H), 6.22-6.18 (m, 1H), 6.00 (d, J=3.0 Hz, 1H), 5.33 (s, 2H), 4.69-4.48 (m, 2H), 3.68-3.50 (m, 2H), 3.10 (t, J=5.5 Hz, 2H), 2.90 (s, 3H), 2.27-2.05 (m, 2H); ESI MS m/z 470 [M+H]⁺; HPLC (Method B)>99% (AUC), t_{R}=14.2 min.

### Example 50

### Preparation of 5-((5-Fluoropyridin-2-yl)methoxy)-2-(1,2,3,4-tetrahydrobenzofuro[3,2-c]pyridin-7-yl)pyridazin-3(2H)-one hydrochloride

### a) 5-((5-Fluoropyridin-2-yl)methoxy)pyridazin-3(2H)-one

CAS Registry Number 1008518-12-4

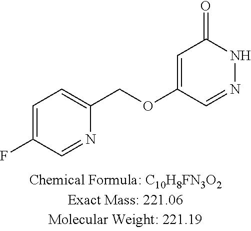 [SMILES: O=c1cc(OCc2ccc(F)cn2)cn[nH]1]

This compound was prepared in accordance with the procedure described in PCT Publication No. WO 2011/003005 to Guzzo et al., which is hereby incorporated by reference in its entirety.

### b) tert-Butyl 7-(4-((5-fluoropyridin-2-yl)methoxy)-6-oxopyridazin-1(6H)-yl)-3,4-dihydrobenzofuro[3,2-c]pyridine-2(1H)-carboxylate

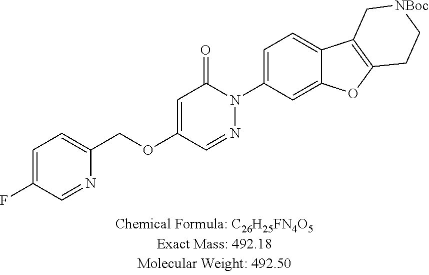 [SMILES: CC(C)(C)OC(=O)N1CCc2oc3cc(-n4ncc(OCc5ccc(F)cn5)cc4=O)ccc3c2C1]

tert-Butyl 7-bromo-3,4-dihydrobenzofuro[3,2-c]pyridine-2(1H)-carboxylate (205 mg, 0.582 mmol) and 5-((5-fluoropyridin-2-yl)methoxy)pyridazin-3(2H)-one (128 mg, 0.579 mmol) were reacted according to Example 3 (step b) to provide the title compound (225 mg, 79%) as a white solid: ¹H NMR (500 MHz, CDCl₃) δ 8.50 (t, J=1.5 Hz, 1H), 7.81 (d, J=3.0 Hz, 1H), 7.65 (d, J=2.0 Hz, 1H), 7.50 (dd, J=6.5, 2.0 Hz, 2H), 7.46 (d, J=8.0 Hz, 1H), 7.41 (d, J=8.5 Hz, 1H), 6.35 (d, J=2.5 Hz, 1H), 5.19 (s, 2H), 4.57 (s, 2H), 3.84 (s, 2H), 2.87 (s, 2H), 1.51 (s, 9H); ESI MS m/z 493 [M+H]⁺.

### c) 5-((5-Fluoropyridin-2-yl)methoxy)-2-(1,2,3,4-tetrahydrobenzofuro[3,2-c]pyridin-7-yl)pyridazin-3(2H)-one

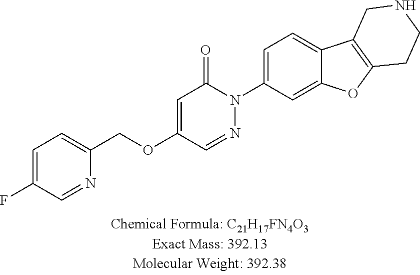 [SMILES: O=c1cc(OCc2ccc(F)cn2)cnn1-c1ccc2c3c(oc2c1)CCNC3]

A solution of tert-butyl 7-(4-((5-fluoropyridin-2-yl)methoxy)-6-oxopyridazin-1(6H)-yl)-3,4-dihydrobenzofuro[3,2-c]pyridine-2(1H)-carboxylate (222 mg, 0.451 mmol) in MeOH (5.0 mL) was treated with 2 N HCl in Et₂O (20 mL), and the resulting solution was stirred at ambient temperature for 16 h and then concentrated in vacuo. The resultant HCl salt was converted to the corresponding free base using an SCX-2 cartridge to provide the title compound (172 mg, 97%) as a white solid: ¹H NMR (500 MHz, CD₃OD) δ 8.52 (d, J=2.5 Hz, 1H), 7.95 (d, J=3.0 Hz, 1H), 7.71-7.68 (m, 2H), 7.60 (d, J=2.0 Hz, 1H), 7.51 (d, J=8.5 Hz, 1H), 7.34 (dd, J=8.3, 1.8 Hz, 1H), 6.50 (d, J=2.5 Hz, 1H), 5.29 (s, 2H), 3.96 (t, J=1.8 Hz, 2H), 3.21 (t, J=5.8 Hz, 2H), 2.85 (t, J=5.8 Hz, 2H); ESI MS m/z 393 [M+H]⁺.

### d) 5-((5-Fluoropyridin-2-yl)methoxy)-2-(1,2,3,4-tetrahydrobenzofuro[3,2-c]pyridin-7-yl)pyridazin-3(2H)-one hydrochloride

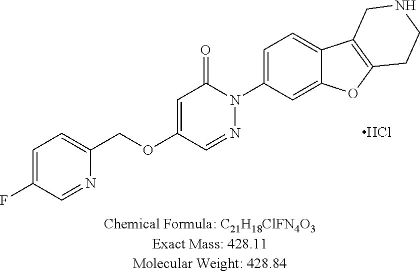 [SMILES: O=c1cc(OCc2ccc(F)cn2)cnn1-c1ccc2c3c(oc2c1)CCNC3 ; Cl]

A solution of 5-((5-fluoropyridin-2-yl)methoxy)-2-(1,2,3,4-tetrahydrobenzofuro[3,2-c]pyridin-7-yl)pyridazin-3(2H)-one (171 mg, 0.436 mmol) in MeOH (5.0 mL) was treated with 2 N HCl in Et₂O (0.22 mL, 0.44 mmol), and the resulting suspension was concentrated and dried under high vac at 55° C. to provide the title compound (182 mg, 96%) as a white solid: mp 268-269° C.; ¹H NMR (500 MHz, DMSO-d₆) δ 9.49 (s, 2H), 8.64 (d, J=3.0 Hz, 1H), 8.03 (d, J=2.5 Hz, 1H), 7.85 (td, J=8.8, 3.0 Hz, 1H), 7.79 (d, J=1.5 Hz, 1H), 7.72-7.68 (m, 2H), 7.43 (dd, J=8.3, 1.8 Hz, 1H), 6.56 (d, J=3.0 Hz, 1H), 5.30 (s, 2H), 4.36 (s, 2H), 3.56 (s, 2H), 3.12 (t, J=5.8 Hz, 2H); ESI MS m/z 393 [M+H]⁺; HPLC (Method A)>99% (AUC), t_{R}=12.4 min.

### Example 51

### Preparation of 4-((6-Methylpyridin-3-yl)methoxy)-1-(1,2,3,4-tetrahydrobenzofuro[3,2-c]pyridin-7-yl)pyridin-2(1H)-one hydrochloride

### a) 4-((6-Methylpyridin-3-yl)methoxy)pyridin-2(1H)-one

CAS Registry Number 1260240-53-6

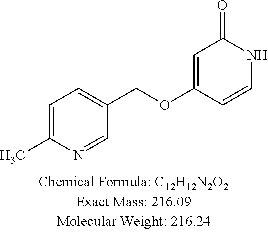 [SMILES: Cc1ccc(COc2cc[nH]c(=O)c2)cn1]

This compound was prepared in accordance with the procedure described in PCT Publication No. WO 2011/003005 to Guzzo et al., which is hereby incorporated by reference in its entirety.

### b) tert-Butyl 7-(4-((6-methylpyridin-3-yl)methoxy)-2-oxopyridin-1(2H)-yl)-3,4-dihydrobenzofuro[3,2-c]pyridine-2(1H)-carboxylate

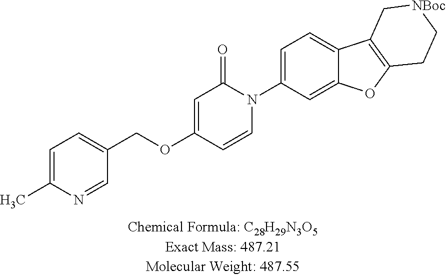 [SMILES: Cc1ccc(COc2ccn(-c3ccc4c5c(oc4c3)CCN(C(=O)OC(C)(C)C)C5)c(=O)c2)cn1]

tert-butyl 7-bromo-3,4-dihydrobenzofuro[3,2-c]pyridine-2(1H)-carboxylate (210 mg, 0.60 mmol) and 4-((6-methylpyridin-3-yl)methoxy)pyridin-2(1H)-one (130 mg, 0.60 mmol) were reacted according to Example 3 (step b) to provide the title compound (269 mg, 92%) as a white solid: ¹H NMR (500 MHz, CDCl₃) δ 8.56 (d, J=2.0 Hz, 1H), 7.65 (dd, J=8.0, 2.0 Hz, 1H), 7.48 (d, J=8.0 Hz, 1H), 7.46 (d, J=1.5 Hz, 1H), 7.28 (d, J=7.5 Hz, 1H), 7.21 (d, J=8.0 Hz, 1H), 7.19 (br s, 1H), 6.07 (d, J=3.0 Hz, 1H), 6.04 (dd, J=7.5, 3.0 Hz, 1H), 5.02 (s, 2H), 4.57 (s, 2H), 3.84 (s, 2H), 2.87 (s, 2H), 2.60 (s, 3H), 1.51 (s, 9H); ESI MS m/z 488 [M+H]⁺.

### c) 4-((6-Methylpyridin-3-yl)methoxy)-1-(1,2,3,4-tetrahydrobenzofuro[3,2-c]pyridin-7-yl)pyridin-2(1H)-one

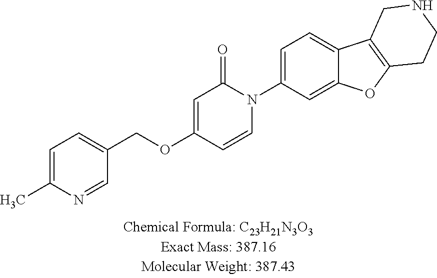 [SMILES: Cc1ccc(COc2ccn(-c3ccc4c5c(oc4c3)CCNC5)c(=O)c2)cn1]

A solution of tert-butyl 7-(4-((6-methylpyridin-3-yl)methoxy)-2-oxopyridin-1(2H)-yl)-3,4-dihydrobenzofuro[3,2-c]pyridine-2(1H)-carboxylate (266 mg, 0.546 mmol) in MeOH (5.0 mL) was treated with 2 N HCl in Et₂O (20 mL), and the resulting solution was stirred at ambient temperature for 16 h and then concentrated in vacuo. The resultant HCl salt was converted to the corresponding free base using an SCX-2 cartridge to provide the title compound (208 mg, 98%) as a white solid: ¹H NMR (500 MHz, CD₃OD) δ 8.52 (d, J=2.0 Hz, 1H), 7.85 (dd, J=8.0, 2.0 Hz, 1H), 7.58 (d, J=7.5 Hz, 1H), 7.55 (d, J=8.0 Hz, 1H), 7.49 (d, J=1.5 Hz, 1H), 7.37 (d, J=8.0 Hz, 1H), 7.19 (dd, J=8.5, 2.0 Hz, 1H), 6.27 (dd, J=7.5, 3.0 Hz, 1H), 6.14 (d, J=3.0 Hz, 1H), 5.19 (s, 2H), 3.96 (t, J=2.0 Hz, 2H), 3.21 (t, J=6.0 Hz, 2H), 2.86-2.84 (m, 2H), 2.56 (s, 3H); ESI MS m/z 388 [M+H]⁺.

### d) 4-((6-Methylpyridin-3-yl)methoxy)-1-(1,2,3,4-tetrahydrobenzofuro[3,2-c]pyridin-7-yl)pyridin-2(1H)-one hydrochloride

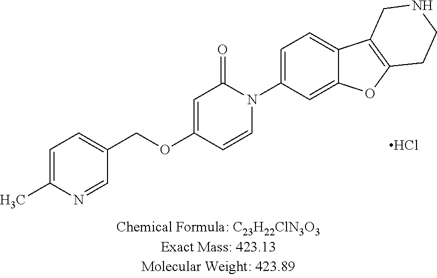 [SMILES: Cc1ccc(COc2ccn(-c3ccc4c5c(oc4c3)CCNC5)c(=O)c2)cn1 ; Cl]

A solution of 4-((6-methylpyridin-3-yl)methoxy)-1-(1,2,3,4-tetrahydrobenzofuro[3,2-c]pyridin-7-yl)pyridin-2(1H)-one (205 mg, 0.529 mmol) in MeOH (5.0 mL) was treated with 2 N HCl in Et₂O (0.27 mL, 0.53 mmol), and the resulting suspension was concentrated. The solid was then suspended in H₂O (3 mL), frozen, and lyophilized overnight to provide the title compound (229 mg, quant. yield) as a white solid: mp 227-233° C.; ¹H NMR (500 MHz, DMSO-d₆) δ 9.68 (s, 2H), 8.69 (s, 1H), 8.06 (d, J=8.0 Hz, 1H), 7.70-7.67 (m, 2H), 7.62 (d, J=8.0 Hz, 1H), 7.56 (d, J=8.0 Hz, 1H), 7.27 (dd, J=8.3, 1.8 Hz, 1H), 6.13 (dd, J=7.5, 3.0 Hz, 1H), 6.03 (d, J=2.5 Hz, 1H), 5.23 (s, 2H), 4.35 (s, 2H), 3.56-3.53 (m, 2H), 3.12 (t, J=5.5 Hz, 2H), 2.60 (s, 3H); ESI MS m/z 388 [M+H]⁺; HPLC (Method A)>99% (AUC), t_{R}=9.7 min.

### Example 52

### Preparation of 1-(1,2,3,4-Tetrahydrobenzofuro[3,2-c]pyridin-7-yl)-4-((6-(trifluoromethyl)pyridin-3-yl)methoxy)pyridin-2(1H)-one hydrochloride

### a) 4-((6-(Trifluoromethyl)pyridin-3-yl)methoxy)pyridin-2(1H)-one

CAS Registry Number 1173155-96-8

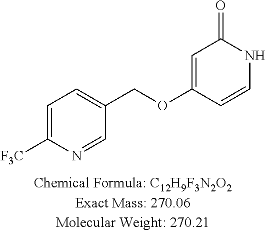 [SMILES: O=c1cc(OCc2ccc(C(F)(F)F)nc2)cc[nH]1]

This compound was prepared in accordance with the procedure described in PCT Publication No. WO 2009/089482 to Guzzo et al., which is hereby incorporated by reference in its entirety.

### b) tert-Butyl 7-(2-oxo-4-((6-(trifluoromethyl)pyridin-3-yl)methoxy)pyridin-1(2H)-yl)-3,4-dihydrobenzofuro[3,2-c]pyridine-2(1H)-carboxylate

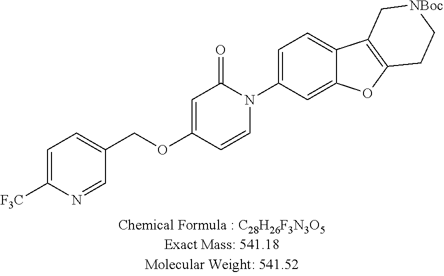 [SMILES: CC(C)(C)OC(=O)N1CCc2oc3cc(-n4ccc(OCc5ccc(C(F)(F)F)nc5)cc4=O)ccc3c2C1]

tert-Butyl 7-bromo-3,4-dihydrobenzofuro[3,2-c]pyridine-2(1H)-carboxylate (184 mg, 0.522 mmol) and 4-((6-(trifluoromethyl)pyridin-3-yl)methoxy)pyridin-2(1H)-one (141 mg, 0.522 mmol) were reacted according to Example 3 (step b) to provide the title compound (232 mg, 82%) as a white solid: ¹H NMR (500 MHz, CDCl₃) δ 8.81 (s, 1H), 7.96 (d, J=8.0 Hz, 1H), 7.75 (d, J=8.5 Hz, 1H), 7.49 (d, J=8.5 Hz, 1H), 7.46 (d, J=2.0 Hz, 1H), 7.32 (d, J=7.5 Hz, 1H), 7.20 (d, J=7.5 Hz, 1H), 6.08-6.05 (m, 2H), 5.16 (s, 2H), 4.57 (s, 2H), 3.84 (s, 2H), 2.88 (s, 2H), 1.51 (s, 9H); ESI MS m/z 542 [M+H]⁺.

### c) 1-(1,2,3,4-Tetrahydrobenzofuro[3,2-c]pyridin-7-yl)-4-((6-(trifluoromethyl)pyridin-3-yl)methoxy)pyridin-2(1H)-one

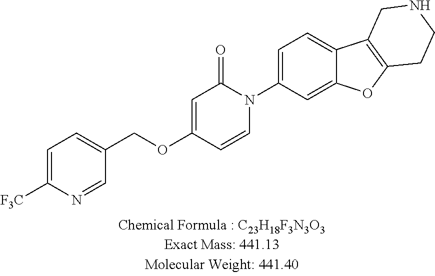 [SMILES: O=c1cc(OCc2ccc(C(F)(F)F)nc2)ccn1-c1ccc2c3c(oc2c1)CCNC3]

A solution of tert-butyl 7-(2-oxo-4-((6-(trifluoromethyl)pyridin-3-yl)methoxy)pyridin-1(2H)-yl)-3,4-dihydrobenzofuro[3,2-c]pyridine-2(1H)-carboxylate (231 mg, 0.427 mmol) in MeOH (5.0 mL) was treated with 2 N HCl in Et₂O (20 mL), and the resulting solution was stirred at ambient temperature for 16 h and then concentrated in vacuo. The resultant HCl salt was converted to the corresponding free base using an SCX-2 cartridge to provide the title compound (172 mg, 91%) as a white solid: ¹H NMR (500 MHz, CD₃OD) δ 8.84 (s, 1H), 8.17 (d, J=8.0 Hz, 1H), 7.88 (d, J=8.0 Hz, 1H), 7.60 (d, J=7.5 Hz, 1H), 7.56 (d, J=8.5 Hz, 1H), 7.50 (d, J=1.5 Hz, 1H), 7.19 (dd, J=8.0, 2.0 Hz, 1H), 6.32 (dd, J=7.5, 2.5 Hz, 1H), 6.15 (d, J=3.0 Hz, 1H), 5.34 (s, 2H), 3.97 (t, J=2.0 Hz, 2H), 3.22 (t, J=6.0 Hz, 2H), 2.86-2.84 (m, 2H); ESI MS m/z 442 [M+H]⁺.

### d) 1-(1,2,3,4-Tetrahydrobenzofuro[3,2-c]pyridin-7-yl)-4-((6-(trifluoromethyl)pyridin-3-yl)methoxy)pyridin-2(1H)-one hydrochloride

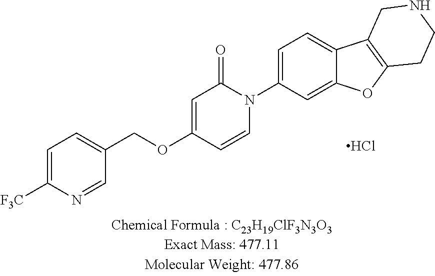 [SMILES: O=c1cc(OCc2ccc(C(F)(F)F)nc2)ccn1-c1ccc2c3c(oc2c1)CCNC3 ; Cl]

A solution of 1-(1,2,3,4-tetrahydrobenzofuro[3,2-c]pyridin-7-yl)-4-((6-(trifluoromethyl)pyridin-3-yl)methoxy)pyridin-2(1H)-one (169 mg, 0.383 mmol) in MeOH (5.0 mL) was treated with 2 N HCl in Et₂O (0.19 mL, 0.38 mmol), and the resulting suspension was concentrated. The solid was then suspended in H₂O (3 mL), frozen, and lyophilized overnight to provide the title compound (182 mg, quant. yield) as a white solid: mp 278-280° C.; ¹H NMR (500 MHz, DMSO-d₆) δ 9.43 (s, 2H), 8.89 (d, J=1.0 Hz, 1H), 8.19 (dd, J=7.8, 1.3 Hz, 1H), 8.00 (d, J=8.0 Hz, 1H), 7.70-7.69 (m, 2H), 7.64 (d, J=7.5 Hz, 1H), 7.28 (dd, J=8.5, 2.0 Hz, 1H), 6.17 (dd, J=7.5, 3.0 Hz, 1H), 6.04 (d, J=2.5 Hz, 1H), 5.35 (s, 2H), 4.36 (s, 2H), 3.56 (s, 2H), 3.12 (t, J=5.8 Hz, 2H); ESI MS m/z 442 [M+H]⁺; HPLC (Method A)>99% (AUC), t_{R}=13.7 min.

### Example 53

### Preparation of 1-(2-Acetyl-1,2,3,4-tetrahydrobenzothieno[2,3-c]pyridin-7-yl)-4-((5-fluoropyridin-2-yl)methoxy)pyridin-2(1H)-one

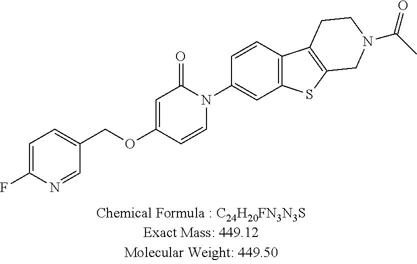 [SMILES: CC(=O)N1CCc2c(sc3cc(-n4ccc(OCc5ccc(F)nc5)cc4=O)ccc23)C1]

4-((5-Fluoropyridin-2-yl)methoxy)-1-(1,2,3,4-tetrahydrobenzothieno[2,3-c]pyridin-7-yl)pyridin-2(1H)-one hydrochloride (117 mg, 0.263 mmol) was stirred in CH₂Cl₂ (5 mL), and triethylamine (206 mg, 2.63 mmol, 0.366 mL) and AcCl (62 mg, 0.78 mmol, 56 μL) were added. After 16 h, the mixture was diluted with CH₂Cl₂ (30 mL) and saturated NaHCO₃ solution (30 mL). The organic layer was removed, dried over sodium sulfate and concentrated. The residue was purified by flash chromatography (silica gel, (CH₂Cl₂/(90:9:1 CH₂Cl₂/MeOH/NH₄OH), 100:0 to 90:10) to provide the title compound (92 mg, 78%) as an off-white solid: ¹H NMR (500 MHz, CDCl₃) δ 8.14 (d, J=2.4 Hz, 1H), 7.79-7.66 (dd, J=4.8, 1.6 Hz, 1H), 7.70-7.64 (m, 1H), 7.50-7.44 (m, 2H), 7.37-7.35 (m, 1H), 7.30 (d, J=7.6 Hz, 1H), 6.12-6.10 (dd, J=7.6, 2.5 Hz, 1H), 6.06 (d, J=2.5 Hz, 1H), 5.17 (s, 2H), 4.90-4.76 (m, 2H), 4.00-3.82 (m, 2H), 2.94-2.87 (m, 2H), 2.24-2.21 (m, 3H); ESI MS m/z 450 [M+H]⁺; HPLC (Method B) 99.2% (AUC), t_{R}=17.9 min.

### Example 54

### Preparation of 7-(4-((5-Fluoropyridin-2-yl)methoxy)-2-oxopyridin-1(2H)-yl)-3,4-dihydrobenzothieno[2,3-c]pyridin-1(2H)-one

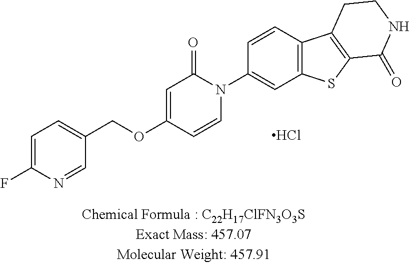 [SMILES: O=C1NCCc2c1sc1cc(-n3ccc(OCc4ccc(F)nc4)cc3=O)ccc21 ; Cl]

The free-base of the title compound was isolated as an impurity from example 14 (step b). The hydrochloride was formed according to Example 12 (step d) to provide the title compound (7 mg, 4%) as a white solid: ¹H NMR (500 MHz, DMSO-d₆) δ 8.62 (d, J=2.9 Hz, 1H), 8.12 (d, J=1.8 Hz, 1H), 8.02-7.97 (m, 2H), 7.84-7.80 (td, J=8.7, 2.9 Hz, 1H), 7.68-7.65 (m, 2H), 7.47-7.45 (dd, J=8.5, 1.9 Hz, 1H), 6.18-6.16 (dd, J=7.6, 2.7 Hz, 1H), 6.01 (d, J=2.7 Hz, 1H), 5.22 (s, 2H), 3.60-3.57 (td, J=7.2, 2.5 Hz, 2H), 3.08 (t, J=7.1 Hz, 2H); ESI MS m/z 422 [M+H]⁺; HPLC (Method B) 99.2% (AUC), t_{R}=17.1 min.

### Example 55

### Preparation of 4-((5-Fluoropyridin-2-yl)methoxy)-1-(1,2,3,4-tetrahydrobenzothieno[3,2-c]pyridin-7-yl)pyridin-2(1H)-one hydrochloride

### a) tert-Butyl 4-(3-bromophenylthio)-3-hydroxypiperidine-1-carboxylate

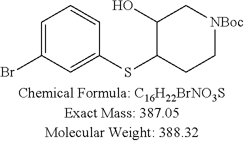 [SMILES: CC(C)(C)OC(=O)N1CCC(Sc2cccc(Br)c2)C(O)C1]

3-Bromothiophenol (12.7 g, 67.2 mmol), NaOH (2.45 g, 61.3 mmol) and tert-butyl 7-oxa-3-azabicyclo[4.1.0]heptane-3-carboxylate (12.2 g, 61.3 mmol) were reacted according to Example 124 (step b) to provide the title compound (12.8 g, 54%) as a yellow oil: ¹H NMR (500 MHz, DMSO-d₆) δ 7.64 (s, 1H), 7.44-7.42 (m, 2H), 7.27 (t, J=7.9 Hz, 1H), 5.44 (d, J=4.8 Hz, 1H), 3.95-3.48 (m, 1H), 3.72-3.64 (m, 1H), 3.28-3.15 (m, 2H), 2.95-2.59 (m, 2H), 1.98-1.90 (m, 1H), 1.40-1.28 (m, 10H).

### b) tert-Butyl 4-(3-bromophenylthio)-3-oxopiperidine-1-carboxylate

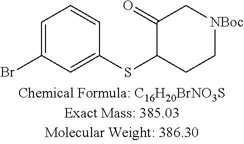 [SMILES: CC(C)(C)OC(=O)N1CCC(Sc2cccc(Br)c2)C(=O)C1]

tert-Butyl 4-(3-bromophenylthio)-3-hydroxypiperidine-1-carboxylate (12.8 g, 33.1 mmol) was reacted according to Example 124 (step c) to provide the title compound (12.5 g, 98%) as a yellow oil: ¹H NMR (500 MHz, CDCl₃) δ 7.58 (d, J=3.3 Hz, 1H), 7.41 (d, J=7.1 Hz, 1H), 7.35 (d, J=7.0 Hz, 1H), 7.18 (t, J=7.9 Hz, 1H), 4.28 (d, J=17.5 Hz, 1H), 4.09 (d, J=17.1 Hz, 1H), 3.84 (t, J=5.8 Hz, 1H), 3.72-3.60 (m, 2H), 2.40-2.31 (m, 1H), 2.17-2.04 (m, 1H), 1.46 (s, 9H).

### c) tert-Butyl 7-bromo-3,4-dihydrobenzothieno[3,2-c]pyridine-2(1H)-carboxylate

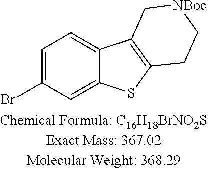 [SMILES: CC(C)(C)OC(=O)N1CCc2sc3cc(Br)ccc3c2C1]

tert-Butyl 4-(3-bromophenylthio)-3-oxopiperidine-1-carboxylate (12.5 g, 32.4 mmol) was reacted according to Example 20 (step a) to provide the title compound (2.69 g, 22%) as a mixture of regioisomers and as a yellow oil: ESI MS m/z 312 [M−t-Bu]⁺.

### d) tert-Butyl 7-(4-((5-fluoropyridin-2-yl)methoxy)-2-oxopyridin-1(2H)-yl)-3,4-dihydrobenzothieno[3,2-c]pyridine-2(1H)-carboxylate

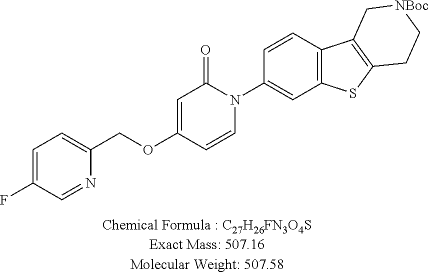 [SMILES: CC(C)(C)OC(=O)N1CCc2sc3cc(-n4ccc(OCc5ccc(F)cn5)cc4=O)ccc3c2C1]

tert-Butyl 7-bromo-3,4-dihydrobenzothieno[3,2-c]pyridine-2(1H)-carboxylate (2.69 g, 7.31 mmol) and 5-((5-fluoropyridin-2-yl)methoxy)pyridazin-3(2H)-one (1.10 g, 5.02 mmol) were reacted according to Example 12 (step c) to provide the title compound (1.30 g, 51%) as a yellow solid: ESI MS m/z 508 [M+H]⁺.

### e) 4-((5-Fluoropyridin-2-yl)methoxy)-1-(1,2,3,4-tetrahydrobenzothieno[3,2-c]pyridin-7-yl)pyridin-2(1H)-one hydrochloride

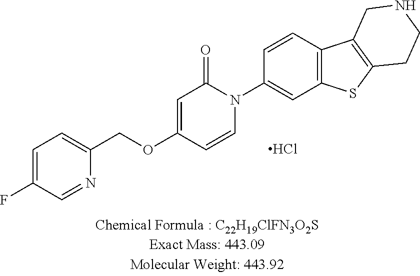 [SMILES: O=c1cc(OCc2ccc(F)cn2)ccn1-c1ccc2c3c(sc2c1)CCNC3 ; Cl]

tert-Butyl 7-(4-((5-fluoropyridin-2-yl)methoxy)-2-oxopyridin-1(2H)-yl)-3,4-dihydrobenzothieno[3,2-c]pyridine-2(1H)-carboxylate (1.30 g, 2.56 mmol) was deprotected and converted to the hydrochloride according to Example 12 (step d) to provide the title compound (1.10 g, 96%) as a white solid: ¹H NMR (500 MHz, DMSO-d₆) δ 9.70 (s, 2H), 8.62 (d, J=2.9 Hz, 1H), 8.04 (d, J=1.9 Hz, 1H), 7.85-7.81 (m, 2H), 7.67-7.62 (m, 2H), 7.40-7.38 (dd, J=8.5, 1.9 Hz, 1H), 6.17-6.16 (dd, J=7.6, 2.7 Hz, 1H), 6.00 (d, J=2.7 Hz, 1H), 5.22 (s, 2H), 4.42 (s, 2H), 3.52-3.47 (m, 2H), 3.21-3.18 (m, 2H); ESI MS m/z 408 [M+H]⁺; HPLC (Method B) 99.2% (AUC), t_{R}=min.

### Example 56

### Preparation of 1-(2-Ethyl-1,2,3,4-tetrahydrobenzothieno[2,3-c]pyridin-7-yl)-4-((5-fluoropyridin-2-yl)methoxy)pyridin-2(1H)-one hydrochloride

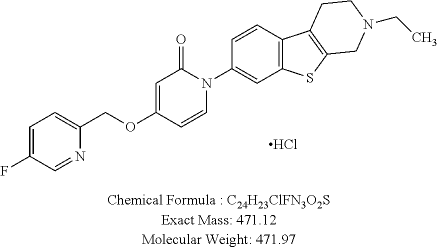 [SMILES: CCN1CCc2c(sc3cc(-n4ccc(OCc5ccc(F)cn5)cc4=O)ccc23)C1 ; Cl]

4-((5-Fluoropyridin-2-yl)methoxy)-1-(1,2,3,4-tetrahydrobenzothieno[2,3-c]pyridin-7-yl)pyridin-2(1H)-one (93 mg, 0.13 mmol) and acetaldehyde (2 mL) were stirred in CH₂Cl₂ (5 mL) and AcOH (0.5 mL), and picoline-borane (55 mg, 0.51 mmol) was added. After 3 h, the mixture was diluted with CH₂Cl₂ (30 mL) and saturated NaHCO₃ solution (30 mL). The organic layer was removed, dried over sodium sulfate and concentrated. The residue was purified by flash chromatography (silica gel, (CH₂Cl₂/(90:9:1 CH₂Cl₂/MeOH/NH₄OH), 100:0 to 90:10). The obtained free-base was converted to the hydrochloride according to Example 12 (step d) to provide the title compound (36 mg, 61%) as a white solid: ¹H NMR (500 MHz, DMSO-d₆) δ 10.9 (s, 1H), 8.62 (d, J=2.9 Hz, 1H), 8.07 (d, J=1.8 Hz, 1H), 7.85-7.79 (m, 2H), 7.68-7.62 (m, 2H), 7.43-7.41 (dd, J=8.4, 1.9 Hz, 1H), 6.18-6.16 (dd, J=7.6, 2.7 Hz, 1H), 6.01 (d, J=2.7 Hz, 1H), 5.22 (s, 2H), 4.77 (d, J=16.1 Hz, 1H), 4.50-4.45 (d, J=16.1, 7.2 Hz, 1H), 3.87-3.82 (m, 1H), 3.40-3.26 (m, 3H), 3.23-3.13 (m, 2H), 1.37 (t, J=7.2 Hz, 3H); ESI MS m/z 436 [M+H]⁺; HPLC (Method B)>99% (AUC), t_{R}=10.5 min.

### Example 57

### Preparation of 4-((5-Fluoropyridin-2-yl)methoxy)-1-(2-isopropyl-1,2,3,4-tetrahydrobenzothieno[2,3-c]pyridin-7-yl)pyridin-2(1H)-one hydrochloride

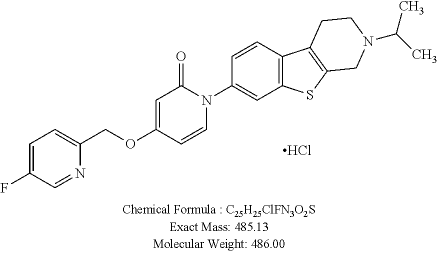 [SMILES: CC(C)N1CCc2c(sc3cc(-n4ccc(OCc5ccc(F)cn5)cc4=O)ccc23)C1 ; Cl]

4-((5-Fluoropyridin-2-yl)methoxy)-1-(1,2,3,4-tetrahydrobenzothieno[2,3-c]pyridin-7-yl)pyridin-2(1H)-one (70 mg, 0.17 mmol) and acetone (5 mL) were stirred at 50° C. in CH₂Cl₂ (5 mL) and AcOH (0.5 mL), and picoline-borane (55 mg, 0.51 mmol) was added. After 72 h, the mixture was allowed to cool, and CH₂Cl₂ (30 mL) and saturated NaHCO₃ solution (30 mL) were added. The mixture was stirred for 1 h. The organic layer was removed, dried over sodium sulfate and concentrated. The residue was purified by flash chromatography (silica gel, (CH₂Cl₂/(90:9:1 CH₂Cl₂/MeOH/NH₄OH), 100:0 to 90:10). The obtained free-base was converted to the hydrochloride according to Example 12 (step d) to provide the title compound (49 mg, 60%) as a white solid: ¹H NMR (500 MHz, DMSO-d₆) δ 10.8 (s, 1H), 8.62 (d, J=2.9 Hz, 1H), 8.06 (d, J=1.9 Hz, 1H), 7.86-7.81 (m, 2H), 7.67-7.63 (m, 2H), 7.43-7.41 (dd, J=8.6, 1.9 Hz, 1H), 6.18-6.16 (dd, J=7.6, 2.7 Hz, 1H), 6.01 (d, J=2.7 Hz, 1H), 5.22 (s, 2H), 4.66-4.62 (dd, J=16.1, 2.1 Hz, 1H), 4.59-4.54 (d, J=16.1, 8.6 Hz, 1H), 3.86-3.79 (m, 1H), 3.74-3.65 (m, 1H), 3.43-3.39 (m, 1H), 3.23-3.18 (m, 2H), 1.39 (t, J=7.2 Hz, 6H); ESI MS m/z 450 [M+H]⁺; HPLC (Method B)>99% (AUC), t_{R}=11.2 min.

### Example 58

### Preparation of 4-((5-Fluoropyridin-2-yl)methoxy)-1-(2-methyl-1,2,3,4-tetrahydrobenzothieno[2,3-c]pyridin-7-yl)pyridin-2(1H)-one hydrochloride

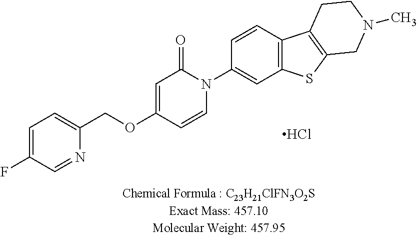 [SMILES: CN1CCc2c(sc3cc(-n4ccc(OCc5ccc(F)cn5)cc4=O)ccc23)C1 ; Cl]

4-((5-Fluoropyridin-2-yl)methoxy)-1-(1,2,3,4-tetrahydrobenzothieno[2,3-c]pyridin-7-yl)pyridin-2(1H)-one (73 mg, 0.18 mmol) and formaldehyde (37% aqueous, 0.5 mL) were stirred in CH₂Cl₂ (3 mL) and MeOH (3 mL), and NaBH(OAc)₃ (76 mg, 0.36 mmol) was added. After 2 h, CH₂Cl₂ (30 mL) and saturated NaHCO₃ solution (30 mL) were added, and the mixture was stirred for 1 h. The organic layer was removed, dried over sodium sulfate and concentrated. The residue was purified by flash chromatography (silica gel, (CH₂Cl₂/(90:9:1 CH₂Cl₂/MeOH/NH₄OH), 100:0 to 90:10). The obtained free-base was converted to the hydrochloride according to Example 12 (step d) to provide the title compound (62 mg, 75%) as a white solid: ¹H NMR (500 MHz, DMSO-d₆) δ 11.1 (s, 1H), 8.62 (d, J=2.9 Hz, 1H), 8.06 (d, J=1.8 Hz, 1H), 7.85-7.80 (m, 2H), 7.68-7.63 (m, 2H), 7.42-7.41 (dd, J=8.5, 1.9 Hz, 1H), 6.17-6.15 (dd, J=7.6, 2.7 Hz, 1H), 6.01 (d, J=2.8 Hz, 1H), 5.22 (s, 2H), 4.72 (d, J=16.1 Hz, 1H), 4.50-4.46 (d, J=16.1, 7.5 Hz, 1H), 3.82-3.75 (m, 1H), 3.53-3.43 (m, 1H), 3.19-3.16 (m, 2H), 2.97 (d, J=3.8 Hz, 3H); ESI MS m/z 422 [M+H]⁺; HPLC (Method B)>99% (AUC), t_{R}=10.1 min.

### Example 59

### Preparation of 1-(2-(2-Fluoroethyl)-1,2,3,4-tetrahydrobenzothieno[2,3-c]pyridin-7-yl)-4-((5-fluoropyridin-2-yl)methoxy)pyridin-2(1H)-one hydrochloride

### a) 7-Bromo-2-(2-fluoroethyl)-1,2,3,4-tetrahydrobenzothieno[2,3-c]pyridine

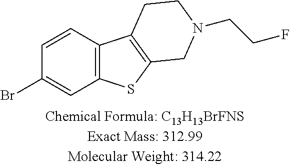 [SMILES: FCCN1CCc2c(sc3cc(Br)ccc23)C1]

tert-Butyl 7-bromo-3,4-dihydrobenzothieno[2,3-c]pyridine-2(1H)-carboxylate (350 mg, 0.951 mmol) was suspended in MeOH (3 mL) and 2 N HCl in Et₂O (10 mL) added. After 16 h, the mixture was concentrated, and the residue was dissolved in acetonitrile (10 mL). K₂CO₃ (656 mg, 4.75 mmol) and 2-bromofluoroethane (350 mg, 2.76 mmol) were added, and the mixture was heated at 80° C. for 72 h. CH₂Cl₂ (30 mL) and saturated NaHCO₃ solution (30 mL) were added, and the mixture was stirred for 1 h. The organic layer was removed, dried over sodium sulfate and concentrated to provide the crude title compound (320 mg, 100%) as an orange solid: ESI MS m/z 314 [M+H]⁺.

### b) 1-(2-(2-Fluoroethyl)-1,2,3,4-tetrahydrobenzothieno[2,3-c]pyridin-7-yl)-4-((5-fluoropyridin-2-yl)methoxy)pyridin-2(1H)-one hydrochloride

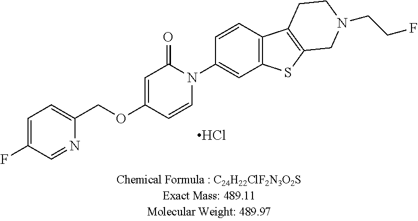 [SMILES: O=c1cc(OCc2ccc(F)cn2)ccn1-c1ccc2c3c(sc2c1)CN(CCF)CC3 ; Cl]

7-Bromo-2-(2-fluoroethyl)-1,2,3,4-tetrahydrobenzothieno[2,3-c]pyridine (320 mg, 0.951 mmol) and 4-((5-fluoropyridin-2-yl)methoxy)pyridin-2(1H)-one (220 mg, 0.951 mmol) were reacted according to Example 12 (step c) to provide the free-base (187 mg, 43%) as a white foam: ESI MS m/z 454 [M+H]⁺. The foam was converted to the hydrochloride according to Example 12 (step d) to provide the title compound (202 mg, 100%) as a white solid: ¹H NMR (500 MHz, DMSO-d₆) δ 11.36 (s, 1H), 8.62 (d, J=2.8 Hz, 1H), 8.07 (d, J=1.7 Hz, 1H), 7.84-7.80 (m, 2H), 7.67-7.63 (m, 2H), 7.43-7.41 (dd, J=8.4, 1.8 Hz, 1H), 6.18-6.16 (dd, J=7.6, 2.6 Hz, 1H), 6.01 (d, J=2.7 Hz, 1H), 5.27 (s, 2H), 5.03-5.02 (m, 1H), 4.94-4.92 (m, 1H), 4.85-4.74 (m, 1H), 4.65-4.57 (m, 1H), 3.94-3.63 (m, 4H), 3.23-3.18 (m, 2H); ESI MS m/z 454 [M+H]⁺; HPLC (Method B)>99% (AUC), t_{R}=10.6 min.

### Example 60

### Preparation of 4-(2-(5-Fluoropyridin-2-yl)ethyl)-1-(1,2,3,4-tetrahydrobenzothieno[2,3-c]pyridin-7-yl)piperazin-2-one hydrochloride

### a) tert-Butyl 7-(4-(2-(5-fluoropyridin-2-yl)ethyl)-2-oxopiperazin-1-yl)-3,4-dihydrobenzothieno[2,3-c]pyridine-2(1H)-carboxylate

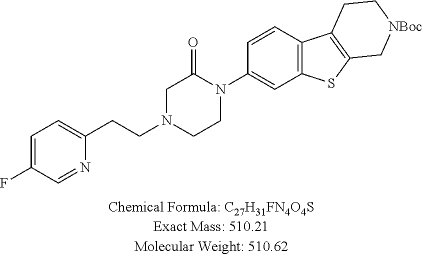 [SMILES: CC(C)(C)OC(=O)N1CCc2c(sc3cc(N4CCN(CCc5ccc(F)cn5)CC4=O)ccc23)C1]

tert-Butyl 7-bromo-3,4-dihydrobenzothieno[2,3-c]pyridine-2(1H)-carboxylate (134 mg, 0.364 mmol) and 4-(2-(5-fluoropyridin-2-yl)ethyl)piperazin-2-one (85 mg, 0.36 mmol) were reacted according to Example 12 (step c) to provide the title compound (126 mg, 68%) as a white foam: ESI MS m/z 511 [M+H]⁺.

### b) 4-(2-(5-Fluoropyridin-2-yl)ethyl)-1-(1,2,3,4-tetrahydrobenzothieno[2,3-c]pyridin-7-yl)piperazin-2-one hydrochloride

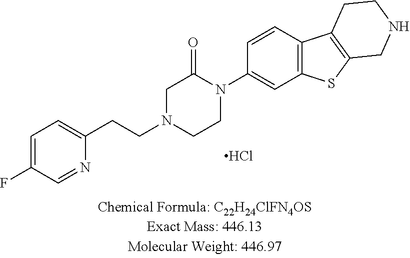 [SMILES: O=C1CN(CCc2ccc(F)cn2)CCN1c1ccc2c3c(sc2c1)CNCC3 ; Cl]

tert-Butyl 7-(4-(2-(5-fluoropyridin-2-yl)ethyl)-2-oxopiperazin-1-yl)-3,4-dihydrobenzothieno[2,3-c]pyridine-2(1H)-carboxylate (126 mg, 0.246 mmol) was deprotected and converted to the hydrochloride according to Example 12 (step d) to provide the title compound (65 mg, 59%) as a white solid: ¹H NMR (500 MHz, CD₃OD) δ 8.40 (d, J=2.7 Hz, 1H), 7.88 (d, J=1.4 Hz, 1H), 7.80 (dd, J=8.4, Hz, 1H), 7.60-7.56 (td, J=8.4, 2.9 Hz, 1H), 7.46-7.43 (dd, J=8.4, 4.4 Hz, 1H), 7.40-7.43 (dd, J=8.5, 1.7 Hz, 1H), 4.55 (s, 2H), 3.81-3.78 (m, 2H), 3.65 (t, J=6.1 Hz, 2H), 3.61-3.53 (m, 2H), 3.19-3.09 (m, 8H); ESI MS m/z 411 [M+H]⁺; HPLC (Method B)>99% (AUC), t_{R}=6.3 min.

### Example 61

### Preparation of 5-((5-Fluoropyridin-2-yl)methoxy)-2-(1,2,3,4-tetrahydrobenzothieno[2,3-c]pyridin-7-yl)pyridazin-3(2H)-one

### a) 5-((5-Fluoropyridin-2-yl)methoxy)pyridazin-3(2H)-one

CAS Registry Number 1008518-12-4

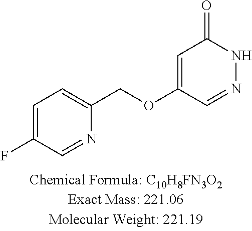 [SMILES: O=c1cc(OCc2ccc(F)cn2)cn[nH]1]

This compound was prepared in accordance with the procedure described in PCT Publication No. WO WO 2011/003007 to Guzzo et al., which is hereby incorporated by reference in its entirety.

### b) tert-Butyl 7-(4-((5-fluoropyridin-2-yl)methoxy)-6-oxopyridazin-1(6H)-yl)-3,4-dihydrobenzothieno[2,3-c]pyridine-2(1H)-carboxylate

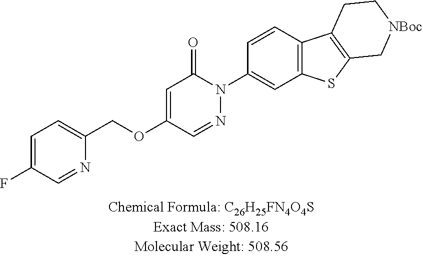 [SMILES: CC(C)(C)OC(=O)N1CCc2c(sc3cc(-n4ncc(OCc5ccc(F)cn5)cc4=O)ccc23)C1]

tert-Butyl 7-bromo-3,4-dihydrobenzothieno[2,3-c]pyridine-2(1H)-carboxylate (128 mg, 0.348 mmol) and 5-((5-fluoropyridin-2-yl)methoxy)pyridazin-3(2H)-one (77 mg, 0.35 mmol) were reacted according to Example 12 (step c) to provide the title compound (123 mg, 69%) as a white solid: ESI MS m/z 509 [M+H]⁺.

### b) 5-((5-Fluoropyridin-2-yl)methoxy)-2-(1,2,3,4-tetrahydrobenzothieno[2,3-c]pyridin-7-yl)pyridazin-3(2H)-one

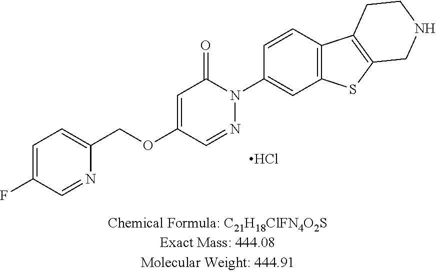 [SMILES: O=c1cc(OCc2ccc(F)cn2)cnn1-c1ccc2c3c(sc2c1)CNCC3 ; Cl]

tert-Butyl 7-(4-((5-fluoropyridin-2-yl)methoxy)-6-oxopyridazin-1(6H)-yl)-3,4-dihydrobenzothieno[2,3-c]pyridine-2(1H)-carboxylate (123 mg, 0.242 mmol) was deprotected and converted to the hydrochloride according to Example 12 (step d) to provide the title compound (63 mg, 59%) as a white solid: ¹H NMR (500 MHz, DMSO-d₆) δ 9.45 (s, 2H), 8.64 (d, J=2.8 Hz, 1H), 8.18 (d, J=1.8 Hz, 1H), 8.04 (d, J=2.8 Hz, 1H), 7.87-7.81 (m, 2H), 7.72-7.69 (dd, J=8.6, 4.5, 1H), 7.57-7.55 (dd, J=8.5, 1.8 Hz, 1H), 6.58 (d, J=2.8 Hz, 1H), 5.30 (s, 2H), 4.50 (s, 2H), 3.53 (t, J=5.8 Hz, 2H), 3.09 (t, J=5.8 Hz, 2H); ESI MS m/z 409 [M+H]⁺; HPLC (Method B)>99% (AUC), t_{R}=10.3 min.

### Example 62

### Preparation of 4-((6-Methylpyridin-3-yl)methoxy)-1-(1,2,3,4-tetrahydrobenzothieno[2,3-c]pyridin-7-yl)pyridin-2(1H)-one hydrochloride

### a) 4-((6-Methylpyridin-3-yl)methoxy)pyridin-2(1H)-one

CAS Registry Number 1260240-53-6

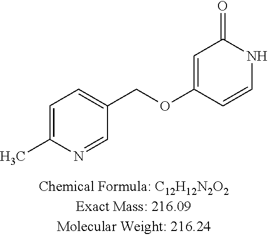 [SMILES: Cc1ccc(COc2cc[nH]c(=O)c2)cn1]

This compound was prepared in accordance with the procedure described in PCT Publication No. WO 2011/003012 to Guzzo et al., which is hereby incorporated by reference in its entirety.

### b) tert-Butyl 7-(4-((6-methylpyridin-3-yl)methoxy)-2-oxopyridin-1(2H)-yl)-3,4-dihydrobenzothieno[2,3-c]pyridine-2(1H)-carboxylate

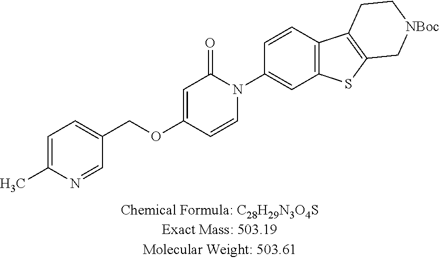 [SMILES: Cc1ccc(COc2ccn(-c3ccc4c5c(sc4c3)CN(C(=O)OC(C)(C)C)CC5)c(=O)c2)cn1]

tert-Butyl 7-bromo-3,4-dihydrobenzothieno[2,3-c]pyridine-2(1H)-carboxylate (90 mg, 0.24 mmol) and 4-((6-methylpyridin-3-yl)methoxy)pyridin-2(1H)-one (53 mg, 0.24 mmol) were reacted according to Example 12 (step c) to provide the title compound (102 mg, 54%) as a white solid: ESI MS m/z 504 [M+H]⁺.

### b) 4-((6-Methylpyridin-3-yl)methoxy)-1-(1,2,3,4-tetrahydrobenzothieno[2,3-c]pyridin-7-yl)pyridin-2(1H)-one hydrochloride

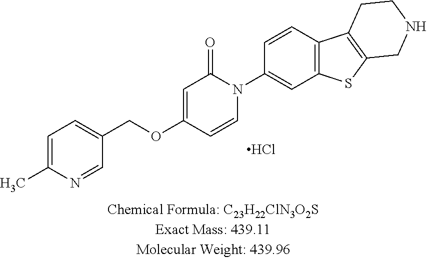 [SMILES: Cc1ccc(COc2ccn(-c3ccc4c5c(sc4c3)CNCC5)c(=O)c2)cn1 ; Cl]

tert-Butyl 7-(4-((6-methylpyridin-3-yl)methoxy)-2-oxopyridin-1(2H)-yl)-3,4-dihydrobenzothieno[2,3-c]pyridine-2(1H)-carboxylate (102 mg, 0.202 mmol) was deprotected and converted to the hydrochloride according to Example 12 (step d) to provide the title compound (63 mg, 70%) as a white solid: ¹H NMR (500 MHz, DMSO-d₆) δ 9.80 (s, 2H), 8.70 (s, 1H), 8.10 (d, J=7.6 Hz, 1H), 8.05 (d, J=1.8 Hz, 1H), 7.83 (d, J=8.4 Hz, 1H), 7.65 (d, J=7.6, 1H), 7.60 (d, J=8.0 Hz, 1H), 7.41-7.39 (dd, J=8.4, 1.8 Hz, 1H), 6.15-6.13 (dd, J=7.6, 2.7 Hz, 1H), 6.08 (d, J=2.6 Hz, 1H), 5.24 (s, 2H), 4.48 (s, 2H), 3.53-3.50 (m, 2H), 3.10-3.06 (m, 2H), 2.64 (s, 3H); ESI MS m/z 404 [M+H]⁺; HPLC (Method B)>99% (AUC), t_{R}=6.6 min.

### Example 63

### Preparation of 4-(4-Chlorophenethyl)-1-(1,2,3,4-tetrahydrobenzothieno[2,3-c]pyridin-7-yl)piperazin-2-one hydrochloride

### a) tert-Butyl 7-(4-(4-chlorophenethyl)-2-oxopiperazin-1-yl)-3,4-dihydrobenzothieno[2,3-c]pyridine-2(1H)-carboxylate

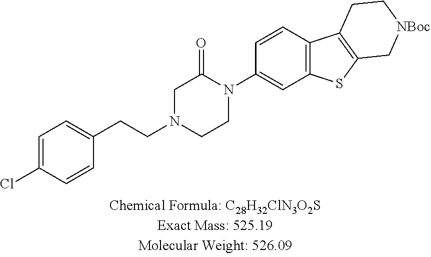 [SMILES: CC(C)(C)OC(=O)N1CCc2c(sc3cc(N4CCN(CCc5ccc(Cl)cc5)CC4=O)ccc23)C1]

tert-Butyl 7-bromo-3,4-dihydrobenzothieno[2,3-c]pyridine-2(1H)-carboxylate (138 mg, 0.375 mmol) and 4-(4-chlorophenethyl)piperazin-2-one (90 mg, 0.37 mmol) were reacted according to Example 12 (step c) to provide the title compound (40 mg, 20%) as a white foam: ESI MS m/z 526 [M+H]⁺.

### b) 4-(4-Chlorophenethyl)-1-(1,2,3,4-tetrahydrobenzothieno[2,3-c]pyridin-7-yl)piperazin-2-one hydrochloride

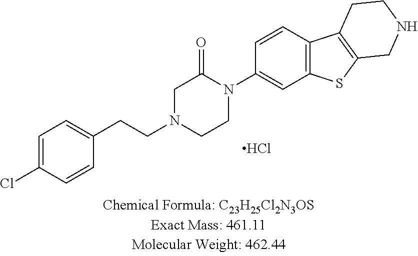 [SMILES: O=C1CN(CCc2ccc(Cl)cc2)CCN1c1ccc2c3c(sc2c1)CNCC3 ; Cl]

tert-Butyl 7-(4-(4-chlorophenethyl)-2-oxopiperazin-1-yl)-3,4-dihydrobenzothieno[2,3-c]pyridine-2(1H)-carboxylate (40 mg, 0.076 mmol) was deprotected and converted to the hydrochloride according to Example 12 (step d) to provide the title compound (29 mg, 82%) as a white solid: ¹H NMR (500 MHz, CD₃OD) δ 7.92 (d, J=1.6 Hz, 1H), 7.81 (d, J=8.6 Hz, 1H), 7.44-7.42 (dd, J=8.5, 1.6 Hz, 1H), 7.37-7.31 (m, 4H), 4.56 (s, 2H), 4.20-3.37 (m, 9H), 3.21-3.01 (m, 5H); ESI MS m/z 426 [M+H]⁺; HPLC (Method B)>99% (AUC), t_{R}=8.7 min.

### Example 64

### Preparation of 1-(1,2,3,4-Tetrahydrobenzothieno[2,3-c]pyridin-7-yl)-4-((6-(trifluoromethyl)pyridin-3-yl)methoxy)pyridin-2(1H)-one hydrochloride

### a) 4-((6-(Trifluoromethyl)pyridin-3-yl)methoxy)pyridin-2(1H)-one

CAS Registry Number 1173155-96-8

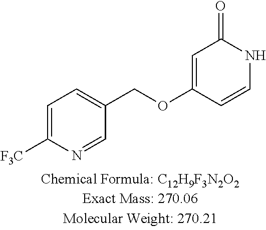 [SMILES: O=c1cc(OCc2ccc(C(F)(F)F)nc2)cc[nH]1]

This compound was prepared in accordance with the procedure described in PCT Publication No. WO 2009/089482 to Guzzo et al., which is hereby incorporated by reference in its entirety.

### b) tert-Butyl 7-(2-oxo-4-((6-(trifluoromethyl)pyridin-1(2H)-yl)-3,4-dihydrobenzothieno[2,3-c]pyridine-2(1H)-carboxylate

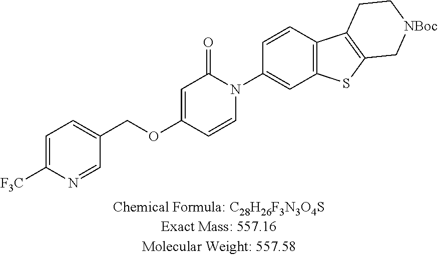 [SMILES: CC(C)(C)OC(=O)N1CCc2c(sc3cc(-n4ccc(OCc5ccc(C(F)(F)F)nc5)cc4=O)ccc23)C1]

tert-Butyl 7-bromo-3,4-dihydrobenzothieno[2,3-c]pyridine-2(1H)-carboxylate (125 mg, 0.340 mmol) and 4-((6-(trifluoromethyl)pyridin-3-yl)methoxy)pyridin-2(1H)-one (92 mg, 0.34 mmol) were reacted according to Example 12 (step c) to provide the title compound (140 mg, 74%) as a white solid: ESI MS m/z 558 [M+H]⁺.

### c) 1-(1,2,3,4-Tetrahydrobenzothieno[2,3-c]pyridin-7-yl)-4-((6-(trifluoromethyl)pyridin-3-yl)methoxy)pyridin-2(1H)-one hydrochloride

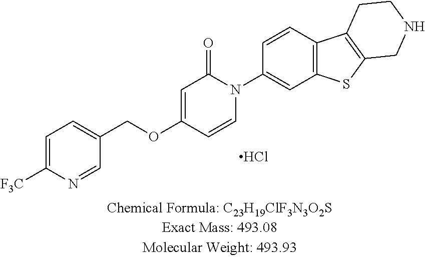 [SMILES: O=c1cc(OCc2ccc(C(F)(F)F)nc2)ccn1-c1ccc2c3c(sc2c1)CNCC3 ; Cl]

tert-Butyl 7-(2-oxo-4-((6-(trifluoromethyl)pyridin-3-yl)methoxy)pyridin-1(2H)-yl)-3,4-dihydrobenzothieno[2,3-c]pyridine-2(1H)-carboxylate (140 mg, 0.283 mmol) was deprotected and converted to the hydrochloride according to Example 12 (step d) to provide the title compound (93 mg, 66%) as a white solid: ¹H NMR (500 MHz, DMSO-d₆) δ 9.62 (s, 2H), 8.89 (s, 1H), 8.19 (d, J=7.9 Hz, 1H), 8.06 (d, J=1.8 Hz, 1H), 7.99 (d, J=8.0 Hz, 1H), 7.83 (d, J=8.4, 1H), 7.66 (d, J=7.6 Hz, 1H), 7.41-7.40 (dd, J=8.4, 1.8 Hz, 1H), 6.19-6.17 (dd, J=7.6, 2.7 Hz, 1H), 6.05 (d, J=2.7 Hz, 1H), 5.35 (s, 2H), 4.49 (s, 2H), 3.53-3.50 (m, 2H), 3.09-3.07 (m, 2H); ESI MS m/z 458 [M+H]⁺; HPLC (Method B)>99% (AUC), t_{R}=14.5 min.

### Example 65

### Preparation of 4-Benzyloxy-1-(1,2,3,4-tetrahydrobenzo[4,5]thieno[2,3-c]pyridin-6-yl)pyridin-2(1H)-one hydrochloride

### a) tert-Butyl 3-(4-bromophenylthio)-4-hydroxypiperidine-1-carboxylate and tert-butyl 4-(4-bromophenyl)thio-3-hydroxypiperidine-1-carboxylate

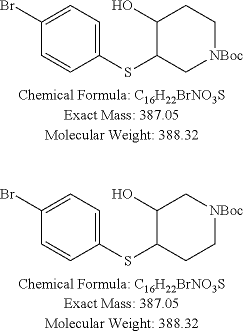 [SMILES: CC(C)(C)OC(=O)N1CCC(O)C(Sc2ccc(Br)cc2)C1 ; CC(C)(C)OC(=O)N1CCC(Sc2ccc(Br)cc2)C(O)C1]

4-Bromobenzenethiol (12.0 g, 63.1 mmol) and sodium hydroxide (2.5 g, 63 mmol) were dissolved in MeOH (40 mL) at ambient temperature and treated with tert-butyl 7-oxa-3-azabicyclo[4.1.0]heptane-3-carboxylate (12.6 g, 63.1 mmol). The reaction mixture was heated at reflux for 1 h and concentrated in vacuo. The residue was purified by flash column chromatography (silica gel, hexanes/EtOAc, 90:10 to 70:30) to afford tert-butyl 3-(4-bromophenylthio)-4-hydroxypiperidine-1-carboxylate (4.3 g, 17%, minor regioisomer) as a colorless oil: ¹H NMR (300 MHz, CDCl₃) δ 7.44-7.41 (m, 2H), 7.34-7.31 (m, 2H), 4.22 (br s, 1H), 4.02 (d, J=13.2 Hz, 1H), 3.51 (br s, 1H), 3.02 (br s, 1H), 2.91-2.77 (m, 3H), 2.09-2.03 (m, 1H), 1.42 (s, 9H); and tert-butyl 4-(4-bromophenyl)thio-3-hydroxypiperidine-1-carboxylate (18.3 g, 74%, major regioisomer) as a colorless oil: ¹H NMR (300 MHz, CDCl₃) δ 7.46-7.43 (m, 2H), 7.35-7.31 (m, 2H), 4.27-4.21 (m, 1H), 3.93 (br s, 1H), 3.41 (br s, 1H), 2.90-2.72 (m, 4H), 2.05-2.00 (m, 1H), 1.43 (s, 9H).

### b) tert-Butyl 3-(4-bromophenyl)thio-4-oxopiperidine-1-carboxylate

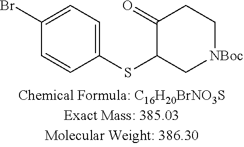 [SMILES: CC(C)(C)OC(=O)N1CCC(=O)C(Sc2ccc(Br)cc2)C1]

tert-Butyl 3-(4-bromophenylthio)-4-hydroxypiperidine-1-carboxylate (1.6 g, 4.1 mmol) and Dess-Martin periodinane (2.4 g, 5.7 mmol) were reacted according to Example 124 (step c) to provide the title compound (1.0 g, 63%) as a colorless oil: ¹H NMR (CDCl₃, 300 MHz) δ 7.43-7.39 (m, 2H), 7.30-7.27 (m, 2H), 3.96-3.90 (m, 1H), 3.81-3.69 (m, 4H), 2.90-2.81 (m, 1H), 2.44-2.31 (m, 1H), 1.48 (s, 9H).

### c) tert-Butyl 6-bromo-3,4-dihydrobenzo[4,5]thieno[2,3-c]pyridine-2(1H)-carboxylate

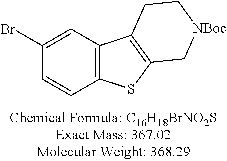 [SMILES: CC(C)(C)OC(=O)N1CCc2c(sc3ccc(Br)cc23)C1]

tert-Butyl 3-(4-bromophenyl)thio-4-oxopiperidine-1-carboxylate (1.0 g, 2.6 mmol) and polyphosphoric acid (3.8 g) were reacted according to Example 16 (step b) to provide the title compound (300 mg, 32%) as a colorless oil: ¹H NMR (CDCl₃, 300 MHz) δ 7.69 (d, J=1.5 Hz, 1H), 7.61 (d, J=8.4 Hz, 1H), 7.38-7.35 (m, 1H), 4.69 (s, 2H), 3.78 (t, J=5.7 Hz, 2H), 2.78 (t, J=5.7 Hz, 2H), 1.50 (s, 9H).

### d) tert-Butyl 6-(4-benzyloxy-2-oxopyridin-1(2H)-yl)-3,4-dihydrobenzo[4,5]thieno[2,3-c]pyridine-2(1H)-carboxylate

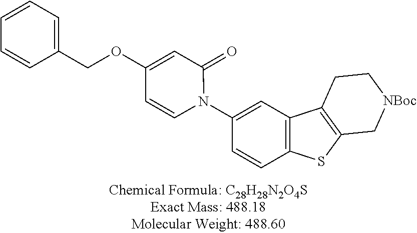 [SMILES: CC(C)(C)OC(=O)N1CCc2c(sc3ccc(-n4ccc(OCc5ccccc5)cc4=O)cc23)C1]

A mixture of tert-butyl 6-bromo-3,4-dihydrobenzo[4,5]thieno[2,3-c]pyridine-2(1H)-carboxylate (300 mg, 0.84 mmol), 4-benzyloxypyridin-2(1H)-one (220 mg, 0.92 mmol), CuI (130 mg, 0.97 mmol), 8-hydroxyquinoline (37 mg, 0.25 mmol) and K₂CO₃ (80 mg, 0.42 mmol) in DMSO (10.0 mL) was degassed with a nitrogen stream for 10 min. The suspension was heated at 120° C. for 12 h under a N₂ atmosphere. The reaction mixture cooled to ambient temperature and diluted with 10% NH₄OH in H₂O (25 mL) and CH₂Cl₂ (40 mL). The organic layer was separated, dried over Na₂SO₄ and concentrated in vacuo. Purification by flash column chromatography (silica gel, hexanes/EtOAc, 80:20 to 50:50) afforded the title compound (86 mg, 22%) as a colorless oil: ¹H NMR (300 MHz, CDCl₃) δ 7.85 (d, J=8.4 Hz, 1H), 7.55 (d, J=1.8 Hz, 1H), 7.37-7.26 (m, 7H), 6.08-6.05 (m, 2H), 5.05 (d, J=5.7 Hz, 2H), 4.71 (s, 2H), 3.78 (t, J=5.6 Hz, 2H), 2.82 (t, J=5.7 Hz, 2H), 1.50 (s, 9H).

### e) 4-Benzyloxy-1-(1,2,3,4-tetrahydrobenzo[4,5]thieno[2,3-c]pyridin-6-yl)pyridin-2(1H)-one hydrochloride

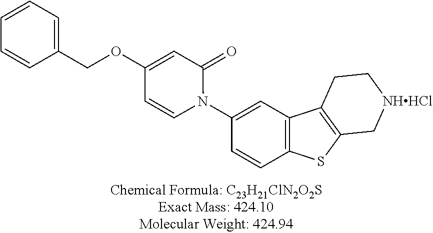

A solution of tert-Butyl 6-(4-benzyloxy-2-oxopyridin-1(2H)-yl)-3,4-dihydrobenzo[4,5]thieno[2,3-c]pyridine-2(1H)-carboxylate (86 mg, 0.17 mmol) in THF (10 mL) was treated with concentrated aqueous HCl (3.0 mL). After stirring for 2 h, the mixture was concentrated and partitioned between 10% aqueous NaOH solution (5 mL) and CH₂Cl₂ (30 mL). The organic layer was removed, dried over Na₂SO₄ and concentrated in vacuo. Purification by flash column chromatography (silica gel, EtOAc/MeOH, 100:0 to 80:20) afforded the free base (65 mg, 88%) as a white solid. The solid was suspended in MeOH (5 mL) and treated with 1.25 M HCl in MeOH. After 10 min, the mixture was concentrated in vacuo and lyophilized from water to provide the title compound (70 mg, 99%) as a white solid: mp 170° C.; ¹H NMR (300 MHz, DMSO-d₆) δ 9.51 (s, 2H), 8.09 (d, J=8.7 Hz, 1H), 7.75 (d, J=1.8 Hz, 1H), 7.64 (d, J=7.8 Hz, 1H), 7.49-7.36 (m, 6H), 6.16-6.13 (m, 1H), 6.00 (d, J=2.7 Hz, 1H), 5.16 (s, 2H), 4.50 (s, 2H), 3.51 (s, 2H), 3.04 (br s, 2H); ESI MS m/z 389 [M+H]⁺; HPLC (Method C) 98.9% (AUC), t_{R}=20.8 min.

### Example 66

### Preparation of 4-((5-Fluoropyridin-2-yl)methoxy)-1-(1,2,3,4-tetrahydrobenzo[4,5]thieno[2,3-c]pyridin-6-yl)pyridin-2(1H)-one hydrochloride

### a) tert-Butyl 6-(4-((5-fluoropyridin-2-yl)methoxy)-2-oxopyridin-1(2H)-yl)-3,4-dihydrobenzo[4,5]thieno[2,3-c]pyridine-2(1H)-carboxylate

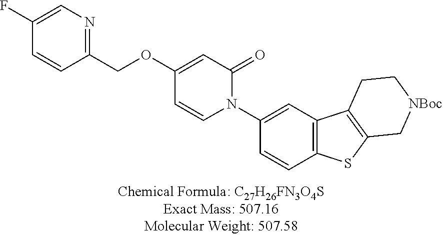 [SMILES: CC(C)(C)OC(=O)N1CCc2c(sc3ccc(-n4ccc(OCc5ccc(F)cn5)cc4=O)cc23)C1]

tert-Butyl 6-bromo-3,4-dihydrobenzo[4,5]thieno[2,3-c]pyridine-2(1H)-carboxylate (380 mg, 1.1 mmol) and 4-((5-fluoropyridin-2-yl)methoxy)pyridin-2(1H)-one (296 mg, 1.34 mmol) were coupled using the procedure of Example 65 (step d) to provide the title compound (160 mg, 30%) as a white solid: ¹H NMR (300 MHz, CDCl₃) δ 8.49 (s, 1H), 7.85 (d, J=8.1 Hz, 1H), 7.56 (s, 1H), 7.49-7.46 (m, 2H), 7.32-7.25 (m, 2H), 6.12-6.03 (m, 2H), 5.17 (s, 2H), 4.71 (s, 2H), 3.78 (t, J=5.1 Hz, 2H), 2.82 (s, 2H), 1.50 (s, 9H).

### b) 4-((5-Fluoropyridin-2-yl)methoxy)-1-(1,2,3,4-tetrahydrobenzo[4,5]thieno[2,3-c]pyridin-6-yl)pyridin-2(1H)-one hydrochloride

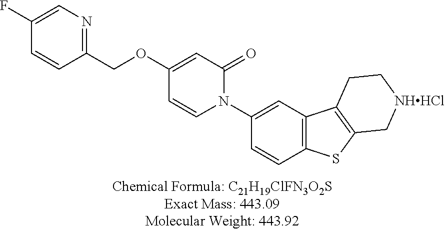

A solution of tert-butyl 6-(4-(5-fluoropyridin-2-ylmethylamino)-2-oxopyridin-1(2H)-yl)-3,4-dihydrobenzo[4,5]thieno[2,3-c]pyridine-2(1H)-carboxylate (160 mg, 0.30 mmol) in THF (10 mL) was treated with concentrated aqueous HCl solution (2.0 mL). After 2 h, the resulting precipitate was filtered, washed with THF and lyophilized from water to provide the title compound (120 mg, 85%) as a white solid: mp 278-280° C.; ¹H NMR (400 MHz, DMSO-d₆) δ 9.69 (s, 2H), 8.62 (d, J=2.8 Hz, 1H), 8.08 (d, J=8.4 Hz, 1H), 7.86-7.81 (m, 1H), 7.75 (d, J=2.0 Hz, 1H), 7.67-7.64 (m, 2H), 7.38-7.36 (m, 1H), 6.19-6.16 (m, 1H), 6.00 (d, J=2.8 Hz, 1H), 5.23 (s, 2H), 4.46 (s, 2H), 3.50-3.48 (br s, 2H), 3.05 (s, 2H); APCI MS m/z 408 [M+H]⁺; HPLC (Method C)>99% (AUC), t_{R}=19.5 min.

### Example 67

### Preparation of 4-Benzyloxy-1-(1,2,3,4-tetrahydrobenzo[4,5]thieno[3,2-c]pyridin-8-yl)pyridin-2(1H)-one hydrochloride

### a) tert-Butyl 4-(4-bromophenyl)thio-3-oxopiperidine-1-carboxylate

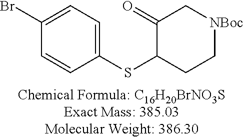 [SMILES: CC(C)(C)OC(=O)N1CCC(Sc2ccc(Br)cc2)C(=O)C1]

tert-Butyl 4-(4-bromophenyl)thio-3-hydroxypiperidine-1-carboxylate (19.4 g, 50.0 mmol) and Dess-Martin periodinane (27 g, 65 mmol) were reacted according to Example 124 (step c) to provide the title compound (16.8 g, 88%) as a colorless oil: ¹H NMR (300 MHz, CDCl₃) δ 7.45-7.42 (m, 2H), 7.31-7.28 (m, 2H), 4.31-4.05 (m, 2H), 3.82-3.78 (m, 1H), 3.69-3.63 (m, 2H), 2.41-2.39 (m, 1H), 2.32-2.08 (m, 1H), 1.48 (s, 9H).

### b) tert-Butyl 8-bromo-3,4-dihydrobenzo[4,5]thieno[3,2-c]pyridine-2(1H)-carboxylate

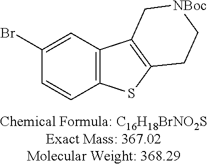 [SMILES: CC(C)(C)OC(=O)N1CCc2sc3ccc(Br)cc3c2C1]

tert-Butyl 4-(4-bromophenyl)thio-3-oxopiperidine-1-carboxylate (4.5 g, 12 mmol) was reacted with polyphosphoric acid (12 g) according to Example 16 (step b) to provide the title compound (350 mg, 8%) as a colorless oil: ¹H NMR (300 MHz, CDCl₃) δ 7.67-7.61 (m, 1H), 7.43-7.37 (m, 2H), 4.61 (s, 2H), 3.81 (t, J=5.4 Hz, 2H), 2.93 (t, J=5.1 Hz, 2H), 1.52 (s, 9H).

### c) tert-Butyl 8-(4-benzyloxy-2-oxopyridin-1(2H)-yl)-3,4-dihydrobenzo[4,5]thieno[3,2-c]pyridine-2(1H)-carboxylate

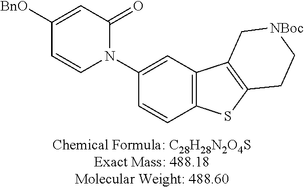 [SMILES: CC(C)(C)OC(=O)N1CCc2sc3ccc(-n4ccc(OCc5ccccc5)cc4=O)cc3c2C1]

tert-Butyl 8-bromo-3,4-dihydrobenzo[4,5]thieno[3,2-c]pyridine-2(1H)-carboxylate (140 mg, 0.38 mmol) and 4-benzyloxypyridin-2(1H)-one (80 mg, 0.4 mmol) were coupled using the procedure of Example 65 (step d) to provide the title compound (151 mg, 76%) as a colorless oil: ¹H NMR (300 MHz, CDCl₃) δ 7.85 (d, J=8.7 Hz, 1H), 7.51 (s, 1H), 7.43-7.37 (m, 5H), 7.30-7.26 (m, 2H), 6.09-6.04 (m, 2H), 5.05 (d, J=7.2 Hz, 2H), 4.64 (s, 2H), 3.81 (t, J=5.4 Hz, 2H), 2.95 (t, J=5.1 Hz, 2H), 1.50 (s, 9H).

### d) 4-Benzyloxy-1-(1,2,3,4-tetrahydrobenzo[4,5]thieno[3,2-c]pyridin-8-yl)pyridin-2(1H)-one hydrochloride

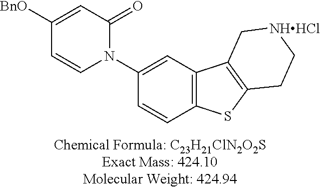

tert-Butyl 8-(4-benzyloxy-2-oxopyridin-1(2H)-yl)-3,4-dihydrobenzo[4,5]thieno[3,2-c]pyridine-2(1H)-carboxylate (150 mg, 0.27 mmol) was treated with concentrated aqueous HCl according to Example 65 (step e) to provide the title compound (102 mg, 70%) as a white solid: mp 245-247° C.; ¹H NMR (300 MHz, DMSO-d₆) δ 9.73 (s, 2H), 8.06 (d, J=8.4 Hz, 1H), 7.77 (d, J=2.1 Hz, 1H), 7.63 (d, J=7.5 Hz, 1H), 7.49-7.35 (m, 6H), 6.16-6.13 (m, 1H), 6.00 (d, J=2.7 Hz, 1H), 5.17 (s, 2H), 4.37 (s, 2H), 3.50 (br s, 2H), 3.19 (t, J=4.8 Hz, 2H); ESI MS m/z 389 [M+H]⁺; HPLC (Method C) 98.6% (AUC), t_{R}=20.8 min.

### Example 68

### Preparation of 4-(5-Fluoropyridin-2-yl)methoxy-1-(1,2,3,4-tetrahydrobenzo[4,5]thieno[3,2-c]pyridin-8-yl)pyridin-2(1H)-one hydrochloride

### a) tert-Butyl 8-(4-((5-fluoropyridin-2-yl)methoxy)-2-oxopyridin-1(2H)-yl)-3,4-dihydrobenzo[4,5]thieno[3,2-c]pyridine-2(1H)-carboxylate

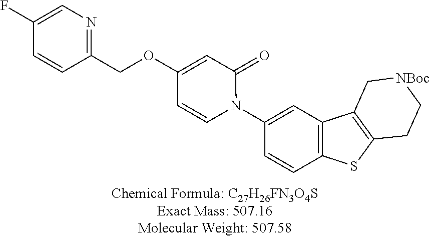 [SMILES: CC(C)(C)OC(=O)N1CCc2sc3ccc(-n4ccc(OCc5ccc(F)cn5)cc4=O)cc3c2C1]

tert-Butyl 8-bromo-3,4-dihydrobenzo[4,5]thieno[3,2-c]pyridine-2(1H)-carboxylate (140 mg, 0.38 mmol) and 4-((5-fluoropyridin-2-yl)methoxy)pyridin-2(1H)-one (88 mg, 0.40 mmol) were coupled according to Example 65 (step d) to provide the title compound (166 mg, 77%) as a white solid: ¹H NMR (300 MHz, CDCl₃) δ 8.48 (s, 1H), 7.85 (d, J=8.4 Hz, 1H), 7.51-7.37 (m, 3H), 7.31-7.21 (m, 2H), 6.14-6.03 (m, 2H), 5.16 (d, J=6.9 Hz, 2H), 4.64 (s, 2H), 3.81 (t, J=5.1 Hz, 2H), 2.95 (s, 2H), 1.50 (s, 9H).

### b) 4-(5-Fluoropyridin-2-yl)methoxy-1-(1,2,3,4-tetrahydrobenzo[4,5]thieno[3,2-c]pyridin-8-yl)pyridin-2(1H)-one hydrochloride

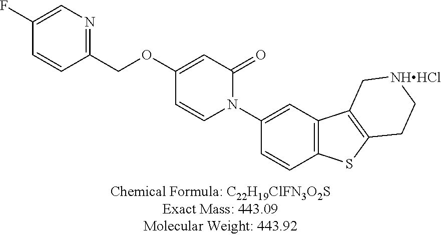

tert-Butyl 8-(4-((5-fluoropyridin-2-yl)methoxy)-2-oxopyridin-1(2H)-yl)-3,4-dihydrobenzo[4,5]thieno[3,2-c]pyridine-2(1H)-carboxylate was treated with concentrated aqueous HCl according to Example 65 (step e) to provide the title compound (105 mg, 75%) as a white solid: mp 165-167° C.; ¹H NMR (300 MHz, DMSO-d₆) δ 9.88 (br s, 2H), 8.62 (d, J=2.7 Hz, 1H), 8.06 (d, J=8.4 Hz, 1H), 7.87-7.78 (m, 2H), 7.68-7.64 (m, 2H), 7.38-7.35 (m, 1H), 6.20-6.16 (m, 1H), 6.01 (d, J=2.7 Hz, 1H), 5.23 (s, 2H), 4.37 (s, 2H), 3.50 (br s, 2H), 3.20 (t, J=5.1 Hz, 2H); APCI MS m/z 408 [M+H]⁺; HPLC (Method C)>99% (AUC), t_{R}=19.2 min.

### Example 69

### Preparation of 4-(5-Fluoropyridin-2-yl)methoxy-1-(2,3,4,5-tetrahydro-1H-benzo[4,5]thieno[2,3-d]azepin-9-yl)pyridin-2(1H)-one hydrochloride

### a) tert-Butyl 4-(4-bromophenyl)thio-5-oxoazepane-1-carboxylate and tert-butyl 3-(4-bromophenyl)thio-4-oxoazepane-1-carboxylate

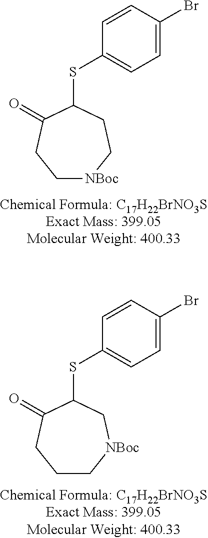 [SMILES: CC(C)(C)OC(=O)N1CCC(=O)C(Sc2ccc(Br)cc2)CC1 ; CC(C)(C)OC(=O)N1CCCC(=O)C(Sc2ccc(Br)cc2)C1]

tert-Butyl 4-oxoazepane-1-carboxylate (7.01 g, 32.9 mmol) coupled with 4-bromobenzenethiol (7.50 g, 39.5 mmol) according to Example 12 (step a) to provide a 3:2 inseparable mixture of tert-butyl 4-(4-bromophenyl)thio-5-oxoazepane-1-carboxylate and tert-butyl 3-(4-bromophenyl)thio-4-oxoazepane-1-carboxylate (2.4 g, 18% combined yield) as a brown oil: ¹H NMR (300 MHz, CDCl₃) δ 7.51-7.41 (m, 2H), 7.31-7.23 (m, 2H), 4.43-4.25 (m, 0.4H), 4.16-4.01 (m, 0.6H), 3.85-3.82 (m, 1H), 3.71-3.49 (m, 1H), 3.10-2.82 (m, 2.5H), 2.69-2.49 (m, 0.5H), 2.44-2.42 (m, 0.5H), 2.23-2.17 (m, 0.5H), 1.98-1.75 (m, 2H), 1.56 (s, 5H), 1.45 (s, 4H).

### b) 9-Bromo-2,3,4,5-tetrahydro-1H-benzo[4,5]thieno[2,3-d]azepine and 7-bromo-2,3,4,5-tetrahydro-1H-benzo[4,5]thieno[2,3-c]azepine

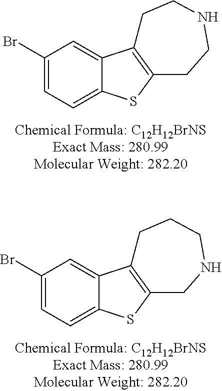 [SMILES: Brc1ccc2sc3c(c2c1)CCNCC3 ; Brc1ccc2sc3c(c2c1)CCCNC3]

A mixture tert-butyl 4-(4-bromophenyl)thio-5-oxoazepane-1-carboxylate and tert-butyl 3-(4-bromophenyl)thio-4-oxoazepane-1-carboxylate (2.4 g, 6.0 mmol) was reacted with polyphosphoric acid (11 g) according to Example 12 (step b) to provide 9-bromo-2,3,4,5-tetrahydro-1H-benzo[4,5]thieno[2,3-d]azepine (372 mg, 20%, major regio-isomer) as a colorless oil: ¹H NMR (300 MHz, CDCl₃) δ 7.72 (d, J=1.8 Hz, 1H), 7.58 (d, J=8.7 Hz, 1H), 7.36-7.33 (m, 1H), 3.10-3.06 (m, 4H), 3.04-3.01 (m, 2H), 2.97-2.93 (m, 2H); and 7-bromo-2,3,4,5-tetrahydro-1H-benzo[4,5]thieno[2,3-c]azepine (369 mg, 10%, minor regio-isomer) as a colorless oil: ¹H NMR (300 MHz, CDCl₃) δ 7.76 (d, J=1.8 Hz, 1H), 7.60 (d, J=8.4 Hz, 1H), 7.38-7.34 (m, 1H), 4.07 (s, 2H), 3.29-3.25 (m, 2H), 2.97-2.94 (m, 2H), 1.88-1.80 (m, 2H).

### c) tert-Butyl 9-bromo-4,5-dihydro-1H-benzo[4,5]thieno[2,3-d]azepine-3(2H)-carboxylate

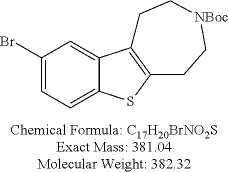 [SMILES: CC(C)(C)OC(=O)N1CCc2sc3ccc(Br)cc3c2CC1]

A mixture of 9-bromo-2,3,4,5-tetrahydro-1H-benzo[4,5]thieno[2,3-d]azepine (370 mg, 1.3 mmol), triethylamine (0.42 mL, 3.0 mmol) and di-tert-butyl dicarbonate (440 mg, 2.0 mmol) in MeOH (10 mL) was stirred at ambient for 2 h, then the mixture was concentrated in vacuo. The residue was purified by flash column chromatography (silica gel, hexanes/EtOAc, 99:1 to 95:5) to afford the title compound (452 mg, 91%) as a colorless oil: ¹H NMR (300 MHz, CDCl₃) δ 7.73 (d, J=1.8 Hz, 1H), 7.60 (d, J=2.4 Hz, 1H), 7.39-7.35 (m, 1H), 3.69 (br s, 4H), 3.08 (br s, 2H), 2.99 (br s, 2H), 1.50 (s, 9H).

### d) tert-Butyl 8-(4-benzyloxy-2-oxopyridin-1(2H)-yl)-3,4-dihydrobenzo[4,5]thieno[3,2-c]pyridine-2(1H)-carboxylate

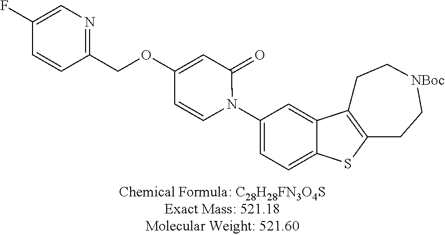 [SMILES: CC(C)(C)OC(=O)N1CCc2sc3ccc(-n4ccc(OCc5ccc(F)cn5)cc4=O)cc3c2CC1]

tert-Butyl 9-bromo-4,5-dihydro-1H-benzo[4,5]thieno[2,3-d]azepine-3(2H)-carboxylate (280 mg, 0.73 mmol) and 4-(5-fluoropyridin-2-yl)methoxypyridin-2(1H)-one (165 mg, 0.749 mmol) were coupled according to Example 65 (step d) to provide the title compound (101 mg, 40%) as a colorless oil: ¹H NMR (300 MHz, CDCl₃) δ 8.48 (d, J=2.1 Hz, 1H), 7.81 (d, J=8.4 Hz, 1H), 7.59-7.45 (m, 3H), 7.32-7.22 (m, 2H), 6.12-6.06 (m, 2H), 5.17 (s, 2H), 3.69 (br s, 4H), 3.09 (br s, 2H), 2.99 (br s, 2H), 1.50 (s, 9H).

### f) 4-(5-Fluoropyridin-2-yl)methoxy-1-(2,3,4,5-tetrahydro-1H-benzo[4,5]thieno[2,3-d]azepin-9-yl)pyridin-2(1H)-one hydrochloride

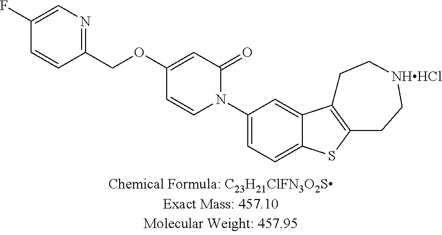

tert-Butyl 8-(4-benzyloxy-2-oxopyridin-1(2H)-yl)-3,4-dihydrobenzo[4,5]thieno[3,2-c]pyridine-2(1H)-carboxylate was treated with concentrated aqueous HCl according to Example 65 (step e) to provide the title compound (74 mg, 85%) as a white solid: mp 228-230° C.; ¹H NMR (300 MHz, DMSO-d₆) δ 9.48 (br s, 2H), 8.62 (d, J=3.0 Hz, 1H), 8.00 (d, J=5.7 Hz, 1H), 7.86-7.79 (m, 1H), 7.74 (d, J=1.8 Hz, 1H), 7.68-7.61 (m, 2H), 7.30 (dd, J=8.4, 2.0 Hz, 1H), 6.16 (dd, J=6.9, 2.7 Hz, 1H), 6.00 (d, J=2.7 Hz, 1H), 5.23 (s, 2H), 3.33 (br s, 6H), 3.28 (br s, 2H); APCI MS m/z 422 [M+H]⁺; HPLC (Method C)>99% (AUC), t_{R}=27.0 min.

### Example 70

### Preparation of 4-(2-(5-Fluoropyridin-2-yl)ethyl)-1-(1,2,3,4-tetrahydrobenzofuro[3,2-c]pyridin-7-yl)piperazin-2-one hydrochloride

### a) 2-(5-Fluoropyridin-2-yl)ethanol

CAS Registry Number 1000521-75-4

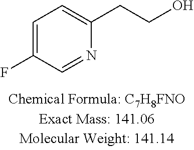

This compound was prepared in accordance with the procedure described in Publication No. US 2010/0331341 to Andersson et al., which is hereby incorporated by reference in its entirety.

### b) 2-(2-Chloroethyl)-5-fluoropyridine

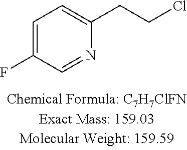

A solution of 2-(5-fluoropyridin-2-yl)ethanol (3.46 g, 24.5 mmol) in anhydrous tetrahydrofuran (13 mL) was treated with thionyl chloride (2.7 mL, 37 mmol) slowly. The reaction was heated to reflux and stirred for 16 h. After cooling to rt, the reaction mixture was concentrated, and the residue was partitioned between ethyl acetate and saturated sodium bicarbonate. The aqueous phase was extracted with additional ethyl acetate (2×), and the combined organics were washed with brine, dried over sodium sulfate, filtered, and concentrated. Flash chromatography (80 g ISCO Gold column, 5%-40% ethyl acetate/hexanes) provided the title compound (2.05 g, 52%) as an off-white solid: ¹H NMR (500 MHz, CDCl₃) δ 8.42 (d, J=3.0 Hz, 1H), 7.35 (td, J=8.5, 3.0 Hz, 1H), 7.21 (dd, J=8.5, 4.5 Hz, 1H), 3.90 (t, J=6.8 Hz, 2H), 3.22 (t, J=6.8 Hz, 2H); ESI MS m/z 160 [M+H]⁺.

### c) 4-(2-(5-Fluoropyridin-2-yl)ethyl)piperazin-2-one

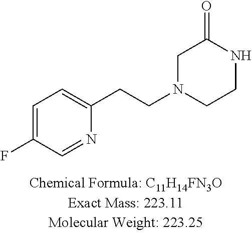 [SMILES: O=C1CN(CCc2ccc(F)cn2)CCN1]

A mixture of 2-(2-chloroethyl)-5-fluoropyridine (2.04 g, 12.8 mmol) and piperazinone (1.32 g, 12.8 mmol) in diisopropylethylamine (4.5 mL) was heated to reflux and stirred for 16 h. The reaction mixture was concentrated and purified by flash chromatography (120 g ISCO Gold column; 12%-100% B method, A=CH₂Cl₂, B=80:18:2 CH₂Cl₂/MeOH/NH₄OH), but the product still contained residual diisopropylethylamine. The material was partitioned between water and 90:9:1 CH₂Cl₂/MeOH/NH₄OH, the aqueous phase was extracted with additional 90:9:1 CH₂Cl₂/MeOH/NH₄OH (4×), and the combined organic extracts were dried over sodium sulfate, filtered, and concentrated to provide the title compound (1.13 g, 40%) as a white solid: ¹H NMR (500 MHz, CD₃OD) δ 8.35 (d, J=3.0 Hz, 1H), 7.54 (td, J=8.5, 3.0 Hz, 1H), 7.39 (dd, J=9.0, 4.5 Hz, 1H), 3.29 (t, J=6.0 Hz, 2H), 3.15 (s, 2H), 3.00 (t, J=7.5 Hz, 2H), 2.82 (t, J=7.5 Hz, 2H), 2.74 (t, J=5.5 Hz, 2H); ESI MS m/z 224 [M+H]⁺.

### d) tert-Butyl 7-(4-(2-(5-fluoropyridin-2-yl)ethyl)-2-oxopiperazin-1-yl)-3,4-dihydrobenzofuro[3,2-c]pyridine-2(1H)-carboxylate

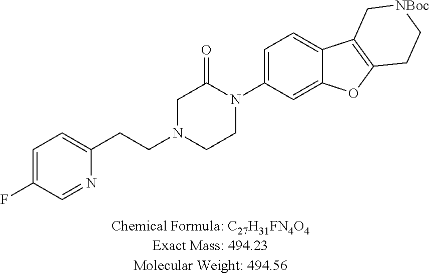 [SMILES: CC(C)(C)OC(=O)N1CCc2oc3cc(N4CCN(CCc5ccc(F)cn5)CC4=O)ccc3c2C1]

tert-Butyl 7-bromo-3,4-dihydrobenzofuro[3,2-c]pyridine-2(1H)-carboxylate (264 mg, 0.749 mmol) and 4-(2-(5-fluoropyridin-2-yl)ethyl)piperazin-2-one (167 mg, 0.748 mmol) were reacted according to Example 3 (step b) to provide the title compound (233 mg, 63%) as a white solid: ¹H NMR (500 MHz, CDCl₃) δ 8.40 (d, J=3.0 Hz, 1H), 7.41 (d, J=8.5 Hz, 1H), 7.37 (d, J=1.5 Hz, 1H), 7.35 (td, J=8.3, 3.0 Hz, 1H), 7.21 (dd, J=8.5, 4.5 Hz, 1H), 7.14 (d, J=8.0 Hz, 1H), 4.54 (s, 2H), 3.82 (s, 2H), 3.72 (t, J=5.3 Hz, 2H), 3.41 (s, 2H), 3.03 (t, J=7.5 Hz, 2H), 2.92-2.89 (m, 4H), 2.85 (s, 2H), 1.50 (s, 9H); ESI MS m/z 495 [M+H]⁺.

### e) 4-(2-(5-Fluoropyridin-2-yl)ethyl)-1-(1,2,3,4-tetrahydrobenzofuro[3,2-c]pyridin-7-yl)piperazin-2-one

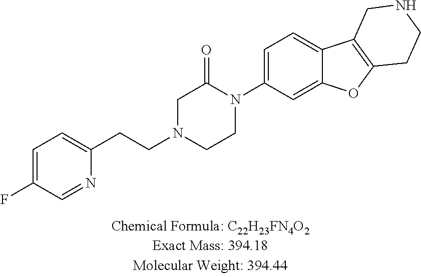 [SMILES: O=C1CN(CCc2ccc(F)cn2)CCN1c1ccc2c3c(oc2c1)CCNC3]

A solution of tert-butyl 7-(4-(2-(5-fluoropyridin-2-yl)ethyl)-2-oxopiperazin-1-yl)-3,4-dihydrobenzofuro[3,2-c]pyridine-2(1H)-carboxylate (232 mg, 0.469 mmol) in MeOH (5.0 mL) was treated with 2 N HCl in Et₂O (10 mL), and the resulting solution was stirred at ambient temperature for 16 h and then concentrated in vacuo. The resultant HCl salt was converted to the corresponding free base using an SCX-2 cartridge to provide the title compound (211 mg, quant. yield) as an off-white solid: ¹H NMR (500 MHz, DMSO-d₆) δ 8.48 (d, J=3.0 Hz, 1H), 7.65 (td, J=8.8, 3.0 Hz, 1H), 7.61 (d, J=2.0 Hz, 1H), 7.59 (d, J=8.0 Hz, 1H), 7.43 (dd, J=9.0, 4.5 Hz, 1H), 7.25 (dd, J=8.5, 2.0 Hz, 1H), 4.31 (s, 2H), 3.68 (t, J=5.3 Hz, 2H), 3.53 (t, J=6.3 Hz, 2H), 3.26 (s, 2H), 3.08 (t, J=5.8 Hz, 2H), 2.98 (t, J=7.5 Hz, 2H), 2.88-2.86 (m, 2H), 2.81 (t, J=7.3 Hz, 2H); ESI MS m/z 395 [M+H]⁺.

### f) 4-(2-(5-Fluoropyridin-2-yl)ethyl)-1-(1,2,3,4-tetrahydrobenzofuro[3,2-c]pyridin-7-yl)piperazin-2-one hydrochloride

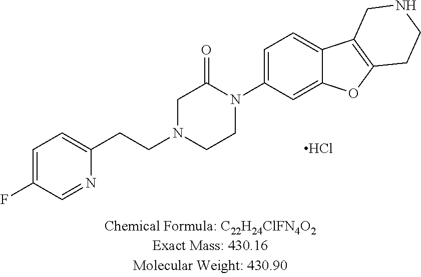 [SMILES: O=C1CN(CCc2ccc(F)cn2)CCN1c1ccc2c3c(oc2c1)CCNC3 ; Cl]

A solution of 4-(2-(5-fluoropyridin-2-yl)ethyl)-1-(1,2,3,4-tetrahydrobenzofuro[3,2-c]pyridin-7-yl)piperazin-2-one (207 mg, 0.525 mmol) in MeOH (5.0 mL) was treated with 2 N HCl in Et₂O (0.26 mL, 0.52 mmol), and the resulting suspension was concentrated. The solid was dissolved in H₂O (3 mL), frozen, and lyophilized overnight to provide the title compound (230 mg, quant. yield) as a white solid: mp 203-209° C.; ¹H NMR (500 MHz, CD₃OD) δ 8.64 (d, J=2.0 Hz, 1H), 7.87 (td, J=8.5, 3.0 Hz, 1H), 7.69 (dd, J=8.5, 4.5 Hz, 1H), 7.63-7.62 (m, 2H), 7.34 (dd, J=8.3, 1.8 Hz, 1H), 4.45 (s, 2H), 4.27 (s, 2H), 4.13 (t, J=5.5 Hz, 2H), 3.95 (t, J=5.5 Hz, 2H), 3.84 (t, J=7.3 Hz, 2H), 3.69 (t, J=6.3 Hz, 2H), 3.51 (t, J=7.3 Hz, 2H), 3.20 (t, J=6.0 Hz, 2H); ESI MS m/z 395 [M+H]⁺; HPLC (Method A)>99% (AUC), t_{R}=9.7 min.

### Example 71

### Preparation of 1-(1,2,3,4-Tetrahydrobenzofuro[3,2-c]pyridin-7-yl)-4-(4-(trifluoromethyl)phenyl)pyridin-2(1H)-one hydrochloride

### a) 4-(4-(Trifluoromethyl)phenyl)pyridin-2(1H)-one

CAS Registry Number 942947-10-6

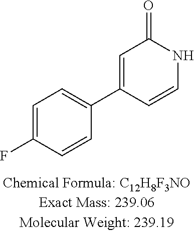 [SMILES: O=c1cc(-c2ccc(F)cc2)cc[nH]1]

This compound was prepared in accordance with the procedure described in PCT Publication No. WO 2011/003005 to Guzzo et al., which is hereby incorporated by reference in its entirety.

### b) tert-Butyl 7-(2-oxo-4-(4-(trifluoromethyl)phenyl)pyridin-1(2H)-yl)-3,4-dihydrobenzofuro[3,2-c]pyridine-2(1H)-carboxylate

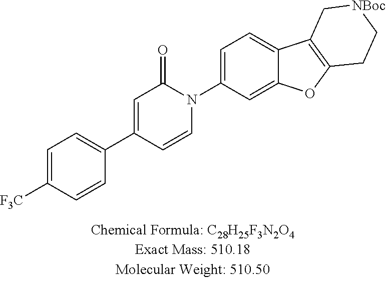 [SMILES: CC(C)(C)OC(=O)N1CCc2oc3cc(-n4ccc(-c5ccc(C(F)(F)F)cc5)cc4=O)ccc3c2C1]

tert-butyl 7-bromo-3,4-dihydrobenzofuro[3,2-c]pyridine-2(1H)-carboxylate (195 mg, 0.554 mmol) and 4-(4-(trifluoromethyl)phenyl)pyridin-2(1H)-one (132 mg, 0.552 mmol) were reacted according to Example 3 (step b) to provide the title compound (215 mg, 77%) as an off-white solid: ¹H NMR (500 MHz, CDCl₃) δ 7.77-7.73 (m, 4H), 7.54 (d, J=1.5 Hz, 1H), 7.52 (s, 1H), 7.50 (d, J=7.0 Hz, 1H), 7.28 (br s, 1H), 6.91 (d, J=2.0 Hz, 1H), 6.51 (dd, J=7.5, 1.8 Hz, 1H), 4.59 (s, 2H), 3.85 (s, 2H), 2.89 (s, 2H), 1.52 (s, 9H); ESI MS m/z 511 [M+H]⁺.

### c) 1-(1,2,3,4-Tetrahydrobenzofuro[3,2-c]pyridin-7-yl)-4-(4-(trifluoromethyl)phenyl)pyridin-2(1H)-one

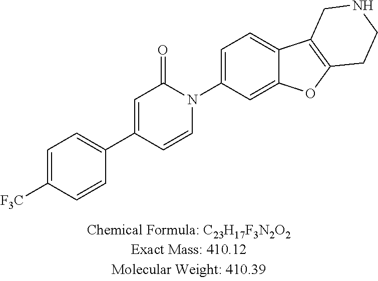 [SMILES: O=c1cc(-c2ccc(C(F)(F)F)cc2)ccn1-c1ccc2c3c(oc2c1)CCNC3]

A solution of tert-butyl 7-(2-oxo-4-(4-(trifluoromethyl)phenyl)pyridin-1(2H)-yl)-3,4-dihydrobenzofuro[3,2-c]pyridine-2(1H)-carboxylate (184 mg, 0.360 mmol) in MeOH (5.0 mL) was treated with 2 N HCl in Et₂O (10 mL), and the resulting solution was stirred at ambient temperature for 16 h and then concentrated in vacuo. The resultant HCl salt was converted to the corresponding free base using an SCX-2 cartridge to provide the title compound (145 mg, 98%) as an off-white solid: ¹H NMR (500 MHz, CD₃OD) δ 7.76-7.73 (m, 4H), 7.52 (d, J=2.0 Hz, 1H), 7.51 (d, J=7.0 Hz, 1H), 7.48 (d, J=8.0 Hz, 1H), 7.26-7.24 (m, 1H), 6.91 (d, J=1.5 Hz, 1H), 6.52 (dd, J=7.0, 2.0 Hz, 1H), 4.01 (t, J=2.0 Hz, 2H), 3.27 (t, J=5.8 Hz, 2H), 2.84-2.82 (m, 2H); ESI MS m/z 411 [M+H]⁺.

### d) 1-(1,2,3,4-Tetrahydrobenzofuro[3,2-c]pyridin-7-yl)-4-(4-(trifluoromethyl)phenyl)pyridin-2(1H)-one hydrochloride

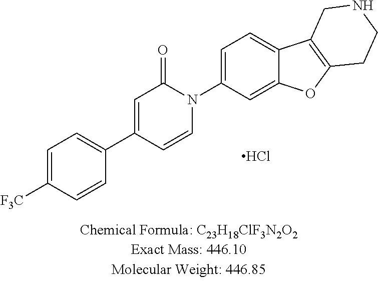 [SMILES: O=c1cc(-c2ccc(C(F)(F)F)cc2)ccn1-c1ccc2c3c(oc2c1)CCNC3 ; Cl]

A solution of 1-(1,2,3,4-tetrahydrobenzofuro[3,2-c]pyridin-7-yl)-4-(4-(trifluoromethyl)phenyl)pyridin-2(1H)-one (144 mg, 0.351 mmol) in MeOH (5.0 mL) was treated with 2 N HCl in Et₂O (0.18 mL, 0.35 mmol), and the resulting suspension was concentrated. The solid was dissolved in H₂O (3 mL) and MeOH (0.7 mL), frozen, and lyophilized overnight to provide the title compound (156 mg, 99%) as an off-white solid: mp>300° C.; ¹H NMR (500 MHz, DMSO-d₆) δ 9.46 (s, 2H), 8.02 (d, J=8.0 Hz, 2H), 7.88 (d, J=8.0 Hz, 2H), 7.84 (d, J=7.0 Hz, 1H), 7.81 (d, J=1.5 Hz, 1H), 7.74 (d, J=8.0 Hz, 1H), 7.38 (dd, J=8.3, 1.8 Hz, 1H), 6.90 (d, J=1.5 Hz, 1H), 6.75 (dd, J=7.5, 2.0 Hz, 1H), 4.38 (s, 2H), 3.57 (t, J=6.0 Hz, 2H), 3.13 (t, J=6.0 Hz, 2H); ESI MS m/z 411 [M+H]⁺; HPLC (Method A)>99% (AUC), t_{R}=14.7 min.

### Example 72

### Preparation of 1-(2-(2-Fluoroethyl)-1,2,3,4-tetrahydrobenzofuro[3,2-c]pyridin-7-yl)-4-((5-fluoropyridin-2-yl)methoxy)pyridin-2(1H)-one hydrochloride

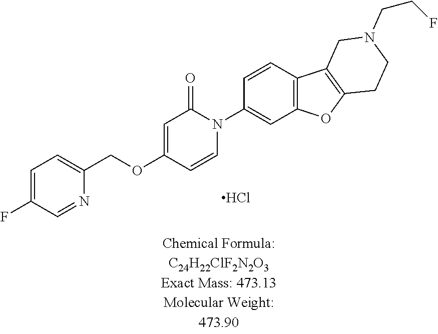 [SMILES: O=c1cc(OCc2ccc(F)cn2)ccn1-c1ccc2c3c(oc2c1)CCN(CCF)C3 ; Cl]

4-((5-Fluoropyridin-2-yl)methoxy)-1-(1,2,3,4-tetrahydrobenzofuro[3,2-c]pyridin-7-yl)pyridin-2(1H)-one (100 mg, 0.229 mmol) was alkylated according to Example 59 (step a) and converted to the hydrochloride according to Example 12 (step d) to provide the title compound (95 mg, 88%) as a white solid: ¹H NMR (300 MHz, CD₃OD) δ 8.52 (d, J=2.6, Hz, 1H), 7.79-7.56 (m, 5H), 7.30 (dd, J=8.3, 1.8 Hz, 1H), 6.34 (dd, J=7.7, 2.7 Hz, 1H), 6.13 (d, J=2.7 Hz, 1H), 5.27 (s, 2H), 5.07 (t, J=4.4 Hz, 1H), 4.96-4.86 (m, 1H), 4.86-4.77 (m, 1H), 4.60-4.50 (m, 1H), 4.04-3.94 (m, 1H), 3.89-3.84 (m, 1H), 3.81-3.75 (m, 2H), 3.41-3.23 (m, 2H); ESI MS m/z 438 [M+H]⁺; HPLC (Method A)>99% (AUC), t_{R}=12.6 min.

### Example 73

### Preparation of 4-(Benzyloxy)-1-(1,2,3,5,6,11c-hexahydrobenzofuro[2,3-g]indolizin-9-yl)pyridin-2(1H)-one hydrochloride

### a) 9-Bromo-1,2,3,5,6,11c-hexahydrobenzofuro[2,3-g]indolizine

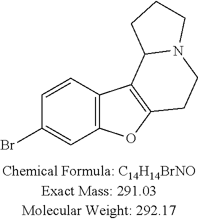 [SMILES: Brc1ccc2c3c(oc2c1)CCN1CCCC31]

O-(3-bromophenyl)hydroxylamine hydrochloride (1.95 g, 8.67 mmol) and hexahydroindolizin-7(1H)-one (1.20 g, 8.62 mmol) were reacted according to Example 10 (step b) to provide the title compound (255 mg, 10%) as a white foam: ESI MS m/z 292/294 [M+H]⁺.

### b) 4-(Benzyloxy)-1-(1,2,3,5,6,11c-hexahydrobenzofuro[2,3-g]indolizin-9-yl)pyridin-2(1H)-one hydrochloride

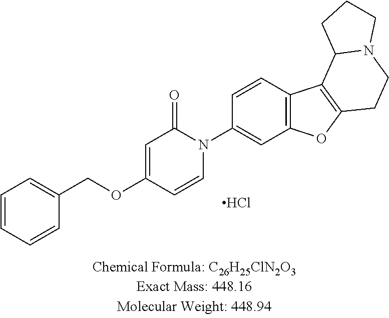 [SMILES: O=c1cc(OCc2ccccc2)ccn1-c1ccc2c3c(oc2c1)CCN1CCCC31 ; Cl]

4-(benzyloxy)pyridin-2(1H)-one (123 mg, 0.611 mmol) and 9-bromo-1,2,3,5,6,11c-hexahydrobenzofuro[2,3-g]indolizine (125 mg, 0.428 mmol) were reacted and the product converted to the hydrochloride according to Example 12 to provide the title compound (66 mg, 34%) as a white solid: ¹H NMR (300 MHz, CD₃OD) δ 7.70 (d, J=8.3 Hz, 1H), 7.67-7.57 (m, 2H), 7.52-7.26 (m, 6H), 6.41-6.31 (m, 1H), 6.16 (d, J=2.7 Hz, 1H), 5.20 (s, 2H), 5.13 (t, J=6.9 Hz, 1H), 3.84-3.67 (m, 3H), 3.56-3.40 (m, 1H), 3.30-3.14 (m, 2H), 2.87-2.61 (m, 1H), 2.40-2.11 (m, 3H); ESI MS m/z 413 [M+H]⁺; HPLC (Method A) 98.8% (AUC), t_{R}=14.3 min.

### Example 74

### Preparation of 4-(Benzyloxy)-1-(1,2,3,4-tetrahydrobenzofuro[3,2-c]pyridin-8-yl)pyridin-2(1H)-one hydrochloride

### a) tert-Butyl 8-(4-(benzyloxy)-2-oxopyridin-1(2H)-yl)-3,4-dihydrobenzofuro[3,2-c]pyridine-2(1H)-carboxylate

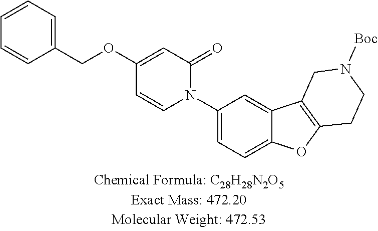 [SMILES: CC(C)(C)OC(=O)N1CCc2oc3ccc(-n4ccc(OCc5ccccc5)cc4=O)cc3c2C1]

A mixture of tert-butyl 8-bromo-3,4-dihydrobenzofuro[3,2-c]pyridine-2(1H)-carboxylate (0.30 g, 0.85 mmol), 4-benzyloxy pyridinone (0.17 g, 0.85 mmol) and K₂CO₃ (0.13 g, 0.96 mmol) in DMSO (10 mL) was degassed with N₂. The suspension was treated with 8-hydroxy quinolone (38 mg, 0.26 mmol) and purged with N₂ for 5 min. CuI (83 mg, 0.43 mmol) was added, and the resulting suspension was heated at 120° C. under N₂ for 18 h. The reaction mixture was cooled, diluted with H₂O (20 mL) and stirred for 10 min. The solution was diluted with EtOAc (30 mL), and the resulting layers were separated. The aqueous phase was extracted with EtOAc (2×10 mL), and the combined organic extracts were washed with water followed by brine and concentrated. The crude product was purified by flash chromatography (silica gel, Hexanes/EtOAc, 100:0 to 70:30) to afford the title compound (0.10 g, 25%) as an off-white solid: ¹H NMR (300 MHz, CDCl₃) δ 7.50 (d, J=8.7 Hz, 1H), 7.46-7.35 (m, 6H), 7.30-7.22 (m, 1H), 7.20 (dd, J=8.7, 2.1 Hz, 1H), 6.11-6.01 (m, 2H), 5.05 (s, 2H), 4.53 (s, 2H), 3.90-3.77 (m, 2H), 2.93-2.81 (m, 2H), 1.49 (s, 9H); APCI MS m/z 473 [M+H]⁺.

### b) 4-(Benzyloxy)-1-(1,2,3,4-tetrahydrobenzofuro[3,2-c]pyridin-8-yl)pyridin-2(1H)-one hydrochloride

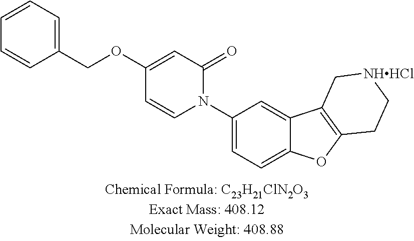

A solution of tert-butyl 8-(4-(benzyloxy)-2-oxopyridin-1(2H)-yl)-3,4-dihydrobenzofuro[3,2-c]pyridine-2(1H)-carboxylate (0.10 g, 0.21 mmol) in THF (4 mL) was cooled to 0° C. and treated with conc. HCl (2 mL). The resulting solution was stirred at ambient temperature for 18 h and then concentrated under reduced pressure. The residue was diluted with H₂O (10 mL), and the resulting solution was treated with 10% NaOH solution until the solution was basic. The aqueous solution was extracted with CH₂Cl₂ (3×15 mL). The combined organic extracts were dried over Na₂SO₄, filtered and concentrated to dryness. The crude product was purified by flash column chromatography (silica gel, CH₂Cl₂/MeOH, 100:0 to 90:10) to provide the free base which was dissolved in CH₂Cl₂ (2.0 mL) and treated with 2 N HCl in Et₂O at 0° C. After stirring at 0° C. for 20 min, the resulting solid was filtered, washed with MTBE and lyophilized from H₂O to provide the title compound (82 mg, 94%) as an off white solid: ¹H NMR (300 MHz, CD₃OD) δ 7.77 (d, J=7.5 Hz, 1H), 7.68-7.60 (m, 2H), 7.52-7.31 (m, 6H), 6.55 (d, J=7.2 Hz, 1H), 6.31 (br s, 1H), 5.26 (s, 2H), 4.44 (s, 2H), 3.70 (t, J=6.0 Hz, 2H), 3.22 (t, J=5.7 Hz, 2H); APCI MS m/z 373 [M+H]⁺; HPLC (Method C)>99% (AUC), t_{R}=20.51 min.

### Example 75

### Preparation of 4-((5-Fluoropyridin-2-yl)methoxy)-1-(2,3,4,5-tetrahydro-1H-benzofuro[3,2-c]azepin-9-yl)pyridin-2(1H)-one hydrochloride

### a) tert-Butyl 9-(4-((5-fluoropyridin-2-yl)methoxy)-2-oxopyridin-1(2H)-yl)-4,5-dihydro-1H-benzofuro[3,2-c]azepine-2(3H)-carboxylate

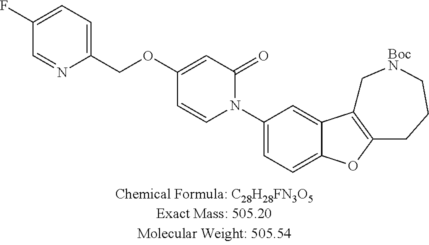 [SMILES: CC(C)(C)OC(=O)N1CCCc2oc3ccc(-n4ccc(OCc5ccc(F)cn5)cc4=O)cc3c2C1]

tert-Butyl 9-bromo-4,5-dihydro-1H-benzofuro[3,2-c]azepine-2(3H)-carboxylate (0.30 g, 0.81 mmol) and 4-((5-fluoropyridin-2-yl)methoxy)pyridin-2(1H)-one (179 mg, 0.813 mmol) were reacted according to Example 74 (step a) to provide the title compound (85 mg, 20%) as an off-white solid: ¹H NMR (400 MHz, CDCl₃) δ 8.49 (d, J=2.4 Hz, 1H), 7.53-7.35 (m, 4H), 7.32-7.23 (m, 1H), 7.17 (d, J=8.0 Hz, 1H), 6.12-6.03 (m, 2H), 5.16 (s, 2H), 4.61-4.47 (m, 2H), 3.75-3.61 (m, 2H), 3.02 (t, J=6.0 Hz, 2H), 2.07-1.95 (m, 2H), 1.49-1.34 (m, 9H); APCI MS m/z 506 [M+H]⁺.

### b) 4-((5-Fluoropyridin-2-yl)methoxy)-1-(2,3,4,5-tetrahydro-1H-benzofuro[3,2-c]azepin-9-yl)pyridin-2(1H)-one hydrochloride

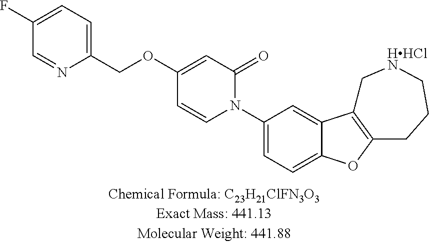

tert-Butyl 9-(4-((5-fluoropyridin-2-yl)methoxy)-2-oxopyridin-1(2H)-yl)-4,5-dihydro-1H-benzofuro[3,2-c]azepine-2(3H)-carboxylate (85 mg, 0.16 mmol) and conc. HCl (0.5 mL) were reacted according to Example 74 (step b) to provide the title compound (45 mg, 60%) as an off white solid: ¹H NMR (300 MHz, DMSO-d₆) δ 9.64-9.51 (m, 2H), 8.62 (d, J=2.7 Hz, 1H), 7.89-7.78 (m, 1H), 7.75 (d, J=1.5 Hz, 1H), 7.71-7.54 (m, 3H), 7.25 (dd, J=8.7, 1.5 Hz, 1H), 6.16 (dd, J=7.5, 2.4 Hz, 1H), 5.99 (d, J=2.4 Hz, 1H), 5.22 (s, 2H), 4.36 (br s, 2H), 3.52-3.38 (m, 2H), 3.18-3.03 (m, 2H), 2.18-2.02 (m, 2H); APCI MS m/z 406 [M+H]⁺; HPLC (Method C) 98.5% (AUC), t_{R}=19.54 min.

### Example 76

### Preparation of 4-((5-Fluoropyridin-2-yl)methoxy)-1-(1,2,3,4-tetrahydrobenzofuro[3,2-c]pyridin-8-yl)pyridin-2(1H)-one hydrochloride

### a) tert-Butyl 8-(4-((5-fluoropyridin-2-yl)methoxy)-2-oxopyridin-1(2H)-yl)-3,4-dihydrobenzofuro[3,2-c]pyridine-2(1H)-carboxylate

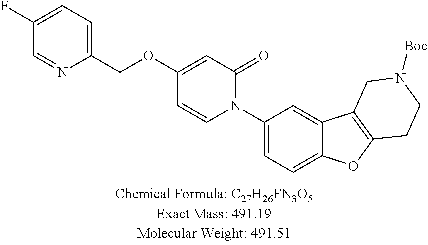 [SMILES: CC(C)(C)OC(=O)N1CCc2oc3ccc(-n4ccc(OCc5ccc(F)cn5)cc4=O)cc3c2C1]

tert-Butyl 8-bromo-3,4-dihydrobenzofuro[3,2-c]pyridine-2(1H)-carboxylate (0.30 g, 0.85 mmol) and 4-((5-fluoropyridin-2-yl)methoxy)pyridin-2(1H)-one (0.18 g, 0.85 mmol) were reacted according to Example 74 (step a) to provide the title compound (85 mg, 20%) as an off-white solid: ¹H NMR (400 MHz, CDCl₃) δ 8.49 (d, J=2.4 Hz, 1H), 7.53-7.35 (m, 4H), 7.29 (d, J=7.2 Hz, 1H), 7.18 (dd, J=7.6, 2.0 Hz, 1H), 6.10 (dd, J=7.6, 2.8 Hz, 1H), 6.06 (d, J=2.8 Hz, 1H), 5.17 (s, 2H), 4.53 (s, 2H), 3.89-3.77 (m, 2H), 2.93-2.81 (m, 2H), 1.49 (s, 9H); APCI MS m/z 492 [M+H]⁺.

### b) 4-((5-Fluoropyridin-2-yl)methoxy)-1-(1,2,3,4-tetrahydrobenzofuro[3,2-c]pyridin-8-yl)pyridin-2(1H)-one hydrochloride

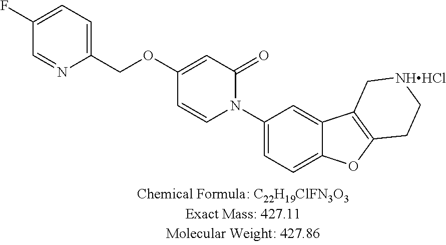

tert-Butyl 8-(4-((5-fluoropyridin-2-yl)methoxy)-2-oxopyridin-1(2H)-yl)-3,4-dihydrobenzofuro[3,2-c]pyridine-2(1H)-carboxylate (85 mg, 0.21 mmol) and conc. HCl (2 mL) were reacted according to Example 74 (step b) to provide the title compound (68 mg, 91%) as an off white solid: ¹H NMR (300 MHz, DMSO-d₆) δ 9.93-9.81 (m, 2H), 8.62 (d, J=2.7 Hz, 1H), 7.89-7.78 (m, 1H), 7.73-7.57 (m, 4H), 7.29 (dd, J=8.7 Hz, 1.8 Hz, 1H), 6.16 (dd, J=7.8 Hz, 2.4 Hz, 1H), 6.00 (d, J=2.4 Hz, 1H), 5.22 (s, 2H), 4.28 (s, 2H), 3.59-3.47 (m, 2H), 3.18-3.08 (m, 2H); APCI MS m/z 392 [M+H]⁺; HPLC (Method C)>99% (AUC), t_{R}=19.2 min.

### Example 77

### Preparation of 4-(Benzyloxy)-1-(2,3,4,5-tetrahydro-1H-benzofuro[2,3-d]azepin-9-yl)pyridin-2(1H)-one hydrochloride

### a) O-(4-Bromophenyl)hydroxylamine hydrochloride

CAS Registry Number 65440-82-6

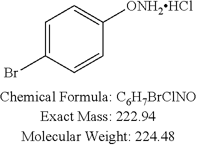

4-bromophenol (44.0 g, 254 mmol) and hydroxylamine-O-sulfonic acid (7.18 g, 63.58 mmol) were reacted according to Example 2 (step a) to provide the title compound (8.0 g, 56%) as a brown solid: ¹H NMR (300 MHz, DMSO-d₆) δ 9.95 (br s, 3H), 7.59-7.51 (m, 2H), 7.23-7.15 (m, 2H).

### b) tert-Butyl 9-bromo-4,5-dihydro-1H-benzofuro[2,3-d]azepine-3(2H)-carboxylate

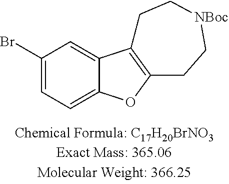 [SMILES: CC(C)(C)OC(=O)N1CCc2oc3ccc(Br)cc3c2CC1]

O-(4-bromophenyl)hydroxylamine hydrochloride (2.0 g, 8.9 mmol) and tert-butyl 4-oxoazepane-1-carboxylate (2.28 g, 10.69 mmol) were reacted according to Example 2 (step b) to provide the title compound (320 mg, 9%, minor) as an off-white solid: ¹H NMR (300 MHz, CDCl₃) δ 7.51 (s, 1H), 7.31 (dd, J=8.7 Hz, 1.8 Hz, 1H), 7.24 (d, J=8.7 Hz, 1H), 3.74-3.61 (m, 4H), 3.15-3.01 (m, 2H), 2.87-2.73 (m, 2H), 1.48 (s, 9H); and tert-butyl 9-bromo-4,5-dihydro-1H-benzofuro[3,2-c]azepine-2(3H)-carboxylate (1.1 g, 35%, major) as an off-white solid: ¹H NMR (300 MHz, CDCl₃) δ 7.63-7.48 (m, 1H), 7.35-7.17 (m, 2H), 4.63-4.40 (m, 2H), 3.77-3.57 (m, 2H), 2.99 (t, J=6.3 Hz, 2H), 2.05-1.92 (m, 2H), 1.51-1.32 (m, 9H).

### c) tert-Butyl 9-(4-(benzyloxy)-2-oxopyridin-1(2H)-yl)-4,5-dihydro-1H-benzofuro[2,3-d]azepine-3(2H)-carboxylate

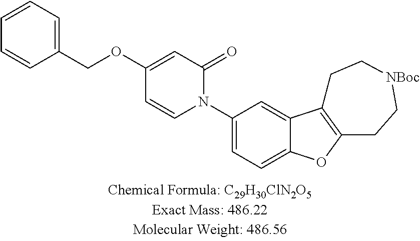 [SMILES: CC(C)(C)OC(=O)N1CCc2oc3ccc(-n4ccc(OCc5ccccc5)cc4=O)cc3c2CC1]

tert-Butyl 9-bromo-4,5-dihydro-1H-benzofuro[2,3-d]azepine-3(2H)-carboxylate (150 mg, 0.42 mmol) and 4-benzyloxy pyridinone (85 mg, 0.42 mmol) were reacted according to Example 74 (step a) to provide the title compound (60 mg, 30%) as an off-white solid: ¹H NMR (400 MHz, CDCl₃) δ 7.47-7.33 (m, 7H), 7.29-7.23 (m, 1H), 7.19-7.11 (m, 1H), 6.11-6.02 (m, 2H), 5.05 (s, 2H), 3.67 (t, J=5.6 Hz, 4H), 3.17-3.04 (m, 2H), 2.88-2.76 (m, 2H), 1.48 (s, 9H); APCI MS m/z 487 [M+H]⁺.

### d) 4-(Benzyloxy)-1-(2,3,4,5-tetrahydro-1H-benzofuro[2,3-d]azepin-9-yl)pyridin-2(1H)-one hydrochloride

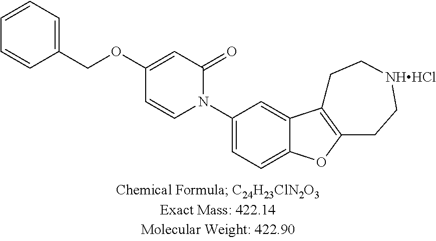

tert-Butyl 9-(4-(benzyloxy)-2-oxopyridin-1(2H)-yl)-4,5-dihydro-1H-benzofuro[2,3-d]azepine-3(2H)-carboxylate (60 mg, 0.12 mmol) and Cone. HCl (0.4 mL) were reacted according to Example 74 (step b) to provide the title compound (50 mg, 96%) as an off white solid: ¹H NMR (300 MHz, DMSO-d₆) δ 9.55-9.43 (m, 2H), 7.64 (m, 3H), 7.51-7.33 (m, 5H), 7.22 (dd, J=8.4, 2.1 Hz, 1H), 6.11 (dd, J=7.5, 2.7 Hz, 1H), 5.98 (d, J=2.7 Hz, 1H), 5.16 (s, 2H), 3.47-3.23 (m, 6H), 3.12-3.00 (m, 2H); APCI MS m/z 387 [M+H]⁺; HPLC (Method C) 98.3% (AUC), t_{R}=20.76 min.

### Example 78

### Preparation of 4-(Benzyloxy)-1-(2,3,4,5-tetrahydro-1H-benzofuro[3,2-c]azepin-9-yl)pyridin-2(1H)-one hydrochloride

### a) tert-Butyl 9-bromo-4,5-dihydro-1H-benzofuro[3,2-c]azepine-2(3H)-carboxylate

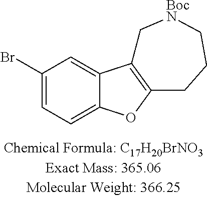 [SMILES: CC(C)(C)OC(=O)N1CCCc2oc3ccc(Br)cc3c2C1]

The title compound was isolated from the reaction described in Example 77 (step b): ¹H NMR (300 MHz, CDCl₃) δ 7.63-7.48 (m, 1H), 7.35-7.17 (m, 2H), 4.63-4.40 (m, 2H), 3.77-3.57 (m, 2H), 2.99 (t, J=6.3 Hz, 2H), 2.05-1.92 (m, 2H), 1.51-1.32 (m, 9H).

### a) tert-Butyl 9-(4-(benzyloxy)-2-oxopyridin-1(2H)-yl)-4,5-dihydro-1H-benzofuro[3,2-c]azepine-2(3H)-carboxylate

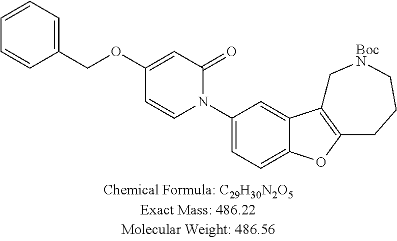 [SMILES: CC(C)(C)OC(=O)N1CCCc2oc3ccc(-n4ccc(OCc5ccccc5)cc4=O)cc3c2C1]

tert-Butyl 9-bromo-4,5-dihydro-1H-benzofuro[3,2-c]azepine-2(3H)-carboxylate (0.30 g, 0.81 mmol) and 4-benzyloxy pyridinone (164 mg, 0.815 mmol) were reacted according to Example 74 (step a) to provide the title compound (130 mg, 32%) as an off-white solid: ¹H NMR (300 MHz, CDCl₃) δ 7.48-7.34 (m, 7H), 7.29-7.22 (m, 1H), 7.17 (dd, J=8.7, 2.1 Hz, 1H), 6.12-6.01 (m, 2H), 5.05 (s, 2H), 4.63-4.45 (m, 2H), 3.75-3.60 (m, 2H), 3.01 (t, J=6.3 Hz, 2H), 2.08-1.93 (m, 2H), 1.51-1.32 (m, 9H); APCI MS m/z 487 [M+H]⁺.

### b) 4-(Benzyloxy)-1-(2,3,4,5-tetrahydro-1H-benzofuro[3,2-c]azepin-9-yl)pyridin-2(1H)-one hydrochloride

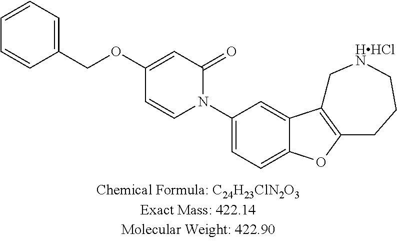

tert-Butyl 9-(4-(benzyloxy)-2-oxopyridin-1(2H)-yl)-4,5-dihydro-1H-benzofuro[3,2-c]azepine-2(3H)-carboxylate (130 mg, 0.26 mmol) and conc. HCl (1.0 mL) were reacted according to Example 74 (step b) to provide the title compound (110 mg, 97%) as a white solid: ¹H NMR (400 MHz, DMSO-d₆) δ 9.46-9.36 (m, 2H), 7.75 (d, J=2.0 Hz, 1H), 7.66-7.54 (m, 2H), 7.51-7.34 (m, 5H), 7.25 (dd, J=8.4, 2.0 Hz, 1H), 6.12 (dd, J=7.6, 2.8 Hz, 1H), 5.99 (d, J=2.8 Hz, 1H), 5.15 (s, 2H), 4.41-4.33 (m, 2H), 3.52-3.41 (m, 2H), 3.10 (t, J=5.6 Hz, 2H), 2.15-2.02 (m, 2H); APCI MS m/z 387 [M+H]⁺; HPLC (Method C)>99% (AUC), t_{R}=20.45 min.

### Example 79

### Preparation of 4-((5-Fluoropyridin-2-yl)methoxy)-1-(2,3,4,5-tetrahydro-1H-benzofuro[2,3-d]azepin-9-yl)pyridin-2(1H)-one hydrochloride

### a) tert-Butyl 9-(4-((5-fluoropyridin-2-yl)methoxy)-2-oxopyridin-1(2H)-yl)-4,5-dihydro-1H-benzofuro[2,3-d]azepine-3(2H)-carboxylate

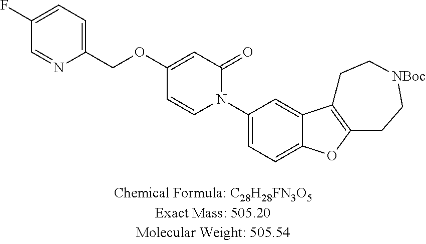 [SMILES: CC(C)(C)OC(=O)N1CCc2oc3ccc(-n4ccc(OCc5ccc(F)cn5)cc4=O)cc3c2CC1]

tert-Butyl 9-bromo-4,5-dihydro-1H-benzofuro[2,3-d]azepine-3(2H)-carboxylate (150 mg, 0.42 mmol) and 4-((5-fluoropyridin-2-yl)methoxy)pyridin-2(1H)-one (93 mg, 0.42 mmol) were reacted according to Example 74 (step a) to provide the title compound (65 mg, 32%) as an off-white solid: ¹H NMR (300 MHz, CDCl₃) δ 8.48 (br s, 1H), 7.54-7.40 (m, 3H), 7.37 (d, J=2.1 Hz, 1H), 7.32-7.23 (m, 1H), 7.19-7.10 (m, 1H), 6.12-6.03 (m, 2H), 5.16 (s, 2H), 3.77-3.60 (m, 4H), 3.19-3.02 (m, 2H), 2.89-2.75 (m, 2H), 1.48 (s, 9H); APCI MS m/z 506 [M+H]⁺.

### b) 4-((5-Fluoropyridin-2-yl)methoxy)-1-(2,3,4,5-tetrahydro-1H-benzofuro[2,3-d]azepin-9-yl)pyridin-2(1H)-one hydrochloride

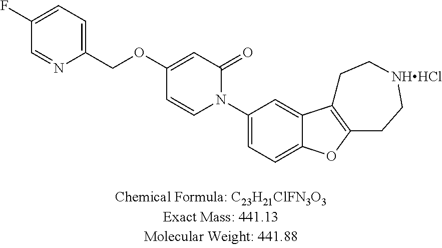

tert-Butyl 9-(4-((5-fluoropyridin-2-yl)methoxy)-2-oxopyridin-1(2H)-yl)-4,5-dihydro-1H-benzofuro[2,3-d]azepine-3(2H)-carboxylate (65 mg, 0.12 mmol) and cone. HCl (1.0 mL) were reacted according to Example 74 (step b) to provide the title compound (32 mg, 56%) as a brown solid: ¹H NMR (400 MHz, CD₃OD) δ 8.75 (s, 1H), 8.14-7.79 (m, 3H), 7.72-7.55 (m, 2H), 7.32 (d, J=8.0 Hz, 1H), 6.73-6.60 (m, 1H), 6.42 (br s, 1H), 5.48 (s, 2H), 3.63-3.49 (m, 4H), 3.44-3.35 (m, 2H), 3.22-3.10 (m, 2H); APCI MS m/z 406 [M+H]⁺; HPLC (Method C)>99% (AUC), t_{R}=19.50 min.

### Example 80

### Preparation of 4-((5-Fluoropyridin-2-yl)methoxy)-1-(1,2,3,5,6,11c-hexahydrobenzofuro[2,3-g]indolizin-9-yl)pyridin-2(1H)-one hydrochloride

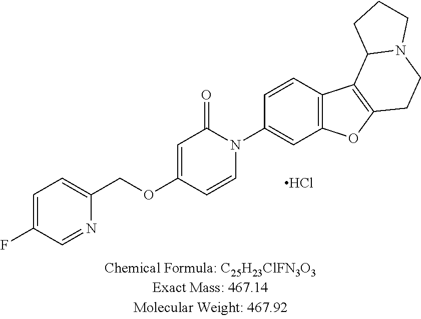 [SMILES: O=c1cc(OCc2ccc(F)cn2)ccn1-c1ccc2c3c(oc2c1)CCN1CCCC31 ; Cl]

4-((5-Fluoropyridin-2-yl)methoxy)pyridin-2(1H)-one (117 mg, 0.530 mmol) and 9-bromo-1,2,3,5,6,11c-hexahydrobenzofuro[2,3-g]indolizine (120 mg, 0.411 mmol) were reacted and the product converted to the hydrochloride according to Example 12 to provide the title compound (35 mg, 18%) as a white solid: ¹H NMR (300 MHz, CD₃OD) δ 8.62 (d, J=2.7 Hz, 1H), 7.93-7.61 (m, 5H), 7.32 (dd, J=8.3, 1.9 Hz, 1H), 6.43 (dd, J=7.6, 2.7 Hz, 1H), 6.21 (d, J=2.7 Hz, 1H), 5.34 (s, 2H), 5.15 (t, J=6.9 Hz, 1H), 3.84-3.67 (m, 3H), 3.55-3.40 (m, 1H), 3.30-3.21 (m, 2H), 2.70 (dt, J=14.4, 6.5 Hz, 1H), 2.38-2.14 (m, 3H); ESI MS m/z 432 [M+H]⁺; HPLC (Method A)>97.5% (AUC), t_{R}=12.8 min.

### Example 81

### Preparation of 1-(2-Isopropyl-2,3,4,5-tetrahydro-1H-benzofuro[3,2-c]azepin-8-yl)-4-((6-(trifluoromethyl)pyridin-2-yl)methoxy)pyridin-2(1H)-one hydrochloride

### a) 1-(2-Isopropyl-2,3,4,5-tetrahydro-1H-benzofuro[3,2-c]azepin-8-yl)-4-((6-(trifluoromethyl)pyridin-2-yl)methoxy)pyridin-2(1H)-one

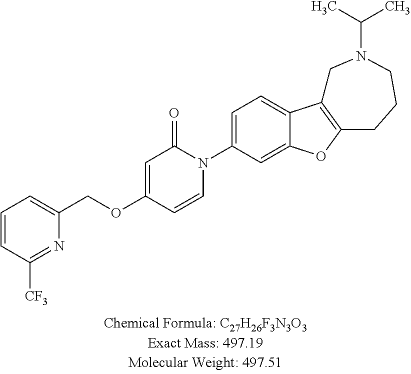 [SMILES: CC(C)N1CCCc2oc3cc(-n4ccc(OCc5cccc(C(F)(F)F)n5)cc4=O)ccc3c2C1]

A mixture of 1-(2,3,4,5-tetrahydro-1H-benzofuro[3,2-c]azepin-8-yl)-4-((6-(trifluoromethyl)pyridin-2-yl)methoxy)pyridin-2(1H)-one hydrochloride (43 mg, 0.087 mmol) in dichloromethane (2 mL) and acetone (2 mL) was treated with acetic acid (0.2 mL). After stirring the mixture at ambient temperature for 5 minutes, 2-picoline borane complex (37 mg, 0.35 mmol) was added. The reaction mixture was heated at 50° C. for 3 h. The mixture was cooled, diluted with saturated aqueous NaHCO₃ solution (50 mL) and extracted with dichloromethane (5×50 mL). The combined organic extracts were dried over Na₂SO₄ and concentrated under reduced pressure. Purification by flash chromatography (silica gel, CH₂Cl₂/(90:9:1 CH₂Cl₂/MeOH/NH₄OH), 100:0 to 25:75) afforded the title compound (38 mg, 87%) as a colorless solid: ESI MS m/z 498 [M+H]⁺.

### b) 1-(2-Isopropyl-2,3,4,5-tetrahydro-1H-benzofuro[3,2-c]azepin-8-yl)-4-((6-(trifluoromethyl)pyridin-2-yl)methoxy)pyridin-2(1H)-one hydrochloride

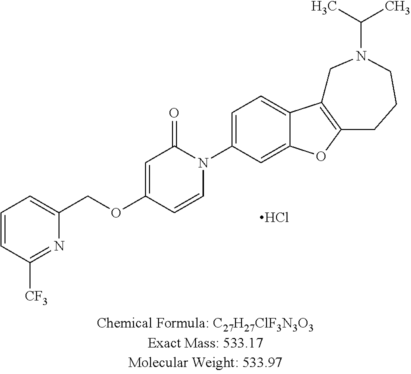 [SMILES: CC(C)N1CCCc2oc3cc(-n4ccc(OCc5cccc(C(F)(F)F)n5)cc4=O)ccc3c2C1 ; Cl]

1-(2-Isopropyl-2,3,4,5-tetrahydro-1H-benzofuro[3,2-c]azepin-8-yl)-4-((6-(trifluoromethyl)pyridin-2-yl)methoxy)pyridin-2(1H)-one (38 mg, 0.076 mmol) was suspended in MeOH (2 mL) and treated with 2 N HCl in Et₂O (38 μL, 0.076 mmol). The suspension was stirred at ambient temperature for 30 min. The solvent was removed, and the residue was co-evaporated with acetonitrile and lyophilized from acetonitrile-water to provide the title compound (41 mg, quant) as a white solid: ¹H NMR (500 MHz, DMSO-d₆) δ 9.97 (s, 1H), 8.21 (t, J=8.0 Hz, 1H), 7.94-7.83 (m, 3H), 7.66-7.63 (m, 2H), 7.30-7.28 (m, 1H), 6.21-6.19 (m, 1H), 6.00 (d, J=3.0 Hz, 1H), 5.33 (s, 2H), 4.64-4.53 (m, 2H), 3.68-3.51 (m, 3H), 3.12 (s, 2H), 2.26-2.12 (m, 2H), 1.38-1.33 (m, 6H); ESI MS m/z 498 [M+H]⁺; HPLC (Method B) 93.8% (AUC), t_{R}=14.6 min.

### Example 82

### Preparation of 1-(2-Methyl-2,3,4,5-tetrahydro-1H-benzofuro[3,2-c]azepin-8-yl)-4-(4-(trifluoromethyl)phenyl)pyridin-2(1H)-one hydrochloride

### a) 1-(2-Methyl-2,3,4,5-tetrahydro-1H-benzofuro[3,2-c]azepin-8-yl)-4-(4-(trifluoromethyl)phenyl)pyridin-2(1H)-one

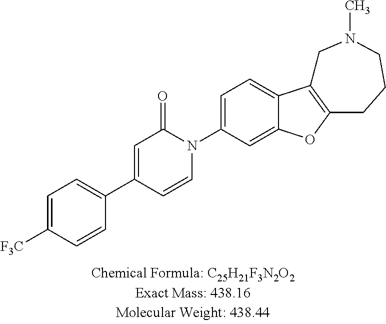 [SMILES: CN1CCCc2oc3cc(-n4ccc(-c5ccc(C(F)(F)F)cc5)cc4=O)ccc3c2C1]

A mixture of 1-(2,3,4,5-tetrahydro-1H-benzofuro[3,2-c]azepin-8-yl)-4-(4-(trifluoromethyl)phenyl)pyridin-2(1H)-one hydrochloride (65 mg, 0.14 mmol) in dichloromethane (0.8 mL) methanol (0.8 mL) was treated with 37% aqueous formaldehyde solution (29 μL, 0.35 mmol) followed by sodium triacetoxyborohydride (90 mg, 0.43 mmol). After stirring at ambient temperature for 1 h, the mixture was diluted with saturated aqueous NaHCO₃ solution (50 mL) and extracted with dichloromethane (5×50 mL). The combined organic extracts were dried over Na₂SO₄ and concentrated under reduced pressure. Purification by flash chromatography (silica gel, CH₂Cl₂/(90:9:1 CH₂Cl₂/MeOH/NH₄OH), 100:0 to 25:75) afforded the title compound (58 mg, 93%) as a white solid: ESI MS m/z 439 [M+H]⁺.

### b) 1-(2-Methyl-2,3,4,5-tetrahydro-1H-benzofuro[3,2-c]azepin-8-yl)-4-(4-(trifluoromethyl)phenyl)pyridin-2(1H)-one hydrochloride

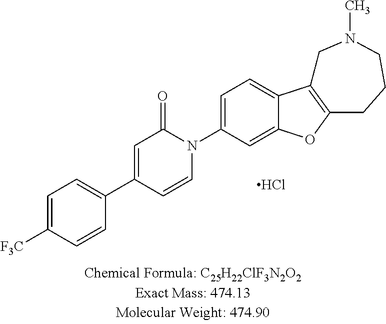 [SMILES: CN1CCCc2oc3cc(-n4ccc(-c5ccc(C(F)(F)F)cc5)cc4=O)ccc3c2C1 ; Cl]

1-(2-Methyl-2,3,4,5-tetrahydro-1H-benzofuro[3,2-c]azepin-8-yl)-4-(4-(trifluoromethyl)phenyl)pyridin-2(1H)-one (57 mg, 0.13 mmol) was suspended in MeOH (5 mL) and treated with 2 N HCl in Et₂O (65 μL, 0.13 mmol). The suspension was stirred at ambient temperature for 30 min. The solvent was removed, and the residue was co-evaporated with acetonitrile and lyophilized from acetonitrile-water to provide the title compound (59 mg, 96%) as a white solid: ¹H NMR (500 MHz, DMSO-d₆) δ 10.68 (s, 1H), 8.02-8.00 (m, 2H), 7.89-7.87 (m, 2H), 7.84-7.82 (m, 1H), 7.80-7.76 (m, 2H), 7.39-7.37 (m, 1H), 6.89 (d, J=2.0 Hz, 1H), 6.76-6.74 (m, 1H), 4.71-4.51 (m, 2H), 3.69-3.52 (m, 2H), 3.12 (t, J=6.0 Hz, 2H), 2.91 (s, 3H), 2.19-2.12 (m, 2H); ESI MS m/z 439 [M+H]⁺; HPLC (Method B)>99% (AUC), t_{R}=15.1 min.

### Example 83

### Preparation of 4-(2-(5-Fluoropyridin-2-yl)ethyl)-1-(2,3,4,5-tetrahydro-1H-benzofuro[3,2-c]azepin-8-yl)piperazin-2-one hydrochloride

### a) tert-Butyl 8-(4-(2-(5-fluoropyridin-2-yl)ethyl)-2-oxopiperazin-1-yl)-4,5-dihydro-1H-benzofuro[3,2-c]azepine-2(3H)-carboxylate

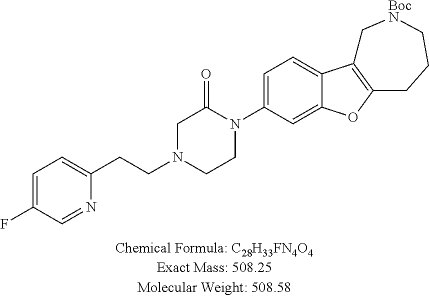 [SMILES: CC(C)(C)OC(=O)N1CCCc2oc3cc(N4CCN(CCc5ccc(F)cn5)CC4=O)ccc3c2C1]

A mixture of 4-(2-(5-fluoropyridin-2-yl)ethyl)piperazin-2-one (94 mg, 0.42 mmol), tert-butyl 8-bromo-4,5-dihydro-1H-benzofuro[3,2-c]azepine-2(3H)-carboxylate (128 mg, 0.349 mmol) and Cs₂CO₃ (148 mg, 0.454 mmol) in toluene (5 mL) in a sealed tube was degassed with a nitrogen stream for 10 min. Trans-N,N′-dimethylcyclohexane-1,2-diamine (75 mg, 0.53 mmol) and CuI (100 mg, 0.53 mmol) were added, and the mixture was degassed for another 2 min. The tube was sealed, and the mixture was heated at 110° C. for 12 h. The mixture was cooled, diluted with 90:9:1 CH₂Cl₂/MeOH/NH₄OH (15 mL) and stirred for 1 h. Then 2:1 brine/NH₄OH (100 mL) was added, and the aqueous phase was extracted with dichloromethane (4×75 mL). The combined organic extracts were washed with 2:1 brine/NH₄OH (3×75 mL), dried over Na₂SO₄, and concentrated under reduced pressure. Purification by flash chromatography (silica gel, CH₂Cl₂/(90:9:1 CH₂Cl₂/MeOH/NH₄OH), 100:0 to 25:75) afforded the title compound (109 mg, 61%) as a colorless solid: ESI MS m/z 509 [M+H]⁺.

### b) 4-(2-(5-Fluoropyridin-2-yl)ethyl)-1-(2,3,4,5-tetrahydro-1H-benzofuro[3,2-c]azepin-8-yl)piperazin-2-one

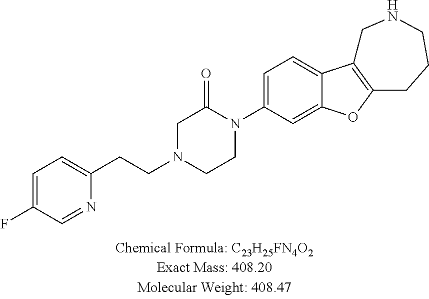 [SMILES: O=C1CN(CCc2ccc(F)cn2)CCN1c1ccc2c3c(oc2c1)CCCNC3]

A solution of tert-butyl 8-(4-(2-(5-fluoropyridin-2-yl)ethyl)-2-oxopiperazin-1-yl)-4,5-dihydro-1H-benzofuro[3,2-c]azepine-2(3H)-carboxylate (109 mg, 0.214 mmol) in MeOH (6 mL) was treated with 2 N HCl in Et₂O (4 mL), and the resulting solution was stirred at ambient temperature for 12 h. Saturated aqueous NaHCO₃ (40 mL) was added, and the mixture was extracted with dichloromethane (8×40 mL). The combined organic extracts were dried over Na₂SO₄ and concentrated under reduced pressure. Purification by flash chromatography (silica gel, CH₂Cl₂/(90:9:1 CH₂Cl₂/MeOH/NH₄OH), 100:0 to 10:90) afforded the title compound (81 mg, 93%) as a white solid: ESI MS m/z 409 [M+H]⁺.

### c) 4-(2-(5-Fluoropyridin-2-yl)ethyl)-1-(2,3,4,5-tetrahydro-1H-benzofuro[3,2-c]azepin-8-yl)piperazin-2-one hydrochloride

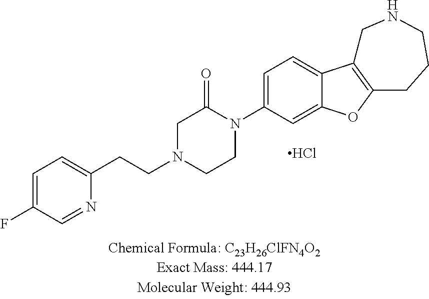 [SMILES: O=C1CN(CCc2ccc(F)cn2)CCN1c1ccc2c3c(oc2c1)CCCNC3 ; Cl]

4-(2-(5-Fluoropyridin-2-yl)ethyl)-1-(2,3,4,5-tetrahydro-1H-benzofuro[3,2-c]azepin-8-yl)piperazin-2-one (81 mg, 0.20 mmol) was suspended in MeOH (6 mL) and treated with 2 N HCl in Et₂O (99 μL, 0.20 mmol). The suspension was stirred at ambient temperature for 30 min. The solvent was removed, and the residue was co-evaporated with acetonitrile and lyophilized from acetonitrile-water to provide the title compound (86 mg, 97%) as a white solid: ¹H NMR (500 MHz, DMSO-d₆) δ 9.21 (s, 2H), 8.49 (s, 1H), 7.67 (d, J=8.0 Hz, 2H), 7.55 (d, J=1.5 Hz, 1H), 7.45-7.42 (m, 1H), 7.25-7.23 (m, 1H), 4.37 (s, 2H), 3.68-3.46 (m, 4H), 3.27-2.65 (m, 10H), 2.09-2.05 (m, 2H); ESI MS m/z 409 [M+H]⁺; HPLC (Method B)>99% (AUC), t_{R}=10.0 min.

### Example 84

### Preparation of 1-(3-Methyl-2,3,4,5-tetrahydro-1H-benzofuro[2,3-d]azepin-8-yl)-4-((6-(trifluoromethyl)pyridin-3-yl)methoxy)pyridin-2(1H)-one hydrochloride

### a) 1-(3-Methyl-2,3,4,5-tetrahydro-1H-benzofuro[2,3-d]azepin-8-yl)-4-((6-(trifluoromethyl)pyridin-3-yl)methoxy)pyridin-2(1H)-one

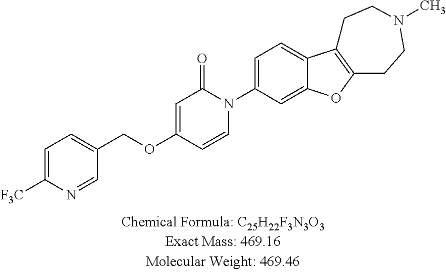 [SMILES: CN1CCc2oc3cc(-n4ccc(OCc5ccc(C(F)(F)F)nc5)cc4=O)ccc3c2CC1]

A mixture of 1-(2,3,4,5-tetrahydro-1H-benzofuro[2,3-d]azepin-8-yl)-4-((6-(trifluoromethyl)pyridin-3-yl)methoxy)pyridin-2(1H)-one hydrochloride (46 mg, 0.094 mmol) in dichloromethane (0.6 mL) and methanol (0.6 mL) was treated with 37% aqueous formaldehyde solution (20 μL, 0.23 mmol) followed by sodium triacetoxyborohydride (59 mg, 0.28 mmol). After stirring at ambient temperature for 1 h, the mixture was diluted with saturated aqueous NaHCO₃ solution (50 mL) and extracted with dichloromethane (5×50 mL). The combined organic extracts were dried over Na₂SO₄ and concentrated under reduced pressure. Purification by flash chromatography (silica gel, CH₂Cl₂/(90:9:1 CH₂Cl₂/MeOH/NH₄OH), 100:0 to 25:75) afforded the title compound (40 mg, 91%) as a white solid: ESI MS m/z 470 [M+H]⁺.

### b) 1-(3-Methyl-2,3,4,5-tetrahydro-1H-benzofuro[2,3-d]azepin-8-yl)-4-((6-(trifluoromethyl)pyridin-3-yl)methoxy)pyridin-2(1H)-one hydrochloride

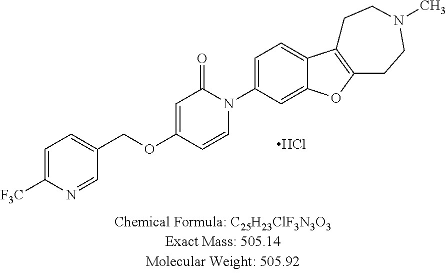 [SMILES: CN1CCc2oc3cc(-n4ccc(OCc5ccc(C(F)(F)F)nc5)cc4=O)ccc3c2CC1 ; Cl]

1-(3-Methyl-2,3,4,5-tetrahydro-1H-benzofuro[2,3-d]azepin-8-yl)-4-((6-(trifluoromethyl)pyridin-3-yl)methoxy)pyridin-2(1H)-one (38 mg, 0.081 mmol) was suspended in MeOH (5 mL) and treated with 2 N HCl in Et₂O (41 μL, 0.082 mmol). The suspension was stirred at ambient temperature for 30 min. The solvent was removed, and the residue was co-evaporated with acetonitrile and lyophilized from acetonitrile-water to provide the title compound (41 mg, quant) as a white solid: ¹H NMR (500 MHz, DMSO-d₆) δ 10.97 (s, 1H), 8.88 (s, 1H), 8.20-8.18 (m, 1H), 7.99 (d, J=8.0 Hz, 1H), 7.66-7.59 (m, 3H), 7.24-7.22 (m, 1H), 6.17-6.15 (m, 1H), 6.03 (d, J=3.0 Hz, 1H), 5.35 (s, 2H), 3.65-3.05 (m, 8H), 2.93 (s, 3H); ESI MS m/z 470 [M+H]⁺; HPLC (Method B)>99% (AUC), t_{R}=14.0 min.

### Example 85

### Preparation of 1-(2-Methyl-2,3,4,5-tetrahydro-1H-benzofuro[3,2-c]azepin-8-yl)-4-((6-methylpyridin-3-yl)methoxy)pyridin-2(1H)-one hydrochloride

### a) 1-(2-Methyl-2,3,4,5-tetrahydro-1H-benzofuro[3,2-c]azepin-8-yl)-4-((6-methylpyridin-3-yl)methoxy)pyridin-2(1H)-one

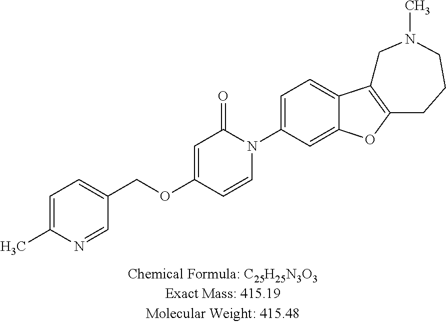 [SMILES: Cc1ccc(COc2ccn(-c3ccc4c5c(oc4c3)CCCN(C)C5)c(=O)c2)cn1]

A mixture of 4-((6-methylpyridin-3-yl)methoxy)-1-(2,3,4,5-tetrahydro-1H-benzofuro[3,2-c]azepin-8-yl)pyridin-2(1H)-one hydrochloride (56 mg, 0.13 mmol) in dichloromethane (0.8 mL) and methanol (0.8 mL) was treated with 37% aqueous formaldehyde solution (26 μL, 0.32 mmol) followed by sodium triacetoxyborohydride (81 mg, 0.38 mmol). After stirring at ambient temperature for 1 h, the mixture was diluted with saturated aqueous NaHCO₃ solution (50 mL) and extracted with dichloromethane (5×50 mL). The combined organic extracts were dried over Na₂SO₄ and concentrated under reduced pressure. Purification by flash chromatography (silica gel, CH₂Cl₂/(90:9:1 CH₂Cl₂/MeOH/NH₄OH), 100:0 to 25:75) afforded the title compound (45 mg, 85%) as a colorless solid: ESI MS m/z 416 [M+H]⁺.

### b) 1-(2-Methyl-2,3,4,5-tetrahydro-1H-benzofuro[3,2-c]azepin-8-yl)-4-((6-methylpyridin-3-yl)methoxy)pyridin-2(1H)-one hydrochloride

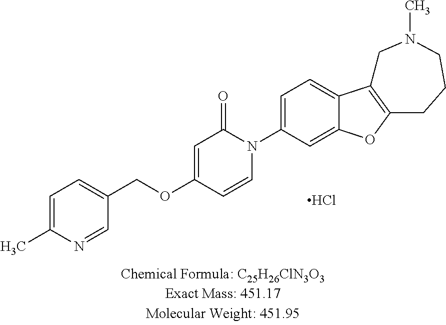 [SMILES: Cc1ccc(COc2ccn(-c3ccc4c5c(oc4c3)CCCN(C)C5)c(=O)c2)cn1 ; Cl]

1-(2-Methyl-2,3,4,5-tetrahydro-1H-benzofuro[3,2-c]azepin-8-yl)-4-((6-methylpyridin-3-yl)methoxy)pyridin-2(1H)-one (45 mg, 0.11 mmol) was suspended in MeOH (2 mL) and treated with 2 N HCl in Et₂O (54 μL, 0.11 mmol). The suspension was stirred at ambient temperature for 30 min. The solvent was removed, and the residue was co-evaporated with acetonitrile and lyophilized from acetonitrile-water to provide the title compound (49 mg, quant) as a white solid: ¹H NMR (500 MHz, DMSO-d₆) δ 10.60 (s, 1H), 8.59 (s, 1H), 7.87-7.86 (m, 1H), 7.73 (d, J=8.5 Hz, 1H), 7.64-7.59 (m, 2H), 7.39 (d, J=8.0 Hz, 1H), 7.28-7.26 (m, 1H), 6.13-6.11 (m, 1H), 6.02 (d, J=2.5 Hz, 1H), 5.18 (s, 2H), 4.72-4.70 (m, 1H), 4.48-4.46 (m, 1H), 3.72-3.68 (m, 1H), 3.51-3.50 (m, 1H), 3.10 (t, J=6.0 Hz, 2H), 2.90 (s, 3H), 2.52-2.50 (m, 3H), 2.19-2.09 (m, 2H); ESI MS m/z 416 [M+H]⁺; HPLC (Method B)>99% (AUC), t_{R}=10.1 min.

### Example 86

### Preparation of 1-(2-Methyl-2,3,4,5-tetrahydro-1H-benzofuro[3,2-c]azepin-8-yl)-4-((6-(trifluoromethyl)pyridin-3-yl)methoxy)pyridin-2(1H)-one hydrochloride

### a) 1-(2-Methyl-2,3,4,5-tetrahydro-1H-benzofuro[3,2-c]azepin-8-yl)-4-((6-(trifluoromethyl)pyridin-3-yl)methoxy)pyridin-2(1H)-one

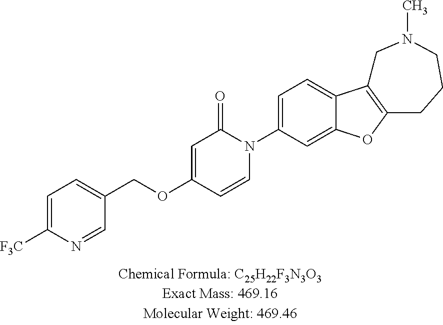 [SMILES: CN1CCCc2oc3cc(-n4ccc(OCc5ccc(C(F)(F)F)nc5)cc4=O)ccc3c2C1]

A mixture of 1-(2,3,4,5-tetrahydro-1H-benzofuro[3,2-c]azepin-8-yl)-4-((6-(trifluoromethyl)pyridin-3-yl)methoxy)pyridin-2(1H)-one hydrochloride (70 mg, 0.14 mmol) in dichloromethane (0.9 mL) and methanol (0.9 mL) was treated with 37% aqueous formaldehyde solution (28 μL, 0.36 mmol) followed by sodium triacetoxyborohydride (90 mg, 0.43 mmol). After stirring at ambient temperature for 1 h, the mixture was diluted with saturated aqueous NaHCO₃ solution (50 mL) and extracted with dichloromethane (5×50 mL). The combined organic extracts were dried over Na₂SO₄ and concentrated under reduced pressure. Purification by flash chromatography (silica gel, CH₂Cl₂/(90:9:1 CH₂Cl₂/MeOH/NH₄OH), 100:0 to 25:75) afforded the title compound (63 mg, 94%) as a white solid: ESI MS m/z 470 [M+H]⁺.

### b) 1-(2-Methyl-2,3,4,5-tetrahydro-1H-benzofuro[3,2-c]azepin-8-yl)-4-((6-(trifluoromethyl)pyridin-3-yl)methoxy)pyridin-2(1H)-one hydrochloride

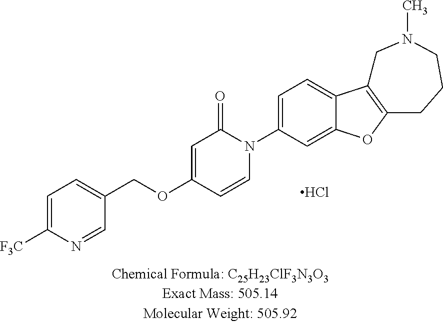 [SMILES: CN1CCCc2oc3cc(-n4ccc(OCc5ccc(C(F)(F)F)nc5)cc4=O)ccc3c2C1 ; Cl]

1-(2-Methyl-2,3,4,5-tetrahydro-1H-benzofuro[3,2-c]azepin-8-yl)-4-((6-(trifluoromethyl)pyridin-3-yl)methoxy)pyridin-2(1H)-one (63 mg, 0.13 mmol) was suspended in MeOH (2.5 mL) and treated with 2 N HCl in Et₂O (67 μL. 0.13 mmol). The suspension was stirred at ambient temperature for 30 min. The solvent was removed, and the residue was co-evaporated with acetonitrile and lyophilized from acetonitrile-water to provide the title compound (68 mg, quant) as a white solid: ¹H NMR (500 MHz, DMSO-d₆) δ 10.60 (s, 1H), 8.89 (s, 1H), 8.20-8.18 (m, 1H), 7.99 (d, J=8.0 Hz, 1H), 7.74 (d, J=8.0 Hz, 1H), 7.65-7.63 (m, 2H), 7.29-7.27 (m, 1H), 6.18-6.16 (m, 1H), 6.03 (d, J=3.0 Hz, 1H), 5.35 (s, 2H), 4.72-4.46 (m, 2H), 3.69-3.50 (m, 2H), 3.10 (t, J=6.0 Hz, 2H), 2.90 (s, 3H), 2.18-2.07 (m, 2H); ESI MS m/z 470 [M+H]⁺; HPLC (Method B)>99% (AUC), t_{R}=14.0 min.

### Example 87

### Preparation of 4-(4-Chlorophenethyl)-1-(1,2,3,4-tetrahydrobenzofuro[3,2-c]pyridin-7-yl)piperazin-2-one hydrochloride

### a) 4-(4-Chlorophenethyl)piperazin-2-one

CAS Registry Number 1260401-03-3

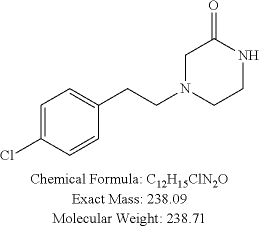 [SMILES: O=C1CN(CCc2ccc(Cl)cc2)CCN1]

This compound was prepared in accordance with the procedure described in PCT Publication No. WO 2011/003005 to Guzzo et al., which is hereby incorporated by reference in its entirety.

### b) tert-Butyl 7-(4-(4-chlorophenethyl)-2-oxopiperazin-1-yl)-3,4-dihydrobenzofuro[3,2-c]pyridine-2(1H)-carboxylate

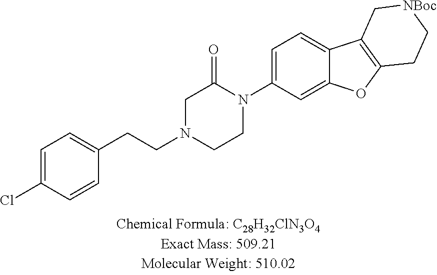 [SMILES: CC(C)(C)OC(=O)N1CCc2oc3cc(N4CCN(CCc5ccc(Cl)cc5)CC4=O)ccc3c2C1]

tert-Butyl 7-bromo-3,4-dihydrobenzofuro[3,2-c]pyridine-2(1H)-carboxylate (279 mg, 0.792 mmol) and 4-(4-chlorophenethyl)piperazin-2-one (189 mg, 0.792 mmol) were reacted according to Example 3 (step b) to provide the title compound (217 mg, 54%) as a white solid: ¹H NMR (500 MHz, CDCl₃) δ 7.42 (d, J=8.0 Hz, 1H), 7.38 (d, J=1.5 Hz, 1H), 7.28 (d, J=8.5 Hz, 2H), 7.16 (d, J=8.5 Hz, 2H), 7.14 (s, 1H), 4.54 (s, 2H), 3.82 (s, 2H), 3.73 (t, J=5.5 Hz, 2H), 3.41 (s, 2H), 2.89 (t, J=5.5 Hz, 2H), 2.85-2.82 (m, 4H), 2.74-2.70 (m, 2H), 1.50 (s, 9H); ESI MS m/z 510 [M+H]⁺.

### c) 4-(4-Chlorophenethyl)-1-(1,2,3,4-tetrahydrobenzofuro[3,2-c]pyridin-7-yl)piperazin-2-one

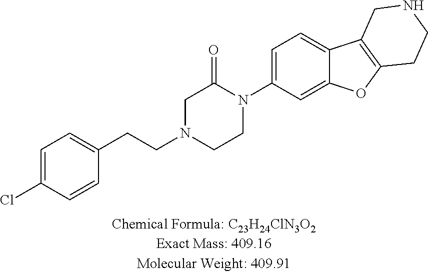 [SMILES: O=C1CN(CCc2ccc(Cl)cc2)CCN1c1ccc2c3c(oc2c1)CCNC3]

A solution of tert-butyl 7-(4-(4-chlorophenethyl)-2-oxopiperazin-1-yl)-3,4-dihydrobenzofuro[3,2-c]pyridine-2(1H)-carboxylate (216 mg, 0.424 mmol) in MeOH (5.0 mL) was treated with 2 N HCl in Et₂O (10 mL), and the resulting solution was stirred at ambient temperature for 16 h and then concentrated in vacuo. The resultant HCl salt was converted to the corresponding free base using an SCX-2 cartridge to provide the title compound (177 mg, quant. yield) as a white solid: ¹H NMR (500 MHz, CDCl₃) δ 7.40-7.39 (m, 2H), 7.29-7.27 (m, 2H), 7.17-7.15 (m, 3H), 4.15 (s, 2H), 3.73 (t, J=5.5 Hz, 2H), 3.41-3.38 (m, 4H), 2.99 (t, J=5.5 Hz, 2H), 2.89 (t, J=5.5 Hz, 2H), 2.85-2.81 (m, 2H), 2.74-2.70 (m, 2H); ESI MS m/z 410 [M+H]⁺.

### d) 4-(4-Chlorophenethyl)-1-(1,2,3,4-tetrahydrobenzofuro[3,2-c]pyridin-7-yl)piperazin-2-one hydrochloride

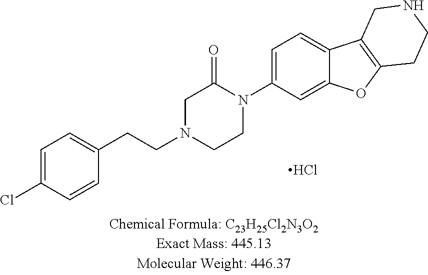 [SMILES: O=C1CN(CCc2ccc(Cl)cc2)CCN1c1ccc2c3c(oc2c1)CCNC3 ; Cl]

A solution of 4-(4-chlorophenethyl)-1-(1,2,3,4-tetrahydrobenzofuro[3,2-c]pyridin-7-yl)piperazin-2-one (176 mg, 0.429 mmol) in MeOH (5.0 mL) was treated with 2 N HCl in Et₂O (0.22 mL, 0.43 mmol) and the resulting suspension was concentrated. The solid was dissolved in H₂O (3 mL), frozen, and lyophilized overnight to provide the title compound (197 mg, quant. yield) as a white solid: ¹H NMR (500 MHz, CD₃OD) δ 7.64 (d, J=8.0 Hz, 1H), 7.60 (d, J=1.5 Hz, 1H), 7.39-7.35 (m, 4H), 7.32 (dd, J=8.3, 1.8 Hz, 1H), 4.45 (s, 2H), 4.21 (s, 2H), 4.10 (br s, 2H), 3.85 (br s, 2H), 3.69 (t, J=6.3 Hz, 2H), 3.58 (t, J=8.3 Hz, 2H), 3.21-3.16 (m, 4H); ESI MS m/z 410 [M+H]⁺; HPLC (Method A)>99% (AUC), t_{R}=11.8 min.

### Example 88

### Preparation of 1-(1,2,3,4-Tetrahydrobenzofuro[3,2-c]pyridin-7-yl)-4-(6-(trifluoromethyl)pyridazin-3-yl)pyridin-2(1H)-one hydrochloride

### a) 4-(6-(Trifluoromethyl)pyridazin-3-yl)pyridin-2(1H)-one

CAS Registry Number 1173155-66-2

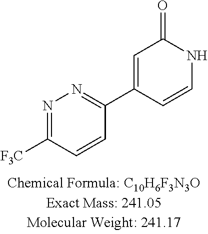 [SMILES: O=c1cc(-c2ccc(C(F)(F)F)nn2)cc[nH]1]

This compound was prepared in accordance with the procedure described in PCT Publication No. WO 2009/089482 to Guzzo et al., which is hereby incorporated by reference in its entirety.

### b) tert-Butyl 7-(2-oxo-4-(6-(trifluoromethyl)pyridazin-3-yl)pyridin-1(2H)-yl)-3,4-dihydrobenzofuro[3,2-c]pyridine-2(1H)-carboxylate

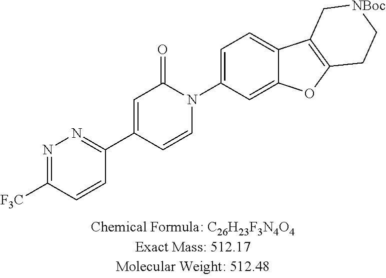 [SMILES: CC(C)(C)OC(=O)N1CCc2oc3cc(-n4ccc(-c5ccc(C(F)(F)F)nn5)cc4=O)ccc3c2C1]

tert-Butyl 7-bromo-3,4-dihydrobenzofuro[3,2-c]pyridine-2(1H)-carboxylate (242 mg, 0.687 mmol) and 4-(6-(trifluoromethyl)pyridazin-3-yl)pyridin-2(1H)-one (166 mg, 0.688 mmol) were reacted according to Example 3 (step b) to provide the title compound (294 mg, 83%) as a yellow solid: ¹H NMR (500 MHz, CDCl₃) δ 8.10 (d, J=9.0 Hz, 1H), 7.98 (d, J=9.0 Hz, 1H), 7.62 (d, J=7.0 Hz, 1H), 7.56 (d, J=2.0 Hz, 1H), 7.54 (s, 1H), 7.29 (dd, J=7.3, 1.8 Hz, 2H), 7.25 (d, J=2.0 Hz, 1H), 4.59 (s, 2H), 3.86 (s, 2H), 2.90 (s, 2H), 1.52 (s, 9H); ESI MS m/z 513 [M+H]⁺.

### c) 1-(1,2,3,4-Tetrahydrobenzofuro[3,2-c]pyridin-7-yl)-4-(6-(trifluoromethyl)pyridazin-3-yl)pyridin-2(1H)-one

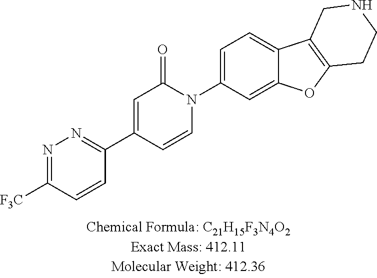 [SMILES: O=c1cc(-c2ccc(C(F)(F)F)nn2)ccn1-c1ccc2c3c(oc2c1)CCNC3]

A solution of tert-butyl 7-(2-oxo-4-(6-(trifluoromethyl)pyridazin-3-yl)pyridin-1(2H)-yl)-3,4-dihydrobenzofuro[3,2-c]pyridine-2(1H)-carboxylate (293 mg, 0.572 mmol) in MeOH (5.0 mL) was treated with 2 N HCl in Et₂O (10 mL), and the resulting solution was stirred at ambient temperature for 16 h and then concentrated in vacuo. The resultant HCl salt was converted to the corresponding free base using an SCX-2 cartridge to provide the title compound (199 mg, 85%) as a yellow solid: ¹H NMR (500 MHz, CDCl₃) δ 8.10 (d, J=9.0 Hz, 1H), 7.98 (d, J=9.0 Hz, 1H), 7.63 (d, J=7.5 Hz, 1H), 7.54 (d, J=1.5 Hz, 1H), 7.50 (d, J=8.5 Hz, 1H), 7.29-7.24 (m, 3H), 4.02 (s, 2H), 3.27 (t, J=5.8 Hz, 2H), 2.84 (t, J=5.5 Hz, 2H); ESI MS m/z 413 [M+H]⁺.

### d) 1-(1,2,3,4-Tetrahydrobenzofuro[3,2-c]pyridin-7-yl)-4-(6-(trifluoromethyl)pyridazin-3-yl)pyridin-2(1H)-one hydrochloride

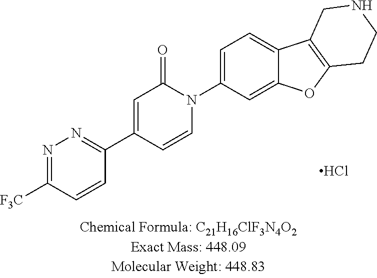 [SMILES: O=c1cc(-c2ccc(C(F)(F)F)nn2)ccn1-c1ccc2c3c(oc2c1)CCNC3 ; Cl]

A solution of 1-(1,2,3,4-tetrahydrobenzofuro[3,2-c]pyridin-7-yl)-4-(6-(trifluoromethyl)pyridazin-3-yl)pyridin-2(1H)-one (193 mg, 0.468 mmol) in MeOH (5.0 mL) was treated with 2 N HCl in Et₂O (0.24 mL, 0.47 mmol), and the resulting suspension was concentrated. The solid was suspended in H₂O (3 mL), frozen, and lyophilized overnight to provide the title compound (199 mg, 94%) as a yellow solid: mp>300° C.; ¹H NMR (500 MHz, CD₃OD) δ 8.53 (d, J=9.0 Hz, 1H), 8.27 (d, J=9.0 Hz, 1H), 7.89 (d, J=7.5 Hz, 1H), 7.74-7.72 (m, 2H), 7.44 (d, J=1.5 Hz, 1H), 7.41 (dd, J=8.3, 1.8 Hz, 1H), 7.34 (dd, J=7.3, 1.8 Hz, 1H), 4.49 (s, 2H), 3.71 (t, J=6.0 Hz, 2H), 3.23 (t, J=6.0 Hz, 2H); ESI MS m/z 413 [M+H]⁺; HPLC (Method A)>99% (AUC), t_{R}=13.0 min.

### Example 89

### Preparation of 4-(4-Chlorophenethyl)-1-(2,3,4,5-tetrahydro-1H-benzofuro[3,2-c]azepin-8-yl)piperazin-2-one hydrochloride

### a) tert-Butyl 8-(4-(4-chlorophenethyl)-2-oxopiperazin-1-yl)-4,5-dihydro-1H-benzofuro[3,2-c]azepine-2(3H)-carboxylate

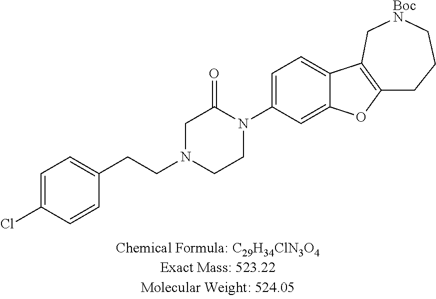 [SMILES: CC(C)(C)OC(=O)N1CCCc2oc3cc(N4CCN(CCc5ccc(Cl)cc5)CC4=O)ccc3c2C1]

A mixture of 4-(4-chlorophenethyl)piperazin-2-one (150 mg, 0.63 mmol), tert-butyl 8-bromo-4,5-dihydro-1H-benzofuro[3,2-c]azepine-2(3H)-carboxylate (192 mg, 0.524 mmol) and Cs₂CO₃ (225 mg, 0.691 mmol) in toluene (8 mL) in a sealed tube was degassed with a nitrogen stream for 10 min. Trans-N,N′-dimethylcyclohexane-1,2-diamine (112 mg, 0.787 mmol) and CuI (150 mg, 0.79 mmol) were added, and the mixture was degassed for another 2 min. The tube was sealed, and the mixture was heated at 110° C. for 12 h. The mixture was cooled, diluted with 90:9:1 CH₂Cl₂/MeOH/NH₄OH (15 mL) and stirred for 1 h. Then 2:1 brine/NH₄OH (100 mL) added, and the aqueous phase was extracted with dichloromethane (4×75 mL). The combined organic extracts were washed with 2:1 brine/NH₄OH (3×75 mL), dried over Na₂SO₄ and concentrated under reduced pressure. Purification by flash chromatography (silica gel, CH₂Cl₂/(90:9:1 CH₂Cl₂/MeOH/NH₄OH), 100:0 to 25:75) afforded the title compound (178 mg, 65%) as a colorless solid: ESI MS m/z 524 [M+H]⁺.

### b) 4-(4-Chlorophenethyl)-1-(2,3,4,5-tetrahydro-1H-benzofuro[3,2-c]azepin-8-yl)piperazin-2-one

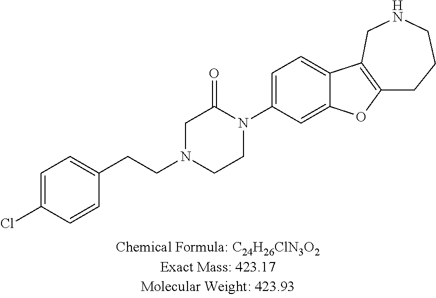 [SMILES: O=C1CN(CCc2ccc(Cl)cc2)CCN1c1ccc2c3c(oc2c1)CCCNC3]

A solution of tert-butyl 8-(4-(4-chlorophenethyl)-2-oxopiperazin-1-yl)-4,5-dihydro-1H-benzofuro[3,2-c]azepine-2(3H)-carboxylate (178 mg, 0.340 mmol) in MeOH (8 mL) was treated with 2 N HCl in Et₂O (4 mL, 8 mmol), and the resulting solution was stirred at ambient temperature for 12 h. Saturated aqueous NaHCO₃ (40 mL) was added, and the mixture was extracted with dichloromethane (8×40 mL). The combined organic extracts were dried over Na₂SO₄ and concentrated under reduced pressure. Purification by flash chromatography (silica gel, CH₂Cl₂/(90:9:1 CH₂Cl₂/MeOH/NH₄OH), 100:0 to 10:90) afforded the title compound (118 mg, 82%) as a white solid: ESI MS m/z 424 [M+H]⁺.

### c) 4-(4-Chlorophenethyl)-1-(2,3,4,5-tetrahydro-1H-benzofuro[3,2-c]azepin-8-yl)piperazin-2-one hydrochloride

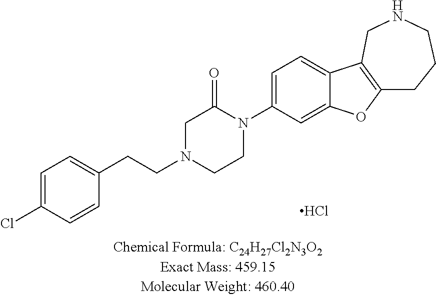 [SMILES: O=C1CN(CCc2ccc(Cl)cc2)CCN1c1ccc2c3c(oc2c1)CCCNC3 ; Cl]

4-(4-Chlorophenethyl)-1-(2,3,4,5-tetrahydro-1H-benzofuro[3,2-c]azepin-8-yl)piperazin-2-one (114 mg, 0.269 mmol) was suspended in MeOH (10 mL) and treated with 2 N HCl in Et₂O (134 μL, 0.268 mmol). The suspension was stirred at ambient temperature for 30 min. The solvent was removed, and the residue was co-evaporated with acetonitrile and lyophilized from acetonitrile-water to provide the title compound (126 mg, quant) as a white solid: ¹H NMR (500 MHz, DMSO-d₆) δ 9.33 (s, 2H), 7.66 (d, J=8.5 Hz, 1H), 7.55 (d, J=1.5 Hz, 1H), 7.36-7.30 (m, 4H), 7.24 (dd, J=8.5, 1.5 Hz, 1H), 4.36 (s, 2H), 3.73-3.65 (m, 2H), 3.46-3.44 (m, 2H), 3.25 (s, 2H), 3.08 (t, J=6.0 Hz, 2H), 2.86-2.79 (m, 4H), 2.71-2.66 (m, 2H), 2.17-2.12 (m, 2H); ESI MS m/z 424 [M+H]⁺; HPLC (Method B)>99% (AUC), t_{R}=11.9 min.

### Example 90

### Preparation of 4-(2-(5-Chloropyridin-2-yl)ethyl)-1-(2,3,4,5-tetrahydro-1H-benzofuro[3,2-c]azepin-8-yl)piperazin-2-one hydrochloride

### a) tert-Butyl 8-(4-(2-(5-chloropyridin-2-yl)ethyl)-2-oxopiperazin-1-yl)-4,5-dihydro-1H-benzofuro[3,2-c]azepine-2(3H)-carboxylate

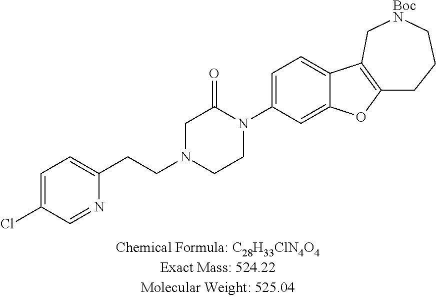 [SMILES: CC(C)(C)OC(=O)N1CCCc2oc3cc(N4CCN(CCc5ccc(Cl)cn5)CC4=O)ccc3c2C1]

A mixture of 4-(2-(5-chloropyridin-2-yl)ethyl)piperazin-2-one (151 mg, 0.626 mmol), tert-butyl 8-bromo-4,5-dihydro-1H-benzofuro[3,2-c]azepine-2(3H)-carboxylate (192 mg, 0.524 mmol) and Cs₂CO₃ (225 mg, 0.691 mmol) in toluene (8 mL) in a sealed tube was degassed with a nitrogen stream for 10 min. Trans-N,N′-dimethylcyclohexane-1,2-diamine (112 mg, 0.787 mmol) and CuI (150 mg, 0.79 mmol) were added, and the mixture was degassed for another 2 min. The tube was sealed, and the mixture was heated at 110° C. for 12 h. The mixture was cooled, diluted with 90:9:1 CH₂Cl₂/MeOH/NH₄OH (15 mL) and stirred for 1 h. Then 2:1 brine/NH₄OH (100 mL) was added, and the aqueous phase was extracted with dichloromethane (4×75 mL). The combined organic extracts were washed with 2:1 brine/NH₄OH (3×75 mL), dried over Na₂SO₄ and concentrated under reduced pressure. Purification by flash chromatography (silica gel, CH₂Cl₂/(90:9:1 CH₂Cl₂/MeOH/NH₄OH), 100:0 to 25:75) afforded the title compound (172 mg, 62%) as a colorless solid: ESI MS m/z 525 [M+H]⁺.

### b) 4-(2-(5-Chloropyridin-2-yl)ethyl)-1-(2,3,4,5-tetrahydro-1H-benzofuro[3,2-c]azepin-8-yl)piperazin-2-one

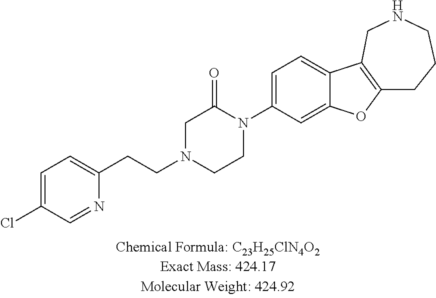 [SMILES: O=C1CN(CCc2ccc(Cl)cn2)CCN1c1ccc2c3c(oc2c1)CCCNC3]

A solution of tert-butyl 8-(4-(2-(5-chloropyridin-2-yl)ethyl)-2-oxopiperazin-1-yl)-4,5-dihydro-1H-benzofuro[3,2-c]azepine-2(3H)-carboxylate (172 mg, 0.328 mmol) in MeOH (8 mL) was treated with 2 N HCl in Et₂O (4 mL, 8 mmol), and the resulting solution was stirred at ambient temperature for 12 h. Saturated aqueous NaHCO₃ (40 mL) was added, and the mixture was extracted with dichloromethane (8×40 mL). The combined organic extracts were dried over Na₂SO₄ and concentrated under reduced pressure. Purification by flash chromatography (silica gel, CH₂Cl₂/(90:9:1 CH₂Cl₂/MeOH/NH₄OH), 100:0 to 25:75) afforded the title compound (127 mg, 91%) as a white solid: ESI MS m/z 425 [M+H]⁺.

### c) 4-(2-(5-Chloropyridin-2-yl)ethyl)-1-(2,3,4,5-tetrahydro-1H-benzofuro[3,2-c]azepin-8-yl)piperazin-2-one hydrochloride

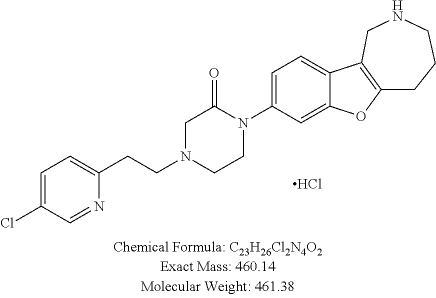 [SMILES: O=C1CN(CCc2ccc(Cl)cn2)CCN1c1ccc2c3c(oc2c1)CCCNC3 ; Cl]

4-(2-(5-Chloropyridin-2-yl)ethyl)-1-(2,3,4,5-tetrahydro-1H-benzofuro[3,2-c]azepin-8-yl)piperazin-2-one (122 mg, 0.287 mmol) was suspended in MeOH (10 mL) and treated with 2 N HCl in Et₂O (143 μL, 0.286 mmol). The suspension was stirred at ambient temperature for 30 min. The solvent was removed, and the residue was co-evaporated with acetonitrile and lyophilized from acetonitrile-water to provide the title compound (133 mg, quant) as a white solid: ¹H NMR (500 MHz, DMSO-d₆) δ 9.29 (s, 2H), 8.55-8.54 (m, 1H), 7.87-7.85 (m, 1H), 7.66 (d, J=8.0 Hz, 1H), 7.54 (d, J=2.0 Hz, 1H), 7.42 (d, J=8.0 Hz, 1H), 7.25-7.23 (m, 1H), 4.37 (s, 2H), 3.75-3.40 (m, 4H), 3.25-2.75 (m, 10H), 2.08-2.06 (m, 2H); ESI MS m/z 425 [M+H]⁺; HPLC (Method B)>99% (AUC), t_{R}=10.7 min.

### Example 91

### Preparation of 4-(2-(5-Chloropyridin-2-yl)ethyl)-1-(1,2,3,4-tetrahydrobenzofuro[3,2-c]pyridin-7-yl)piperazin-2-one hydrochloride

### a) 2-(5-Chloropyridin-2-yl)ethanol

CAS Registry Number 711017-56-0

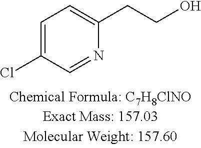

This compound was prepared in accordance with the procedure described in Publication No. US 2010/0331341 to Andersson et al., which is hereby incorporated by reference in its entirety.

### b) 2-(2-Chloroethyl)-5-chloropyridine

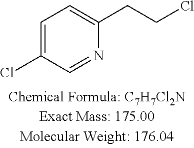

A solution of 2-(5-chloropyridin-2-yl)ethanol (3.94 g, 25.0 mmol) in anhydrous tetrahydrofuran (13 mL) was treated with thionyl chloride (2.7 mL, 37 mmol) slowly. The reaction was heated to reflux and stirred for 16 h. After cooling to rt, the reaction mixture was concentrated, and the residue was partitioned between ethyl acetate and saturated sodium bicarbonate. The aqueous phase was extracted with additional ethyl acetate (2×) and the combined organics were washed with brine, dried over sodium sulfate, filtered, and concentrated. Flash chromatography (120 g ISCO Gold column, 2%-20% ethyl acetate/hexanes) provided the title compound (1.60 g, 36%) as a yellow oil: ¹H NMR (500 MHz, CDCl₃) δ 8.52 (d, J=2.5 Hz, 1H), 7.61 (dd, J=8.5, 2.5 Hz, 1H), 7.17 (d, J=8.0 Hz, 1H), 3.91 (t, J=6.5 Hz, 2H), 3.21 (t, J=6.5 Hz, 2H); ESI MS m/z 176 [M+H]⁺.

### c) 4-(2-(5-Chloropyridin-2-yl)ethyl)piperazin-2-one

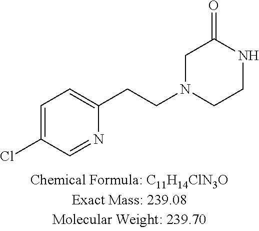 [SMILES: O=C1CN(CCc2ccc(Cl)cn2)CCN1]

A mixture of 2-(2-chloroethyl)-5-chloropyridine (1.60 g, 9.09 mmol) and piperazinone (910 mg, 9.09 mmol) in diisopropylethylamine (3.2 mL) was heated to reflux and stirred for 16 h. The reaction mixture was concentrated and purified by flash chromatography (120 g ISCO Gold column; 12%-100% B method, A=CH₂Cl₂, B=80:18:2 CH₂Cl₂/MeOH/NH₄OH) to provide the title compound (1.68 g, 77%) as a light yellow solid: ¹H NMR (500 MHz, CDCl₃) δ 8.48 (d, J=2.5 Hz, 1H), 7.58 (dd, J=8.0, 2.5 Hz, 1H), 7.14 (d, J=8.5 Hz, 1H), 5.99 (br s, 1H), 3.37-3.34 (m, 2H), 3.21 (s, 2H), 2.97 (t, J=7.5 Hz, 2H), 2.86-2.83 (m, 2H), 2.72 (t, J=5.5 Hz, 2H); ESI MS m/z 240 [M+H]⁺.

### d) tert-Butyl 7-(4-(2-(5-chloropyridin-2-yl)ethyl)-2-oxopiperazin-1-yl)-3,4-dihydrobenzofuro[3,2-c]pyridine-2(1H)-carboxylate

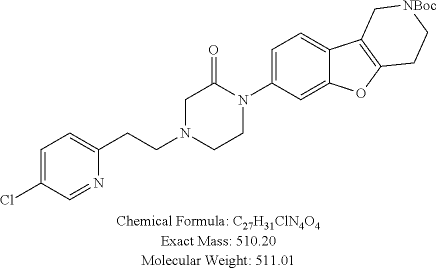 [SMILES: CC(C)(C)OC(=O)N1CCc2oc3cc(N4CCN(CCc5ccc(Cl)cn5)CC4=O)ccc3c2C1]

tert-butyl 7-bromo-3,4-dihydrobenzofuro[3,2-c]pyridine-2(1H)-carboxylate (220 mg, 0.62 mmol) and 4-(2-(5-chloropyridin-2-yl)ethyl)piperazin-2-one (150 mg, 0.62 mmol) were reacted according to Example 3 (step b) to provide the title compound (227 mg, 72%) as an off-white solid: ¹H NMR (500 MHz, CDCl₃) δ 8.51 (d, J=2.5 Hz, 1H), 7.60 (dd, J=8.5, 2.5 Hz, 1H), 7.41 (d, J=8.0 Hz, 1H), 7.37 (d, J=2.0 Hz, 1H), 7.17 (d, J=8.0 Hz, 1H), 7.14 (d, J=8.0 Hz, 1H), 4.54 (s, 2H), 3.82 (s, 2H), 3.71 (t, J=5.5 Hz, 2H), 3.41 (s, 2H), 3.02 (t, J=7.5 Hz, 2H), 2.92-2.89 (m, 4H), 2.85 (s, 2H), 1.50 (s, 9H); ESI MS m/z 511 [M+H]⁺.

### e) 4-(2-(5-Chloropyridin-2-yl)ethyl)-1-(1,2,3,4-tetrahydrobenzofuro[3,2-c]pyridin-7-yl)piperazin-2-one

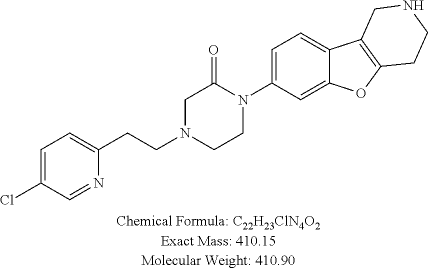 [SMILES: O=C1CN(CCc2ccc(Cl)cn2)CCN1c1ccc2c3c(oc2c1)CCNC3]

A solution of tert-butyl 7-(4-(2-(5-chloropyridin-2-yl)ethyl)-2-oxopiperazin-1-yl)-3,4-dihydrobenzofuro[3,2-c]pyridine-2(1H)-carboxylate (225 mg, 0.440 mmol) in MeOH (5.0 mL) was treated with 2 N HCl in Et₂O (10 mL), and the resulting solution was stirred at ambient temperature for 16 h and then concentrated in vacuo. The resultant HCl salt was converted to the corresponding free base using an SCX-2 cartridge to provide the title compound (177 mg, 98%) as an off-white solid: ¹H NMR (500 MHz, CDCl₃) δ 8.51 (d, J=2.5 Hz, 1H), 7.60 (dd, J=8.0, 2.5 Hz, 1H), 7.38-7.36 (m, 2H), 7.17 (d, J=8.0 Hz, 1H), 7.12 (dd, J=8.3, 1.8 Hz, 1H), 3.96 (t, J=2.0 Hz, 2H), 3.71 (t, J=5.5 Hz, 2H), 3.41 (s, 2H), 3.24 (t, J=5.8 Hz, 2H), 3.02 (t, J=7.5 Hz, 2H), 2.92-2.89 (m, 4H), 2.80-2.77 (m, 2H); ESI MS m/z 411 [M+H]⁺.

### f) 4-(2-(5-Chloropyridin-2-yl)ethyl)-1-(1,2,3,4-tetrahydrobenzofuro[3,2-c]pyridin-7-yl)piperazin-2-one hydrochloride

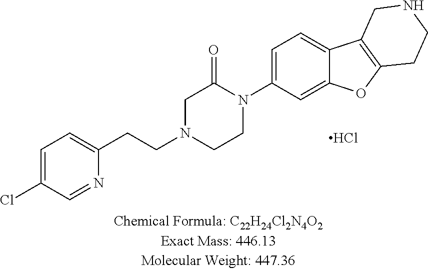 [SMILES: O=C1CN(CCc2ccc(Cl)cn2)CCN1c1ccc2c3c(oc2c1)CCNC3 ; Cl]

A solution of 4-(2-(5-chloropyridin-2-yl)ethyl)-1-(1,2,3,4-tetrahydrobenzofuro[3,2-c]pyridin-7-yl)piperazin-2-one (174 mg, 0.423 mmol) in MeOH (5.0 mL) was treated with 2 N HCl in Et₂O (0.21 mL, 0.42 mmol), and the resulting suspension was concentrated. The solid was dissolved in H₂O (3 mL), frozen, and lyophilized overnight to provide the title compound (199 mg, quant. yield) as a white solid: mp 220-223° C.; ¹H NMR (500 MHz, CD₃OD) δ 8.55 (d, J=2.0 Hz, 1H), 7.84 (dd, J=8.0, 2.5 Hz, 1H), 7.62 (d, J=8.5 Hz, 1H), 7.57 (d, J=1.0 Hz, 1H), 7.43 (d, J=8.5 Hz, 1H), 7.29 (dd, J=8.0, 1.5 Hz, 1H), 4.45 (s, 2H), 3.96 (s, 2H), 3.90 (br s, 2H), 3.69 (t, J=6.3 Hz, 2H), 3.54 (br s, 2H), 3.47 (br s, 2H), 3.28-3.26 (m, 2H), 3.19 (t, J=6.0 Hz, 2H); ESI MS m/z 411 [M+H]⁺; HPLC (Method A)>99% (AUC), t_{R}=10.5 min.

### Example 92

### Preparation of 4-(Benzyloxy)-1-(2-(2-fluoroethyl)-1,2,3,4-tetrahydrobenzofuro[3,2-c]pyridin-7-yl)pyridin-2(1H)-one hydrochloride

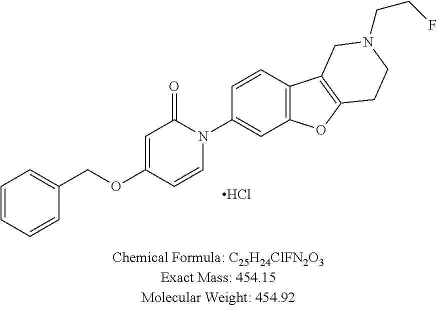 [SMILES: O=c1cc(OCc2ccccc2)ccn1-c1ccc2c3c(oc2c1)CCN(CCF)C3 ; Cl]

4-(Benzyloxy)-1-(1,2,3,4-tetrahydrobenzofuro[3,2-c]pyridin-7-yl)pyridin-2(1H)-one hydrochloride (100 mg, 0.245 mmol) was alkylated according to Example 59 (step a) and converted to the hydrochloride according to Example 12 (step d) to provide the title compound (40 mg, 36%) as a white solid: ¹H NMR (300 MHz, CD₃OD) δ 7.75-7.62 (m, 3H), 7.52-7.27 (m, 6H), 6.41 (dd, J=7.6, 2.7 Hz, 1H), 6.21 (d, J=2.6 Hz, 1H), 5.22 (s, 2H), 5.07 (t, J=4.4 Hz, 1H), 4.98-4.87 (m, 1H), 4.86-4.76 (m, 1H), 4.60-4.50 (m 1H), 4.10-3.95 (m, 1H), 3.90-3.84 (m, 1H), 3.81-3.75 (m, 2H), 3.41-3.23 (m, 2H); ESI MS m/z 419 [M+H]⁺; HPLC (Method A)>99% (AUC), t_{R}=14.7 min.

### Example 93

### Preparation of 4-(4-Chlorophenethyl)-1-(2-methyl-2,3,4,5-tetrahydro-1H-benzofuro[3,2-c]azepin-8-yl)piperazin-2-one hydrochloride

### a) 4-(4-Chlorophenethyl)-1-(2-methyl-2,3,4,5-tetrahydro-1H-benzofuro[3,2-c]azepin-8-yl)piperazin-2-one

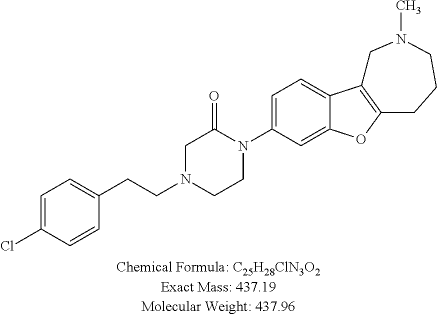 [SMILES: CN1CCCc2oc3cc(N4CCN(CCc5ccc(Cl)cc5)CC4=O)ccc3c2C1]

A mixture of 4-(4-chlorophenethyl)-1-(2,3,4,5-tetrahydro-1H-benzofuro[3,2-c]azepin-8-yl)piperazin-2-one hydrochloride (45 mg, 0.10 mmol) in dichloromethane (0.6 mL) and methanol (0.6 mL) was treated with 37% aqueous formaldehyde solution (20 μL, 0.24 mmol) followed by sodium triacetoxyborohydride (62 mg, 0.29 mmol). After stirring at ambient temperature for 1 h, the mixture was diluted with saturated aqueous NaHCO₃ solution (50 mL) and extracted with dichloromethane (5×50 mL). The combined organic extracts were dried over Na₂SO₄ and concentrated under reduced pressure. Purification by flash chromatography (silica gel, CH₂Cl₂/(90:9:1 CH₂Cl₂/MeOH/NH₄OH), 100:0 to 25:75) afforded the title compound (41 mg, 96%) as a white solid: ESI MS m/z 438 [M+H]⁺.

### b) 4-(4-Chlorophenethyl)-1-(2-methyl-2,3,4,5-tetrahydro-1H-benzofuro[3,2-c]azepin-8-yl)piperazin-2-one hydrochloride

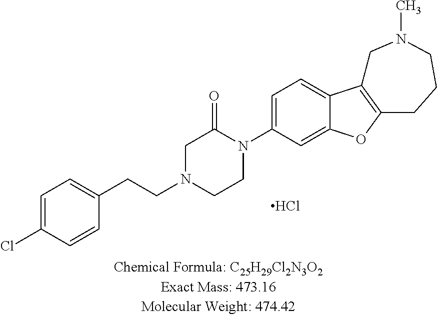 [SMILES: CN1CCCc2oc3cc(N4CCN(CCc5ccc(Cl)cc5)CC4=O)ccc3c2C1 ; Cl]

4-(4-Chlorophenethyl)-1-(2-methyl-2,3,4,5-tetrahydro-1H-benzofuro[3,2-c]azepin-8-yl)piperazin-2-one (41 mg, 0.094 mmol) was suspended in MeOH (5 mL) and treated with 2 N HCl in Et₂O (47 μL). The suspension was stirred at ambient temperature for 30 min. The solvent was removed, and the residue was co-evaporated with acetonitrile and lyophilized from acetonitrile-water to provide the title compound (45 mg, quant) as a white solid: ¹H NMR (500 MHz, DMSO-d₆) δ 10.54 (s, 1H), 7.65 (d, J=8.0 Hz, 1H), 7.56 (d, J=1.5 Hz, 1H), 7.38-7.24 (m, 5H), 4.71-4.37 (m, 2H), 3.79-3.41 (m, 5H), 3.27-3.21 (m, 2H), 3.07 (t, J=6.0 Hz, 2H), 2.92-2.77 (m, 8H), 2.23-2.132 (m, 2H); ESI MS m/z 438 [M+H]⁺; HPLC (Method B)>99% (AUC), t_{R}=12.0 min.

### Example 94

### Preparation of 4-(2-(5-Chloropyridin-2-yl)ethyl)-1-(2-methyl-2,3,4,5-tetrahydro-1H-benzofuro[3,2-c]azepin-8-yl)piperazin-2-one hydrochloride

### a) 4-(2-(5-Chloropyridin-2-yl)ethyl)-1-(2-methyl-2,3,4,5-tetrahydro-1H-benzofuro[3,2-c]azepin-8-yl)piperazin-2-one

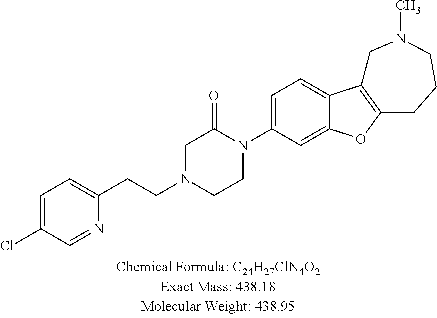 [SMILES: CN1CCCc2oc3cc(N4CCN(CCc5ccc(Cl)cn5)CC4=O)ccc3c2C1]

A mixture of 4-(2-(5-chloropyridin-2-yl)ethyl)-1-(2,3,4,5-tetrahydro-1H-benzofuro[3,2-c]azepin-8-yl)piperazin-2-one hydrochloride (48 mg, 0.10 mmol) in dichloromethane (0.7 mL) and methanol (0.7 mL) was treated with 37% aqueous formaldehyde solution (21 μL, 0.26 mmol) followed by sodium triacetoxyborohydride (66 mg, 0.31 mmol). After stirring at ambient temperature for 1 h, the mixture was diluted with saturated aqueous NaHCO₃ solution (50 mL) and extracted with dichloromethane (5×50 mL). The combined organic extracts were dried over Na₂SO₄ and concentrated under reduced pressure. Purification by flash chromatography (silica gel, CH₂Cl₂/(90:9:1 CH₂Cl₂/MeOH/NH₄OH), 100:0 to 25:75) afforded the title compound (43 mg, 94%) as a white solid: ESI MS m/z 439 [M+H]⁺.

### b) 4-(2-(5-Chloropyridin-2-yl)ethyl)-1-(2-methyl-2,3,4,5-tetrahydro-1H-benzofuro[3,2-c]azepin-8-yl)piperazin-2-one hydrochloride

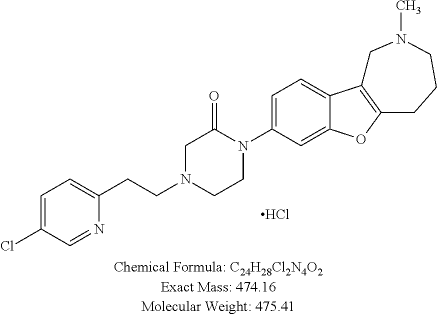 [SMILES: CN1CCCc2oc3cc(N4CCN(CCc5ccc(Cl)cn5)CC4=O)ccc3c2C1 ; Cl]

4-(2-(5-Chloropyridin-2-yl)ethyl)-1-(2-methyl-2,3,4,5-tetrahydro-1H-benzofuro[3,2-c]azepin-8-yl)piperazin-2-one (43 mg, 0.10 mmol) was suspended in MeOH (5 mL) and treated with 2 N HCl in Et₂O (49 μL, 0.098). The suspension was stirred at ambient temperature for 30 min. The solvent was removed, and the residue was co-evaporated with acetonitrile and lyophilized from acetonitrile-water to provide the title compound (47 mg, quant) as a white solid: ¹H NMR (500 MHz, DMSO-d₆) δ 8.41 (s, 1H), 7.72 (dd, J=8.5, 2.5 Hz, 1H), 7.55 (d, J=8.0 Hz, 1H), 7.40 (d, J=1.5 Hz, 1H), 7.32 (d, J=8.5 Hz, 1H), 7.16 (dd, J=8.0, 1.5 Hz, 1H), 4.59-4.44 (m, 2H), 3.91-3.25 (m, 7H), 3.18-2.98 (m, 11H), 2.38-2.10 (m, 2H); ESI MS m/z 439 [M+H]⁺; HPLC (Method B)>99% (AUC), t_{R}=10.7 min.

### Example 95

### Preparation of 4-(2-(5-Fluoropyridin-2-yl)ethyl)-1-(2-methyl-2,3,4,5-tetrahydro-1H-benzofuro[3,2-c]azepin-8-yl)piperazin-2-one hydrochloride

### a) 4-(2-(5-Fluoropyridin-2-yl)ethyl)-1-(2-methyl-2,3,4,5-tetrahydro-1H-benzofuro[3,2-c]azepin-8-yl)piperazin-2-one

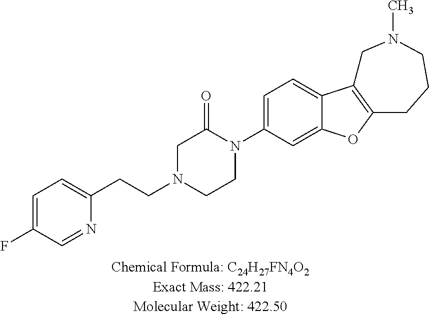 [SMILES: CN1CCCc2oc3cc(N4CCN(CCc5ccc(F)cn5)CC4=O)ccc3c2C1]

A mixture of 4-(2-(5-fluoropyridin-2-yl)ethyl)-1-(2,3,4,5-tetrahydro-1H-benzofuro[3,2-c]azepin-8-yl)piperazin-2-one hydrochloride (48 mg, 0.11 mmol) in dichloromethane (0.7 mL) and methanol (0.7 mL) was treated with 37% aqueous formaldehyde solution (22 μL, 0.27 mmol) followed by sodium triacetoxyborohydride (69 mg, 0.32 mmol). After stirring at ambient temperature for 1 h, the mixture was diluted with saturated aqueous NaHCO₃ solution (50 mL) and extracted with dichloromethane (5×50 mL). The combined organic extracts were dried over Na₂SO₄ and concentrated under reduced pressure. Purification by flash chromatography (silica gel, CH₂Cl₂/(90:9:1 CH₂Cl₂/MeOH/NH₄OH), 100:0 to 25:75) afforded the title compound (41 mg, 90%) as a white solid: ESI MS m/z 423 [M+H]⁺.

### b) 4-(2-(5-Fluoropyridin-2-yl)ethyl)-1-(2-methyl-2,3,4,5-tetrahydro-1H-benzofuro[3,2-c]azepin-8-yl)piperazin-2-one hydrochloride

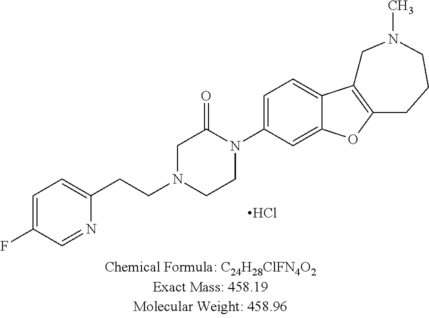 [SMILES: CN1CCCc2oc3cc(N4CCN(CCc5ccc(F)cn5)CC4=O)ccc3c2C1 ; Cl]

4-(2-(5-Fluoropyridin-2-yl)ethyl)-1-(2-methyl-2,3,4,5-tetrahydro-1H-benzofuro[3,2-c]azepin-8-yl)piperazin-2-one (41 mg, 0.10 mmol) was suspended in MeOH (5 mL) and treated with 2 N HCl in Et₂O (48 μL, 0.096 mmol). The suspension was stirred at ambient temperature for 30 min. The solvent was removed, and the residue was co-evaporated with acetonitrile and lyophilized from acetonitrile-water to provide the title compound (44 mg, quant) as a white solid: ¹H NMR (500 MHz, DMSO-d₆) δ 8.34 (s, 1H), 7.56 (d, J=8.5 Hz, 1H), 7.54-7.49 (m, 1H), 7.42 (d, J=1.5 Hz, 1H), 7.38-7.35 (m, 1H), 7.19 (dd, J=8.5, 1.5 Hz, 1H), 4.66-4.39 (m, 2H), 3.95-3.43 (m, 7H), 3.39-3.25 (m, 2H), 3.14-3.06 (m, 6H), 2.98 (s, 3H), 2.23-2.09 (m, 2H); ESI MS m/z 423 [M+H]⁺; HPLC (Method B)>99% (AUC), t_{R}=10.1 min.

### Example 96

### Preparation of 4-(4-Chlorophenethyl)-1-(2-isopropyl-2,3,4,5-tetrahydro-1H-benzofuro[3,2-c]azepin-8-yl)piperazin-2-one hydrochloride

### a) 4-(4-Chlorophenethyl)-1-(2-isopropyl-2,3,4,5-tetrahydro-1H-benzofuro[3,2-c]azepin-8-yl)piperazin-2-one

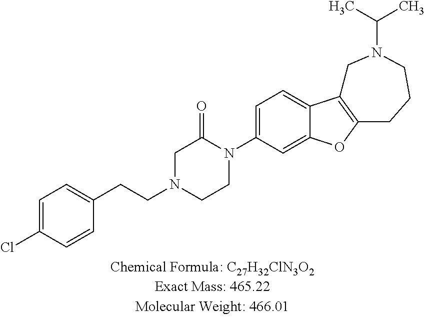 [SMILES: CC(C)N1CCCc2oc3cc(N4CCN(CCc5ccc(Cl)cc5)CC4=O)ccc3c2C1]

A mixture of 4-(4-chlorophenethyl)-1-(2,3,4,5-tetrahydro-1H-benzofuro[3,2-c]azepin-8-yl)piperazin-2-one hydrochloride (46 mg, 0.10 mmol) in dichloromethane (2 mL) and acetone (2 mL) was treated with acetic acid (0.2 mL). After stirring the mixture at ambient temperature for 5 min, 2-picoline borane complex (43 mg, 0.40 mmol) was added. The reaction mixture was heated at 50° C. for 2 h. The mixture was cooled, diluted with saturated aqueous NaHCO₃ solution (50 mL) and extracted with dichloromethane (5×50 mL). The combined organic extracts were dried over Na₂SO₄ and concentrated under reduced pressure. Purification by flash chromatography (silica gel, CH₂Cl₂/(90:9:1 CH₂Cl₂/MeOH/NH₄OH), 100:0 to 25:75) afforded the title compound (33 mg, 71%) as a white solid: ESI MS m/z 466 [M+H]⁺.

### b) 4-(4-Chlorophenethyl)-1-(2-isopropyl-2,3,4,5-tetrahydro-1H-benzofuro[3,2-c]azepin-8-yl)piperazin-2-one

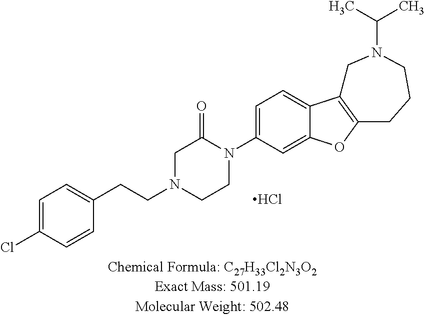 [SMILES: CC(C)N1CCCc2oc3cc(N4CCN(CCc5ccc(Cl)cc5)CC4=O)ccc3c2C1 ; Cl]

4-(4-Chlorophenethyl)-1-(2-isopropyl-2,3,4,5-tetrahydro-1H-benzofuro[3,2-c]azepin-8-yl)piperazin-2-one (33 mg, 0.071 mmol) was suspended in MeOH (5 mL) and treated with 2 N HCl in Et₂O (35 μL, 0.070 mmol). The suspension was stirred at ambient temperature for 30 min. The solvent was removed, and the residue was co-evaporated with acetonitrile and lyophilized from acetonitrile-water to provide the title compound (34 mg, 96%) as a white solid: ¹H NMR (500 MHz, DMSO-d₆) δ 7.61 (d, J=8.0 Hz, 1H), 7.43 (d, J=1.5 Hz, 1H), 7.26-7.18 (m, 5H), 4.56-4.46 (m, 2H), 3.90-3.40 (m, 9H), 3.20-2.85 (m, 7H), 2.32-2.05 (m, 2H), 1.40-1.35 (m, 6H); ESI MS m/z 466 [M+H]⁺; HPLC (Method B)>99% (AUC), t_{R}=12.3 min.

### Example 97

### Preparation of 4-(2-(5-Chloropyridin-2-yl)ethyl)-1-(2-isopropyl-2,3,4,5-tetrahydro-1H-benzofuro[3,2-c]azepin-8-yl)piperazin-2-one hydrochloride

### a) 4-(2-(5-Chloropyridin-2-yl)ethyl)-1-(2-isopropyl-2,3,4,5-tetrahydro-1H-benzofuro[3,2-c]azepin-8-yl)piperazin-2-one

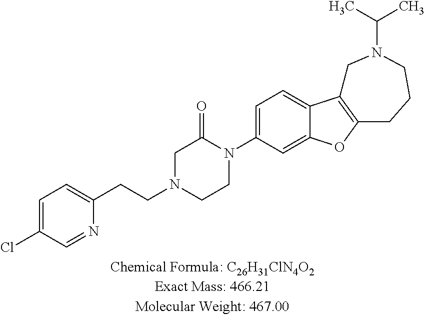 [SMILES: CC(C)N1CCCc2oc3cc(N4CCN(CCc5ccc(Cl)cn5)CC4=O)ccc3c2C1]

A mixture of 4-(2-(5-chloropyridin-2-yl)ethyl)-1-(2,3,4,5-tetrahydro-1H-benzofuro[3,2-c]azepin-8-yl)piperazin-2-one hydrochloride (46 mg, 0.10 mmol) in dichloromethane (2 mL) and acetone (2 mL) was treated with acetic acid (0.2 mL). After stirring the mixture at ambient temperature for 5 minutes, 2-picoline borane complex (43 mg, 0.40 mmol) was added. The reaction mixture was heated at 50° C. for 2 h. The mixture was cooled, diluted with saturated aqueous NaHCO₃ solution (50 mL) and extracted with dichloromethane (5×50 mL). The combined organic extracts were dried over Na₂SO₄ and concentrated under reduced pressure. Purification by flash chromatography (silica gel, CH₂Cl₂/(90:9:1 CH₂Cl₂/MeOH/NH₄OH), 100:0 to 25:75) afforded the title compound (32 mg, 69%) as a white solid: ESI MS m/z 467 [M+H]⁺.

### b) 4-(2-(5-Chloropyridin-2-yl)ethyl)-1-(2-isopropyl-2,3,4,5-tetrahydro-1H-benzofuro[3,2-c]azepin-8-yl)piperazin-2-one hydrochloride

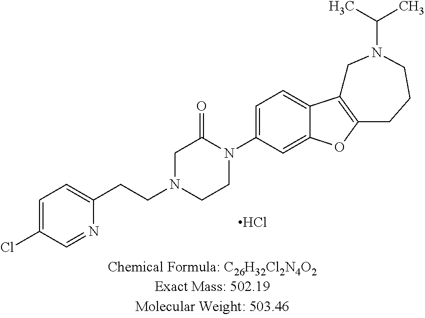 [SMILES: CC(C)N1CCCc2oc3cc(N4CCN(CCc5ccc(Cl)cn5)CC4=O)ccc3c2C1 ; Cl]

4-(2-(5-Chloropyridin-2-yl)ethyl)-1-(2-isopropyl-2,3,4,5-tetrahydro-1H-benzofuro[3,2-c]azepin-8-yl)piperazin-2-one (32 mg, 0.069 mmol) was suspended in MeOH (5 mL) and treated with 2 N HCl in Et₂O (34 μL, 0.068 mmol). The suspension was stirred at ambient temperature for 30 min. The solvent was removed, and the residue was co-evaporated with acetonitrile and lyophilized from acetonitrile-water to provide the title compound (35 mg, quant) as a white solid: ¹H NMR (500 MHz, DMSO-d₆) δ 8.41 (d, J=1.5 Hz, 1H), 7.72 (dd, J=8.5, 2.5 Hz, 1H), 7.59 (d, J=8.0 Hz, 1H), 7.40 (d, J=1.5 Hz, 1H), 7.31 (d, J=8.5 Hz, 1H), 7.17 (dd, J=8.0, 1.5 Hz, 1H), 4.55-4.45 (m, 2H), 3.80-3.40 (m, 8H), 3.20-2.90 (m, 8H), 2.35-2.00 (m, 2H), 1.39-1.34 (m, 6H); ESI MS m/z 467 [M+H]⁺; HPLC (Method B) 97.8% (AUC), t_{R}=11.0 min.

### Example 98

### Preparation of 4-(2-(5-Chloropyridin-2-yl)ethyl)-1-(2,3,4,5-tetrahydro-1H-benzofuro[2,3-d]azepin-8-yl)piperazin-2-one hydrochloride

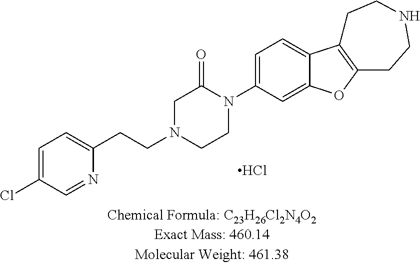 [SMILES: O=C1CN(CCc2ccc(Cl)cn2)CCN1c1ccc2c3c(oc2c1)CCNCC3 ; Cl]

4-(2-(5-Chloropyridin-2-yl)ethyl)-1-(2,3,4,5-tetrahydro-1H-benzofuro[2,3-d]azepin-8-yl)piperazin-2-one hydrochloride was prepared according to the procedure for Example 90 to provide the title compound as an off-white solid: ¹H NMR (500 MHz, CD₃OD) δ 8.39 (d, J=2.5 Hz, 1H), 7.71 (dd, J=8.5, 2.5 Hz, 1H), 7.47 (d, J=8.0 Hz, 1H), 7.33-7.30 (m, 2H), 7.10 (dd, J=8.0, 1.5 Hz, 1H), 3.69-3.67 (m, 2H), 3.45-3.38 (m, 6H), 3.24-2.23 (m, 2H), 3.04-2.94 (m, 8H); ESI MS m/z 425 [M+H]⁺; HPLC (Method B)>99% (AUC), t_{R}=10.6 min.

### Example 99

### Preparation of 4-(4-Chlorophenethyl)-1-(2,3,4,5-tetrahydro-1H-benzofuro[2,3-d]azepin-8-yl)piperazin-2-one hydrochloride

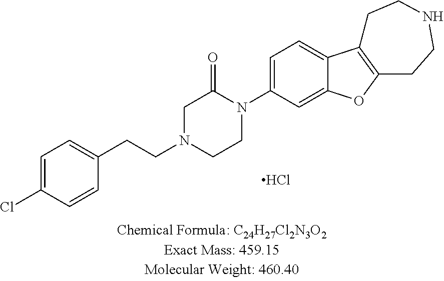 [SMILES: O=C1CN(CCc2ccc(Cl)cc2)CCN1c1ccc2c3c(oc2c1)CCNCC3 ; Cl]

4-(4-Chlorophenethyl)-1-(2,3,4,5-tetrahydro-1H-benzofuro[2,3-d]azepin-8-yl)piperazin-2-one hydrochloride was prepared according to the procedure for Example 89 to provide the title compound as an off-white solid: ¹H NMR (500 MHz, CD₃OD) δ 7.47 (d, J=8.5 Hz, 1H), 7.33 (d, J=2.0 Hz, 1H), 7.22-7.17 (m, 4H), 7.11 (dd, J=8.0, 1.5 Hz, 1H), 3.69-3.67 (m, 2H), 3.45-3.34 (m, 6H), 3.25-2.22 (m, 2H), 3.05-2.95 (m, 4H), 2.81-2.75 (m, 4H); ESI MS m/z 424 [M+H]⁺; HPLC (Method B)>99% (AUC), t_{R}=11.9 min.

### Example 100

### Preparation of 4-(2-(5-Fluoropyridin-2-yl)ethyl)-1-(2,3,4,5-tetrahydro-1H-benzofuro[3,2-c]azepin-8-yl)pyridin-2(1H)-one hydrochloride

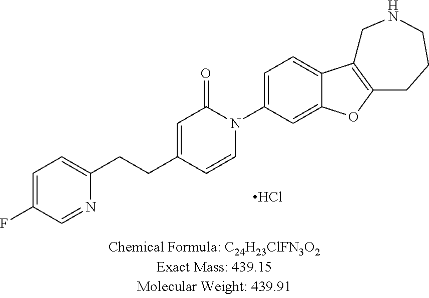 [SMILES: O=c1cc(CCc2ccc(F)cn2)ccn1-c1ccc2c3c(oc2c1)CCCNC3 ; Cl]

4-(2-(5-Fluoropyridin-2-yl)ethyl)-1-(2,3,4,5-tetrahydro-1H-benzofuro[3,2-c]azepin-8-yl)pyridin-2(1H)-one hydrochloride was prepared according to the procedure for Example 40 to provide the title compound as a white solid: ¹H NMR (500 MHz, DMSO-d₆) δ 9.39 (s, 2H), 8.50 (d, J=3.0 Hz, 1H), 7.77 (d, J=8.5 Hz, 1H), 7.69-7.63 (m, 2H), 7.58 (d, J=7.0 Hz, 1H), 7.43 (dd, J=8.5, 4.5 Hz, 1H), 7.26 (dd, J=8.0, 1.5 Hz, 1H), 6.31 (d, J=1.0 Hz, 1H), 6.27 (dd, J=7.0, 1.5 Hz, 1H), 4.40 (br s, 2H), 3.47 (br s, 2H), 3.12-3.06 (m, 4H), 2.91-2.88 (m, 2H), 2.11-2.09 (m, 2H); ESI MS m/z 404 [M+H]⁺; HPLC (Method B)>99% (AUC), t_{R}=12.1 min.

### Example 101

### Preparation of 4-(2-(5-Chloropyridin-2-yl)ethyl)-1-(1,2,3,4-tetrahydrobenzofuro[3,2-c]pyridin-7-yl)pyrimidin-2(1H)-one hydrochloride

### a) tert-Butyl 7-(4-(2-(5-chloropyridin-2-yl)ethyl)-2-oxopyrimidin-1(2H)-yl)-3,4-dihydrobenzofuro[3,2-c]pyridine-2(1H)-carboxylate

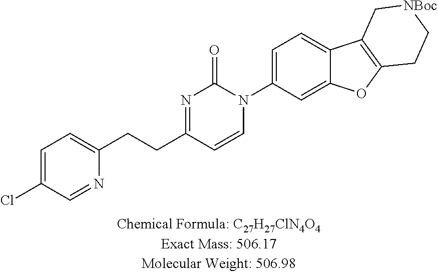 [SMILES: CC(C)(C)OC(=O)N1CCc2oc3cc(-n4ccc(CCc5ccc(Cl)cn5)nc4=O)ccc3c2C1]

tert-Butyl 7-bromo-3,4-dihydrobenzofuro[3,2-c]pyridine-2(1H)-carboxylate (180 mg, 0.51 mmol) and 4-(2-(5-chloropyridin-2-yl)ethyl)pyrimidin-2(1H)-one (120 mg, 0.51 mmol) were reacted according to Example 3 (step b) to provide the title compound (77 mg, 30%) as a yellow solid: ¹H NMR (500 MHz, CDCl₃) δ 8.50 (d, J=2.5 Hz, 1H), 7.62 (d, J=6.5 Hz, 1H), 7.58 (dd, J=8.5, 2.5 Hz, 1H), 7.51 (d, J=2.0 Hz, 1H), 7.49 (d, J=8.0 Hz, 1H), 7.23 (br s, 1H), 7.21 (d, J=8.0 Hz, 1H), 6.31 (d, J=7.0 Hz, 1H), 4.56 (s, 2H), 3.84 (s, 2H), 3.31 (t, J=7.8 Hz, 2H), 3.17 (t, J=7.8 Hz, 2H), 2.88 (s, 2H), 1.51 (s, 9H); ESI MS m/z 507 [M+H]⁺.

### b) 4-(2-(5-Chloropyridin-2-yl)ethyl)-1-(1,2,3,4-tetrahydrobenzofuro[3,2-c]pyridin-7-yl)pyrimidin-2(1H)-one

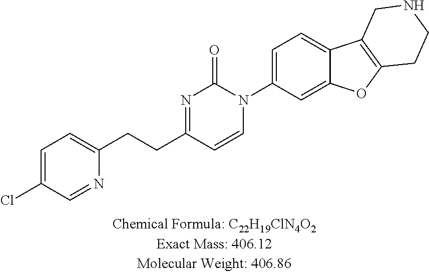 [SMILES: O=c1nc(CCc2ccc(Cl)cn2)ccn1-c1ccc2c3c(oc2c1)CCNC3]

A solution of tert-butyl 7-(4-(2-(5-chloropyridin-2-yl)ethyl)-2-oxopyrimidin-1(2H)-yl)-3,4-dihydrobenzofuro[3,2-c]pyridine-2(1H)-carboxylate (76 mg, 0.15 mmol) in MeOH (2.0 mL) was treated with 2 N HCl in Et₂O (3.5 mL), and the resulting solution was stirred at ambient temperature for 16 h and then concentrated in vacuo. The resultant HCl salt was converted to the corresponding free base using an SCX-2 cartridge to provide the title compound (61 mg, quant. yield) as an orange solid: ¹H NMR (500 MHz, CDCl₃) δ 8.50 (d, J=2.5 Hz, 1H), 7.62 (d, J=6.5 Hz, 1H), 7.58 (dd, J=8.5, 2.5 Hz, 1H), 7.49 (d, J=1.5 Hz, 1H), 7.45 (d, J=8.0 Hz, 1H), 7.22-7.19 (m, 2H), 6.30 (d, J=6.5 Hz, 1H), 3.99 (t, J=2.0 Hz, 2H), 3.31 (t, J=7.8 Hz, 2H), 3.26 (t, J=5.8 Hz, 2H), 3.16 (t, J=7.8 Hz, 2H), 2.83-2.80 (m, 2H); ESI MS m/z 407 [M+H]⁺.

### c) 4-(2-(5-Chloropyridin-2-yl)ethyl)-1-(1,2,3,4-tetrahydrobenzofuro[3,2-c]pyridin-7-yl)pyrimidin-2(1H)-one hydrochloride

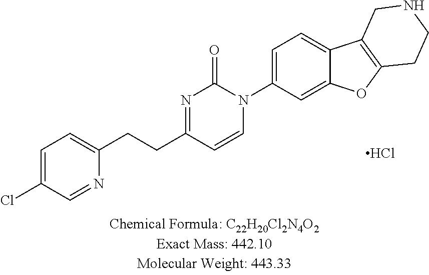 [SMILES: O=c1nc(CCc2ccc(Cl)cn2)ccn1-c1ccc2c3c(oc2c1)CCNC3 ; Cl]

A solution of 4-(2-(5-chloropyridin-2-yl)ethyl)-1-(1,2,3,4-tetrahydrobenzofuro[3,2-c]pyridin-7-yl)pyrimidin-2(1H)-one (60 mg, 0.15 mmol) in MeOH (5.0 mL) was treated with 2 N HCl in Et₂O (75 μL, 0.15 mmol) and the resulting suspension was concentrated. The solid was dissolved in H₂O (3 mL), frozen, and lyophilized overnight to provide the title compound (68 mg, quant. yield) as a light brown solid: mp 155-160° C.; ¹H NMR (500 MHz, DMSO-d₆) δ 9.79 (s, 2H), 8.57 (d, J=2.5 Hz, 1H), 8.29 (d, J=6.5 Hz, 1H), 7.90 (dd, J=8.5, 2.5 Hz, 1H), 7.82 (d, J=1.5 Hz, 1H), 7.74 (d, J=8.0 Hz, 1H), 7.45 (d, J=8.5 Hz, 1H), 7.38 (dd, J=8.3, 1.8 Hz, 1H), 6.66 (d, J=7.0 Hz, 1H), 4.35 (s, 2H), 3.56-3.53 (m, 2H), 3.23-3.21 (m, 2H), 3.15-3.12 (m, 4H); ESI MS m/z 407 [M+H]⁺; HPLC (Method A) 97.6% (AUC), t_{R}=11.7 min.

### Example 102

### Preparation of 4-(2-(5-Fluoropyridin-2-yl)ethyl)-1-(1,2,3,4-tetrahydrobenzofuro[3,2-c]pyridin-7-yl)pyridin-2(1H)-one hydrochloride

### a) 5-Fluoro-2-(2-(tributylstannyl)vinyl)pyridine

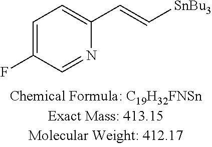

A solution of 2-bromo-5-fluoropyridine (1.14 g, 6.35 mmol) and trans-1,2-bis(tri-n-butylstannyl)ethylene (5.00 g, 8.25 mmol) in anhydrous toluene (20 mL) was degassed with argon for several minutes. Tetrakis(triphenylphosphine)palladium(0) (370 mg, 0.32 mmol) was added and, after degassing briefly with argon again, the flask was sealed, and the reaction was heated to 110° C. and stirred for 2 h. After cooling to rt, the reaction mixture was concentrated in vacuo. Flash chromatography (120 g ISCO Gold column, 0%-5% ethyl acetate/hexanes) provided the title compound (1.07 g, 41%) as a light yellow oil: ¹H NMR (500 MHz, CDCl₃) δ 8.41 (s, 1H), 7.38-7.35 (m, 2H), 7.26 (d, J=19.5 Hz, 1H), 6.98 (d, J=19.5 Hz, 1H), 1.58-1.51 (m, 6H), 1.37-1.30 (m, 6H), 0.99 (t, J=8.3 Hz, 6H), 0.90 (t, J=7.3 Hz, 9H).

### b) 4-(2-(5-Fluoropyridin-2-yl)vinyl)-2-methoxypyridine

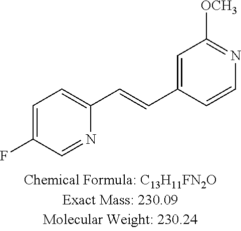 [SMILES: COc1cc(/C=C/c2ccc(F)cn2)ccn1]

A solution of 5-fluoro-2-(2-(tributylstannyl)vinyl)pyridine (1.06 g, 2.57 mmol) and 4-bromo-2-methoxypyridine (0.3 mL, 2.3 mmol) in anhydrous toluene (15 mL) was degassed for several minutes with argon.

Tetrakis(triphenylphosphine)palladium(0) (270 mg, 0.23 mmol) was added and, after degassing briefly with argon again, the flask was sealed, and the reaction was heated to 135° C. and stirred for 16 h. After cooling to rt, the reaction mixture was concentrated in vacuo. Flash chromatography (120 g ISCO Gold column, 10%-80% ethyl acetate/hexanes) provided the title compound (542 mg, 92%) as an off-white solid: ¹H NMR (500 MHz, CDCl₃) δ 8.49 (t, J=1.8 Hz, 1H), 8.15 (d, J=5.5 Hz, 1H), 7.43 (d, J=16.0 Hz, 1H), 7.42-7.40 (m, 2H), 7.26 (d, J=16.0 Hz, 1H), 7.05 (dd, J=5.5, 1.0 Hz, 1H), 6.83 (s, 1H), 3.96 (s, 3H); ESI MS m/z 231 [M+H]⁺.

### c) 4-(2-(5-Fluoropyridin-2-yl)ethyl)-2-methoxypyridine

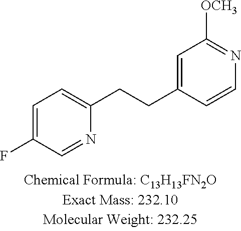 [SMILES: COc1cc(CCc2ccc(F)cn2)ccn1]

A solution of 4-(2-(5-fluoropyridin-2-yl)vinyl)-2-methoxypyridine (536 mg, 2.33 mmol) in methanol (30 mL) was degassed with argon for several minutes and then treated with 10% palladium on carbon (200 mg). The mixture was subjected to vacuum and hydrogen backfill three times and was stirred under hydrogen (balloon pressure) for 16 h. LC-MS indicated some starting material remained so additional 10% palladium on carbon (100 mg) was added and the reaction was stirred for 8 h. The reaction mixture was filtered through Celite and then concentrated in vacuo, providing the title compound (498 mg, 92%) as a colorless oil: ¹H NMR (500 MHz, CDCl₃) δ 8.41 (d, J=3.0 Hz, 1H), 8.04 (d, J=5.5 Hz, 1H), 7.28 (td, J=8.3, 2.8 Hz, 1H), 7.05 (dd, J=8.5, 4.5 Hz, 1H), 6.69 (dd, J=5.0, 1.0 Hz, 1H), 6.55 (s, 1H), 3.91 (s, 3H), 3.09-3.05 (m, 2H), 3.02-2.98 (m, 2H); ESI MS m/z 233 [M+H]⁺.

### d) 4-(2-(5-Fluoropyridin-2-yl)ethyl)pyridin-2(1H)-one

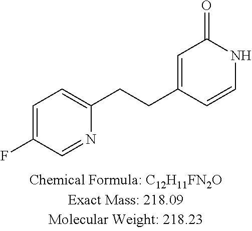 [SMILES: O=c1cc(CCc2ccc(F)cn2)cc[nH]1]

A mixture of 4-(2-(5-fluoropyridin-2-yl)ethyl)-2-methoxypyridine (495 mg, 2.13 mmol) and c. HCl (12 mL) was heated to reflux and stirred for 24 h. The reaction mixture was concentrated in vacuo, and the resultant solid was converted to the corresponding free base using an SCX-2 cartridge, providing the title compound (371 mg, 80%) as a white solid: ¹H NMR (500 MHz, CDCl₃) δ 12.54 (s, 1H), 8.40 (d, J=3.0 Hz, 1H), 7.30 (td, J=8.5, 3.0 Hz, 1H), 7.24 (d, J=6.5 Hz, 1H), 7.09 (dd, J=8.5, 4.5 Hz, 1H), 6.35 (s, 1H), 6.12 (dd, J=7.0, 1.5 Hz, 1H), 3.06 (t, J=7.8 Hz, 2H), 2.93 (t, J=7.8 Hz, 2H); ESI MS m/z 219 [M+H]⁺.

### e) tert-Butyl 7-(4-(2-(5-fluoropyridin-2-yl)ethyl)-2-oxopyridin-1(2H)-yl)-3,4-dihydrobenzofuro[3,2-c]pyridine-2(1H)-carboxylate

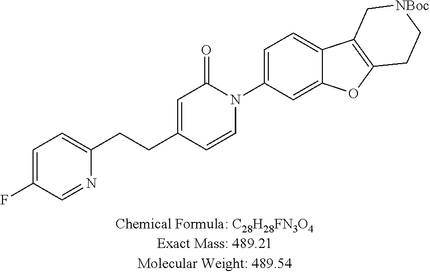 [SMILES: CC(C)(C)OC(=O)N1CCc2oc3cc(-n4ccc(CCc5ccc(F)cn5)cc4=O)ccc3c2C1]

tert-Butyl 7-bromo-3,4-dihydrobenzofuro[3,2-c]pyridine-2(1H)-carboxylate (180 mg, 0.511 mmol) and 4-(2-(5-fluoropyridin-2-yl)ethyl)pyridin-2(1H)-one (112 mg, 0.513 mmol) were reacted according to Example 3 (step b) to provide the title compound (153 mg, 61%) as a colorless oil: ¹H NMR (500 MHz, CDCl₃) δ 8.42 (d, J=3.0 Hz, 1H), 7.49-7.47 (m, 2H), 7.33 (td, J=8.5, 3.0 Hz, 1H), 7.28 (d, J=7.0 Hz, 1H), 7.21 (d, J=7.0 Hz, 1H), 7.16 (dd, J=8.5, 4.5 Hz, 1H), 6.48 (s, 1H), 6.12 (dd, J=7.0, 2.0 Hz, 1H), 4.57 (s, 2H), 3.84 (s, 2H), 3.11 (t, J=7.8 Hz, 2H), 2.95 (t, J=7.8 Hz, 2H), 2.87 (s, 2H), 1.51 (s, 9H); ESI MS m/z 490 [M+H]⁺.

### f) 4-(2-(5-Fluoropyridin-2-yl)ethyl)-1-(1,2,3,4-tetrahydrobenzofuro[3,2-c]pyridin-7-yl)pyridin-2(1H)-one

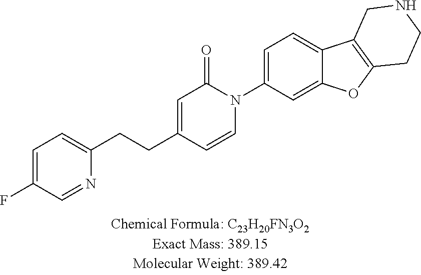 [SMILES: O=c1cc(CCc2ccc(F)cn2)ccn1-c1ccc2c3c(oc2c1)CCNC3]

A solution of tert-butyl 7-(4-(2-(5-fluoropyridin-2-yl)ethyl)-2-oxopyridin-1(2H)-yl)-3,4-dihydrobenzofuro[3,2-c]pyridine-2(1H)-carboxylate (152 mg, 0.310 mmol) in MeOH (4.0 mL) was treated with 2 N HCl in Et₂O (7 mL), and the resulting solution was stirred at ambient temperature for 16 h and then concentrated in vacuo. The resultant HCl salt was converted to the corresponding free base using an SCX-2 cartridge to provide the title compound (119 mg, 99%) as an off-white solid: ¹H NMR (500 MHz, CDCl₃) δ 8.42 (d, J=3.0 Hz, 1H), 7.45-7.43 (m, 2H), 7.33 (td, J=8.5, 3.0 Hz, 1H), 7.29 (d, J=7.0 Hz, 1H), 7.18 (dd, J=8.3, 1.8 Hz, 1H), 7.16 (dd, J=9.0, 4.5 Hz, 1H), 6.48 (d, J=1.0 Hz, 1H), 6.11 (dd, J=7.0, 2.0 Hz, 1H), 3.99 (t, J=1.8 Hz, 2H), 3.26 (t, J=5.8 Hz, 2H), 3.11 (t, J=7.8 Hz, 2H), 2.95 (t, J=7.8 Hz, 2H), 2.82-2.80 (m, 2H); ESI MS m/z 390 [M+H]⁺.

### g) 4-(2-(5-Fluoropyridin-2-yl)ethyl)-1-(1,2,3,4-tetrahydrobenzofuro[3,2-c]pyridin-7-yl)pyridin-2(1H)-one hydrochloride

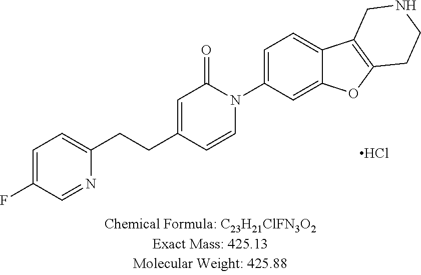 [SMILES: O=c1cc(CCc2ccc(F)cn2)ccn1-c1ccc2c3c(oc2c1)CCNC3 ; Cl]

A solution of 4-(2-(5-fluoropyridin-2-yl)ethyl)-1-(1,2,3,4-tetrahydrobenzofuro[3,2-c]pyridin-7-yl)pyridin-2(1H)-one (119 mg, 0.306 mmol) in MeOH (5.0 mL) was treated with 2 N HCl in Et₂O (0.16 mL, 0.31 mmol), and the resulting suspension was concentrated. The solid was dissolved in H₂O (3 mL), frozen, and lyophilized overnight to provide the title compound (139 mg, quant. yield) as a light yellow solid: mp 165-169° C.; ¹H NMR (500 MHz, DMSO-d₆) δ 9.64 (s, 2H), 8.54 (d, J=2.5 Hz, 1H), 7.74-7.69 (m, 3H), 7.59 (d, J=7.0 Hz, 1H), 7.46 (dd, J=9.0, 4.5 Hz, 1H), 7.28 (dd, J=8.3, 1.8 Hz, 1H), 6.32 (s, 1H), 6.28 (dd, J=7.0, 2.0 Hz, 1H), 4.35 (s, 2H), 3.56-3.53 (m, 2H), 3.13-3.08 (m, 4H), 2.90 (t, J=8.0 Hz, 2H); ESI MS m/z 390 [M+H]⁺; HPLC (Method A)>99% (AUC), t_{R}=11.8 min.

### Example 103

### Preparation of 4-(2-(5-Chloropyridin-2-yl)ethyl)-1-(2,3,4,5-tetrahydro-1H-benzofuro[3,2-c]azepin-8-yl)pyrimidin-2(1H)-one hydrochloride

### a) tert-Butyl 8-(4-(2-(5-chloropyridin-2-yl)ethyl)-2-oxopyrimidin-1(2H)-yl)-4,5-dihydro-1H-benzofuro[3,2-c]azepine-2(3H)-carboxylate

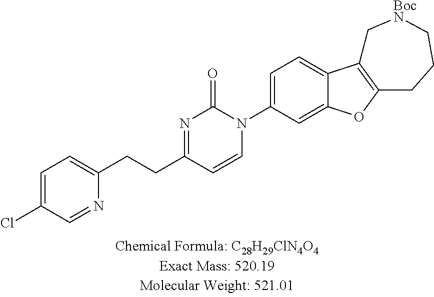 [SMILES: CC(C)(C)OC(=O)N1CCCc2oc3cc(-n4ccc(CCc5ccc(Cl)cn5)nc4=O)ccc3c2C1]

A mixture of 4-(2-(5-chloropyridin-2-yl)ethyl)pyrimidin-2(1H)-one (140 mg, 0.59 mmol), tert-butyl 8-bromo-4,5-dihydro-1H-benzofuro[3,2-c]azepine-2(3H)-carboxylate (167 mg, 0.456 mmol) and Cs₂CO₃ (193 mg, 0.592 mmol) in toluene (7 mL) in a sealed tube was degassed with a nitrogen stream for 10 min. Trans-N,N′-dimethylcyclohexane-1,2-diamine (97 mg, 0.68 mmol) and CuI (130 mg, 0.68 mmol) were added, and the mixture was degassed for another 2 min. The tube was sealed, and the mixture was heated at 110° C. for 12 h. The mixture was cooled, diluted with 90:9:1 CH₂Cl₂/MeOH/NH₄OH (15 mL) and stirred for 1 h. Then 2:1 brine/NH₄OH (100 mL) was added, and the aqueous phase was extracted with dichloromethane (4×75 mL). The combined organic extracts were washed with 2:1 brine/NH₄OH (3×75 mL), dried over Na₂SO₄ and concentrated under reduced pressure. Purification by flash chromatography (silica gel, CH₂Cl₂/(90:9:1 CH₂Cl₂/MeOH/NH₄OH), 100:0 to 25:75) afforded the title compound (96 mg, 40%) as a tan solid: ESI MS m/z 521 [M+H]⁺.

### b) 4-(2-(5-Chloropyridin-2-yl)ethyl)-1-(2,3,4,5-tetrahydro-1H-benzofuro[3,2-c]azepin-8-yl)pyrimidin-2(1H)-one

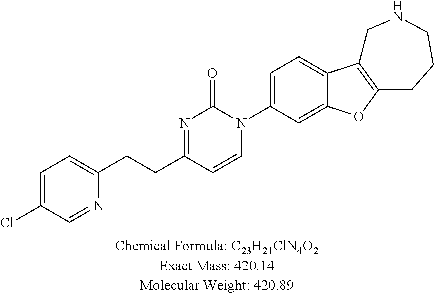 [SMILES: O=c1nc(CCc2ccc(Cl)cn2)ccn1-c1ccc2c3c(oc2c1)CCCNC3]

A solution of tert-butyl 8-(4-(2-(5-chloropyridin-2-yl)ethyl)-2-oxopyrimidin-1(2H)-yl)-4,5-dihydro-1H-benzofuro[3,2-c]azepine-2(3H)-carboxylate (96 mg, 0.18 mmol) in MeOH (4 mL) was treated with 2 N HCl in Et₂O (3 mL, 6 mmol), and the resulting solution was stirred at ambient temperature for 12 h. Saturated aqueous NaHCO₃ (40 mL) was added, and the mixture was extracted with dichloromethane (8×40 mL). The combined organic extracts were dried over Na₂SO₄ and concentrated under reduced pressure. Purification by flash chromatography (silica gel, CH₂Cl₂/(90:9:1 CH₂Cl₂/MeOH/NH₄OH), 100:0 to 10:90) afforded the title compound (62 mg, 78%) as a light yellow solid: ESI MS m/z 421 [M+H]⁺.

### c) 4-(2-(5-Chloropyridin-2-yl)ethyl)-1-(2,3,4,5-tetrahydro-1H-benzofuro[3,2-c]azepin-8-yl)pyrimidin-2(1H)-one hydrochloride

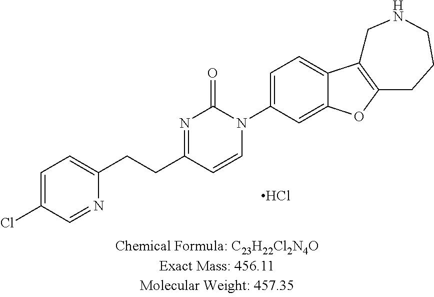 [SMILES: O=c1nc(CCc2ccc(Cl)cn2)ccn1-c1ccc2c3c(oc2c1)CCCNC3 ; Cl]

4-(2-(5-Chloropyridin-2-yl)ethyl)-1-(2,3,4,5-tetrahydro-1H-benzofuro[3,2-c]azepin-8-yl)pyrimidin-2(1H)-one (62 mg, 0.15 mmol) was suspended in MeOH (4 mL) and treated with 2 N HCl in Et₂O (74 μL, 0.15 mmol). The suspension was stirred at ambient temperature for 30 min. The solvent was removed, and the residue was co-evaporated with acetonitrile and lyophilized from acetonitrile-water to provide the title compound (68 mg, quant) as a white solid: ¹H NMR (500 MHz, DMSO-d₆) δ 9.37 (s, 2H), 8.55 (d, J=2.5 Hz, 1H), 8.10 (d, J=6.5 Hz, 1H), 7.86 (dd, J=8.0, 2.5 Hz, 1H), 7.79 (d, J=8.5 Hz, 1H), 7.73 (d, J=2.0 Hz, 1H), 7.42 (d, J=8.0 Hz, 1H), 7.34 (dd, J=8.5, 2.0 Hz, 1H), 6.53 (d, J=8.0 Hz, 1H), 4.41 (s, 2H), 3.47 (s, 2H), 3.20-3.06 (m, 6H), 2.09 (s, 2H); ESI MS m/z 421 [M+H]⁺; HPLC (Method B)>99% (AUC), t_{R}=11.9 min.

### Example 104

### Preparation of 4-Phenethyl-1-(1,2,3,4-tetrahydrobenzofuro[3,2-c]pyridin-7-yl)pyridin-2(1H)-one hydrochloride

### a) 4-Phenethylpyridin-2(1H)-one

CAS Registry Number 16097-16-8

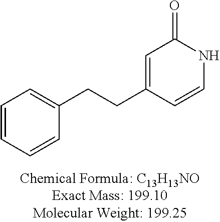 [SMILES: O=c1cc(CCc2ccccc2)cc[nH]1]

This compound was prepared in accordance with the procedure described in PCT Publication No. WO 2009/089482 to Guzzo et al., which is hereby incorporated by reference in its entirety.

### b) tert-Butyl 7-(2-oxo-4-phenethylpyridin-1(2H)-yl)-3,4-dihydrobenzofuro[3,2-c]pyridine-2(1H)-carboxylate

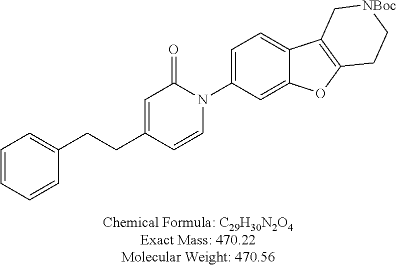 [SMILES: CC(C)(C)OC(=O)N1CCc2oc3cc(-n4ccc(CCc5ccccc5)cc4=O)ccc3c2C1]

tert-Butyl 7-bromo-3,4-dihydrobenzofuro[3,2-c]pyridine-2(1H)-carboxylate (200 mg, 0.568 mmol) and 4-phenethylpyridin-2(1H)-one (114 mg, 0.572 mmol) were reacted according to Example 3 (step b) to provide the title compound (213 mg, 79%) as a light yellow solid: ¹H NMR (500 MHz, CDCl₃) δ 7.49-7.47 (m, 2H), 7.33-7.27 (m, 3H), 7.24-7.21 (m, 4H), 6.50 (d, J=1.0 Hz, 1H), 6.10 (dd, J=7.0, 2.0 Hz, 1H), 4.57 (s, 2H), 3.84 (s, 2H), 2.97-2.94 (m, 2H), 2.88 (s, 2H), 2.83-2.80 (m, 2H), 1.51 (s, 9H); ESI MS m/z 471 [M+H]⁺.

### c) 4-Phenethyl-1-(1,2,3,4-tetrahydrobenzofuro[3,2-c]pyridin-7-yl)pyridin-2(1H)-one

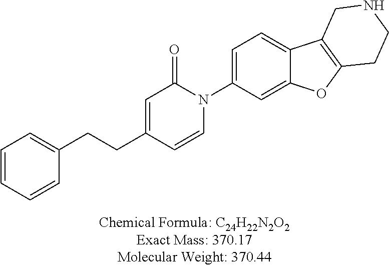 [SMILES: O=c1cc(CCc2ccccc2)ccn1-c1ccc2c3c(oc2c1)CCNC3]

A solution of tert-butyl 7-(2-oxo-4-phenethylpyridin-1(2H)-yl)-3,4-dihydrobenzofuro[3,2-c]pyridine-2(1H)-carboxylate (209 mg, 0.444 mmol) in MeOH (5.0 mL) was treated with 2 N HCl in Et₂O (10 mL), and the resulting solution was stirred at ambient temperature for 16 h and then concentrated in vacuo. The resultant HCl salt was converted to the corresponding free base using an SCX-2 cartridge to provide the title compound (167 mg, quant. yield) as a colorless oil: ¹H NMR (500 MHz, CDCl₃) δ 7.46 (d, J=1.5 Hz, 1H), 7.44 (d, J=8.0 Hz, 1H), 7.33-7.28 (m, 3H), 7.24-7.21 (m, 3H), 7.19 (dd, J=8.3, 1.8 Hz, 1H), 6.50 (d, J=1.0 Hz, 1H), 6.09 (dd, J=7.0, 2.0 Hz, 1H), 4.00 (t, J=1.8 Hz, 2H), 3.26 (t, J=5.8 Hz, 2H), 2.97-2.94 (m, 2H), 2.83-2.80 (m, 4H); ESI MS m/z 371 [M+H]⁺.

### d) 4-Phenethyl-1-(1,2,3,4-tetrahydrobenzofuro[3,2-c]pyridin-7-yl)pyridin-2(1H)-one hydrochloride

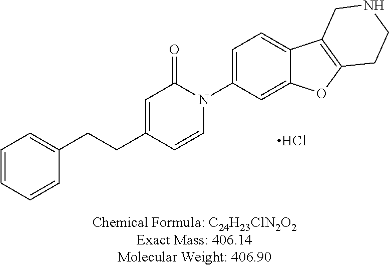 [SMILES: O=c1cc(CCc2ccccc2)ccn1-c1ccc2c3c(oc2c1)CCNC3 ; Cl]

A solution of 4-phenethyl-1-(1,2,3,4-tetrahydrobenzofuro[3,2-c]pyridin-7-yl)pyridin-2(1H)-one (165 mg, 0.445 mmol) in MeOH (5.0 mL) was treated with 2 N HCl in Et₂O (0.22 mL, 0.45 mmol) and the resulting suspension was concentrated. The solid was suspended in H₂O (3 mL), frozen, and lyophilized overnight to provide the title compound (178 mg, 97%) as an off-white solid: mp 283-289° C.; ¹H NMR (500 MHz, DMSO-d₆) δ 9.52 (s, 2H), 7.71 (s, 1H), 7.70 (d, J=7.0 Hz, 1H), 7.60 (d, J=7.0 Hz, 1H), 7.33-7.28 (m, 5H), 7.22-7.19 (m, 1H), 6.33 (s, 1H), 6.31 (dd, J=7.0, 2.0 Hz, 1H), 4.36 (s, 2H), 3.56 (s, 2H), 3.12 (t, J=6.0 Hz, 2H), 2.92 (t, J=7.8 Hz, 2H), 2.79 (t, J=8.0 Hz, 2H); ESI MS m/z 371 [M+H]⁺; HPLC (Method A)>99% (AUC), t_{R}=14.1 min.

### Example 105

### Preparation of 4-(2-(5-Chloropyridin-2-yl)ethyl)-1-(1,2,3,4-tetrahydrobenzofuro[3,2-c]pyridin-7-yl)pyridin-2(1H)-one hydrochloride

### a) 5-Chloro-2-(2-(tributylstannyl)vinyl)pyridine

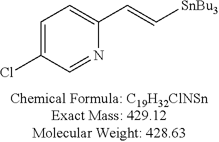

A solution of 2-bromo-5-chloropyridine (1.47 g, 7.62 mmol) and trans-1,2-bis(tri-n-butylstannyl)ethylene (6.00 g, 9.90 mmol) in anhydrous toluene (44 mL) was degassed with argon for several minutes. Tetrakis(triphenylphosphine)palladium(0) (440 mg, 0.38 mmol) was added and, after degassing briefly with argon again, the flask was sealed, and the reaction was heated to 110° C. and stirred for 1 h. After cooling to rt, the reaction mixture was concentrated in vacuo. Flash chromatography (120 g ISCO Gold column, 0%-5% ethyl acetate/hexanes) provided the title compound (1.42 g, 43%) as a light yellow oil: ¹H NMR (500 MHz, CDCl₃) δ 8.50 (d, J=2.5 Hz, 1H), 7.61 (dd, J=8.5, 2.5 Hz, 1H), 7.36 (d, J=19.5 Hz, 1H), 7.32 (d, J=8.5 Hz, 1H), 6.96 (d, J=19.5 Hz, 1H), 1.57-1.51 (m, 6H), 1.37-1.30 (m, 6H), 1.01-0.97 (m, 6H), 0.90 (t, J=7.3 Hz, 9H).

### b) 4-(2-(5-Chloropyridin-2-yl)vinyl)-2-methoxypyridine

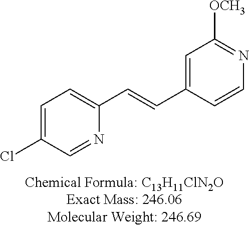 [SMILES: COc1cc(/C=C/c2ccc(Cl)cn2)ccn1]

A solution of 5-chloro-2-(2-(tributylstannyl)vinyl)pyridine (1.41 g, 3.29 mmol) and 4-bromo-2-methoxypyridine (0.37 mL, 3.0 mmol) in anhydrous toluene (19 mL) was degassed for several minutes with argon.

Tetrakis(triphenylphosphine)palladium(0) (347 mg, 0.30 mmol) was added and, after degassing briefly with argon again, the flask was sealed and the reaction was heated to 135° C. and stirred for 16 h. After cooling to rt, the reaction mixture was concentrated in vacuo. Flash chromatography (120 g ISCO Gold column, 7%-60% ethyl acetate/hexanes) provided the title compound (720 mg, 89%) as an off-white solid: ¹H NMR (500 MHz, CDCl₃) δ 8.57 (d, J=2.0 Hz, 1H), 8.16 (d, J=5.0 Hz, 1H), 7.67 (dd, J=8.5, 2.5 Hz, 1H), 7.50 (d, J=16.0 Hz, 1H), 7.35 (d, J=8.5 Hz, 1H), 7.24 (d, J=16.0 Hz, 1H), 7.05 (dd, J=5.3, 1.3 Hz, 1H), 6.84 (s, 1H), 3.96 (s, 3H); ESI MS m/z 247 [M+H]⁺.

### c) 4-(2-(5-Chloropyridin-2-yl)ethyl)-2-methoxypyridine

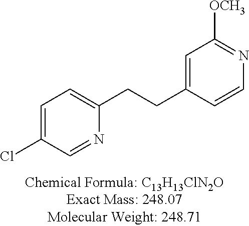 [SMILES: COc1cc(CCc2ccc(Cl)cn2)ccn1]

A solution of 4-(2-(5-chloropyridin-2-yl)vinyl)-2-methoxypyridine (610 mg, 2.47 mmol) in ethyl acetate (50 mL) was degassed with argon for several minutes 241- and then treated with zinc bromide (111 mg, 0.493 mmol) and 10% palladium on carbon (220 mg). The mixture was subjected to vacuum and hydrogen backfill three times and was stirred under hydrogen (balloon pressure) for 16 h. LC-MS indicated some starting material remained so additional 10% palladium on carbon (100 mg) was added, and the reaction was stirred for 20 h. The reaction mixture was filtered through Celite and then concentrated in vacuo to provide the title compound (330 mg, 54%) as a colorless oil: ¹H NMR (500 MHz, CDCl₃) δ 8.51 (d, J=2.5 Hz, 1H), 8.04 (d, J=5.5 Hz, 1H), 7.54 (dd, J=8.5, 2.5 Hz, 1H), 7.02 (d, J=8.0 Hz, 1H), 6.69 (dd, J=5.3, 1.3 Hz, 1H), 6.55 (d, J=1.0 Hz, 1H), 3.91 (s, 3H), 3.08-3.05 (m, 2H), 3.02-2.98 (m, 2H); ESI MS m/z 249 [M+H]⁺.

### d) 4-(2-(5-Chloropyridin-2-yl)ethyl)pyridin-2(1H)-one

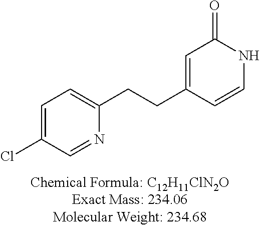 [SMILES: O=c1cc(CCc2ccc(Cl)cn2)cc[nH]1]

A mixture of 4-(2-(5-chloropyridin-2-yl)ethyl)-2-methoxypyridine (326 mg, 1.31 mmol) and c. HCl (15 mL) was heated to reflux and stirred for 24 h. The reaction mixture was concentrated in vacuo and the resultant solid was converted to the corresponding free base using an SCX-2 cartridge, providing the title compound (284 mg, 92%) as a white solid: ¹H NMR (500 MHz, CDCl₃) δ 12.53 (s, 1H), 8.50 (d, J=1.5 Hz, 1H), 7.26 (dd, J=8.5, 2.0 Hz, 1H), 7.24 (d, J=6.5 Hz, 1H), 7.05 (d, J=8.5 Hz, 1H), 6.35 (s, 1H), 6.12 (d, J=6.5 Hz, 1H), 3.06 (t, J=7.8 Hz, 2H), 2.93 (t, J=7.8 Hz, 2H); ESI MS m/z 235 [M+H]⁺.

### e) tert-Butyl 7-(4-(2-(5-chloropyridin-2-yl)ethyl)-2-oxopyridin-1(2H)-yl)-3,4-dihydrobenzofuro[3,2-c]pyridine-2(1H)-carboxylate

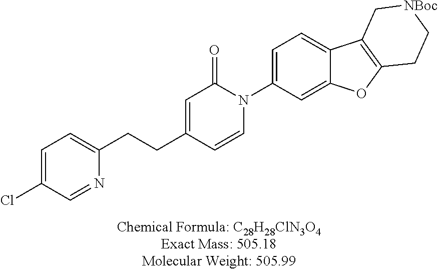 [SMILES: CC(C)(C)OC(=O)N1CCc2oc3cc(-n4ccc(CCc5ccc(Cl)cn5)cc4=O)ccc3c2C1]

tert-Butyl 7-bromo-3,4-dihydrobenzofuro[3,2-c]pyridine-2(1H)-carboxylate (151 mg, 0.429 mmol) and 4-(2-(5-chloropyridin-2-yl)ethyl)pyridin-2(1H)-one (100 mg, 0.426 mmol) were reacted according to Example 3 (step b) to provide the title compound (191 mg, 88%) as a light yellow solid: ¹H NMR (500 MHz, CDCl₃) δ 8.52 (d, J=2.5 Hz, 1H), 7.59 (dd, J=8.5, 2.5 Hz, 1H), 7.49-7.46 (m, 2H), 7.28 (d, J=7.0 Hz, 1H), 7.21 (d, J=8.0 Hz, 1H), 7.12 (d, J=8.5 Hz, 1H), 6.47 (d, J=1.0 Hz, 1H), 6.03 (dd, J=7.3, 1.8 Hz, 1H), 4.57 (s, 2H), 3.84 (s, 2H), 3.10 (t, J=7.8 Hz, 2H), 2.95 (t, J=8.0 Hz, 2H), 2.87 (s, 2H), 1.51 (s, 9H); ESI MS m/z 506 [M+H]⁺.

### f) 4-(2-(5-Chloropyridin-2-yl)ethyl)-1-(1,2,3,4-tetrahydrobenzofuro[3,2-c]pyridin-7-yl)pyridin-2(1H)-one

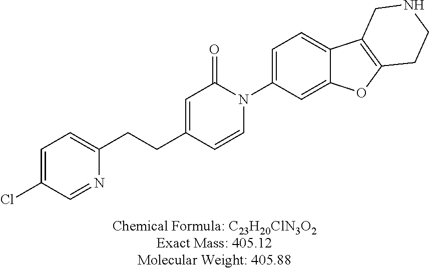 [SMILES: O=c1cc(CCc2ccc(Cl)cn2)ccn1-c1ccc2c3c(oc2c1)CCNC3]

A suspension of tert-butyl 7-(4-(2-(5-chloropyridin-2-yl)ethyl)-2-oxopyridin-1(2H)-yl)-3,4-dihydrobenzofuro[3,2-c]pyridine-2(1H)-carboxylate (188 mg, 0.372 mmol) in MeOH (4 mL) was treated with 2 N HCl in Et₂O (8 mL), and the resulting solution was stirred at ambient temperature for 16 h and then concentrated in vacuo. The resultant HCl salt was converted to the corresponding free base using an SCX-2 cartridge to provide the title compound (155 mg, quant. yield) as an off-white solid: ¹H NMR (500 MHz, CDCl₃) δ 8.52 (d, J=2.5 Hz, 1H), 7.59 (dd, J=8.5, 2.5 Hz, 1H), 7.45-7.43 (m, 2H), 7.29 (d, J=7.0 Hz, 1H), 7.18 (dd, J=8.3, 1.8 Hz, 1H), 7.12 (d, J=8.5 Hz, 1H), 6.48 (s, 1H), 6.11 (dd, J=7.0, 1.5 Hz, 1H), 3.99 (t, J=1.8 Hz, 2H), 3.26 (t, J=5.5 Hz, 2H), 3.10 (t, J=7.8 Hz, 2H), 2.95 (t, J=7.8 Hz, 2H), 2.82-2.80 (m, 2H); ESI MS m/z 406 [M+H]⁺.

### g) 4-(2-(5-Chloropyridin-2-yl)ethyl)-1-(1,2,3,4-tetrahydrobenzofuro[3,2-c]pyridin-7-yl)pyridin-2(1H)-one hydrochloride

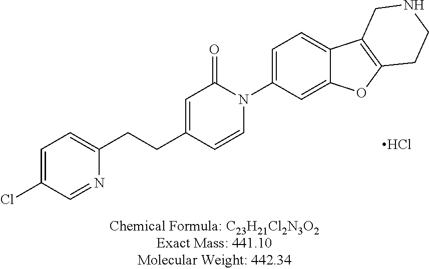 [SMILES: O=c1cc(CCc2ccc(Cl)cn2)ccn1-c1ccc2c3c(oc2c1)CCNC3 ; Cl]

A solution of 4-(2-(5-chloropyridin-2-yl)ethyl)-1-(1,2,3,4-tetrahydrobenzofuro[3,2-c]pyridin-7-yl)pyridin-2(1H)-one (153 mg, 0.377 mmol) in MeOH (5.0 mL) was treated with 2 N HCl in Et₂O (0.19 mL, 0.38 mmol), and the resulting suspension was concentrated. The solid was suspended in H₂O (3 mL), frozen, and lyophilized overnight to provide the title compound (171 mg, quant. yield) as an off-white solid: mp 285-288° C.; ¹H NMR (500 MHz, DMSO-d₆) δ 9.76 (s, 2H), 8.59 (d, J=2.5 Hz, 1H), 7.90 (dd, J=8.3, 2.8 Hz, 1H), 7.70-7.69 (m, 2H), 7.59 (d, J=7.0 Hz, 1H), 7.44 (d, J=8.5 Hz, 1H), 7.28 (dd, J=8.5, 2.0 Hz, 1H), 6.32 (s, 1H), 6.28 (dd, J=7.0, 1.5 Hz, 1H), 4.34 (s, 2H), 3.55-3.52 (m, 2H), 3.13-3.08 (m, 4H), 2.91 (t, J=7.3 Hz, 2H); ESI MS m/z 406 [M+H]⁺; HPLC (Method A)>99% (AUC), t_{R}=12.6 min.

### Example 106

### Preparation of 1-(2,3,4,5-Tetrahydro-1H-benzofuro[3,2-c]azepin-8-yl)-4-(6-(trifluoromethyl)pyridazin-3-yl)pyridin-2(1H)-one hydrochloride

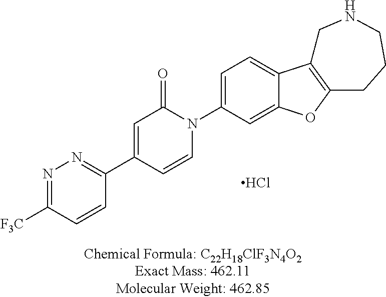 [SMILES: O=c1cc(-c2ccc(C(F)(F)F)nn2)ccn1-c1ccc2c3c(oc2c1)CCCNC3 ; Cl]

1-(2,3,4,5-Tetrahydro-1H-benzofuro[3,2-c]azepin-8-yl)-4-(6-(trifluoromethyl)pyridazin-3-yl)pyridin-2(1H)-one hydrochloride was prepared according to the procedure for Example 36 to provide the title compound as a yellow solid: ¹H NMR (500 MHz, DMSO-d₆) δ 9.32 (s, 2H), 8.71 (d, J=9.0 Hz, 1H), 8.47 (d, J=9.0 Hz, 1H), 7.93 (d, J=7.0 Hz, 1H), 7.83 (d, J=8.5 Hz, 1H), 7.78 (d, J=1.5 Hz, 1H), 7.40-7.38 (m, 2H), 7.19 (dd, J=7.5, 2.0 Hz, 1H), 4.44 (s, 2H), 3.50-3.48 (m, 2H), 3.14-3.12 (m, 2H), 2.11-2.09 (m, 2H); ESI MS m/z 427 [M+H]⁺; HPLC (Method B)>99% (AUC), t_{R}=13.2 min.

### Example 107

### Preparation of 4-(2-(5-Chloropyridin-2-yl)ethyl)-1-(2,3,4,5-tetrahydro-1H-benzofuro[3,2-c]azepin-8-yl)pyridin-2(1H)-one hydrochloride

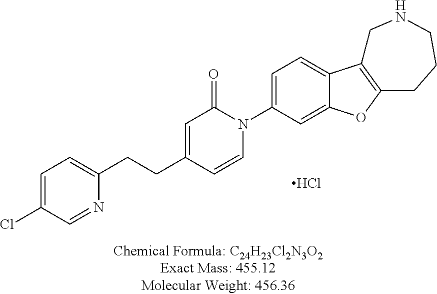 [SMILES: O=c1cc(CCc2ccc(Cl)cn2)ccn1-c1ccc2c3c(oc2c1)CCCNC3 ; Cl]

4-(2-(5-Chloropyridin-2-yl)ethyl)-1-(2,3,4,5-tetrahydro-1H-benzofuro[3,2-c]azepin-8-yl)pyridin-2(1H)-one hydrochloride was prepared according to the procedure for Example 40 to provide the title compound as a white solid: ¹H NMR (500 MHz, DMSO-d₆) δ 9.29 (s, 2H), 8.57-8.56 (m, 1H), 7.88-7.86 (m, 1H), 7.77 (dd, J=8.5, 1.5 Hz, 1H), 7.64-7.63 (m, 1H), 7.58 (dd, J=7.0, 1.5 Hz, 1H), 7.41 (dd, J=8.0, 1.0 Hz, 1H), 7.28-7.25 (m, 1H), 6.31-6.27 (m, 2H), 4.41 (br s, 2H), 3.47 (br s, 2H), 3.12-3.07 (m, 4H), 2.92-2.89 (m, 2H), 2.11-2.08 (m, 2H); ESI MS m/z 420 [M+H]⁺; HPLC (Method B)>99% (AUC), t_{R}=12.9 min.

### Example 108

### Preparation of 4-Phenethyl-1-(2,3,4,5-tetrahydro-1H-benzofuro[3,2-c]azepin-8-yl)pyridin-2(1H)-one hydrochloride

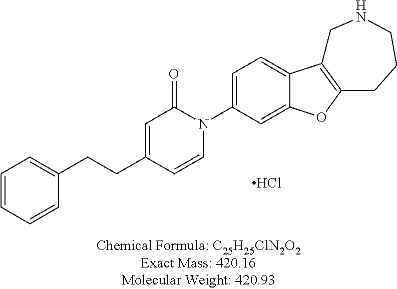 [SMILES: O=c1cc(CCc2ccccc2)ccn1-c1ccc2c3c(oc2c1)CCCNC3 ; Cl]

4-Phenethyl-1-(2,3,4,5-tetrahydro-1H-benzofuro[3,2-c]azepin-8-yl)pyridin-2(1H)-one hydrochloride was prepared according to the procedure for Example 40 to provide the title compound as a white solid: ¹H NMR (500 MHz, DMSO-d₆) δ 9.43 (s, 2H), 7.77 (d, J=8.5 Hz, 1H), 7.64 (d, J=2.0 Hz, 1H), 7.59 (d, J=7.0 Hz, 1H), 7.33-7.26 (m, 5H), 7.22-7.19 (m, 1H), 6.32-6.30 (m, 2H), 4.40 (s, 2H), 3.48-3.46 (m, 2H), 3.12-3.10 (m, 2H), 2.93-2.90 (m, 2H), 2.80-2.77 (m, 2H), 2.11-2.07 (m, 2H); ESI MS m/z 385 [M+H]⁺; HPLC (Method B)>99% (AUC), t_{R}=14.3 min.

### Example 109

### Preparation of 4-((5-Fluoropyridin-2-yl)methoxy)-1-(2,3,4,5-tetrahydro-1H-benzofuro[2,3-c]azepin-7-yl)pyridin-2(1H)-one hydrochloride

### a) tert-Butyl 7-(4-((5-fluoropyridin-2-yl)methoxy)-2-oxopyridin-1(2H)-yl)-4,5-dihydro-1H-benzofuro[2,3-c]azepine-2(3H)-carboxylate

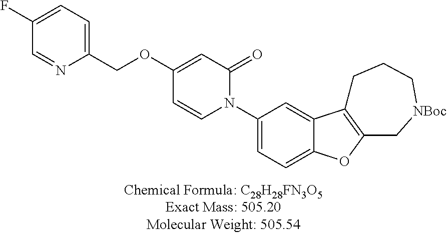 [SMILES: CC(C)(C)OC(=O)N1CCCc2c(oc3ccc(-n4ccc(OCc5ccc(F)cn5)cc4=O)cc23)C1]

tert-Butyl 7-bromo-4,5-dihydro-1H-benzofuro[2,3-c]azepine-2(3H)-carboxylate (110 mg, 0.30 mmol) and 4-((5-fluoropyridin-2-yl)methoxy)pyridin-2(1H)-one (66 mg, 0.30 mmol) were reacted according to Example 7 (step a) to provide the title compound (950 mg, 62%) as an off-white solid: ¹H NMR (300 MHz, CDCl₃) δ 8.48 (d, J=2.1 Hz, 1H), 7.54-7.41 (m, 3H), 7.39 (d, J=1.8 Hz, 1H), 7.29 (d, J=7.5 Hz, 1H), 7.22-7.10 (m, 1H), 6.12-6.03 (m, 2H), 5.16 (s, 2H), 4.79-4.62 (m, 2H), 3.74-3.57 (m, 2H), 2.74 (t, J=6.0 Hz, 2H), 2.08-1.94 (m, 2H), 1.50-1.38 (m, 9H); APCI MS m/z 506 [M+H]⁺.

### b) 4-((5-Fluoropyridin-2-yl)methoxy)-1-(2,3,4,5-tetrahydro-1H-benzofuro[2,3-c]azepin-7-yl)pyridin-2(1H)-one hydrochloride

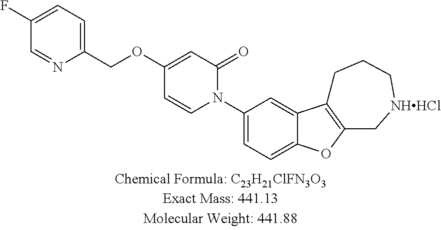

tert-Butyl 7-(4-((5-fluoropyridin-2-yl)methoxy)-2-oxopyridin-1(2H)-yl)-4,5-dihydro-1H-benzofuro[2,3-c]azepine-2(3H)-carboxylate (95 mg, 0.18 mmol) and conc. HCl (0.6 mL) were reacted according to Example 74 (step b) to provide the title compound (80 mg, 96%) as an off white solid: ¹H NMR (400 MHz, DMSO-d₆) δ 9.89-9.80 (m, 2H), 8.62 (d, J=3.2 Hz, 1H), 7.88-7.79 (m, 1H), 7.70-7.60 (m, 4H), 7.28 (dd, J=8.8, 2.0 Hz, 1H), 6.15 (dd, J=7.6, 2.4 Hz, 1H), 6.00 (d, J=2.8 Hz, 1H), 5.22 (s, 2H), 4.55-4.46 (m, 2H), 3.51-3.41 (m, 2H), 2.85 (t, J=5.6 Hz, 2H), 2.14-2.03 (m, 2H); APCI MS m/z 406 [M+H]⁺; HPLC (Method C)>99% (AUC), t_{R}=19.44 min.

### Example 110

### Preparation of 4-(Benzyloxy)-1-(2,3,4,5-tetrahydro-1H-benzofuro[2,3-c]azepin-7-yl)pyridin-2(1H)-one hydrochloride

### a) tert-butyl 7-bromo-4,5-dihydro-1H-benzofuro[2,3-c]azepine-2(3H)-carboxylate

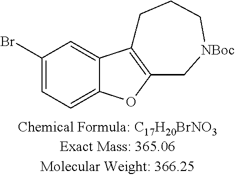 [SMILES: CC(C)(C)OC(=O)N1CCCc2c(oc3ccc(Br)cc23)C1]

O-(4-Bromophenyl)hydroxylamine hydrochloride (1.0 g, 4.5 mmol) and tert-butyl 3-oxoazepane-1-carboxylate (1.14 g, 5.34 mmol) were reacted according to Example 2 (step b) to provide the title compound (220 mg, 14%) as an off-white solid ¹H NMR (400 MHz, CDCl₃) δ 7.53 (br s, 1H), 7.36-7.22 (m, 2H), 4.76-4.60 (m, 2H), 3.72-3.56 (m, 2H), 2.72 (t, J=6.0 Hz, 2H), 2.09-1.95 (m, 2H), 1.50-1.38 (m, 9H).

### b) tert-Butyl 7-(4-(benzyloxy)-2-oxopyridin-1(2H)-yl)-4,5-dihydro-1H-benzofuro[2,3-c]azepine-2(3H)-carboxylate

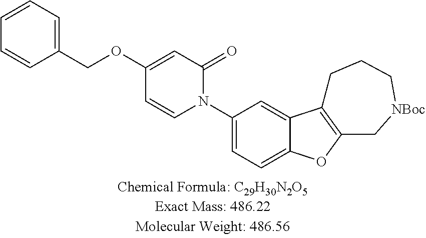 [SMILES: CC(C)(C)OC(=O)N1CCCc2c(oc3ccc(-n4ccc(OCc5ccccc5)cc4=O)cc23)C1]

tert-Butyl 7-bromo-4,5-dihydro-1H-benzofuro[2,3-c]azepine-2(3H)-carboxylate (110 mg, 0.30 mmol) and 4-benzyloxy pyridinone (62 mg, 0.30 mmol) were reacted according to Example 7 (step a) to provide the title compound (120 mg, 82%) as an off-white solid: ¹H NMR (300 MHz, CDCl₃) δ 7.50 (m, 7H), 7.27 (d, J=7.5 Hz, 1H), 7.21-7.11 (m, 1H), 6.10-6.01 (m, 2H), 5.04 (s, 2H), 4.79-4.62 (m, 2H), 3.74-3.57 (m, 2H), 2.74 (t, J=5.7 Hz, 2H), 2.08-1.94 (m, 2H), 1.50-1.38 (m, 9H); APCI MS m/z 487 [M+H]⁺.

### c) 4-(Benzyloxy)-1-(2,3,4,5-tetrahydro-1H-benzofuro[2,3-c]azepin-7-yl)pyridin-2(1H)-one hydrochloride

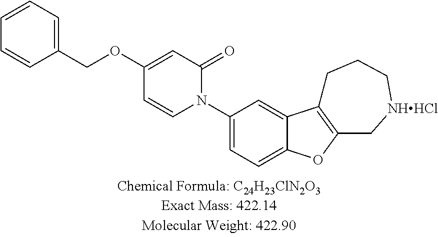

tert-Butyl 7-(4-(benzyloxy)-2-oxopyridin-1(2H)-yl)-4,5-dihydro-1H-benzofuro[2,3-c]azepine-2(3H)-carboxylate (120 mg, 0.24 mmol) and cone. HCl (0.6 mL) were reacted according to Example 74 (step b) to provide the title compound (100 mg, 96%) as a white solid: ¹H NMR (400 MHz, DMSO-d₆) δ 9.69-9.58 (m, 2H), 7.68-7.58 (m, 3H), 7.50-7.34 (m, 5H), 7.28 (dd, J=8.8, 1H), 6.12 (dd, J=7.6, 1H), 5.99 (d, J=2.8 Hz, 1H), 5.15 (s, 2H), 4.56-4.49 (m, 2H), 3.52-3.42 (m, 2H), 2.85 (t, J=5.2 Hz, 2H), 2.13-2.02 (m, 2H); APCI MS m/z 387 [M+H]⁺; HPLC (Method C)>99% (AUC), t_{R}=20.77 min.

### Example 111

### Preparation of 4-(2-Fluoro-4-methoxyphenyl)-1-(1,2,3,4-tetrahydrobenzofuro[3,2-c]pyridin-7-yl)pyridin-2(1H)-one hydrochloride

### a) 4-(2-Fluoro-4-methoxyphenyl)pyridin-2(1H)-one

CAS Registry Number 1173157-92-0

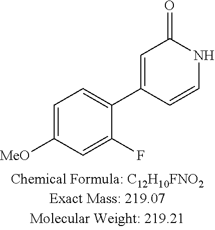 [SMILES: COc1ccc(-c2cc[nH]c(=O)c2)c(F)c1]

This compound was prepared in accordance with the procedure described in PCT Publication No. WO 2009/089482 to Guzzo et al., which is hereby incorporated by reference in its entirety.

### b) tert-Butyl 7-(4-(2-fluoro-4-methoxyphenyl)-2-oxopyridin-1(2H)-yl)-3,4-dihydrobenzofuro[3,2-c]pyridine-2(1H)-carboxylate

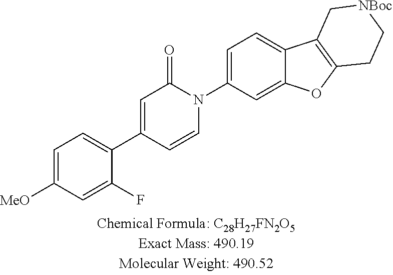 [SMILES: COc1ccc(-c2ccn(-c3ccc4c5c(oc4c3)CCN(C(=O)OC(C)(C)C)C5)c(=O)c2)c(F)c1]

tert-Butyl 7-bromo-3,4-dihydrobenzofuro[3,2-c]pyridine-2(1H)-carboxylate (200 mg, 0.568 mmol) and 4-(2-fluoro-4-methoxyphenyl)pyridin-2(1H)-one (125 mg, 0.570 mmol) were reacted according to Example 3 (step b) to provide the title compound (244 mg, 87%) as a white solid: ¹H NMR (500 MHz, CDCl₃) δ 7.54 (d, J=2.0 Hz, 1H), 7.51 (d, J=8.0 Hz, 1H), 7.47-7.39 (m, 2H), 7.28 (br s, 1H), 6.82 (d, J=1.5 Hz, 1H), 6.81 (dd, J=8.5, 2.5 Hz, 1H), 6.73 (dd, J=12.5, 2.5 Hz, 1H), 6.51 (dt, J=7.0, 2.0 Hz, 1H), 4.58 (s, 2H), 3.86 (s, 3H), 3.85 (s, 2H), 2.89 (s, 2H), 1.52 (s, 9H); ESI MS m/z 491 [M+H]⁺.

### c) 4-(2-Fluoro-4-methoxyphenyl)-1-(1,2,3,4-tetrahydrobenzofuro[3,2-c]pyridin-7-yl)pyridin-2(1H)-one

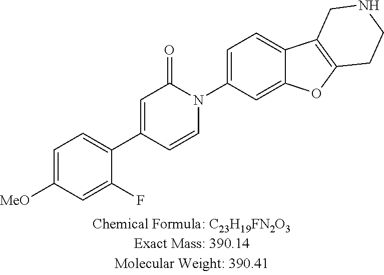 [SMILES: COc1ccc(-c2ccn(-c3ccc4c5c(oc4c3)CCNC5)c(=O)c2)c(F)c1]

A solution of tert-butyl 7-(4-(2-fluoro-4-methoxyphenyl)-2-oxopyridin-1(2H)-yl)-3,4-dihydrobenzofuro[3,2-c]pyridine-2(1H)-carboxylate (243 mg, 0.495 mmol) in MeOH (5 mL) was treated with 2 N HCl in Et₂O (10 mL), and the resulting solution was stirred at ambient temperature for 16 h and then concentrated in vacuo. The resultant HCl salt was converted to the corresponding free base using an SCX-2 cartridge to provide the title compound (201 mg, quant. yield) as a light yellow solid: ¹H NMR (500 MHz, CDCl₃) δ 7.52 (d, J=1.5 Hz, 1H), 7.48-7.40 (m, 3H), 7.24 (d, J=2.0 Hz, 1H), 6.82 (d, J=1.5 Hz, 1H), 6.80 (dd, J=8.5, 2.5 Hz, 1H), 6.73 (dd, J=12.5, 2.5 Hz, 1H), 6.50 (dt, J=7.5, 2.0 Hz, 1H), 4.01 (t, J=1.8 Hz, 2H), 3.86 (s, 3H), 3.26 (t, J=5.8 Hz, 2H), 2.83-2.81 (m, 2H); ESI MS m/z 391 [M+H]⁺.

### d) 4-(2-Fluoro-4-methoxyphenyl)-1-(1,2,3,4-tetrahydrobenzofuro[3,2-c]pyridin-7-yl)pyridin-2(1H)-one hydrochloride

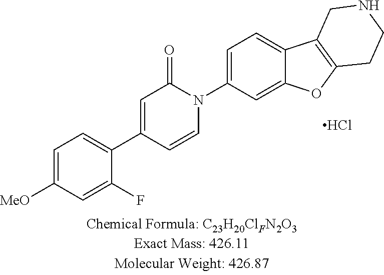 [SMILES: COc1ccc(-c2ccn(-c3ccc4c5c(oc4c3)CCNC5)c(=O)c2)c(F)c1 ; Cl]

A solution of 4-(2-fluoro-4-methoxyphenyl)-1-(1,2,3,4-tetrahydrobenzofuro[3,2-c]pyridin-7-yl)pyridin-2(1H)-one (199 mg, 0.510 mmol) in MeOH (5 mL) was treated with 2 N HCl in Et₂O (0.26 mL, 0.51 mmol), and the resulting suspension was concentrated. The solid was suspended in H₂O (3 mL), frozen, and lyophilized overnight to provide the title compound (207 mg, 95%) as an off-white solid: mp 282-284° C.; ¹H NMR (500 MHz, DMSO-d₆) δ 9.63 (s, 2H), 7.79 (d, J=1.5 Hz, 1H), 7.75-7.72 (m, 2H), 7.61 (t, J=8.8 Hz, 1H), 7.37 (dd, J=8.0, 1.5 Hz, 1H), 7.01 (dd, J=13.3, 2.3 Hz, 1H), 6.93 (dd, J=8.8, 2.3 Hz, 1H), 6.63 (s, 1H), 6.55-6.54 (m, 1H), 4.37 (s, 2H), 3.84 (s, 3H), 3.57-3.54 (m, 2H), 3.14-3.12 (m, 2H); ESI MS m/z 391 [M+H]⁺; HPLC (Method A)>99% (AUC), t_{R}=13.6 min.

### Example 112

### Preparation of 4-(2-Chloro-4-methoxyphenyl)-1-(1,2,3,4-tetrahydrobenzofuro[3,2-c]pyridin-7-yl)pyridin-2(1H)-one hydrochloride

### a) 4-(2-Chloro-4-methoxyphenyl)-2-methoxypyridine

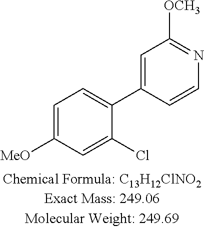 [SMILES: COc1ccc(-c2ccnc(OC)c2)c(Cl)c1]

A mixture of 4-bromo-2-methoxypyridine (1.20 g, 6.38 mmol), 2-chloro-4-methoxyphenylboronic acid (2.38 g, 12.8 mmol), and potassium carbonate (2.65 g, 19.1 mmol) in anhydrous DMSO (12 mL) was degassed for several minutes with argon. Dichloro[1,1′-bis(diphenylphosphino)ferrocene]palladium(II) dichloromethane adduct (261 mg, 0.32 mmol) was added and, after degassing briefly with argon again, the flask was sealed, and the reaction was heated to 90° C. and stirred for 2 h. After cooling to rt, the reaction mixture was diluted with ethyl acetate, washed with 5% lithium chloride solution (4×), dried over sodium sulfate, filtered, and concentrated. Flash chromatography (80 g ISCO Gold column, 2%-20% ethyl acetate/hexanes) provided the title compound (1.51 g, 95%) as a white solid: ¹H NMR (500 MHz, CDCl₃) δ 8.19 (d, J=5.0 Hz, 1H), 7.24 (d, J=8.5 Hz, 1H), 7.02 (d, J=2.0 Hz, 1H), 6.96 (d, J=5.0 Hz, 1H), 6.88 (dd, J=8.3, 2.3 Hz, 1H), 6.08 (s, 1H), 3.98 (s, 3H), 3.84 (s, 3H); ESI MS m/z 250 [M+H]⁺.

### b) 4-(2-Chloro-4-methoxyphenyl)pyridin-2(1H)-one

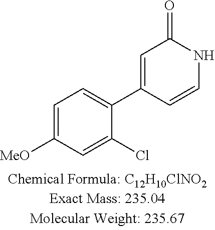 [SMILES: COc1ccc(-c2cc[nH]c(=O)c2)c(Cl)c1]

A mixture of 4-(2-chloro-4-methoxyphenyl)-2-methoxypyridine (1.50 g, 6.01 mmol) and c. HCl (50 mL) was heated to 120° C. and stirred for 24 h. A mixture of products was obtained resulting from both mono- and bis-demethylation. After cooling to rt, the mixture was concentrated in vacuo. The resultant solid was dissolved in methanol and the pH was adjusted to 8 using 6 N NaOH. The methanol was removed, additional water was added, and the mixture was cooled in an ice bath. The solid precipitate was filtered and rinsed with cold water, providing 1.29 g of a white solid (still a mixture of the two products). Due to poor solubility, only a portion of the material was purified further. Flash chromatography (120 g ISCO Gold column, 12%-100% B; A=CH₂Cl₂, B=80:18:2 CH₂Cl₂/MeOH/NH₄OH) provided the title compound (177 mg) as a white solid: ¹H NMR (500 MHz, CDCl₃) δ 12.26 (s, 1H), 7.36 (d, J=7.0 Hz, 1H), 7.26 (d, J=8.5 Hz, 1H), 7.02 (d, J=2.5 Hz, 1H), 6.88 (dd, J=8.5, 2.5 Hz, 1H), 6.61 (d, J=1.0 Hz, 1H), 6.41 (dd, J=6.8, 1.8 Hz, 1H), 3.85 (s, 3H); ESI MS m/z 236 [M+H]⁺.

### c) tert-Butyl 7-(4-(2-chloro-4-methoxyphenyl)-2-oxopyridin-1(2H)-yl)-3,4-dihydrobenzofuro[3,2-c]pyridine-2(1H)-carboxylate

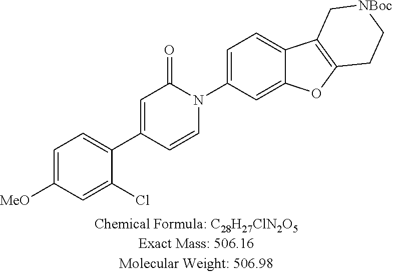 [SMILES: COc1ccc(-c2ccn(-c3ccc4c5c(oc4c3)CCN(C(=O)OC(C)(C)C)C5)c(=O)c2)c(Cl)c1]

tert-Butyl 7-bromo-3,4-dihydrobenzofuro[3,2-c]pyridine-2(1H)-carboxylate (120 mg, 0.341 mmol) and 4-(2-chloro-4-methoxyphenyl)pyridin-2(1H)-one (80 mg, 0.34 mmol) were reacted according to Example 3 (step b) to provide the title compound (171 mg, 99%) as a colorless semi-solid: ¹H NMR (500 MHz, CDCl₃) δ 7.55 (d, J=1.5 Hz, 1H), 7.52 (d, J=8.5 Hz, 1H), 7.39 (d, J=7.0 Hz, 1H), 7.31 (d, J=8.5 Hz, 1H), 7.29 (br s, 1H), 7.03 (d, J=2.5 Hz, 1H), 6.90 (dd, J=8.5, 2.5 Hz, 1H), 6.70 (d, J=1.5 Hz, 1H), 6.41 (dd, J=7.3, 1.8 Hz, 1H), 4.58 (s, 2H), 3.87 (s, 3H), 3.86 (s, 2H), 2.89 (s, 2H), 1.52 (s, 9H); ESI MS m/z 507 [M+H]⁺.

### d) 4-(2-Chloro-4-methoxyphenyl)-1-(1,2,3,4-tetrahydrobenzofuro[3,2-c]pyridin-7-yl)pyridin-2(1H)-one

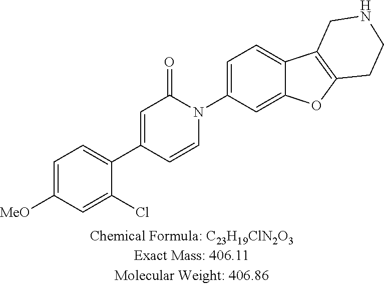 [SMILES: COc1ccc(-c2ccn(-c3ccc4c5c(oc4c3)CCNC5)c(=O)c2)c(Cl)c1]

A solution of tert-butyl 7-(4-(2-chloro-4-methoxyphenyl)-2-oxopyridin-1(2H)-yl)-3,4-dihydrobenzofuro[3,2-c]pyridine-2(1H)-carboxylate (169 mg, 0.333 mmol) in MeOH (4 mL) was treated with 2 N HCl in Et₂O (8 mL), and the resulting solution was stirred at ambient temperature for 16 h and then concentrated in vacuo. The resultant HCl salt was converted to the corresponding free base using an SCX-2 cartridge to provide the title compound (126 mg, 94%) as a light yellow solid: ¹H NMR (500 MHz, CDCl₃) δ 7.54 (d, J=1.5 Hz, 1H), 7.47 (d, J=8.5 Hz, 1H), 7.40 (d, J=7.0 Hz, 1H), 7.31 (d, J=8.5 Hz, 1H), 7.28 (d, J=2.0 Hz, 1H), 7.03 (d, J=2.5 Hz, 1H), 6.90 (dd, J=8.5, 2.5 Hz, 1H), 6.70 (d, J=2.0 Hz, 1H), 6.41 (dd, J=7.0, 2.0 Hz, 1H), 4.01 (t, J=1.8 Hz, 2H), 3.86 (s, 3H), 3.27 (t, J=5.8 Hz, 2H), 2.84-2.82 (m, 2H); ESI MS m/z 407 [M+H]⁺.

### e) 4-(2-Chloro-4-methoxyphenyl)-1-(1,2,3,4-tetrahydrobenzofuro[3,2-c]pyridin-7-yl)pyridin-2(1H)-one hydrochloride

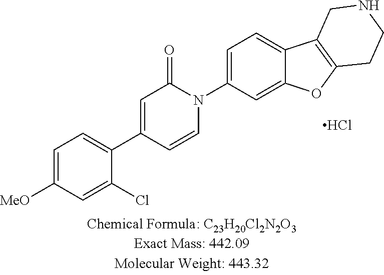 [SMILES: COc1ccc(-c2ccn(-c3ccc4c5c(oc4c3)CCNC5)c(=O)c2)c(Cl)c1 ; Cl]

A solution of 4-(2-chloro-4-methoxyphenyl)-1-(1,2,3,4-tetrahydrobenzofuro[3,2-c]pyridin-7-yl)pyridin-2(1H)-one (123 mg, 0.302 mmol) in MeOH (5 mL) was treated with 2 N HCl in Et₂O (0.15 mL, 0.30 mmol), and the resulting suspension was concentrated. The solid was suspended in H₂O (3 mL), frozen, and lyophilized overnight to provide the title compound (133 mg, 91%) as a white solid: mp 294-297° C.; ¹H NMR (500 MHz, DMSO-d₆) δ 9.56 (s, 2H), 7.81 (d, J=1.5 Hz, 1H), 7.75-7.73 (m, 2H), 7.45 (d, J=8.5 Hz, 1H), 7.38 (dd, J=8.3, 1.8 Hz, 1H), 7.20 (d, J=2.5 Hz, 1H), 7.06 (dd, J=8.5, 2.5 Hz, 1H), 6.50 (d, J=1.5 Hz, 1H), 6.43 (dd, J=7.3, 1.8 Hz, 1H), 4.37 (s, 2H), 3.84 (s, 3H), 3.56 (t, J=5.8 Hz, 2H), 3.13 (t, J=5.8 Hz, 2H); ESI MS m/z 407 [M+H]⁺; HPLC (Method A)>99% (AUC), t_{R}=14.0 min.

### Example 113

### Preparation of 1-(1,2,3,4-Tetrahydrobenzofuro[3,2-c]pyridin-7-yl)-4-((6-(trifluoromethyl)pyridazin-3-yl)methoxy)pyridin-2(1H)-one hydrochloride

### a) 3-Methyl-6-(trifluoromethyl)pyridazine

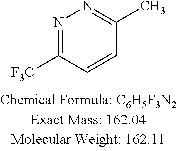

A solution of 3-chloro-6-(trifluoromethyl)pyridazine (1.00 g, 5.48 mmol) in 1,4-dioxane (30 mL) was degassed with a stream of nitrogen for 15 min. Tetrakis(triphenylphosphine)palladium(0) (317 mg, 0.27 mmol) was added followed by trimethylaluminum (2 M in toluene, 5.5 mL, 11 mmol). The reaction was heated to reflux under nitrogen and stirred for 4 h. After cooling to rt, the reaction was quenched with methanol and then concentrated in vacuo. The resulting material was partitioned between ethyl acetate and water and then filtered through sharkskin filter paper to remove the precipitate that formed. The two layers of the filtrate were separated and the aqueous phase was extracted with additional ethyl acetate. The combined organic extracts were washed with brine, dried over sodium sulfate, filtered, and concentrated. Flash chromatography (40 g ISCO Gold column, 12%-100% ethyl acetate/hexanes) provided the title compound (644 mg, 72%) as a light yellow solid: ¹H NMR (500 MHz, CDCl₃) δ 7.71 (d, J=8.5 Hz, 1H), 7.51 (d, J=8.5 Hz, 1H), 2.84 (s, 3H); ESI MS m/z 163 [M+H]⁺.

### b) 3-(Bromomethyl)-6-(trifluoromethyl)pyridazine

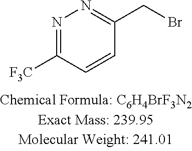 [SMILES: FC(F)(F)c1ccc(CBr)nn1]

A mixture of N-bromosuccinimide (748 mg, 4.16 mmol), 2,2′-azobis-(2-methylpropionitrile) (100 mg, 0.59 mmol), and 3-methyl-6-(trifluoromethyl)pyridazine (642 mg, 3.96 mmol) in carbon tetrachloride (20 mL) was heated to reflux and stirred for 28 h. A mixture of starting material, desired product, and dibrominated product was obtained. After cooling to rt, the reaction mixture was concentrated in vacuo. Flash chromatography (40 g ISCO Gold column, 7%-60% ethyl acetate/hexanes) provided the title compound (264 mg, 28%) as a red oil: ¹H NMR (500 MHz, CDCl₃) δ 7.88 (d, J=9.0 Hz, 1H), 7.85 (d, J=9.0 Hz, 1H), 4.82 (s, 2H); ESI MS m/z 242 [M+H]⁺.

### c) 3-(((2-Chloropyridin-4-yl)oxy)methyl)-6-(trifluoromethyl)pyridazine

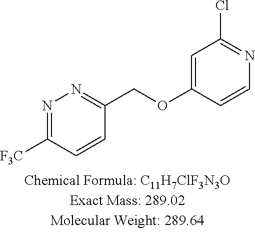 [SMILES: FC(F)(F)c1ccc(COc2ccnc(Cl)c2)nn1]

A mixture of 2-chloro-4-hydroxypyridine (376 mg, 2.90 mmol) and potassium carbonate (802 mg, 5.80 mmol) in anhydrous DMF (5 mL) was stirred at rt for 2 h. A solution of 3-(bromomethyl)-6-(trifluoromethyl)pyridazine (699 mg, 2.90 mmol) in DMF (15 mL) was added dropwise and the reaction was heated to 60° C. and stirred for 16 h. After cooling to rt, the mixture was poured over ice water and extracted with ethyl acetate (2×). The combined organic extracts were washed with 5% lithium chloride solution (3×), dried over sodium sulfate, filtered, and concentrated. Flash chromatography (80 g ISCO Gold column, 12%-100% ethyl acetate/hexanes) provided the title compound (543 mg, 65%) as a yellow solid: ¹H NMR (500 MHz, CDCl₃) δ 8.28 (d, J=6.0 Hz, 1H), 7.93-7.89 (m, 2H), 7.00 (d, J=2.5 Hz, 1H), 6.89 (dd, J=5.8, 2.3 Hz, 1H), 5.58 (s, 2H); ESI MS m/z 290 [M+H]⁺.

### d) 4-((6-(Trifluoromethyl)pyridazin-3-yl)methoxy)pyridin-2(1H)-one

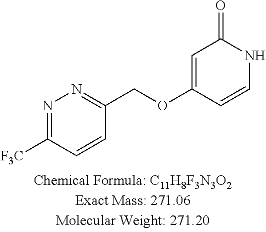 [SMILES: O=c1cc(OCc2ccc(C(F)(F)F)nn2)cc[nH]1]

A mixture of 3-(((2-chloropyridin-4-yl)oxy)methyl)-6-(trifluoromethyl)pyridazine (740 mg, 2.55 mmol) and ammonium acetate (2.03 g, 25.6 mmol) in 1:1 formic acid/water (10 mL) was heated to 110° C. and stirred for 72 h. After cooling to rt, the mixture was concentrated in vacuo, cooled in an ice bath, and the pH adjusted to 8 using 6 N NaOH. The resultant solid was filtered, rinsed with a minimal amount of cold water, and then dried in a vacuum oven at 55° C. overnight, providing the title compound (588 mg, 80%) as a brown solid: ¹H NMR (500 MHz, DMSO-d₆) δ 11.19 (br s, 1H), 8.34 (d, J=9.0 Hz, 1H), 8.13 (d, J=9.0 Hz, 1H), 7.30 (d, J=7.5 Hz, 1H), 5.99 (dd, J=7.2, 2.5 Hz, 1H), 5.86 (d, J=2.5 Hz, 1H), 5.52 (s, 2H); ESI MS m/z 272 [M+H]⁺.

### e) tert-Butyl 7-(2-oxo-4-((6-(trifluoromethyl)pyridazin-3-yl)methoxy)pyridin-1(2H)-yl)-3,4-dihydrobenzofuro[3,2-c]pyridine-2(1H)-carboxylate

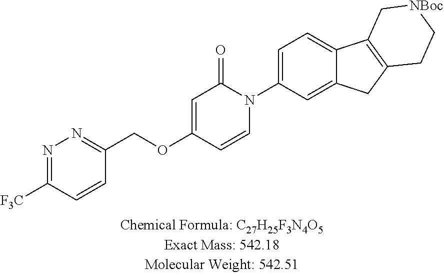 [SMILES: CC(C)(C)OC(=O)N1CCC2=C(C1)c1ccc(-n3ccc(OCc4ccc(C(F)(F)F)nn4)cc3=O)cc1C2]

tert-Butyl 7-bromo-3,4-dihydrobenzofuro[3,2-c]pyridine-2(1H)-carboxylate (200 mg, 0.568 mmol) and 4-((6-(trifluoromethyl)pyridazin-3-yl)methoxy)pyridin-2(1H)-one (164 mg, 0.605 mmol) were reacted according to Example 3 (step b) to provide the title compound (234 mg, 76%) as an off-white solid: ¹H NMR (500 MHz, CDCl₃) δ 7.92-7.88 (m, 2H), 7.49 (d, J=8.0 Hz, 1H), 7.46 (d, J=2.0 Hz, 1H), 7.34 (d, J=7.5 Hz, 1H), 7.20 (d, J=7.5 Hz, 1H), 6.12 (dd, J=7.5, 2.5 Hz, 1H), 6.09 (d, J=2.5 Hz, 1H), 5.53 (s, 2H), 4.57 (s, 2H), 3.84 (s, 2H), 2.88 (s, 2H), 1.51 (s, 9H); ESI MS m/z 543 [M+H]⁺.

### f) 1-(1,2,3,4-Tetrahydrobenzofuro[3,2-c]pyridin-7-yl)-4-((6-(trifluoromethyl)pyridazin-3-yl)methoxy)pyridin-2(1H)-one

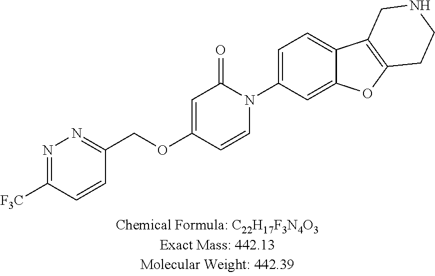 [SMILES: O=c1cc(OCc2ccc(C(F)(F)F)nn2)ccn1-c1ccc2c3c(oc2c1)CCNC3]

A suspension of tert-butyl 7-(2-oxo-4-((6-(trifluoromethyl)pyridazin-3-yl)methoxy)pyridin-1(2H)-yl)-3,4-dihydrobenzofuro[3,2-c]pyridine-2(1H)-carboxylate (231 mg, 0.426 mmol) in MeOH (4.5 mL) was treated with 2 N HCl in Et₂O (9 mL), and the resulting solution was stirred at ambient temperature for 16 h and then concentrated in vacuo. The resultant HCl salt was converted to the corresponding free base using an SCX-2 cartridge to provide the title compound (195 mg, quant. yield) as a light yellow solid: ¹H NMR (500 MHz, CDCl₃) δ 7.92-7.88 (m, 2H), 7.47-7.44 (m, 2H), 7.35 (d, J=7.5 Hz, 1H), 7.17 (dd, J=8.3, 1.8 Hz, 1H), 6.12 (d, J=2.5 Hz, 1H), 6.11-6.09 (m, 1H), 5.53 (s, 2H), 4.00 (s, 2H), 3.26 (t, J=5.8 Hz, 2H), 2.81 (t, J=5.5 Hz, 2H); ESI MS m/z 443 [M+H]⁺.

### g) 1-(1,2,3,4-Tetrahydrobenzofuro[3,2-c]pyridin-7-yl)-4-((6-(trifluoromethyl)pyridazin-3-yl)methoxy)pyridin-2(1H)-one hydrochloride

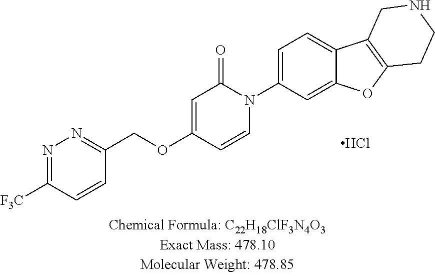 [SMILES: O=c1cc(OCc2ccc(C(F)(F)F)nn2)ccn1-c1ccc2c3c(oc2c1)CCNC3 ; Cl]

A suspension of 1-(1,2,3,4-tetrahydrobenzofuro[3,2-c]pyridin-7-yl)-4-((6-(trifluoromethyl)pyridazin-3-yl)methoxy)pyridin-2(1H)-one (190 mg, 0.429 mmol) in MeOH (5 mL) was treated with 2 N HCl in Et₂O (0.22 mL, 0.43 mmol), and the mixture was concentrated. The resultant solid was suspended in H₂O (3 mL), frozen, and lyophilized overnight to provide the title compound (199 mg, 97%) as a white solid: mp 289-291° C.; ¹H NMR (500 MHz, DMSO-d₆) δ 9.56 (s, 2H), 8.38 (d, J=9.0 Hz, 1H), 8.18 (d, J=9.0 Hz, 1H), 7.71-7.69 (m, 2H), 7.66 (d, J=7.5 Hz, 1H), 7.28 (dd, J=8.3, 1.5 Hz, 1H), 6.22 (dd, J=7.8, 2.8 Hz, 1H), 6.08 (d, J=2.5 Hz, 1H), 5.62 (s, 2H), 4.35 (s, 2H), 3.55 (t, J=6.0 Hz, 2H), 3.12 (t, J=5.8 Hz, 2H); ESI MS m/z 443 [M+H]⁺; HPLC (Method A)>99% (AUC), t_{R}=12.8 min.

### Example 114

### Preparation of 4-(2-Methyl-4-(trifluoromethoxy)phenyl)-1-(1,2,3,4-tetrahydrobenzofuro[3,2-c]pyridin-7-yl)pyridin-2(1H)-one hydrochloride

### a) 2-Methoxy-4-(2-methyl-4-(trifluoromethoxy)phenyl)pyridine

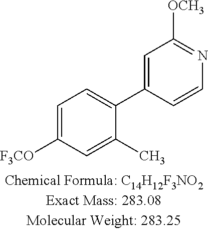 [SMILES: COc1cc(-c2ccc(OC(F)(F)F)cc2C)ccn1]

A mixture of 4-bromo-2-methoxypyridine (658 mg, 3.50 mmol), 2-methyl-4-trifluoromethoxyphenylboronic acid (1.00 g, 4.55 mmol), and potassium carbonate (1.45 g, 10.5 mmol) in anhydrous DMSO (7 mL) was degassed for several minutes with argon. Dichloro[1,1′-bis(diphenylphosphino)ferrocene]palladium(II) dichloromethane adduct (147 mg, 0.18 mmol) was added and, after degassing briefly with argon again, the flask was sealed, and the reaction was heated to 90° C. and stirred for 4 h. After cooling to rt, the reaction mixture was diluted with ethyl acetate, washed with 5% lithium chloride solution (4×), dried over sodium sulfate, filtered, and concentrated. Flash chromatography (40 g ISCO Gold column, 2%-20% ethyl acetate/hexanes) provided the title compound (816 mg, 82%) as a colorless oil: ¹H NMR (500 MHz, CDCl₃) δ 8.20 (d, J=5.0 Hz, 1H), 7.21 (d, J=8.0 Hz, 1H), 7.13 (s, 1H), 7.11 (d, J=8.5 Hz, 1H), 6.09 (dd, J=5.5, 1.5 Hz, 1H), 6.67 (s, 1H), 3.99 (s, 3H), 2.28 (s, 3H); ESI MS m/z 284 [M+H]⁺.

### b) 4-(2-Methyl-4-(trifluoromethoxy)phenyl)pyridin-2(1H)-one

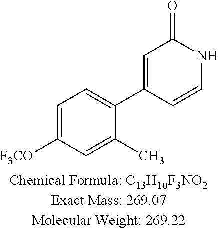 [SMILES: Cc1cc(OC(F)(F)F)ccc1-c1cc[nH]c(=O)c1]

A mixture of 2-methoxy-4-(2-methyl-4-(trifluoromethoxy)phenyl)pyridine (812 mg, 2.87 mmol) and c. HCl (35 mL) was heated to 120° C. and stirred for 16 h. After cooling to rt, the mixture was concentrated in vacuo. The resultant solid was dissolved in methanol and the pH was adjusted to 8 using 6 N NaOH. The methanol was removed, additional water was added, and the mixture was cooled in an ice bath. The solid precipitate was filtered, rinsed with a minimal amount of cold water, and dried in a vacuum oven at 50° C. overnight, providing the title compound (697 mg, 90%) as a white solid: ¹H NMR (500 MHz, CDCl₃) δ 12.39 (br s, 1H), 7.39 (d, J=6.5 Hz, 1H), 7.23 (d, J=8.5 Hz, 1H), 7.13-7.11 (m, 2H), 6.51 (d, J=1.5 Hz, 1H), 6.24 (dd, J=6.8, 1.8 Hz, 1H), 2.33 (s, 3H); ESI MS m/z 270 [M+H]⁺.

### c) tert-Butyl 7-(4-(2-methyl-4-(trifluoromethoxy)phenyl)-2-oxopyridin-1(2H)-yl)-3,4-dihydrobenzofuro[3,2-c]pyridine-2(1H)-carboxylate

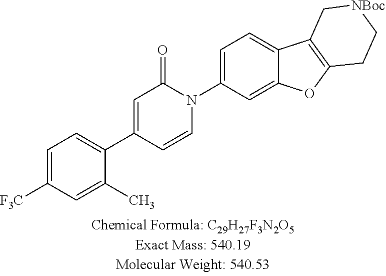 [SMILES: Cc1cc(C(F)(F)F)ccc1-c1ccn(-c2ccc3c4c(oc3c2)CCN(C(=O)OC(C)(C)C)C4)c(=O)c1]

tert-Butyl 7-bromo-3,4-dihydrobenzofuro[3,2-c]pyridine-2(1H)-carboxylate (177 mg, 0.503 mmol) and 4-(2-methyl-4-(trifluoromethoxy)phenyl)pyridin-2(1H)-one (135 mg, 0.501 mmol) were reacted according to Example 3 (step b) to provide the title compound (246 mg, 91%) as a light yellow solid: ¹H NMR (500 MHz, CDCl₃) δ 7.56 (d, J=2.0 Hz, 1H), 7.53 (d, J=8.0 Hz, 1H), 7.43 (d, J=7.0 Hz, 1H), 7.29 (d, J=8.0 Hz, 1H), 7.30-7.27 (m, 1H), 7.15-7.12 (m, 2H), 6.60 (d, J=1.5 Hz, 1H), 6.22 (dd, J=7.0, 2.0 Hz, 1H), 4.59 (s, 2H), 3.85 (s, 2H), 2.89 (s, 2H), 2.40 (s, 3H), 1.52 (s, 9H); ESI MS m/z 541 [M+H]⁺.

### d) 4-(2-Methyl-4-(trifluoromethoxy)phenyl)-1-(1,2,3,4-tetrahydrobenzofuro[3,2-c]pyridin-7-yl)pyridin-2(1H)-one

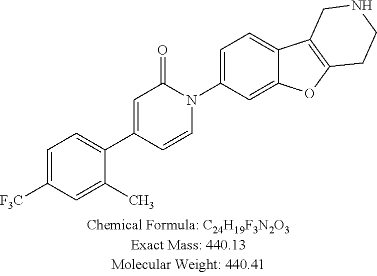 [SMILES: Cc1cc(C(F)(F)F)ccc1-c1ccn(-c2ccc3c4c(oc3c2)CCNC4)c(=O)c1]

A solution of tert-butyl 7-(4-(2-methyl-4-(trifluoromethoxy)phenyl)-2-oxopyridin-1(2H)-yl)-3,4-dihydrobenzofuro[3,2-c]pyridine-2(1H)-carboxylate (244 mg, 0.451 mmol) in MeOH (4.5 mL) was treated with 2 N HCl in Et₂O (9 mL), and the resulting solution was stirred at ambient temperature for 16 h and then concentrated in vacuo. The resultant HCl salt was converted to the corresponding free base using an SCX-2 cartridge to provide the title compound (194 mg, 98%) as a light yellow semi-solid: ¹H NMR (500 MHz, CDCl₃) δ 7.54 (d, J=1.5 Hz, 1H), 7.48 (d, J=8.5 Hz, 1H), 7.43 (d, J=7.0 Hz, 1H), 7.30-7.27 (m, 2H), 7.14-7.12 (m, 2H), 6.60 (d, J=2.0 Hz, 1H), 6.22 (dd, J=7.0, 2.0 Hz, 1H), 4.01 (s, 2H), 3.27 (t, J=5.8 Hz, 2H), 2.83 (t, J=5.8 Hz, 2H), 2.40 (s, 3H); ESI MS m/z 441 [M+H]⁺.

### e) 4-(2-Methyl-4-(trifluoromethoxy)phenyl)-1-(1,2,3,4-tetrahydrobenzofuro[3,2-c]pyridin-7-yl)pyridin-2(1H)-one hydrochloride

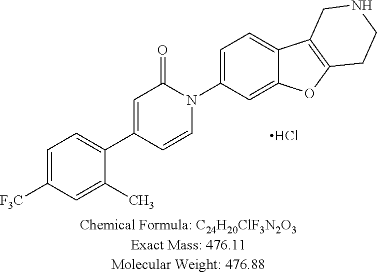 [SMILES: Cc1cc(C(F)(F)F)ccc1-c1ccn(-c2ccc3c4c(oc3c2)CCNC4)c(=O)c1 ; Cl]

A solution of 4-(2-methyl-4-(trifluoromethoxy)phenyl)-1-(1,2,3,4-tetrahydrobenzofuro[3,2-c]pyridin-7-yl)pyridin-2(1H)-one (185 mg, 0.420 mmol) in MeOH (5 mL) was treated with 2 N HCl in Et₂O (0.21 mL, 0.42 mmol), and the resulting suspension was concentrated. The solid was suspended in H₂O (3 mL), frozen, and lyophilized overnight to provide the title compound (198 mg, 99%) as a white solid: mp 299-301° C.; ¹H NMR (500 MHz, DMSO-d₆) δ 9.64 (br s, 2H), 7.81 (d, J=1.5 Hz, 1H), 7.76-7.73 (m, 2H), 7.43 (d, J=8.5 Hz, 1H), 7.40-7.38 (m, 2H), 7.30 (d, J=8.0 Hz, 1H), 6.46 (d, J=1.5 Hz, 1H), 6.39 (dd, J=7.0, 2.0 Hz, 1H), 4.37 (s, 2H), 3.56 (t, J=5.8 Hz, 2H), 3.14 (s, 2H), 2.39 (s, 3H); ESI MS m/z 441 [M+H]⁺; HPLC (Method A)>99% (AUC), t_{R}=15.4 min.

### Example 115

### Preparation of 1-(1,2,3,4-Tetrahydrobenzofuro[3,2-c]pyridin-7-yl)-4-((5-(trifluoromethyl)pyridin-2-yl)methoxy)pyridin-2(1H)-one hydrochloride

### a) 4-((5-(Trifluoromethyl)pyridin-2-yl)methoxy)pyridin-2(1H)-one

CAS Registry Number 1184949-45-8

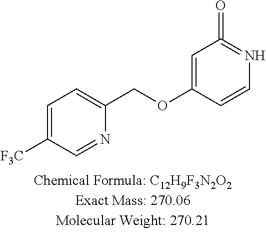

This compound was prepared in accordance with the procedure described in PCT Publication No. WO 2009/103478 Stenkamp, Dirk et al., which is hereby incorporated by reference in its entirety.

### b) tert-Butyl 7-(2-oxo-4-((5-(trifluoromethyl)pyridin-2-yl)methoxy)pyridin-1(2H)-yl)-3,4-dihydrobenzofuro[3,2-c]pyridine-2(1H)-carboxylate

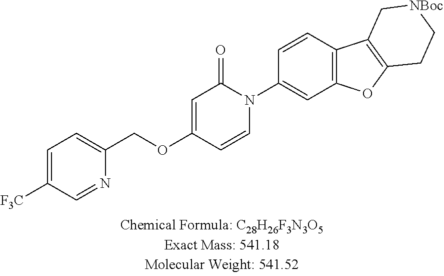 [SMILES: CC(C)(C)OC(=O)N1CCc2oc3cc(-n4ccc(OCc5ccc(C(F)(F)F)cn5)cc4=O)ccc3c2C1]

tert-Butyl 7-bromo-3,4-dihydrobenzofuro[3,2-c]pyridine-2(1H)-carboxylate (167 mg, 0.474 mmol) and 4-((5-(Trifluoromethyl)pyridin-2-yl)methoxy)pyridin-2(1H)-one (128 mg, 0.474 mmol) were reacted according to Example 12 (step c) to provide the title compound (70 mg, 26%) as a white foam: ESI MS m/z 542 [M+H]⁺.

### c) 1-(1,2,3,4-Tetrahydrobenzofuro[3,2-c]pyridin-7-yl)-4-((5-(trifluoromethyl)pyridin-2-yl)methoxy)pyridin-2(1H)-one hydrochloride

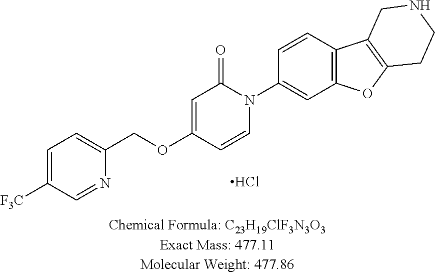 [SMILES: O=c1cc(OCc2ccc(C(F)(F)F)cn2)ccn1-c1ccc2c3c(oc2c1)CCNC3 ; Cl]

tert-Butyl 7-(2-oxo-4-((5-(trifluoromethyl)pyridin-2-yl)methoxy)pyridin-1(2H)-yl)-3,4-dihydrobenzofuro[3,2-c]pyridine-2(1H)-carboxylate (70 mg, 0.13 mmol) was deprotected and converted to the hydrochloride according to Example 12 (step d) to provide the title compound (61 mg, 100%) as a white solid: ¹H NMR (500 MHz, CD₃OD) δ 8.96 (d, J=2.1, Hz, 1H), 8.27 (dd, J=8.3, 2.4 Hz, 1H), 7.86 (d, J=8.2 Hz, 1H), 7.73 (d, J=7.6 Hz, 1H), 7.70 (d, J=8.2 Hz, 1H), 7.65 (d, J=1.8 Hz, 1H), 7.32 (dd, J=8.3, 1.8 Hz, 1H), 6.52 (dd, J=7.6, 2.7 Hz, 1H), 6.25 (d, J=2.7 Hz, 1H), 5.45 (s, 2H), 4.48 (t, J=1.9 Hz, 2H), 3.71 (t, J=6.2 Hz, 2H), 3.22 (t, J=6.2 Hz, 2H); ESI MS m/z 442 [M+H]⁺; HPLC (Method A) 96.3% (AUC), t_{R}=13.6 min.

### Example 116

### Preparation of 4-(Benzyloxy)-1-(2,3,4,5-tetrahydro-1H-benzo[4,5]thieno[2,3-c]azepin-7-yl)pyridin-2(1H)-one hydrochloride

### a) tert-Butyl 7-(4-(benzyloxy)-2-oxopyridin-1(2H)-yl)-4,5-dihydro-1H-benzo[4,5]thieno[2,3-c]azepine-2(3H)-carboxylate

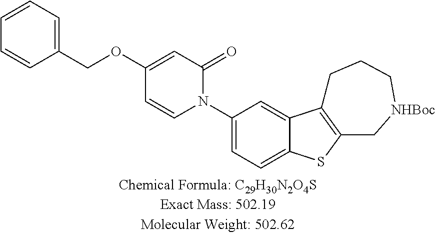

tert-Butyl 7-bromo-4,5-dihydro-1H-benzo[4,5]thieno[2,3-c]azepine-2(3H)-carboxylate (0.10 g, 0.26 mmol) and 4-benzyloxy pyridinone (53 mg, 0.26 mmol) were reacted according to Example 12 (step c) to provide the title compound (85 mg, 64%) as an off-white solid: ¹H NMR (400 MHz, CDCl₃) δ 7.85 (d, J=8.4 Hz, 1H), 7.59 (d, J=2.0 Hz, 1H), 7.48-7.34 (m, 5H), 7.32-7.22 (m, 2H), 6.12-6.03 (m, 2H), 5.06 (s, 2H), 4.70-4.54 (m, 2H), 3.83-3.68 (m, 2H), 2.97 (t, J=5.6 Hz, 2H), 1.98-1.89 (m, 2H), 1.46-1.36 (m, 9H); APCI MS m/z 503 [M+H]⁺.

### b) 4-(Benzyloxy)-1-(2,3,4,5-tetrahydro-1H-benzo[4,5]thieno[2,3-c]azepin-7-yl)pyridin-2(1H)-one hydrochloride

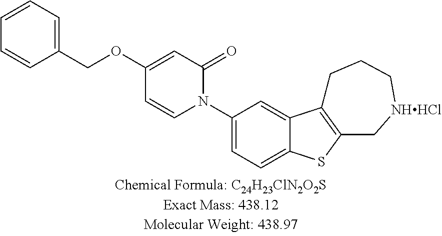

tert-Butyl 7-(4-(benzyloxy)-2-oxopyridin-1(2H)-yl)-4,5-dihydro-1H-benzo[4,5]thieno[2,3-c]azepine-2(3H)-carboxylate (85 mg, 0.16 mmol) and cone. HCl (0.7 mL) were reacted according to Example 74 (step b) to provide the title compound (60 mg, 83%) as an off white solid: ¹H NMR (300 MHz, DMSO-d₆) δ 9.54-9.40 (m, 2H), 8.07 (d, J=8.4 Hz, 1H), 7.82 (d, J=1.5 Hz, 1H), 7.65 (d, J=7.5 Hz, 1H), 7.53-7.32 (m, 6H), 6.15 (dd, J=7.8, 2.7 Hz, 1H), 6.00 (d, J=2.4 Hz, 1H), 5.16 (s, 2H), 4.62-4.52 (m, 2H), 3.54-3.41 (m, 2H), 3.17-3.05 (m, 2H), 2.06-1.91 (m, 2H); APCI MS m/z 403 [M+H]⁺; HPLC (Method C)>99% (AUC), t_{R}=20.93 min.

### Example 117

### Preparation of 4-(Benzyloxy)-1-(2,3,4,5-tetrahydro-1H-benzo[4,5]thieno[2,3-d]azepin-9-yl)pyridin-2(1H)-one hydrochloride

### a) tert-Butyl 9-(4-(benzyloxy)-2-oxopyridin-1(2H)-yl)-4,5-dihydro-1H-benzo[4,5]thieno[2,3-d]azepine-3(2H)-carboxylate

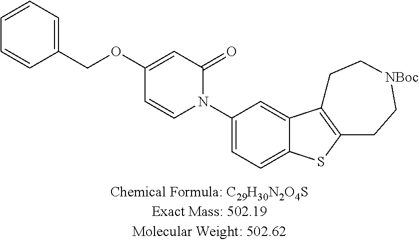 [SMILES: CC(C)(C)OC(=O)N1CCc2sc3ccc(-n4ccc(OCc5ccccc5)cc4=O)cc3c2CC1]

tert-Butyl 9-bromo-4,5-dihydro-1H-benzo[4,5]thieno[2,3-d]azepine-3(2H)-carboxylate (0.30 g, 0.78 mmol) and 4-benzyloxy pyridinone (158 mg, 0.785 mmol) were reacted according to Example 12 (step c) to provide the title compound (240 mg, 60%) as an off-white solid: ¹H NMR (400 MHz, CDCl₃) δ 7.85-7.78 (m, 1H), 7.58-7.52 (m, 1H), 7.46-7.34 (m, 5H), 7.31-7.22 (m, 2H), 6.12-6.03 (m, 2H), 5.06 (s, 2H), 3.77-3.61 (m, 4H), 3.17-2.94 (m, 4H), 1.49 (s, 9H); APCI MS m/z 503 [M+H]⁺.

### b) 4-(Benzyloxy)-1-(2,3,4,5-tetrahydro-1H-benzo[4,5]thieno[2,3-d]azepin-9-yl)pyridin-2(1H)-one hydrochloride

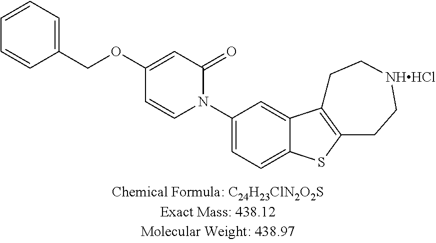

tert-Butyl 9-(4-(benzyloxy)-2-oxopyridin-1(2H)-yl)-4,5-dihydro-1H-benzo[4,5]thieno[2,3-d]azepine-3(2H)-carboxylate (240 mg, 0.47 mmol) and conc. HCl (0.5 mL) were reacted according to Example 74 (step b) to provide the title compound (196 mg, 97%) as an off white solid: ¹H NMR (400 MHz, DMSO-d₆) δ 9.76-9.64 (m, 2H), 8.01 (d, J=8.4 Hz, 1H), 7.74 (d, J=1.2 Hz, 1H), 7.62 (d, J=7.6 Hz, 1H), 7.52-7.34 (m, 5H), 7.31 (dd, J=8.4, 1.2 Hz, 1H), 6.14 (dd, J=7.6, 2.4 Hz, 1H), 6.01 (d, J=2.4 Hz, 1H), 5.16 (s, 2H), 3.42-3.21 (m, 8H); APCI MS m/z 403 [M+H]⁺; HPLC (Method C)>99% (AUC), t_{R}=21.01 min.

### Example 118

### Preparation of 4-Benzyloxy-1-(2,3,4,5-tetrahydro-1H-benzo[4,5]thieno[3,2-c]azepin-9-yl)pyridin-2(1H)-one hydrochloride

### a) tert-Butyl 4-(4-bromophenyl)thio-3-hydroxyazepane-1-carboxylate

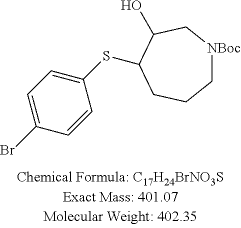 [SMILES: CC(C)(C)OC(=O)N1CCCC(Sc2ccc(Br)cc2)C(O)C1]

### tert-Butyl 8-oxa-3-azabicyclo[5.1.0]octane-3-carboxylate (4.60 g, 23.2 mmol) was reacted with 4-bromobenzenethiol (5.3 g, 28 mmol) according to Example 124 (step b) to provide the title compound (5.8 g, 62%) as a colorless oil: ¹H NMR (400 MHz, CDCl₃) δ 7.42 (d, J=7.6 Hz, 2H), 7.27 (d, J=10.8 Hz, 2H), 3.98 (br s, 0.6H), 3.85-3.55 (m, 2.7H), 3.45-2.87 (m, 3.7H), 2.14-2.08 (m, 1H), 1.87 (br s, 1H), 1.73-1.68 (m, 1H), 1.46 (s, 9H).

### b) tert-Butyl 4-(4-bromophenyl)thio-3-oxoazepane-1-carboxylate

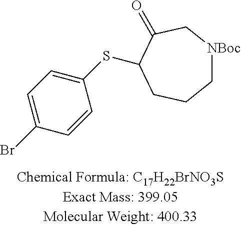 [SMILES: CC(C)(C)OC(=O)N1CCCC(Sc2ccc(Br)cc2)C(=O)C1]

tert-Butyl 4-(4-bromophenyl)thio-3-hydroxyazepane-1-carboxylate (5.80 g, 14.4 mmol) was reacted with Dess-Martin periodinane (12.2 g, 28.8 mmol) according to Example 124 (step c) to provide the title compound (4.7 g, 81%) as a colorless oil: ¹H NMR (300 MHz, CDCl₃) δ 7.41 (d, J=8.4 Hz, 2H), 7.30-7.22 (m, 2H), 4.48-4.33 (m, 1H), 4.01-3.92 (m, 2H), 3.58-3.53 (m, 1H), 3.22-3.11 (m, 1H), 2.05-2.02 (m, 3H), 1.82-1.74 (m, 1H), 1.53 (s, 5H), 1.48 (s, 4H).

### c) tert-Butyl 9-bromo-4,5-dihydro-1H-benzo[4,5]thieno[3,2-c]azepine-2(3H)-carboxylate

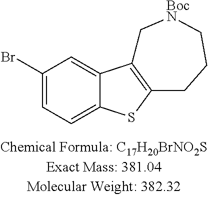 [SMILES: CC(C)(C)OC(=O)N1CCCc2sc3ccc(Br)cc3c2C1]

tert-Butyl 4-(4-bromophenyl)thio-3-oxoazepane-1-carboxylate (500 mg, 1.25 mmol) was reacted with polyphosphoric acid (2.5 g) according to Example 16 (step b) to provide the title compound (270 mg, 58%) as a colorless oil: ¹H NMR (300 MHz, CDCl₃) δ 7.80 (br s, 1H), 7.58-7.52 (m, 1H), 7.35-7.32 (m, 1H), 4.64 (s, 0.6H), 4.53 (s, 1.4H), 3.78-3.68 (m, 2H), 2.99 (br, 2H), 1.94 (br, 2H), 1.43 (s, 3H), 1.33 (s, 6H).

### d) tert-Butyl 9-(4-benzyloxy-2-oxopyridin-1(2H)-yl)-4,5-dihydro-1H-benzo[4,5]thieno[3,2-c]azepine-2(3H)-carboxylate

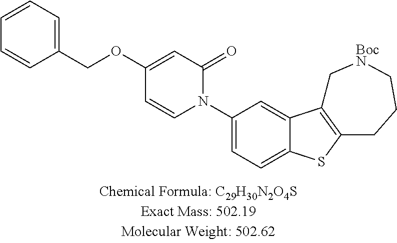 [SMILES: CC(C)(C)OC(=O)N1CCCc2sc3ccc(-n4ccc(OCc5ccccc5)cc4=O)cc3c2C1]

tert-Butyl 9-bromo-4,5-dihydro-1H-benzo[4,5]thieno[3,2-c]azepine-2(3H)-carboxylate (135 mg, 0.35 mmol) and 4-(benzyloxy)pyridin-2(1H)-one (86 mg. 0.43 mmol) were coupled according to Example 65 (step d) to provide the title compound (98 mg, 52%) as a colorless oil: ¹H NMR (300 MHz, CDCl₃) δ 7.83-7.77 (m, 1H), 7.64-7.58 (m, 1H), 7.44-7.30 (m, 4H), 7.26-7.23 (m, 3H), 6.07-6.03 (m, 2H), 5.06 (s, 2H), 4.68 (s, 0.6H), 4.59 (s, 1.4H), 3.76-3.70 (m, 2H), 3.01 (br s, 2H), 2.04-1.98 (m, 2H), 1.43 (s, 4H), 1.31 (s, 5H).

### d) 4-Benzyloxy-1-(2,3,4,5-tetrahydro-1H-benzo[4,5]thieno[3,2-c]azepin-9-yl)pyridin-2(1H)-one hydrochloride

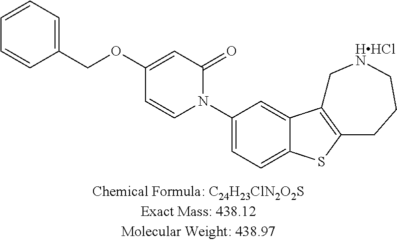

tert-Butyl 9-(4-benzyloxy-2-oxopyridin-1(2H)-yl)-4,5-dihydro-1H-benzo[4,5]thieno[3,2-c]azepine-2(3H)-carboxylate (84 mg, 0.16 mmol) was treated with concentrated aqueous HCl according to Example 65 (step e) to provide the title compound (62 mg, 88%) as a white solid: mp 176-178° C.; ¹H NMR (400 MHz, DMSO-d₆) δ 9.15 (br s, 2H), 8.04 (d, J=8.4 Hz, 1H), 7.94 (d, J=2.0 Hz, 1H), 7.59 (d, J=7.6 Hz, 1H), 7.49-7.32 (m, 6H), 6.15 (dd, J=7.6, 2.8 Hz, 1H), 6.00 (d, J=2.8 Hz, 1H), 5.17 (s, 2H), 4.54 (s, 2H), 3.48 (s, 2H), 3.19-3.16 (m, 2H), 2.01 (br s, 2H); ESI MS m/z 403 [M+H]⁺; HPLC (Method C)>99% (AUC), t_{R}=21.1 min.

### Example 119

### Preparation of 4-((5-Fluoropyridin-2-yl)methoxy)-1-(2,3,4,5-tetrahydro-1H-benzo[4,5]thieno[3,2-c]azepin-9-yl)pyridin-2(1H)-one hydrochloride

### a) tert-Butyl 9-(4-((5-fluoropyridin-2-yl)methoxy)-2-oxopyridin-1(2H)-yl)-4,5-dihydro-1H-benzo[4,5]thieno[3,2-c]azepine-2(3H)-carboxylate

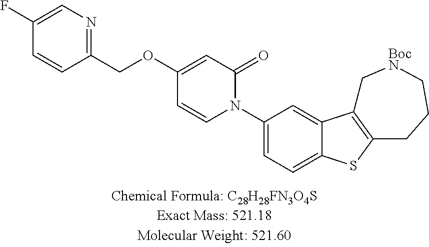 [SMILES: CC(C)(C)OC(=O)N1CCCc2sc3ccc(-n4ccc(OCc5ccc(F)cn5)cc4=O)cc3c2C1]

tert-Butyl 9-bromo-4,5-dihydro-1H-benzo[4,5]thieno[3,2-c]azepine-2(3H)-carboxylate (135 mg, 0.353 mmol) was reacted with 4-(5-fluoropyridin-2-ylmethyl)aminopyridin-2(1H)-one (95 mg, 0.42 mmol) according to Example 65 (step d) to provide the title compound (124 mg, 68%) as a colorless oil: ¹H NMR (300 MHz, CDCl₃) δ 8.49 (d, J=2.1 Hz, 1H), 7.83-7.78 (m, 1H), 7.62 (d, J=17.1 Hz, 1H), 7.50-7.46 (m, 2H), 7.33-7.23 (m, 2H), 6.11-6.06 (m, 2H), 5.17 (s, 2H), 4.68 (s, 0.8H), 4.60 (s, 1.2H), 3.76-3.71 (m, 2H), 3.05-3.01 (m, 2H), 2.04-2.00 (m, 2H), 1.44 (s, 5H), 1.31 (s, 4H).

### b) 4-((5-Fluoropyridin-2-yl)methoxy)-1-(2,3,4,5-tetrahydro-1H-benzo[4,5]thieno[3,2-c]azepin-9-yl)pyridin-2(1H)-one hydrochloride

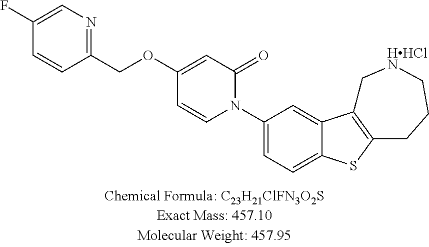

tert-Butyl 9-(4-((5-fluoropyridin-2-yl)methoxy)-2-oxopyridin-1(2H)-yl)-4,5-dihydro-1H-benzo[4,5]thieno[3,2-c]azepine-2(3H)-carboxylate (124 mg, 0.24 mmol) was treated with concentrated aqueous HCl according to Example 65 (step e) to provide the title compound (78 mg, 71%) as a white solid: mp 170-172° C.; ¹H NMR (400 MHz, DMSO-d₆) δ 9.29 (br s, 2H), 8.62 (d, J=3.2 Hz, 1H), 8.03 (d, J=8.4 Hz, 1H), 7.94 (d, J=2.0 Hz, 1H), 7.86-7.81 (m, 1H), 7.68-7.61 (m, 2H), 7.33 (dd, J=8.4, 2.0 Hz, 1H), 6.18 (dd, J=7.6, 2.8 Hz, 1H), 6.00 (d, J=2.8 Hz, 1H), 5.23 (s, 2H), 4.52 (br s, 2H), 3.47 (br s, 2H), 3.19-3.16 (m, 2H), 2.02 (br s, 2H); ESI MS m/z 422 [M+H]⁺; HPLC (Method C)>99% (AUC), t_{R}=19.8 min.

### Example 120

### Preparation of 4-(5-Fluoropyridin-2-ylmethoxy)-1-(2,3,4,5-tetrahydro-1H-benzo[4,5]thieno[2,3-c]azepin-7-yl)pyridin-2(1H)-one hydrochloride

### a) tert-Butyl 7-bromo-4,5-dihydro-1H-benzo[4,5]thieno[2,3-c]azepine-2(3H)-carboxylate

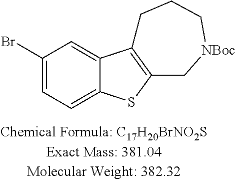 [SMILES: CC(C)(C)OC(=O)N1CCCc2c(sc3ccc(Br)cc23)C1]

A mixture of 7-bromo-2,3,4,5-tetrahydro-1H-benzo[4,5]thieno[2,3-c]azepine (169 mg, 0.599 mmol), triethylamine (0.42 mL, 3.0 mmol) and di-tert-butyl dicarbonate (340 mg, 1.50 mmol) in MeOH (10 mL) was stirred at ambient for 2 h. The mixture was concentrated in vacuo, and the residue was purified by flash column chromatography (silica gel, hexanes/EtOAc, 99:1 to 95:5) to afford the title compound (191 mg, 83%) as a colorless oil: ¹H NMR (300 MHz, CDCl₃) δ 7.76 (s, 1H), 7.61 (d, J=8.4 Hz, 1H), 7.37 (d, J=8.4 Hz, 1H), 4.64 (s, 0.6H), 4.55 (s, 1.4H), 3.77 (br s, 2H), 2.96-2.92 (m, 2H), 1.98-1.91 (m, 2H), 1.39 (s, 9H).

### b) tert-Butyl 7-(4-(5-fluoropyridin-2-ylmethoxy)-2-oxopyridin-1(2H)-yl)-4,5-dihydro-1H-benzo[4,5]thieno[2,3-c]azepine-2(3H)-carboxylate

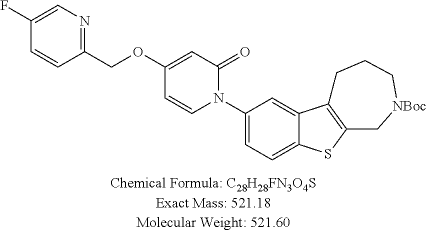 [SMILES: CC(C)(C)OC(=O)N1CCCc2c(sc3ccc(-n4ccc(OCc5ccc(F)cn5)cc4=O)cc23)C1]

tert-Butyl 7-bromo-4,5-dihydro-1H-benzo[4,5]thieno[2,3-c]azepine-2(3H)-carboxylate (95 mg, 0.25 mmol) and 4-(5-fluoropyridin-2-yl)methoxypyridin-2(1H)-one (60 mg, 0.27 mmol) were coupled according to Example 65 (step d) to provide the title compound (65 mg, 51%) as a colorless oil: ¹H NMR (300 MHz, CDCl₃) δ 8.49 (d, J=2.1 Hz, 1H), 7.84 (d, J=8.4 Hz, 1H), 7.59 (d, J=2.1 Hz, 1H), 7.51-7.46 (m, 2H), 7.32-7.26 (m, 2H), 6.12-6.06 (m, 2H), 5.17 (s, 2H), 4.66 (s, 0.6H), 4.58 (s, 1.4H), 3.77 (br s, 2H), 2.99-2.95 (m, 2H), 1.95-1.92 (m, 2H), 1.42 (s, 9H).

### c) 4-(5-Fluoropyridin-2-yl)methoxy-1-(2,3,4,5-tetrahydro-1H-benzo[4,5]thieno[2,3-c]azepin-7-yl)pyridin-2(1H)-one hydrochloride

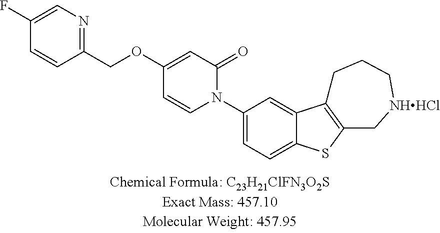

tert-Butyl 7-(4-(5-fluoropyridin-2-ylmethoxy)-2-oxopyridin-1(2H)-yl)-4,5-dihydro-1H-benzo[4,5]thieno[2,3-c]azepine-2(3H)-carboxylate (65 mg, 0.13 mmol) was treated with concentrated aqueous HCl according to Example 65 (step e) to provide the title compound (46 mg, 81%) as a white solid: mp 180-182° C.; ¹H NMR (300 MHz, DMSO-d₆) δ 9.35 (br s, 2H), 8.62 (d, J=3.0 Hz, 1H), 8.06 (d, J=8.7 Hz, 1H), 7.86-7.79 (m, 2H), 7.68-7.64 (m, 2H), 7.36 (dd, J=8.4, 1.8 Hz, 1H), 6.16 (dd, J=7.8, 2.7 Hz, 1H), 6.00 (d, J=2.7 Hz, 1H), 5.23 (s, 2H), 4.58 (s, 2H), 3.48 (d, J=1.8 Hz, 2H), 3.12-3.09 (m, 2H), 1.98 (br s, 2H); APCI MS m/z 422 [M+H]⁺; HPLC (Method C)>99% (AUC), t_{R}=19.6 min.

### Example 121

### Preparation of 4-(2-(5-Chloropyridin-2-yl)ethyl)-1-(1,2,3,4-tetrahydrobenzothieno[2,3-c]pyridin-7-yl)piperazin-2-one hydrochloride

### a) tert-Butyl 7-(4-(2-(5-chloropyridin-2-yl)ethyl)-2-oxopiperazin-1-yl)-3,4-dihydrobenzothieno[2,3-c]pyridine-2(1H)-carboxylate

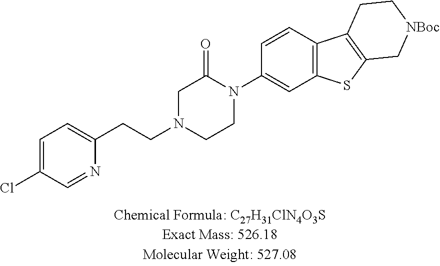 [SMILES: CC(C)(C)OC(=O)N1CCc2c(sc3cc(N4CCN(CCc5ccc(Cl)cn5)CC4=O)ccc23)C1]

tert-Butyl 7-bromo-3,4-dihydrobenzothieno[2,3-c]pyridine-2(1H)-carboxylate (130 mg 353 mg, mmol) and 4-(2-(5-chloropyridin-2-yl)ethyl)piperazin-2-one (84 mg, 0.35 mmol) were reacted according to Example 12 (step c) to provide the title compound (89 mg, 48%) as a white foam: ESI MS m/z [M+H]⁺.

### b) 4-(2-(5-Chloropyridin-2-yl)ethyl)-1-(1,2,3,4-tetrahydrobenzothieno[2,3-c]pyridin-7-yl)piperazin-2-one hydrochloride

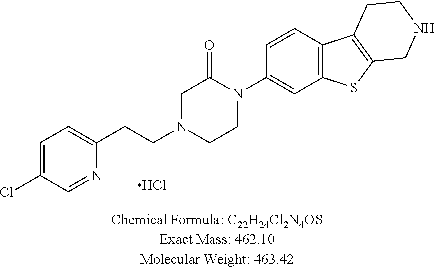 [SMILES: O=C1CN(CCc2ccc(Cl)cn2)CCN1c1ccc2c3c(sc2c1)CNCC3 ; Cl]

### tert-Butyl 7-(4-(2-(5-chloropyridin-2-yl)ethyl)-2-oxopiperazin-1-yl)-3,4-dihydrobenzothieno[2,3-c]pyridine-2(1H)-carboxylate (89 mg, 0.17 mmol) was deprotected and converted to the hydrochloride according to Example 12 (step d) to provide the title compound (53 mg, 68%) as a white solid: ¹H NMR (500 MHz, CD₃OD) δ 8.53 (d, J=2.2 Hz, 1H), 7.91 (d, J=1.5 Hz, 1H), 7.84-7.82 (dd, J=8.3, 2.5 Hz, 1H), 7.80 (d, J=8.5 Hz, 1H), 7.43-7.42 (m, 2H), 4.56 (s, 2H), 3.99-3.90 (m, 2H), 3.88-3.74 (m, 2H), 3.66 (t, J=6.2 Hz, 2H), 3.54-3.36 (m, 4H), 3.27-3.21 (m, 2H), 3.18 (t, J=6.2 Hz, 2H); ESI MS m/z 427 [M+H]⁺; HPLC (Method B) 98.2% (AUC), t_{R}=7.0 min.

### Example 122

### Preparation of 4-(Benzyloxy)-1-(2,3,4,5-tetrahydro-1H-benzothieno[3,2-c]azepin-8-yl)pyridin-2(1H)-one hydrochloride

### a) tert-Butyl 8-(4-(benzyloxy)-2-oxopyridin-1(2H)-yl)-4,5-dihydro-1H-benzothieno[3,2-c]azepine-2(3H)-carboxylate

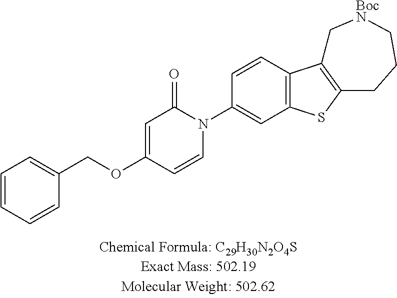 [SMILES: CC(C)(C)OC(=O)N1CCCc2sc3cc(-n4ccc(OCc5ccccc5)cc4=O)ccc3c2C1]

tert-Butyl 8-bromo-4,5-dihydro-1H-benzothieno[3,2-c]azepine-2(3H)-carboxylate (230 mg, 0.602 mmol) and 4-benzyloxy pyridinone (61 mg, 0.30 mmol) were reacted according to Example 12 (step c) to provide the title compound (75 mg, 50%) as a white foam: ESI MS m/z 503 [M+H]⁺.

### b) 4-(Benzyloxy)-1-(2,3,4,5-tetrahydro-1H-benzothieno[3,2-c]azepin-8-yl)pyridin-2(1H)-one hydrochloride

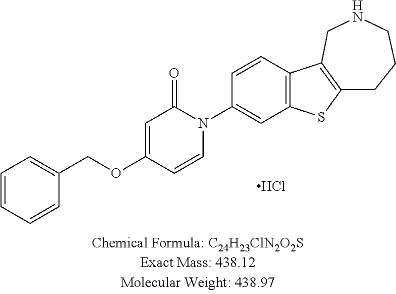 [SMILES: O=c1cc(OCc2ccccc2)ccn1-c1ccc2c3c(sc2c1)CCCNC3 ; Cl]

tert-Butyl 8-(4-(benzyloxy)-2-oxopyridin-1(2H)-yl)-4,5-dihydro-1H-benzothieno[3,2-c]azepine-2(3H)-carboxylate (75 mg, 0.15 mmol) was deprotected and converted to the hydrochloride according to Example 12 (step d) to provide the title compound (30 mg, 66%) as a white solid: ¹H NMR (500 MHz, DMSO-d₆) δ 9.19 (s, 2H), 8.00 (d, J=1.8 Hz, 1H), 7.95 (d, J=8.6 Hz, 1H), 7.61 (d, J=7.6 Hz, 1H), 7.47-7.36 (m, 6H), 6.14-6.12 (dd, J=7.7, 2.7 Hz, 1H), 5.99 (d, J=2.7 Hz, 1H), 5.15 (s, 2H), 4.58-4.52 (m, 2H), 3.52-3.45 (m, 2H), 3.20-3.13 (m, 2H), 2.06-1.97 (m, 2H); ESI MS m/z 403 [M+H]⁺; HPLC (Method B)>99% (AUC), t_{R}=15.9 min.

### Example 123

### Preparation of 4-((5-Chloropyridin-2-yl)methoxy)-1-(2,3,4,5-tetrahydro-1H-benzothieno[3,2-c]azepin-8-yl)pyridin-2(1H)-one hydrochloride

### a) tert-Butyl 8-(4-((5-chloropyridin-2-yl)methoxy)-2-oxopyridin-1(2H)-yl)-4,5-dihydro-1H-benzothieno[3,2-c]azepine-2(3H)-carboxylate

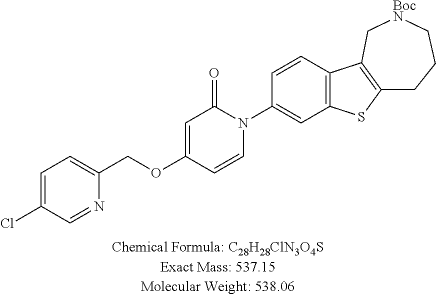 [SMILES: CC(C)(C)OC(=O)N1CCCc2sc3cc(-n4ccc(OCc5ccc(Cl)cn5)cc4=O)ccc3c2C1]

tert-Butyl 8-bromo-4,5-dihydro-1H-benzothieno[3,2-c]azepine-2(3H)-carboxylate (230 mg, 0.602 mmol) and 4-((5-chloropyridin-2-yl)methoxy)pyridin-2(1H)-one (71 mg, 0.30 mmol) were reacted according to Example 12 (step c) to provide the title compound (74 mg, 46%) as a white foam: ESI MS m/z 538 [M+H]⁺.

### b) 4-((5-Chloropyridin-2-yl)methoxy)-1-(2,3,4,5-tetrahydro-1H-benzothieno[3,2-c]azepin-8-yl)pyridin-2(1H)-one hydrochloride

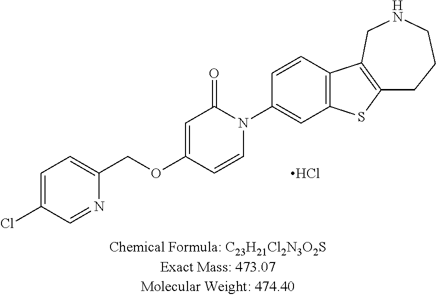 [SMILES: O=c1cc(OCc2ccc(Cl)cn2)ccn1-c1ccc2c3c(sc2c1)CCCNC3 ; Cl]

tert-Butyl 8-(4-((5-chloropyridin-2-yl)methoxy)-2-oxopyridin-1(2H)-yl)-4,5-dihydro-1H-benzothieno[3,2-c]azepine-2(3H)-carboxylate (74 mg, 0.14 mmol) was deprotected and converted to the hydrochloride according to Example 12 (step d) to provide the title compound (60 mg, 91%) as a white solid: ¹H NMR (500 MHz, DMSO-d₆) δ 9.20 (s, 2H), 8.33 (d, J=2.4 Hz, 1H), 8.04-8.02 (dd, J=8.3, 2.5 Hz, 1H), 8.00 (d, J=1.9 Hz, 1H), 7.95 (d, J=8.7 Hz, 1H), 7.67-7.60 (m, 2H), 7.41-7.37 (dd, J=8.6, 1.9 Hz, 1H), 6.18-6.16 (dd, J=7.6, 2.7 Hz, 1H), 5.97 (d, J=2.7 Hz, 1H), 5.23 (s, 2H), 4.57-4.52 (m, 2H), 3.52-3.46 (m, 2H), 3.20-3.13 (m, 2H), 2.06-1.97 (m, 2H); ESI MS m/z 438 [M+H]⁺; HPLC (Method B) 98.7% (AUC), t_{R}=16.0 min.

### Example 124

### Preparation of 4-((5-Fluoropyridin-2-yl)methoxy)-1-(2,3,4,5-tetrahydro-1H-benzothieno[3,2-c]azepin-8-yl)pyridin-2(1H)-one hydrochloride

### a) tert-Butyl 8-oxa-3-azabicyclo[5.1.0]octane-3-carboxylate

CAS Registry Number 281219-26-9

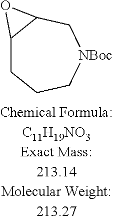 [SMILES: CC(C)(C)OC(=O)N1CCCC2OC2C1]

This compound was prepared in accordance with the procedure described in US Publication No. 2003/0144175 to Marquis et al., which is hereby incorporated by reference in its entirety.

### b) tert-Butyl 4-(3-bromophenylthio)-3-hydroxyazepane-1-carboxylate

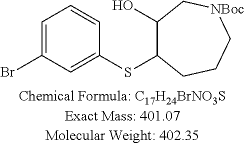 [SMILES: CC(C)(C)OC(=O)N1CCCC(Sc2cccc(Br)c2)C(O)C1]

3-Bromothiophenol (1.43 g, 7.57 mmol), NaOH (275 mg, 6.88 mmol) and tert-butyl 8-oxa-3-azabicyclo[5.1.0]octane-3-carboxylate (1.70 g, 7.99 mmol) were combined in MeOH (25 mL) and heated to reflux for 2.5 h. The mixture was concentrated and partitioned between brine (50 mL) and CH₂Cl₂ (50 mL). The organic layer was concentrated and purified by flash chromatography (24 g ISCO column, hexanes/EtOAc, 100:0 to 40:60) to provide the title compound (1.50 g, 54%) as a white solid: ESI MS m/z 424/426 [M+Na]⁺.

### c) tert-Butyl 4-(3-bromophenylthio)-3-oxoazepane-1-carboxylate

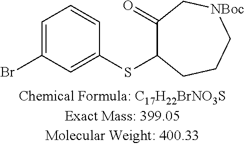 [SMILES: CC(C)(C)OC(=O)N1CCCC(Sc2cccc(Br)c2)C(=O)C1]

tert-Butyl 4-(3-bromophenylthio)-3-hydroxyazepane-1-carboxylate (1.50 g, 3.72 mmol) and Dess-Martin periodinane (1.89 g, 4.46 mmol) were combined in CH₂Cl₂ (20 mL) and stirred for 16 h. Water (30 mL), saturated NaHCO₃ solution (30 mL) and saturated Na₂S₂O₃ solution (30 mL) were added, and the mixture was stirred for 1 h. The organic layer was removed, dried over sodium sulfate and concentrated to provide the title compound (1.40 g, 94%) as a yellow oil: ESI MS m/z 400 [M+H]⁺.

### d) tert-Butyl 8-bromo-4,5-dihydro-1H-benzothieno[3,2-c]azepine-2(3H)-carboxylate

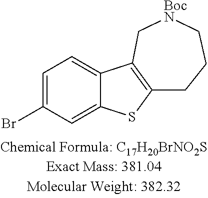 [SMILES: CC(C)(C)OC(=O)N1CCCc2sc3cc(Br)ccc3c2C1]

tert-Butyl 4-(3-bromophenylthio)-3-oxoazepane-1-carboxylate (1.40 g, 3.50 mmol) was reacted according to Example 16 (step b) to provide the title (700 mg, 52%) compound, containing undesired isomer, as a colorless oil ESI MS m/z 382 [M+H]⁺.

### e) tert-Butyl 8-(4-((5-fluoropyridin-2-yl)methoxy)-2-oxopyridin-1(2H)-yl)-4,5-dihydro-1H-benzothieno[3,2-c]azepine-2(3H)-carboxylate

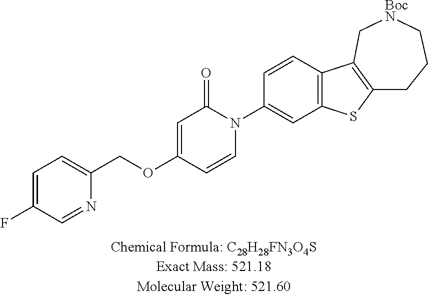 [SMILES: CC(C)(C)OC(=O)N1CCCc2sc3cc(-n4ccc(OCc5ccc(F)cn5)cc4=O)ccc3c2C1]

tert-Butyl 8-bromo-4,5-dihydro-1H-benzothieno[3,2-c]azepine-2(3H)-carboxylate (230 mg, 0.602 mmol) and 4-((5-fluoropyridin-2-yl)methoxy)pyridin-2(1H)-one (66 mg, 0.30 mmol) were reacted according to Example 12 (step c) to provide the title compound (90 mg, 57%) as a colorless oil: ESI MS m/z 522 [M+H]⁺.

### e) 4-((5-Fluoropyridin-2-yl)methoxy)-1-(2,3,4,5-tetrahydro-1H-benzothieno[3,2-c]azepin-8-yl)pyridin-2(1H)-one hydrochloride

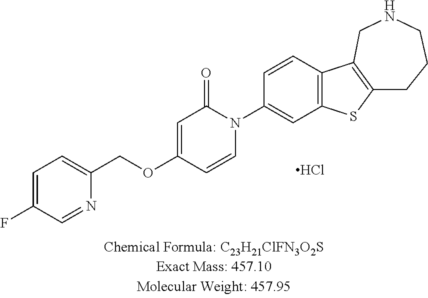 [SMILES: O=c1cc(OCc2ccc(F)cn2)ccn1-c1ccc2c3c(sc2c1)CCCNC3 ; Cl]

tert-Butyl 8-(4-((5-fluoropyridin-2-yl)methoxy)-2-oxopyridin-1(2H)-yl)-4,5-dihydro-1H-benzothieno[3,2-c]azepine-2(3H)-carboxylate (90 mg, 0.17 mmol) was deprotected and converted to the hydrochloride according to Example 12 (step d) to provide the title compound (48 mg, 67%) as a white solid: ¹H NMR (500 MHz, DMSO-d₆) δ 9.26 (s, 2H), 8.61 (d, J=2.9 Hz, 1H), 8.00 (d, J=1.9 Hz, 1H), 7.95 (d, J=8.6 Hz, 1H), 7.84-7.80 (td, J=8.7, 2.9 Hz, 1H), 7.67-7.62 (m, 2H), 7.41-7.37 (dd, J=8.6, 1.9 Hz, 1H), 6.17-6.15 (dd, J=7.6, 2.7 Hz, 1H), 5.99 (d, J=2.7 Hz, 1H), 5.22 (s, 2H), 4.57-4.51 (m, 2H), 3.52-3.46 (m, 2H), 3.18-3.16 (m, 2H), 2.05-1.98 (m, 2H); ESI MS m/z 422 [M+H]⁺; HPLC (Method B)>99% (AUC), t_{R}=11.4 min.

### Example 125

### Preparation of 4-((5-Chloropyridin-2-yl)ethyl)-1-(1,2,3,4-tetrahydrobenzothieno[2,3-c]pyridin-7-yl)pyridin-2(1H)-one hydrochloride

### a) tert-Butyl 7-(4-((5-chloropyridin-2-yl)ethyl)-2-oxopyridin-1(2H)-yl)-3,4-dihydrobenzothieno[2,3-c]pyridine-2(1H)-carboxylate

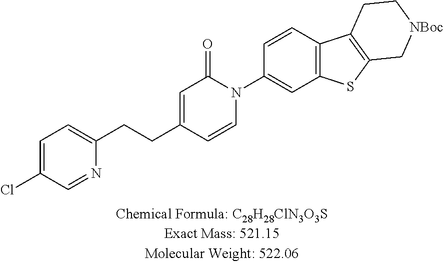 [SMILES: CC(C)(C)OC(=O)N1CCc2c(sc3cc(-n4ccc(CCc5ccc(Cl)cn5)cc4=O)ccc23)C1]

tert-Butyl 7-bromo-3,4-dihydrobenzothieno[2,3-c]pyridine-2(1H)-carboxylate (119 mg, 0.323 mmol) and 4-(2-(5-chloropyridin-2-yl)ethyl)pyridin-2(1H)-one (76.0 mg, 0.323 mmol) were reacted according to Example 12 (step c) to provide the title compound (131 mg, 78%) as a white foam: ESI MS m/z 522 [M+H]⁺.

### b) 4-((5-Chloropyridin-2-yl)ethyl)-1-(1,2,3,4-tetrahydrobenzothieno[2,3-c]pyridin-7-yl)pyridin-2(1H)-one hydrochloride

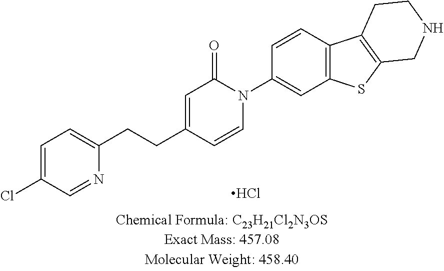 [SMILES: O=c1cc(CCc2ccc(Cl)cn2)ccn1-c1ccc2c3c(sc2c1)CNCC3 ; Cl]

tert-Butyl 7-(4-((5-chloropyridin-2-yl)ethyl)-2-oxopyridin-1(2H)-yl)-3,4-dihydrobenzothieno[2,3-c]pyridine-2(1H)-carboxylate (131 mg, 0.251 mmol) was deprotected and converted to the hydrochloride according to Example 12 (step d) to provide the title compound (82 mg, 55%) as a white solid: ¹H NMR (500 MHz, DMSO-d₆) δ 9.74 (s, 2H), 8.57 (d, J=2.5 Hz, 1H), 8.07 (d, J=1.8 Hz, 1H), 7.88-7.86 (dd, J=8.3, 2.6 Hz, 1H), 7.82 (d, J=8.5 Hz, 1H), 7.60 (d, J=7.0 Hz, 1H), 7.43-7.40 (m, 2H), 6.32-6.28 (m, 2H), 4.51-4.45 (m, 2H), 3.54-3.47 (m, 2H), 3.12-3.05 (m, 4H), 2.92-2.89 (m, 2H); ESI MS m/z 421 [M+H]⁺; HPLC (Method B)>99% (AUC), t_{R}=15.8 min.

### Example 126

### Preparation of 4-((5-Fluoropyridin-2-yl)ethyl)-1-(1,2,3,4-tetrahydrobenzothieno[2,3-c]pyridin-7-yl)pyridin-2(1H)-one hydrochloride

### a) tert-Butyl 7-(4-((5-fluoropyridin-2-yl)ethyl)-2-oxopyridin-1(2H)-yl)-3,4-dihydrobenzothieno[2,3-c]pyridine-2(1H)-carboxylate

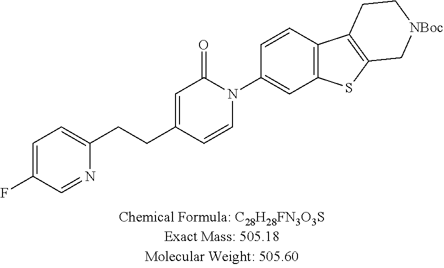 [SMILES: CC(C)(C)OC(=O)N1CCc2c(sc3cc(-n4ccc(CCc5ccc(F)cn5)cc4=O)ccc23)C1]

tert-Butyl 7-bromo-3,4-dihydrobenzothieno[2,3-c]pyridine-2(1H)-carboxylate (101 mg, 0.274 mmol) and 4-(2-(5-fluoropyridin-2-yl)ethyl)pyridin-2(1H)-one (60 mg, 0.27 mmol) were reacted according to Example 12 (step c) to provide the title compound (90 mg, 66%) as a white solid: ESI MS m/z 508 [M+H]⁺.

### b) 4-((5-Fluoropyridin-2-yl)ethyl)-1-(1,2,3,4-tetrahydrobenzothieno[2,3-c]pyridin-7-yl)pyridin-2(1H)-one hydrochloride

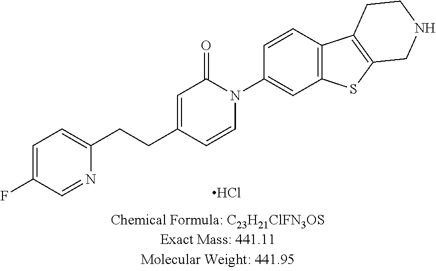 [SMILES: O=c1cc(CCc2ccc(F)cn2)ccn1-c1ccc2c3c(sc2c1)CNCC3 ; Cl]

tert-Butyl 7-(4-((5-fluoropyridin-2-yl)ethyl)-2-oxopyridin-1(2H)-yl)-3,4-dihydrobenzothieno[2,3-c]pyridine-2(1H)-carboxylate (90 mg, 0.18 mmol) was deprotected and converted to the hydrochloride according to Example 12 (step d) to provide the title compound (61 mg, 76%) as a white solid: ¹H NMR (500 MHz, DMSO-d₆) δ 9.70 (s, 2H), 8.52 (d, J=2.9 Hz, 1H), 8.07 (d, J=1.8 Hz, 1H), 7.82 (d, J=8.5 Hz, 1H), 7.71-7.65 (td, J=8.7, 3.0 Hz, 1H), 7.60 (d, J=7.0 Hz, 1H), 7.46-7.41 (m, 2H), 6.32-6.28 (m, 2H), 4.52-4.46 (m, 2H), 3.54-3.47 (m, 2H), 3.12-3.04 (m, 4H), 2.94-2.89 (m, 2H); ESI MS m/z 406 [M+H]⁺; HPLC (Method B)>99% (AUC), t_{R}=10.1 min.

### Example 127

### Preparation of 4-Phenethyl-1-(1,2,3,4-tetrahydrobenzothieno[2,3-c]pyridin-7-yl)pyridin-2(1H)-one hydrochloride

### a) 4-Phenethylpyridin-2(1H)-one

CAS Registry Number 16097-16-8

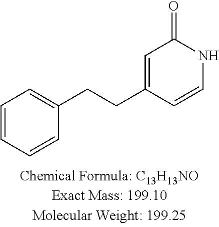 [SMILES: O=c1cc(CCc2ccccc2)cc[nH]1]

This compound was prepared in accordance with the procedure described in PCT Publication No. WO 2009/089482 to Guzzo et al., which is hereby incorporated by reference in its entirety.

### b) tert-Butyl 7-(2-oxo-4-phenethylpyridin-1(2H)-yl)-3,4-dihydrobenzothieno[2,3-c]pyridine-2(1H)-carboxylate

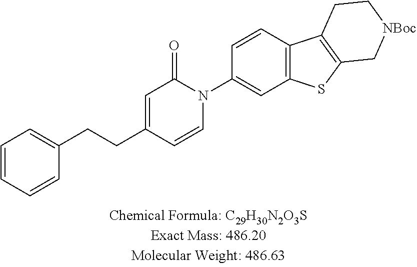 [SMILES: CC(C)(C)OC(=O)N1CCc2c(sc3cc(-n4ccc(CCc5ccccc5)cc4=O)ccc23)C1]

tert-Butyl 7-bromo-3,4-dihydrobenzothieno[2,3-c]pyridine-2(1H)-carboxylate (160 mg, 0.435 mmol) and 4-phenethylpyridin-2(1H)-one (86 mg, 0.44 mmol) were reacted according to Example 12 (step c) to provide the title compound (130 mg, 62%) as a white solid: ESI MS m/z 487 [M+H]⁺.

### c) 4-Phenethyl-1-(1,2,3,4-tetrahydrobenzothieno[2,3-c]pyridin-7-yl)pyridin-2(1H)-one hydrochloride

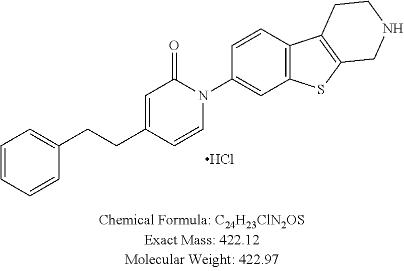 [SMILES: O=c1cc(CCc2ccccc2)ccn1-c1ccc2c3c(sc2c1)CNCC3 ; Cl]

tert-Butyl 7-(2-oxo-4-phenethylpyridin-1(2H)-yl)-3,4-dihydrobenzothieno[2,3-c]pyridine-2(1H)-carboxylate (130 mg, 0.267 mmol) was deprotected and converted to the hydrochloride according to Example 12 (step d) to provide the title compound (83 mg, 73%) as a white solid: ¹H NMR (500 MHz, DMSO-d₆) δ 9.78 (s, 2H), 8.07 (d, J=1.8 Hz, 1H), 7.82 (d, J=8.4 Hz, 1H), 7.62 (d, J=6.9 Hz, 1H), 7.46-7.41 (dd, J=8.5, 1.8 Hz, 1H), 7.33-7.26 (m, 4H), 7.23-7.18 (m, 1H), 6.33-6.31 (m, 2H), 4.52-4.45 (m, 2H), 3.53-3.45 (m, 2H), 3.09-3.07 (m, 2H), 2.94-2.88 (m, 2H), 2.80-2.72 (m, 2H); ESI MS m/z 387 [M+H]⁺; HPLC (Method B)>99% (AUC), t_{R}=16.0 min.

### Example 128

### Preparation of 4-(Benzyloxy)-1-(7,8,9,10-tetrahydro-6H-7,10-epiminocyclohepta[b]benzofuran-3-yl)pyridin-2(1H)-one hydrochloride

### a) O-(3-Bromophenyl)hydroxylamine hydrochloride

CAS Registry Number 937716-47-7

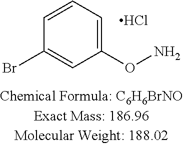

3-Bromophenol (27.0 g, 156 mmol) and hydroxylamine-O-sulfonic acid (4.4 g, 39 mmol) were reacted according to Example 2 (step a) to provide the title compound (3.8 g, 43%) as a pink-brown solid: ¹H NMR (300 MHz, DMSO-d₆) δ 9.95 (br s, 3H), 7.43 (t, J=1.8 Hz, 1H), 7.34-7.19 (m, 2H), 7.18-7.11 (m, 1H).

### b) tert-Butyl 3-bromo-7,8,9,10-tetrahydro-6H-7,10-epiminocyclohepta[b]benzofuran-11-carboxylate

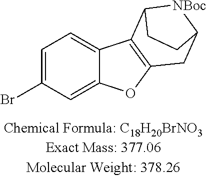 [SMILES: CC(C)(C)OC(=O)N1C2CCC1c1c(oc3cc(Br)ccc13)C2]

O-(3-Bromophenyl)hydroxylamine hydrochloride (2.0 g, 8.9 mmol) and tert-butyl 3-oxo-8-azabicyclo[3.2.1]octane-8-carboxylate (1.72 g, 10.69 mmol) were reacted according to Example 2 (step b) to provide the title compound (1.2 g, 36%) as an off-white solid: ¹H NMR (300 MHz, CDCl₃) δ 7.57 (br s, 1H), 7.37-7.28 (m, 2H), 5.27-4.98 (m, 1H), 4.78-4.53 (m, 1H), 3.58-3.22 (m, 1H), 2.53 (d, J=16.5 Hz, 1H), 2.42-2.11 (m, 2H), 2.03-1.92 (m, 1H), 1.74-1.59 (m, 1H), 1.40 (br s, 9H).

### c) tert-Butyl 3-(4-(benzyloxy)-2-oxopyridin-1(2H)-yl)-7,8,9,10-tetrahydro-6H-7,10-epiminocyclohepta[b]benzofuran-11-carboxylate

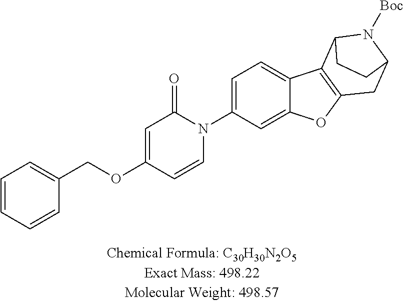 [SMILES: CC(C)(C)OC(=O)N1C2CCC1c1c(oc3cc(-n4ccc(OCc5ccccc5)cc4=O)ccc13)C2]

tert-Butyl 3-bromo-7,8,9,10-tetrahydro-6H-7,10-epiminocyclohepta[b]benzofuran-11-carboxylate (0.3 g, 0.79 mmol) and 4-benzyloxy pyridinone (0.16 g, 0.79 mmol) were reacted according to Example 74 (step a) to provide the title compound (40 mg, 10%) as an off-white solid: ¹H NMR (300 MHz, CDCl₃) δ 7.53 (d, J=8.1 Hz, 1H), 7.47-7.34 (m, 6H), 7.32-7.24 (m, 1H), 7.23-7.14 (m, 1H), 6.12-6.01 (m, 2H), 5.32-5.09 (m, 1H), 5.05 (s, 2H), 4.80-4.53 (m, 1H), 3.60-3.30 (m, 1H), 2.56 (d, J=17.4 Hz, 1H), 2.40-1.94 (m, 3H), 1.74-1.50 (m, 1H), 1.49-1.34 (m, 9H); APCI MS m/z 499 [M+H]⁺.

### d) 4-(Benzyloxy)-1-(7,8,9,10-tetrahydro-6H-7,10-epiminocyclohepta[b]benzofuran-3-yl)pyridin-2(1H)-one hydrochloride

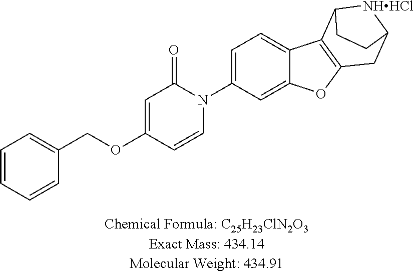

A solution of tert-butyl 3-(4-(benzyloxy)-2-oxopyridin-1(2H)-yl)-7,8,9,10-tetrahydro-6H-7,10-epiminocyclohepta[b]benzofuran-11-carboxylate (40 mg, 0.08 mmol) in MeOH (2.0 mL) was treated with 2 N HCl in Et₂O (0.5 mL), and the resulting solution was stirred at ambient temperature for 18 h. The solution was concentrated under reduced pressure, and the residue was diluted with water. The resulting solution was treated with 10% NaOH solution until the mixture was basic. The aqueous solution was extracted with CH₂Cl₂ (3×15 mL). The combined organic extracts were dried over Na₂SO₄, filtered and concentrated to dryness under reduced pressure. The crude product was purified by flash column chromatography (silica gel, CH₂Cl₂/MeOH, 100:0 to 90:10) to provide the free base which was dissolved in CH₂Cl₂ (2.0 mL) and treated with 2 N HCl in Et₂O at 0° C. The reaction was stirred at 0° C. for 20 min, and the resulting solid was filtered and washed with MTBE, then lyophilized from water to provide the title compound (82 mg, 94%) as an off white solid: ¹H NMR (300 MHz, CD₃OD) δ 7.74 (d, J=7.6 Hz, 1H), 7.65-7.52 (m, 2H), 7.50-7.24 (m, 6H), 6.35-6.25 (m, 1H), 6.11 (br s, 1H), 5.24 (s, 1H), 5.17 (s, 2H), 4.62-4.53 (m, 1H), 3.60-3.48 (m, 1H), 3.07 (d, J=17.6 Hz, 1H), 2.58-2.32 (m, 3H), 2.11-1.97 (m, 1H); APCI MS m/z 399 [M+H]⁺; HPLC (Method C)>99% (AUC), t_{R}=20.95 min.

### Example 129

### Preparation of 4-((5-Fluoropyridin-2-yl)methoxy)-1-(7,8,9,10-tetrahydro-6H-7,10-epiminocyclohepta[b]benzofuran-3-yl)pyridin-2(1H)-one hydrochloride

### c) tert-Butyl 3-(4-((5-fluoropyridin-2-yl)methoxy)-2-oxopyridin-1(2H)-yl)-7,8,9,10-tetrahydro-6H-7,10-epiminocyclohepta[b]benzofuran-11-carboxylate

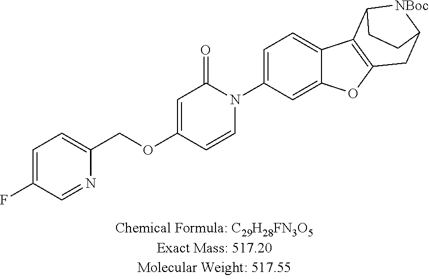 [SMILES: CC(C)(C)OC(=O)N1C2CCC1c1c(oc3cc(-n4ccc(OCc5ccc(F)cn5)cc4=O)ccc13)C2]

tert-Butyl 3-bromo-7,8,9,10-tetrahydro-6H-7,10-epiminocyclohepta[b]benzofuran-11-carboxylate (0.30 g, 0.85 mmol) and 4-((5-fluoropyridin-2-yl)methoxy)pyridin-2(1H)-one (0.18 g, 0.85 mmol) were reacted according to Example 74 (step a) to provide the title compound (85 mg, 20%) as an off-white solid: ¹H NMR (300 MHz, CDCl₃) δ 8.48 (br s, 1H), 7.56-7.40 (m, 5H), 7.29 (d, J=7.5 Hz, 1H), 7.23-7.13 (m, 1H), 6.13-6.03 (m, 1H), 5.24-5.05 (m, 3H), 4.80-4.52 (m, 1H), 3.60-3.30 (m, 1H), 2.56 (d, J=16.5 Hz, 1H), 2.43-2.12 (m, 2H), 2.07-1.92 (m, 1H), 1.73-1.52 (m, 1H), 1.41 (s, 9H); APCI MS m/z 518 [M+H]⁺.

### d) 4-((5-Fluoropyridin-2-yl)methoxy)-1-(7,8,9,10-tetrahydro-6H-7,10-epiminocyclohepta[b]benzofuran-3-yl)pyridin-2(1H)-one hydrochloride

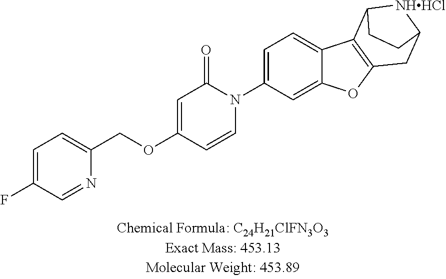

tert-Butyl 3-(4-((5-fluoropyridin-2-yl)methoxy)-2-oxopyridin-1(2H)-yl)-7,8,9,10-tetrahydro-6H-7,10-epiminocyclohepta[b]benzofuran-11-carboxylate (85 mg, 0.21 mmol) was treated with cone. HCl (2 mL) according to Example 74 (step b) to provide the title compound (68 mg, 91%) as an off white solid: ¹H NMR (400 MHz, DMSO-d₆) δ 10.07-9.97 (m, 1H), 9.53 (d, J=10.0 Hz, 1H), 8.62 (d, J=2.8 Hz, 1H), 7.87-7.75 (m, 2H), 7.69-7.59 (m, 3H), 7.27 (dd, J=8.4 Hz, 1.6 Hz, 1H), 6.16 (dd, J=7.6 Hz, 2.4 Hz, 1H), 5.99 (d, J=2.8 Hz, 1H), 5.27-5.19 (m, 3H), 4.53 (m, 1H), 3.48 (dd, J=17.6 Hz, 4.4 Hz, 1H), 3.00 (d, J=16.8 Hz, 1H), 2.37-2.50 (m, 2H), 2.17 (t, J=10.0 Hz, 1H), 1.93-1.80 (m, 1H); APCI MS m/z 418 [M+H]⁺; HPLC (Method C)>99% (AUC), t_{R}=19.59 min.

In accordance with further embodiments, there are provided the following compounds, which may be synthesized by analogy using the methods shown and described above:

TABLE 1
Additional Compounds

| Name | Structure |
|---|---|
| 4-Phenethyl-1-(1,2,3,4-tetrahydrobenzofuro[3,2- c]pyridin-7-yl)pyrimidin-2(1H)-one | 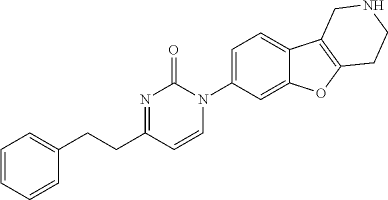 [SMILES: O=c1nc(CCc2ccccc2)ccn1-c1ccc2c3c(oc2c1)CCNC3] |
| 4-(Benzyloxy)-1-(1-methyl-1,2,3,4- tetrahydrobenzothieno[3,2-c]pyridin-7- yl)pyridin-2(1H)-one | 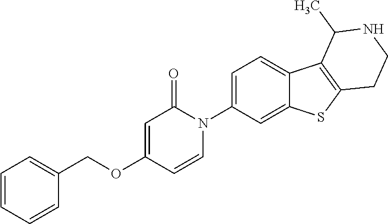 [SMILES: CC1NCCc2sc3cc(-n4ccc(OCc5ccccc5)cc4=O)ccc3c21] |
| 4-(Benzyloxy)-1-(1,1-dimethyl-1,2,3,4- tetrahydrobenzothieno[3,2-c]pyridin-7- yl)pyridin-2(1H)-one | 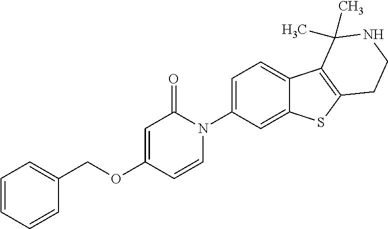 [SMILES: CC1(C)NCCc2sc3cc(-n4ccc(OCc5ccccc5)cc4=O)ccc3c21] |
| 4-((5-Fluoropyridin-2-yl)methoxy)-1-(1-methyl- 1,2,3,4-tetrahydrobenzothieno[3,2-c]pyridin-7- yl)pyridin-2(1H)-one | 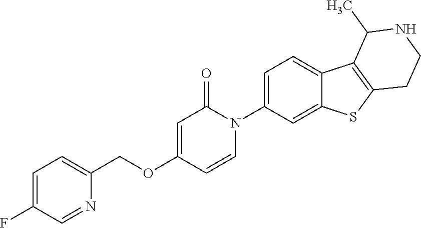 [SMILES: CC1NCCc2sc3cc(-n4ccc(OCc5ccc(F)cn5)cc4=O)ccc3c21] |
| 1-(1,1-Dimethyl-1,2,3,4- tetrahydrobenzothieno[3,2-c]pyridin-7-yl)-4-((5- fluoropyridin-2-yl)methoxy)pyridin-2(1H)-one | 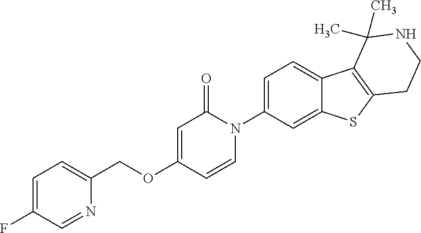 [SMILES: CC1(C)NCCc2sc3cc(-n4ccc(OCc5ccc(F)cn5)cc4=O)ccc3c21] |
| 5-(Benzyloxy)-2-(1,2,3,4- tetrahydrobenzofuro[3,2-c]pyridin-7- yl)pyridazin-3(2H)-one | 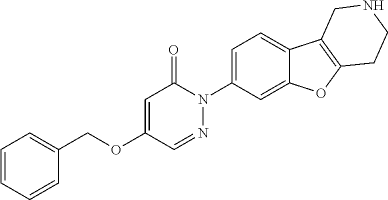 [SMILES: O=c1cc(OCc2ccccc2)cnn1-c1ccc2c3c(oc2c1)CCNC3] |
| 4-Phenethyl-1-(2,3,4,5-tetrahydro-1H- benzofuro[3,2-c]azepin-8-yl)pyrimidin-2(1H)- one | 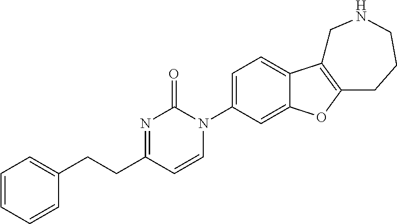 [SMILES: O=c1nc(CCc2ccccc2)ccn1-c1ccc2c3c(oc2c1)CCCNC3] |
| 4-((5-Chloropyridin-2-yl)methoxy)-1-(1,2,3,4- tetrahydrobenzothieno[3,2-c]pyridin-7- yl)pyridin-2(1H)-one | 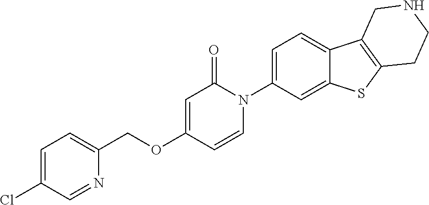 [SMILES: O=c1cc(OCc2ccc(Cl)cn2)ccn1-c1ccc2c3c(sc2c1)CCNC3] |
| 4-((5-Fluoropyridin-2-yl)methoxy)-1-(3-methyl- 1,2,3,4-tetrahydrobenzothieno[3,2-c]pyridin-7- yl)pyridin-2(1H)-one | 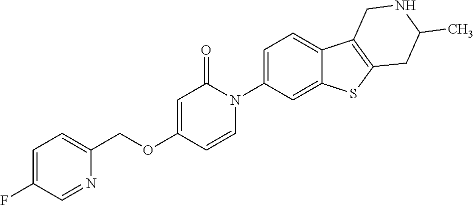 [SMILES: CC1Cc2sc3cc(-n4ccc(OCc5ccc(F)cn5)cc4=O)ccc3c2CN1] |
| 4-(Benzyloxy)-1-(3-methyl-1,2,3,4- tetrahydrobenzothieno[3,2-c]pyridin-7- yl)pyridin-2(1H)-one | 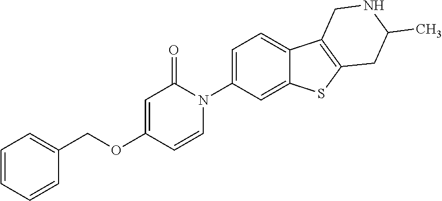 [SMILES: CC1Cc2sc3cc(-n4ccc(OCc5ccccc5)cc4=O)ccc3c2CN1] |
| 4-(Benzyloxy)-1-(1,2,3,4- tetrahydrobenzothieno[3,2-c]pyridin-7- yl)pyridin-2(1H)-one | 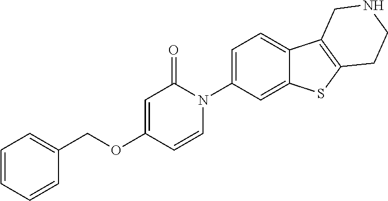 [SMILES: O=c1cc(OCc2ccccc2)ccn1-c1ccc2c3c(sc2c1)CCNC3] |
| 4-(2-(5-Chloropyridin-2-yl)ethyl)-1-(1,2,3,4- tetrahydrobenzothieno[2,3-c]pyridin-7- yl)pyrimidin-2(1H)-one | 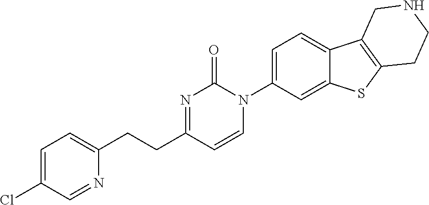 [SMILES: O=c1nc(CCc2ccc(Cl)cn2)ccn1-c1ccc2c3c(sc2c1)CCNC3] |
| 1-(1,1-Dimethyl-1,2,3,4- tetrahydrobenzofuro[3,2-c]pyridin-7-yl)-4-((5- fluoropyridin-2-yl)methoxy)pyridin-2(1H)-one | 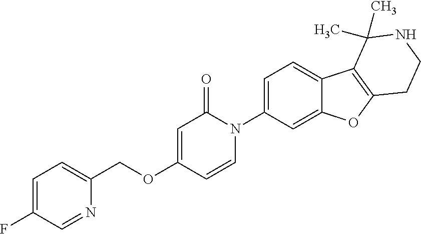 [SMILES: CC1(C)NCCc2oc3cc(-n4ccc(OCc5ccc(F)cn5)cc4=O)ccc3c21] |
| 4-(Benzyloxy)-1-(1,1-dimethyl-1,2,3,4- tetrahydrobenzofuro[3,2-c]pyridin-7-yl)pyridin- 2(1H)-one | 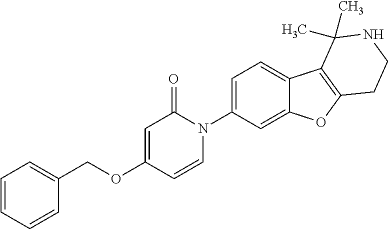 [SMILES: CC1(C)NCCc2oc3cc(-n4ccc(OCc5ccccc5)cc4=O)ccc3c21] |
| 4-(Benzyloxy)-1-(8-fluoro-1,2,3,4- tetrahydrobenzofuro[3,2-c]pyridin-7-yl)pyridin- 2(1H)-one | 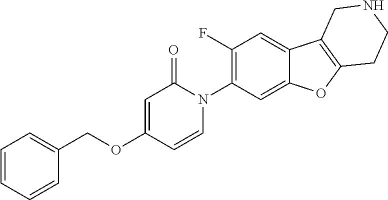 [SMILES: O=c1cc(OCc2ccccc2)ccn1-c1cc2oc3c(c2cc1F)CNCC3] |
| 4-(Benzyloxy)-1-(8-chloro-1,2,3,4- tetrahydrobenzofuro[3,2-c]pyridin-7-yl)pyridin- 2(1H)-one | 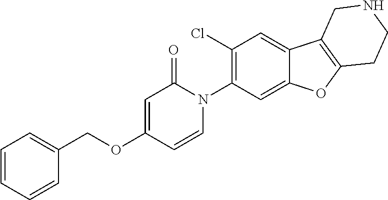 [SMILES: O=c1cc(OCc2ccccc2)ccn1-c1cc2oc3c(c2cc1Cl)CNCC3] |
| 1-(8-Fluoro-1,2,3,4-tetrahydrobenzofuro[3,2- c]pyridin-7-yl)-4-((5-fluoropyridin-2- yl)methoxy)pyridin-2(1H)-one | 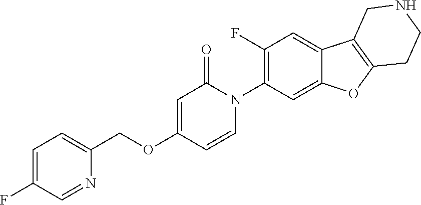 [SMILES: O=c1cc(OCc2ccc(F)cn2)ccn1-c1cc2oc3c(c2cc1F)CNCC3] |
| 1-(8-Chloro-1,2,3,4-tetrahydrobenzofuro[3,2- c]pyridin-7-yl)-4-((5-fluoropyridin-2- yl)methoxy)pyridin-2(1H)-one | 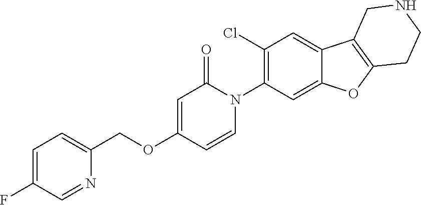 [SMILES: O=c1cc(OCc2ccc(F)cn2)ccn1-c1cc2oc3c(c2cc1Cl)CNCC3] |
| 4-(Benzyloxy)-1-(6-fluoro-1,2,3,4- tetrahydrobenzofuro[3,2-c]pyridin-7-yl)pyridin- 2(1H)-one | 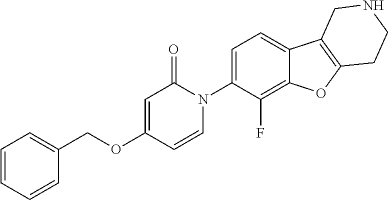 [SMILES: O=c1cc(OCc2ccccc2)ccn1-c1ccc2c3c(oc2c1F)CCNC3] |
| 4-(Benzyloxy)-1-(6-chloro-1,2,3,4- tetrahydrobenzofuro[3,2-c]pyridin-7-yl)pyridin- 2(1H)-one | 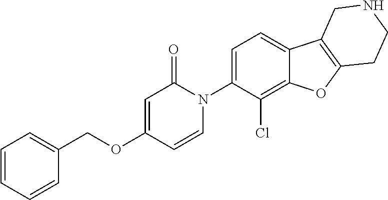 [SMILES: O=c1cc(OCc2ccccc2)ccn1-c1ccc2c3c(oc2c1Cl)CCNC3] |
| 1-(6-Fluoro-1,2,3,4-tetrahydrobenzofuro[3,2- c]pyridin-7-yl)-4-((5-fluoropyridin-2- yl)methoxy)pyridin-2(1H)-one | 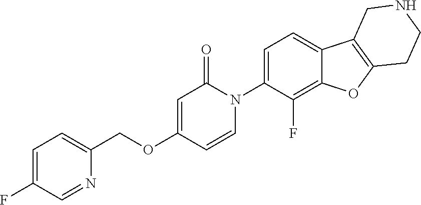 [SMILES: O=c1cc(OCc2ccc(F)cn2)ccn1-c1ccc2c3c(oc2c1F)CCNC3] |
| 1-(6-Chloro-1,2,3,4-tetrahydrobenzofuro[3,2- c]pyridin-7-yl)-4-((5-fluoropyridin-2- yl)methoxy)pyridin-2(1H)-one | 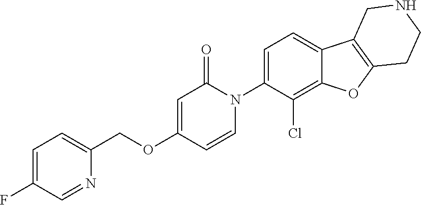 [SMILES: O=c1cc(OCc2ccc(F)cn2)ccn1-c1ccc2c3c(oc2c1Cl)CCNC3] |
| 4-(Benzyloxy)-1-(7,8,9,10-tetrahydro-6H-7,10- epiminobenzo[b]cyclohepta[d]thiophen-3- yl)pyridin-2(1H)-one | 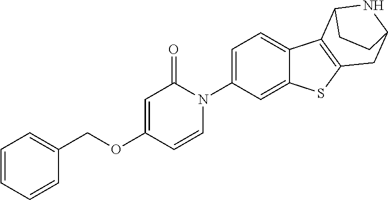 [SMILES: O=c1cc(OCc2ccccc2)ccn1-c1ccc2c3c(sc2c1)CC1CCC3N1] |
| 4-((5-Fluoropyridin-2-yl)methoxy)-1-(7,8,9,10- tetrahydro-6H-7,10- epiminobenzo[b]cyclohepta[d]thiophen-3- yl)pyridin-2(1H)-one | 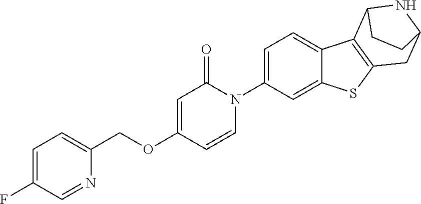 [SMILES: O=c1cc(OCc2ccc(F)cn2)ccn1-c1ccc2c3c(sc2c1)CC1CCC3N1] |
| 4-(Benzyloxy)-1-(6,7,8,9,10,11-hexahydro-7,11- epiminobenzo[b]cycloocta[d]thiophen-3-N yl)pyridin-2(1H)-one | 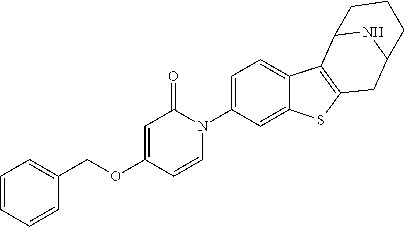 [SMILES: O=c1cc(OCc2ccccc2)ccn1-c1ccc2c3c(sc2c1)CC1CCCC3N1] |
| 4-((5-Fluoropyridin-2-yl)methoxy)-1- (6,7,8,9,10,11-hexahydro-7,11- epiminobenzo[b]cycloocta[d]thiophen-3- yl)pyridin-2(1H)-one | 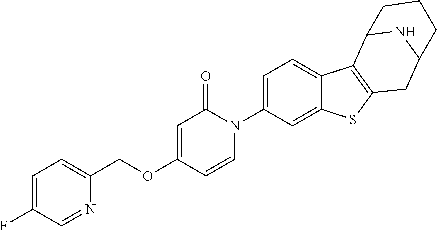 [SMILES: O=c1cc(OCc2ccc(F)cn2)ccn1-c1ccc2c3c(sc2c1)CC1CCCC3N1] |
| 4-(Benzyloxy)-1-(6,7,8,9,10,11-hexahydro-7,11- epiminocycloocta[b]benzofuran-3-yl)pyridin- 2(1H)-one | 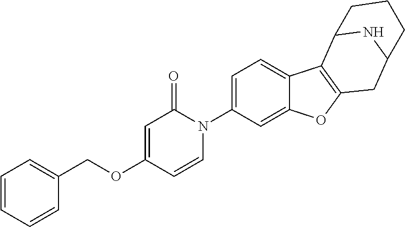 [SMILES: O=c1cc(OCc2ccccc2)ccn1-c1ccc2c3c(oc2c1)CC1CCCC3N1] |
| 4-((5-Fluoropyridin-2-yl)methoxy)-1- (6,7,8,9,10,11-hexahydro-7,11 epiminocycloocta[b]benzofuran-3-yl)pyridin- 2(1H)-one | 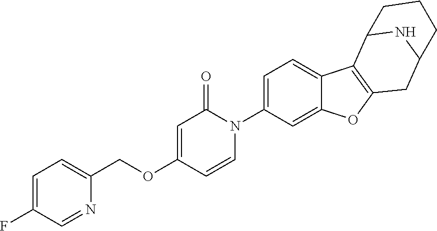 [SMILES: O=c1cc(OCc2ccc(F)cn2)ccn1-c1ccc2c3c(oc2c1)CC1CCCC3N1] |

### Example 130

### Binding Assay for Human Melanin-Concentrating Hormone (MCH-1) Receptor

Evaluation of the affinity of compounds for the human MCH-1 receptor was accomplished using 4-(3,4,5-tritritiumbenzyloxy)-1-(1-(2-(pyrrolidin-1-yl)ethyl)-1H-indazol-5-yl)pyridin-2(1H)-one and membranes prepared from stable CHO-K1 cells expressing the human MCH-1 receptor obtained from Euroscreen (Batch 1138). Cell membrane homogenates (8.92 g protein) were incubated for 60 min at 25° C. with 1.4 nM of the [³H]-labeled compound in the absence or presence of the test compound in 50 mM Tris-HCl buffer, pH 7.4. Nonspecific binding was determined in the presence of 50 μM 1-(5-(4-cyanophenyl)bicyclo[3.1.0]hexan-2-yl)-3-(4-fluoro-3-(trifluoromethyl)phenyl)-1-(3-(4-methylpiperazin-1-yl)propyl)urea. Following incubation, the samples were filtered rapidly under vacuum through Skatron 11731 filters, pre-soaked in 0.5% polyethylenimine, and washed with ice-cold 50 mM Tris-HCl buffer, pH 7.4, (wash setting 9,9,0) using a Skatron cell harvester. The filters were counted for radioactivity in a liquid scintillation counter (Tri-Carb 2100TR, Packard) using a scintillation cocktail (Ultima Gold MV, Perkin Elmer).

The results are expressed as a percent inhibition of the control radioligand specific binding. The IC₅₀ value (concentration causing a half-maximal inhibition of control specific binding) and Hill coefficient (n_{H}) were determined by non-linear regression analysis of the competition curve using Hill equation curve fitting. The inhibition constant (Kᵢ) was calculated from the Cheng Prusoff equation: (Kᵢ=IC₅₀/(1+(L/K_{D})), where L=concentration of radioligand in the assay, and K_{D}=affinity of the radioligand for the receptor.

By methods as described above, the compounds listed in Table 2 were synthesized and tested for biological activity.

TABLE 2
Compounds Tested for Biological Activity

| Ex, | | MCH₁ | Mass | |
|---|---|---|---|---|
| No. | Structure | Kᵢ (nM) | Spec | ¹H NMR Data |
| 2 | 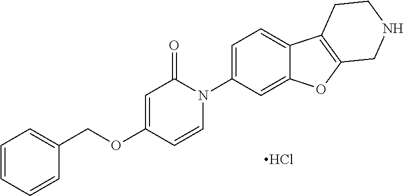 [SMILES: O=c1cc(OCc2ccccc2)ccn1-c1ccc2c3c(oc2c1)CNCC3 ; Cl] | 23 | 373 | ¹H NMR (500 MHz, DMSO-d₆) δ 9.83 (br s, 2H), 7.70-7.68 (m, 2H), 7.60 (d, J = 6.5 Hz, 1H), 7.48-7.40 (m, 4H), 7.39-7.36 (m, 1H), 7.30-7.27 (m, 1H), 6.12 (dd, J = 7.5, 2.5 Hz, 1H), 5.99 (d, J = 2.5 Hz, 1H), 5.15 (s, 2H), 4.43 (s, 2H), 3.46 (t, J = 5.5 Hz, 2H), 2.96 (t, J = 5.5 Hz, 2H) |
| 3 | 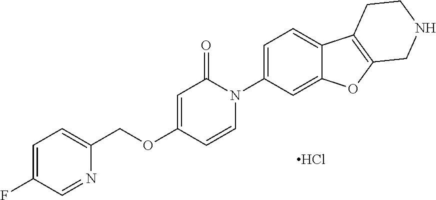 [SMILES: O=c1cc(OCc2ccc(F)cn2)ccn1-c1ccc2c3c(oc2c1)CNCC3 ; Cl] | 59 | 392 | ¹H NMR (500 MHz, DMSO-d₆) δ 9.58 (br s, 2H), 8.62 (d, J = 2.5 Hz, 1H), 7.85-7.80 (m, 1H), 7.71-7.68 (m, 2H), 7.67-7.64 (m, 1H), 7.63-7.60 (m, 1H), 7.28 (dd, J = 8.5, 2.0 Hz, 1H), 6.16 (dd, J = 7.5, 2.5 Hz, 1H), 5.99 (d, J = 2.5 Hz, 1H), 5.22 (s, 2H), 4.44 (s, 2H), 3.47 (t, J = 6.0 Hz, 2H), 2.96 (t, J = 5.5 Hz, 2H) |
| 4 | 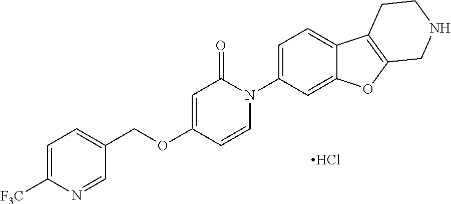 [SMILES: O=c1cc(OCc2ccc(C(F)(F)F)nc2)ccn1-c1ccc2c3c(oc2c1)CNCC3 ; Cl] | 1307 | 412 | ¹H NMR (500 MHz, DMSO-d₆) δ 9.81 (br s, 2H), 9.20 (d, J = 2.0 Hz, 1H), 8.50 (dd, J = 8.5, 2.0 Hz, 1H), 8.05 (d, J = 8.0 Hz, 1H), 7.89 (d, J = 7.0 Hz, 1H), 7.82 (d, J = 1.5 Hz, 1H), 7.75 (d, J = 8.5 Hz, 1H), 7.39 (dd, J = 8.0, 2.0 Hz, 1H), 7.03 (d, J = 2.0 Hz, 1H), 6.83 (dd, J = 7.5, 2.5 Hz, 1H), 4.45 (s, 2H), 3.48 (t, J = 6.0 Hz, 2H), 3.02-2.97 (m, 2H) |
| 5 | 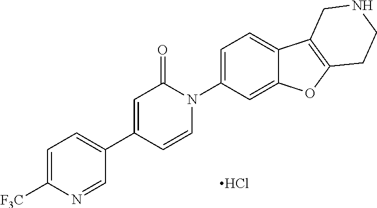 [SMILES: O=c1cc(-c2ccc(C(F)(F)F)nc2)ccn1-c1ccc2c3c(oc2c1)CCNC3 ; Cl] | 56 | 412 | ¹H NMR (500 MHz, DMSO-d₆) δ 9.62 (br s, 2H), 9.20 (d, J = 2.0 Hz, 1H), 8.51 (dd, J = 8.0, 2.0 Hz, 1H), 8.05 (d, J = 8.0 Hz, 1H), 7.88 (d, J = 7.5 Hz, 1H), 7.80 (d, J = 2.0 Hz, 1H), 7.74 (d, J = 8.5 Hz, 1H), 7.38 (dd, J = 8.5, 2.0 Hz, 1H), 7.03 (d, J = 2.0 Hz, 1H), 6.83 (dd, J = 7.5, 2.0 Hz, 1H), 4.37 (s, 2H), 3.56 (t, J = 6.0 Hz, 2H), 3.16-3.12 (m, 2H) |
| 6 | 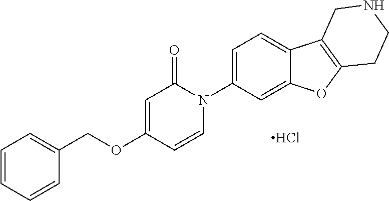 [SMILES: O=c1cc(OCc2ccccc2)ccn1-c1ccc2c3c(oc2c1)CCNC3 ; Cl] | 3.3 | 373 | ¹H NMR (500 MHz, DMSO-d₆) δ 9.47 (br s, 2H), 7.70-7.67 (m, 2H), 7.59 (d, J = 7.5 Hz, 1H), 7.48-7.47 (m, 2H), 7.46-7.41 (m, 2H), 7.39-7.35 (m, 1H), 7.27 (dd, J = 8.0, 2.0 Hz, 1H), 6.12 (dd, J = 7.5, 2.5 Hz, 1H), 5.99 (d, J = 3.0 Hz, 1H), 5.15 (s, 2H), 4.35 (s, 2H), 3.59-3.52 (m, 2H), 3.11 (t, J = 5.5 Hz. 2H) |
| 7 | 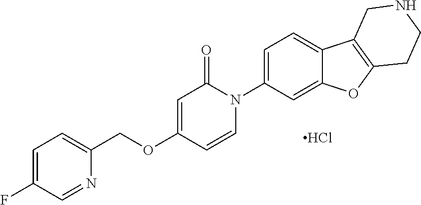 [SMILES: O=c1cc(OCc2ccc(F)cn2)ccn1-c1ccc2c3c(oc2c1)CCNC3 ; Cl] | 7.3 | 392 | ¹H NMR (500 MHz, DMSO-d₆) δ 8.61 (d, J = 2.5 Hz, 1H), 7.84-7.80 (m, 1H), 7.66-7.64 (m, 1H), 7.61-7.54 (m, 3H), 7.19 (dd, J = 8.0, 2.0 Hz, 1H), 6.14 (dd, J = 7.5, 2.5 Hz, 1H), 5.98 (d, J = 2.5 Hz, 1H), 5.21 (s, 2H), 3.97 (s, 2H), 3.18 (t, J = 5.5 Hz, 2H), 2.81 (t, J = 5.5 Hz, 2H) |
| 8 | 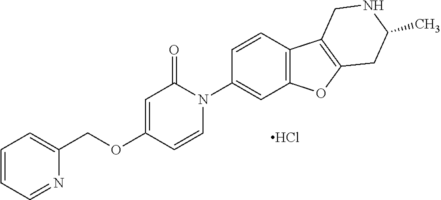 [SMILES: C[C@@H]1Cc2oc3cc(-n4ccc(OCc5ccccn5)cc4=O)ccc3c2CN1 ; Cl] | 57 | 388 | ¹H NMR (500 MHz, DMSO-d₆) δ 9.66 (br s, 1H), 9.47 (br s, 1H), 8.62 (s, 1H), 7.91 (t, J = 7.7 Hz, 1H), 7.68 (m, 2H), 7.61 (d, J = 7.6 Hz, 1H), 7.58 (d, J = 7.8 Hz, 1H), 7.42 (m, 1H), 7.27 (d, J = 8.25 Hz, 1H), 6.17 (d, J = 7.6 Hz, 1H), 5.97 (s, 1H), 5.23 (s, 2H), 4.34 (m, 2H), 3.78 (m, 1H), 3.22 (dd, J = 17.3, 12.8 Hz, 1H), 2.90 (dd, J = 17.3, 7.9 Hz, 1H), 1.46 (d, J = 6.5 Hz, 3H) |
| 9 | 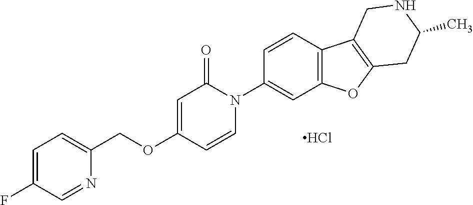 [SMILES: C[C@@H]1Cc2oc3cc(-n4ccc(OCc5ccc(F)cn5)cc4=O)ccc3c2CN1 ; Cl] | 10 | 406 | ¹H NMR (500 MHz, DMSO-d₆) δ 9.33 (br s, 2H), 8.62 (d, J = 2.95 Hz, 1H), 7.82 (t, J = 8.7 Hz, 1H), 7.66 (m, 4H), 7.27 (d, J = 8.3 Hz, 1H), 6.16 (dd, J = 7.6, 4.9 Hz, 1H), 5.99 (s, 1H), 5.21 (s, 2H), 4.41 (m, 2H), 3.78 (m, 1H), 3.22 (dd, J = 17.2, 4.6 Hz, 1H), 2.88 (dd, J = 17.3, 7.85 Hz, 1H), 1.44 (d, J = 6.5 Hz, 3H) |
| 10 | 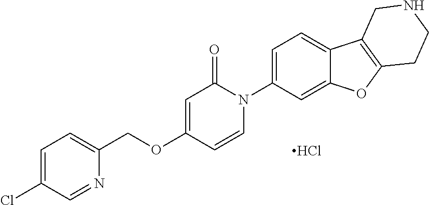 [SMILES: O=c1cc(OCc2ccc(Cl)cn2)ccn1-c1ccc2c3c(oc2c1)CCNC3 ; Cl] | 3.3 | 408 | ¹H NMR (500 MHz, DMSO-d₆) δ 9.50 (s, 2H), 8.67 (d, J = 2.5 Hz, 1H), 8.03 (dd, J = 8.5, 2.5 Hz, 1H), 7.70-7.68 (m, 2H), 7.61 (t, J = 7.5 Hz, 2H), 7.27 (dd, J = 8.5, 2.0 Hz, 1H), 6.17 (dd, J = 7.5, 2.5 Hz, 1H), 5.97 (d, J = 3.0 Hz, 1H), 5.24 (s, 2H), 4.36 (s, 2H), 3.55 (d, J = 4.0 Hz, 2H), 3.11 (t, J = 6.0 Hz, 2H) |
| 11 | 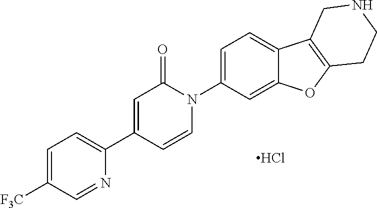 [SMILES: O=c1cc(-c2ccc(C(F)(F)F)cn2)ccn1-c1ccc2c3c(oc2c1)CCNC3 ; Cl] | 166 | 412 | ¹H NMR (500 MHz, DMSO-d₆) δ 9.54 (s, 2H), 9.15 (t, J = 1.0 Hz, 1H), 8.39- 8.36 (m, 2H), 7.87 (d, J = 7.5 Hz, 1H), 7.82 (d, J = 1.5 Hz, 1H), 7.75 (d, J = 8.5 Hz, 1H), 7.39 (dd, J = 8.5, 2.0 Hz, 1H), 7.30 (d, J = 2.0 Hz, 1H), 7.09 (dd, J = 7.5, 2.0 Hz, 1H), 4.38 (s, 2H), 3.58-3.56 (m, 2H), 3.15-3.13 (m, 2H) |
| 12 | 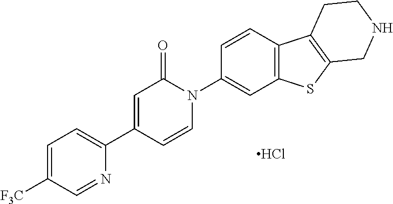 [SMILES: O=c1cc(-c2ccc(C(F)(F)F)cn2)ccn1-c1ccc2c3c(sc2c1)CNCC3 ; Cl] | 93 | 428 | ¹H NMR (500 MHz, CD₃OD) δ 9.05 (s, 1H), 8.29-8.27 (dd, J = 8.4, 2.1 Hz, 1H), 8.22 (d, J = 8.4 Hz, 1H), 8.07 (d, J = 1.8 Hz, 1H), 7.91 (d, J = 8.5 Hz, 1H), 7.83 (d, J = 7.0 Hz, 1H), 7.55- 7.53 (dd, J = 8.5, 1.9 Hz, 1H), 7.39 (d, J = 1.5 Hz, 1H), 7.27-7.25 (dd, J = 7.2, 2.0 Hz, 1H), 4.59 (s, 2H), 3.69 (t, J = 6.5 Hz, 2H), 3.23 (t, J = 6.5 Hz, 2H) |
| 13 | 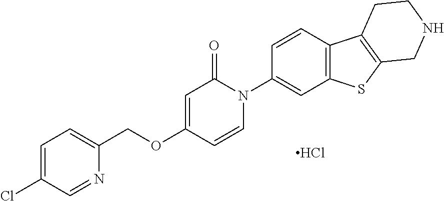 [SMILES: O=c1cc(OCc2ccc(Cl)cn2)ccn1-c1ccc2c3c(sc2c1)CNCC3 ; Cl] | 3.1 | 424 | ¹H NMR (500 MHz, DMSO-d₆) δ 9.66 (s, 2H), 8.67 (d, J = 2.4 Hz, 1H), 8.05 (d, J = 1.8 Hz, 1H), 8.06-8.02 (dd, J = 8.3, 2.5 Hz, 1H), 7.92 (d, J = 8.4 Hz, 1H), 7.65 (d, J = 8.0 Hz, 1H), 7.61 (d, J = 8.0 Hz, 1H), 7.42-7.40 (dd, J = 8.5, 1.9 Hz, 1H), 6.18-6.16 (dd, J = 7.6, 2.7 Hz, 1H), 5.99 (d, J = 2.7 Hz, 1H), 5.23 (s, 2H), 4.49 (s, 2H), 3.52-3.51 (m, 2H), 3.08 (t, J = 6.2 Hz, 2H) |
| 14 | 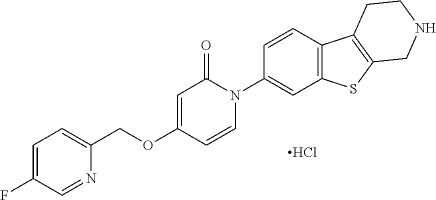 [SMILES: O=c1cc(OCc2ccc(F)cn2)ccn1-c1ccc2c3c(sc2c1)CNCC3 ; Cl] | 6.2 | 408 | ¹H NMR (500 MHz, DMSO-d₆) δ 9.84 (s, 2H), 8.62 (d, J = 2.9 Hz, 1H), 8.05 (d, J = 1.8 Hz, 1H), 7.84-7.80 (m, 2H), 7.67-7.63 (m, 2H), 7.42- 7.39 (dd, J = 8.5, 1.9 Hz, 1H), 6.17- 6.15 (dd, J = 7.6, 2.8 Hz, 1H), 6.01 (d, J = 2.7 Hz, 1H), 5.22 (s, 2H), 4.50- 4.46 (m, 2H), 3.50-3.47 (m, 2H), 3.08 (t, J = 6.5 Hz, 2H) |
| 15 | 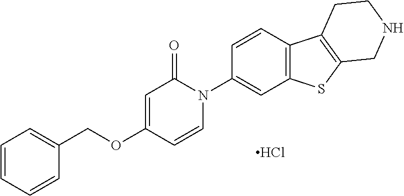 [SMILES: O=c1cc(OCc2ccccc2)ccn1-c1ccc2c3c(sc2c1)CNCC3 ; Cl] | 4.0 | 389 | ¹H NMR (500 MHz, DMSO-d₆) δ 9.76 (s, 2H), 8.05 (d, J = 1.7 Hz, 1H), 7.83 (d, J = 8.4 Hz, 1H), 7.62 (d, J = 7.6 Hz, 1H), 7.47-7.36 (m, 6H), 6.14- 6.12 (dd, J = 7.6, 2.6 Hz, 1H), 6.00 (d, J = 2.6 Hz, 1H), 5.15 (s, 2H), 4.50- 4.43 (m, 2H), 3.53-3.47 (m, 2H), 3.11-3.05 (m, 2H) |
| 16 | 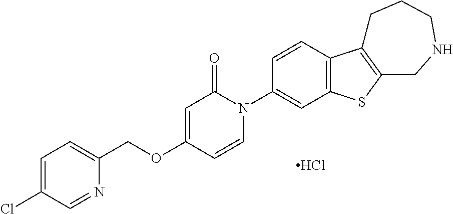 [SMILES: O=c1cc(OCc2ccc(Cl)cn2)ccn1-c1ccc2c3c(sc2c1)CNCCC3 ; Cl] | 5.0 | 438 | ¹H NMR (500 MHz, DMSO-d₆) δ 9.46 (s, 2H), 8.68 (d, J = 2.0 Hz, 1H), 8.05-8.01 (m, 2H), 7.92 (d, J = 8.6 Hz, 1H), 7.65 (d, J = 7.7 Hz, 1H), 7.61 (d, J = 8.1 Hz, 1H), 7.42-7.39 (dd, J = 8.6, 1.9 Hz, 1H), 6.19-6.16 (dd, J = 7.6, 2.7 Hz, 1H), 5.99 (d, J = 2.7 Hz, 1H), 5.24 (s, 2H), 4.61-4.52 (m, 2H), 3.54-3.42 (m, 2H), 3.18-3.08 (m, 2H), 2.05-1.95 (m, 2H) |
| 17 | 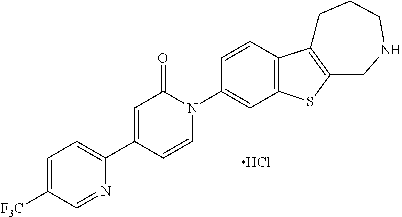 [SMILES: O=c1cc(-c2ccc(C(F)(F)F)cn2)ccn1-c1ccc2c3c(sc2c1)CNCCC3 ; Cl] | 927 | 442 | ¹H NMR (500 MHz, DMSO-d₆) δ 9.35 (s, 2H), 9.15 (s, 1H), 8.45-8.31 (m, 2H), 8.17 (d, J = 1.8 Hz, 1H), 7.97 (d, J = 8.7 Hz, 1H), 7.90 (d, J = 7.2 Hz, 1H), 7.54-7.51 (dd, J = 8.6, 1.9 Hz, 1H), 7.32 (d, J = 1.6 Hz, 1H), 7.12-7.09 (dd, J = 7.2, 1.9 Hz, 1H), 4.61 (s, 2H), 3.56-3.45 (m, 2H), 3.22- 3.20 (m, 2H), 2.08-1.92 (m, 2H) |
| 18 | 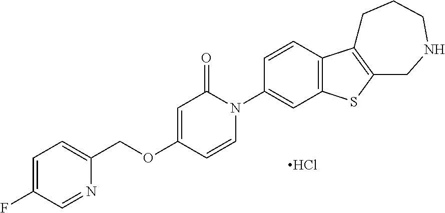 [SMILES: O=c1cc(OCc2ccc(F)cn2)ccn1-c1ccc2c3c(sc2c1)CNCCC3 ; Cl] | 12 | 422 | ¹H NMR (500 MHz, DMSO-d₆) δ 9.42 (s, 2H), 8.62 (d, J = 2.9 Hz, 1H), 8.03 (d, J = 1.9 Hz, 1H), 7.91 (d, J = 8.6 Hz, 1H), 7.84-7.81 (td, J = 8.7, 2.9 Hz, 1H), 7.67-7.63 (m, 2H), 7.41- 7.39 (dd, J = 8.6, 1.9 Hz, 1H), 6.17- 6.15 (dd, J = 7.6, 2.7 Hz, 1H), 6.00 (d, J = 2.8 Hz, 1H), 5.22 (s, 2H), 4.60- 4.56 (m, 2H), 3.51-3.45 (m, 2H), 3.16-3.11 (m, 2H), 2.03-1.97 (m, 2H) |
| 19 | 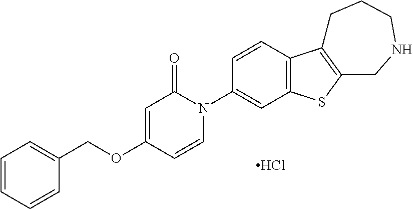 [SMILES: O=c1cc(OCc2ccccc2)ccn1-c1ccc2c3c(sc2c1)CNCCC3 ; Cl] | 8.2 | 403 | ¹H NMR (500 MHz, DMSO-d₆) δ 9.31 (s, 2H), 8.03 (d, J = 1.9 Hz, 1H), 7.91 (d, J = 8.6 Hz, 1H), 7.62 (d, J = 7.6 Hz, 1H), 7.48-7.36 (m, 6H), 6.14- 6.12 (dd, J = 7.6, 2.7 Hz, 1H), 6.00 (d, J = 2.7 Hz, 1H), 5.15 (s, 2H), 4.60- 4.56 (m, 2H), 3.51-3.46 (m, 2H), 3.14-3.12 (m, 2H), 2.00-1.97 (m, 2H) |
| 20 | 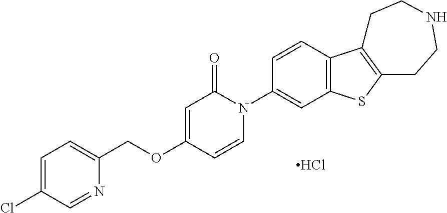 [SMILES: O=c1cc(OCc2ccc(Cl)cn2)ccn1-c1ccc2c3c(sc2c1)CCNCC3 ; Cl] | 3.8 | 438 | ¹H NMR (500 MHz, DMSO-d₆) δ 9.50 (s, 2H), 8.68 (d, J = 2.5 Hz, 1H), 8.05-8.01 (dd, J = 8.4, 2.5 Hz, 1H), 7.97 (d, J = 1.8 Hz, 1H), 7.84 (d, J = 8.6 Hz, 1H), 7.66-7.58 (m, 2H), 7.39- 7.36 (dd, J = 8.6, 2.0 Hz, 1H), 6.18- 6.15 (dd, J = 7.6, 2.7 Hz, 1H), 5.98 (d, J = 2.7 Hz, 1H), 5.23 (s, 2H), 3.43- 3.24 (m, 8H) |
| 21 | 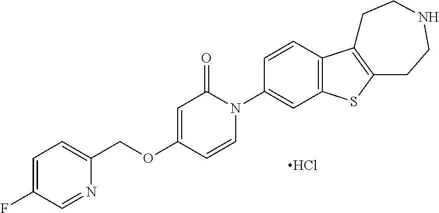 [SMILES: O=c1cc(OCc2ccc(F)cn2)ccn1-c1ccc2c3c(sc2c1)CCNCC3 ; Cl] | 3.8 | 422 | ¹H NMR (500 MHz, DMSO-d₆) δ 9.39 (s, 2H), 8.62 (d, J = 2.9 Hz, 1H), 7.97 (d, J = 1.9 Hz, 1H), 7.86-7.79 (m, 2H), 7.68-7.61 (m, 2H), 7.38- 7.25 (dd, J = 8.5, 1.9 Hz, 1H), 6.17- 6.14 (dd, J = 7.6, 2.7 Hz, 1H), 5.99 (d, J = 2.6 Hz, 1H), 5.22 (s, 2H), 3.39- 3.24 (m, 8H) |
| 22 | 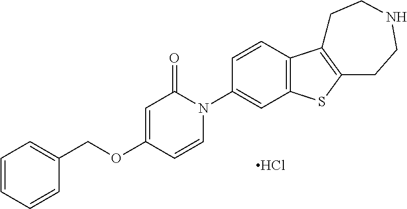 [SMILES: O=c1cc(OCc2ccccc2)ccn1-c1ccc2c3c(sc2c1)CCNCC3 ; Cl] | 4.4 | 403 | ¹H NMR (500 MHz, DMSO-d₆) δ 9.43 (s, 2H), 7.97 (d, J = 1.9 Hz, 1H), 7.82 (d, J = 8.0 Hz, 1H), 7.61 (d, J = 7.6 Hz, 1H), 7.50-7.33 (m, 6H), 6.14- 6.11 (dd, J = 7.6, 2.7 Hz, 1H), 5.99 (d, J = 2.6 Hz, 1H), 5.15 (s, 2H), 3.40 3.22 (m, 8H) |
| 23 | 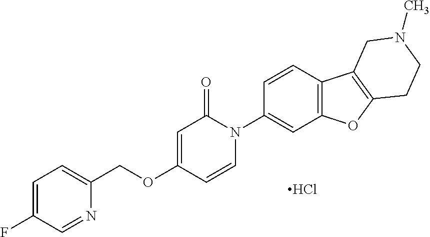 [SMILES: CN1CCc2oc3cc(-n4ccc(OCc5ccc(F)cn5)cc4=O)ccc3c2C1 ; Cl] | 11 | 406 | ¹H NMR (300 MHz, CD₃OD) δ 8.59 (dt, J = 2.8, 0.8 Hz, 1H), 7.90-7.60 (m, 5H), 7.31 (dd, J = 8.3, 1.9 Hz, 1H), 6.40 (dd, J = 7.6, 2.7 Hz, 1H), 6.19 (d, J = 2.7 Hz, 1H), 5.32 (s, 2H), 4.75 (d, J = 14.9 Hz, 1H), 4.41 (d, J = 15.0 Hz, 1H), 3.91-3.89 (m, 1H), 3.76-3.60 (m, 1H), 3.30-3.20 (m, 2H), 3.16 (s, 3H) |
| 24 | 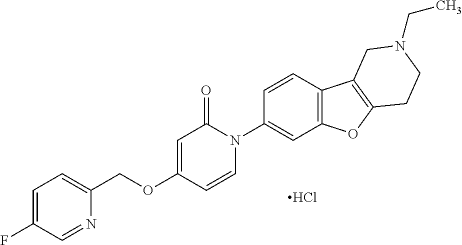 [SMILES: CCN1CCc2oc3cc(-n4ccc(OCc5ccc(F)cn5)cc4=O)ccc3c2C1 ; Cl] | 13 | 420 | ¹H NMR (300 MHz, CD₃OD) δ 8.65- 8.57 (m, 1H), 7.92-7.60 (m, 5H), 7.36-7.26 (m, 1H), 6.42 (dd, J = 7.6, 2.7 Hz, 1H), 6.20 (d, J = 2.8 Hz, 1H), 5.33 (s, 2H), 4.88-4.72 (m, 1H), 4.39 (d, J = 15.3 Hz, 1H), 4.02-3.92 (m, 1H), 3.64-3.62 (m, 1H), 3.56-3.43 (m, 2H), 3.30-3.24 (m, 2H), 1.56- 1.50 (t, J = 7.3 Hz, 3H) |
| 25 | 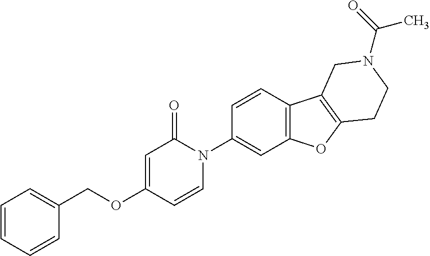 [SMILES: CC(=O)N1CCc2oc3cc(-n4ccc(OCc5ccccc5)cc4=O)ccc3c2C1] | 23 | 415 | ¹H NMR (300 MHz, CDCl₃) δ 7.50- 7.34 (m, 6H), 7.29-7.16 (m, 3H), 6.09-6.05 (m, 2H), 5.05 (s, 2H), 4.74 (d, J = 2.1 Hz, 1H), 4.60 (t, J = 2.0 Hz, 1H), 4.02 (t, J = 5.8 Hz, 1H), 3.84 (t, J = 5.7 Hz, 1H), 3.01-2.83 (m, 2H), 2.24 (s, 1.5H), 2.23 (s, 1.5H) |
| 26 | 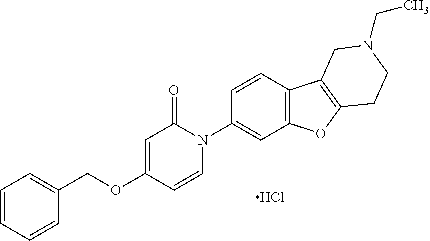 [SMILES: CCN1CCc2oc3cc(-n4ccc(OCc5ccccc5)cc4=O)ccc3c2C1 ; Cl] | 4.9 | 401 | ¹H NMR (300 MHz, CD₃OD) δ 7.74- 7.59 (m, 3H), 7.52-7.26 (m, 6H), 6.38 (dd, J = 7.6, 2.7 Hz, 1H), 6.18 (d, J = 2.7 Hz, 1H), 5.21 (s, 2H), 4.77 (d, J = 15.0 Hz, 1H) 4.39 (d, J = 15.0 Hz, 1H), 4.00-3.90 (m, 1H), 3.72-3.56 (m, 1H), 3.53-3.41 (m, 2H), 3.30- 3.25 (m, 2H), 1.50 (t, J = 7.3 Hz, 3H) |
| 27 | 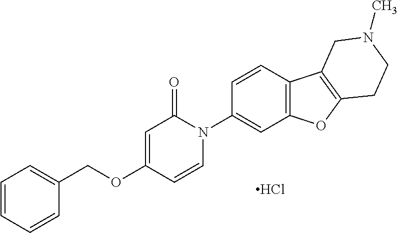 [SMILES: CN1CCc2oc3cc(-n4ccc(OCc5ccccc5)cc4=O)ccc3c2C1 ; Cl] | 8.1 | 387 | ¹H NMR (300 MHz, CD₃OD) δ 7.73- 7.62 (m, 3H), 7.52-7.27 (m, 6H), 6.44 (dd, J = 7.6, 2.7 Hz, 1H), 6.23 (d, J = 2.7 Hz, 1H), 5.23 (s, 2H), 4.75 (d, J = 14.8 Hz, 1H), 4.48-4.35 (m, 1H), 3.97-3.88 (m, 1H), 3.76-3.60 (m, 1H), 3.30-3.25 (m, 2H), 3.16 (s, 3H) |
| 28 | 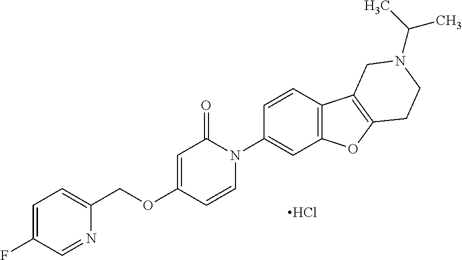 [SMILES: CC(C)N1CCc2oc3cc(-n4ccc(OCc5ccc(F)cn5)cc4=O)ccc3c2C1 ; Cl] | 9.8 | 434 | ¹H NMR (300 MHz, CD₃OD) δ 8.56 (dt, J = 2.9, 0.8 Hz, 1H), 7.86-7.59 (m, 5H), 7.31 (dd, J = 8.3, 1.8 Hz, 1H), 6.38 (dd, J = 7.6, 2.7 Hz, 1H), 6.17 (d, J = 2.7 Hz, 1H), 5.30 (s, 2H), 4.69-4.46 (m, 2H), 3.97-3.80 (m, 2H), 3.65 (td, J = 11.5, 5.6 Hz, 1H), 3.30-3.20 (m, 2H), 1.55-1.50 (m, 6H) |
| 29 | 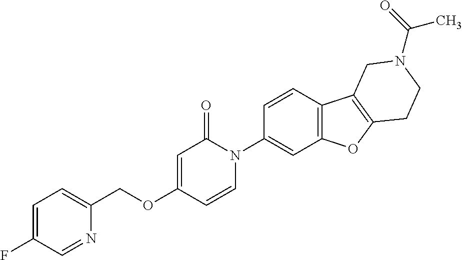 [SMILES: CC(=O)N1CCc2oc3cc(-n4ccc(OCc5ccc(F)cn5)cc4=O)ccc3c2C1] | 46 | 434 | ¹H NMR (300 MHz, CDCl₃) δ 8.53 8.45 (m, 1H), 7.56-7.40 (m, 4H), 7.37-7.14 (m, 2H), 6.16-5.99 (m, 2H), 5.17 (2 × s, 2H), 4.79-4.71 (m, 1.2H), 4.61 (t, J = 2.0 Hz, 0.8H), 4.03 (t, J = 5.8 Hz, 0.8H), 3.85 (t, J = 5.7 Hz, 1.2H). 3.01-2.83 (m, 2H), 2.24 (m, 3H) |
| 30 | 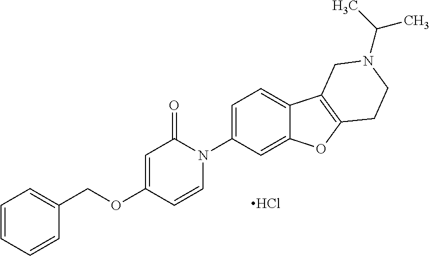 [SMILES: CC(C)N1CCc2oc3cc(-n4ccc(OCc5ccccc5)cc4=O)ccc3c2C1 ; Cl] | 9 | 415 | ¹H NMR (300 MHz, CD₃OD) δ 7.77- 7.62 (m, 3H), 7.53-7.28 (m, 6H), 6.47 (dd, J = 7.5, 2.6 Hz, 1H), 6.25 (d, J = 2.7 Hz, 1H), 5.24 (s, 2H), 4.63 (d, J = 14.7 Hz, 1H), 4.52 (d, J = 14.8 Hz, 1H), 3.96-3.81 (m, 2H), 3.70-3.60 (m, 1H) 3.30-3.24 (m, 2H), 1.52 (m, 6H) |
| 31 | 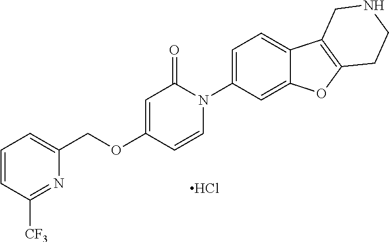 [SMILES: O=c1cc(OCc2cccc(C(F)(F)F)n2)ccn1-c1ccc2c3c(oc2c1)CCNC3 ; Cl] | 23 | 442 | ¹H NMR (300 MHz, CD₃OD) δ 8.15 (t, J = 7.8 Hz, 1H), 8.00 (d, J = 7.5 Hz, 1H), 7.90 (d, J = 8.1 Hz, 1H), 7.83 (d, J = 7.9 Hz, 1H), 7.79-7.68 (m, 2H), 7.39 (dd, J = 8.3, 1.9 Hz, 1H), 6.85 (dd, J = 7.5, 2.6 Hz, 1H), 6.55 (d, J = 2.6 Hz, 1H), 5.50 (s, 2H), 4.50 (d, J = 1.7 Hz, 2H), 3.72 (t, J = 6.1 Hz, 2H), 3.31-3.18 (m, 2H) |
| 32 | 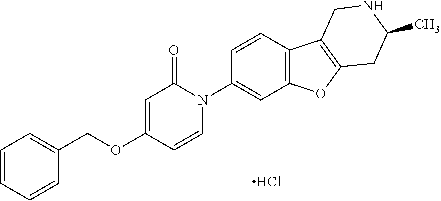 [SMILES: C[C@H]1Cc2oc3cc(-n4ccc(OCc5ccccc5)cc4=O)ccc3c2CN1 ; Cl] | 7.4 | 387 | ¹H NMR (500 MHz, DMSO-d₆) δ 9.57 (br s, 1H), 9.39 (br s, 1H), 7.68 (m, 2H), 7.59 (d, J = 7.65 Hz, 1H), 7.42 (m, 5H), 7.26 (dd, J = 8.2 Hz, 6.6 Hz, 1H), 6.12 (dd, J = 7.6 Hz, 5.0 Hz, 1H), 5.98 (s, 1H), 5.15 (s, 2H), 4.42 (m, 2H), 3.79 (br s, 1H), 3.22 (dd, J = 17.3 Hz, 13.2 Hz, 1H), 2.90 (dd, J = 17.2 Hz, 7.9 Hz, 1H), 1.45 (d, J = 6.5 Hz, 3H) |
| 33 | 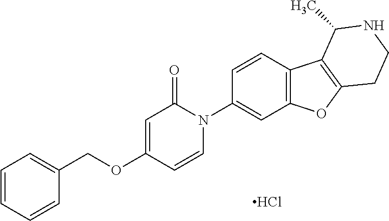 [SMILES: C[C@@H]1NCCc2oc3cc(-n4ccc(OCc5ccccc5)cc4=O)ccc3c21 ; Cl] | 6 | 387 | ¹H NMR (500 MHz, DMSO-d₆) δ 9.65 (br s, 1H), 9.24 (br s, 1H), 7.74 (d, J = 8.3 Hz, 1H), 7.68 (s, 1H), 7.59 (d, J = 7.6 Hz, 1H), 7.43 (m, 5H), 7.26 (dd, J = 8.3 Hz, 6.6 Hz, 1H), 6.12 (dd, J = 7.6 Hz, 5.0 Hz, 1H), 5.98 (s, 1H), 5.15 (s, 2H), 4.80 (bs, 1H), 3.64 (m, 1H), 3.47 (m, 1H), 3.11 (m, 2H), 1.68 (d, J = 6.7 Hz, 3H) |
| 34 | 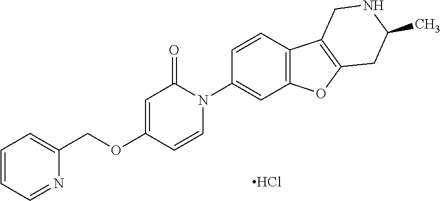 [SMILES: C[C@H]1Cc2oc3cc(-n4ccc(OCc5ccccn5)cc4=O)ccc3c2CN1 ; Cl] | 38 | 388 | ¹H NMR (500 MHz, DMSO-d₆) δ 9.62 (br s, 1H), 9.42 (br s, 1H), 8.63 (m, 1H), 7.92 (t, J = 7.7 Hz, 1H), 7.69 (m, 2H), 7.62 (m, 2H), 7.43 (t, J = 6.7 Hz, 1H), 7.26 (m, 1H), 6.17 (m, 1H), 5.98 (s, 1H), 5.24 (s, 2H), 4.38 (m, 2H), 3.79 (m, 1H), 3.22 (dd, J = 17.4 Hz, 12.7 Hz, 1H), 2.90 (dd, J = 17.4 Hz, 7.9 Hz, 1H), 1.40 (d, J = 6.56 Hz, 3H) |
| 35 | 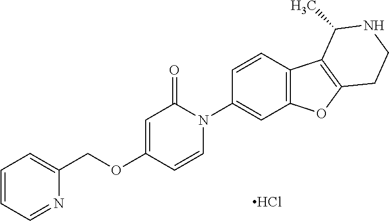 [SMILES: C[C@@H]1NCCc2oc3cc(-n4ccc(OCc5ccccn5)cc4=O)ccc3c21 ; Cl] | 57 | 388 | ¹H NMR (500 MHz, DMSO-d₆) δ 9.67 (br s, 1H), 9.26 (br s, 1H), 8.63 (m, 1H), 7.91 (t, J = 7.7 Hz, 1H), 7.69 (m, 2H), 7.62 (m, 2H), 7.42 (t, J = 4.9 Hz, 1H), 7.26 (m, 1H), 6.17 (d, J = 7.6 Hz, 1H), 5.98 (s, 1H), 5.23 (s, 2H), 4.79 (br s, 1H), 3.64 (m, 1H), 3.47 (m, 2H), 3.10 (m, 2H), 1.68 (d, J = 6.81 Hz, 3H) |
| 36 | 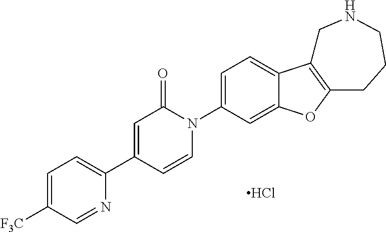 [SMILES: O=c1cc(-c2ccc(C(F)(F)F)cn2)ccn1-c1ccc2c3c(oc2c1)CCCNC3 ; Cl] | 39 | 426 | ¹H NMR (500 MHz, DMSO-d₆) δ 9.28 (s, 2H), 9.15-9.14 (m, 1H), 8.41-8.34 (m, 2H), 7.86 (d, J = 7.5 Hz, 1H), 7.82 (d, J = 8.0 Hz, 1H), 7.76 (d, J = 2.0 Hz, 1H), 7.39-7.37 (m, 1H), 7.29 (d, J = 2.0 Hz, 1H), 7.10-7.08 (m, 1H), 4.43 (s, 2H), 3.50-3.48 (m, 2H), 3.13 (t, J = 6.0 Hz, 2H), 2.12-2.08 (m, 2H) |
| 37 | 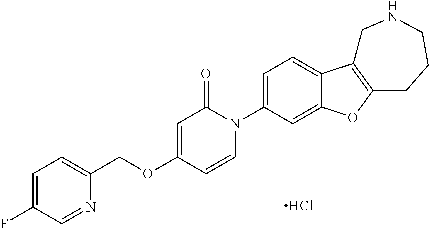 [SMILES: O=c1cc(OCc2ccc(F)cn2)ccn1-c1ccc2c3c(oc2c1)CCCNC3 ; Cl] | 4.5 | 406 | ¹H NMR (500 MHz, DMSO-d₆) δ 9.34 (s, 2H), 8.62 (d, J = 2.5 Hz, 1H), 7.85-7.81 (m, 1H), 7.76 (d, J = 8.0 Hz, 1H), 7.67-7.64 (m, 1H), 7.62- 7.59 (m, 2H), 7.25 (d, J = 8.0 Hz, 1H), 6.16-6.14 (m, 1H), 5.98 (d, J = 8.0 Hz, 1H), 5.22 (s, 2H), 4.41 (s, 2H), 3.47 (s, 2H), 3.11 (t, J = 6.0 Hz, 2H), 2.10-2.07 (m, 2H) |
| 38 | 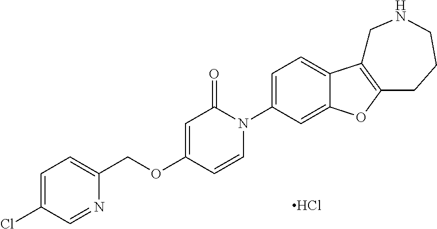 [SMILES: O=c1cc(OCc2ccc(Cl)cn2)ccn1-c1ccc2c3c(oc2c1)CCCNC3 ; Cl] | 3 | 422 | ¹H NMR (500 MHz, DMSO-d₆) δ 9.23 (s, 2H), 8.68-8.67 (m, 1H), 8.04-8.02 (m, 1H), 7.75 (d, J = 8.5 Hz, 1H), 7.63-7.60 (m, 3H), 7.26-7.24 (m, 1H), 6.18-6.16 (m, 1H), 5.97 (d, J = 3.0 Hz, 1H), 5.24 (s, 2H), 4.41 (s, 2H), 3.49-3.47 (m, 2H), 3.11 (t, J = 6.0 Hz, 2H). 2.11-2.07 (m, 2H) |
| 39 | 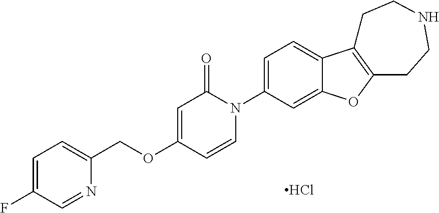 [SMILES: O=c1cc(OCc2ccc(F)cn2)ccn1-c1ccc2c3c(oc2c1)CCNCC3 ; Cl] | 9 | 406 | ¹H NMR (500 MHz, DMSO-d₆) δ 9.43 (s, 2H), 8.62 (d, J = 3.0 Hz, 1H), 7.85-7.81 (m, 1H), 7.67-7.63 (m, 2H), 7.60-7.58 (m, 2H), 7.23-7.21 (m, 1H), 6.16-6.14 (m, 1H), 5.98 (d, J = 2.5 Hz, 1H), 5.22 (s, 2H), 3.43-3.37 (m, 4H), 3.30-3.28 (m, 2H), 3.08 (t, J = 5.5 Hz, 2H) |
| 40 | 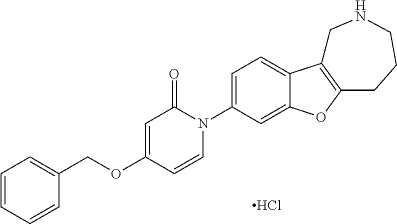 [SMILES: O=c1cc(OCc2ccccc2)ccn1-c1ccc2c3c(oc2c1)CCCNC3 ; Cl] | 3.4 | 387 | ¹H NMR (500 MHz, DMSO-d₆) δ 9.35 (s, 2H), 7.75 (d, J = 8.5 Hz, 1H), 7.61 (d, J = 2.0 Hz, 1H), 7.59 (d, J = 7.5 Hz, 1H), 7.48-7.41 (m, 4H), 7.39- 7.36 (m, 1H), 7.26 (dd, J = 8.5, 2.0 Hz, 1H), 6.12 (dd. J = 7.5, 2.5 Hz, 1H), 5.98 (d, J = 2.5 Hz, 1H), 5.15 (s, 2H), 4.40 (s, 2H), 3.48-3.46 (m, 2H), 3.11 (t, J = 5.5 Hz, 2H), 2.11-2.07 (m, 2H) |
| 41 | 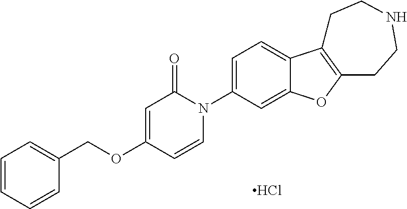 [SMILES: O=c1cc(OCc2ccccc2)ccn1-c1ccc2c3c(oc2c1)CCNCC3 ; Cl] | 5.7 | 387 | ¹H NMR (500 MHz, DMSO-d₆) δ 9.42 (s, 2H), 7.63 (d, J = 8.0 Hz, 1H), 7.58-7.57 (m, 2H), 7.48-7.37 (m, 4H), 7.39-7.37(m, 1H), 7.22 (dd, J = 8.5, 2.0 Hz, 1H), 6.11 (dd, J = 7.5, 2.5 Hz, 1H), 5.98 (d, J = 2.5 Hz, 1H), 5.15 (s, 2H), 3.43-3.37 (m, 4H), 3.30-3.37 (m, 2H), 3.07 (t, J = 5.5 Hz, 2H) |
| 42 | 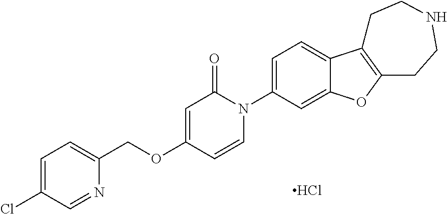 [SMILES: O=c1cc(OCc2ccc(Cl)cn2)ccn1-c1ccc2c3c(oc2c1)CCNCC3 ; Cl] | 4.7 | 422 | ¹H NMR (500 MHz, DMSO-d₆) δ 9.43 (s, 2H), 8.67(d, J = 2.5 Hz, 1H), 8.03 (dd, J = 8.5, 2.5 Hz, 1H), 7.64-7.58 (m, 4H), 7.22 (dd, J = 8.0, 2.0 Hz, 1H), 6.16 (dd, J = 8.0, 2.0 Hz, 1H), 5.96 (d, J = 3.0 Hz, 1H), 5.23 (s, 2H), 3.43-3.37 (m, 4H), 3.30-3.28 (m, 2H), 3.09-3.06 (m, 2H) |
| 43 | 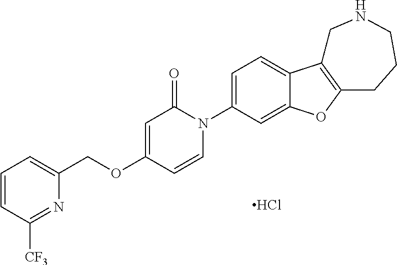 [SMILES: O=c1cc(OCc2cccc(C(F)(F)F)n2)ccn1-c1ccc2c3c(oc2c1)CCCNC3 ; Cl] | 9.7 | 456 | ¹H NMR (500 MHz, DMSO-d₆) δ 9.37 (s, 2H), 8.21 (t, J = 8.0 Hz, 1H), 7.94- 7.88 (m, 2H), 7.76 (d, J = 8.0 Hz, 1H), 7.64-7.62 (m, 2H), 7.25 (dd, J = 8.0, 2.0 Hz, 1H), 6.20 (dd, J = 7.5, 2.5 Hz, 1H), 6.00 (d, J = 2.5 Hz, 1H), 5.33 (s, 2H), 4.41 (s, 2H), 3.49-3.45 (m, 2H), 3.11 (t, J = 5.5 Hz, 2H), 2.12-1.96 (m, 2H) |
| 44 | 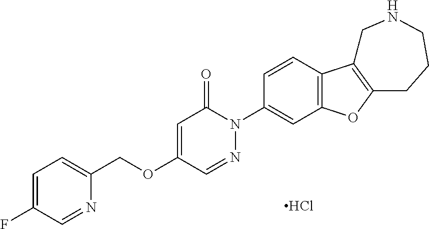 [SMILES: O=c1cc(OCc2ccc(F)cn2)cnn1-c1ccc2c3c(oc2c1)CCCNC3 ; Cl] | 13 | 407 | ¹H NMR (500 MHz, DMSO-d₆) δ 9.37 (s, 2H), 8.63 (d, J = 3.0 Hz, 1H), 8.02 (d, J = 3.0 Hz, 1H), 7.88-7.82 (m, 1H), 7.76 (d, J = 8.5 Hz, 1H), 7.72- 7.68 (m, 2H), 7.40 (dd, J = 8.5, 2.0 Hz, 1H), 6.65 (d, J = 2.5 Hz, 1H), 5.30 (s, 2H), 4.40 (s, 2H), 3.48-3.45 (m, 2H), 3.11 (t, J = 6.0 Hz, 2H), 2.15- 2.05 (m, 2H) |
| 45 | 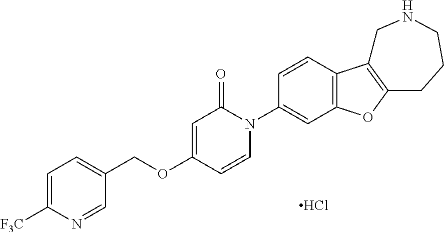 [SMILES: O=c1cc(OCc2ccc(C(F)(F)F)nc2)ccn1-c1ccc2c3c(oc2c1)CCCNC3 ; Cl] | 29 | 456 | ¹H NMR (500 MHz, DMSO-d₆) δ 9.42 (s, 2H), 8.89 (s, 1H), 8.20-8.18 (m, 1H), 7.99 (d, J = 8.5 Hz, 1H), 7.76 (d, J = 8.0 Hz, 1H), 7.64-7.62 (m, 2H), 7.26-7.24 (m, 1H), 6.18-6.16 (m, 1H), 6.03 (d, J = 2.5 Hz, 1H), 5.35 (s, 2H), 4.40 (s, 2H), 3.48-3.46 (m, 2H), 3.11 (t, J = 5.5 Hz, 2H), 2.10-2.08 (m, 2H) |
| 46 | 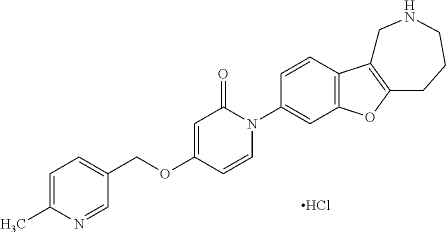 [SMILES: Cc1ccc(COc2ccn(-c3ccc4c5c(oc4c3)CCCNC5)c(=O)c2)cn1 ; Cl] | 47 | 402 | ¹H NMR (500 MHz, DMSO-d₆) δ 9.33 (s, 2H), 8.63 (s, 1H), 7.94 (s, 1H), 7.76 (d, J = 8.5 Hz, 1H), 7.62-7.59 (m, 2H), 7.46 (s, 1H), 7.25 (dd, J = 8.5, 1.5 Hz, 1H), 6.13-6.11 (m, 1H), 6.02 (d, J = 3.0 Hz, 1H), 5.20 (S, 2H), 4.41 (s, 2H), 3.47 (s, 2H), 3.11 (t, J = 5.5 Hz, 2H), 2.55 (s, 3H), 2.09 (s, 2H) |
| 47 | 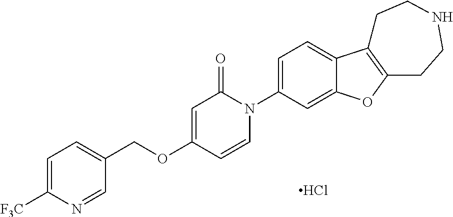 [SMILES: O=c1cc(OCc2ccc(C(F)(F)F)nc2)ccn1-c1ccc2c3c(oc2c1)CCNCC3 ; Cl] | 55 | 456 | ¹H NMR (500 MHz, DMSO-d₆) δ 9.41 (s, 2H), 8.89 (s, 1H), 8.20-8.18 (m, 1H), 7.99 (d, J = 8.0 Hz, 1H), 7.65- 7.59 (m, 3H), 7.24-7.22 (m, 1H), 6.17-6.15 (m, 1H), 6.03 (d, J = 2.5 Hz, 1H), 5.35 (s, 2H), 3.40 (t, J = 5.5 Hz, 4H), 3.30-3.28 (m, 2H), 3.08 (t, J = 5.5 Hz, 2H) |
| 48 | 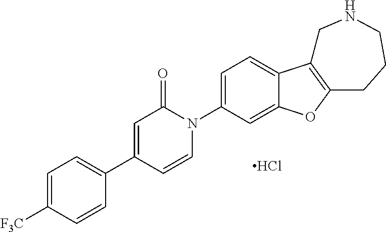 [SMILES: O=c1cc(-c2ccc(C(F)(F)F)cc2)ccn1-c1ccc2c3c(oc2c1)CCCNC3 ; Cl] | 4.5 | 425 | ¹H NMR (500 MHz, DMSO-d₆) δ 9.36 (s, 2H), 8.02-8.00 (m, 2H), 7.89-7.87 (m, 2H), 7.82 (t, J = 8.0 Hz, 2H), 7.74 (d, J = 1.5 Hz, 1H), 7.37-7.35 (m, 1H), 6.88 (d, J = 2.0 Hz, 1H), 6.76- 6.74 (m, 1H), 4.42 (s, 2H), 3.49-3.47 (m, 2H), 3.13 (t, J = 6.0 Hz, 2H), 2.10 (s, 2H) |
| 49 | 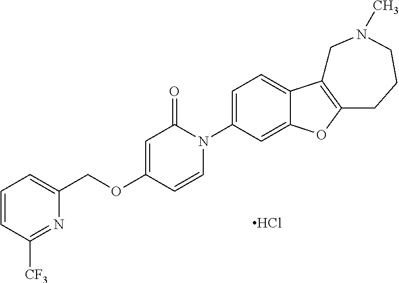 [SMILES: CN1CCCc2oc3cc(-n4ccc(OCc5cccc(C(F)(F)F)n5)cc4=O)ccc3c2C1 ; Cl] | 22 | 470 | ¹H NMR (500 MHz, DMSO-d₆) δ 10.46 (s, 1H), 8.21 (t. J = 8.0 Hz, 1H), 7.93 (d, J = 8.0 Hz, 1H), 7.89 (d, J = 8.0 Hz, 1H), 7.73 (d, J = 8.0 Hz, 1H), 7.65-7.62 (m, 2H), 7.28 (dd, J = 8.0, 2.0 Hz, 1H), 6.22-6.18 (m, 1H), 6.00 (d, J = 3.0 Hz, 1H), 5.33 (s, 2H), 4.69-4.48 (m, 2H), 3.68-3.50 (m, 2H), 3.10 (t, J = 5.5 Hz, 2H), 2.90 (s, 3H), 2.27-2.05 (m, 2H) |
| 50 | 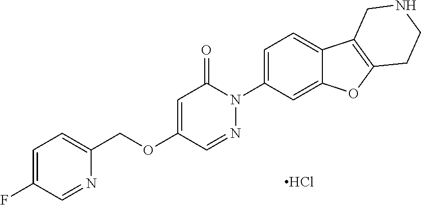 [SMILES: O=c1cc(OCc2ccc(F)cn2)cnn1-c1ccc2c3c(oc2c1)CCNC3 ; Cl] | 36 | 393 | ¹H NMR (500 MHz, DMSO-d₆) δ 9.49 (s, 2H), 8.64 (d, J = 3.0 Hz, 1H), 8.03 (d, J = 2.5 Hz, 1H), 7.85 (td, J = 8.8, 3.0 Hz, 1H), 7.79 (d, J = 1.5 Hz, 1H), 7.72-7.68 (m, 2H), 7.43 (dd, J = 8.3, 1.8 Hz, 1H), 6.56 (d, J = 3.0 Hz, 1H), 5.30 (s, 2H), 4.36 (s, 2H), 3.56 (s, 2H), 3.12 (t, J = 5.8 Hz, 2H) |
| 51 | 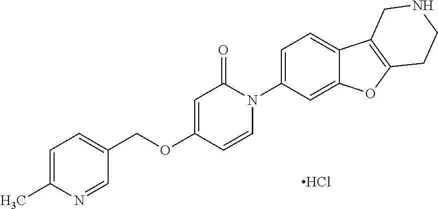 [SMILES: Cc1ccc(COc2ccn(-c3ccc4c5c(oc4c3)CCNC5)c(=O)c2)cn1 ; Cl] | 145 | 388 | ¹H NMR (500 MHz, DMSO-d₆) δ 9.68 (s, 2H), 8.69 (s, 1H), 8.06 (d, J = 8.0 Hz, 1H), 7.70-7.67 (m, 2H), 7.62 (d, J = 8.0 Hz, 1H), 7.56 (d, J = 8.0 Hz, 1H), 7.27 (dd, J = 8.3, 1.8 Hz, 1H), 6.13 (dd, J = 7.5, 3.0 Hz, 1H), 6.03 (d, J = 2.5 Hz, 1H), 5.23 (s, 2H), 4.35 (s, 2H), 3.56-3.53 (m, 2H), 3.12 (t, J = 5.5 Hz, 2H), 2.60 (s, 3H) |
| 52 | 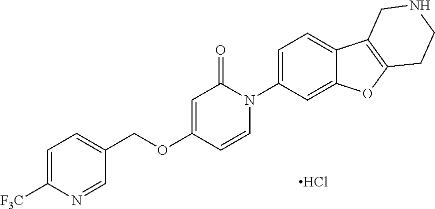 [SMILES: O=c1cc(OCc2ccc(C(F)(F)F)nc2)ccn1-c1ccc2c3c(oc2c1)CCNC3 ; Cl] | 73 | 442 | ¹H NMR (500 MHz, DMSO-d₆) δ 9.43 (s, 2H), 8.89 (d, J = 1.0 Hz, 1H), 8.19 (dd, J = 7.8, 1.3 Hz, 1H), 8.00 (d, J = 8.0 Hz, 1H), 7.70-7.69 (m, 2H), 7.64 (d, J = 7.5 Hz, 1H), 7.28 (dd, J = 8.5, 2.0 Hz, 1H), 6.17 (dd, J = 7.5, 3.0 Hz, 1H), 6.04 (d, J = 2.5 Hz, 1H), 5.35 (s, 2H), 4.36 (s, 2H), 3.56 (s, 2H), 3.12 (t, J = 5.8 Hz, 2H) |
| 53 | 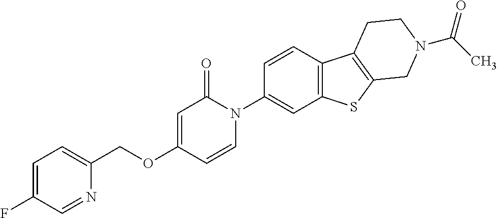 [SMILES: CC(=O)N1CCc2c(sc3cc(-n4ccc(OCc5ccc(F)cn5)cc4=O)ccc23)C1] | 39 | 450 | ¹H NMR (500 MHz, CDCl₃) δ 8.14 (d, J = 2.4 Hz, 1H), 7.79-7.66 (dd, J = 4.8, 1.6 Hz, 1H), 7.70-7.64 (m, 1H), 7.50-7.44 (m, 2H), 7.37-7.35 (m, 1H), 7.30 (d, J = 7.6 Hz, 1H), 6.12- 6.10 (dd, J = 7.6, 2.5 Hz, 1H), 6.06 (d, J = 2.5 Hz, 1H), 5.17 (s, 2H), 4.90- 4.76 (m, 2H), 4.00-3.82 (m, 2H), 2.94-2.87 (m, 2H), 2.24-2.21 (m, 3H) |
| 54 | 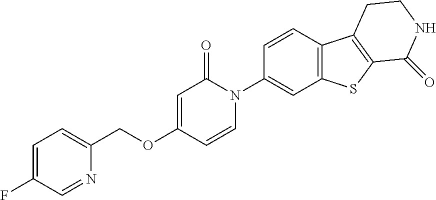 [SMILES: O=C1NCCc2c1sc1cc(-n3ccc(OCc4ccc(F)cn4)cc3=O)ccc21] | 60% inhibition @ 1 uM | 422 | ¹H NMR (500 MHz, DMSO-d₆) δ 8.62 (d, J = 2.9 Hz, 1H), 8.12 (d, J = 1.8 Hz, 1H), 8.02-7.97 (m, 2H), 7.84- 7.80 (td, J = 8.7, 2.9 Hz, 1H), 7.68 7.65 (m, 2H), 7.47-7.45 (dd, J = 8.5, 1.9 Hz, 1H), 6.18-6.16 (dd, J = 7.6, 2.7 Hz, 1H), 6.01 (d, J = 2.7 Hz, 1H), 5.22 (s, 2H), 3.60-3.57 (td, J = 7.2, 2.5 Hz, 2H), 3.08 (t, J = 7.1 Hz, 2H) |
| 55 | 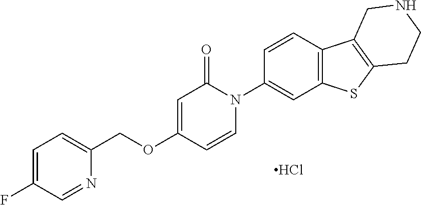 [SMILES: O=c1cc(OCc2ccc(F)cn2)ccn1-c1ccc2c3c(sc2c1)CCNC3 ; Cl] | 2.7 | 408 | ¹H NMR (500 MHz, DMSO-d₆) δ 9.70 (s, 2H), 8.62 (d, J = 2.9 Hz, 1H), 8.04 (d, J = 1.9 Hz, 1H), 7.85-7.81 (m, 2H), 7.67-7.62 (m, 2H), 7.40- 7.38 (dd, J = 8.5, 1.9 Hz, 1H), 6.17- 6.16 (dd, J = 7.6, 2.7 Hz, 1H), 6.00 (d, J = 2.7 Hz, 1H), 5.22 (s, 2H), 4.42 (s, 2H), 3.52-3.47 (m, 2H), 3.21-3.18 (m, 2H) |
| 56 | 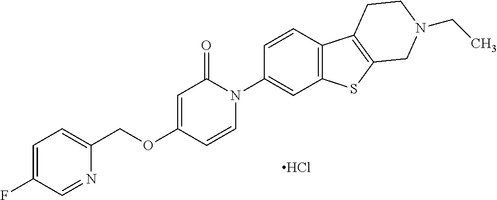 [SMILES: CCN1CCc2c(sc3cc(-n4ccc(OCc5ccc(F)cn5)cc4=O)ccc23)C1 ; Cl] | 9.5 | 436 | ¹H NMR (500 MHz, DMSO-d₆) δ 10.9 (s, 1H), 8.62 (d, J = 2.9 Hz, 1H), 8.07 (d, J = 1.8 Hz, 1H), 7.85-7.79 (m, 2H), 7.68-7.62 (m, 2H), 7.43- 7.41 (dd, J = 8.4, 1.9 Hz, 1H), 6.18- 6.16 (dd, J = 7.6, 2.7 Hz, 1H), 6.01 (d, J = 2.7 Hz, 1H), 5.22 (s, 2H), 4.77 (d, J = 16.1 Hz, 1H), 4.50-4.45 (d, J = 16.1, 7.2 Hz, 1H), 3.87-3.82 (m, 1H), 3.40-3.26 (m, 3H), 3.23-3.13 (m, 2H), 1.37 (t, J = 7.2 Hz, 3H) |
| 57 | 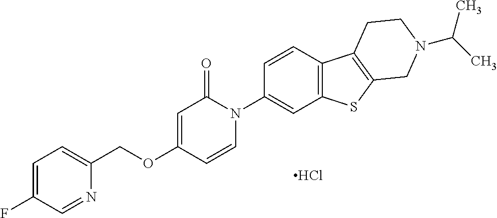 [SMILES: CC(C)N1CCc2c(sc3cc(-n4ccc(OCc5ccc(F)cn5)cc4=O)ccc23)C1 ; Cl] | 11 | 450 | ¹H NMR (500 MHz, DMSO-d₆) δ 10.8 (s, 1H), 8.62 (d, J = 2.9 Hz, 1H), 8.06 (d, J = 1.9 Hz, 1H), 7.86-7.81 (m, 2H), 7.67-7.63 (m, 2H), 7.43- 7.41 (dd, J = 8.6, 1.9 Hz, 1H), 6.18- 6.16 (dd, J = 7.6, 2.7 Hz, 1H), 6.01 (d, J = 2.7 Hz, 1H), 5.22 (s, 2H), 4.66- 4.62 (dd, J = 16.1, 2.1 Hz, 1H), 4.59- 4.54 (d, J = 16.1, 8.6 Hz, 1H), 3.86- 3.79 (m, 1H), 3.74-3.65 (m, 1H), 3.43-3.39 (m, 1H), 3.23-3.18 (m, 2H), 1.39 (t, J = 7.2 Hz, 6H) |
| 58 | 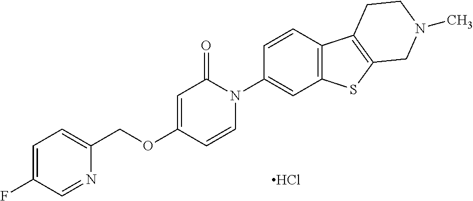 [SMILES: CN1CCc2c(sc3cc(-n4ccc(OCc5ccc(F)cn5)cc4=O)ccc23)C1 ; Cl] | 6.2 | 422 | ¹H NMR (500 MHz, DMSO-d₆) δ 11.1 (s, 1H), 8.62 (d, J = 2.9 Hz, 1H), 8.06 (d, J = 1.8 Hz, 1H), 7.85-7.80 (m, 2H), 7.68-7.63 (m, 2H), 7.42- 7.41 (dd, J = 8.5, 1.9 Hz, 1H), 6.17- 6.15 (dd, J = 7.6, 2.7 Hz, 1H), 6.01 (d, J = 2.8 Hz, 1H), 5.22 (s, 2H), 4.72 (d, J = 16.1 Hz, 1H), 4.50-4.46 (d, J = 16.1, 7.5 Hz, 1H), 3.82-3.75 (m, 1H), 3.53-3.43 (m, 1H), 3.19-3.16 (m, 2H), 2.97 (d, J = 3.8 Hz, 3H) |
| 59 | 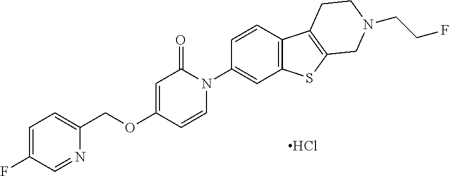 [SMILES: O=c1cc(OCc2ccc(F)cn2)ccn1-c1ccc2c3c(sc2c1)CN(CCF)CC3 ; Cl] | 36 | 454 | ¹H NMR (500 MHz, DMSO-d₆) δ 11.36 (s, 1H), 8.62 (d, J = 2.8 Hz, 1H), 8.07 (d, J = 1.7 Hz, 1H), 7.84- 7.80 (m, 2H), 7.67-7.63 (m, 2H), 7.43-7.41 (dd, J = 8.4, 1.8 Hz, 1H), 6.18-6.16 (dd, J = 7.6, 2.6 Hz, 1H), 6.01 (d, J = 2.7 Hz, 1H), 5.27 (s, 2H), 5.03-5.02 (m, 1H), 4.94-4.92 (m, 1H), 4.85-4.74 (m, 1H), 4.65-4.57 (m, 1H), 3.94-3.63 (m, 4H), 3.23- 3.18 (m, 2H) |
| 60 | 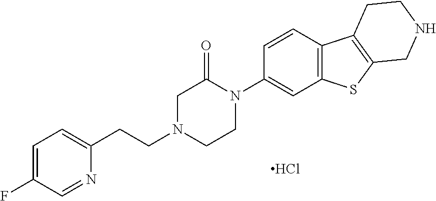 [SMILES: O=C1CN(CCc2ccc(F)cn2)CCN1c1ccc2c3c(sc2c1)CNCC3 ; Cl] | 118 | 411 | ¹H NMR (500 MHz, CD₃OD) δ 8.40 (d, J = 2.7 Hz, 1H), 7.88 (d, J = 1.4 Hz, 1H), 7.80 (dd, J = 8.4, Hz, 1H), 7.60-7.56 (td, J = 8.4, 2.9 Hz, 1H), 7.46-7.43 (dd, J = 8.4, 4.4 Hz, 1H), 7.40-7.43 (dd, J = 8.5, 1.7 Hz, 1H), 4.55 (s, 2H), 3.81-3.78 (m, 2H), 3.65 (t, J = 6.1 Hz, 2H), 3.61-3.53 (m, 2H), 3.19-3.09 (m, 8H) |
| 61 | 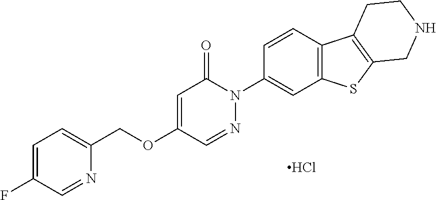 [SMILES: O=c1cc(OCc2ccc(F)cn2)cnn1-c1ccc2c3c(sc2c1)CNCC3 ; Cl] | 34 | 409 | ¹H NMR (500 MHz, DMSO-d₆) δ 9.45 (s, 2H), 8.64 (d, J = 2.8 Hz, 1H), 8.18 (d, J = 1.8 Hz, 1H), 8.04 (d, J = 2.8 Hz, 1H), 7.87-7.81 (m, 2H), 7.72- 7.69 (dd, J = 8.6, 4.5, 1H), 7.57-7.55 (dd, J = 8.5, 1.8 Hz, 1H), 6.58 (d, J = 2.8 Hz, 1H), 5.30 (s, 2H), 4.50 (s, 2H), 3.53 (t, J = 5.8 Hz, 2H), 3.09 (t, J = 5.8 Hz, 2H) |
| 62 | 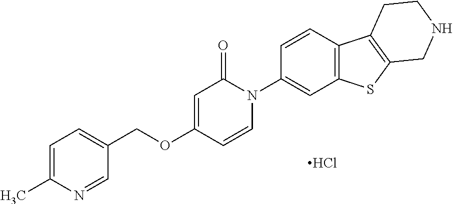 [SMILES: Cc1ccc(COc2ccn(-c3ccc4c5c(sc4c3)CNCC5)c(=O)c2)cn1 ; Cl] | 103 | 404 | ¹H NMR (500 MHz, DMSO-d₆) δ 9.80 (s, 2H), 8.70 (s, 1H), 8.10 (d, J = 7.6 Hz, 1H), 8.05 (d, J = 1.8 Hz, 1H), 7.83 (d, J = 8.4 Hz, 1H), 7.65 (d, J = 7.6, 1H), 7.60 (d, J = 8.0 Hz, 1H), 7.41-7.39 (dd, J = 8.4, 1.8 Hz, 1H), 6.15-6.13 (dd, J = 7.6, 2.7 Hz, 1H), 6.08 (d, J = 2.6 Hz, 1H), 5.24 (s, 2H), 4.48 (s, 2H), 3.53-3.50 (m, 2H), 3.10 3.06 (m, 2H), 2.64 (s, 3H) |
| 63 | 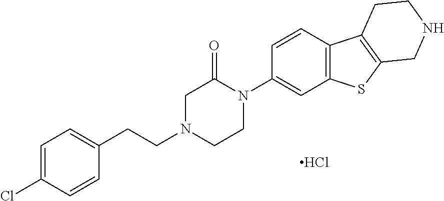 [SMILES: O=C1CN(CCc2ccc(Cl)cc2)CCN1c1ccc2c3c(sc2c1)CNCC3 ; Cl] | 33 | 426 | ¹H NMR (500 MHz, CD₃OD) δ 7.92 (d, J = 1.6 Hz, 1H), 7.81 (d, J = 8.6 Hz, 1H), 7.44-7.42 (dd, J = 8.5, 1.6 Hz, 1H), 7.37-7.31 (m, 4H), 4.56 (s, 2H), 4.20-3.37 (m, 9H), 3.21-3.01 (m, 5H) |
| 64 | 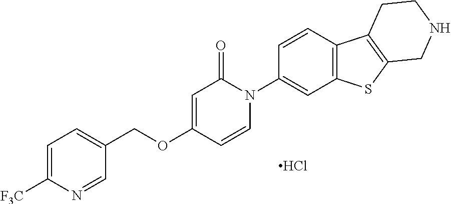 [SMILES: O=c1cc(OCc2ccc(C(F)(F)F)nc2)ccn1-c1ccc2c3c(sc2c1)CNCC3 ; Cl] | 48 | 458 | ¹H NMR (500 MHz, DMSO-d₆) δ 9.62 (s, 2H), 8.89 (s, 1H), 8.19 (d, J = 7.9 Hz, 1H), 8.06 (d, J = 1.8 Hz, 1H), 7.99 (d, J = 8.0 Hz, 1H), 7.83 (d, J = 8.4, 1H), 7.66 (d, J = 7.6 Hz, 1H), 7.41-7.40 (dd, J = 8.4, 1.8 Hz, 1H), 6.19-6.17 (dd, J = 7.6, 2.7 Hz, 1H), 6.05 (d, J = 2.7 Hz, 1H), 5.35 (s, 2H), 4.49 (s, 2H), 3.53-3.50 (m, 2H), 3.09- 3.07 (m, 2H) |
| 65 | 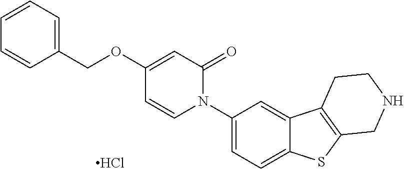 [SMILES: O=c1cc(OCc2ccccc2)ccn1-c1ccc2sc3c(c2c1)CCNC3 ; Cl] | 360 | 389 | ¹H NMR (300 MHz, DMSO-d₆) δ 9.51 (s, 2H), 8.09 (d, J = 8.7 Hz, 1H), 7.75 (d, J = 1.8 Hz, 1H), 7.64 (d, J = 7.8 Hz, 1H), 7.49-7.36 (m, 6H), 6.16- 6.13 (m, 1H), 6.00 (d, J = 2.7 Hz, 1H), 5.16 (s, 2H), 4.50 (s, 2H), 3.51 (s, 2H), 3.04 (br s, 2H) |
| 66 | 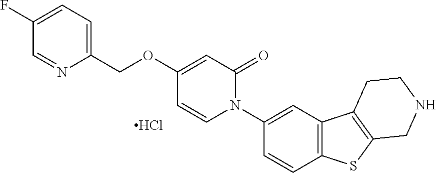 [SMILES: O=c1cc(OCc2ccc(F)cn2)ccn1-c1ccc2sc3c(c2c1)CCNC3 ; Cl] | 34% inhibition @ 1 um | 408 | ¹H NMR (400 MHz, DMSO-d₆) δ 9.69 (s, 2H), 8.62 (d, J = 2.8 Hz, 1H), 8.08 (d, J = 8.4 Hz, 1H), 7.86-7.81 (m, 1H), 7.75 (d, J = 2.0 Hz, 1H), 7.67-7.64 (m, 2H), 7.38-7.36 (m, 1H), 6.19-6.16 (m, 1H), 6.00 (d, J = 2.8 Hz, 1H), 5.23 (s, 2H), 4.46 (s, 2H), 3.50-3.48 (br s, 2H), 3.05 (s, 2H) |
| 67 | 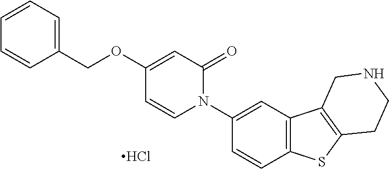 [SMILES: O=c1cc(OCc2ccccc2)ccn1-c1ccc2sc3c(c2c1)CNCC3 ; Cl] | 31% inhibition @ 1 um | 389 | ¹H NMR (300 MHz, DMSO-d₆) δ 9.73 (s, 2H), 8.06 (d, J = 8.4 Hz, 1H), 7.77 (d, J = 2.1 Hz, 1H), 7.63 (d, J = 7.5 Hz, 1H), 7.49-7.35 (m, 6H), 6.16- 6.13 (m, 1H), 6.00 (d, J = 2.7 Hz, 1H), 5.17 (s, 2H), 4.37 (s, 2H), 3.50 (br s, 2H), 3.19 (t, J = 4.8 Hz, 2H) |
| 68 | 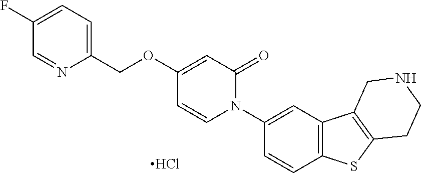 [SMILES: O=c1cc(OCc2ccc(F)cn2)ccn1-c1ccc2sc3c(c2c1)CNCC3 ; Cl] | 13% inhibition @ 1 um | 408 | ¹H NMR (300 MHz, DMSO-d₆) δ 9.88 (br s, 2H), 8.62 (d, J = 2.7 Hz, 1H), 8.06 (d, J = 8.4 Hz, 1H), 7.87- 7.78 (m, 2H), 7.68-7.64 (m, 2H), 7.38-7.35 (m, 1H), 6.20-6.16 (m, 1H), 6.01 (d, J = 2.7 Hz, 1H), 5.23 (s, 2H), 4.37 (s, 2H), 3.50 (br s, 2H), 3.20 (t, J = 5.1 Hz, 2H) |
| 69 | 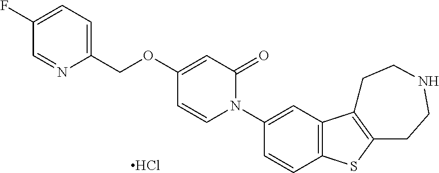 [SMILES: O=c1cc(OCc2ccc(F)cn2)ccn1-c1ccc2sc3c(c2c1)CCNCC3 ; Cl] | 69% inhibition @ 1 um | 422 | ¹H NMR (300 MHz, DMSO-d₆) δ 9.48 (br s, 2H), 8.62 (d, J = 3.0 Hz, 1H), 8.00 (d, J = 5.7 Hz, 1H), 7.86- 7.79 (m, 1H), 7.74 (d, J = 1.8 Hz, 1H), 7.68-7.61 (m, 2H), 7.30 (dd, J = 8.4, 2.0 Hz, 1H), 6.16 (dd, J = 6.9, 2.7 Hz, 1H), 6.00 (d, J = 2.7 Hz, 1H), 5.23 (s, 2H), 3.33 (br s, 6H), 3.28 (br s, 2H) |
| 70 | 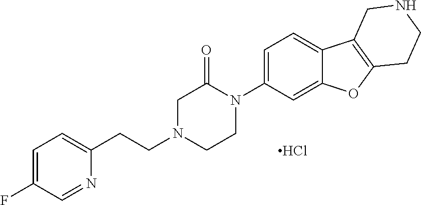 [SMILES: O=C1CN(CCc2ccc(F)cn2)CCN1c1ccc2c3c(oc2c1)CCNC3 ; Cl] | 66% inhibition @ 1 um | 395 | ¹H NMR (500 MHz, CD₃OD) δ 8.64 (d, J = 2.0 Hz, 1H), 7.87 (td, J = 8.5, 3.0 Hz, 1H), 7.69 (dd, J = 8.5, 4.5 Hz, 1H), 7.63-7.62 (m, 2H), 7.34 (dd, J = 8.3, 1.8 Hz, 1H), 4.45 (s, 2H), 4.27 (s, 2H), 4.13 (t, J = 5.5 Hz, 2H), 3.95 (t, J = 5.5 Hz, 2H), 3.84 (t, J = 7.3 Hz, 2H), 3.69 (t, J = 6.3 Hz, 2H), 3.51 (t, J = 7.3 Hz, 2H), 3.20 (t, J = 6.0 Hz, 2H) |
| 71 | 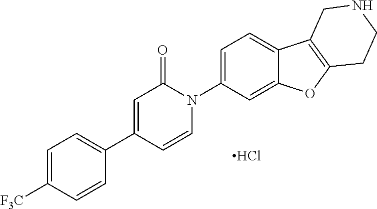 [SMILES: O=c1cc(-c2ccc(C(F)(F)F)cc2)ccn1-c1ccc2c3c(oc2c1)CCNC3 ; Cl] | 7.5 | 411 | ¹H NMR (500 MHz, DMSO-d₆) δ 9.46 (s, 2H), 8.02 (d, J = 8.0 Hz, 2H), 7.88 (d, J = 8.0 Hz, 2H), 7.84 (d, J = 7.0 Hz, 1H), 7.81 (d, J = 1.5 Hz, 1H), 7.74 (d, J = 8.0 Hz, 1H), 7.38 (dd, J = 8.3, 1.8 Hz, 1H), 6.90 (d, J = 1.5 Hz, 1H), 6.75 (dd, J = 7.5, 2.0 Hz, 1H), 4.38 (s, 2H), 3.57 (t, J = 6.0 Hz, 2H), 3.13 (t, J = 6.0 Hz, 2H) |
| 72 | 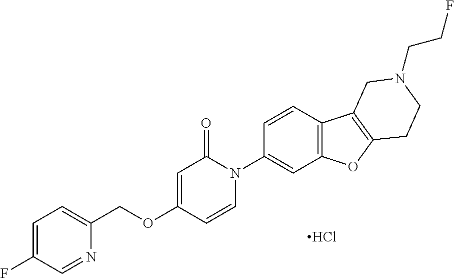 [SMILES: O=c1cc(OCc2ccc(F)cn2)ccn1-c1ccc2c3c(oc2c1)CCN(CCF)C3 ; Cl] | 29 | 438 | ¹H NMR (300 MHz, CD₃OD) δ 8.52 (d, J = 2.6, Hz, 1H), 7.79-7.56 (m, 5H), 7.30 (dd, J = 8.3, 1.8 Hz, 1H), 6.34 (dd, J = 7.7, 2.7 Hz, 1H), 6.13 (d, J = 2.1 Hz, 1H), 5.27 (s, 2H), 5.07 (t, J = 4.4 Hz, 1H), 4.96-4.86 (m, 1H), 4.86-4.77 (m, 1H), 4.60-4.50 (m, 1H), 4.04-3.94 (m, 1H), 3.89-3.84 (m, 1H), 3.81-3.75 (m, 2H), 3.41- 3.23 (m, 2H) |
| 73 | 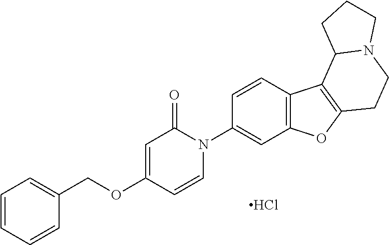 [SMILES: O=c1cc(OCc2ccccc2)ccn1-c1ccc2c3c(oc2c1)CCN1CCCC31 ; Cl] | 5.5 | 413 | ¹H NMR (300 MHz, CD₃OD) δ 7.70 (d, J = 8.3 Hz, 1H), 7.67-7.57 (m, 2H), 7.52-7.26 (m, 6H), 6.41-6.31 (m, 1H), 6.16 (d, J = 2.7 Hz, 1H), 5.20 (s, 2H), 5.13 (t, J = 6.9 Hz, 1H), 3.84- 3.67 (m, 3H), 3.56-3.40 (m, 1H), 3.30-3.14 (m, 2H), 2.87-2.61 (m, 1H), 2.40-2.11 (m, 3H) |
| 74 | 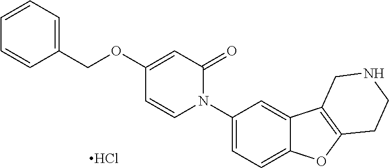 [SMILES: O=c1cc(OCc2ccccc2)ccn1-c1ccc2oc3c(c2c1)CNCC3 ; Cl] | 373 | 373 | ¹H NMR (300 MHz, CD₃OD) δ 7.77 (d, J = 7.5 Hz, 1H), 7.68-7.60 (m, 2H), 7.52-7.31 (m, 6H), 6.55 (d, J = 7.2 Hz, 1H), 6.31 (br s, 1H), 5.26 (s, 2H), 4.44 (s, 2H), 3.70 (t, J = 6.0 Hz, 2H), 3.22 (t, J = 5.7 Hz, 2H) |
| 75 | 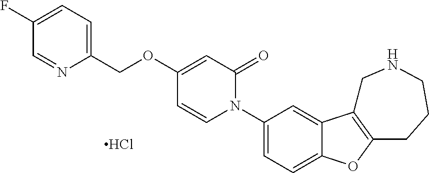 [SMILES: O=c1cc(OCc2ccc(F)cn2)ccn1-c1ccc2oc3c(c2c1)CNCCC3 ; Cl] | 34% inhibition @ 1 uM | 406 | ¹H NMR (300 MHz, DMSO-d₆) δ 9.64-9.51 (m, 2H), 8.62 (d, J = 2.7 Hz, 1H), 7.89-7.78 (m, 1H), 7.75 (d, J = 1.5 Hz, 1H), 7.71-7.54 (m, 3H), 7.25 (dd, J = 8.7, 1.5 Hz, 1H), 6.16 (dd, J = 7.5, 2.4 Hz, 1H), 5.99 (d, J = 2.4 Hz, 1H), 5.22 (s, 2H), 4.36 (br s, 2H), 3.52-3.38 (m, 2H), 3.18-3.03 (m, 2H), 2.18-2.02 (m, 2H) |
| 76 | 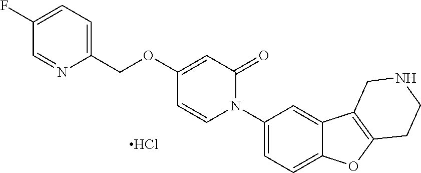 [SMILES: O=c1cc(OCc2ccc(F)cn2)ccn1-c1ccc2oc3c(c2c1)CNCC3 ; Cl] | 35% inhibition @ 1 uM | 392 | ¹H NMR (300 MHz, DMSO-d₆) δ 9.93-9.81 (m, 2H), 8.62 (d, J = 2.7 Hz, 1H), 7.89-7.78 (m, 1H), 7.73- 7.57 (m, 4H), 7.29 (dd, J = 8.7 Hz, 1.8 Hz, 1H), 6.16 (dd, J = 7.8 Hz, 2.4 Hz, 1H), 6.00 (d, J = 2.4 Hz, 1H), 5.22 (s, 2H), 4.28 (s, 2H), 3.59-3.47 (m, 2H), 3.18-3.08 (m, 2H) |
| 77 | 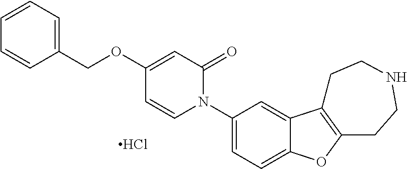 [SMILES: O=c1cc(OCc2ccccc2)ccn1-c1ccc2oc3c(c2c1)CCNCC3 ; Cl] | 44 | 387 | ¹H NMR (300 MHz, DMSO-d₆) δ 9.55-9.43 (m, 2H), 7.64 (m, 3H), 7.51-7.33 (m, 5H), 7.22 (dd, J = 8.4, 2.1 Hz, 1H), 6.11 (dd, J = 7.5, 2.7 Hz, 1H), 5.98 (d, J = 2.7 Hz, 1H), 5.16 (s, 2H), 3.47-3.23 (m, 6H), 3.12-3.00 (m, 2H) |
| 78 | 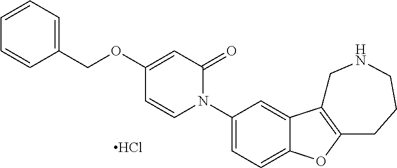 [SMILES: O=c1cc(OCc2ccccc2)ccn1-c1ccc2oc3c(c2c1)CNCCC3 ; Cl] | 35% inhibition @ 1 uM | 387 | ¹H NMR (400 MHz, DMSO-d₆) δ 9.46-9.36 (m, 2H), 7.75 (d, J = 2.0 Hz, 1H), 7.66-7.54 (m, 2H), 7.51- 7.34 (m, 5H), 7.25 (dd, J = 8.4, 2.0 Hz, 1H), 6.12 (dd, J = 7.6, 2.8 Hz, 1H), 5.99 (d, J = 2.8 Hz, 1H), 5.15 (s, 2H), 4.41-4.33 (m, 2H), 3.52-3.41 (m, 2H), 3.10 (t, J = 5.6 Hz, 2H), 2.15-2.02 (m, 2H) |
| 79 | 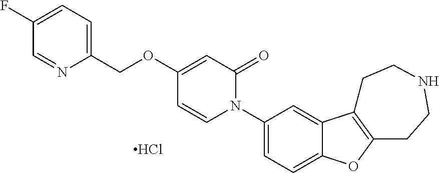 [SMILES: O=c1cc(OCc2ccc(F)cn2)ccn1-c1ccc2oc3c(c2c1)CCNCC3 ; Cl] | 78 | 406 | ¹H NMR (400 MHz, CD₃OD) δ 8.75 (s, 1H), 8.14-7.79 (m, 3H), 7.72-7.55 (m, 2H), 7.32 (d, J = 8.0 Hz, 1H), 6.73-6.60 (m, 1H), 6.42 (br s, 1H), 5.48 (s, 2H), 3.63-3.49 (m, 4H), 3.44- 3.35 (m, 2H), 3.22-3.10 (m, 2H) |
| 80 | 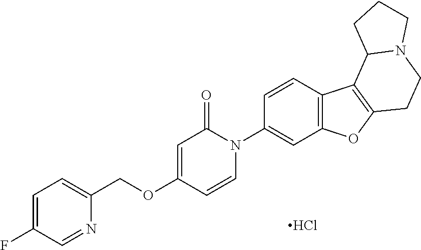 [SMILES: O=c1cc(OCc2ccc(F)cn2)ccn1-c1ccc2c3c(oc2c1)CCN1CCCC31 ; Cl] | 8.3 | 432 | ¹H NMR (300 MHz, CD₃OD) δ 8.62 (d, J = 2.7 Hz, 1H), 7.93-7.61 (m, 5H), 7.32 (dd, J = 8.3, 1.9 Hz, 1H), 6.43 (dd, J = 7.6, 2.7 Hz, 1H), 6.21 (d, J = 2.7 Hz, 1H), 5.34 (s, 2H), 5.15 (t, J = 6.9 Hz, 1H), 3.84-3.67 (m, 3H), 3.55-3.40 (m, 1H), 3.30-3.21 (m, 2H), 2.70 (dt, J = 14.4, 6.5 Hz, 1H), 2.38-2.14 (m, 3H) |
| 81 | 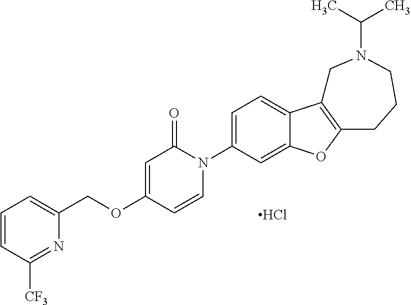 [SMILES: CC(C)N1CCCc2oc3cc(-n4ccc(OCc5cccc(C(F)(F)F)n5)cc4=O)ccc3c2C1 ; Cl] | 62 | 498 | ¹H NMR (500 MHz, DMSO-d₆) δ 9.97 (s, 1H), 8.21 (t, J = 8.0 Hz, 1H), 7.94- 7.83 (m, 3H), 7.66-7.63 (m, 2H), 7.30-7.28 (m, 1H), 6.21-6.19 (m, 1H), 6.00 (d, J = 3.0 Hz, 1H), 5.33 (s, 2H), 4.64-4.53 (m, 2H), 3.68-3.51 (m, 3H), 3.12 (s, 2H), 2.26-2.12 (m, 2H), 1.38-1.33 (m, 6H) |
| 82 | 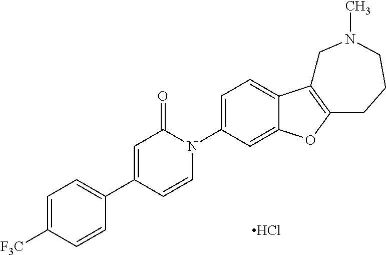 [SMILES: CN1CCCc2oc3cc(-n4ccc(-c5ccc(C(F)(F)F)cc5)cc4=O)ccc3c2C1 ; Cl] | 8.2 | 439 | ¹H NMR (500 MHz, DMSO-d₆) δ 10.68 (s, 1H), 8.02-8.00 (m, 2H), 7.89-7.87 (m, 2H), 7.84-7.82 (m, 1H), 7.80-7.76 (m, 2H), 7.39-7.37 (m, 1H), 6.89 (d, J = 2.0 Hz, 1H), 6.76-6.74 (m, 1H), 4.71-4.51 (m, 2H), 3.69-3.52 (m, 2H), 3.12 (t, J = 6.0 Hz, 2H), 2.91 (s, 3H), 2.19-2.12 (m, 2H) |
| 83 | 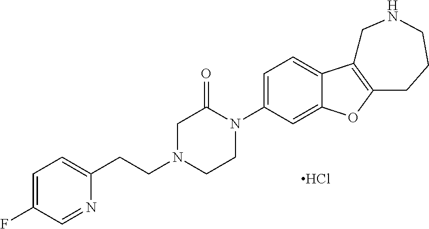 [SMILES: O=C1CN(CCc2ccc(F)cn2)CCN1c1ccc2c3c(oc2c1)CCCNC3 ; Cl] | 52 | 409 | ¹H NMR (500 MHz, DMSO-d₆) δ 9.21 (s, 2H), 8.49 (s, 1H), 7.67 (d, J = 8.0 Hz, 2H), 7.55 (d, J = 1.5 Hz, 1H), 7.45-7.42 (m, 1H), 7.25-7.23 (m, 1H), 4.37 (s, 2H), 3.68-3.46 (m, 4H), 3.27-2.65 (m, 10H), 2.09-2.05 (m, 2H) |
| 84 | 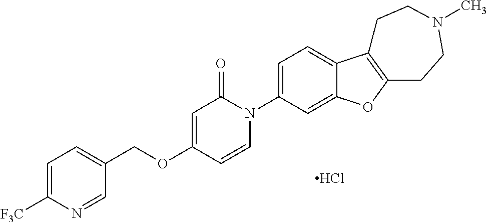 [SMILES: CN1CCc2oc3cc(-n4ccc(OCc5ccc(C(F)(F)F)nc5)cc4=O)ccc3c2CC1 ; Cl] | 29 | 470 | ¹H NMR (500 MHz, DMSO-d₆) δ 10.97 (s, 1H), 8.88 (s, 1H), 8.20-8.18 (m, 1H), 7.99 (d, J = 8.0 Hz, 1H), 7.66-7.59 (m, 3H), 7.24-7.22 (m, 1H), 6.17-6.15 (m, 1H), 6.03 (d, J = 3.0 Hz, 1H), 5.35 (s, 2H), 3.65-3.05 (m, 8H), 2.93 (s, 3H) |
| 85 | 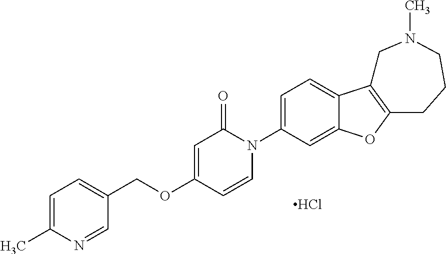 [SMILES: Cc1ccc(COc2ccn(-c3ccc4c5c(oc4c3)CCCN(C)C5)c(=O)c2)cn1 ; Cl] | 116 | 416 | ¹H NMR (500 MHz, DMSO-d₆) δ 10.60 (s, 1H), 8.59 (s, 1H), 7.87-7.86 (m, 1H), 7.73 (d, J = 8.5 Hz, 1H), 7.64-7.59 (m, 2H), 7.39 (d, J = 8.0 Hz, 1H), 7.28-7.26 (m, 1H), 6.13- 6.11 (m, 1H), 6.02 (d, J = 2.5 Hz, 1H), 5.18 (s, 2H), 4.72-4.70 (m, 1H), 4.48- 4.46 (m, 1H), 3.72-3.68 (m, 1H), 3.51-3.50 (m, 1H), 3.10 (t, J = 6.0 Hz, 2H), 2.90 (s, 3H), 2.52-2.50 (m, 3H), 2.19-2.09 (m, 2H) |
| 86 | 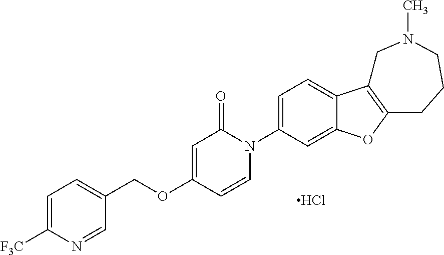 [SMILES: CN1CCCc2oc3cc(-n4ccc(OCc5ccc(C(F)(F)F)nc5)cc4=O)ccc3c2C1 ; Cl] | 53 | 470 | ¹H NMR (500 MHz, DMSO-d₆) δ 10.60 (s, 1H), 8.89 (s, 1H), 8.20-8.18 (m, 1H), 7.99 (d, J = 8.0 Hz, 1H), 7.74 (d, J = 8.0 Hz, 1H), 7.65-7.63 (m, 2H), 7.29-7.27 (m, 1H), 6.18-6.16 (m, 1H), 6.03 (d, J = 3.0 Hz, 1H), 5.35 (s, 2H), 4.72-4.46 (m, 2H), 3.69-3.50 (m, 2H), 3.10 (t, J = 6.0 Hz, 2H), 2.90 (s, 3H), 2.18-2.07 (m, 2H) |
| 87 | 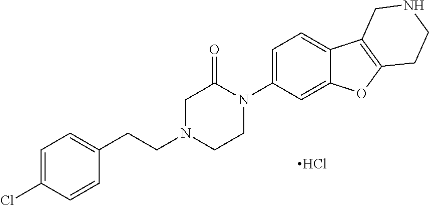 [SMILES: O=C1CN(CCc2ccc(Cl)cc2)CCN1c1ccc2c3c(oc2c1)CCNC3 ; Cl] | 58 | 410 | ¹H NMR (500 MHz, CD₃OD) δ 7.64 (d, J = 8.0 Hz, 1H), 7.60 (d, J = 1.5 Hz, 1H), 7.39-7.35 (m, 4H), 7.32 (dd, J = 8.3, 1.8 Hz, 1H), 4.45 (s, 2H), 4.21 (s, 2H), 4.10 (br s, 2H), 3.85 (br s, 2H), 3.69 (t, J = 6.3 Hz, 2H), 3.58 (t, J = 8.3 Hz, 2H), 3.21-3.16 (m, 4H) |
| 88 | 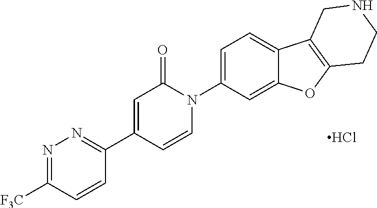 [SMILES: O=c1cc(-c2ccc(C(F)(F)F)nn2)ccn1-c1ccc2c3c(oc2c1)CCNC3 ; Cl] | 41% inhibition @ 1 uM | 413 | ¹H NMR (500 MHz, CD₃OD) δ 8.53 (d, J = 9.0 Hz, 1H), 8.27 (d, J = 9.0 Hz, 1H), 7.89 (d, J = 7.5 Hz, 1H), 7.74-7.72 (m, 2H), 7.44 (d, J = 1.5 Hz, 1H), 7.41 (dd, J = 8.3, 1.8 Hz, 1H), 7.34 (dd, J = 7.3, 1.8 Hz, 1H), 4.49 (s, 2H), 3.71 (t, J = 6.0 Hz, 2H), 3.23 (t, J = 6.0 Hz, 2H) |
| 89 | 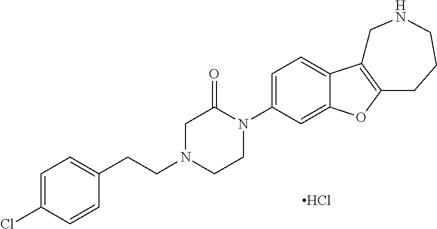 [SMILES: O=C1CN(CCc2ccc(Cl)cc2)CCN1c1ccc2c3c(oc2c1)CCCNC3 ; Cl] | 17 | 424 | ¹H NMR (500 MHz, DMSO-d₆) δ 9.33 (s, 2H), 7.66 (d, J = 8.5 Hz, 1H), 7.55 (d, J = 1.5 Hz, 1H), 7.36-7.30 (m, 4H), 7.24 (dd, J = 8.5, 1.5 Hz, 1H), 4.36 (s, 2H), 3.73-3.65 (m, 2H), 3.46- 3.44 (m, 2H), 3.25 (s, 2H), 3.08 (t, J = 6.0 Hz, 2H), 2.86-2.79 (m, 4H), 2.71- 2.66 (m, 2H), 2.17-2.12 (m, 2H) |
| 90 | 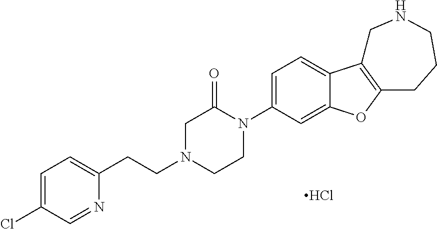 [SMILES: O=C1CN(CCc2ccc(Cl)cn2)CCN1c1ccc2c3c(oc2c1)CCCNC3 ; Cl] | 19 | 425 | ¹H NMR (500 MHz, DMSO-d₆) δ 9.29 (s, 2H), 8.55-8.54 (m, 1H), 7.87-7.85 (m, 1H), 7.66 (d, J = 8.0 Hz, 1H), 7.54 (d, J = 2.0 Hz, 1H), 7.42 (d, J = 8.0 Hz, 1H), 7.25-7.23 (m, 1H), 4.37 (s, 2H), 3.75-3.40 (m, 4H), 3.25-2.75 (m, 10H), 2.08-2.06 (m, 2H) |
| 91 | 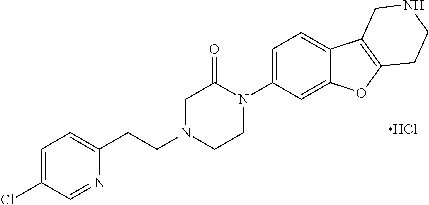 [SMILES: O=C1CN(CCc2ccc(Cl)cn2)CCN1c1ccc2c3c(oc2c1)CCNC3 ; Cl] | 84 | 411 | ¹H NMR (500 MHz, CD₃OD) δ 8.55 (d, J = 2.0 Hz, 1H), 7.84 (dd, J = 8.0, 2.5 Hz, 1H), 7.62 (d, J = 8.5 Hz, 1H), 7.57 (d, J = 1.0 Hz, 1H), 7.43 (d, J = 8.5 Hz, 1H), 7.29 (dd, J = 8.0, 1.5 Hz, 1H), 4.45 (s, 2H), 3.96 (s, 2H), 3.90 (br s, 2H), 3.69 (t, J = 6.3 Hz, 2H), 3.54 (br s, 2H), 3.47 (br s, 2H), 3.28- 3.26 (m, 2H), 3.19 (t, J = 6.0 Hz, 2H) |
| 92 | 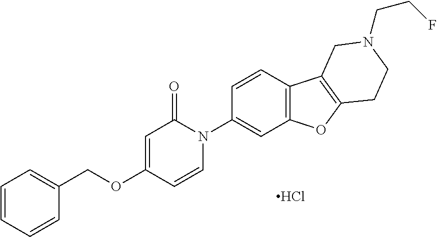 [SMILES: O=c1cc(OCc2ccccc2)ccn1-c1ccc2c3c(oc2c1)CCN(CCF)C3 ; Cl] | 25 | 419 | ¹H NMR (300 MHz, CD₃OD) δ 7.75- 7.62 (m, 3H), 7.52-7.27 (m, 6H), 6.41 (dd, J = 7.6, 2.7 Hz, 1H), 6.21 (d, J = 2.6 Hz, 1H), 5.22 (s, 2H), 5.07 (t, J = 4.4 Hz, 1H), 4.98-4.87 (m, 1H), 4.86-4.76 (m, 1H), 4.60-4.50 (m 1H), 4.10-3.95 (m, 1H), 3.90-3.84 (m, 1H), 3.81-3.75 (m, 2H), 3.41- 3.23 (m, 2H) |
| 93 | 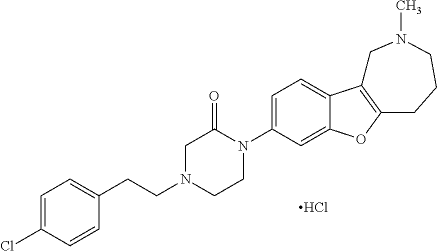 [SMILES: CN1CCCc2oc3cc(N4CCN(CCc5ccc(Cl)cc5)CC4=O)ccc3c2C1 ; Cl] | 69 | 438 | ¹H NMR (500 MHz, DMSO-d₆) δ 10.54 (s, 1H), 7.65 (d, J = 8.0 Hz, 1H), 7.56 (d, J = 1.5 Hz, 1H), 7.38- 7.24 (m, 5H), 4.71-4.37 (m, 2H), 3.79-3.41 (m, 5H), 3.27-3.21 (m, 2H), 3.07 (t, J = 6.0 Hz, 2H), 2.92- 2.77 (m, 8H), 2.23-2.132 (m, 2H) |
| 94 | 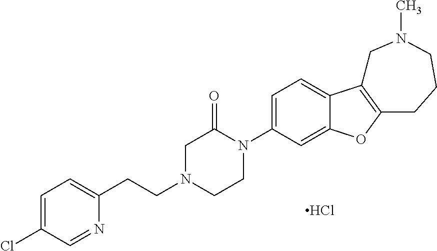 [SMILES: CN1CCCc2oc3cc(N4CCN(CCc5ccc(Cl)cn5)CC4=O)ccc3c2C1 ; Cl] | 103 | 439 | ¹H NMR (500 MHz, DMSO-d₆) δ 8.41 (s, 1H), 7.72 (dd, J = 8.5, 2.5 Hz, 1H), 7.55 (d, J = 8.0 Hz, 1H), 7.40 (d, J = 1.5 Hz, 1H), 7.32 (d, J = 8.5 Hz, 1H), 7.16 (dd, J = 8.0, 1.5 Hz, 1H), 4.59- 4.44 (m, 2H), 3.91-3.25 (m, 7H), 3.18-2.98 (m, 11H), 2.38-2.10 (m, 2H) |
| 95 | 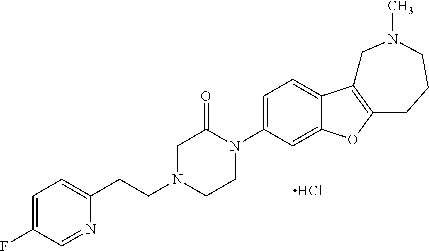 [SMILES: CN1CCCc2oc3cc(N4CCN(CCc5ccc(F)cn5)CC4=O)ccc3c2C1 ; Cl] | 62% inhibition @ 1 uM | 423 | ¹H NMR (500 MHz, DMSO-d₆) δ 8.34 (s, 1H), 7.56 (d, J = 8.5 Hz, 1H), 7.54- 7.49 (m, 1H), 7.42 (d, J = 1.5 Hz, 1H), 7.38-7.35 (m, 1H), 7.19 (dd, J = 8.5, 1.5 Hz, 1H), 4.66-4.39 (m, 2H), 3.95- 3.43 (m, 7H), 3.39-3.25 (m, 2H), 3.14-3.06 (m, 6H), 2.98 (s, 3H), 2.23- 2.09 (m, 2H) |
| 96 | 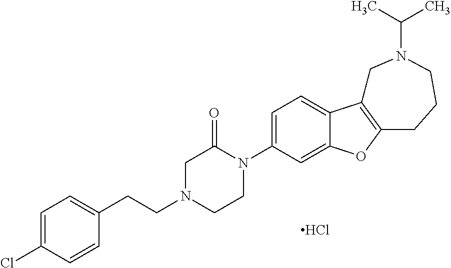 [SMILES: CC(C)N1CCCc2oc3cc(N4CCN(CCc5ccc(Cl)cc5)CC4=O)ccc3c2C1 ; Cl] | 150 | 466 | ¹H NMR (500 MHz, DMSO-d₆) δ 7.61 (d, J = 8.0 Hz, 1H), 7.43 (d, J = 1.5 Hz, 1H), 7.26-7.18 (m, 5H), 4.56- 4.46 (m, 2H), 3.90-3.40 (m, 9H), 3.20-2.85 (m, 7H), 2.32-2.05 (m, 2H), 1.40-1.35 (m, 6H) |
| 97 | 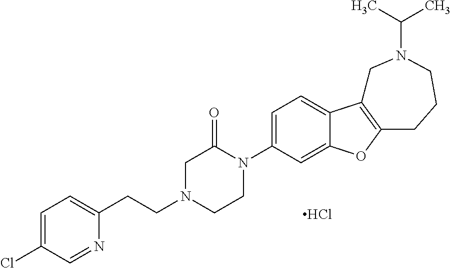 [SMILES: CC(C)N1CCCc2oc3cc(N4CCN(CCc5ccc(Cl)cn5)CC4=O)ccc3c2C1 ; Cl] | 66% inhibition @ 1 uM | 467 | ¹H NMR (500 MHz, DMSO-d₆) δ 8.41 (d, J = 1.5 Hz, 1H), 7.72 (dd, J = 8.5, 2.5 Hz, 1H), 7.59 (d, J = 8.0 Hz, 1H), 7.40 (d, J = 1.5 Hz, 1H), 7.31 (d, J = 8.5 Hz, 1H), 7.17 (dd, J = 8.0, 1.5 Hz, 1H), 4.55-4.45 (m, 2H), 3.80-3.40 (m, 8H), 3.20-2.90 (m, 8H), 2.35- 2.00 (m, 2H), 1.39-1.34 (m, 6H) |
| 98 | 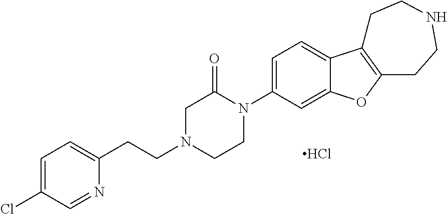 [SMILES: O=C1CN(CCc2ccc(Cl)cn2)CCN1c1ccc2c3c(oc2c1)CCNCC3 ; Cl] | 153 | 425 | ¹H NMR (500 MHz, CD₃OD) δ 8.39 (d, J = 2.5 Hz, 1H), 7.71 (dd, J = 8.5, 2.5 Hz, 1H), 7.47 (d, J = 8.0 Hz, 1H), 7.33-7.30 (m, 2H), 7.10 (dd, J = 8.0, 1.5 Hz, 1H), 3.69-3.67 (m, 2H), 3.45- 3.38 (m, 6H), 3.24-2.23 (m, 2H), 3.04-2.94 (m, 8H) |
| 99 | 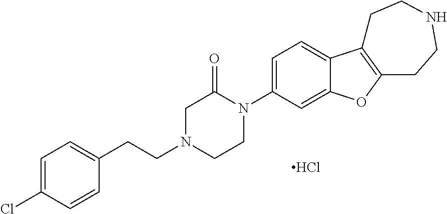 [SMILES: O=C1CN(CCc2ccc(Cl)cc2)CCN1c1ccc2c3c(oc2c1)CCNCC3 ; Cl] | 98 | 424 | ¹H NMR (500 MHz, CD₃OD) δ 7.47 (d, J = 8.5 Hz, 1H), 7.33 (d, J = 2.0 Hz, 1H), 7.22-7.17 (m, 4H), 7.11 (dd, J = 8.0, 1.5 Hz, 1H), 3.69-3.67 (m, 2H), 3.45-3.34 (m, 6H), 3.25-2.22 (m, 2H), 3.05-2.95 (m, 4H), 2.81- 2.75 (m, 4H) |
| 100 | 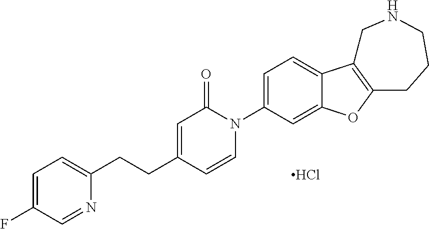 [SMILES: O=c1cc(CCc2ccc(F)cn2)ccn1-c1ccc2c3c(oc2c1)CCCNC3 ; Cl] | 8.1 | 404 | ¹H NMR (500 MHz, DMSO-d₆) δ 9.39 (s, 2H), 8.50 (d, J = 3.0 Hz, 1H), 7.77 (d, J = 8.5 Hz, 1H), 7.69-7.63 (m, 2H), 7.58 (d, J = 7.0 Hz, 1H), 7.43 (dd, J = 8.5, 4.5 Hz, 1H), 7.26 (dd, J = 8.0, 1.5 Hz, 1H), 6.31 (d, J = 1.0 Hz, 1H), 6.27 (dd, J = 7.0, 1.5 Hz, 1H), 4.40 (br s, 2H), 3.47 (br s, 2H), 3.12- 3.06 (m, 4H), 2.91-2.88 (m, 2H), 2.11-2.09 (m, 2H) |
| 101 | 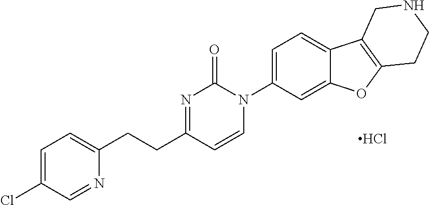 [SMILES: O=c1nc(CCc2ccc(Cl)cn2)ccn1-c1ccc2c3c(oc2c1)CCNC3 ; Cl] | 20 | 407 | ¹H NMR (500 MHz, DMSO-d₆) δ 9.79 (s, 2H), 8.57 (d, J = 2.5 Hz, 1H), 8.29 (d, J = 6.5 Hz, 1H), 7.90 (dd, J = 8.5, 2.5 Hz, 1H), 7.82 (d, J = 1.5 Hz, 1H), 7.74 (d, J = 8.0 Hz, 1H), 7.45 (d, J = 8.5 Hz, 1H), 7.38 (dd, J = 8.3, 1.8 Hz, 1H), 6.66 (d, J = 7.0 Hz, 1H), 4.35 (s, 2H), 3.56-3.53 (m, 2H), 3.23-3.21 (m, 2H), 3.15-3.12 (m, 4H) |
| 102 | 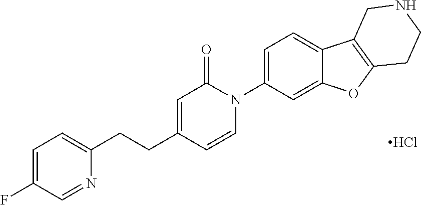 [SMILES: O=c1cc(CCc2ccc(F)cn2)ccn1-c1ccc2c3c(oc2c1)CCNC3 ; Cl] | 29 | 390 | ¹H NMR (500 MHz, DMSO-d₆) δ 9.64 (s, 2H), 8.54 (d, J = 2.5 Hz, 1H), 7.74-7.69 (m, 3H), 7.59 (d, J = 7.0 Hz, 1H), 7.46 (dd, J = 9.0, 4.5 Hz, 1H), 7.28 (dd, J = 8.3, 1.8 Hz, 1H), 6.32 (s, 1H), 6.28 (dd, J = 7.0, 2.0 Hz, 1H), 4.35 (s, 2H), 3.56-3.53 (m, 2H), 3.13-3.08 (m, 4H), 2.90 (t, J = 8.0 Hz, 2H) |
| 103 | 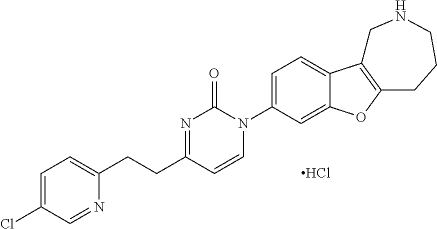 [SMILES: O=c1nc(CCc2ccc(Cl)cn2)ccn1-c1ccc2c3c(oc2c1)CCCNC3 ; Cl] | 8 | 421 | ¹H NMR (500 MHz, DMSO-d₆) δ 9.37 (s, 2H), 8.55 (d, J = 2.5 Hz, 1H), 8.10 (d, J = 6.5 Hz, 1H), 7.86 (dd, J = 8.0, 2.5 Hz, 1H), 7.79 (d, J = 8.5 Hz, 1H), 7.73 (d, J = 2.0 Hz, 1H), 7.42 (d, J = 8.0 Hz, 1H), 7.34 (dd, J = 8.5, 2.0 Hz, 1H), 6.53 (d, J = 8.0 Hz, 1H), 4.41 (s, 2H), 3.47 (s, 2H), 3.20-3.06 (m, 6H), 2.09 (s, 2H) |
| 104 | 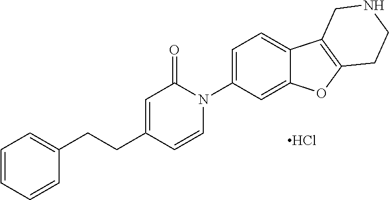 [SMILES: O=c1cc(CCc2ccccc2)ccn1-c1ccc2c3c(oc2c1)CCNC3 ; Cl] | 6.6 | 371 | ¹H NMR (500 MHz, DMSO-d₆) δ 9.52 (s, 2H), 7.71 (s, 1H), 7.70 (d, J = 7.0 Hz, 1H), 7.60 (d, J = 7.0 Hz, 1H), 7.33-7.28 (m, 5H), 7.22-7.19 (m, 1H), 6.33 (s, 1H), 6.31 (dd, J = 7.0, 2.0 Hz, 1H), 4.36 (s, 2H), 3.56 (s, 2H), 3.12 (t, J = 6.0 Hz, 2H), 2.92 (t, J = 7.8 Hz, 2H), 2.79 (t, J = 8.0 Hz, 2H) |
| 105 | 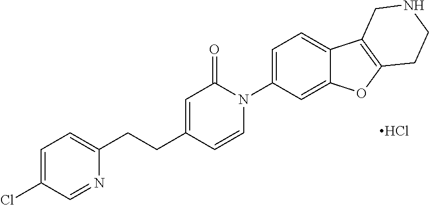 [SMILES: O=c1cc(CCc2ccc(Cl)cn2)ccn1-c1ccc2c3c(oc2c1)CCNC3 ; Cl] | 12 | 406 | ¹H NMR (500 MHz, DMSO-d₆) δ 9.76 (s, 2H), 8.59 (d, J = 2.5 Hz, 1H), 7.90 (dd, J = 8.3, 2.8 Hz, 1H), 7.70-7.69 (m, 2H), 7.59 (d, J = 7.0 Hz, 1H), 7.44 (d, J = 8.5 Hz, 1H), 7.28 (dd, J = 8.5, 2.0 Hz, 1H), 6.32 (s, 1H), 6.28 (dd, J = 7.0, 1.5 Hz, 1H), 4.34 (s, 2H), 3.55- 3.52 (m, 2H), 3.13-3.08 (m, 4H), 2.91 (t, J = 7.3 Hz, 2H) |
| 106 | 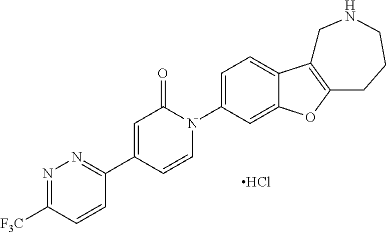 [SMILES: O=c1cc(-c2ccc(C(F)(F)F)nn2)ccn1-c1ccc2c3c(oc2c1)CCCNC3 ; Cl] | 199 | 427 | ¹H NMR (500 MHz, DMSO-d₆) δ 9.32 (s, 2H), 8.71 (d, J = 9.0 Hz, 1H), 8.47 (d, J = 9.0 Hz, 1H), 7.93 (d, J = 7.0 Hz, 1H), 7.83 (d, J = 8.5 Hz, 1H), 7.78 (d, J = 1.5 Hz, 1H), 7.40-7.38 (m, 2H), 7.19 (dd, J = 7.5, 2.0 Hz, 1H), 4.44 (s, 2H), 3.50-3.48 (m, 2H), 3.14- 3.12 (m, 2H), 2.11-2.09 (m, 2H) |
| 107 | 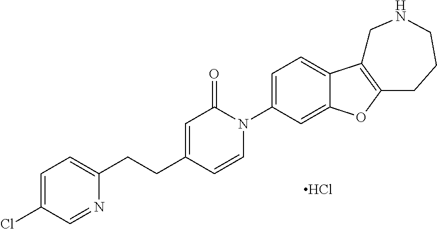 [SMILES: O=c1cc(CCc2ccc(Cl)cn2)ccn1-c1ccc2c3c(oc2c1)CCCNC3 ; Cl] | 4.2 | 420 | ¹H NMR (500 MHz, DMSO-d₆) δ 9.29 (s, 2H), 8.57-8.56 (m, 1H), 7.88-7.86 (m, 1H), 7.77 (dd, J = 8.5, 1.5 Hz, 1H), 7.64-7.63 (m, 1H), 7.58 (dd, J = 7.0, 1.5 Hz, 1H), 7.41 (dd, J = 8.0, 1.0 Hz, 1H), 7.28-7.25 (m, 1H), 6.31- 6.27 (m, 2H), 4.41 (br s, 2H), 3.47 (br s, 2H), 3.12-3.07 (m, 4H), 2.92-2.89 (m, 2H), 2.11-2.08 (m, 2H) |
| 108 | 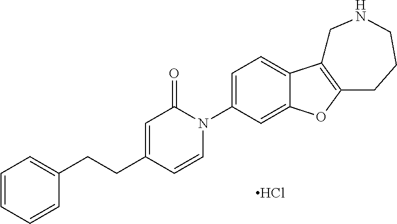 [SMILES: O=c1cc(CCc2ccccc2)ccn1-c1ccc2c3c(oc2c1)CCCNC3 ; Cl] | 3.2 | 385 | ¹H NMR (500 MHz, DMSO-d₆) δ 9.43 (s, 2H), 7.77 (d, J = 8.5 Hz, 1H), 7.64 (d, J = 2.0 Hz, 1H), 7.59 (d, J = 7.0 Hz, 1H), 7.33-7.26 (m, 5H), 7.22- 7.19 (m, 1H), 6.32-6.30 (m, 2H), 4.40 (s, 2H), 3.48-3.46 (m, 2H), 3.12-3.10 (m, 2H), 2.93-2.90 (m, 2H), 2.80- 2.77 (m, 2H), 2.11-2.07 (m, 2H) |
| 109 | 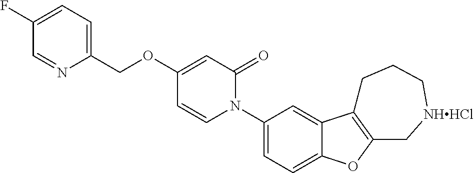 | 12% inhibition @ 1 uM | 406 | ¹H NMR (400 MHz, DMSO-d₆) δ 9.89-9.80 (m, 2H), 8.62 (d, J = 3.2 Hz, 1H), 7.88-7.79 (m, 1H), 7.70- 7.60 (m, 4H), 7.28 (dd, J = 8.8, 2.0 Hz, 1H), 6.15 (dd, J = 7.6, 2.4 Hz, 1H), 6.00 (d, J = 2.8 Hz, 1H), 5.22 (s, 2H), 4.55-4.46 (m, 2H), 3.51-3.41 (m, 2H), 2.85 (t, J = 5.6 Hz, 2H), 2.14-2.03 (m, 2H) |
| 110 | 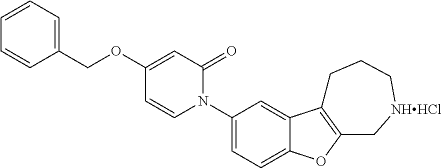 | 12% inhibition @ 1 uM | 387 | ¹H NMR (400 MHz, DMSO-d₆) δ 9.69-9.58 (m, 2H), 7.68-7.58 (m, 3H), 7.50-7.34 (m, 5H), 7.28 (dd, J = 8.8, 1H), 6.12 (dd, J = 7.6, 1H), 5.99 (d, J = 2.8 Hz, 1H), 5.15 (s, 2H), 4.56-4.49 (m, 2H), 3.52-3.42 (m, 2H), 2.85 (t, J = 5.2 Hz, 2H), 2.13- 2.02 (m, 2H) |
| 111 | 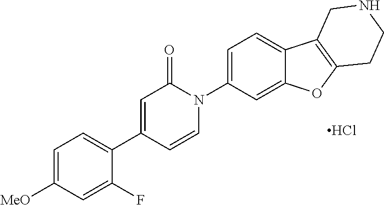 [SMILES: COc1ccc(-c2ccn(-c3ccc4c5c(oc4c3)CCNC5)c(=O)c2)c(F)c1 ; Cl] | 16 | 391 | ¹H NMR (500 MHz, DMSO-d₆) δ 9.63 (s, 2H), 7.79 (d, J = 1.5 Hz, 1H), 7.75-7.72 (m, 2H), 7.61 (t, J = 8.8 Hz, 1H), 7.37 (dd, J = 8.0, 1.5 Hz, 1H), 7.01 (dd, J = 13.3, 2.3 Hz, 1H), 6.93 (dd, J = 8.8, 2.3 Hz, 1H), 6.63 (s, 1H), 6.55-6.54 (m, 1H), 4.37 (s, 2H), 3.84 (s, 3H), 3.57-3.54 (m, 2H), 3.14-3.12 (m, 2H) |
| 112 | 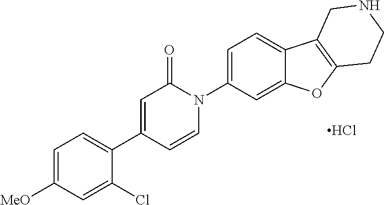 [SMILES: COc1ccc(-c2ccn(-c3ccc4c5c(oc4c3)CCNC5)c(=O)c2)c(Cl)c1 ; Cl] | 6.9 | 407 | ¹H NMR (500 MHz, DMSO-d₆) δ 9.56 (s, 2H), 7.81 (d, J = 1.5 Hz, 1H), 7.75-7.73 (m, 2H), 7.45 (d, J = 8.5 Hz, 1H), 7.38 (dd, J = 8.3, 1.8 Hz, 1H), 7.20 (d, J = 2.5 Hz, 1H), 7.06 (dd, J = 8.5, 2.5 Hz, 1H), 6.50 (d, J = 1.5 Hz, 1H), 6.43 (dd, J = 7.3, 1.8 Hz, 1H), 4.37 (s, 2H), 3.84 (s, 3H), 3.56 (t, J = 5.8 Hz, 2H), 3.13 (t, J = 5.8 Hz, 2H) |
| 113 | 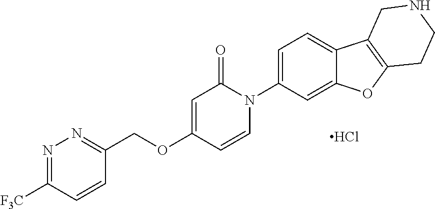 [SMILES: O=c1cc(OCc2ccc(C(F)(F)F)nn2)ccn1-c1ccc2c3c(oc2c1)CCNC3 ; Cl] | 20 | 443 | ¹H NMR (500 MHz, DMSO-d₆) δ 9.56 (s, 2H), 8.38 (d, J = 9.0 Hz, 1H), 8.18 (d, J = 9.0 Hz, 1H), 7.71-7.69 (m, 2H), 7.66 (d, J = 7.5 Hz, 1H), 7.28 (dd, J = 8.3, 1.5 Hz, 1H), 6.22 (dd, J = 7.8, 2.8 Hz, 1H), 6.08 (d, J = 2.5 Hz, 1H), 5.62 (s, 2H), 4.35 (s, 2H), 3.55 (t, J = 6.0 Hz, 2H), 3.12 (t, J = 5.8 Hz, 2H) |
| 114 | 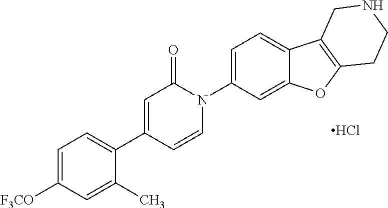 [SMILES: Cc1cc(OC(F)(F)F)ccc1-c1ccn(-c2ccc3c4c(oc3c2)CCNC4)c(=O)c1 ; Cl] | 7.3 | 441 | ¹H NMR (500 MHz, DMSO-d₆) δ 9.64 (br s, 2H), 7.81 (d, J = 1.5 Hz, 1H), 7.76-7.73 (m, 2H), 7.43 (d, J = 8.5 Hz, 1H), 7.40-7.38 (m, 2H), 7.30 (d, J = 8.0 Hz, 1H), 6.46 (d, J = 1.5 Hz, 1H), 6.39 (dd, J = 7.0, 2.0 Hz, 1H), 4.37 (s, 2H), 3.56 (t, J = 5.8 Hz, 2H), 3.14 (s, 2H), 2.39 (s, 3H) |
| 115 | 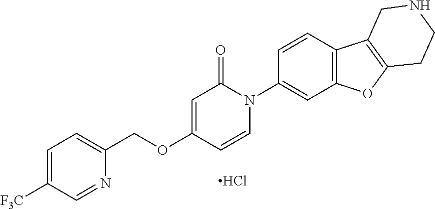 [SMILES: O=c1cc(OCc2ccc(C(F)(F)F)cn2)ccn1-c1ccc2c3c(oc2c1)CCNC3 ; Cl] | 13 | 442 | ¹H NMR (500 MHz, CD₃OD) 8.96 (d, J = 2.1, Hz, 1H), 8.27 (dd, J = 8.3, 2.4 Hz, 1H), 7.86 (d, J = 8.2 Hz, 1H), 7.73 (d, J = 7.6 Hz, 1H), 7.70 (d, J = 8.2 Hz, 1H), 7.65 (d, J = 1.8 Hz, 1H), 7.32 (dd, J = 8.3, 1.8 Hz, 1H), 6.52 (dd, J = 7.6, 2.7 Hz, 1H), 6.25 (d, J = 2.7 Hz, 1H), 5.45 (s, 2H), 4.48 (t, J = 1.9 Hz, 2H), 3.71 (t, J = 6.2 Hz, 2H), 3.22 (t, J = 6.2 Hz, 2H) |
| 116 | 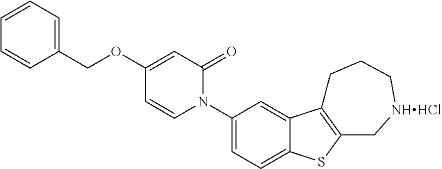 | 37% inhibition @ 1 uM | 403 | ¹H NMR (300 MHz, DMSO-d₆) δ 9.54-9.40 (m, 2H), 8.07 (d, J = 8.4 Hz, 1H), 7.82 (d, J = 1.5 Hz, 1H), 7.65 (d, J = 7.5 Hz, 1H), 7.53-7.32 (m, 6H), 6.15 (dd, J = 7.8, 2.7 Hz, 1H), 6.00 (d, J = 2.4 Hz, 1H), 5.16 (s, 2H), 4.62-4.52 (m, 2H), 3.54-3.41 (m, 2H), 3.17-3.05 (m, 2H), 2.06-1.91 (m, 2H) |
| 117 | 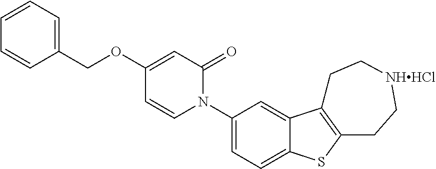 | 103 | 403 | ¹H NMR (400 MHz, DMSO-d₆) δ 9.76-9.64 (m, 2H), 8.01 (d, J = 8.4 Hz, 1H), 7.74 (d, J = 1.2 Hz, 1H), 7.62 (d, J = 7.6 Hz, 1H), 7.52-7.34 (m, 5H), 7.31 (dd, J = 8.4, 1.2 Hz, 1H), 6.14 (dd, J = 7.6, 2.4 Hz, 1H), 6.01 (d, J = 2.4 Hz, 1H), 5.16 (s, 2H), 3.42- 3.21 (m, 8H) |
| 118 | 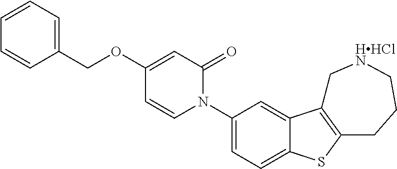 | 7.7% inhibition @ 1 uM | 403 | ¹H NMR (400 MHz, DMSO-d₆) δ 9.15 (br s, 2H), 8.04 (d, J = 8.4 Hz, 1H), 7.94 (d, J = 2.0 Hz, 1H), 7.59 (d, J = 7.6 Hz, 1H), 7.49-7.32 (m, 6H), 6.15 (dd, J = 7.6, 2.8 Hz, 1H), 6.00 (d, J = 2.8 Hz, 1H), 5.17 (s, 2H), 4.54 (s, 2H), 3.48 (s, 2H), 3.19-3.16 (m, 2H), 2.01 (br s, 2H) |
| 119 | 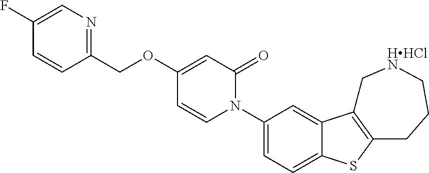 | −2.6% inhibition @ 1 uM | 422 | ¹H NMR (400 MHz, DMSO-d₆) δ 9.29 (br s, 2H), 8.62 (d, J = 3.2 Hz, 1H), 8.03 (d, J = 8.4 Hz, 1H), 7.94 (d, J = 2.0 Hz, 1H), 7.86-7.81 (m, 1H), 7.68-7.61 (m, 2H), 7.33 (dd, J = 8.4, 2.0 Hz, 1H), 6.18 (dd, J = 7.6, 2.8 Hz, 1H), 6.00 (d, J = 2.8 Hz, 1H), 5.23 (s, 2H), 4.52 (br s, 2H), 3.47 (br s, 2H), 3.19-3.16 (m, 2H), 2.02 (br s, 2H) |
| 120 | 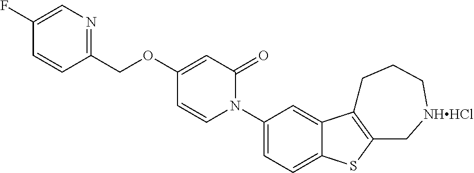 | 1.3% inhibition @ 1 uM | 422 | ¹H NMR (300 MHz, DMSO-d₆) δ 9.35 (br s, 2H), 8.62 (d, J = 3.0 Hz, 1H), 8.06 (d, J = 8.7 Hz, 1H), 7.86- 7.79 (m, 2H), 7.68-7.64 (m, 2H), 7.36 (dd, J = 8.4, 1.8 Hz, 1H), 6.16 (dd, J = 7.8, 2.7 Hz, 1H), 6.00 (d, J = 2.7 Hz, 1H), 5.23 (s, 2H), 4.58 (s, 2H), 3.48 (d, J = 1.8 Hz, 2H), 3.12-3.09 (m, 2H), 1.98 (br s, 2H) |
| 121 | 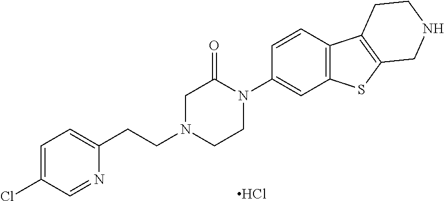 [SMILES: O=C1CN(CCc2ccc(Cl)cn2)CCN1c1ccc2c3c(sc2c1)CNCC3 ; Cl] | 44 | 427 | ¹H NMR (500 MHz, CD₃OD) δ 8.53 (d, J = 2.2 Hz, 1H), 7.91 (d, J = 1.5 Hz, 1H), 7.84-7.82 (dd, J = 8.3, 2.5 Hz, 1H), 7.80 (d, J = 8.5 Hz, 1H), 7.43-7.42 (m, 2H), 4.56 (s, 2H), 3.99- 3.90 (m, 2H), 3.88-3.74 (m, 2H), 3.66 (t, J = 6.2 Hz, 2H), 3.54-3.36 (m, 4H), 3.27-3.21 (m, 2H), 3.18 (t, J = 6.2 Hz, 2H) |
| 122 | 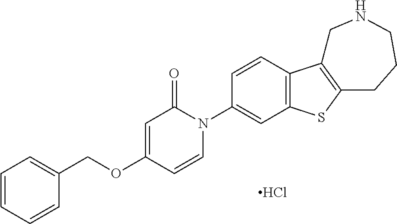 [SMILES: O=c1cc(OCc2ccccc2)ccn1-c1ccc2c3c(sc2c1)CCCNC3 ; Cl] | 3.3 | 403 | ¹H NMR (500 MHz, DMSO-d₆) δ 9.19 (s, 2H), 8.00 (d, J = 1.8 Hz, 1H), 7.95 (d, J = 8.6 Hz, 1H), 7.61 (d, J = 7.6 Hz, 1H), 7.47-7.36 (m, 6H), 6.14-6.12 (dd, J = 7.7, 2.7 Hz, 1H), 5.99 (d, J = 2.7 Hz, 1H), 5.15 (s, 2H), 4.58-4.52 (m, 2H), 3.52-3.45 (m, 2H), 3.20-3.13 (m, 2H), 2.06-1.97 (m, 2H) |
| 123 | 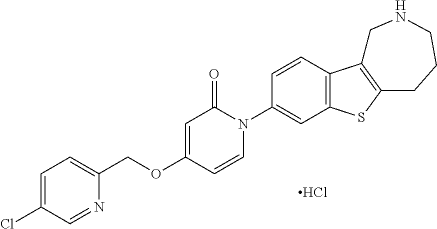 [SMILES: O=c1cc(OCc2ccc(Cl)cn2)ccn1-c1ccc2c3c(sc2c1)CCCNC3 ; Cl] | 2.9 | 438 | ¹H NMR (500 MHz, DMSO-d₆) δ 9.20 (s, 2H), 8.33 (d, J = 2.4 Hz, 1H), 8.04-8.02 (dd, J = 8.3, 2.5 Hz, 1H), 8.00 (d, J = 1.9 Hz, 1H), 7.95 (d, J = 8.7 Hz, 1H), 7.67-7.60 (m, 2H), 7.41- 7.37 (dd, J = 8.6, 1.9 Hz, 1H), 6.18- 6.16 (dd, J = 7.6, 2.7 Hz, 1H), 5.97 (d, J = 2.7 Hz, 1H), 5.23 (s, 2H), 4.57- 4.52 (m, 2H), 3.52-3.46 (m, 2H), 3.20-3.13 (m, 2H), 2.06-1.97 (m, 2H) |
| 124 | 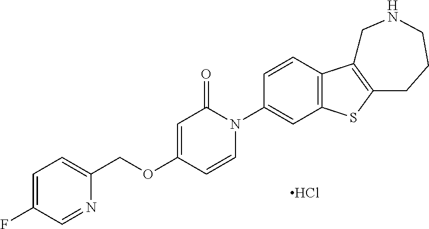 [SMILES: O=c1cc(OCc2ccc(F)cn2)ccn1-c1ccc2c3c(sc2c1)CCCNC3 ; Cl] | 3.7 | 422 | ¹H NMR (500 MHz, DMSO-d₆) δ 9.26 (s, 2H), 8.61 (d, J = 2.9 Hz, 1H), 8.00 (d, J = 1.9 Hz, 1H), 7.95 (d, J = 8.6 Hz, 1H), 7.84-7.80 (td, J = 8.7, 2.9 Hz, 1H), 7.67-7.62 (m, 2H), 7.41- 7.37 (dd, J = 8.6, 1.9 Hz, 1H), 6.17- 6.15 (dd, J = 7.6, 2.7 Hz, 1H), 5.99 (d, J = 2.7 Hz, 1H), 5.22 (s, 2H), 4.57- 4.51 (m, 2H), 3.52-3.46 (m, 2H), 3.18-3.16 (m, 2H), 2.05-1.98 (m, 2H) |
| 125 | 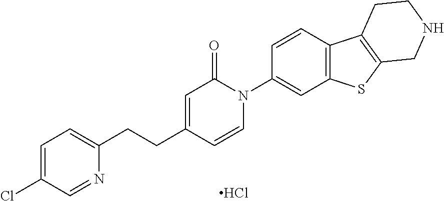 [SMILES: O=c1cc(CCc2ccc(Cl)cn2)ccn1-c1ccc2c3c(sc2c1)CNCC3 ; Cl] | 10 | 421 | ¹H NMR (500 MHz, DMSO-d₆) δ 9.74 (s, 2H), 8.57 (d, J = 2.5 Hz, 1H), 8.07 (d, J = 1.8 Hz, 1H), 7.88-7.86 (dd, J = 8.3, 2.6 Hz, 1H), 7.82 (d, J = 8.5 Hz, 1H), 7.60 (d, J = 7.0 Hz, 1H), 7.43-7.40 (m, 2H), 6.32-6.28 (m, 2H), 4.51-4.45 (m, 2H), 3.54-3.47 (m, 2H), 3.12-3.05 (m, 4H), 2.92- 2.89 (m, 2H) |
| 126 | 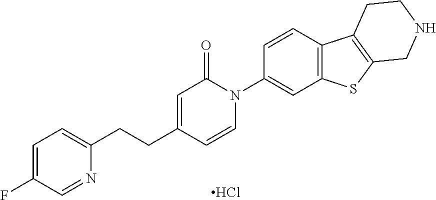 [SMILES: O=c1cc(CCc2ccc(F)cn2)ccn1-c1ccc2c3c(sc2c1)CNCC3 ; Cl] | 20 | 406 | ¹H NMR (500 MHz, DMSO-d₆) δ 9.70 (s, 2H), 8.52 (d, J = 2.9 Hz, 1H), 8.07 (d, J = 1.8 Hz, 1H), 7.82 (d, J = 8.5 Hz, 1H), 7.71-7.65 (td, J = 8.7, 3.0 Hz, 1H), 7.60 (d, J = 7.0 Hz, 1H), 7.46-7.41 (m, 2H), 6.32-6.28 (m, 2H), 4.52-4.46 (m, 2H), 3.54-3.47 (m, 2H), 3.12-3.04 (m, 4H), 2.94- 2.89 (m, 2H) |
| 127 | 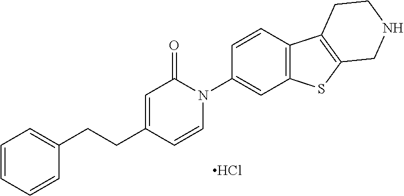 [SMILES: O=c1cc(CCc2ccccc2)ccn1-c1ccc2c3c(sc2c1)CNCC3 ; Cl] | 6.2 | 387 | ¹H NMR (500 MHz, DMSO-d₆) δ 9.78 (s, 2H), 8.07 (d, J = 1.8 Hz, 1H), 7.82 (d, J = 8.4 Hz, 1H), 7.62 (d, J = 6.9 Hz, 1H), 7.46-7.41 (dd, J = 8.5, 1.8 Hz, 1H), 7.33-7.26 (m, 4H), 7.23- 7.18 (m, 1H), 6.33-6.31 (m, 2H), 4.52-4.45 (m, 2H), 3.53-3.45 (m, 2H), 3.09-3.07 (m, 2H), 2.94-2.88 (m, 2H), 2.80-2.72 (m, 2H) |
| 128 | 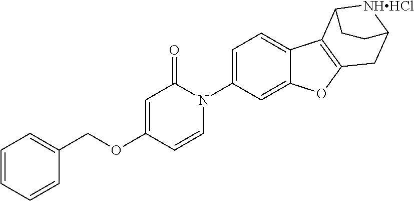 | 11 | 399 | ¹H NMR (300 MHz, CD₃OD) δ 7.74 (d, J = 7.6 Hz, 1H), 7.65-7.52 (m, 2H), 7.50-7.24 (m, 6H), 6.35-6.25 (m, 1H), 6.11 (br s, 1H), 5.24 (s, 1H), 5.17 (s, 2H), 4.62-4.53 (m, 1H), 3.60- 3.48 (m, 1H), 3.07 (d, J = 17.6 Hz, 1H), 2.58-2.32 (m, 3H), 2.11-1.97 (m, 1H) |
| 129 | 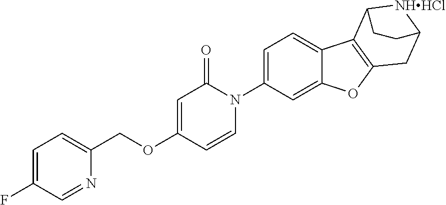 | 14 | 418 | ¹H NMR (400 MHz, DMSO-d₆) δ 10.07-9.97 (m, 1H), 9.53 (d, J = 10.0 Hz, 1H), 8.62 (d, J = 2.8 Hz, 1H), 7.87-7.75 (m, 2H), 7.69-7.59 (m, 3H), 7.27 (dd, J = 8.4 Hz, 1.6 Hz, 1H), 6.16 (dd, J = 7.6 Hz, 2.4 Hz, 1H), 5.99 (d, J = 2.8 Hz, 1H), 5.27-5.19 (m, 3H), 4.53 (m, 1H), 3.48 (dd, J = 17.6 Hz, 4.4 Hz, 1H), 3.00 (d, J = 16.8 Hz, 1H), 2.37-2.50 (m, 2H), 2.17 (t, J = 10.0 Hz, 1H), 1.93-1.80 (m, 1H) |

As compounds that bind strongly to MCH-1, compounds of formula I are expected to be effective in reducing obesity and in treating the other diseases and disorders described herein.

The present invention is not limited to the compounds found in the above examples, and many other compounds falling within the scope of the invention may also be prepared using the procedures set forth in the above synthetic schemes. The preparation of additional compounds of formula (I) using these methods will be apparent to one of ordinary skill in the chemical arts.

Although particular embodiments have been depicted and described in detail herein, it will be apparent to those skilled in the relevant art that various modifications, additions, substitutions, and the like can be made without departing from the spirit of the invention and these are therefore considered to be within the scope of the invention as defined in the claims which follow.

What is claimed:

1. A compound of formula (I): 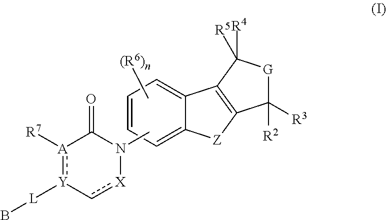
wherein
R²-R⁵ and R⁹-R¹² are each, independently, selected from the group consisting of H, halogen, —OR¹³, —NR¹³R¹⁴, —NR¹³C(O)R¹⁴, —NR¹³C(O)₂R¹⁴, —NR¹⁵C(O)NR¹⁴R¹⁵, —S(O)_{q}R¹⁴, —CN, —C(O)R¹⁴, —C(O)NR¹³R¹⁴, C₁-C₆ alkyl, C₂-C₆ alkenyl, C₂-C₆ alkynyl, C₃-C₆ cycloalkyl, C₄-C₇ cycloalkylalkyl, heterocyclyl, aryl, and heteroaryl, wherein each of C₁-C₆ alkyl, C₂-C₆ alkenyl, C₂-C₆ alkynyl, C₃-C₆ cycloalkyl, C₄-C₇ cycloalkylalkyl, heterocyclyl, aryl, and heteroaryl is optionally substituted with from 1 to 3 substituents independently selected at each occurrence thereof from C₁-C₃ alkyl, halogen, —CN, —OR¹⁶, —NR¹⁶R¹⁷, and phenyl which is optionally substituted 1-3 times with halogen, C₁-C₄ alkyl, C₁-C₄ haloalkyl, C₁-C₄ alkoxy, —CN, —OR¹⁶, or —NR¹⁶R¹⁷; or R² and R³ or R⁴ and R⁵ can combine to form an oxo, thio, imine, cycloalkyl, or heterocycle group containing from 1 to 5 heteroatoms selected from the group consisting of oxygen, nitrogen, and sulfur; or any one of R², R³, R⁴, or R⁵ can combine with any one of R⁹, R¹⁰, R¹¹, or R¹² to form —(CH₂)ᵣ—;
R⁶ is independently selected at each location from the group consisting of H, halogen, —OR¹³, —NR¹³C(O)R¹⁴, —NR¹³C(O)₂R¹⁴, —NR¹⁵C(O)NR¹⁴R¹⁵, —S(O)_{q}R¹⁴, —CN, —C(O)R¹⁴, —C(O)NR¹³R¹⁴, C₁-C₆ alkyl, C₂-C₆ alkenyl, C₂-C₆ alkynyl, C₃-C₆ cycloalkyl, C₄-C₇ cycloalkylalkyl, heterocyclyl, aryl, and heteroaryl, wherein each of C₁-C₆ alkyl, C₂-C₆ alkenyl, C₂-C₆ alkynyl, C₃-C₆ cycloalkyl, C₄-C₇ cycloalkylalkyl, heterocyclyl, aryl, and heteroaryl is optionally substituted with from 1 to 3 substituents independently selected at each occurrence thereof from C₁-C₃ alkyl, halogen, —CN, —OR⁶, —NR¹⁶R¹⁷, and phenyl which is optionally substituted 1-3 times with halogen, C₁-C₄ alkyl, C₁-C₄ haloalkyl, C₁-C₄ alkoxy, —CN, —OR¹⁶, or NR¹⁶R¹⁷;
R⁷ is optionally present and, if present, is selected from the group consisting of H, halogen, —OR¹³, —NR¹³R¹⁴, —NR¹³C(O)R¹⁴, —NR¹³C(O)₂R¹⁴, —NR¹⁵C(O)NR¹⁴R¹⁵, —S(O)_{q}R¹⁴, —CN, —C(O)R¹⁴, —C(O)NR¹³R¹⁴, C₁-C₆ alkyl, C₂-C₆ alkenyl, C₂-C₆ alkynyl, C₃-C₆ cycloalkyl, C₄-C₇ cycloalkylalkyl, heterocyclyl, aryl, and heteroaryl, wherein each of C₁-C₆ alkyl, C₂-C₆ alkenyl, C₂-C₆ alkynyl, C₃-C₆ cycloalkyl, C₄-C₇ cycloalkylalkyl, heterocyclyl, aryl, and heteroaryl is optionally substituted with from 1 to 3 substituents independently selected at each occurrence thereof from C₁-C₃ alkyl, halogen, —CN, —OR¹⁶, —NR¹⁶R¹⁷, and phenyl which is optionally substituted 1-3 times with halogen, C₁-C₄ alkyl, C₁-C₄ haloalkyl, C₁-C₄ alkoxy, —CN, —OR¹⁶, or —NR¹⁶R¹⁷;
R⁸ is selected from the group consisting of H, —S(O)_{q}R¹⁴, —C(O)R¹⁴, —C(O)NR¹³R¹⁴, C₁-C₆ alkyl, C₂-C₆ alkenyl, C₂-C₆ alkynyl, C₃-C₆ cycloalkyl, C₄-C₇ cycloalkylalkyl, heterocyclyl, and heteroaryl, wherein each of C₁-C₆ alkyl, C₂-C₆ alkenyl, C₂-C₆ alkynyl, C₃-C₆ cycloalkyl, C₄-C₇ cycloalkylalkyl, heterocyclyl, and heteroaryl is optionally substituted with from 1 to 3 substituents independently selected at each occurrence thereof from C₁-C₃ alkyl, halogen, —CN, —OR¹⁶, —NR¹⁶R¹⁷, and phenyl which is optionally substituted 1-3 times with halogen, C₁-C₄ alkyl, C₁-C₄ haloalkyl, C₁-C₄ alkoxy, —CN, —OR¹⁶, or NR¹⁶R¹⁷; or
R⁸ and one of R², R³, R⁴, and R⁵ can combine to form a 3- to 7-membered heterocycle, wherein the 3- to 7-membered heterocycle includes from 1 to 2 heteroatoms selected from the group consisting of N, O, and S and is optionally substituted with from 1 to 10 substituents independently selected at each occurrence thereof from H, halogen, —OR¹³, —NR¹³R¹⁴, —NR¹³C(O)R¹⁴, —NR¹³C(O)₂R¹⁴, —NR¹⁵C(O)NR¹⁴R¹⁵, —S(O)_{q}R¹⁴, —CN, —C(O)R¹⁴, —C(O)NR¹³R¹⁴, C₁-C₆ alkyl, C₂-C₆ alkenyl, C₂-C₆ alkynyl, C₃-C₆ cycloalkyl, C₄-C₇ cycloalkylalkyl, heterocyclyl, aryl, and heteroaryl, wherein each of C₁-C₆ alkyl, C₂-C₆ alkenyl, C₂-C₆ alkynyl, C₃-C₆ cycloalkyl, C₄-C₇ cycloalkylalkyl, heterocyclyl, aryl, and heteroaryl is optionally substituted with from 1 to 3 substituents independently selected at each occurrence thereof from C₁-C₃ alkyl, halogen, —CN, —OR¹⁶, —NR¹⁶R¹⁷, and phenyl which is optionally substituted 1-3 times with halogen, C₁-C₄ alkyl, C₁-C₄ haloalkyl, C₁-C₄ alkoxy, —CN, —OR¹⁶, or —NR¹⁶R¹⁷;
R¹³ is H, C₁-C₄ alkyl, C₁-C₄ haloalkyl, C₁-C₄ alkoxyalkyl, C₃-C₆ cycloalkyl, C₄-C₇ cycloalkylalkyl, —C(O)R¹⁵, phenyl, or benzyl, wherein phenyl or benzyl is optionally substituted 1 to 3 times with halogen, cyano, C₁-C₄ alkyl, C₁-C₄ haloalkyl, or C₁-C₄ alkoxy;
R¹⁴ is H, C₁-C₄ alkyl, C₁-C₄ haloalkyl, C₁-C₄ alkoxyalkyl, C₃-C₆ cycloalkyl, C₄-C₇ cycloalkylalkyl, phenyl, or benzyl, wherein phenyl or benzyl is optionally substituted 1 to 3 times with halogen, cyano, C₁-C₄ alkyl, C₁-C₄ haloalkyl, or C₁-C₄ alkoxy;
R¹⁵ is C₁-C₄ alkyl, C₁-C₄ haloalkyl, or phenyl;
R¹⁶ and R¹⁷ are each independently H, C₁-C₄ alkyl, C₁-C₄ haloalkyl, C₁-C₄ alkoxyalkyl, C₃-C₆ cycloalkyl, C₄-C₇ cycloalkylalkyl, —C(O)R¹⁵, phenyl, or benzyl, wherein phenyl or benzyl is optionally substituted from 1 to 3 times with a substituent selected independently at each occurrence thereof from the group consisting of halogen, cyano, C₁-C₄ alkyl, C₁-C₄ haloalkyl, and C₁-C₄ alkoxy;
R¹⁸ is selected from the group consisting of H, halogen, —OR¹³, —NR¹³R¹⁴—NR¹³C(O)R¹⁴, —NR¹³C(O)₂R¹⁴, —NR¹⁴C(O)NR¹⁴R¹⁵, —S(O)_{q}R¹⁴, —CN, —C(O)R¹⁴, —C(O)NR¹³R¹⁴, C₁-C₆ alkyl, C₂-C₆ alkenyl, C₂-C₆ alkynyl, C₃-C₆ cycloalkyl, C₄-C₇ cycloalkylalkyl, heterocyclyl, aryl, and heteroaryl, wherein each of C₁-C₆ alkyl, C₂-C₆ alkenyl, C₂-C₆ alkynyl, C₃-C₆ cycloalkyl, C₄-C₇ cycloalkylalkyl, heterocyclyl, aryl, and heteroaryl is optionally substituted with from 1 to 3 substituents independently selected at each occurrence thereof from C₁-C₃ alkyl, halogen, —CN, —OR¹⁶, —NR¹⁶R¹⁷, and phenyl which is optionally substituted 1-3 times with halogen, C₁-C₄ alkyl, C₁-C₄ haloalkyl, C₁-C₄ alkoxy, —CN, —OR⁸, or —NR⁸R⁹;
G is —NR⁸—CR⁹R¹⁰—, —CR⁹R¹⁰—NR⁸—, —NR⁸—CR⁹R¹⁰—CR¹¹R¹²—, —CR⁹R¹⁰—NR⁸—CR¹¹R¹²—, or —CR⁹R¹⁰—CR¹¹R¹²—NR⁸—;
X is CR¹⁸, C(R¹⁸)₂, N, or NR¹⁸;
Y is CR¹⁸, C, or N;
Z is O or S;
L is —(CH₂)ₚ—O—, —(CH₂)ₚ—, —CH═CH—, or a bond;
A is C, CH, or N;
B is aryl, heteroaryl, heterocyclyl, or cycloalkyl, wherein each of the aryl, heteroaryl, heterocyclyl, or cycloalkyl is optionally substituted with from 1 to 3 substituents selected from the group consisting of H, alkoxy, —S-alkyl, optionally substituted C₁-C₆ alkyl, halogen, —CF₃, —OCF₃, and —CN;
n is 0, 1, 2, or 3;
p is from 1 to 4;
q is 0, 1, or 2;
r is from 1 to 4; and [symbol]
represents an optional double bond,
or an oxide thereof, a pharmaceutically acceptable salt thereof, a solvate thereof, or prodrug thereof.

2. The compound according to claim 1, wherein G is —NR⁸—CR⁹R¹⁰—.

3. The compound according to claim 1, wherein G is —CR⁹R¹⁰—NR⁸—.

4. The compound according to claim 1, wherein G is —NR⁸—CR⁹R¹⁰—CR¹¹R¹²—.

5. The compound according to claim 1, wherein G is —CR⁹R¹⁰—NR⁸—CR¹¹R¹²—.

6. The compound according to claim 1, wherein G is —CR⁹R¹⁰—CR¹¹R¹²—NR⁸—.

7. The compound according to claim 1, wherein R² to R⁵ are each independently selected from the group consisting of H and optionally substituted C₁-C₆ alkyl.

8. The compound according to claim 1, wherein R⁶ is H, halogen, or optionally substituted C₁-C₆ alkyl.

9. The compound according to claim 1, wherein R⁷ is H, halogen, or optionally substituted C₁-C₆ alkyl.

10. The compound according to claim 1, wherein R⁸ is H or C₁-C₆ alkyl and R⁹-R¹² are H.

11. The compound according to claim 1, wherein R⁸ and one of R², R³, R⁴, and R⁵ combine to form a 3- to 7-membered heterocycle.

12. The compound according to claim 1, wherein any one of R², R³, R⁴, or R⁵ combine with any one of R⁹, R¹⁰, R¹, or R¹² to form —(CH₂)ᵣ— and r is from 1 to 4.

13. The compound according to claim 1, wherein R⁸ is H or C₁-C₆ alkyl.

14. The compound according to claim 1, wherein R⁸ is —C(O)R¹⁴.

15. The compound according to claim 1, wherein Z is O.

16. The compound according to claim 1, wherein Z is S.

17. The compound according to claim 1, wherein X is N, CH, or CH₂.

18. The compound according to claim 1, wherein Y is N or C.

19. The compound according to claim 1, wherein L is a bond.

20. The compound according to claim 1, wherein L is —CH₂—O—.

21. The compound according to claim 1, wherein L is —CH₂—CH₂—.

22. The compound according to claim 1, wherein B is phenyl or pyridinyl.

23. The compound according to claim 1, wherein B is unsubstituted.

24. The compound according to claim 1, wherein B is substituted with at least one substituent selected from trifluoromethyl, chloro, fluoro, and methyl.

25. The compound according to claim 1, wherein B is selected from the group consisting of phenyl, 4-(trifluoromethyl)phenyl, pyridin-2-yl, 5-(trifluoromethyl)pyridin-2-yl, 5-fluoro-pyridin-2-yl, 6-methylpyridin-3-yl, 6-(trifluoromethyl)pyridin-2-yl, 6-(trifluoromethyl)pyridin-3-yl, 5-chloro-pyridin-2-yl, and 4-chloro-phenyl.

26. The compound according to claim 1, wherein B is selected from the group consisting of 6-(trifluoromethyl)pyridazin-3-yl, 2-fluoro-4-methoxyphenyl, 2-chloro-4-methoxyphenyl, and 2-methyl-4-(trifluoromethoxy)phenyl.

27. The compound according to claim 1, wherein the compound has the structure:

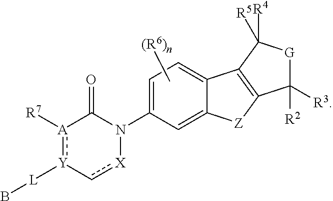

28. The compound according to claim 1, wherein the compound is selected from the group consisting of

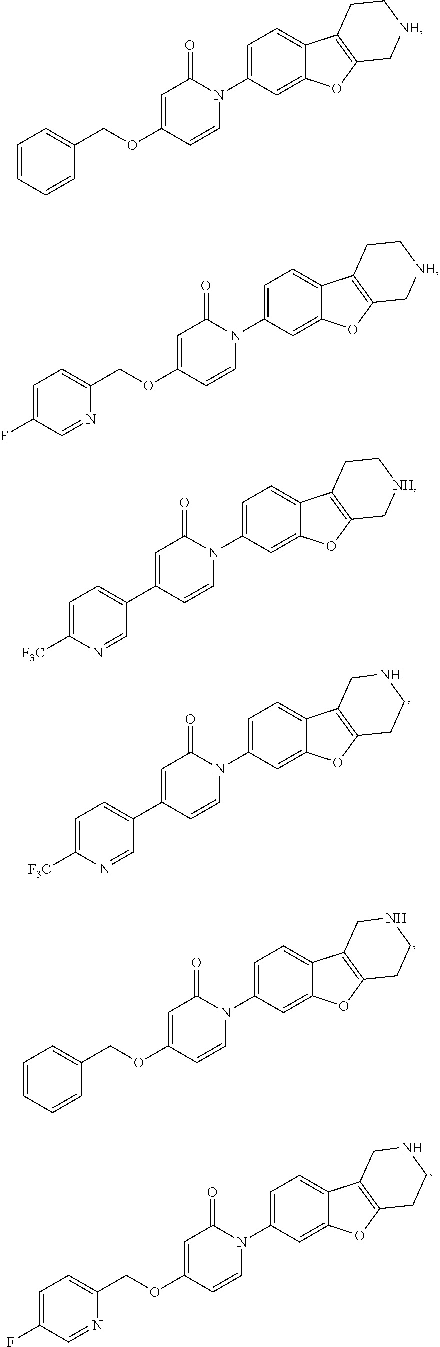 [SMILES: O=c1cc(OCc2ccccc2)ccn1-c1ccc2c3c(oc2c1)CNCC3 ; O=c1cc(OCc2ccc(F)cn2)ccn1-c1ccc2c3c(oc2c1)CNCC3 ; O=c1cc(-c2ccc(C(F)(F)F)nc2)ccn1-c1ccc2c3c(oc2c1)CNCC3 ; O=c1cc(-c2ccc(C(F)(F)F)nc2)ccn1-c1ccc2c3c(oc2c1)CCNC3 ; O=c1cc(OCc2ccccc2)ccn1-c1ccc2c3c(oc2c1)CCNC3 ; O=c1cc(OCc2ccc(F)cn2)ccn1-c1ccc2c3c(oc2c1)CCNC3]

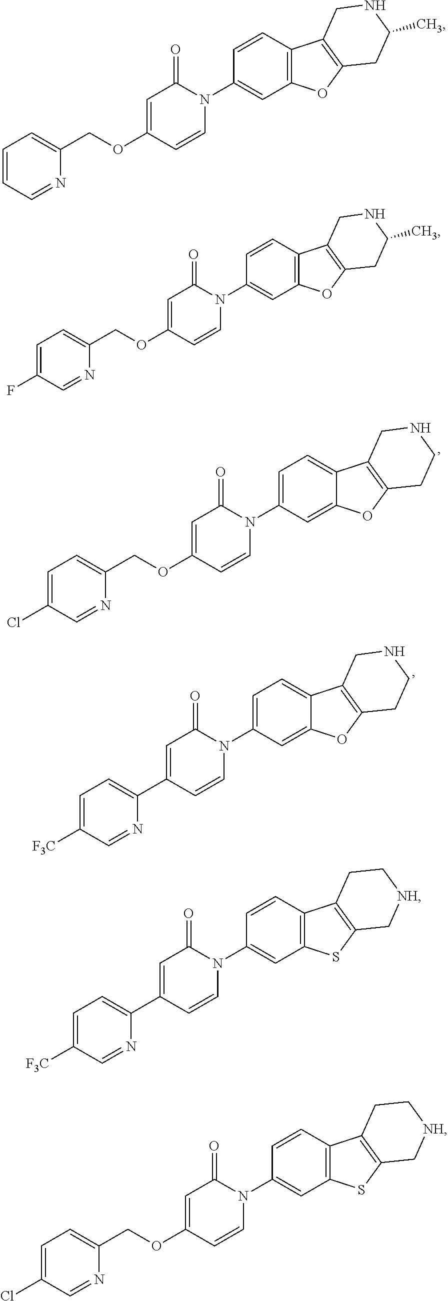 [SMILES: C[C@@H]1Cc2oc3cc(-n4ccc(OCc5ccccn5)cc4=O)ccc3c2CN1 ; C[C@@H]1Cc2oc3cc(-n4ccc(OCc5ccc(F)cn5)cc4=O)ccc3c2CN1 ; O=c1cc(OCc2ccc(Cl)cn2)ccn1-c1ccc2c3c(oc2c1)CCNC3 ; O=c1cc(-c2ccc(C(F)(F)F)cn2)ccn1-c1ccc2c3c(oc2c1)CCNC3 ; O=c1cc(-c2ccc(C(F)(F)F)cn2)ccn1-c1ccc2c3c(sc2c1)CNCC3 ; O=c1cc(OCc2ccc(Cl)cn2)ccn1-c1ccc2c3c(sc2c1)CNCC3]

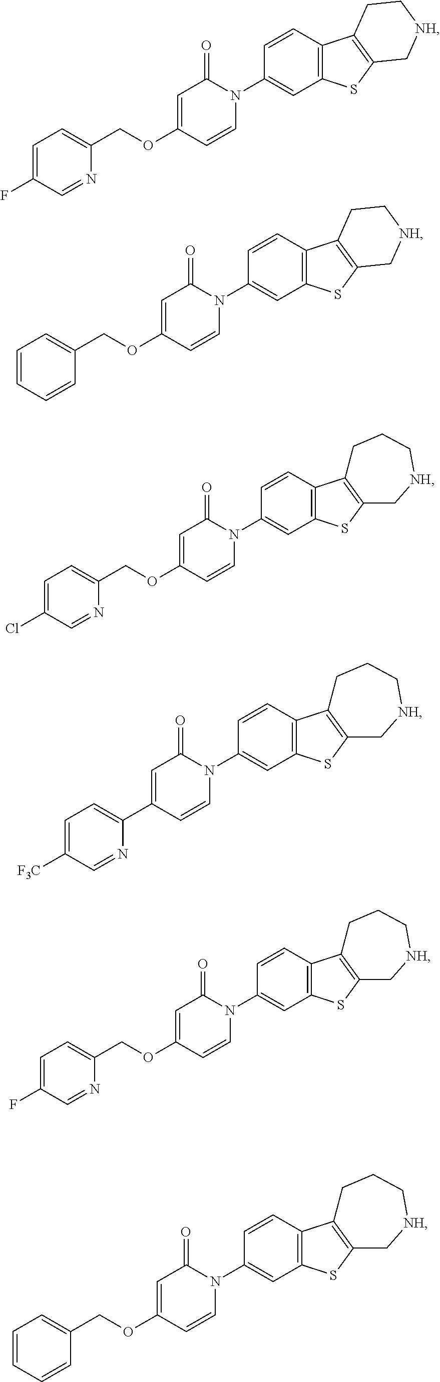 [SMILES: O=c1cc(OCc2ccc(F)cn2)ccn1-c1ccc2c3c(sc2c1)CNCC3 ; O=c1cc(OCc2ccccc2)ccn1-c1ccc2c3c(sc2c1)CNCC3 ; O=c1cc(OCc2ccc(Cl)cn2)ccn1-c1ccc2c3c(sc2c1)CNCCC3 ; O=c1cc(-c2ccc(C(F)(F)F)cn2)ccn1-c1ccc2c3c(sc2c1)CNCCC3 ; O=c1cc(OCc2ccc(F)cn2)ccn1-c1ccc2c3c(sc2c1)CNCCC3 ; O=c1cc(OCc2ccccc2)ccn1-c1ccc2c3c(sc2c1)CNCCC3]

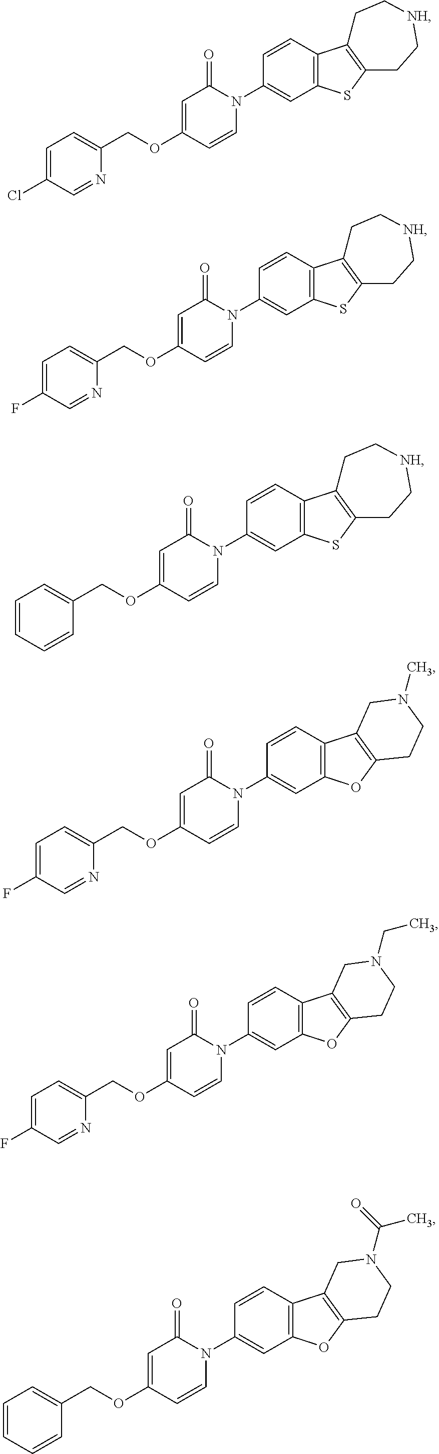 [SMILES: O=c1cc(OCc2ccc(Cl)cn2)ccn1-c1ccc2c3c(sc2c1)CCNCC3 ; O=c1cc(OCc2ccc(F)cn2)ccn1-c1ccc2c3c(sc2c1)CCNCC3 ; O=c1cc(OCc2ccccc2)ccn1-c1ccc2c3c(sc2c1)CCNCC3 ; CN1CCc2oc3cc(-n4ccc(OCc5ccc(F)cn5)cc4=O)ccc3c2C1 ; CCN1CCc2oc3cc(-n4ccc(OCc5ccc(F)cn5)cc4=O)ccc3c2C1 ; CC(=O)N1CCc2oc3cc(-n4ccc(OCc5ccccc5)cc4=O)ccc3c2C1]

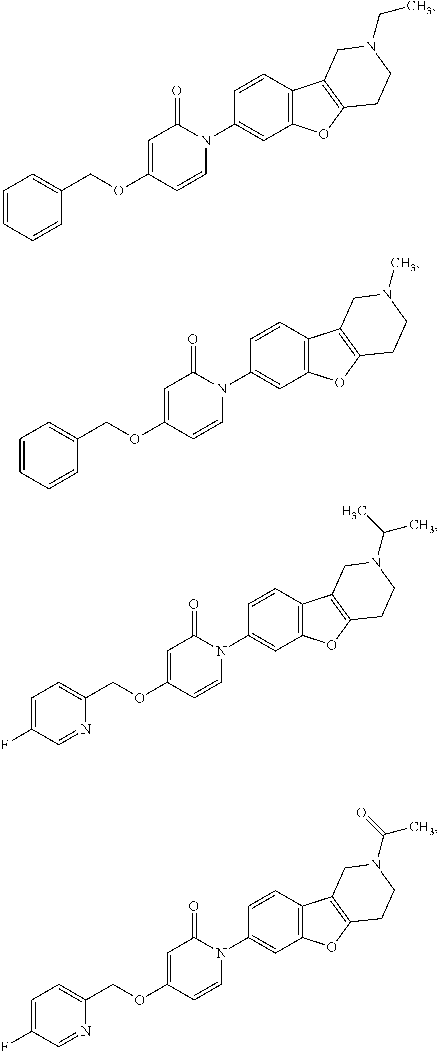 [SMILES: CCN1CCc2oc3cc(-n4ccc(OCc5ccccc5)cc4=O)ccc3c2C1 ; CN1CCc2oc3cc(-n4ccc(OCc5ccccc5)cc4=O)ccc3c2C1 ; CC(C)N1CCc2oc3cc(-n4ccc(OCc5ccc(F)cn5)cc4=O)ccc3c2C1 ; CC(=O)N1CCc2oc3cc(-n4ccc(OCc5ccc(F)cn5)cc4=O)ccc3c2C1]

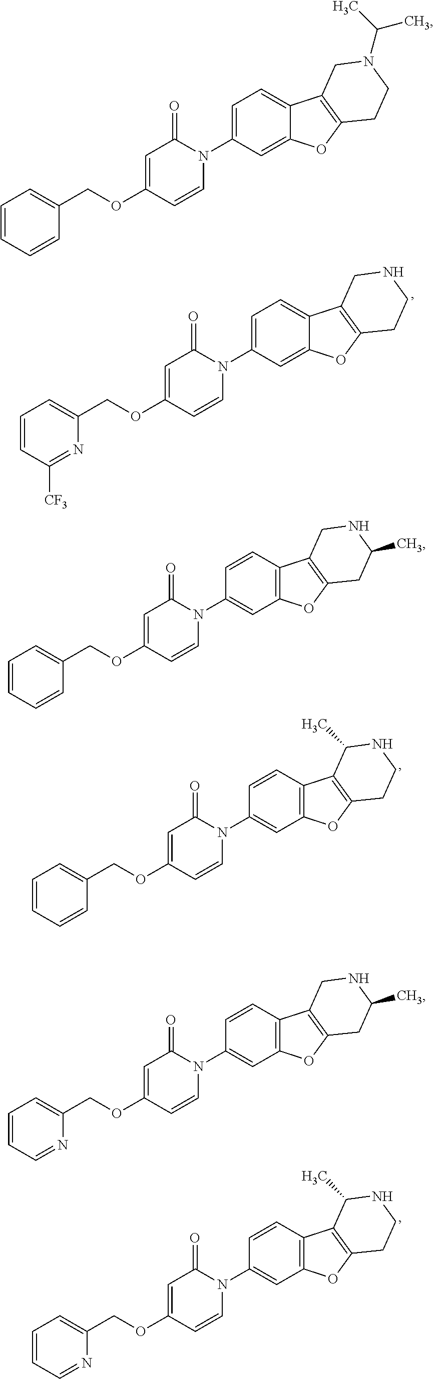 [SMILES: CC(C)N1CCc2oc3cc(-n4ccc(OCc5ccccc5)cc4=O)ccc3c2C1 ; O=c1cc(OCc2cccc(C(F)(F)F)n2)ccn1-c1ccc2c3c(oc2c1)CCNC3 ; C[C@H]1Cc2oc3cc(-n4ccc(OCc5ccccc5)cc4=O)ccc3c2CN1 ; C[C@@H]1NCCc2oc3cc(-n4ccc(OCc5ccccc5)cc4=O)ccc3c21 ; C[C@H]1Cc2oc3cc(-n4ccc(OCc5ccccn5)cc4=O)ccc3c2CN1 ; C[C@@H]1NCCc2oc3cc(-n4ccc(OCc5ccccn5)cc4=O)ccc3c21]

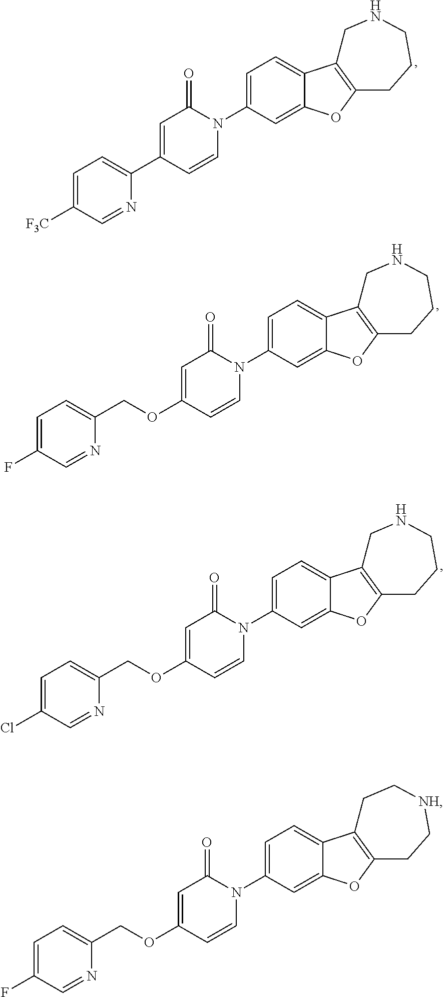 [SMILES: O=c1cc(-c2ccc(C(F)(F)F)cn2)ccn1-c1ccc2c3c(oc2c1)CCCNC3 ; O=c1cc(OCc2ccc(F)cn2)ccn1-c1ccc2c3c(oc2c1)CCCNC3 ; O=c1cc(OCc2ccc(Cl)cn2)ccn1-c1ccc2c3c(oc2c1)CCCNC3 ; O=c1cc(OCc2ccc(F)cn2)ccn1-c1ccc2c3c(oc2c1)CCNCC3]

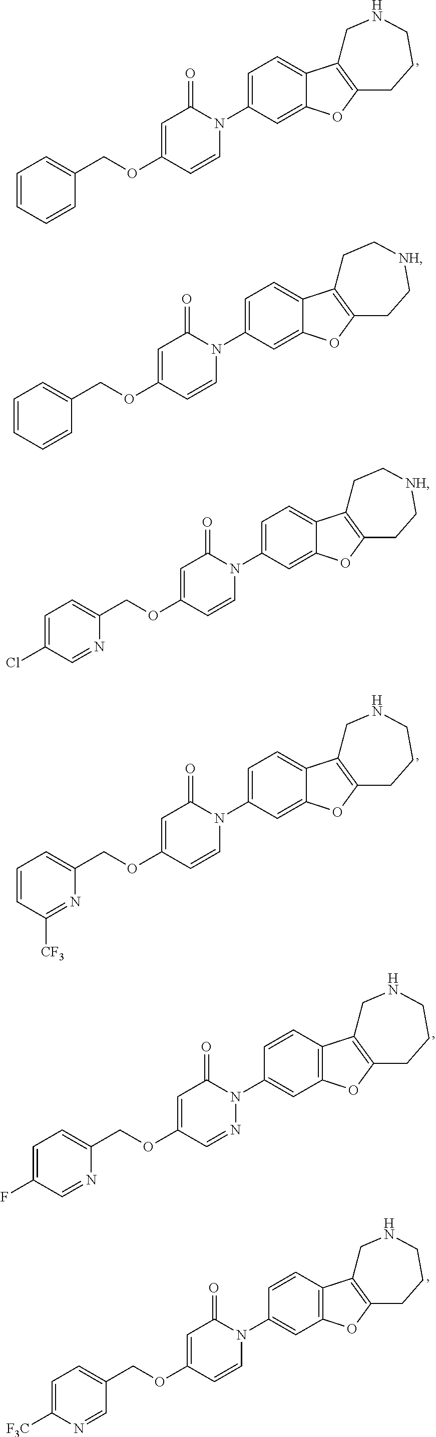 [SMILES: O=c1cc(OCc2ccccc2)ccn1-c1ccc2c3c(oc2c1)CCCNC3 ; O=c1cc(OCc2ccccc2)ccn1-c1ccc2c3c(oc2c1)CCNCC3 ; O=c1cc(OCc2ccc(Cl)cn2)ccn1-c1ccc2c3c(oc2c1)CCNCC3 ; O=c1cc(OCc2cccc(C(F)(F)F)n2)ccn1-c1ccc2c3c(oc2c1)CCCNC3 ; O=c1cc(OCc2ccc(F)cn2)cnn1-c1ccc2c3c(oc2c1)CCCNC3 ; O=c1cc(OCc2ccc(C(F)(F)F)nc2)ccn1-c1ccc2c3c(oc2c1)CCCNC3]

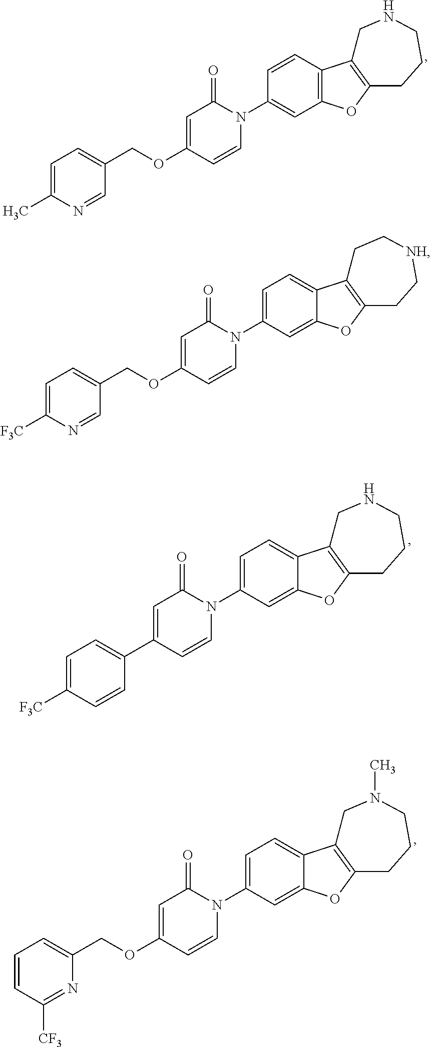 [SMILES: Cc1ccc(COc2ccn(-c3ccc4c5c(oc4c3)CCCNC5)c(=O)c2)cn1 ; O=c1cc(OCc2ccc(C(F)(F)F)nc2)ccn1-c1ccc2c3c(oc2c1)CCNCC3 ; O=c1cc(-c2ccc(C(F)(F)F)cc2)ccn1-c1ccc2c3c(oc2c1)CCCNC3 ; CN1CCCc2oc3cc(-n4ccc(OCc5cccc(C(F)(F)F)n5)cc4=O)ccc3c2C1]

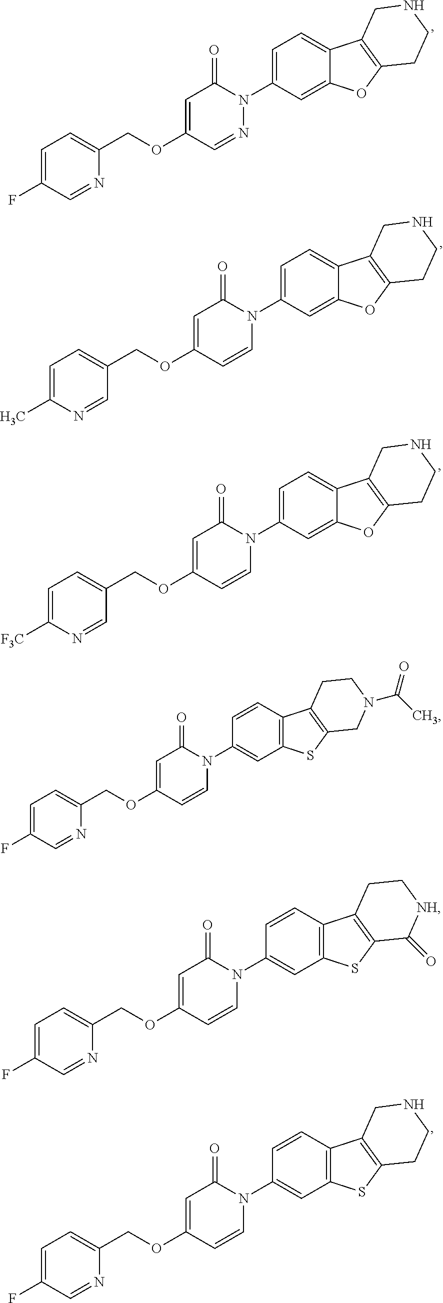 [SMILES: O=c1cc(OCc2ccc(F)cn2)cnn1-c1ccc2c3c(oc2c1)CCNC3 ; Cc1ccc(COc2ccn(-c3ccc4c5c(oc4c3)CCNC5)c(=O)c2)cn1 ; O=c1cc(OCc2ccc(C(F)(F)F)nc2)ccn1-c1ccc2c3c(oc2c1)CCNC3 ; CC(=O)N1CCc2c(sc3cc(-n4ccc(OCc5ccc(F)cn5)cc4=O)ccc23)C1 ; O=C1NCCc2c1sc1cc(-n3ccc(OCc4ccc(F)cn4)cc3=O)ccc21 ; O=c1cc(OCc2ccc(F)cn2)ccn1-c1ccc2c3c(sc2c1)CCNC3]

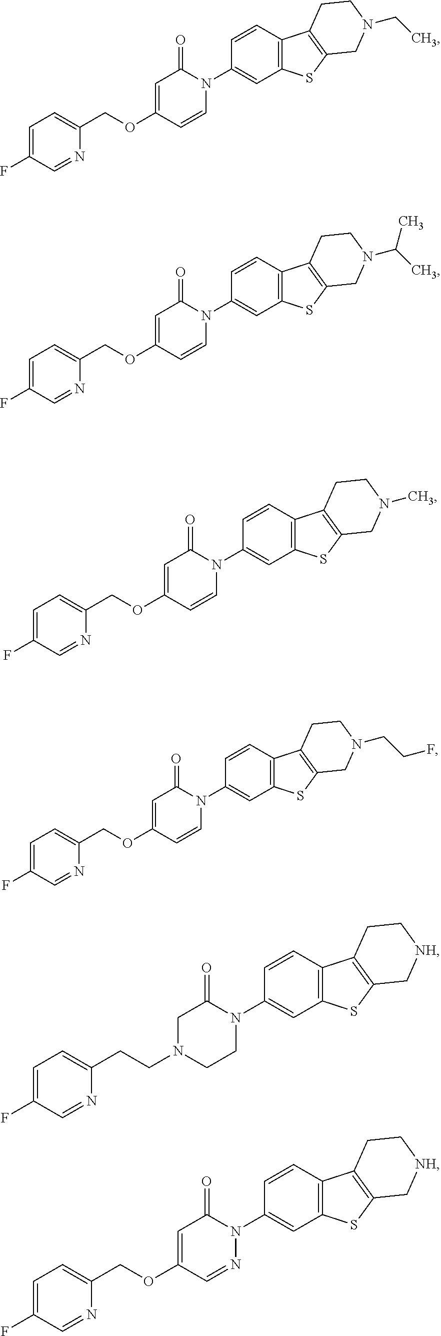 [SMILES: CCN1CCc2c(sc3cc(-n4ccc(OCc5ccc(F)cn5)cc4=O)ccc23)C1 ; CC(C)N1CCc2c(sc3cc(-n4ccc(OCc5ccc(F)cn5)cc4=O)ccc23)C1 ; CN1CCc2c(sc3cc(-n4ccc(OCc5ccc(F)cn5)cc4=O)ccc23)C1 ; O=c1cc(OCc2ccc(F)cn2)ccn1-c1ccc2c3c(sc2c1)CN(CCF)CC3 ; O=C1CN(CCc2ccc(F)cn2)CCN1c1ccc2c3c(sc2c1)CNCC3 ; O=c1cc(OCc2ccc(F)cn2)cnn1-c1ccc2c3c(sc2c1)CNCC3]

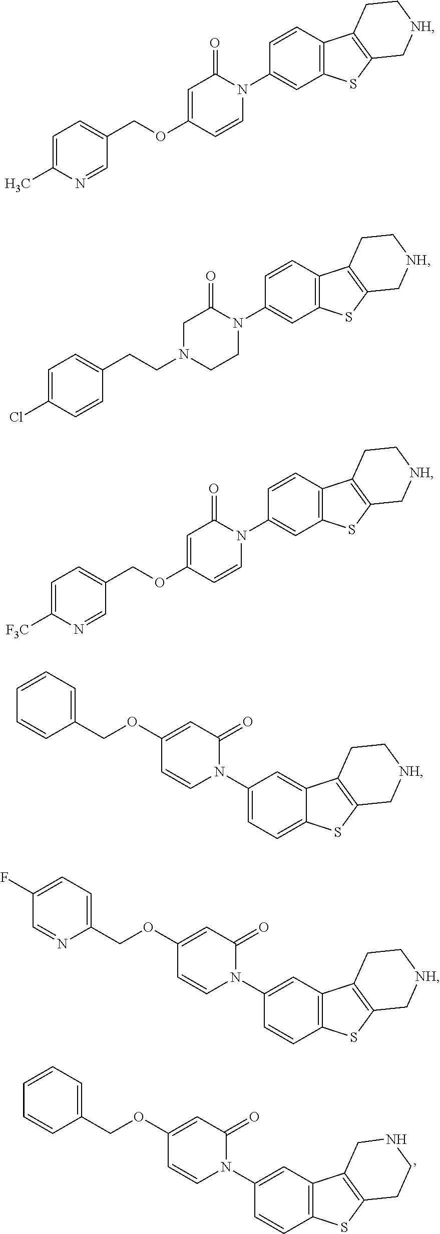 [SMILES: Cc1ccc(COc2ccn(-c3ccc4c5c(sc4c3)CNCC5)c(=O)c2)cn1 ; O=C1CN(CCc2ccc(Cl)cc2)CCN1c1ccc2c3c(sc2c1)CNCC3 ; O=c1cc(OCc2ccc(C(F)(F)F)nc2)ccn1-c1ccc2c3c(sc2c1)CNCC3 ; O=c1cc(OCc2ccccc2)ccn1-c1ccc2sc3c(c2c1)CCNC3 ; O=c1cc(OCc2ccc(F)cn2)ccn1-c1ccc2sc3c(c2c1)CCNC3 ; O=c1cc(OCc2ccccc2)ccn1-c1ccc2sc3c(c2c1)CNCC3]

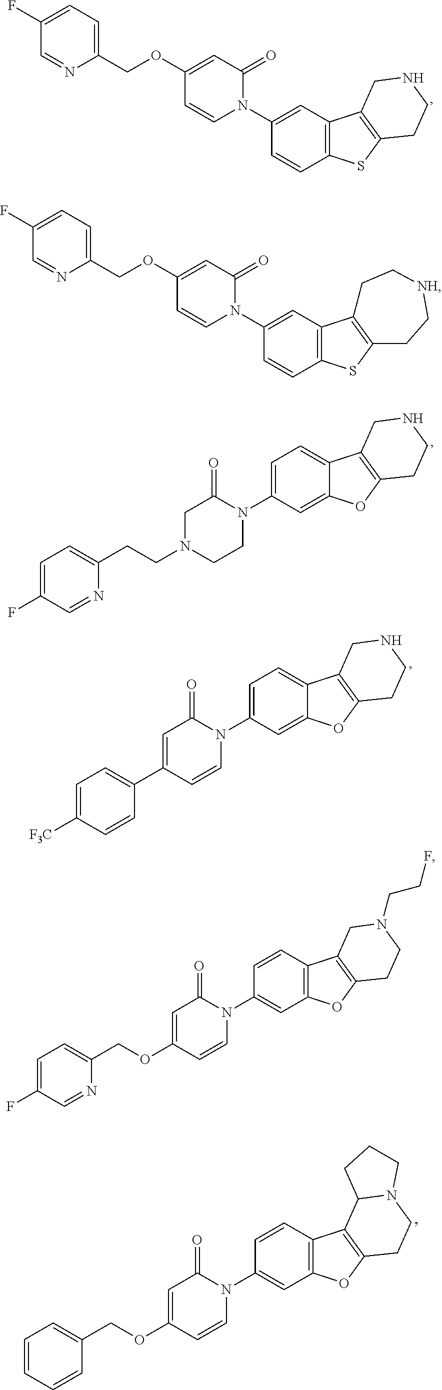 [SMILES: O=c1cc(OCc2ccc(F)cn2)ccn1-c1ccc2sc3c(c2c1)CNCC3 ; O=c1cc(OCc2ccc(F)cn2)ccn1-c1ccc2sc3c(c2c1)CCNCC3 ; O=C1CN(CCc2ccc(F)cn2)CCN1c1ccc2c3c(oc2c1)CCNC3 ; O=c1cc(-c2ccc(C(F)(F)F)cc2)ccn1-c1ccc2c3c(oc2c1)CCNC3 ; O=c1cc(OCc2ccc(F)cn2)ccn1-c1ccc2c3c(oc2c1)CCN(CCF)C3 ; O=c1cc(OCc2ccccc2)ccn1-c1ccc2c3c(oc2c1)CCN1CCCC31]

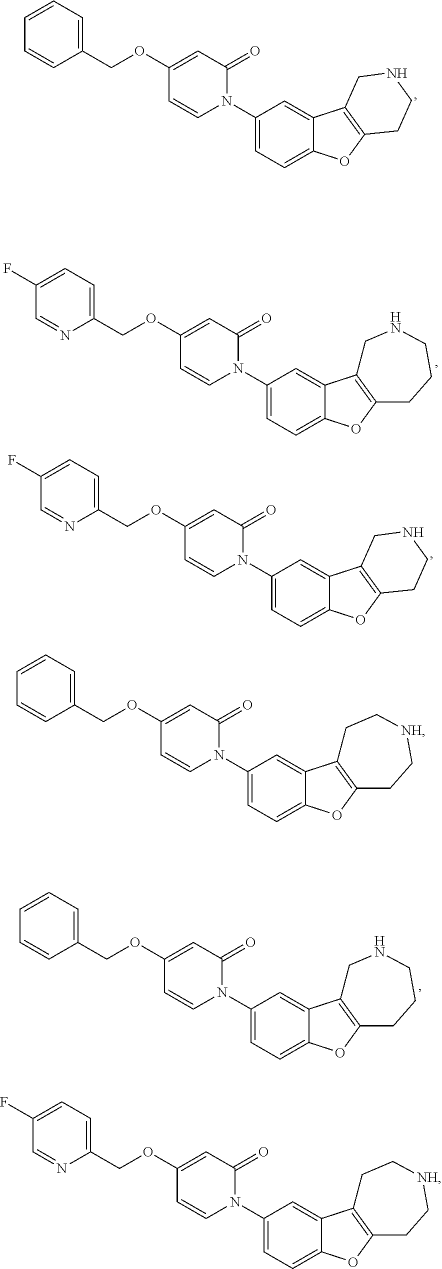 [SMILES: O=c1cc(OCc2ccccc2)ccn1-c1ccc2oc3c(c2c1)CNCC3 ; O=c1cc(OCc2ccc(F)cn2)ccn1-c1ccc2oc3c(c2c1)CNCCC3 ; O=c1cc(OCc2ccc(F)cn2)ccn1-c1ccc2oc3c(c2c1)CNCC3 ; O=c1cc(OCc2ccccc2)ccn1-c1ccc2oc3c(c2c1)CCNCC3 ; O=c1cc(OCc2ccccc2)ccn1-c1ccc2oc3c(c2c1)CNCCC3 ; O=c1cc(OCc2ccc(F)cn2)ccn1-c1ccc2oc3c(c2c1)CCNCC3]

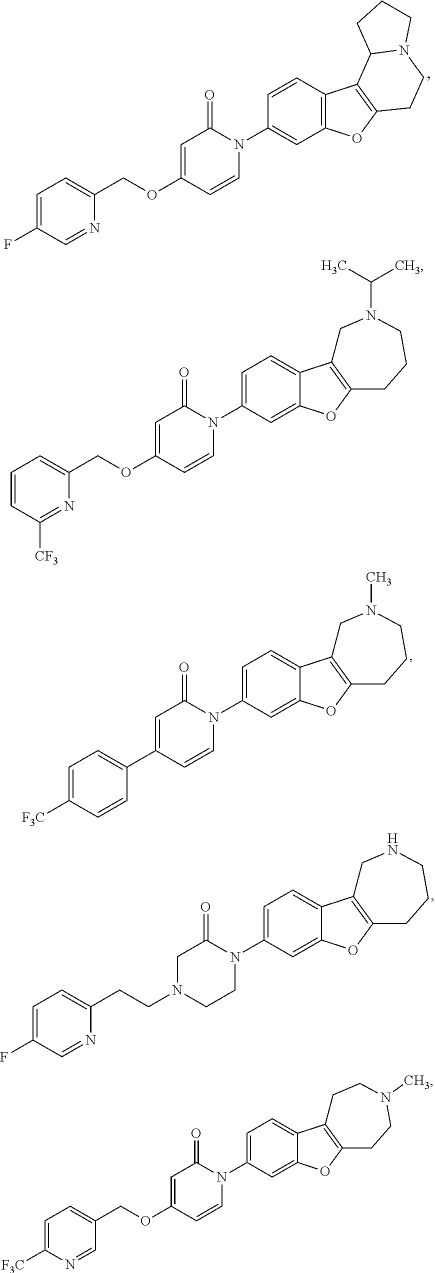 [SMILES: O=c1cc(OCc2ccc(F)cn2)ccn1-c1ccc2c3c(oc2c1)CCN1CCCC31 ; CC(C)N1CCCc2oc3cc(-n4ccc(OCc5cccc(C(F)(F)F)n5)cc4=O)ccc3c2C1 ; CN1CCCc2oc3cc(-n4ccc(-c5ccc(C(F)(F)F)cc5)cc4=O)ccc3c2C1 ; O=C1CN(CCc2ccc(F)cn2)CCN1c1ccc2c3c(oc2c1)CCCNC3 ; CN1CCc2oc3cc(-n4ccc(OCc5ccc(C(F)(F)F)nc5)cc4=O)ccc3c2CC1]

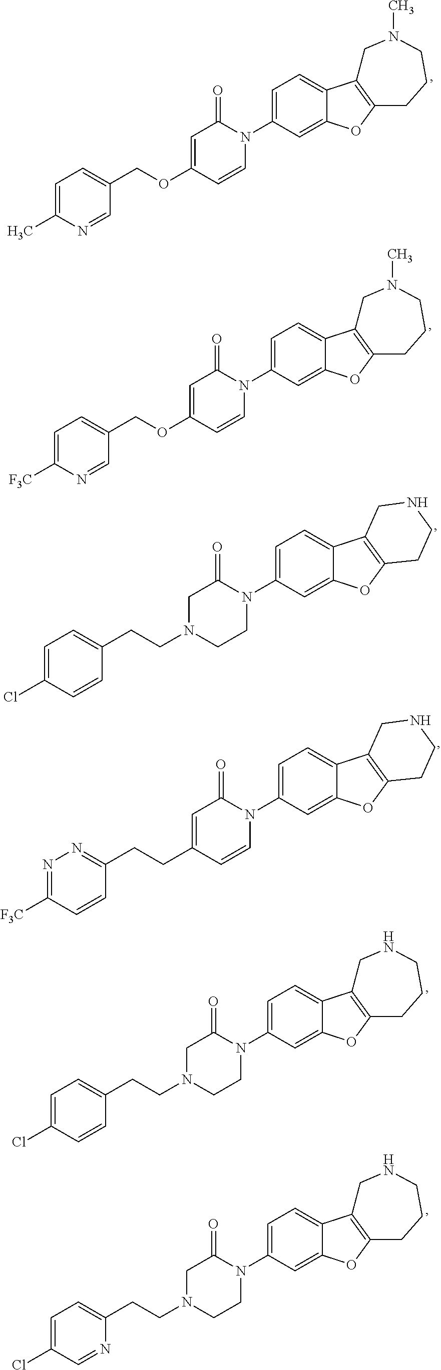 [SMILES: Cc1ccc(COc2ccn(-c3ccc4c5c(oc4c3)CCCN(C)C5)c(=O)c2)cn1 ; CN1CCCc2oc3cc(-n4ccc(OCc5ccc(C(F)(F)F)nc5)cc4=O)ccc3c2C1 ; O=C1CN(CCc2ccc(Cl)cc2)CCN1c1ccc2c3c(oc2c1)CCNC3 ; O=c1cc(CCc2ccc(C(F)(F)F)nn2)ccn1-c1ccc2c3c(oc2c1)CCNC3 ; O=C1CN(CCc2ccc(Cl)cc2)CCN1c1ccc2c3c(oc2c1)CCCNC3 ; O=C1CN(CCc2ccc(Cl)cn2)CCN1c1ccc2c3c(oc2c1)CCCNC3]

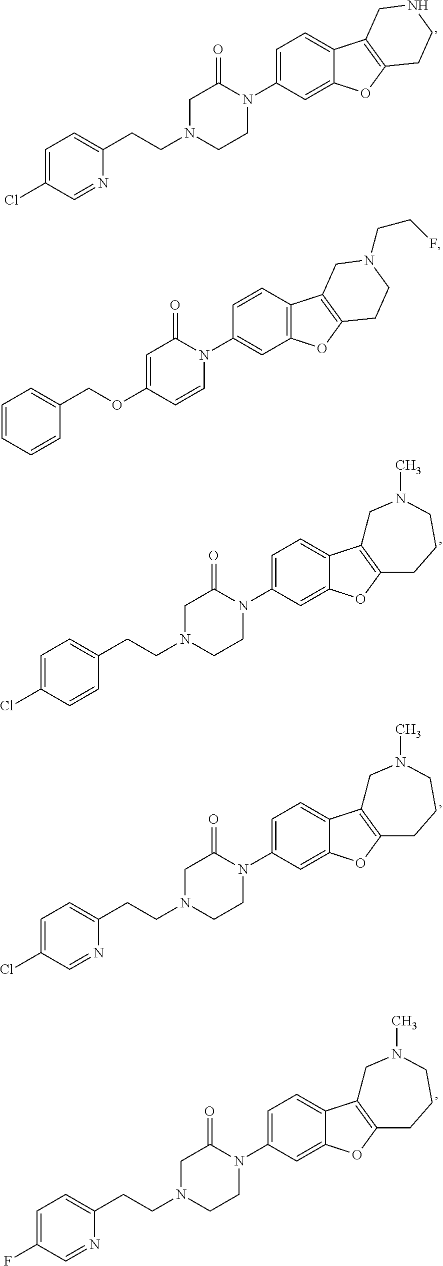 [SMILES: O=C1CN(CCc2ccc(Cl)cn2)CCN1c1ccc2c3c(oc2c1)CCNC3 ; O=c1cc(OCc2ccccc2)ccn1-c1ccc2c3c(oc2c1)CCN(CCF)C3 ; CN1CCCc2oc3cc(N4CCN(CCc5ccc(Cl)cc5)CC4=O)ccc3c2C1 ; CN1CCCc2oc3cc(N4CCN(CCc5ccc(Cl)cn5)CC4=O)ccc3c2C1 ; CN1CCCc2oc3cc(N4CCN(CCc5ccc(F)cn5)CC4=O)ccc3c2C1]

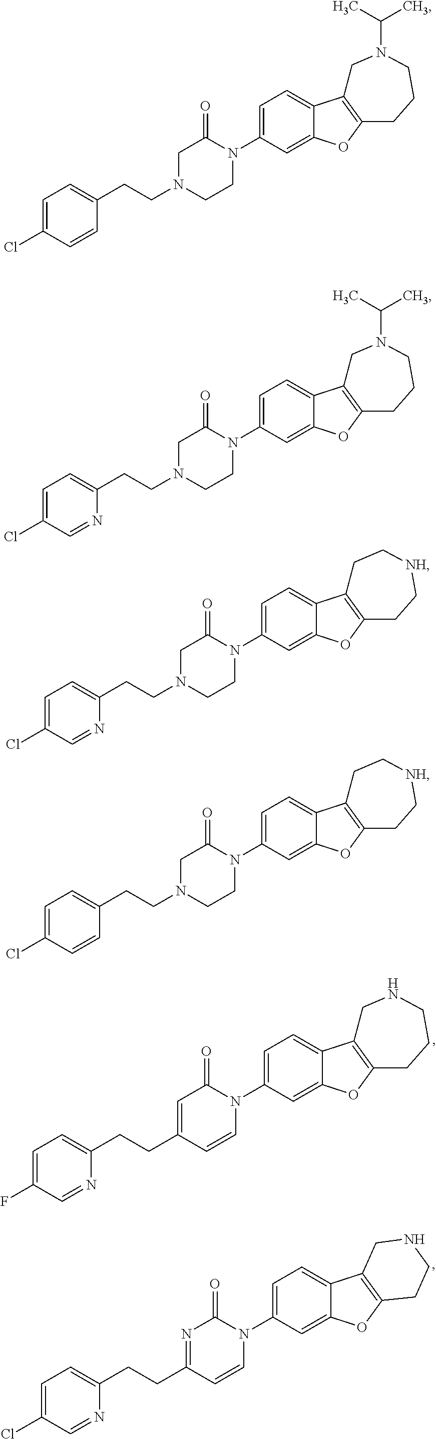 [SMILES: CC(C)N1CCCc2oc3cc(N4CCN(CCc5ccc(Cl)cc5)CC4=O)ccc3c2C1 ; CC(C)N1CCCc2oc3cc(N4CCN(CCc5ccc(Cl)cn5)CC4=O)ccc3c2C1 ; O=C1CN(CCc2ccc(Cl)cn2)CCN1c1ccc2c3c(oc2c1)CCNCC3 ; O=C1CN(CCc2ccc(Cl)cc2)CCN1c1ccc2c3c(oc2c1)CCNCC3 ; O=c1cc(CCc2ccc(F)cn2)ccn1-c1ccc2c3c(oc2c1)CCCNC3 ; O=c1nc(CCc2ccc(Cl)cn2)ccn1-c1ccc2c3c(oc2c1)CCNC3]

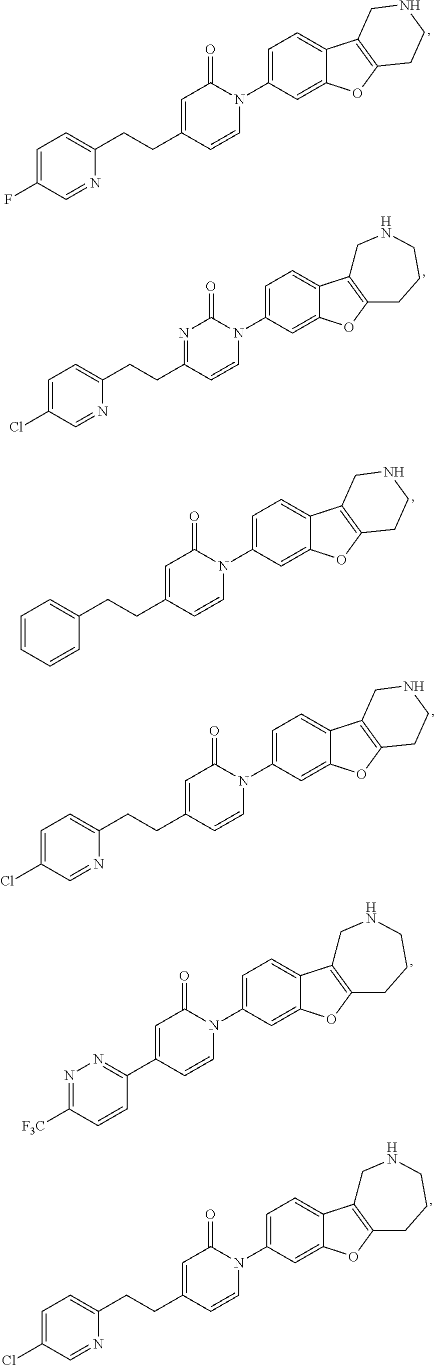 [SMILES: O=c1cc(CCc2ccc(F)cn2)ccn1-c1ccc2c3c(oc2c1)CCNC3 ; O=c1nc(CCc2ccc(Cl)cn2)ccn1-c1ccc2c3c(oc2c1)CCCNC3 ; O=c1cc(CCc2ccccc2)ccn1-c1ccc2c3c(oc2c1)CCNC3 ; O=c1cc(CCc2ccc(Cl)cn2)ccn1-c1ccc2c3c(oc2c1)CCNC3 ; O=c1cc(-c2ccc(C(F)(F)F)nn2)ccn1-c1ccc2c3c(oc2c1)CCCNC3 ; O=c1cc(CCc2ccc(Cl)cn2)ccn1-c1ccc2c3c(oc2c1)CCCNC3]

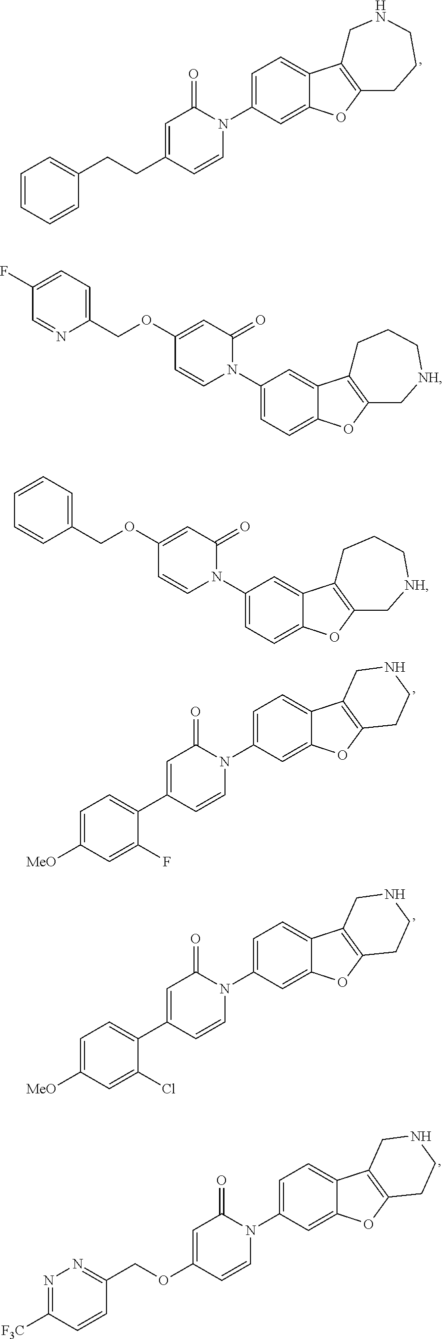

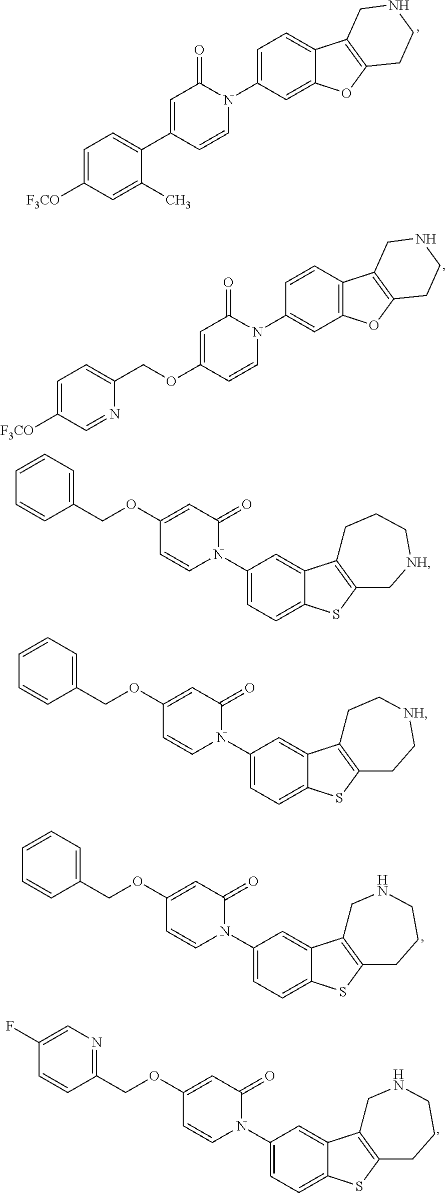

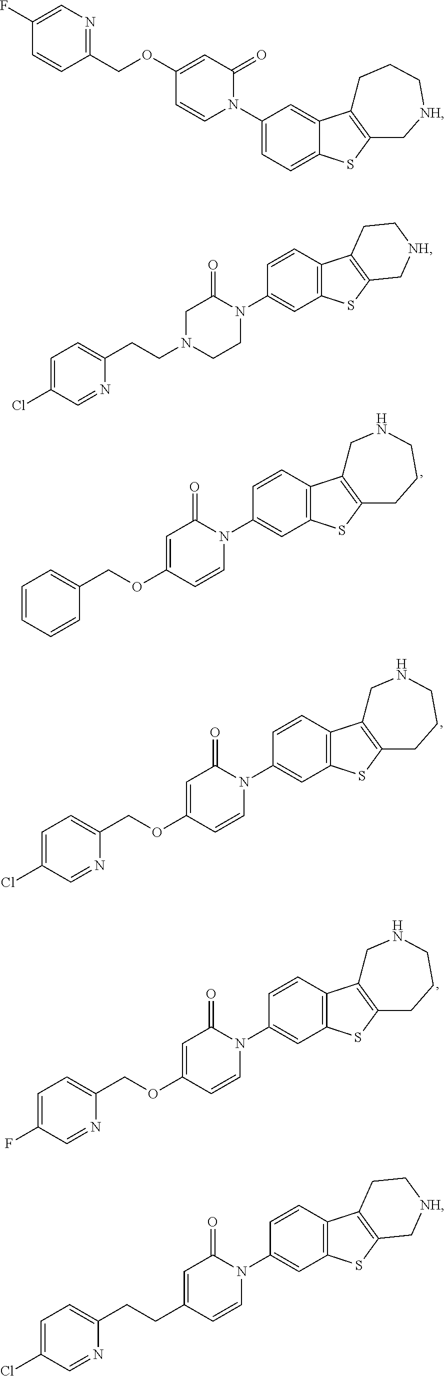 [SMILES: O=c1cc(OCc2ccc(F)cn2)ccn1-c1ccc2sc3c(c2c1)CCCNC3 ; O=C1CN(CCc2ccc(Cl)cn2)CCN1c1ccc2c3c(sc2c1)CNCC3 ; O=c1cc(OCc2ccccc2)ccn1-c1ccc2c3c(sc2c1)CCCNC3 ; O=c1cc(OCc2ccc(Cl)cn2)ccn1-c1ccc2c3c(sc2c1)CCCNC3 ; O=c1cc(OCc2ccc(F)cn2)ccn1-c1ccc2c3c(sc2c1)CCCNC3 ; O=c1cc(CCc2ccc(Cl)cn2)ccn1-c1ccc2c3c(sc2c1)CNCC3]

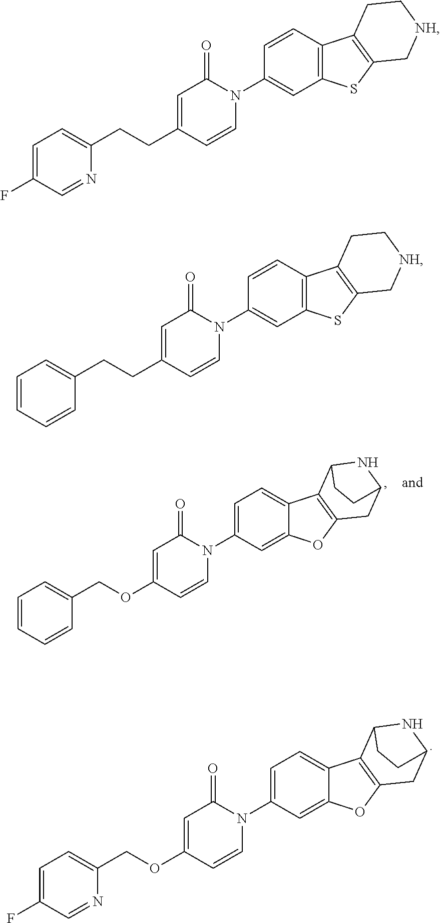 [SMILES: O=c1cc(CCc2ccc(F)cn2)ccn1-c1ccc2c3c(sc2c1)CNCC3 ; O=c1cc(CCc2ccccc2)ccn1-c1ccc2c3c(sc2c1)CNCC3 ; O=c1cc(OCc2ccccc2)ccn1-c1ccc2c3c(oc2c1)CC1CCC3N1 ; O=c1cc(OCc2ccc(F)cn2)ccn1-c1ccc2c3c(oc2c1)CC1CCC3N1]

29. The compound according to claim 1, wherein the compound is selected from the group consisting of

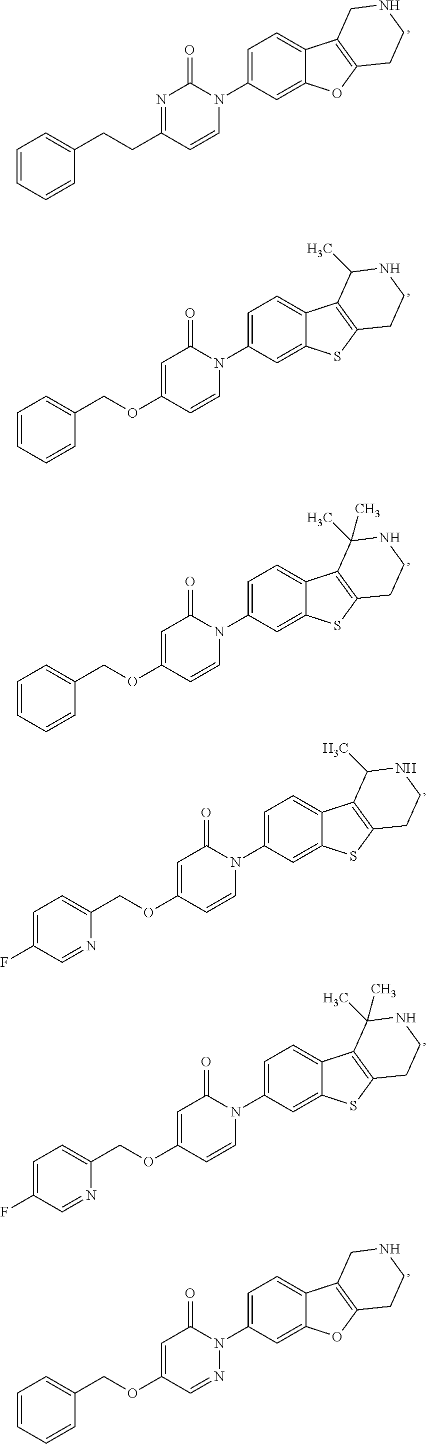 [SMILES: O=c1nc(CCc2ccccc2)ccn1-c1ccc2c3c(oc2c1)CCNC3 ; CC1NCCc2sc3cc(-n4ccc(OCc5ccccc5)cc4=O)ccc3c21 ; CC1(C)NCCc2sc3cc(-n4ccc(OCc5ccccc5)cc4=O)ccc3c21 ; CC1NCCc2sc3cc(-n4ccc(OCc5ccc(F)cn5)cc4=O)ccc3c21 ; CC1(C)NCCc2sc3cc(-n4ccc(OCc5ccc(F)cn5)cc4=O)ccc3c21 ; O=c1cc(OCc2ccccc2)cnn1-c1ccc2c3c(oc2c1)CCNC3]

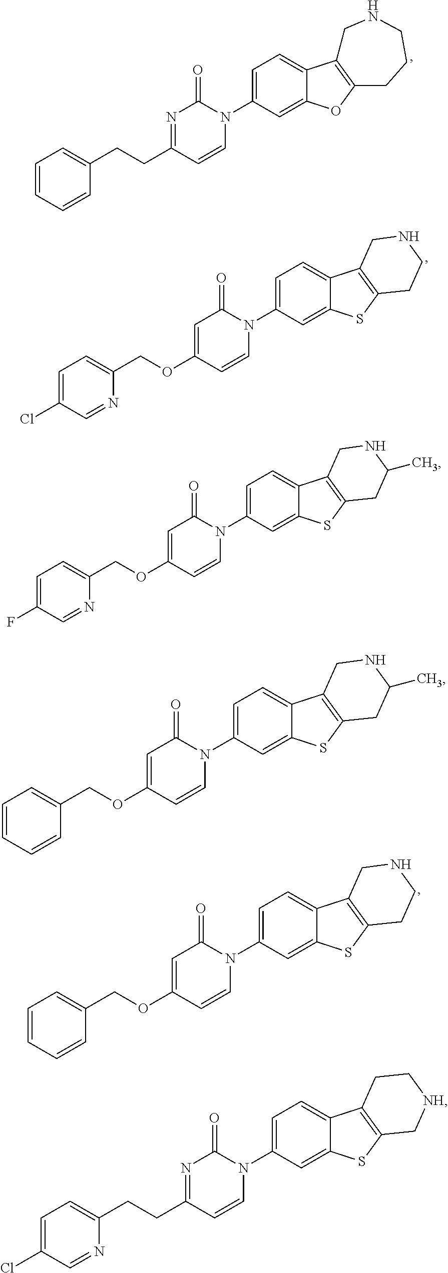 [SMILES: O=c1nc(CCc2ccccc2)ccn1-c1ccc2c3c(oc2c1)CCCNC3 ; O=c1cc(OCc2ccc(Cl)cn2)ccn1-c1ccc2c3c(sc2c1)CCNC3 ; CC1Cc2sc3cc(-n4ccc(OCc5ccc(F)cn5)cc4=O)ccc3c2CN1 ; CC1Cc2sc3cc(-n4ccc(OCc5ccccc5)cc4=O)ccc3c2CN1 ; O=c1cc(OCc2ccccc2)ccn1-c1ccc2c3c(sc2c1)CCNC3 ; O=c1nc(CCc2ccc(Cl)cn2)ccn1-c1ccc2c3c(sc2c1)CNCC3]

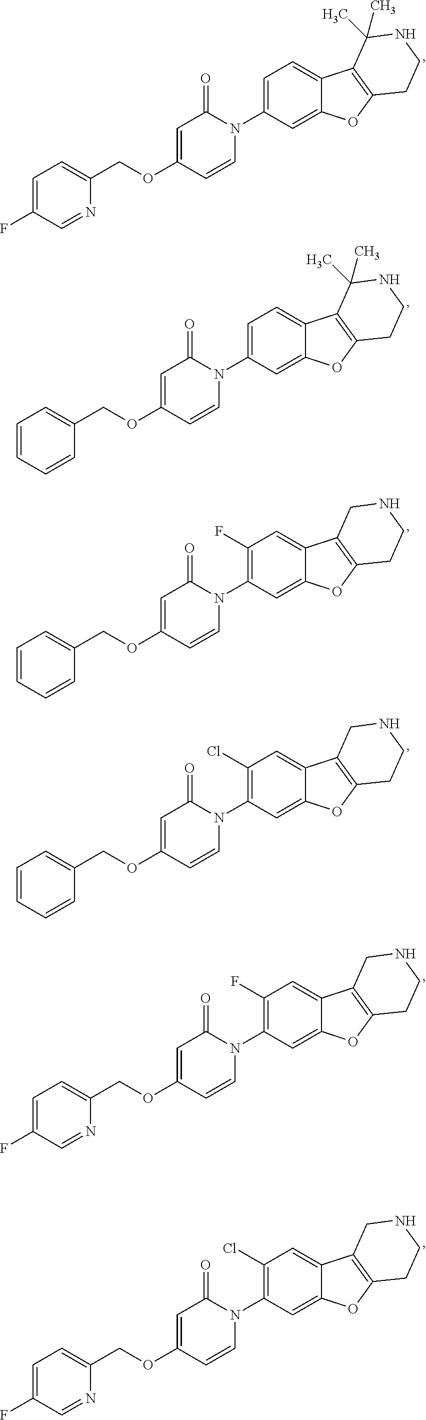 [SMILES: CC1(C)NCCc2oc3cc(-n4ccc(OCc5ccc(F)cn5)cc4=O)ccc3c21 ; CC1(C)NCCc2oc3cc(-n4ccc(OCc5ccccc5)cc4=O)ccc3c21 ; O=c1cc(OCc2ccccc2)ccn1-c1cc2oc3c(c2cc1F)CNCC3 ; O=c1cc(OCc2ccccc2)ccn1-c1cc2oc3c(c2cc1Cl)CNCC3 ; O=c1cc(OCc2ccc(F)cn2)ccn1-c1cc2oc3c(c2cc1F)CNCC3 ; O=c1cc(OCc2ccc(F)cn2)ccn1-c1cc2oc3c(c2cc1Cl)CNCC3]

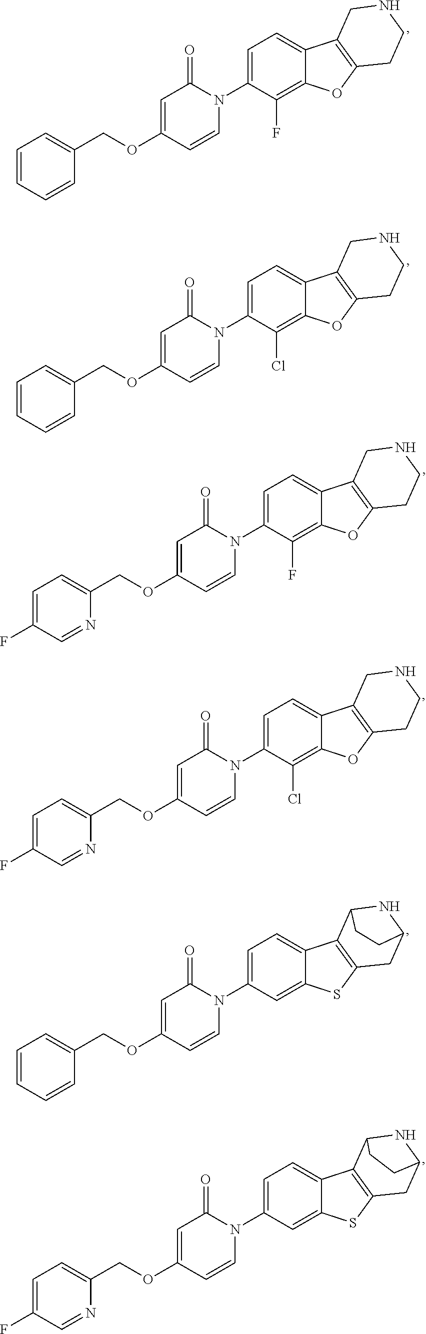 [SMILES: O=c1cc(OCc2ccccc2)ccn1-c1ccc2c3c(oc2c1F)CCNC3 ; O=c1cc(OCc2ccccc2)ccn1-c1ccc2c3c(oc2c1Cl)CCNC3 ; O=c1cc(OCc2ccc(F)cn2)ccn1-c1ccc2c3c(oc2c1F)CCNC3 ; O=c1cc(OCc2ccc(F)cn2)ccn1-c1ccc2c3c(oc2c1Cl)CCNC3 ; O=c1cc(OCc2ccccc2)ccn1-c1ccc2c3c(sc2c1)CC1CCC3N1 ; O=c1cc(OCc2ccc(F)cn2)ccn1-c1ccc2c3c(sc2c1)CC1CCC3N1]

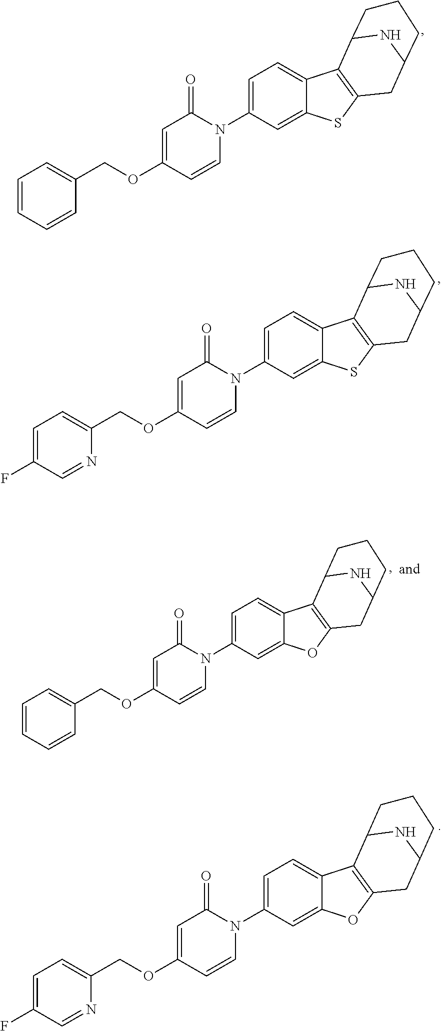 [SMILES: O=c1cc(OCc2ccccc2)ccn1-c1ccc2c3c(sc2c1)CC1CCCC3N1 ; O=c1cc(OCc2ccc(F)cn2)ccn1-c1ccc2c3c(sc2c1)CC1CCCC3N1 ; O=c1cc(OCc2ccccc2)ccn1-c1ccc2c3c(oc2c1)CC1CCCC3N1 ; O=c1cc(OCc2ccc(F)cn2)ccn1-c1ccc2c3c(oc2c1)CC1CCCC3N1]

30. The compound according to claim 1, wherein the compound is an HCl salt.

31. A pharmaceutical composition comprising a therapeutically effective amount of the compound according to claim 1 and a pharmaceutically acceptable carrier.

32. A method of treating a disease or condition which is susceptible to treatment with a MCH-1 receptor antagonist comprising:
selecting a patient with a disease or condition which is susceptible to treatment with a MCH-1 antagonist, and
administering to the patient a therapeutically effective amount of a compound of claim 1 or a pharmaceutically acceptable salt thereof.

33. The method according to claim 32, wherein the disease or condition is selected from the group consisting of obesity, general anxiety disorders, inflammatory bowel disease, social phobias, vertigo, obsessive-compulsive disorders, panic disorders, post-traumatic stress disorders, Parkinson's Disease Psychosis, schizophrenia, cognitive decline and defects in schizophrenia, presenile dementias, Alzheimer's Disease, psychological disorders, depression, substance abuse disorders, dementia associated with neurodegenerative disease, cognition deficits, and epilepsy.

34. The method according to claim 32 further comprising:
administering to the patient a therapeutically effective amount of a therapeutic adjunct.

35. The method according to claim 34, wherein the therapeutic adjunct is selected from the group consisting of phenylpropanolamine, ephedrine, pseudoephedrine, phentermine, a cholecystokinin-A agonist, a monoamine reuptake inhibitor, a sympathomimetic agent, a serotonergic agent, a dopamine agonist, a melanocyte-stimulating hormone receptor agonist or mimetic, a melanocyte-stimulating hormone analog, a cannabinoid receptor antagonist or inverse agonist, a melanin concentrating hormone receptor antagonist, a serotonin 5-HT₆ receptor antagonist, a serotonin 5-HT_{2C} receptor agonist, leptin, a leptin analog, a leptin receptor agonist, amylin peptide, an amylin analog, an amylin receptor agonist, a neuropeptide Y receptor modulator, a galanin antagonist, a GI lipase inhibitor or decreaser, a bombesin agonist, dehydroepiandrosterone or analogs thereof, a glucocorticoid receptor agonist, a glucocorticoid receptor antagonist, an orexin receptor antagonist, an urocortin binding protein antagonist, an agonist of the glucagon-like peptide-1 receptor, a ciliary neurotrophic factor, an allosteric modulator of the GABA_{A} receptor, a serotonin 5-HT_{1A} receptor partial agonist, a selective serotonin reuptake inhibitor, a serotonin-norepinephrine reuptake inhibitor, a monoamine neurotransmitter reuptake inhibitor of tricyclic antidepressant class, a combined serotonin reuptake inhibitor and 5-HT_{2C} antagonist, an H₁ receptor antagonist, a noradrenergic and specific serotonergic antidepressant, a norepinephrine reuptake inhibitor, a norepinephrine-dopamine reuptake inhibitor, a monoamine oxidase inhibitor, an AMP-activated protein kinase agonist, a peroxisome proliferator-activated receptor gamma activator, a HMG-CoA reductase inhibitor, a PDE4 inhibitor, and combinations thereof.

36. A method of treating obesity in a subject in need of weight loss comprising:
selecting a patient in need of weight loss, and
administering to the patient a therapeutically effective amount of a compound of claim 1 or a pharmaceutically acceptable salt thereof.

37. The method according to claim 36 further comprising:
administering to the patient a therapeutically effective amount of an anti-obesity adjunct.

38. The method according to claim 37, wherein the anti-obesity adjunct is selected from the group consisting of phenylpropanolamine, ephedrine, pseudoephedrine, phentermine, a cholecystokinin-A agonist, a monoamine reuptake inhibitor, a sympathomimetic agent, a serotonergic agent, a dopamine agonist, a melanocyte-stimulating hormone receptor agonist or mimetic, a melanocyte-stimulating hormone analog, a cannabinoid receptor antagonist or inverse agonist, a melanin concentrating hormone receptor antagonist, a serotonin 5-HT₆ receptor antagonist, a serotonin 5-HT_{2C} receptor agonist, leptin, a leptin analog, a leptin receptor agonist, amylin peptide, an amylin analog, an amylin receptor agonist, a neuropeptide Y receptor modulator, a galanin antagonist, a GI lipase inhibitor or decreaser, a bombesin agonist, dehydroepiandrosterone or analogs thereof, a glucocorticoid receptor agonist, a glucocorticoid receptor antagonist, an orexin receptor antagonist, an urocortin binding protein antagonist, an agonist of the glucagon-like peptide-1 receptor, a ciliary neurotrophic factor, and combinations thereof.

39. A method of treating obesity in a subject who has experienced weight loss comprising:
selecting a patient who has experienced weight loss, and
administering to the patient a therapeutically effective amount of a compound of claim 1 or a pharmaceutically acceptable salt thereof.

40. A method of treating anxiety comprising:
selecting a patient with anxiety, and
administering to the patient a therapeutically effective amount of a compound of claim 1 or a pharmaceutically acceptable salt thereof.

41. The method according to claim 40 further comprising:
administering to the patient a therapeutically effective amount of a anti-anxiety adjunct.

42. The method according to claim 41, wherein the anti-anxiety adjunct is selected from the group consisting of an allosteric modulator of the GABA_{A} receptor, a serotonin 5-HT_{1A} receptor partial agonist, a selective serotonin reuptake inhibitor, a serotonin-norepinephrine reuptake inhibitor, a monoamine neurotransmitter reuptake inhibitor of tricyclic antidepressant class, a combined serotonin reuptake inhibitor and 5-HT_{2C} antagonist, an H₁ receptor antagonist, and combinations thereof.

43. A method of treating depression comprising:
selecting a patient with depression, and
administering to the patient a therapeutically effective amount of a compound of claim 1 or a pharmaceutically acceptable salt thereof.

44. The method according to claim 43 further comprising:
administering to the patient a therapeutically effective amount of an anti-depression adjunct.

45. The method according to claim 44, wherein the anti-depression adjunct is selected from the group consisting of a serotonin 5-HT_{1A} receptor partial agonist, a selective serotonin reuptake inhibitor, a serotonin-norepinephrine reuptake inhibitor, a monoamine neurotransmitter reuptake inhibitor of tricyclic antidepressant class, a combined serotonin reuptake inhibitor and 5-HT_{2C} antagonist, a noradrenergic and specific serotonergic antidepressant, a norepinephrine reuptake inhibitor, a norepinephrine-dopamine reuptake inhibitor, a monoamine oxidase inhibitor, and combinations thereof.

46. A method of treating non-alcoholic fatty liver disease comprising:
selecting a patient who has non-alcoholic fatty liver disease, and
administering to the patient a therapeutically effective amount of a compound of claim 1 or a pharmaceutically acceptable salt thereof.

47. The method according to claim 46 further comprising:
administering to the patient a therapeutically effective amount of an anti-non-alcoholic fatty liver disease adjunct.

48. The method according to claim 47, wherein the anti-non-alcoholic fatty liver disease adjunct is selected from the group consisting of an AMP-activated protein kinase agonist, a peroxisome proliferator-activated receptor gamma activator, a HMG-CoA reductase inhibitor, a PDE4 inhibitor, and combinations thereof.

49. A method of treating inflammatory bowel disease comprising:
selecting a patient with inflammatory bowel disease, and
administering to the patient a therapeutically effective amount of a compound of claim 1 or a pharmaceutically acceptable salt thereof.

50. A process for the preparation of a product compound of formula (I): 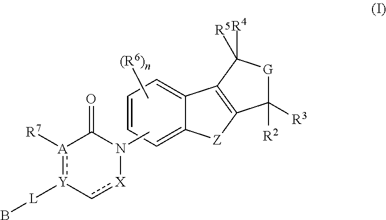
wherein
R²-R⁵ and R⁹-R¹² are each, independently, selected from the group consisting of H, halogen, —OR¹³, —NR¹³R¹⁴, —NR¹³C(O)R¹⁴, —NR¹³C(O)₂R¹⁴, —NR¹⁵C(O)NR¹⁴R¹⁵, —S(O)_{q}R¹⁴, —CN, —C(O)R¹⁴, —C(O)NR¹³R¹⁴, C₁-C₆ alkyl, C₂-C₆ alkenyl, C₂-C₆ alkynyl, C₃-C₆ cycloalkyl, C₄-C₇ cycloalkylalkyl, heterocyclyl, aryl, and heteroaryl, wherein each of C₁-C₆ alkyl, C₂-C₆ alkenyl, C₂-C₆ alkynyl, C₃-C₆ cycloalkyl, C₄-C₇ cycloalkylalkyl, heterocyclyl, aryl, and heteroaryl is optionally substituted with from 1 to 3 substituents independently selected at each occurrence thereof from C₁-C₃ alkyl, halogen, —CN, —OR¹⁶, —NR¹⁶R¹⁷, and phenyl which is optionally substituted 1-3 times with halogen, C₁-C₄ alkyl, C₁-C₄ haloalkyl, C₁-C₄ alkoxy, —CN, —OR¹⁶, or —NR¹⁶R¹⁷; or R² and R³ or R⁴ and R⁵ can combine to form an oxo, thio, imine, cycloalkyl, or heterocycle group containing from 1 to 5 heteroatoms selected from the group consisting of oxygen, nitrogen, and sulfur; or any one of R², R³, R⁴, or R⁵ can combine with any one of R⁹, R¹⁰, R¹¹, or R¹² to form —(CH₂)ᵣ—;
R⁶ is independently selected at each location from the group consisting of H, halogen, —OR¹³, —NR¹³C(O)R¹⁴, —NR¹³C(O)₂R¹⁴, —NR¹⁵C(O)NR¹⁴R¹⁵, —S(O)_{q}R¹⁴, —CN, —C(O)R¹⁴, —C(O)NR¹³R¹⁴, C₁-C₆ alkyl, C₂-C₆ alkenyl, C₂-C₆ alkynyl, C₃-C₆ cycloalkyl, C₄-C₇ cycloalkylalkyl, heterocyclyl, aryl, and heteroaryl, wherein each of C₁-C₆ alkyl, C₂-C₆ alkenyl, C₂-C₆ alkynyl, C₃-C₆ cycloalkyl, C₄-C₇ cycloalkylalkyl, heterocyclyl, aryl, and heteroaryl is optionally substituted with from 1 to 3 substituents independently selected at each occurrence thereof from C₁-C₃ alkyl, halogen, —CN, —OR⁶, —NR¹⁶R¹⁷, and phenyl which is optionally substituted 1-3 times with halogen, C₁-C₄ alkyl, C₁-C₄ haloalkyl, C₁-C₄ alkoxy, —CN, —OR¹⁶, or NR¹⁶R¹⁷;
R⁷ is optionally present and, if present, is selected from the group consisting of H, halogen, —OR¹³, —NR¹³R¹⁴, —NR¹³C(O)R¹⁴, —NR¹³C(O)₂R¹⁴, —NR¹⁵C(O)NR¹⁴R¹⁵, —S(O)_{q}R¹⁴, —CN, —C(O)R¹⁴, —C(O)NR¹³R¹⁴, C₁-C₆ alkyl, C₂-C₆ alkenyl, C₂-C₆ alkynyl, C₃-C₆ cycloalkyl, C₄-C₇ cycloalkylalkyl, heterocyclyl, aryl, and heteroaryl, wherein each of C₁-C₆ alkyl, C₂-C₆ alkenyl, C₂-C₆ alkynyl, C₃-C₆ cycloalkyl, C₄-C₇ cycloalkylalkyl, heterocyclyl, aryl, and heteroaryl is optionally substituted with from 1 to 3 substituents independently selected at each occurrence thereof from C₁-C₃ alkyl, halogen, —CN, —OR¹⁶, —NR¹⁶R¹⁷, and phenyl which is optionally substituted 1-3 times with halogen, C₁-C₄ alkyl, C₁-C₄ haloalkyl, C₁-C₄ alkoxy, —CN, —OR¹⁶, or —NR¹⁶R¹⁷;
R⁸ is selected from the group consisting of H, —S(O)_{q}R¹⁴, —C(O)R¹⁴, —C(O)NR¹³R¹⁴, C₁-C₆ alkyl, C₂-C₆ alkenyl, C₂-C₆ alkynyl, C₃-C₆ cycloalkyl, C₄-C₇ cycloalkylalkyl, heterocyclyl, and heteroaryl, wherein each of C₁-C₆ alkyl, C₂-C₆ alkenyl, C₂-C₆ alkynyl, C₃-C₆ cycloalkyl, C₄-C₇ cycloalkylalkyl, heterocyclyl, and heteroaryl is optionally substituted with from 1 to 3 substituents independently selected at each occurrence thereof from C₁-C₃ alkyl, halogen, —CN, —OR¹⁶, —NR¹⁶R¹⁷, and phenyl which is optionally substituted 1-3 times with halogen, C₁-C₄ alkyl, C₁-C₄ haloalkyl, C₁-C₄ alkoxy, —CN, —OR¹⁶, or NR¹⁶R¹⁷; or
R⁸ and one of R², R³, R⁴, and R⁵ can combine to form a 3- to 7-membered heterocycle, wherein the 3- to 7-membered heterocycle includes from 1 to 2 heteroatoms selected from the group consisting of N, O, and S and is optionally substituted with from 1 to 10 substituents independently selected at each occurrence thereof from H, halogen, —OR¹³, —NR¹³R¹⁴, —NR¹³C(O)R¹⁴, —NR¹³C(O)₂R¹⁴, —NR¹⁵C(O)NR¹⁴R¹⁵, —S(O)_{q}R¹⁴, —CN, —C(O)R¹⁴, —C(O)NR¹³R¹⁴, C₁-C₆ alkyl, C₂-C₆ alkenyl, C₂-C₆ alkynyl, C₃-C₆ cycloalkyl, C₄-C₇ cycloalkylalkyl, heterocyclyl, aryl, and heteroaryl, wherein each of C₁-C₆ alkyl, C₂-C₆ alkenyl, C₂-C₆ alkynyl, C₃-C₆ cycloalkyl, C₄-C₇ cycloalkylalkyl, heterocyclyl, aryl, and heteroaryl is optionally substituted with from 1 to 3 substituents independently selected at each occurrence thereof from C₁-C₃ alkyl, halogen, —CN, —OR¹⁶, —NR¹⁶R¹⁷, and phenyl which is optionally substituted 1-3 times with halogen, C₁-C₄ alkyl, C₁-C₄ haloalkyl, C₁-C₄ alkoxy, —CN, —OR¹⁶, or —NR¹⁶R¹⁷;
R¹³ is H, C₁-C₄ alkyl, C₁-C₄ haloalkyl, C₁-C₄ alkoxyalkyl, C₃-C₆ cycloalkyl, C₄-C₇ cycloalkylalkyl, —C(O)R¹⁵, phenyl, or benzyl, wherein phenyl or benzyl is optionally substituted 1 to 3 times with halogen, cyano, C₁-C₄ alkyl, C₁-C₄ haloalkyl, or C₁-C₄ alkoxy;
R¹⁴ is H, C₁-C₄ alkyl, C₁-C₄ haloalkyl, C₁-C₄ alkoxyalkyl, C₃-C₆ cycloalkyl, C₄-C₇ cycloalkylalkyl, phenyl, or benzyl, wherein phenyl or benzyl is optionally substituted 1 to 3 times with halogen, cyano, C₁-C₄ alkyl, C₁-C₄ haloalkyl, or C₁-C₄ alkoxy;
R¹⁵ is C₁-C₄ alkyl, C₁-C₄ haloalkyl, or phenyl;
R¹⁶ and R¹⁷ are each independently H, C₁-C₄ alkyl, C₁-C₄ haloalkyl, C₁-C₄ alkoxyalkyl, C₃-C₆ cycloalkyl, C₄-C₇ cycloalkylalkyl, —C(O)R¹⁵, phenyl, or benzyl, wherein phenyl or benzyl is optionally substituted from 1 to 3 times with a substituent selected independently at each occurrence thereof from the group consisting of halogen, cyano, C₁-C₄ alkyl, C₁-C₄ haloalkyl, and C₁-C₄ alkoxy;
R¹⁸ is selected from the group consisting of H, halogen, —OR¹³, —NR¹³R¹⁴—NR¹³C(O)R¹⁴, —NR¹³C(O)₂R¹⁴, —NR¹⁴C(O)NR¹⁴R¹⁵, —S(O)_{q}R¹⁴, —CN, —C(O)R¹⁴, —C(O)NR¹³R¹⁴, C₁-C₆ alkyl, C₂-C₆ alkenyl, C₂-C₆ alkynyl, C₃-C₆ cycloalkyl, C₄-C₇ cycloalkylalkyl, heterocyclyl, aryl, and heteroaryl, wherein each of C₁-C₆ alkyl, C₂-C₆ alkenyl, C₂-C₆ alkynyl, C₃-C₆ cycloalkyl, C₄-C₇ cycloalkylalkyl, heterocyclyl, aryl, and heteroaryl is optionally substituted with from 1 to 3 substituents independently selected at each occurrence thereof from C₁-C₃ alkyl, halogen, —CN, —OR¹⁶, —NR¹⁶R¹⁷, and phenyl which is optionally substituted 1-3 times with halogen, C₁-C₄ alkyl, C₁-C₄ haloalkyl, C₁-C₄ alkoxy, —CN, —OR⁸, or —NR⁸R⁹;
G is —NR⁸—CR⁹R¹⁰—, —CR⁹R¹⁰—NR⁸—, —NR⁸—CR⁹R¹⁰—CR¹¹R¹²—, —CR⁹R¹⁰—NR⁸—CR¹¹R¹²—, or —CR⁹R¹⁰—CR¹¹R¹²—NR⁸—;
X is CR¹⁸, C(R¹⁸)₂, N, or NR¹⁸;
Y is CR¹⁸, C, or N;
Z is O or S;
L is —(CH₂)ₚ—O—, —(CH₂)ₚ—, —CH═CH—, or a bond;
A is C, CH, or N;
B is aryl, heteroaryl, heterocyclyl, or cycloalkyl, wherein each of the aryl, heteroaryl, heterocyclyl, or cycloalkyl is optionally substituted with from 1 to 3 substituents selected from the group consisting of H, alkoxy, —S-alkyl, optionally substituted C₁-C₆ alkyl, halogen, —CF₃, —OCF₃, and —CN;
n is 0, 1, 2, or 3;
p is from 1 to 4;
q is 0, 1, or 2;
r is from 1 to 4; and [symbol]
represents an optional double bond,
said process comprising:
treating a first intermediate of formula (II): 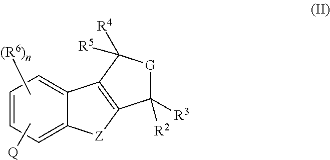
wherein Q is a halogen, under conditions effective to form the compound of formula (I).

51. The process according to claim 50, wherein treating comprises:
reacting the first intermediate with a second intermediate of formula (III):

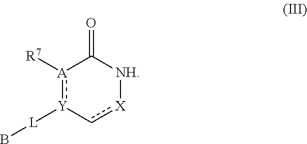

52. The process according to claim 50 further comprising:
treating a third intermediate of formula (IV): 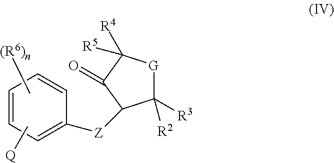
under conditions effective to form the first intermediate compound.

53. The process according to claim 52 further comprising:
reacting 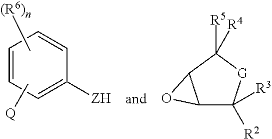
under conditions effective to form a fourth intermediate of formula (V): 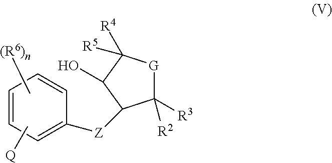
and
treating the fourth intermediate compound under conditions effective to form the third intermediate compound.
